(19) United States
(12) Patent Application Publication
(10) Pub. No.: US 2024/0298527 A1
(43) Pub. Date: Sep. 5, 2024

(54) HETEROCYCLIC COMPOUND AND ORGANIC LIGHT EMITTING DEVICE COMPRISING SAME

(71) Applicant: LT MATERIALS CO., LTD., Yongin-si, Gyeonggi-do (KR)

(72) Inventors: Nam-Jin LEE, Yongin-si (KR); Na-Yeong KIM, Yongin-si (KR); Won-Jang JEONG, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/289,950

(22) PCT Filed: Feb. 17, 2022
(86) PCT No.: PCT/KR2022/002321

§ 371 Date: Nov. 8, 2023

(30) Foreign Application Priority Data
Jun. 7, 2021 (KR) 10-2021-0073284

(51) Int. Cl. H10K 85/60; C07C 211/61; C07D 307/91; C07D 333/76; C09K 11/06; H10K 50/11; H10K 50/15; H10K 50/17; H10K 50/18

(52) U.S. Cl. CPC H10K 85/633; C07C 211/61; C07D 307/91; C07D 333/76; C09K 11/06; H10K 85/626; H10K 85/636; C07C 2603/18; C07C 2603/26; C07C 2603/42; C07C 2603/97; C09K 2211/1003; H10K 50/11; H10K 50/15; H10K 50/171; H10K 50/181; H10K 85/622; H10K 85/624; H10K 85/6574; H10K 85/6576

(57) ABSTRACT

The present specification provides a heterocyclic compound represented by Chemical Formula 1 and an organic light emitting device comprising the same.

### TECHNICAL FIELD

The present specification relates to a heterocyclic compound and an organic light emitting device comprising the same.

This application claims priority to and the benefit of Korean Patent Application No. 10-2021-0073284 filed in the Korean Intellectual Property Office on Jun. 7, 2021, the entire contents of which are incorporated herein by reference.

### BACKGROUND ART

An organic electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

It is necessary to perform studies on an organic light emitting device comprising a compound having a chemical structure, which may satisfy conditions required for a material which is available for the organic light emitting device, for example, appropriate energy levels, electrochemical stability, thermal stability, and the like, and may perform various functions required for the organic light emitting device according to the substituent.

### DISCLOSURE

### Technical Problem

The present application relates to a heterocyclic compound and an organic light emitting device comprising the same.

### Technical Solution

In an exemplary embodiment of the present application, provided is a heterocyclic compound represented by the following Chemical Formula 1.

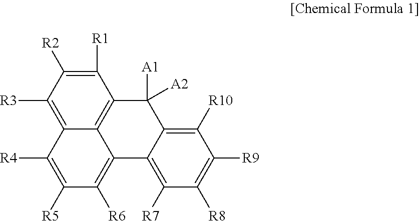

In Chemical Formula 1,
A1 and A2 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
R1 to R5 and R7 to R10 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; —P(═O)RR′; the following Chemical Formula 1-1 and the following Chemical Formula 1-2,
R6 is represented by the following Chemical Formula 1-1; or the following Chemical Formula 1-2,

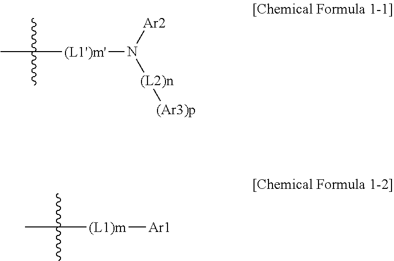

In Chemical Formulae 1-1 and 1-2,
L1, L1′ and L2 are the same as or different from each other, and are each independently a direct bond; a substituted or unsubstituted C6 to C60 arylene group; or a substituted or unsubstituted C2 to C60 heteroarylene group,
Ar1 to Ar3 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
m, m′ and n are each an integer from 0 to 3,
p is an integer from 1 to 5,
when m, m′, n and p are each 2 or higher, substituents in the parenthesis are the same as or different from each other, and
R, R′ and R″ are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

Further, according to an exemplary embodiment of the present application, provided is an organic light emitting device comprising: a first electrode; a second electrode provided to face the first electrode; and an organic material layer having one or more layers provided between the first electrode and the second electrode, in which one or more layers of the organic material layer comprise one or more of the heterocyclic compound represented by Chemical Formula 1.

### Advantageous Effects

The compound described in the present specification can be used as a material for the organic material layer of the organic light emitting device. The compound can serve as a hole injection material, a hole transport material, a light emitting material, an electron transport material, an electron injection material, an electron blocking material, a hole blocking material, and the like in an organic light emitting device. In particular, the compound can be used as a hole transport material, electron blocking material or light emitting material for an organic light emitting device.

### DESCRIPTION OF DRAWINGS

FIGS. 1 to 4 each are views schematically illustrating a stacking structure of an organic light emitting device according to an exemplary embodiment of the present application.

FIG. 5 is a view illustrating a description on the exciplex phenomenon.

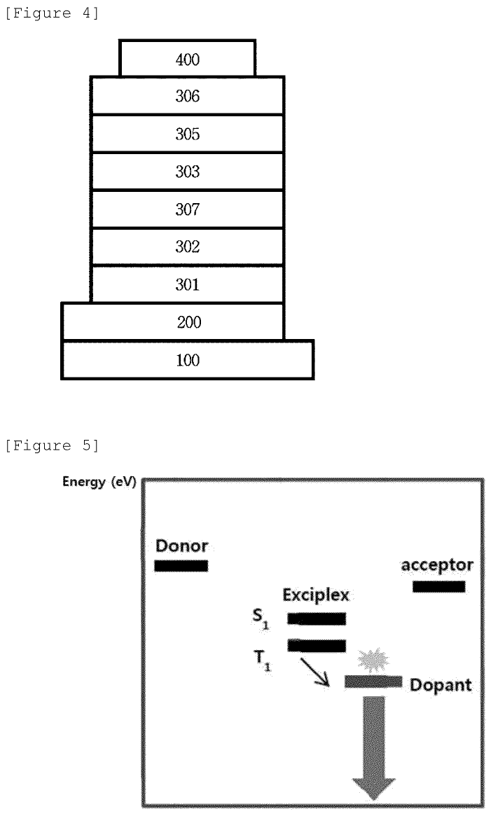

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
307: Electron blocking layer
400: Negative electrode

### MODE FOR INVENTION

Hereinafter, the present application will be described in detail.

In the present invention, “when a substituent is not indicated in the structure of a chemical formula or compound” means that a hydrogen atom is bonded to a carbon atom. However, since deuterium (²H) and tritium (³H) are isotopes of hydrogen, some hydrogen atoms may be deuterium or tritium.

In an exemplary embodiment of the present invention, “when a substituent is not indicated in the structure of a chemical formula or compound” may mean that all the positions that may be reached by the substituent are hydrogen, deuterium or tritium. That is, deuterium and tritium are isotopes of hydrogen, some hydrogen atoms may be deuterium and tritium which are isotopes thereof, and in this case, the content of deuterium or tritium may be 0% to 100%.

In an exemplary embodiment of the present invention, in “the case where a substituent is not indicated in the structure of a chemical formula or compound”, when the substituents do not explicitly exclude deuterium and tritium, such as “the content of deuterium is 0%”, “the content of tritium is 0%”, “the content of hydrogen is 100%”, and “the substituents are all hydrogen”, hydrogen, deuterium and tritium may be mixed and used in the compound.

In an exemplary embodiment of the present invention, deuterium is one of the isotopes of hydrogen, is an element that has a deuteron composed of one proton and one neutron as a nucleus, and may be represented by hydrogen-2, and the element symbol may also be expressed as D or ²H. Similarly, the element symbol for tritium may also be expressed as T or ³H.

In an exemplary embodiment of the present invention, the isotope means an atom with the same atomic number (Z), but different mass numbers (A), and the isotope may be interpreted as an element which has the same number of protons, but different number of neutrons.

In an exemplary embodiment of the present invention, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

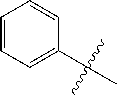 [SMILES: *c1ccccc1]

may mean the case where the total number of substituents that the phenyl group can have is 5 (T1 in the formula) and the number of deuteriums among the substituents is 1 (T2 in the formula). That is, a deuterium content of 20% in the phenyl group may be represented by the following structural formula.

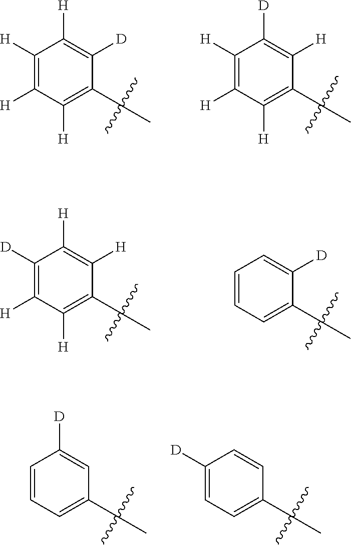 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not comprise a deuterium atom, that is, has five hydrogen atoms.

In the present specification, the halogen may be fluorine, chlorine, bromine or iodine.

In the present specification, an alkyl group comprises a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group comprises a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, an alkynyl group comprises a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof comprise methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, a cycloalkyl group comprises a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group comprises O, S, Se, N, or Si as a heteroatom, comprises a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group comprises a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group comprises a spiro group. The number of carbon atoms of the aryl group may be 6 to 60, specifically 6 to 40, and more specifically 6 to 25. Specific examples of the aryl group comprise a phenyl group, a biphenyl group, a terphenyl group, a naphthyl group, an anthracenyl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the fluorenyl group may be substituted, and adjacent substituents may be bonded to each other to form a ring.

When the fluorenyl group is substituted, the substituent may be the following structure, but is not limited thereto.

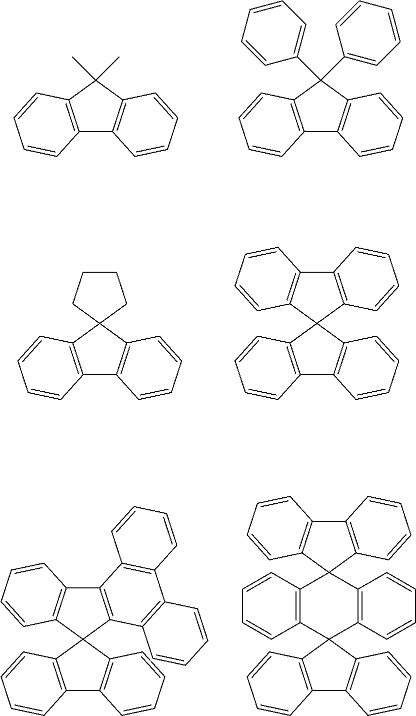

In the present specification, a heteroaryl group comprises S, O, Se, N, or Si as a heteroatom, comprises a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole) group, a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydrodibenzo[b, f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, an amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, an arylene group means that there are two bonding positions in an aryl group, that is, a divalent group. The above-described description on the aryl group may be applied to the arylene group, except that the arylene groups are each a divalent group. Further, a heteroarylene group means that there are two bonding positions in a heteroaryl group, that is, a divalent group. The above-described description on the heteroaryl group may be applied to the heteroarylene group, except for a divalent heteroarylene group.

In the present specification, a phosphine oxide group is represented by —P(═O) R101R102, and R101 and R102 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the phosphine oxide group comprise a diphenylphosphine oxide group, dinaphthylphosphine oxide group, and the like, but are not limited thereto.

In the present specification, a silyl group comprises Si and is a substituent to which the Si atom is directly linked as a radical, and is represented by —SiR104R105R106, and R104 to R106 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the silyl group comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

Structures exemplified by the above-describe cycloalkyl group, aryl group, cycloheteroalkyl group and heteroaryl group may be applied, except that an aliphatic or aromatic hydrocarbon ring, or an aliphatic or aromatic hetero ring which adjacent groups may form is not a monovalent group.

In the present specification, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more substituents are substituted, the two or more substituents may be the same as or different from each other.

In the present specification, “substituted or unsubstituted” means being substituted with one or more substituents selected from the group consisting of deuterium; a halogen group; a cyano group; a C1 to C60 straight-chained or branched alkyl; a C2 to C60 straight-chained or branched alkenyl; a C2 to C60 straight-chained or branched alkynyl; a C3 to C60 monocyclic or polycyclic cycloalkyl; a C2 to C60 monocyclic or polycyclic heterocycloalkyl; a C6 to C60 monocyclic or polycyclic aryl; a C2 to C60 monocyclic or polycyclic heteroaryl; —SiRR′R″; —P(═O)RR′; a C1 to C20 alkylamine; a C6 to C60 monocyclic or polycyclic arylamine; and a C2 to C60 monocyclic or polycyclic heteroarylamine, or being unsubstituted or substituted with a substituent to which two or more substituents selected among the exemplified substituents are linked.

The present application relates to the heterocyclic compound of Chemical Formula 1.

In an exemplary embodiment of the present application, the deuterium content of the heterocyclic compound of Chemical Formula 1 may be 0% to 100%.

In another exemplary embodiment, the deuterium content of the heterocyclic compound of Chemical Formula 1 may be more than 0% and 100% or less.

In still another exemplary embodiment, the deuterium content of the heterocyclic compound of Chemical Formula 1 may be 30% to 100%.

In yet another exemplary embodiment, the deuterium content of the heterocyclic compound of Chemical Formula 1 may be 50% to 100%.

In yet another exemplary embodiment, the deuterium content of the heterocyclic compound of Chemical Formula 1 may be 0%.

In yet another exemplary embodiment, the deuterium content of the heterocyclic compound of Chemical Formula 1 may be 100%.

In yet another exemplary embodiment, the deuterium content of the heterocyclic compound of Chemical Formula 1 may be 0% or 30% to 100%.

In yet another exemplary embodiment, the deuterium content of the heterocyclic compound of Chemical Formula 1 may be 0% or 50% to 100%.

Since the atomic mass of deuterium is twice as large as that of hydrogen, a compound comprising deuterium has lower zero-point energy and vibrational energy levels when compared to a general compound comprising hydrogen. In addition, the physicochemical characteristics such as the chemical bond length of deuterium are different from those of hydrogen, particularly the amplitude of the C-D bond is smaller than that of the C—H bond, so that the C-D bond has a stronger bond than the C—H bond because the van der Waals radius of deuterium is smaller than that of hydrogen.

Furthermore, a compound substituted with deuterium has a stable ground state energy, and as the bond length of deuterium and carbon becomes shorter, the molecular hardcore volume and the electrical polarizability are be reduced and the intermolecular interaction was weakened, so that when the compound substituted with deuterium is deposited, the volume of the thin film can be increased, and the increased volume can create an amorphous state which lowers the degree of crystallinity of the thin film.

The characteristics as described above may be effective for increasing the service life and driving characteristics of an organic light emitting device, and thermal stability may also be achieved.

In an exemplary embodiment of the present application, a compound in which deuterium is substituted through a reaction in which hydrogen is substituted with deuterium in the structure of the heterocyclic compound of Chemical Formula 1 can be prepared by various methods, and the substitution rate of deuterium may vary from 20 to 100% when a known deuterium substitution method is used. However, in general, when a deuterium substitution reaction is performed, it can be confirmed by 1H-NMR and LC/MS or GC/MS that the compound is substituted by 50 to 80%, and due to the optimization of deuterium substitution conditions, when a compound is substituted at a predetermined substitution rate of deuterium as in the heterocyclic compound of Chemical Formula 1 of the present application, an OLED device has a feature in which the service life characteristics are improved by 20% or more.

In an exemplary embodiment of the present application, Chemical Formula 1 may be represented by any one of the following Formulae 2 to 4.

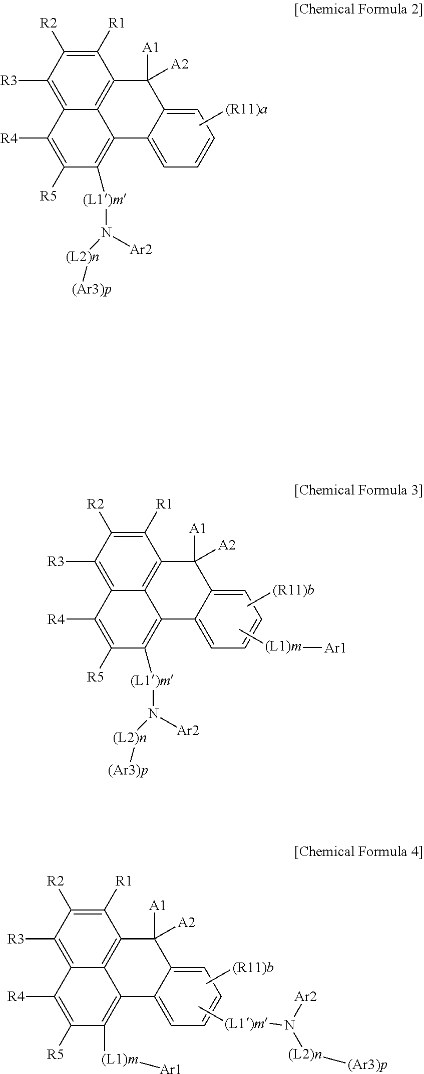

In Chemical Formulae 2 to 4,
the definitions of A1, A2, R1 to R5, L1, L1′, L2, Ar1 to Ar3, m, m′, n and p are the same as those in Chemical Formula 1,
R11 is hydrogen; or deuterium, and
a is an integer from 0 to 4, b is an integer from 0 to 3, and when a and b are each an integer of 2 or higher, substituents in the parenthesis are the same as or different from each other.

In an exemplary embodiment of the present application, A1 and A2 are the same as or different from each other, and may be each independently hydrogen; deuterium; a substituted or unsubstituted C1 to C60 alkyl group; a substituted or unsubstituted C6 to C60 aryl group; or a substituted or unsubstituted C2 to C60 heteroaryl group.

In another exemplary embodiment, A1 and A2 are the same as or different from each other, and may be each independently hydrogen; deuterium; a substituted or unsubstituted C1 to C40 alkyl group; a substituted or unsubstituted C6 to C40 aryl group; or a substituted or unsubstituted C2 to C40 heteroaryl group.

In still another exemplary embodiment, A1 and A2 are the same as or different from each other, and may be each independently hydrogen; deuterium; a C1 to C40 alkyl group which is unsubstituted or substituted with deuterium; a C6 to C40 aryl group which is unsubstituted or substituted with deuterium; or a C2 to C40 heteroaryl group which is unsubstituted or substituted with deuterium.

In yet another exemplary embodiment, A1 and A2 are the same as or different from each other, and may be each independently a C1 to C20 alkyl group which is unsubstituted or substituted with deuterium.

In yet another exemplary embodiment, A1 and A2 are the same as or different from each other, and may be each independently a C1 to C10 alkyl group which is unsubstituted or substituted with deuterium.

In yet another exemplary embodiment, A1 and A2 are the same as or different from each other, and may be each independently a C1 to C10 straight-chained alkyl group which is unsubstituted or substituted with deuterium; or a C3 to C10 branched alkyl group which is unsubstituted or substituted with deuterium.

In yet another exemplary embodiment, A1 and A2 are the same as or different from each other, and may be each independently a C1 to C10 straight-chained alkyl group which is unsubstituted or substituted with deuterium.

In yet another exemplary embodiment, A1 and A2 are the same as or different from each other, and may be each independently a methyl group; or a methyl group which is substituted with deuterium.

In an exemplary embodiment of the present application, R1 to R5 are the same as or different from each other, and may be each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; —P(═O)RR′; Chemical Formula 1-1 and Chemical Formula 1-2.

In another exemplary embodiment, R1 to R5 are the same as or different from each other, and may be each independently selected from the group consisting of hydrogen; deuterium; a substituted or unsubstituted C1 to C60 alkyl group; a substituted or unsubstituted C6 to C60 aryl group; a substituted or unsubstituted C2 to C60 heteroaryl group; SiRR′R″; and —P(═O)RR′.

In still another exemplary embodiment, R1 to R5 are the same as or different from each other, and may be each independently selected from the group consisting of hydrogen; deuterium; a substituted or unsubstituted C1 to C40 alkyl group; a substituted or unsubstituted C6 to C40 aryl group; or a substituted or unsubstituted C2 to C40 heteroaryl group.

In yet another exemplary embodiment, R1 to R5 are the same as or different from each other, and may be each independently selected from the group consisting of hydrogen; deuterium; a C1 to C40 alkyl group; a C6 to C40 aryl group; and a C2 to C40 heteroaryl group.

In yet another exemplary embodiment, R1 to R5 are the same as or different from each other, and may be each independently selected from the group consisting of hydrogen; deuterium; a C1 to C20 alkyl group; a C6 to C20 aryl group; and a C2 to C20 heteroaryl group.

In yet another exemplary embodiment, R1 to R5 are the same as or different from each other, and may be each independently hydrogen; or deuterium.

In an exemplary embodiment of the present application, R7 to R10 are the same as or different from each other, and may be each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; —P(═O)RR′; Chemical Formula 1-1 and Chemical Formula 1-2.

In another exemplary embodiment, R7 to R10 are the same as or different from each other, and may be each independently selected from the group consisting of hydrogen; deuterium; a substituted or unsubstituted C1 to C60 alkyl group; a substituted or unsubstituted C6 to C60 aryl group; a substituted or unsubstituted C2 to C60 heteroaryl group; Chemical Formula 1-1; and Chemical Formula 1-2.

In still another exemplary embodiment, R7 to R10 are the same as or different from each other, and may be each independently selected from the group consisting of hydrogen; deuterium; Chemical Formula 1-1; and Chemical Formula 1-2.

In an exemplary embodiment of the present application, R7 may be hydrogen; or deuterium.

In an exemplary embodiment of the present application, R6 of Chemical Formula 1 may be represented by Chemical Formula 1-1.

In an exemplary embodiment of the present application, R6 of Chemical Formula 1 may be represented by Chemical Formula 1-2.

In an exemplary embodiment of the present application, when R6 of Chemical Formula 1 is represented by Chemical Formula 1-1, R7 to R10 of Chemical Formula 1 may be hydrogen; or deuterium.

In an exemplary embodiment of the present application, when R6 of Chemical Formula 1 is represented by Chemical Formula 1-1, at least one of R7 to R10 of Chemical Formula 1 may be represented by Chemical Formula 1-2.

In an exemplary embodiment of the present application, when R6 of Chemical Formula 1 is represented by Chemical Formula 1-1, one of R7 to R10 of Chemical Formula 1 may be represented by Chemical Formula 1-2, and the others may be hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; or —P(═O)RR′.

In an exemplary embodiment of the present application, when R6 of Chemical Formula 1 is represented by Chemical Formula 1-1, one of R7 to R10 of Chemical Formula 1 may be represented by Chemical Formula 1-2, and the others may be hydrogen; or deuterium.

In an exemplary embodiment of the present application, when R6 of Chemical Formula 1 is represented by Chemical Formula 1-2, at least one of R7 to R10 of Chemical Formula 1 may be represented by Chemical Formula 1-1.

In an exemplary embodiment of the present application, when R6 of Chemical Formula 1 is represented by Chemical Formula 1-2, one of R7 to R10 of Chemical Formula 1 is represented by Chemical Formula 1-1, and the others may be hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; or —P(═O)RR′.

In an exemplary embodiment of the present application, when R6 of Chemical Formula 1 is represented by Chemical Formula 1-2, one of R7 to R10 of Chemical Formula 1 is represented by Chemical Formula 1-1, and the others may be hydrogen; or deuterium.

In an exemplary embodiment of the present application, L1, L1′ and L2 are the same as or different from each other, and may be each independently a direct bond; a substituted or unsubstituted C6 to C60 arylene group; or a substituted or unsubstituted C2 to C60 heteroarylene group.

In another exemplary embodiment, L1, L1′ and L2 are the same as or different from each other, and may be each independently a direct bond; a substituted or unsubstituted C6 to C40 arylene group; or a substituted or unsubstituted C2 to C40 heteroarylene group.

In still another exemplary embodiment, L1, L1′ and L2 are the same as or different from each other, and may be each independently a direct bond; or a substituted or unsubstituted C6 to C40 arylene group.

In yet another exemplary embodiment, L1, L1′ and L2 are the same as or different from each other, and may be each independently a direct bond; or a substituted or unsubstituted C6 to C30 arylene group.

In yet another exemplary embodiment, L1, L1′ and L2 are the same as or different from each other, and may be each independently a direct bond; or a substituted or unsubstituted C6 to C20 arylene group.

In yet another exemplary embodiment, L1, L1′ and L2 are the same as or different from each other, and may be each independently a direct bond; or a C6 to C20 arylene group.

In yet another exemplary embodiment, L1, L1′ and L2 are the same as or different from each other, and may be each independently a direct bond; or a monocyclic or polycyclic C6 to C20 arylene group.

In yet another exemplary embodiment, L1, L1′ and L2 are the same as or different from each other, and may be each independently a direct bond; a monocyclic C6 to C10 arylene group; or a polycyclic C10 to C20 arylene group.

In yet another exemplary embodiment, L1, L1′ and L2 are the same as or different from each other, and may be each independently a direct bond; a phenylene group; or a biphenylene group.

In an exemplary embodiment of the present application, the deuterium content of L1, L1′ and L2 may be each 0%, or 10% to 100%. In this case, the fact that the deuterium content of L1, L1′ and L2 is 100% may mean the case where hydrogens of L1, L1′ and L2 are all substituted with deuterium.

In an exemplary embodiment of the present application, the deuterium content of L1, L1′ and L2 may be 0% or 100%. In this case, the fact that the deuterium content of L1, L1′ and L2 is 100% may mean the case where substituents of L1, L1′ and L2 are all substituted with deuterium.

In an exemplary embodiment of the present application, Ar1 may be a substituted or unsubstituted C1 to C60 alkyl group; a substituted or unsubstituted C6 to C60 aryl group; or a substituted or unsubstituted C2 to C60 heteroaryl group.

In another exemplary embodiment, Ar1 may be a substituted or unsubstituted C1 to C40 alkyl group; a substituted or unsubstituted C6 to C40 aryl group; or a substituted or unsubstituted C2 to C40 heteroaryl group.

In still another exemplary embodiment, Ar1 may be a substituted or unsubstituted C1 to C30 alkyl group; a substituted or unsubstituted C6 to C30 aryl group; or a substituted or unsubstituted C2 to C30 heteroaryl group.

In yet another exemplary embodiment, Ar1 may be a C6 to C20 aryl group which is unsubstituted or substituted with a C1 to C20 alkyl group; or a C2 to C20 heteroaryl group.

In yet another exemplary embodiment, Ar1 may be a substituted or unsubstituted phenyl group; a substituted or unsubstituted biphenyl group; a substituted or unsubstituted naphthyl group; a substituted or unsubstituted anthracenyl group; a substituted or unsubstituted terphenyl group; a substituted or unsubstituted phenanthrenyl group; a substituted or unsubstituted dimethylfluorenyl group; a substituted or unsubstituted dibenzofuran group; or a substituted or unsubstituted dibenzothiophene group.

In yet another exemplary embodiment, Ar1 may be a phenyl group which is unsubstituted or substituted with deuterium; a biphenyl group which is unsubstituted or substituted with deuterium; a naphthyl group which is unsubstituted or substituted with deuterium; an anthracenyl group which is unsubstituted or substituted with deuterium; a terphenyl group which is unsubstituted or substituted with deuterium; a phenanthrenyl group which is unsubstituted or substituted with deuterium; a dimethylfluorenyl group which is unsubstituted or substituted with deuterium; a dibenzofuran group which is unsubstituted or substituted with deuterium; or a dibenzothiophene group which is unsubstituted or substituted with deuterium.

In yet another exemplary embodiment, Ar1 may be a substituted or unsubstituted phenyl group; a substituted or unsubstituted biphenyl group; a substituted or unsubstituted naphthyl group; a substituted or unsubstituted anthracenyl group; a substituted or unsubstituted terphenyl group; a substituted or unsubstituted dimethylfluorenyl group; a substituted or unsubstituted dibenzofuran group; or a substituted or unsubstituted dibenzothiophene group.

In yet another exemplary embodiment, Ar1 may be a phenyl group; a biphenyl group; a naphthyl group; an anthracenyl group; a terphenyl group; a dimethylfluorenyl group; a dibenzofuran group; or a dibenzothiophene group.

In an exemplary embodiment of the present application, the deuterium content of Ar1 may be 0%, or 10% to 100%. In this case, the fact that the deuterium content of Ar1 is 100% may mean the case where hydrogens of Ar1 are all substituted with deuterium.

In an exemplary embodiment of the present application, the deuterium content of Ar1 may be 0% or 100%. In this case, the fact that the deuterium content of Ar1 is 100% may mean the case where substituents of Ar1 are all substituted with deuterium.

In an exemplary embodiment of the present application, Ar1 may be represented by any one of the following structural formulae.

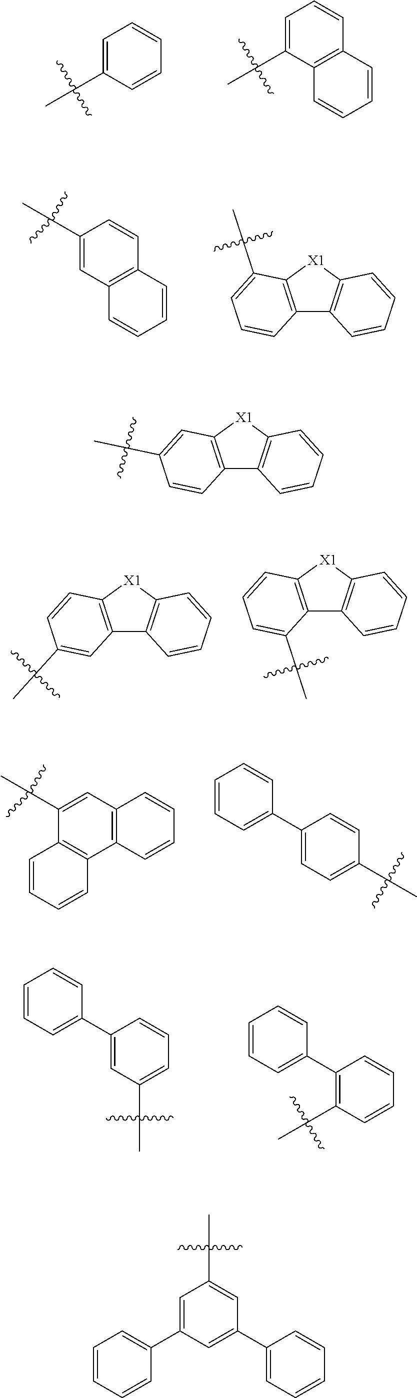

In the structural formulae,
X1 is O; S; or CRaRb,
Ra and Rb are the same as or different from each other, and are each independently a substituted or unsubstituted C1 to C60 alkyl group; or a substituted or unsubstituted C6 to C60 aryl group, and
the deuterium content of the structural formula is 0% or 100%.

In an exemplary embodiment of the present application, Ar2 and Ar3 are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another exemplary embodiment, Ar2 and Ar3 are the same as or different from each other, and may be each independently a substituted or unsubstituted C1 to C40 alkyl group; a substituted or unsubstituted C6 to C40 aryl group; or a substituted or unsubstituted C2 to C40 heteroaryl group.

In still another exemplary embodiment, Ar2 and Ar3 are the same as or different from each other, and may be each independently a C6 to C40 aryl group which is unsubstituted or substituted with one or more substituents selected from the group consisting of deuterium, a C1 to C40 alkyl group and a C6 to C40 aryl group; a spirobifluorenyl group; or a C2 to C40 heteroaryl group which is unsubstituted or substituted with deuterium.

In yet another exemplary embodiment, Ar2 and Ar3 are the same as or different from each other, and may be each independently a C6 to C30 aryl group which is unsubstituted or substituted with one or more substituents selected from the group consisting of deuterium, a C1 to C20 alkyl group and a C6 to C20 aryl group; a spirobifluorenyl group; or a C2 to C30 heteroaryl group which is unsubstituted or substituted with deuterium.

In yet another exemplary embodiment, Ar2 and Ar3 are the same as or different from each other, and may be each independently a phenyl group which is unsubstituted or substituted with deuterium; a biphenyl group which is unsubstituted or substituted with deuterium; a terphenyl group which is unsubstituted or substituted with deuterium; a dimethylfluorenyl group which is unsubstituted or substituted with deuterium; a diphenylfluorenyl group which is unsubstituted or substituted with deuterium; a spirobifluorenyl group which is unsubstituted or substituted with deuterium; a naphthyl group which is unsubstituted or substituted with deuterium; a triphenylenyl group which is unsubstituted or substituted with deuterium; an anthracenyl group which is unsubstituted or substituted with deuterium; a phenanthrenyl group which is unsubstituted or substituted with deuterium; a dibenzofuran group which is unsubstituted or substituted with deuterium; a dibenzothiophene group which is unsubstituted or substituted with deuterium; or a spiro[fluorene-9,9′-xanthene] group which is unsubstituted or substituted with deuterium.

In yet another exemplary embodiment, Ar2 and Ar3 are the same as or different from each other, and may be each independently a C6 to C40 aryl group which is unsubstituted or substituted with one or more substituents selected from the group consisting of a C1 to C40 alkyl group and a C6 to C40 aryl group; or a C2 to C40 heteroaryl group.

In yet another exemplary embodiment, Ar2 and Ar3 are the same as or different from each other, and may be each independently a C6 to C20 aryl group which is unsubstituted or substituted with one or more substituents selected from the group consisting of a C1 to C20 alkyl group and a C6 to C20 aryl group; or a C2 to C20 heteroaryl group.

In yet another exemplary embodiment, Ar2 and Ar3 are the same as or different from each other, and may be each independently a phenyl group; a biphenyl group; a terphenyl group; a dimethylfluorenyl group; a diphenylfluorenyl group; a spirobifluorenyl group; a naphthyl group; a triphenylenyl group; an anthracenyl group; a dibenzofuran group; a dibenzothiophene group; or a spiro[fluorene-9,9′-xanthene] group.

In an exemplary embodiment of the present application, the spiro[fluorene-9,9′-xanthene] group may have the following structure.

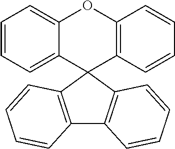 [SMILES: c1ccc2c(c1)Oc1ccccc1C21c2ccccc2-c2ccccc21]

In an exemplary embodiment of the present application, the terphenyl group is not limited as long as the terphenyl group has a structure in which three phenyl groups are linked. Specifically, the terphenyl group may satisfy the following structures, but is not limited thereto as long as the terphenyl group has a structure in which three phenyl ground are linked

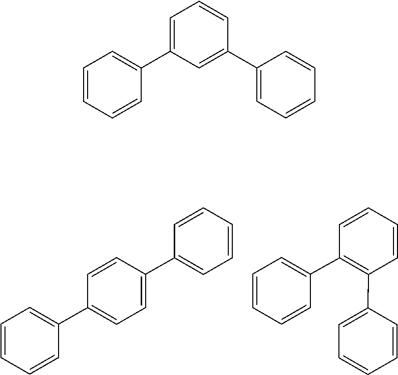

In an exemplary embodiment of the present application, the deuterium content of Ar2 and Ar3 may be each 0%, or 10% to 100%. In this case, the fact that the deuterium content of Ar2 and Ar3 is 100% may mean the case where hydrogens of Ar2 and Ar3 are all substituted with deuterium.

In an exemplary embodiment of the present application, the deuterium content of Ar2 and Ar3 may be 0% or 100%. In this case, the fact that the deuterium content of Ar2 and Ar3 is 100% may mean the case where substituents of Ar2 and Ar3 are all substituted with deuterium.

In an exemplary embodiment of the present application, the deuterium content of Chemical Formula 1-1 and Chemical Formula 1-2 may be each 0%, or 10% to 100%. In this case, the fact that the deuterium content of Chemical Formula 1-1 and Chemical Formula 1-2 is 100% may mean the case where all hydrogens of Chemical Formula 1-1 and Chemical Formula 1-2 are all substituted with deuterium.

In an exemplary embodiment of the present application, the deuterium content of Chemical Formula 1-1 and Chemical Formula 1-2 may be 0% or 100%. In this case, the fact that the deuterium content of Chemical Formula 1-1 and Chemical Formula 1-2 is 100% may mean the case where substituents of Chemical Formula 1-1 and Chemical Formula 1-2 are all substituted with deuterium.

In an exemplary embodiment of the present application, R, R′, and R″ are the same as or different from each other, and may be each independently a substituted or unsubstituted C1 to C60 alkyl group; a substituted or unsubstituted C6 to C60 aryl group; or a substituted or unsubstituted C2 to C60 heteroaryl group.

In another exemplary embodiment, R, R′, and R″ are the same as or different from each other, and may be each independently a substituted or unsubstituted C1 to C60 alkyl group; or a substituted or unsubstituted C6 to C60 aryl group.

In still another exemplary embodiment, R, R′, and R″ are the same as or different from each other, and may be each independently a C1 to C60 alkyl group; or a C6 to C60 aryl group.

In yet another exemplary embodiment, R, R′, and R″ are the same as or different from each other, and may be each independently a methyl group; or a phenyl group.

In yet another exemplary embodiment, R, R′, and R″ may be a phenyl group.

In an exemplary embodiment of the present application, the heterocyclic compound of Chemical Formula 1 may be represented by any one of the following compounds.

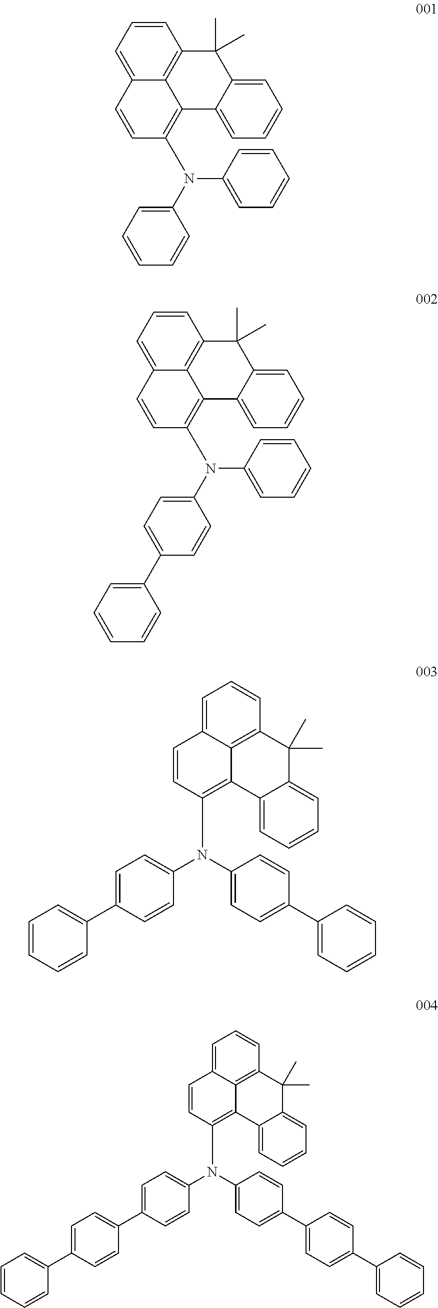 [SMILES: CC1(C)c2ccccc2-c2c(N(c3ccccc3)c3ccccc3)ccc3cccc1c23 ; CC1(C)c2ccccc2-c2c(N(c3ccccc3)c3ccc(-c4ccccc4)cc3)ccc3cccc1c23 ; CC1(C)c2ccccc2-c2c(N(c3ccc(-c4ccccc4)cc3)c3ccc(-c4ccccc4)cc3)ccc3cccc1c23 ; CC1(C)c2ccccc2-c2c(N(c3ccc(-c4ccc(-c5ccccc5)cc4)cc3)c3ccc(-c4ccc(-c5ccccc5)cc4)cc3)ccc3cccc1c23]

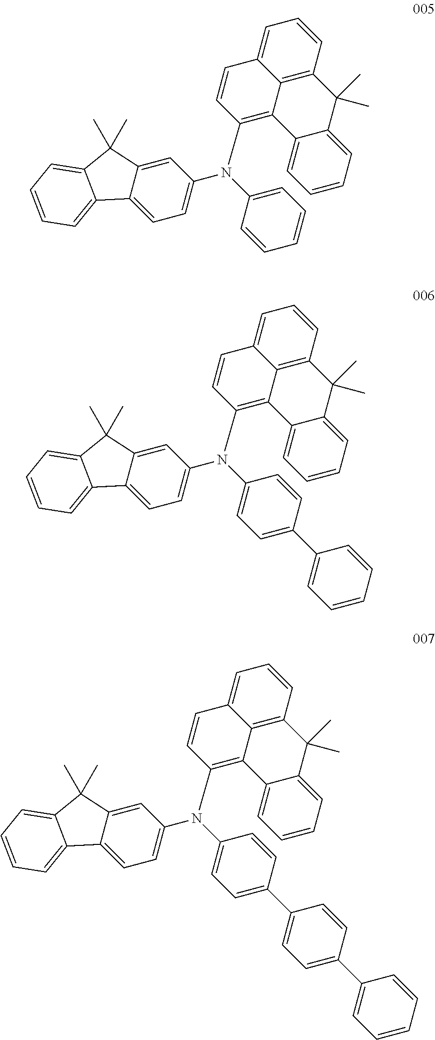 [SMILES: CC1(C)c2ccccc2-c2ccc(N(c3ccccc3)c3ccc4cccc5c4c3-c3ccccc3C5(C)C)cc21 ; CC1(C)c2ccccc2-c2ccc(N(c3ccc(-c4ccccc4)cc3)c3ccc4cccc5c4c3-c3ccccc3C5(C)C)cc21 ; CC1(C)c2ccccc2-c2ccc(N(c3ccc(-c4ccc(-c5ccccc5)cc4)cc3)c3ccc4cccc5c4c3-c3ccccc3C5(C)C)cc21]

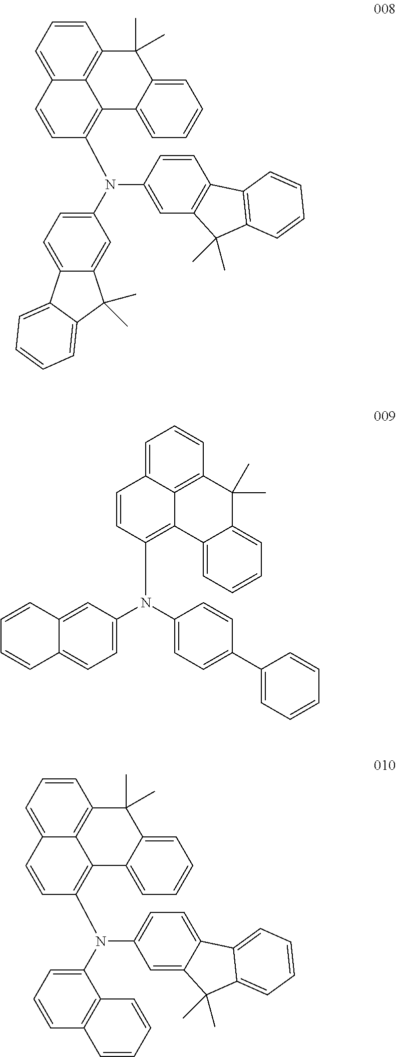 [SMILES: CC1(C)c2ccccc2-c2ccc(N(c3ccc4c(c3)C(C)(C)c3ccccc3-4)c3ccc4cccc5c4c3-c3ccccc3C5(C)C)cc21 ; CC1(C)c2ccccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccccc3C5(C)C)c3cccc4ccccc34)cc21]

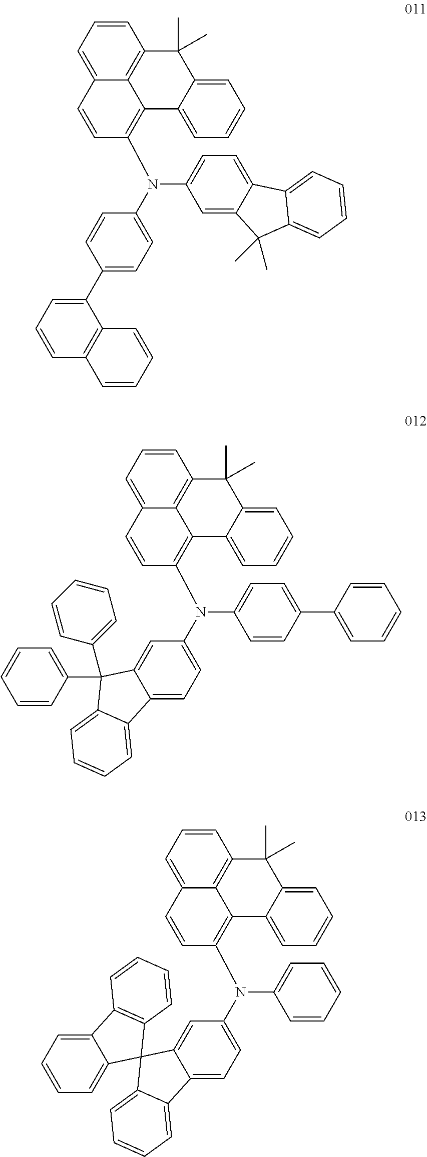 [SMILES: CC1(C)c2ccccc2-c2ccc(N(c3ccc(-c4cccc5ccccc45)cc3)c3ccc4cccc5c4c3-c3ccccc3C5(C)C)cc21 ; CC1(C)c2ccccc2-c2c(N(c3ccc(-c4ccccc4)cc3)c3ccc4c(c3)C(c3ccccc3)(c3ccccc3)c3ccccc3-4)ccc3cccc1c23 ; CC1(C)c2ccccc2-c2c(N(c3ccccc3)c3ccc4c(c3)C3(c5ccccc5-c5ccccc53)c3ccccc3-4)ccc3cccc1c23]

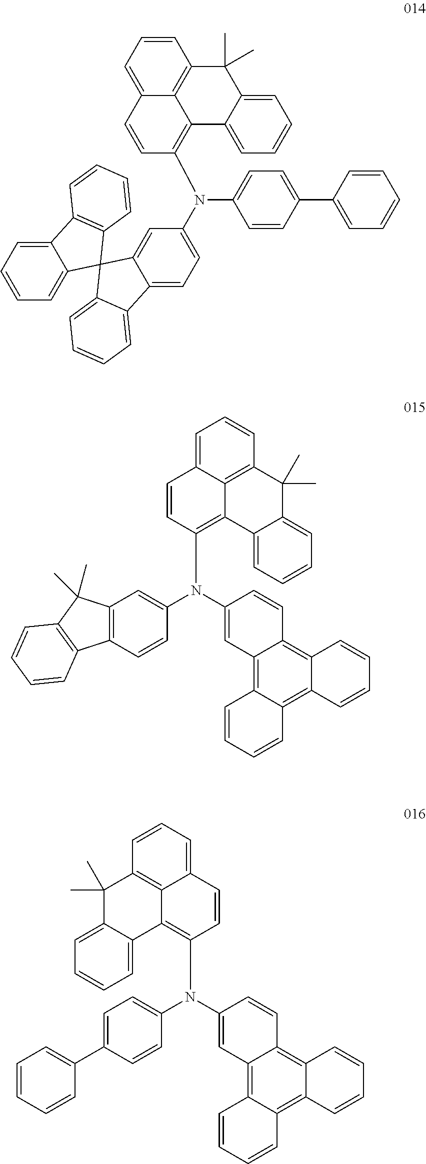 [SMILES: CC1(C)c2ccccc2-c2c(N(c3ccc(-c4ccccc4)cc3)c3ccc4c(c3)C3(c5ccccc5-c5ccccc53)c3ccccc3-4)ccc3cccc1c23 ; CC1(C)c2ccccc2-c2ccc(N(c3ccc4c5ccccc5c5ccccc5c4c3)c3ccc4cccc5c4c3-c3ccccc3C5(C)C)cc21 ; CC1(C)c2ccccc2-c2c(N(c3ccc(-c4ccccc4)cc3)c3ccc4c5ccccc5c5ccccc5c4c3)ccc3cccc1c23]

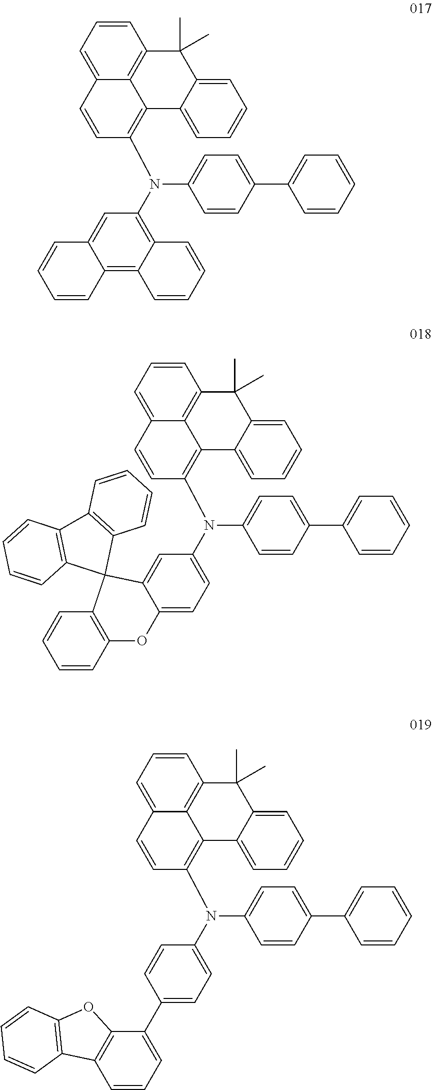 [SMILES: CC1(C)c2ccccc2-c2c(N(c3ccc(-c4ccccc4)cc3)c3cc4ccccc4c4ccccc34)ccc3cccc1c23 ; CC1(C)c2ccccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2ccccc2-c2c(N(c3ccc(-c4ccccc4)cc3)c3ccc(-c4cccc5c4oc4ccccc45)cc3)ccc3cccc1c23]

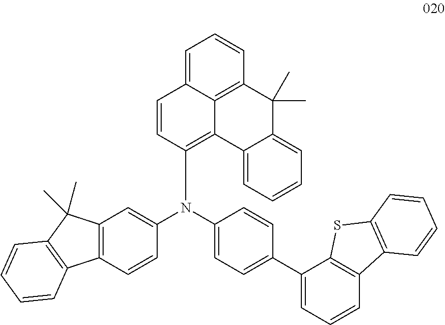 [SMILES: CC1(C)c2ccccc2-c2ccc(N(c3ccc(-c4cccc5c4sc4ccccc45)cc3)c3ccc4cccc5c4c3-c3ccccc3C5(C)C)cc21]

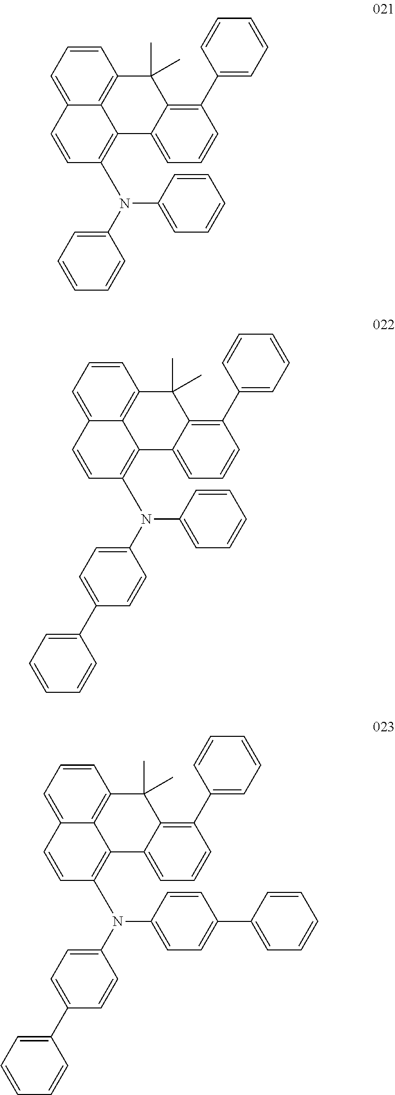 [SMILES: CC1(C)c2c(-c3ccccc3)cccc2-c2c(N(c3ccccc3)c3ccccc3)ccc3cccc1c23 ; CC1(C)c2c(-c3ccccc3)cccc2-c2c(N(c3ccccc3)c3ccc(-c4ccccc4)cc3)ccc3cccc1c23 ; CC1(C)c2c(-c3ccccc3)cccc2-c2c(N(c3ccc(-c4ccccc4)cc3)c3ccc(-c4ccccc4)cc3)ccc3cccc1c23]

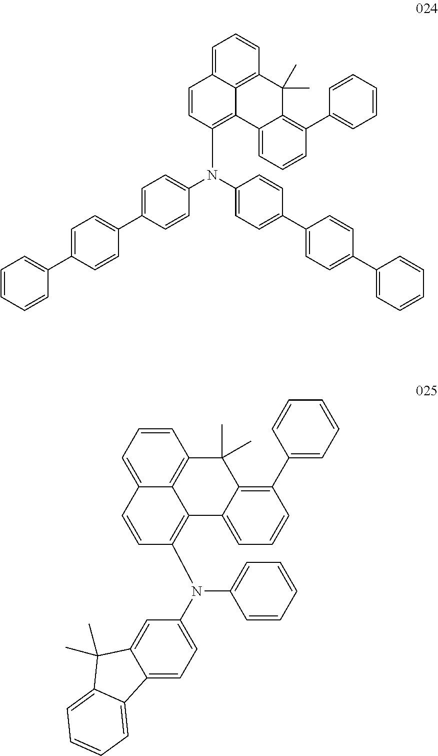 [SMILES: CC1(C)c2c(-c3ccccc3)cccc2-c2c(N(c3ccc(-c4ccc(-c5ccccc5)cc4)cc3)c3ccc(-c4ccc(-c5ccccc5)cc4)cc3)ccc3cccc1c23 ; CC1(C)c2ccccc2-c2ccc(N(c3ccccc3)c3ccc4cccc5c4c3-c3cccc(-c4ccccc4)c3C5(C)C)cc21]

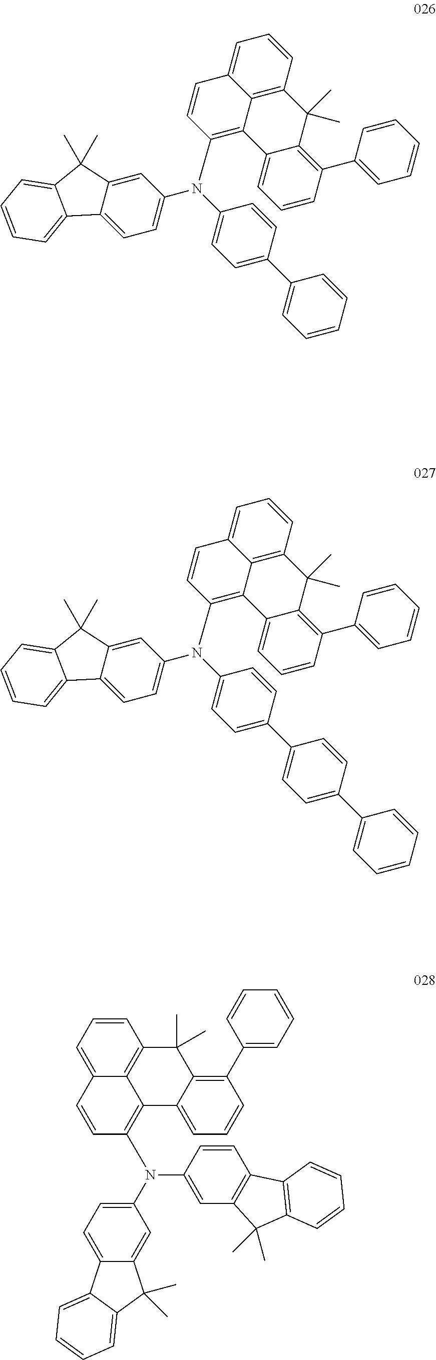 [SMILES: CC1(C)c2ccccc2-c2ccc(N(c3ccc(-c4ccccc4)cc3)c3ccc4cccc5c4c3-c3cccc(-c4ccccc4)c3C5(C)C)cc21 ; CC1(C)c2ccccc2-c2ccc(N(c3ccc(-c4ccc(-c5ccccc5)cc4)cc3)c3ccc4cccc5c4c3-c3cccc(-c4ccccc4)c3C5(C)C)cc21 ; CC1(C)c2ccccc2-c2ccc(N(c3ccc4c(c3)C(C)(C)c3ccccc3-4)c3ccc4cccc5c4c3-c3cccc(-c4ccccc4)c3C5(C)C)cc21]

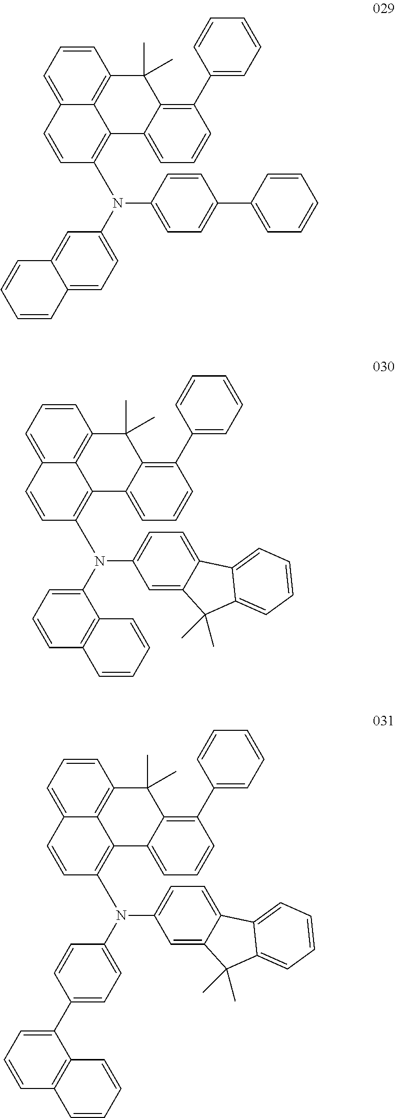 [SMILES: CC1(C)c2c(-c3ccccc3)cccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4ccccc4)c3C5(C)C)c3cccc4ccccc34)cc21 ; CC1(C)c2ccccc2-c2ccc(N(c3ccc(-c4cccc5ccccc45)cc3)c3ccc4cccc5c4c3-c3cccc(-c4ccccc4)c3C5(C)C)cc21]

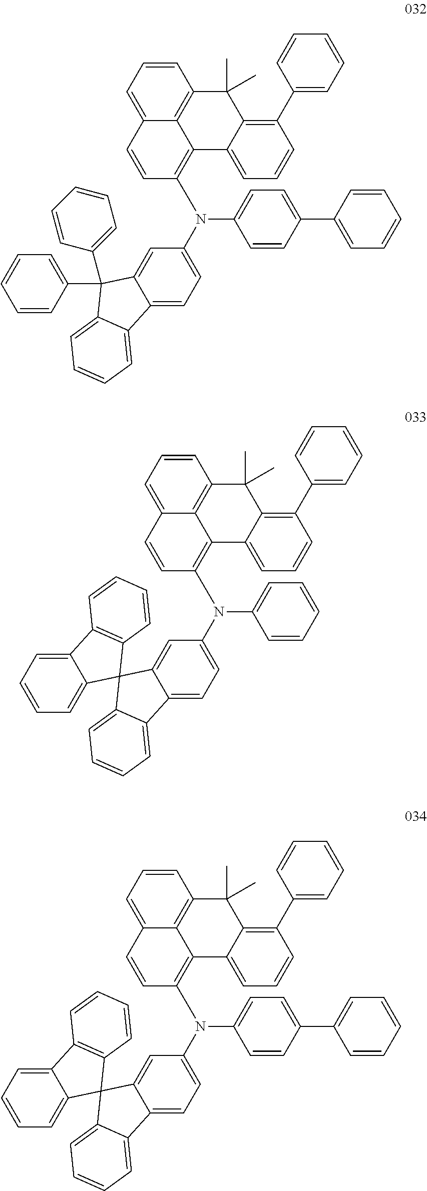 [SMILES: CC1(C)c2c(-c3ccccc3)cccc2-c2c(N(c3ccc(-c4ccccc4)cc3)c3ccc4c(c3)C(c3ccccc3)(c3ccccc3)c3ccccc3-4)ccc3cccc1c23 ; CC1(C)c2c(-c3ccccc3)cccc2-c2c(N(c3ccccc3)c3ccc4c(c3)C3(c5ccccc5-c5ccccc53)c3ccccc3-4)ccc3cccc1c23 ; CC1(C)c2c(-c3ccccc3)cccc2-c2c(N(c3ccc(-c4ccccc4)cc3)c3ccc4c(c3)C3(c5ccccc5-c5ccccc53)c3ccccc3-4)ccc3cccc1c23]

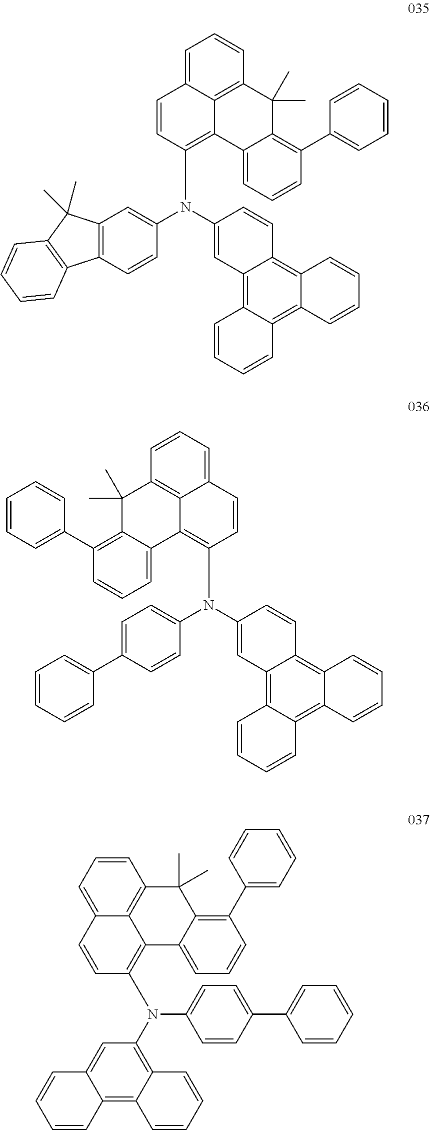 [SMILES: CC1(C)c2ccccc2-c2ccc(N(c3ccc4c5ccccc5c5ccccc5c4c3)c3ccc4cccc5c4c3-c3cccc(-c4ccccc4)c3C5(C)C)cc21 ; CC1(C)c2c(-c3ccccc3)cccc2-c2c(N(c3ccc(-c4ccccc4)cc3)c3ccc4c5ccccc5c5ccccc5c4c3)ccc3cccc1c23 ; CC1(C)c2c(-c3ccccc3)cccc2-c2c(N(c3ccc(-c4ccccc4)cc3)c3cc4ccccc4c4ccccc34)ccc3cccc1c23]

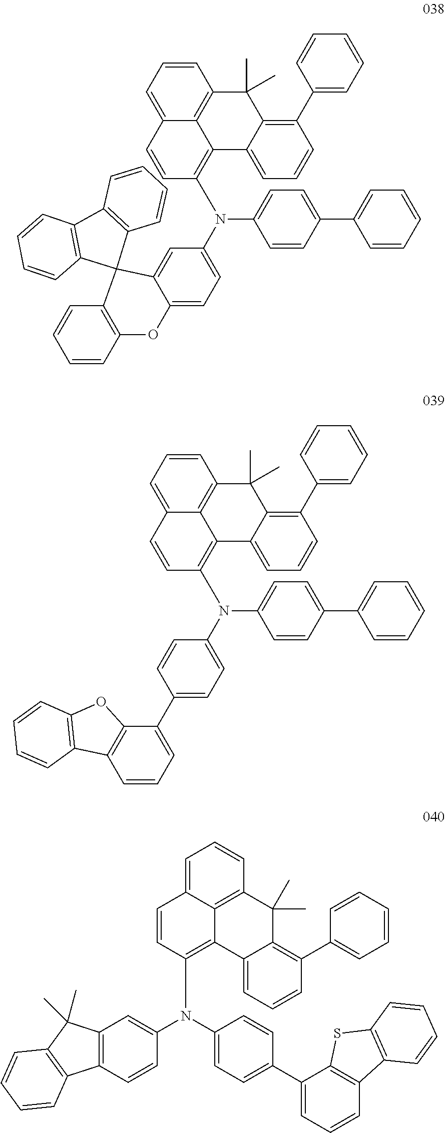 [SMILES: CC1(C)c2c(-c3ccccc3)cccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2c(-c3ccccc3)cccc2-c2c(N(c3ccc(-c4ccccc4)cc3)c3ccc(-c4cccc5c4oc4ccccc45)cc3)ccc3cccc1c23 ; CC1(C)c2ccccc2-c2ccc(N(c3ccc(-c4cccc5c4sc4ccccc45)cc3)c3ccc4cccc5c4c3-c3cccc(-c4ccccc4)c3C5(C)C)cc21]

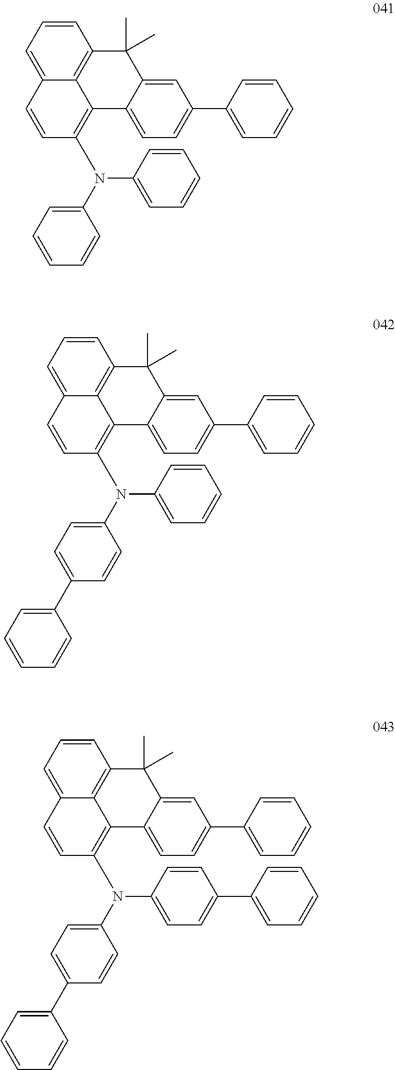 [SMILES: CC1(C)c2cc(-c3ccccc3)ccc2-c2c(N(c3ccccc3)c3ccccc3)ccc3cccc1c23 ; CC1(C)c2cc(-c3ccccc3)ccc2-c2c(N(c3ccccc3)c3ccc(-c4ccccc4)cc3)ccc3cccc1c23 ; CC1(C)c2cc(-c3ccccc3)ccc2-c2c(N(c3ccc(-c4ccccc4)cc3)c3ccc(-c4ccccc4)cc3)ccc3cccc1c23]

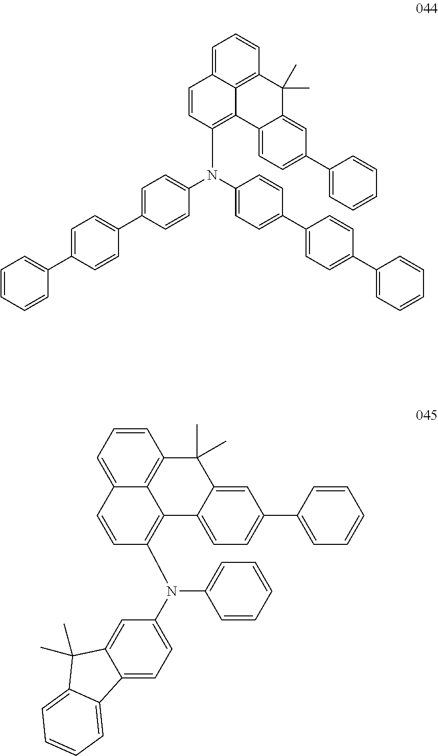 [SMILES: CC1(C)c2cc(-c3ccccc3)ccc2-c2c(N(c3ccc(-c4ccc(-c5ccccc5)cc4)cc3)c3ccc(-c4ccc(-c5ccccc5)cc4)cc3)ccc3cccc1c23 ; CC1(C)c2ccccc2-c2ccc(N(c3ccccc3)c3ccc4cccc5c4c3-c3ccc(-c4ccccc4)cc3C5(C)C)cc21]

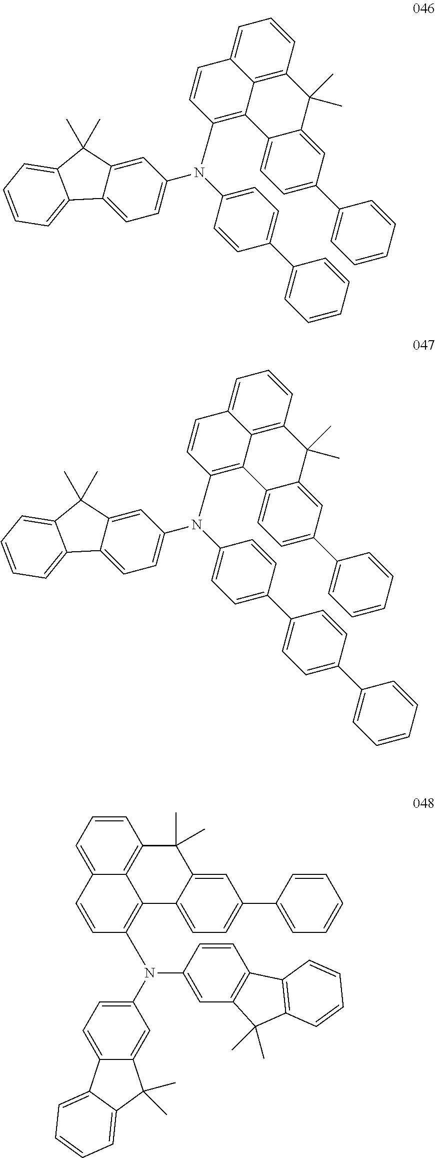 [SMILES: CC1(C)c2ccccc2-c2ccc(N(c3ccc(-c4ccccc4)cc3)c3ccc4cccc5c4c3-c3ccc(-c4ccccc4)cc3C5(C)C)cc21 ; CC1(C)c2ccccc2-c2ccc(N(c3ccc(-c4ccc(-c5ccccc5)cc4)cc3)c3ccc4cccc5c4c3-c3ccc(-c4ccccc4)cc3C5(C)C)cc21 ; CC1(C)c2ccccc2-c2ccc(N(c3ccc4c(c3)C(C)(C)c3ccccc3-4)c3ccc4cccc5c4c3-c3ccc(-c4ccccc4)cc3C5(C)C)cc21]

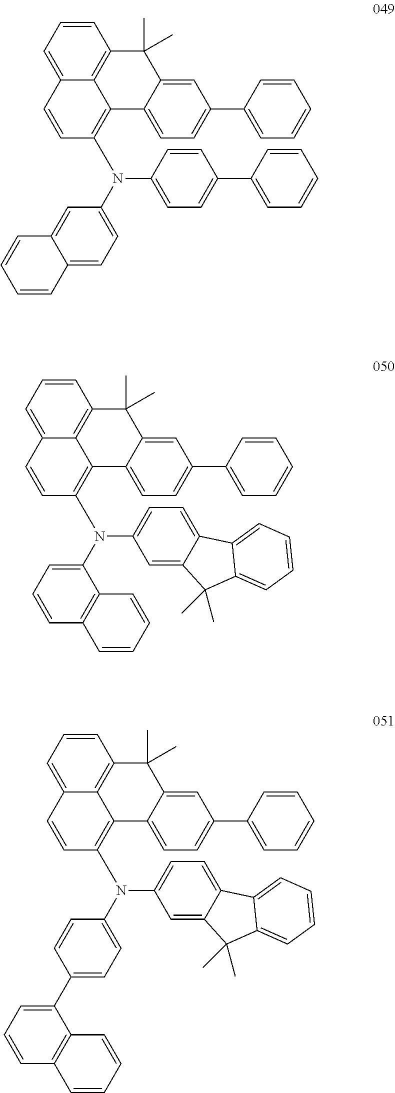 [SMILES: CC1(C)c2cc(-c3ccccc3)ccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccc(-c4ccccc4)cc3C5(C)C)c3cccc4ccccc34)cc21 ; CC1(C)c2ccccc2-c2ccc(N(c3ccc(-c4cccc5ccccc45)cc3)c3ccc4cccc5c4c3-c3ccc(-c4ccccc4)cc3C5(C)C)cc21]

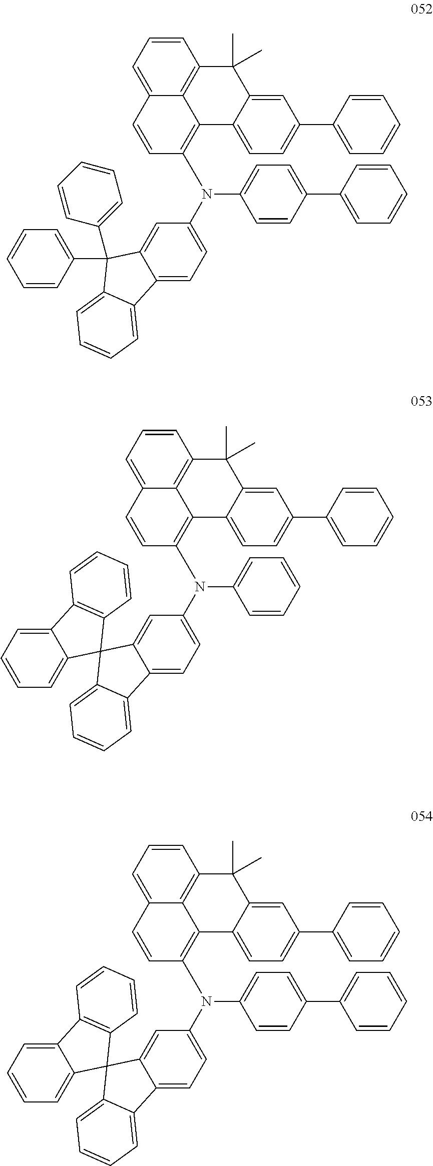 [SMILES: CC1(C)c2cc(-c3ccccc3)ccc2-c2c(N(c3ccc(-c4ccccc4)cc3)c3ccc4c(c3)C(c3ccccc3)(c3ccccc3)c3ccccc3-4)ccc3cccc1c23 ; CC1(C)c2cc(-c3ccccc3)ccc2-c2c(N(c3ccccc3)c3ccc4c(c3)C3(c5ccccc5-c5ccccc53)c3ccccc3-4)ccc3cccc1c23 ; CC1(C)c2cc(-c3ccccc3)ccc2-c2c(N(c3ccc(-c4ccccc4)cc3)c3ccc4c(c3)C3(c5ccccc5-c5ccccc53)c3ccccc3-4)ccc3cccc1c23]

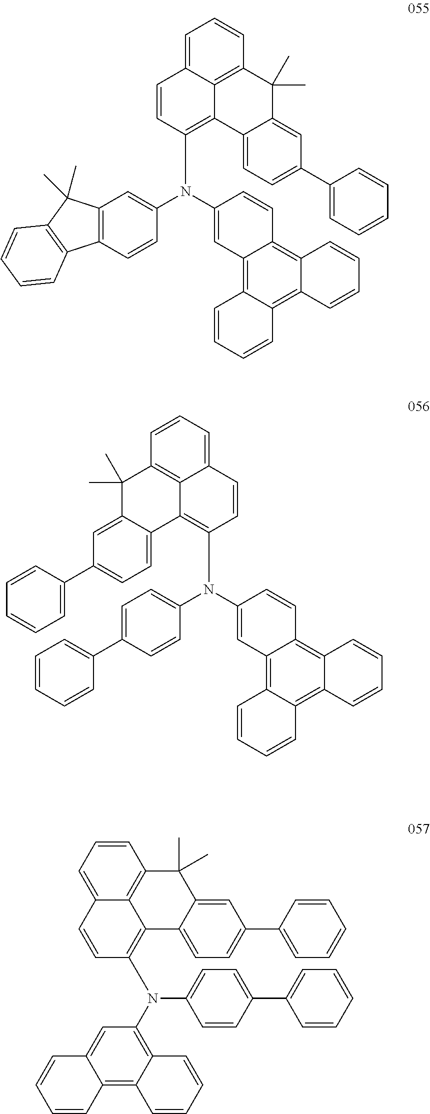 [SMILES: CC1(C)c2ccccc2-c2ccc(N(c3ccc4c5ccccc5c5ccccc5c4c3)c3ccc4cccc5c4c3-c3ccc(-c4ccccc4)cc3C5(C)C)cc21 ; CC1(C)c2cc(-c3ccccc3)ccc2-c2c(N(c3ccc(-c4ccccc4)cc3)c3ccc4c5ccccc5c5ccccc5c4c3)ccc3cccc1c23 ; CC1(C)c2cc(-c3ccccc3)ccc2-c2c(N(c3ccc(-c4ccccc4)cc3)c3cc4ccccc4c4ccccc34)ccc3cccc1c23]

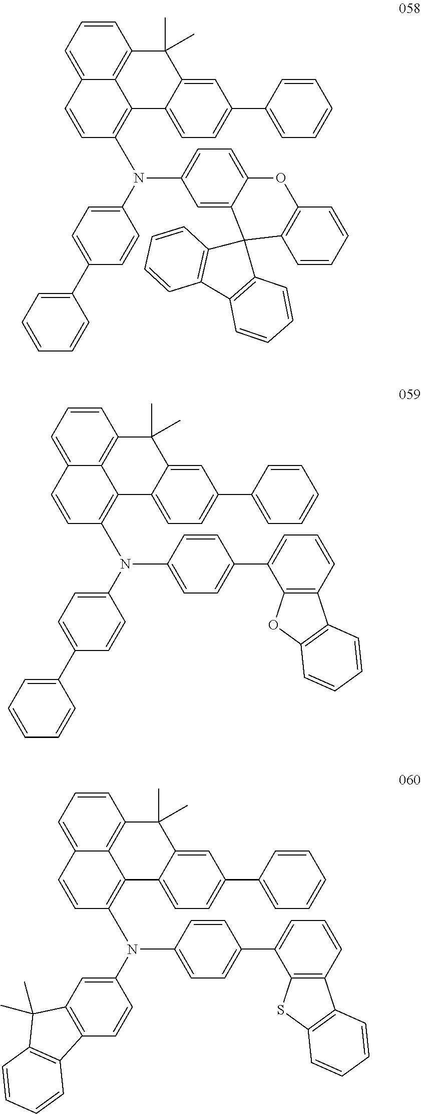 [SMILES: CC1(C)c2cc(-c3ccccc3)ccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2cc(-c3ccccc3)ccc2-c2c(N(c3ccc(-c4ccccc4)cc3)c3ccc(-c4cccc5c4oc4ccccc45)cc3)ccc3cccc1c23 ; CC1(C)c2ccccc2-c2ccc(N(c3ccc(-c4cccc5c4sc4ccccc45)cc3)c3ccc4cccc5c4c3-c3ccc(-c4ccccc4)cc3C5(C)C)cc21]

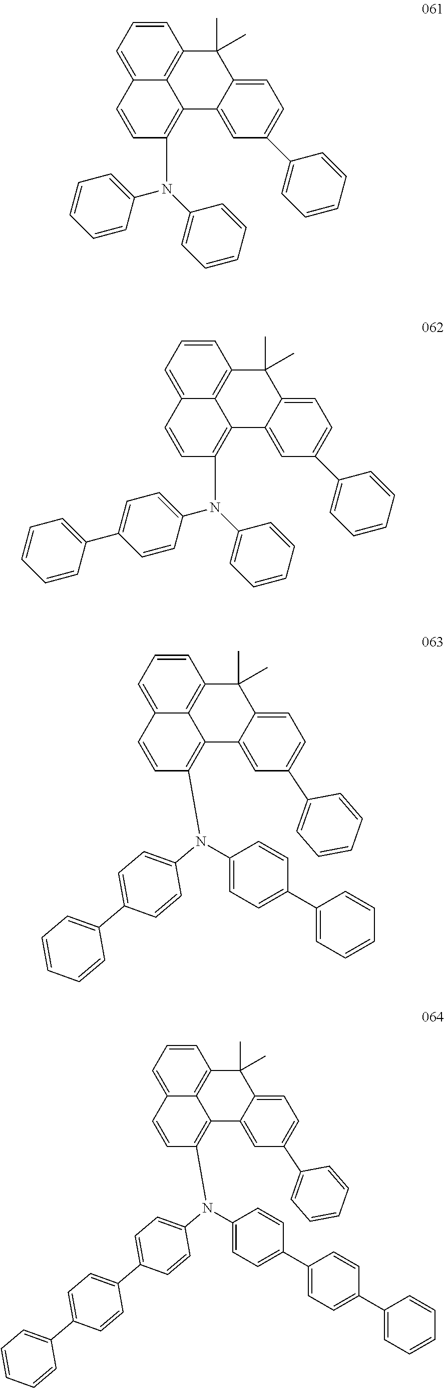 [SMILES: CC1(C)c2ccc(-c3ccccc3)cc2-c2c(N(c3ccccc3)c3ccccc3)ccc3cccc1c23 ; CC1(C)c2ccc(-c3ccccc3)cc2-c2c(N(c3ccccc3)c3ccc(-c4ccccc4)cc3)ccc3cccc1c23 ; CC1(C)c2ccc(-c3ccccc3)cc2-c2c(N(c3ccc(-c4ccccc4)cc3)c3ccc(-c4ccccc4)cc3)ccc3cccc1c23 ; CC1(C)c2ccc(-c3ccccc3)cc2-c2c(N(c3ccc(-c4ccc(-c5ccccc5)cc4)cc3)c3ccc(-c4ccc(-c5ccccc5)cc4)cc3)ccc3cccc1c23]

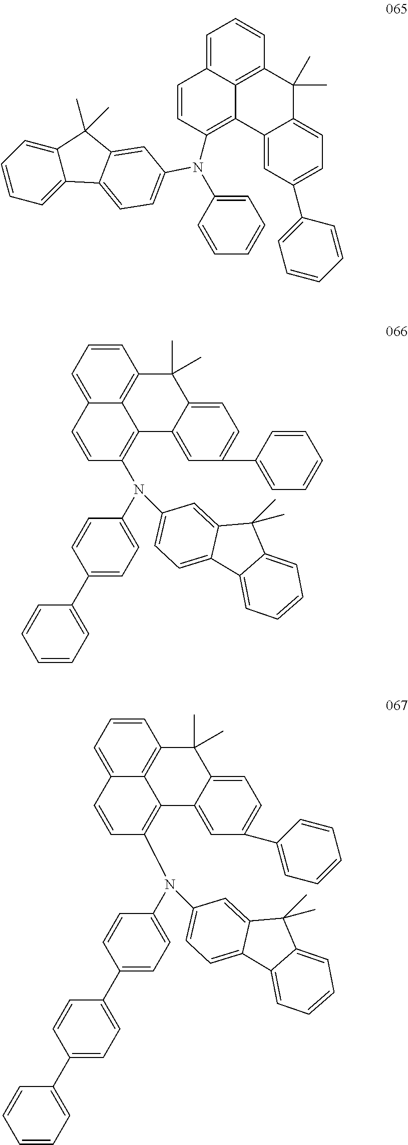 [SMILES: CC1(C)c2ccccc2-c2ccc(N(c3ccccc3)c3ccc4cccc5c4c3-c3cc(-c4ccccc4)ccc3C5(C)C)cc21 ; CC1(C)c2ccccc2-c2ccc(N(c3ccc(-c4ccccc4)cc3)c3ccc4cccc5c4c3-c3cc(-c4ccccc4)ccc3C5(C)C)cc21 ; CC1(C)c2ccccc2-c2ccc(N(c3ccc(-c4ccc(-c5ccccc5)cc4)cc3)c3ccc4cccc5c4c3-c3cc(-c4ccccc4)ccc3C5(C)C)cc21]

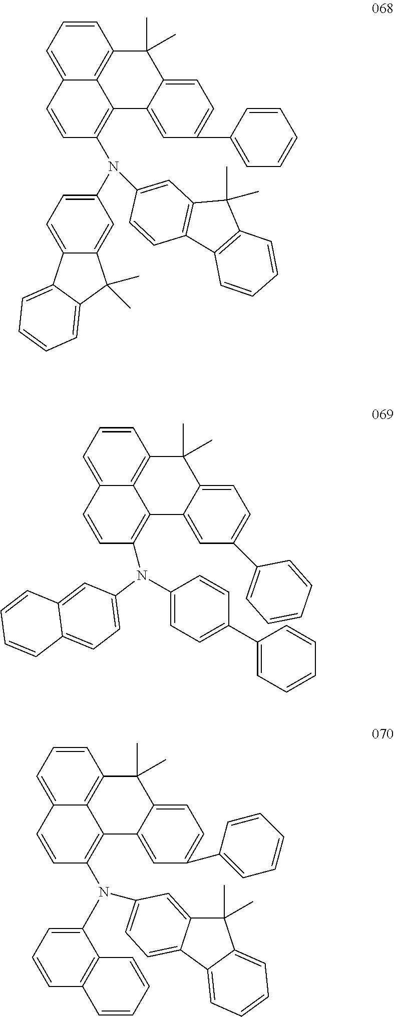 [SMILES: CC1(C)c2ccccc2-c2ccc(N(c3ccc4c(c3)C(C)(C)c3ccccc3-4)c3ccc4cccc5c4c3-c3cc(-c4ccccc4)ccc3C5(C)C)cc21 ; CC1(C)c2ccc(-c3ccccc3)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cc(-c4ccccc4)ccc3C5(C)C)c3cccc4ccccc34)cc21]

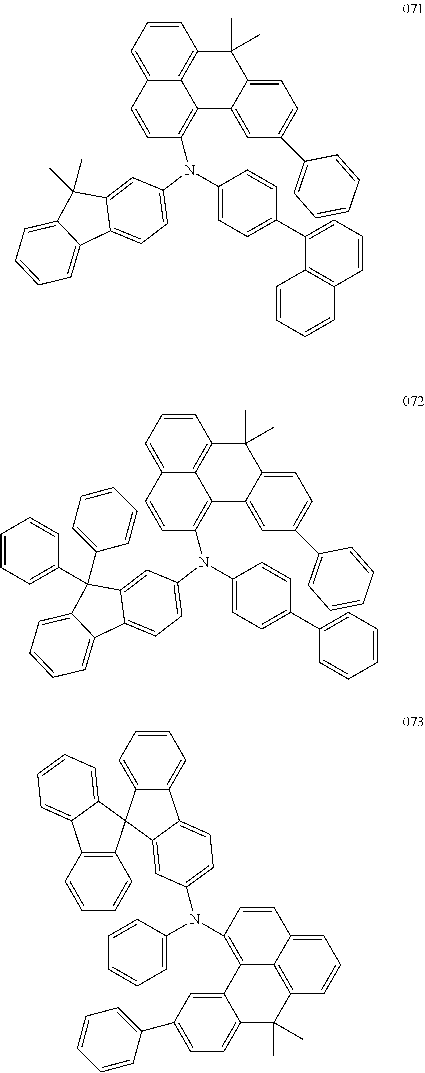 [SMILES: CC1(C)c2ccccc2-c2ccc(N(c3ccc(-c4cccc5ccccc45)cc3)c3ccc4cccc5c4c3-c3cc(-c4ccccc4)ccc3C5(C)C)cc21 ; CC1(C)c2ccc(-c3ccccc3)cc2-c2c(N(c3ccc(-c4ccccc4)cc3)c3ccc4c(c3)C(c3ccccc3)(c3ccccc3)c3ccccc3-4)ccc3cccc1c23 ; CC1(C)c2ccc(-c3ccccc3)cc2-c2c(N(c3ccccc3)c3ccc4c(c3)C3(c5ccccc5-c5ccccc53)c3ccccc3-4)ccc3cccc1c23]

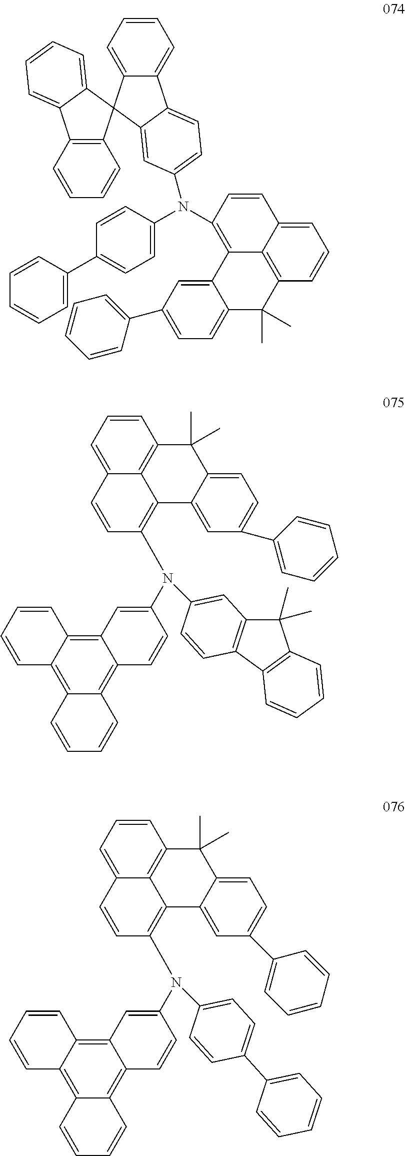 [SMILES: CC1(C)c2ccc(-c3ccccc3)cc2-c2c(N(c3ccc(-c4ccccc4)cc3)c3ccc4c(c3)C3(c5ccccc5-c5ccccc53)c3ccccc3-4)ccc3cccc1c23 ; CC1(C)c2ccccc2-c2ccc(N(c3ccc4c5ccccc5c5ccccc5c4c3)c3ccc4cccc5c4c3-c3cc(-c4ccccc4)ccc3C5(C)C)cc21 ; CC1(C)c2ccc(-c3ccccc3)cc2-c2c(N(c3ccc(-c4ccccc4)cc3)c3ccc4c5ccccc5c5ccccc5c4c3)ccc3cccc1c23]

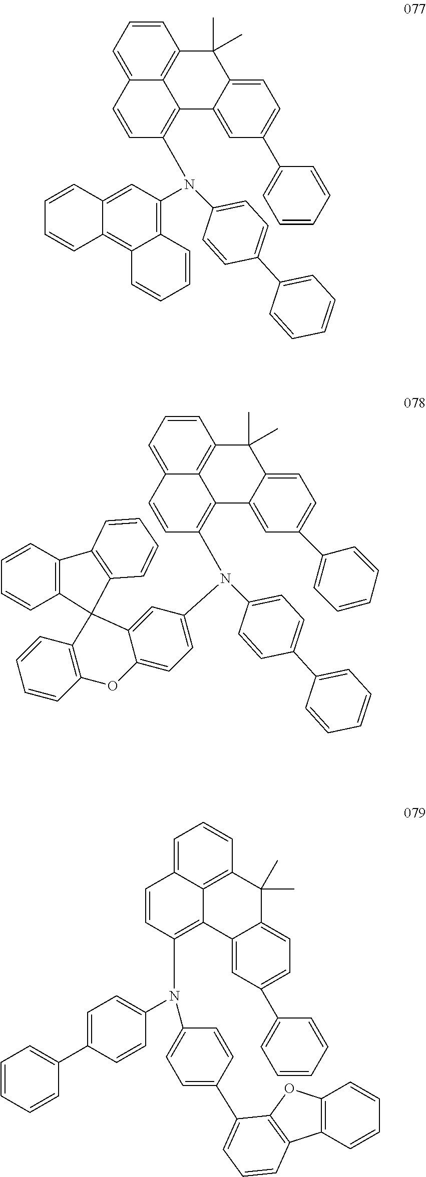 [SMILES: CC1(C)c2ccc(-c3ccccc3)cc2-c2c(N(c3ccc(-c4ccccc4)cc3)c3cc4ccccc4c4ccccc34)ccc3cccc1c23 ; CC1(C)c2ccc(-c3ccccc3)cc2-c2c(N(c3ccc(-c4ccccc4)cc3)c3ccc4c(c3)C3(c5ccccc5O4)c4ccccc4-c4ccccc43)ccc3cccc1c23 ; CC1(C)c2ccc(-c3ccccc3)cc2-c2c(N(c3ccc(-c4ccccc4)cc3)c3ccc(-c4cccc5c4oc4ccccc45)cc3)ccc3cccc1c23]

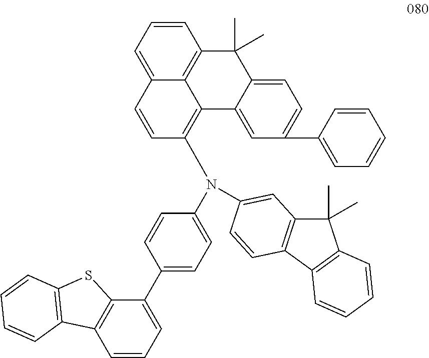 [SMILES: CC1(C)c2ccccc2-c2ccc(N(c3ccc(-c4cccc5c4sc4ccccc45)cc3)c3ccc4cccc5c4c3-c3cc(-c4ccccc4)ccc3C5(C)C)cc21]

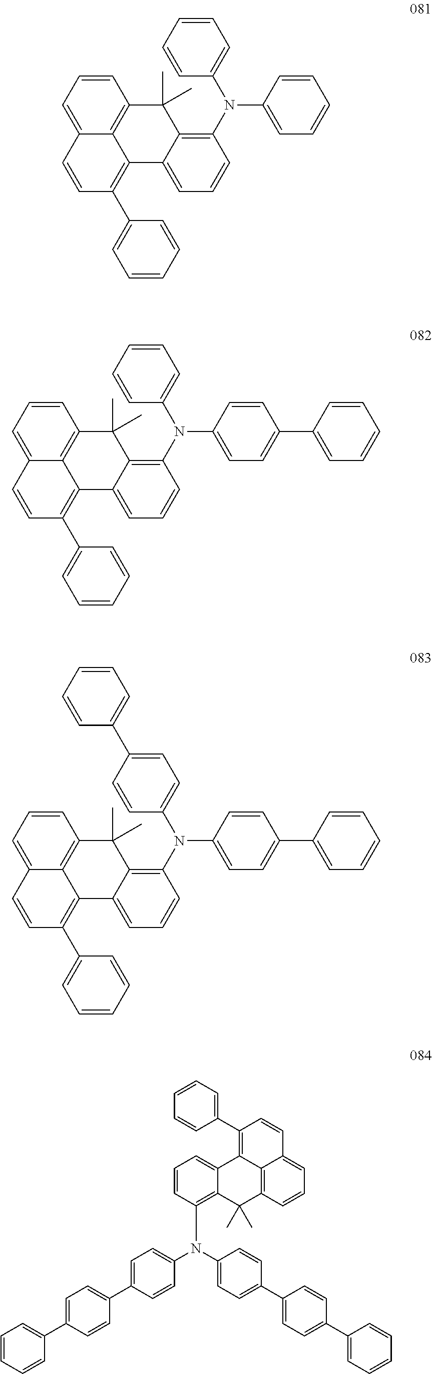 [SMILES: CC1(C)c2c(cccc2N(c2ccccc2)c2ccccc2)-c2c(-c3ccccc3)ccc3cccc1c23 ; CC1(C)c2c(cccc2N(c2ccccc2)c2ccc(-c3ccccc3)cc2)-c2c(-c3ccccc3)ccc3cccc1c23 ; CC1(C)c2c(cccc2N(c2ccc(-c3ccccc3)cc2)c2ccc(-c3ccccc3)cc2)-c2c(-c3ccccc3)ccc3cccc1c23 ; CC1(C)c2c(cccc2N(c2ccc(-c3ccc(-c4ccccc4)cc3)cc2)c2ccc(-c3ccc(-c4ccccc4)cc3)cc2)-c2c(-c3ccccc3)ccc3cccc1c23]

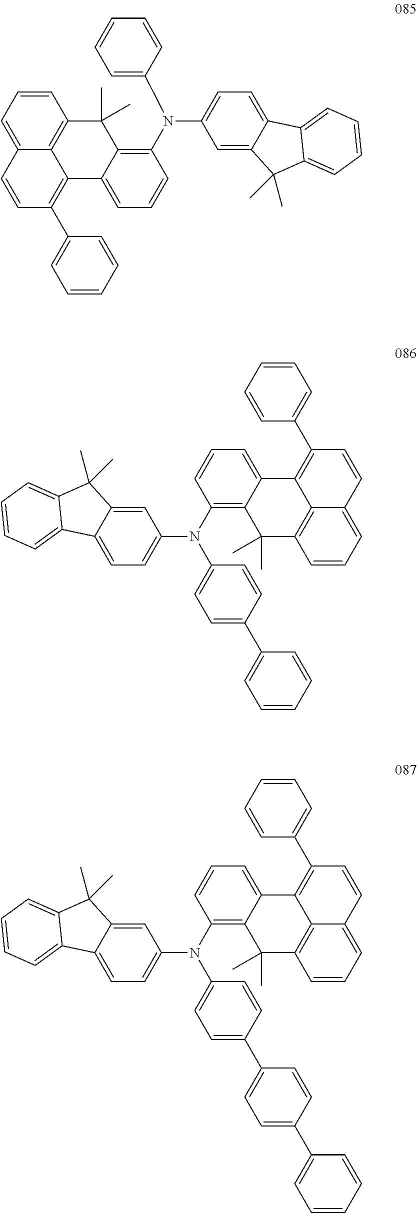 [SMILES: CC1(C)c2ccccc2-c2ccc(N(c3ccccc3)c3cccc4c3C(C)(C)c3cccc5ccc(-c6ccccc6)c-4c35)cc21 ; CC1(C)c2ccccc2-c2ccc(N(c3ccc(-c4ccccc4)cc3)c3cccc4c3C(C)(C)c3cccc5ccc(-c6ccccc6)c-4c35)cc21 ; CC1(C)c2ccccc2-c2ccc(N(c3ccc(-c4ccc(-c5ccccc5)cc4)cc3)c3cccc4c3C(C)(C)c3cccc5ccc(-c6ccccc6)c-4c35)cc21]

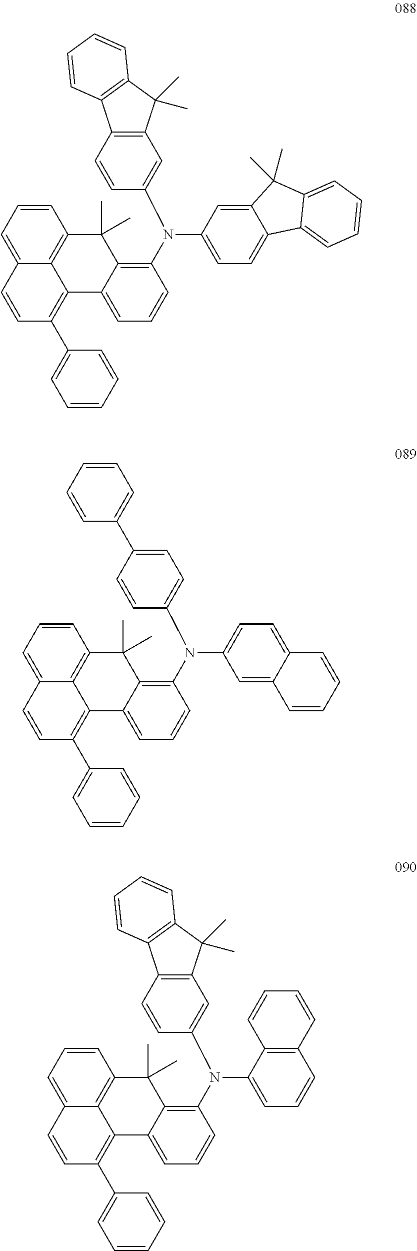 [SMILES: CC1(C)c2ccccc2-c2ccc(N(c3ccc4c(c3)C(C)(C)c3ccccc3-4)c3cccc4c3C(C)(C)c3cccc5ccc(-c6ccccc6)c-4c35)cc21 ; CC1(C)c2c(cccc2N(c2ccc(-c3ccccc3)cc2)c2ccc3ccccc3c2)-c2c(-c3ccccc3)ccc3cccc1c23 ; CC1(C)c2ccccc2-c2ccc(N(c3cccc4c3C(C)(C)c3cccc5ccc(-c6ccccc6)c-4c35)c3cccc4ccccc34)cc21]

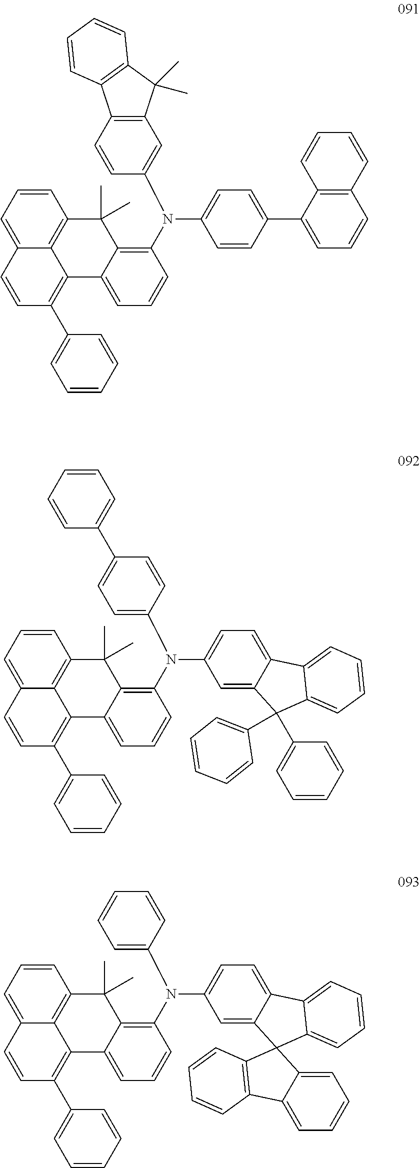 [SMILES: CC1(C)c2ccccc2-c2ccc(N(c3ccc(-c4cccc5ccccc45)cc3)c3cccc4c3C(C)(C)c3cccc5ccc(-c6ccccc6)c-4c35)cc21 ; CC1(C)c2c(cccc2N(c2ccc(-c3ccccc3)cc2)c2ccc3c(c2)C(c2ccccc2)(c2ccccc2)c2ccccc2-3)-c2c(-c3ccccc3)ccc3cccc1c23 ; CC1(C)c2c(cccc2N(c2ccccc2)c2ccc3c(c2)C2(c4ccccc4-c4ccccc42)c2ccccc2-3)-c2c(-c3ccccc3)ccc3cccc1c23]

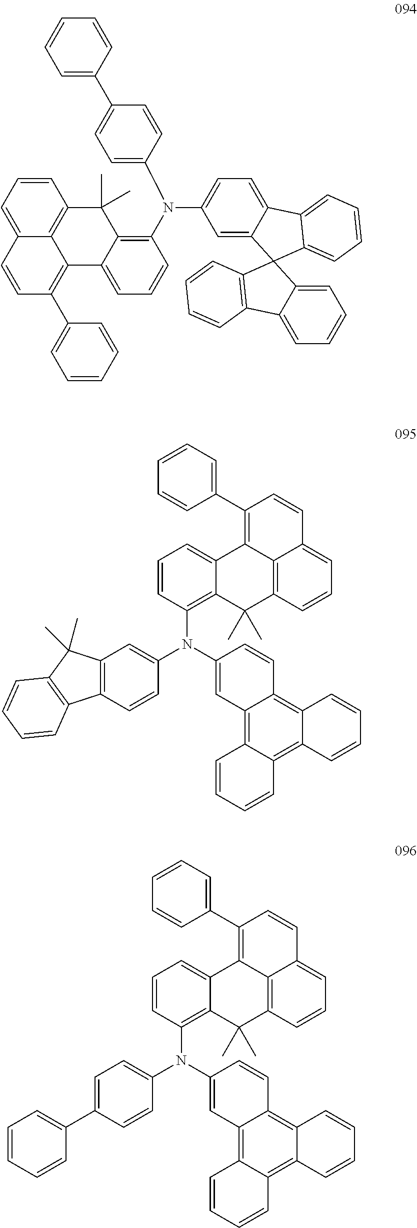 [SMILES: CC1(C)c2c(cccc2N(c2ccc(-c3ccccc3)cc2)c2ccc3c(c2)C2(c4ccccc4-c4ccccc42)c2ccccc2-3)-c2c(-c3ccccc3)ccc3cccc1c23 ; CC1(C)c2ccccc2-c2ccc(N(c3ccc4c5ccccc5c5ccccc5c4c3)c3cccc4c3C(C)(C)c3cccc5ccc(-c6ccccc6)c-4c35)cc21 ; CC1(C)c2c(cccc2N(c2ccc(-c3ccccc3)cc2)c2ccc3c4ccccc4c4ccccc4c3c2)-c2c(-c3ccccc3)ccc3cccc1c23]

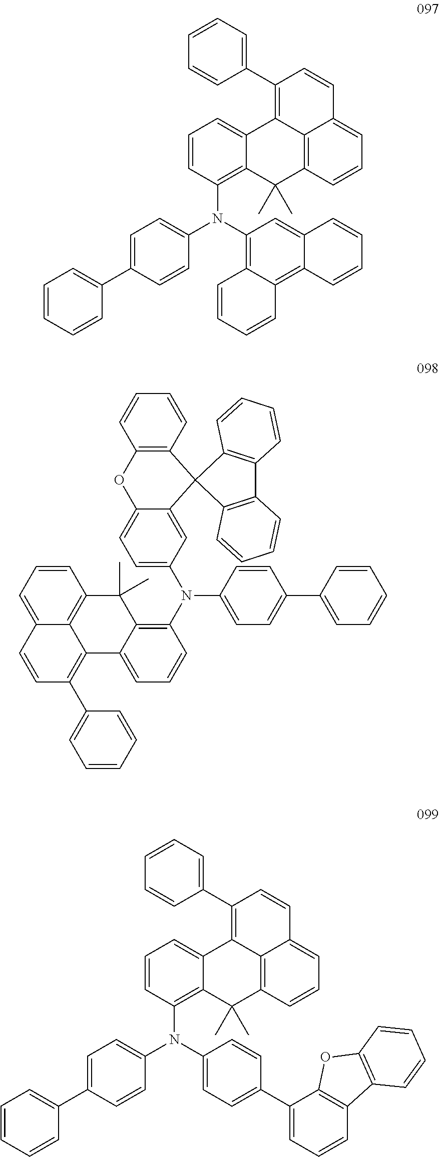 [SMILES: CC1(C)c2c(cccc2N(c2ccc(-c3ccccc3)cc2)c2cc3ccccc3c3ccccc23)-c2c(-c3ccccc3)ccc3cccc1c23 ; CC1(C)c2c(cccc2N(c2ccc(-c3ccccc3)cc2)c2ccc3c(c2)C2(c4ccccc4O3)c3ccccc3-c3ccccc32)-c2c(-c3ccccc3)ccc3cccc1c23 ; CC1(C)c2c(cccc2N(c2ccc(-c3ccccc3)cc2)c2ccc(-c3cccc4c3oc3ccccc34)cc2)-c2c(-c3ccccc3)ccc3cccc1c23]

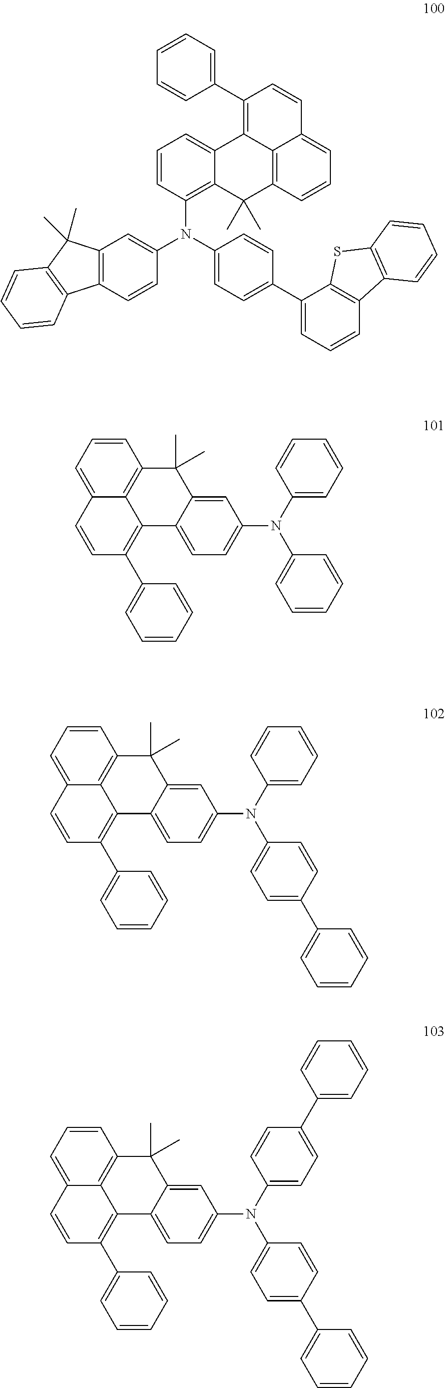 [SMILES: CC1(C)c2ccccc2-c2ccc(N(c3ccc(-c4cccc5c4sc4ccccc45)cc3)c3cccc4c3C(C)(C)c3cccc5ccc(-c6ccccc6)c-4c35)cc21 ; CC1(C)c2cc(N(c3ccccc3)c3ccccc3)ccc2-c2c(-c3ccccc3)ccc3cccc1c23 ; CC1(C)c2cc(N(c3ccccc3)c3ccc(-c4ccccc4)cc3)ccc2-c2c(-c3ccccc3)ccc3cccc1c23 ; CC1(C)c2cc(N(c3ccc(-c4ccccc4)cc3)c3ccc(-c4ccccc4)cc3)ccc2-c2c(-c3ccccc3)ccc3cccc1c23]

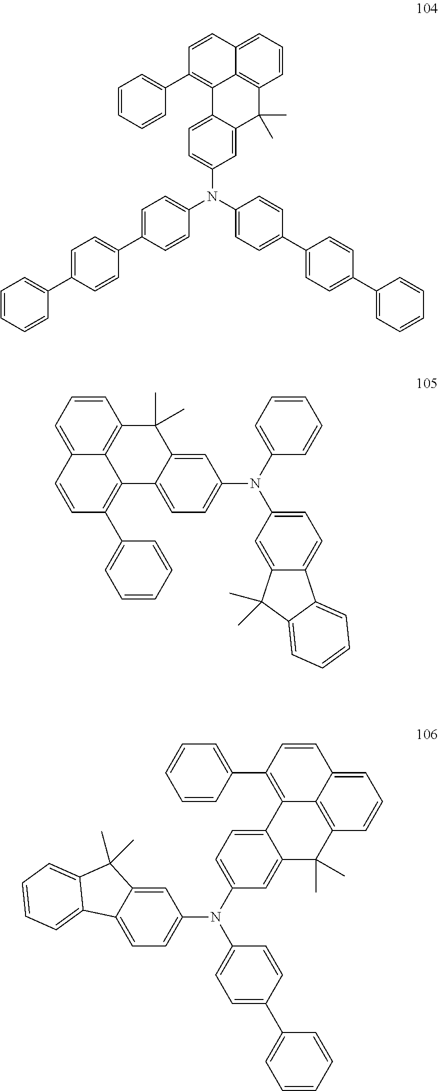 [SMILES: CC1(C)c2cc(N(c3ccc(-c4ccc(-c5ccccc5)cc4)cc3)c3ccc(-c4ccc(-c5ccccc5)cc4)cc3)ccc2-c2c(-c3ccccc3)ccc3cccc1c23 ; CC1(C)c2ccccc2-c2ccc(N(c3ccccc3)c3ccc4c(c3)C(C)(C)c3cccc5ccc(-c6ccccc6)c-4c35)cc21 ; CC1(C)c2ccccc2-c2ccc(N(c3ccc(-c4ccccc4)cc3)c3ccc4c(c3)C(C)(C)c3cccc5ccc(-c6ccccc6)c-4c35)cc21]

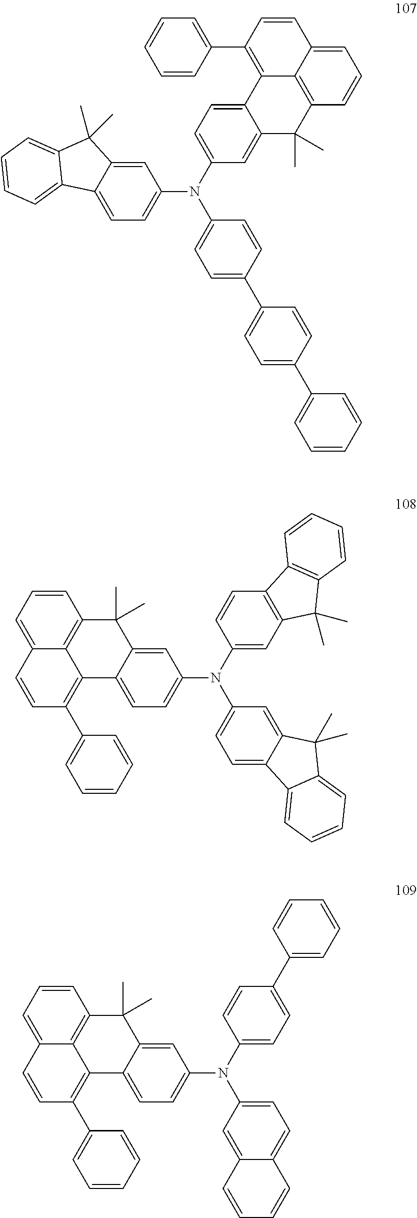 [SMILES: CC1(C)c2ccccc2-c2ccc(N(c3ccc(-c4ccc(-c5ccccc5)cc4)cc3)c3ccc4c(c3)C(C)(C)c3cccc5ccc(-c6ccccc6)c-4c35)cc21 ; CC1(C)c2ccccc2-c2ccc(N(c3ccc4c(c3)C(C)(C)c3ccccc3-4)c3ccc4c(c3)C(C)(C)c3cccc5ccc(-c6ccccc6)c-4c35)cc21 ; CC1(C)c2cc(N(c3ccc(-c4ccccc4)cc3)c3ccc4ccccc4c3)ccc2-c2c(-c3ccccc3)ccc3cccc1c23]

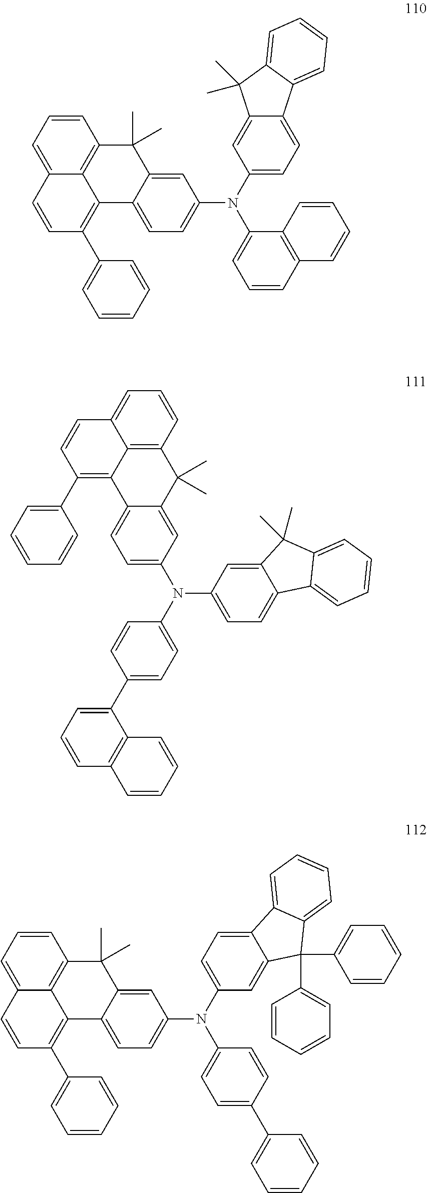 [SMILES: CC1(C)c2ccccc2-c2ccc(N(c3ccc4c(c3)C(C)(C)c3cccc5ccc(-c6ccccc6)c-4c35)c3cccc4ccccc34)cc21 ; CC1(C)c2ccccc2-c2ccc(N(c3ccc(-c4cccc5ccccc45)cc3)c3ccc4c(c3)C(C)(C)c3cccc5ccc(-c6ccccc6)c-4c35)cc21 ; CC1(C)c2cc(N(c3ccc(-c4ccccc4)cc3)c3ccc4c(c3)C(c3ccccc3)(c3ccccc3)c3ccccc3-4)ccc2-c2c(-c3ccccc3)ccc3cccc1c23]

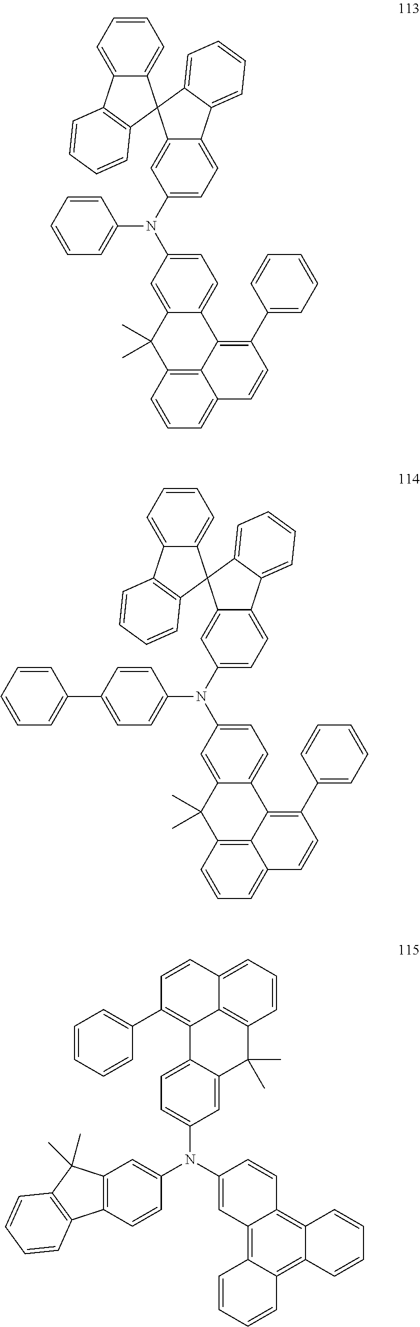 [SMILES: CC1(C)c2cc(N(c3ccccc3)c3ccc4c(c3)C3(c5ccccc5-c5ccccc53)c3ccccc3-4)ccc2-c2c(-c3ccccc3)ccc3cccc1c23 ; CC1(C)c2cc(N(c3ccc(-c4ccccc4)cc3)c3ccc4c(c3)C3(c5ccccc5-c5ccccc53)c3ccccc3-4)ccc2-c2c(-c3ccccc3)ccc3cccc1c23 ; CC1(C)c2ccccc2-c2ccc(N(c3ccc4c(c3)C(C)(C)c3cccc5ccc(-c6ccccc6)c-4c35)c3ccc4c5ccccc5c5ccccc5c4c3)cc21]

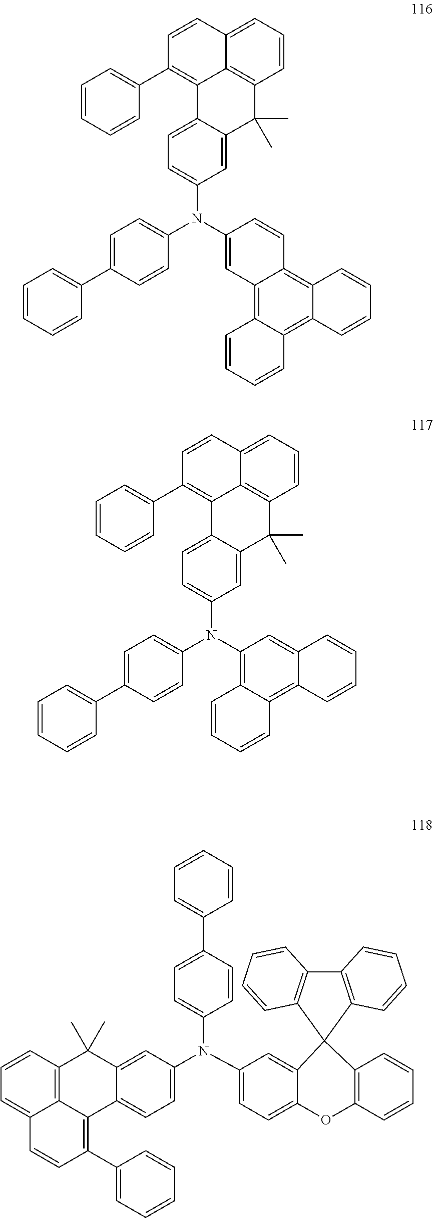 [SMILES: CC1(C)c2cc(N(c3ccc(-c4ccccc4)cc3)c3ccc4c5ccccc5c5ccccc5c4c3)ccc2-c2c(-c3ccccc3)ccc3cccc1c23 ; CC1(C)c2cc(N(c3ccc(-c4ccccc4)cc3)c3cc4ccccc4c4ccccc34)ccc2-c2c(-c3ccccc3)ccc3cccc1c23 ; CC1(C)c2cc(N(c3ccc(-c4ccccc4)cc3)c3ccc4c(c3)C3(c5ccccc5O4)c4ccccc4-c4ccccc43)ccc2-c2c(-c3ccccc3)ccc3cccc1c23]

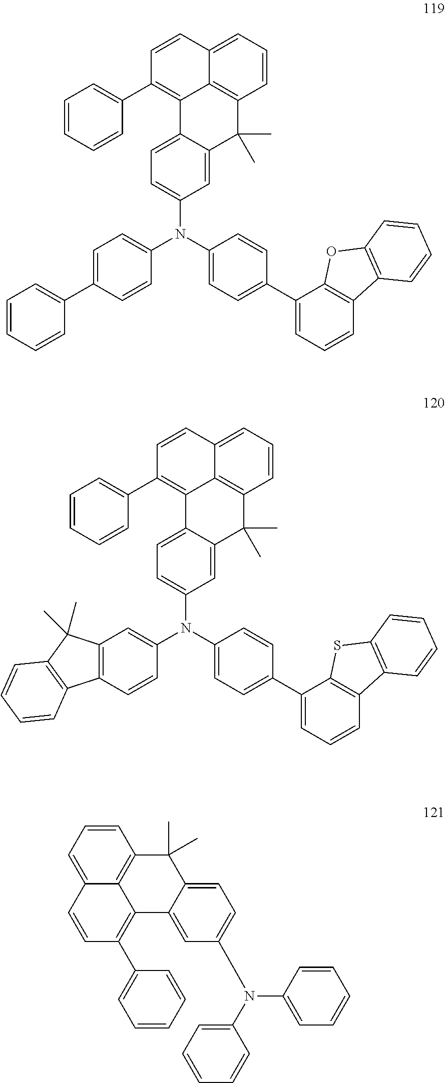 [SMILES: CC1(C)c2cc(N(c3ccc(-c4ccccc4)cc3)c3ccc(-c4cccc5c4oc4ccccc45)cc3)ccc2-c2c(-c3ccccc3)ccc3cccc1c23 ; CC1(C)c2ccccc2-c2ccc(N(c3ccc(-c4cccc5c4sc4ccccc45)cc3)c3ccc4c(c3)C(C)(C)c3cccc5ccc(-c6ccccc6)c-4c35)cc21 ; CC1(C)c2ccc(N(c3ccccc3)c3ccccc3)cc2-c2c(-c3ccccc3)ccc3cccc1c23]

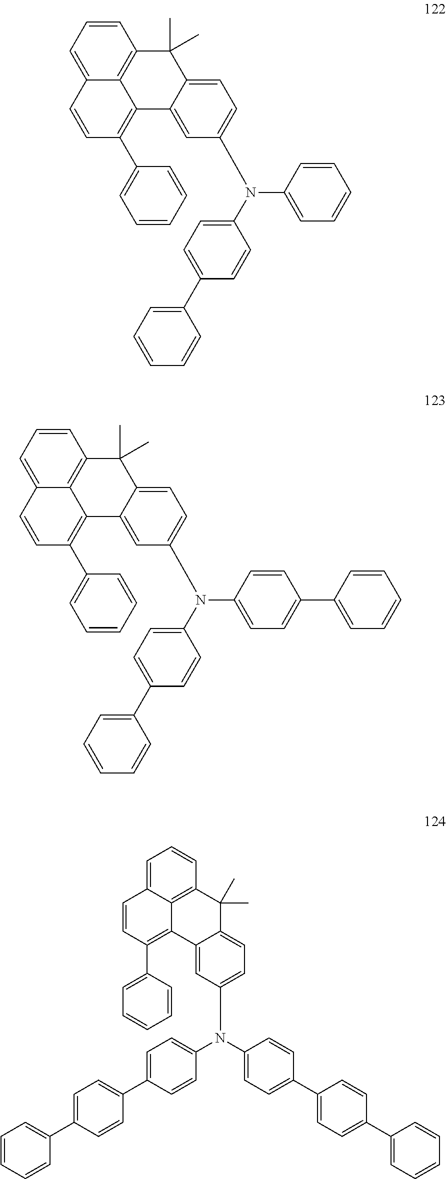 [SMILES: CC1(C)c2ccc(N(c3ccccc3)c3ccc(-c4ccccc4)cc3)cc2-c2c(-c3ccccc3)ccc3cccc1c23 ; CC1(C)c2ccc(N(c3ccc(-c4ccccc4)cc3)c3ccc(-c4ccccc4)cc3)cc2-c2c(-c3ccccc3)ccc3cccc1c23 ; CC1(C)c2ccc(N(c3ccc(-c4ccc(-c5ccccc5)cc4)cc3)c3ccc(-c4ccc(-c5ccccc5)cc4)cc3)cc2-c2c(-c3ccccc3)ccc3cccc1c23]

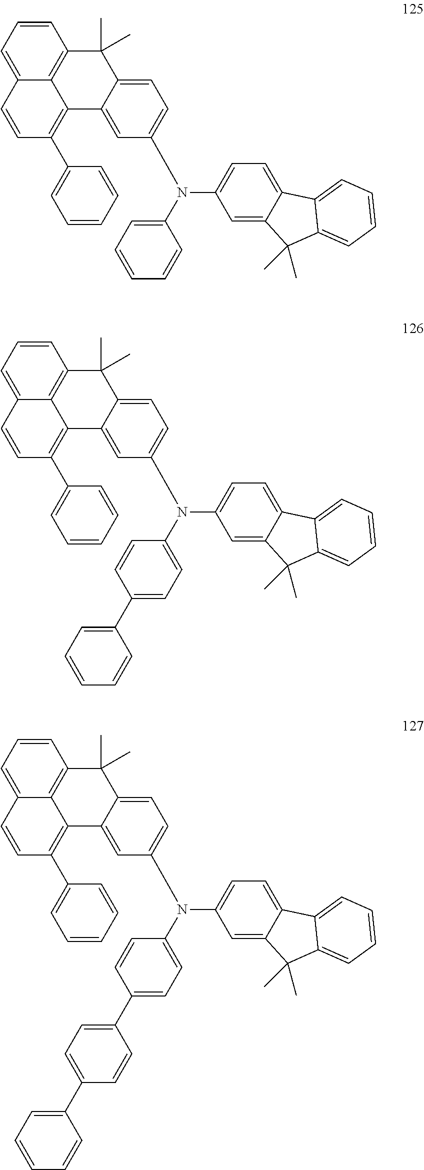 [SMILES: CC1(C)c2ccccc2-c2ccc(N(c3ccccc3)c3ccc4c(c3)-c3c(-c5ccccc5)ccc5cccc(c35)C4(C)C)cc21 ; CC1(C)c2ccccc2-c2ccc(N(c3ccc(-c4ccccc4)cc3)c3ccc4c(c3)-c3c(-c5ccccc5)ccc5cccc(c35)C4(C)C)cc21 ; CC1(C)c2ccccc2-c2ccc(N(c3ccc(-c4ccc(-c5ccccc5)cc4)cc3)c3ccc4c(c3)-c3c(-c5ccccc5)ccc5cccc(c35)C4(C)C)cc21]

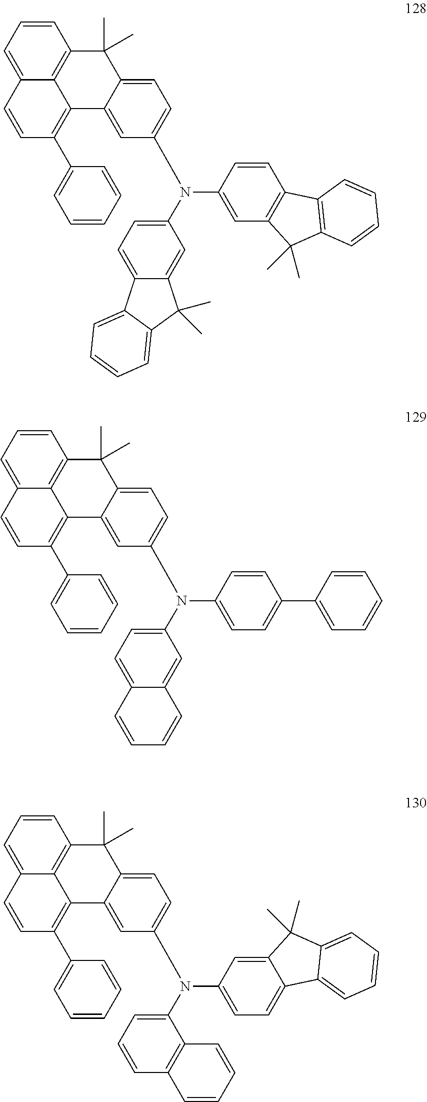 [SMILES: CC1(C)c2ccccc2-c2ccc(N(c3ccc4c(c3)-c3c(-c5ccccc5)ccc5cccc(c35)C4(C)C)c3ccc4c(c3)C(C)(C)c3ccccc3-4)cc21 ; CC1(C)c2ccc(N(c3ccc(-c4ccccc4)cc3)c3ccc4ccccc4c3)cc2-c2c(-c3ccccc3)ccc3cccc1c23 ; CC1(C)c2ccccc2-c2ccc(N(c3ccc4c(c3)-c3c(-c5ccccc5)ccc5cccc(c35)C4(C)C)c3cccc4ccccc34)cc21]

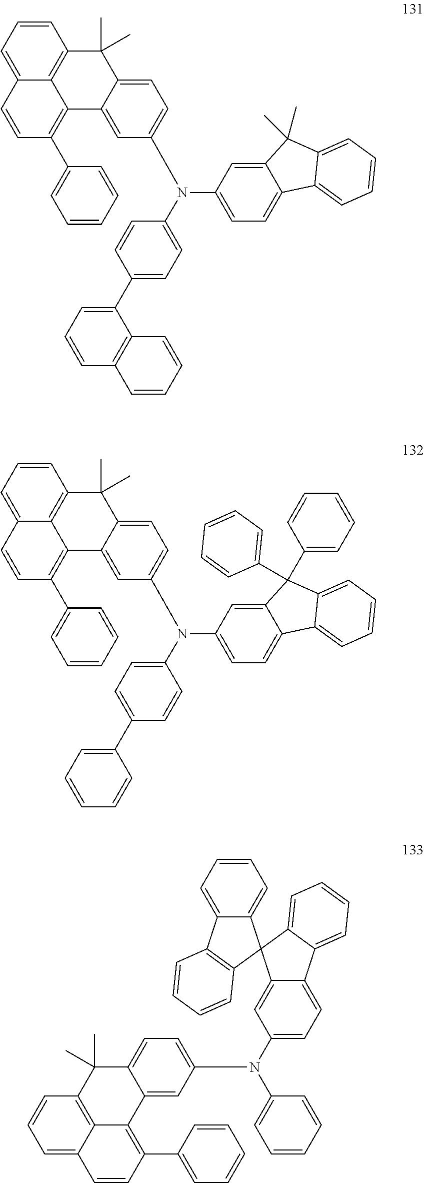 [SMILES: CC1(C)c2ccccc2-c2ccc(N(c3ccc(-c4cccc5ccccc45)cc3)c3ccc4c(c3)-c3c(-c5ccccc5)ccc5cccc(c35)C4(C)C)cc21 ; CC1(C)c2ccc(N(c3ccc(-c4ccccc4)cc3)c3ccc4c(c3)C(c3ccccc3)(c3ccccc3)c3ccccc3-4)cc2-c2c(-c3ccccc3)ccc3cccc1c23 ; CC1(C)c2ccc(N(c3ccccc3)c3ccc4c(c3)C3(c5ccccc5-c5ccccc53)c3ccccc3-4)cc2-c2c(-c3ccccc3)ccc3cccc1c23]

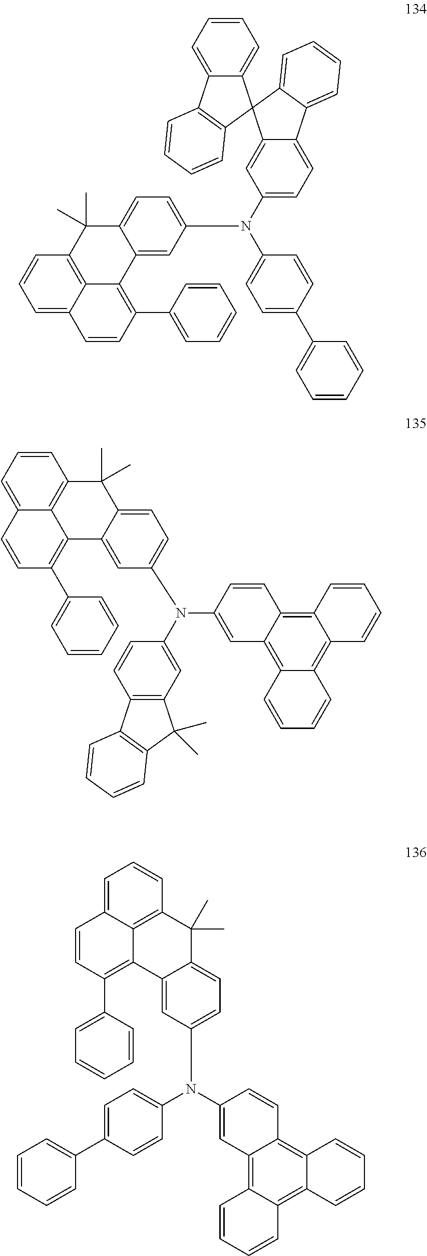 [SMILES: CC1(C)c2ccc(N(c3ccc(-c4ccccc4)cc3)c3ccc4c(c3)C3(c5ccccc5-c5ccccc53)c3ccccc3-4)cc2-c2c(-c3ccccc3)ccc3cccc1c23 ; CC1(C)c2ccccc2-c2ccc(N(c3ccc4c(c3)-c3c(-c5ccccc5)ccc5cccc(c35)C4(C)C)c3ccc4c5ccccc5c5ccccc5c4c3)cc21 ; CC1(C)c2ccc(N(c3ccc(-c4ccccc4)cc3)c3ccc4c5ccccc5c5ccccc5c4c3)cc2-c2c(-c3ccccc3)ccc3cccc1c23]

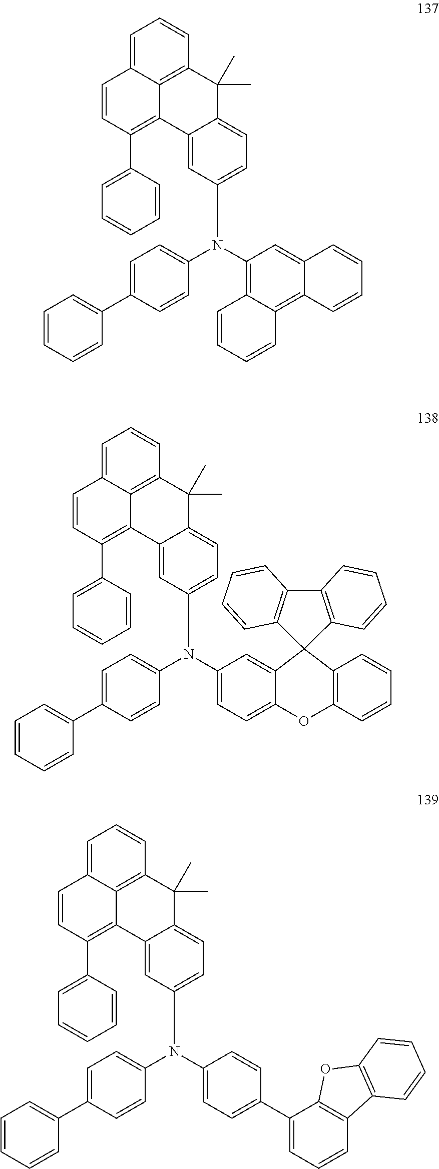 [SMILES: CC1(C)c2ccc(N(c3ccc(-c4ccccc4)cc3)c3cc4ccccc4c4ccccc34)cc2-c2c(-c3ccccc3)ccc3cccc1c23 ; CC1(C)c2ccc(N(c3ccc(-c4ccccc4)cc3)c3ccc4c(c3)C3(c5ccccc5O4)c4ccccc4-c4ccccc43)cc2-c2c(-c3ccccc3)ccc3cccc1c23 ; CC1(C)c2ccc(N(c3ccc(-c4ccccc4)cc3)c3ccc(-c4cccc5c4oc4ccccc45)cc3)cc2-c2c(-c3ccccc3)ccc3cccc1c23]

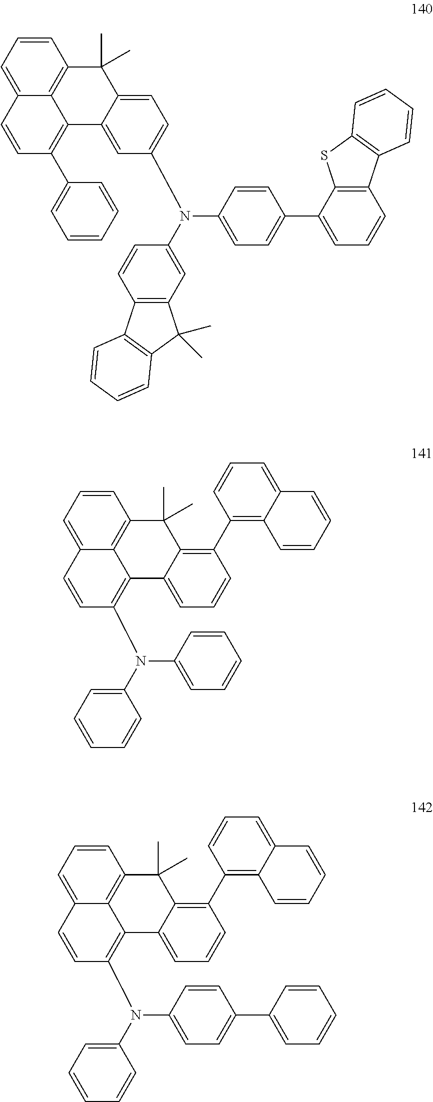 [SMILES: CC1(C)c2ccccc2-c2ccc(N(c3ccc(-c4cccc5c4sc4ccccc45)cc3)c3ccc4c(c3)-c3c(-c5ccccc5)ccc5cccc(c35)C4(C)C)cc21 ; CC1(C)c2c(-c3cccc4ccccc34)cccc2-c2c(N(c3ccccc3)c3ccccc3)ccc3cccc1c23 ; CC1(C)c2c(-c3cccc4ccccc34)cccc2-c2c(N(c3ccccc3)c3ccc(-c4ccccc4)cc3)ccc3cccc1c23]

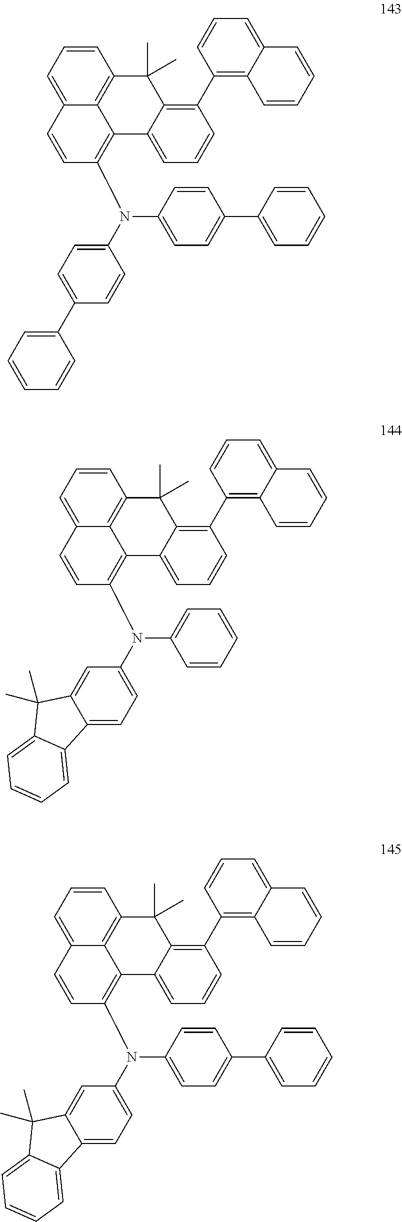 [SMILES: CC1(C)c2c(-c3cccc4ccccc34)cccc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3cccc(-c4cccc6ccccc46)c3C5(C)C)cc21 ; CC1(C)c2ccccc2-c2ccc(N(c3ccc(-c4ccccc4)cc3)c3ccc4cccc5c4c3-c3cccc(-c4cccc6ccccc46)c3C5(C)C)cc21]

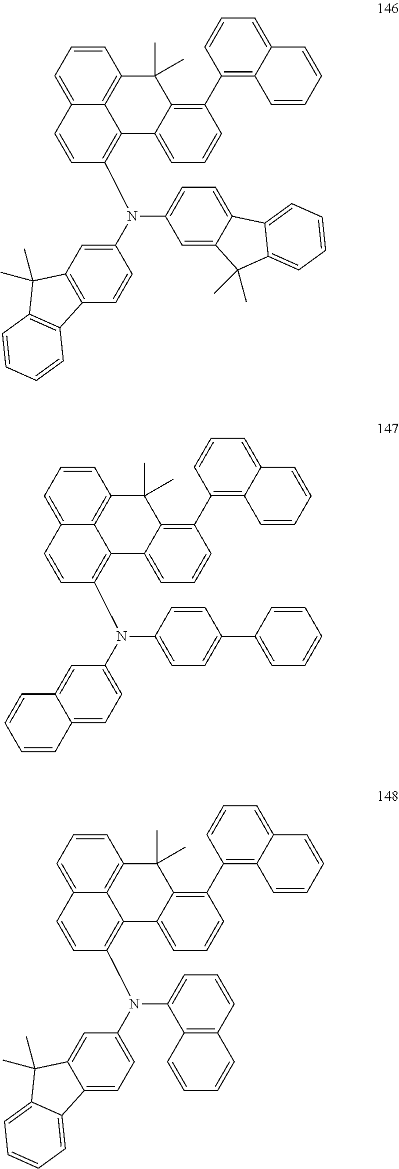 [SMILES: CC1(C)c2ccccc2-c2ccc(N(c3ccc4c(c3)C(C)(C)c3ccccc3-4)c3ccc4cccc5c4c3-c3cccc(-c4cccc6ccccc46)c3C5(C)C)cc21 ; CC1(C)c2c(-c3cccc4ccccc34)cccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4cccc6ccccc46)c3C5(C)C)c3cccc4ccccc34)cc21]

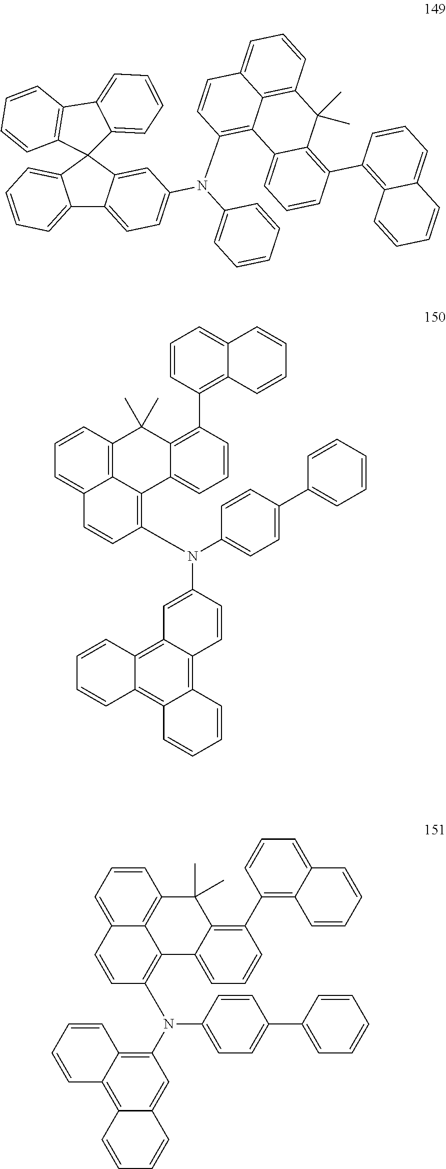 [SMILES: CC1(C)c2c(-c3cccc4ccccc34)cccc2-c2c(N(c3ccccc3)c3ccc4c(c3)C3(c5ccccc5-c5ccccc53)c3ccccc3-4)ccc3cccc1c23 ; CC1(C)c2c(-c3cccc4ccccc34)cccc2-c2c(N(c3ccc(-c4ccccc4)cc3)c3ccc4c5ccccc5c5ccccc5c4c3)ccc3cccc1c23 ; CC1(C)c2c(-c3cccc4ccccc34)cccc2-c2c(N(c3ccc(-c4ccccc4)cc3)c3cc4ccccc4c4ccccc34)ccc3cccc1c23]

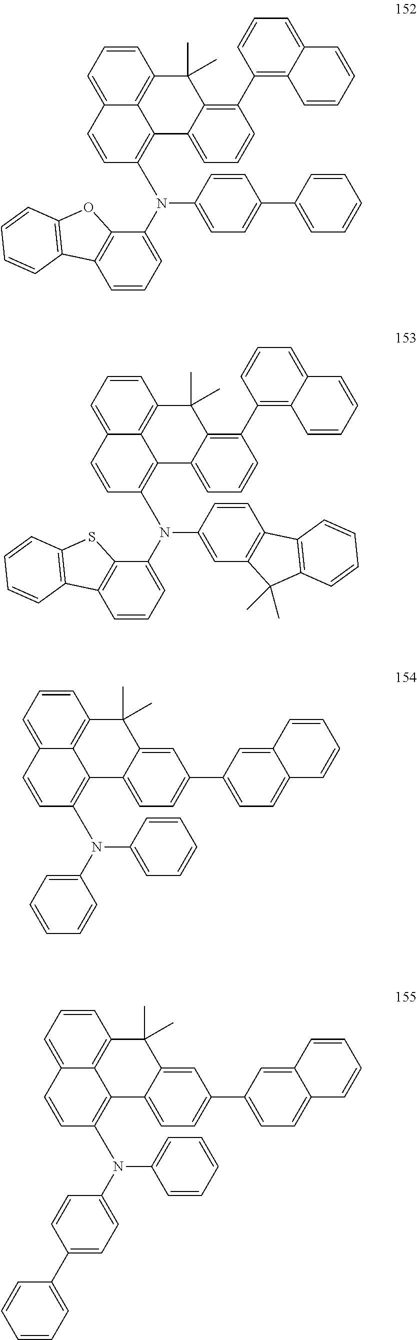 [SMILES: CC1(C)c2c(-c3cccc4ccccc34)cccc2-c2c(N(c3ccc(-c4ccccc4)cc3)c3cccc4c3oc3ccccc34)ccc3cccc1c23 ; CC1(C)c2ccccc2-c2ccc(N(c3ccc4cccc5c4c3-c3cccc(-c4cccc6ccccc46)c3C5(C)C)c3cccc4c3sc3ccccc34)cc21 ; CC1(C)c2cc(-c3ccc4ccccc4c3)ccc2-c2c(N(c3ccccc3)c3ccccc3)ccc3cccc1c23 ; CC1(C)c2cc(-c3ccc4ccccc4c3)ccc2-c2c(N(c3ccccc3)c3ccc(-c4ccccc4)cc3)ccc3cccc1c23]

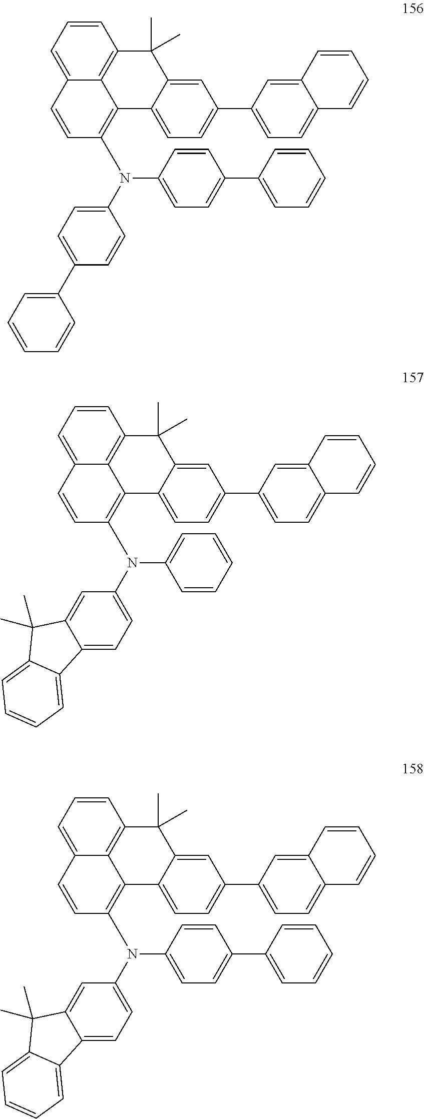 [SMILES: CC1(C)c2cc(-c3ccc4ccccc4c3)ccc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3ccc(-c4ccc6ccccc6c4)cc3C5(C)C)cc21 ; CC1(C)c2ccccc2-c2ccc(N(c3ccc(-c4ccccc4)cc3)c3ccc4cccc5c4c3-c3ccc(-c4ccc6ccccc6c4)cc3C5(C)C)cc21]

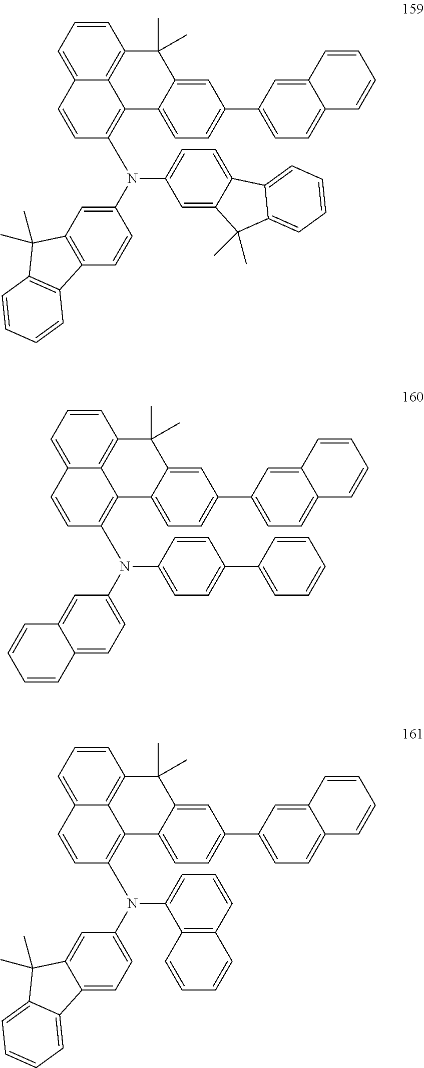 [SMILES: CC1(C)c2ccccc2-c2ccc(N(c3ccc4c(c3)C(C)(C)c3ccccc3-4)c3ccc4cccc5c4c3-c3ccc(-c4ccc6ccccc6c4)cc3C5(C)C)cc21 ; CC1(C)c2cc(-c3ccc4ccccc4c3)ccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccc(-c4ccc6ccccc6c4)cc3C5(C)C)c3cccc4ccccc34)cc21]

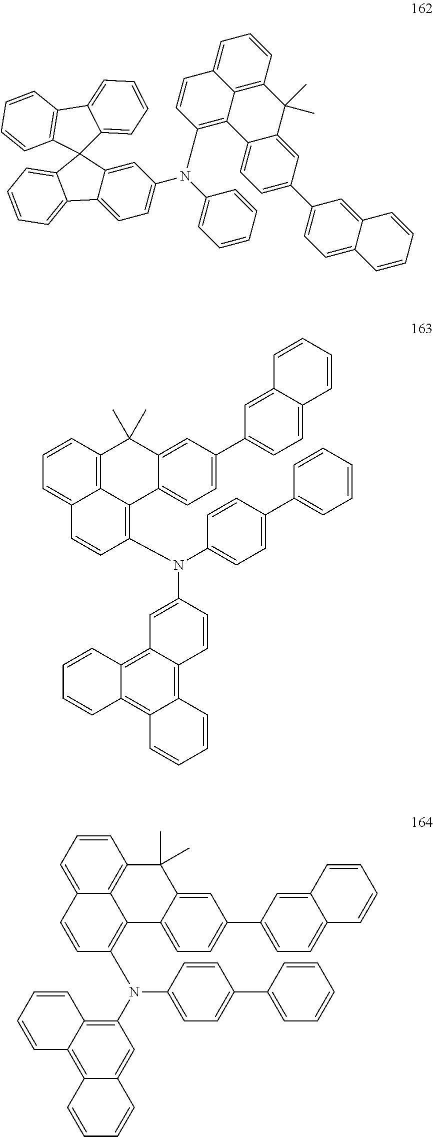 [SMILES: CC1(C)c2cc(-c3ccc4ccccc4c3)ccc2-c2c(N(c3ccccc3)c3ccc4c(c3)C3(c5ccccc5-c5ccccc53)c3ccccc3-4)ccc3cccc1c23 ; CC1(C)c2cc(-c3ccc4ccccc4c3)ccc2-c2c(N(c3ccc(-c4ccccc4)cc3)c3ccc4c5ccccc5c5ccccc5c4c3)ccc3cccc1c23 ; CC1(C)c2cc(-c3ccc4ccccc4c3)ccc2-c2c(N(c3ccc(-c4ccccc4)cc3)c3cc4ccccc4c4ccccc34)ccc3cccc1c23]

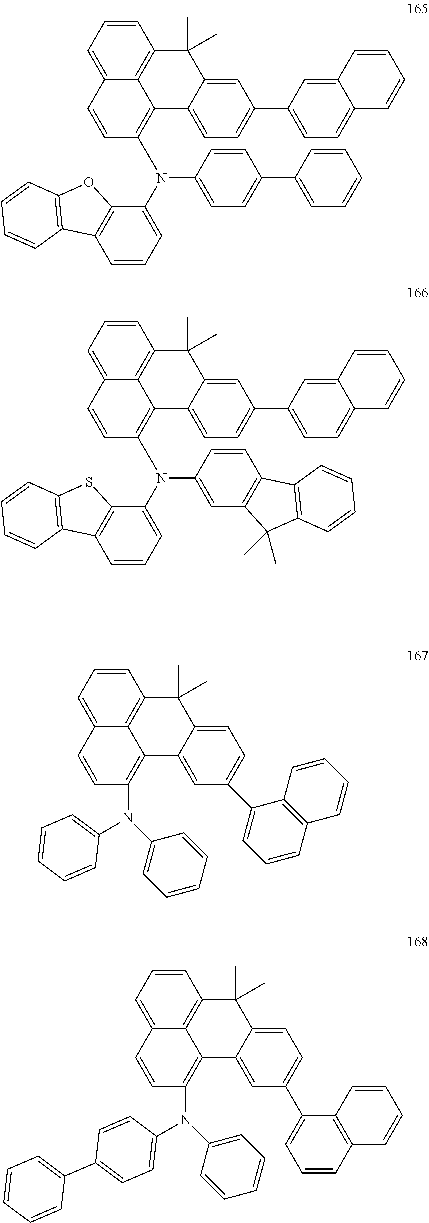 [SMILES: CC1(C)c2cc(-c3ccc4ccccc4c3)ccc2-c2c(N(c3ccc(-c4ccccc4)cc3)c3cccc4c3oc3ccccc34)ccc3cccc1c23 ; CC1(C)c2ccccc2-c2ccc(N(c3ccc4cccc5c4c3-c3ccc(-c4ccc6ccccc6c4)cc3C5(C)C)c3cccc4c3sc3ccccc34)cc21 ; CC1(C)c2ccc(-c3cccc4ccccc34)cc2-c2c(N(c3ccccc3)c3ccccc3)ccc3cccc1c23 ; CC1(C)c2ccc(-c3cccc4ccccc34)cc2-c2c(N(c3ccccc3)c3ccc(-c4ccccc4)cc3)ccc3cccc1c23]

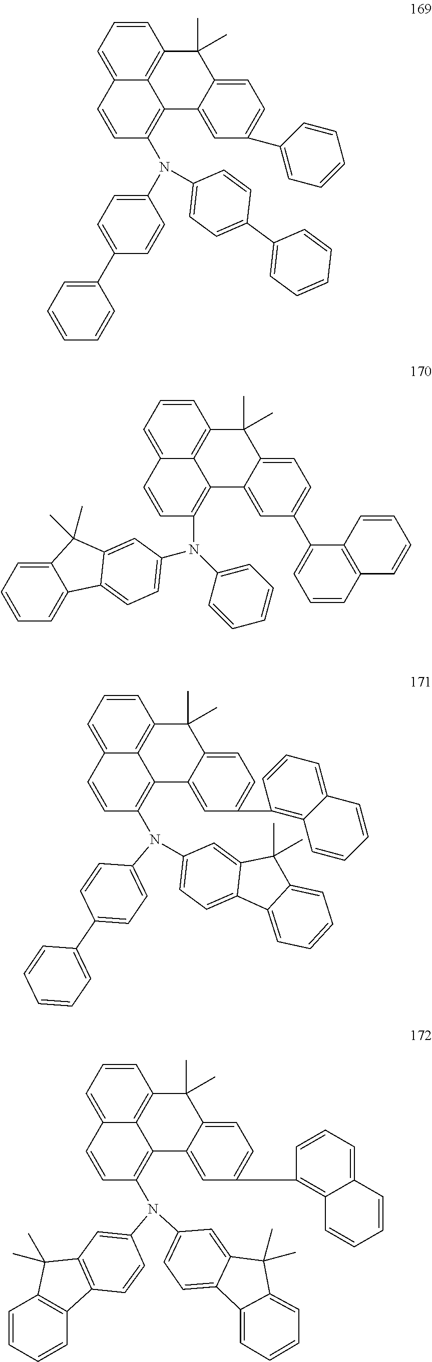 [SMILES: CC1(C)c2ccc(-c3ccccc3)cc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3cc(-c4cccc6ccccc46)ccc3C5(C)C)cc21 ; CC1(C)c2ccccc2-c2ccc(N(c3ccc(-c4ccccc4)cc3)c3ccc4cccc5c4c3-c3cc(-c4cccc6ccccc46)ccc3C5(C)C)cc21 ; CC1(C)c2ccccc2-c2ccc(N(c3ccc4c(c3)C(C)(C)c3ccccc3-4)c3ccc4cccc5c4c3-c3cc(-c4cccc6ccccc46)ccc3C5(C)C)cc21]

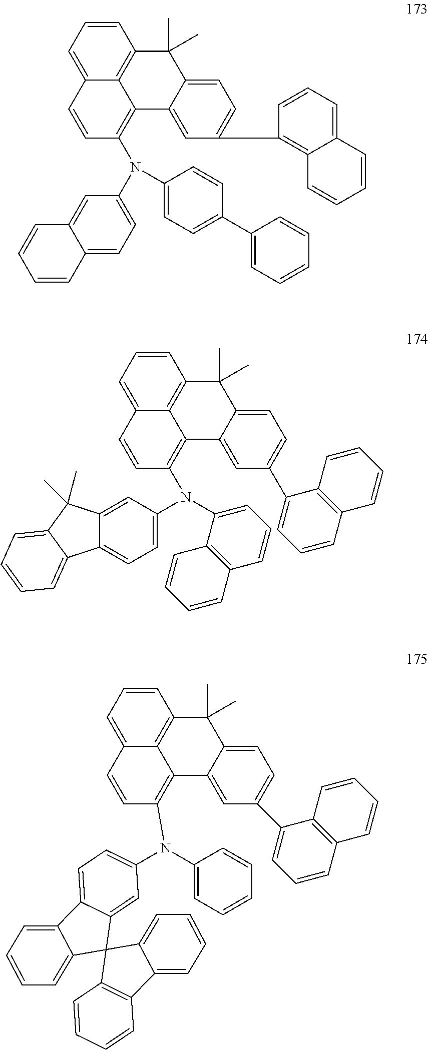 [SMILES: CC1(C)c2ccc(-c3cccc4ccccc34)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cc(-c4cccc6ccccc46)ccc3C5(C)C)c3cccc4ccccc34)cc21 ; CC1(C)c2ccc(-c3cccc4ccccc34)cc2-c2c(N(c3ccccc3)c3ccc4c(c3)C3(c5ccccc5-c5ccccc53)c3ccccc3-4)ccc3cccc1c23]

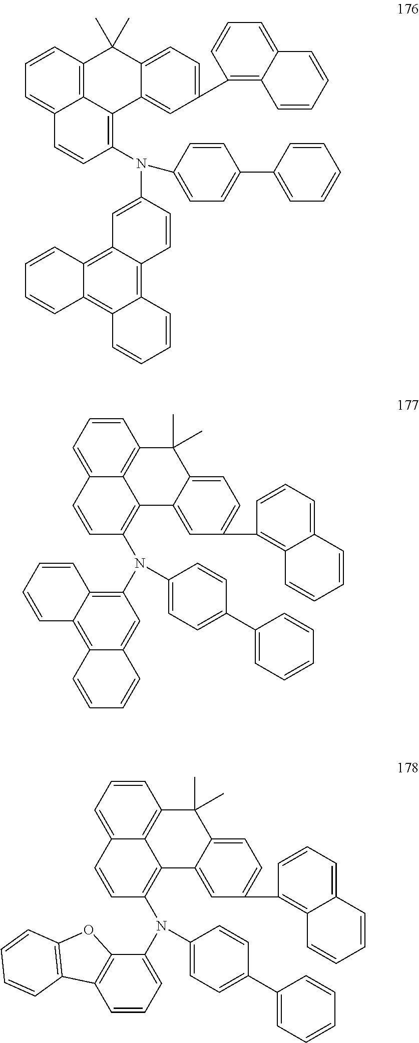 [SMILES: CC1(C)c2ccc(-c3cccc4ccccc34)cc2-c2c(N(c3ccc(-c4ccccc4)cc3)c3ccc4c5ccccc5c5ccccc5c4c3)ccc3cccc1c23 ; CC1(C)c2ccc(-c3cccc4ccccc34)cc2-c2c(N(c3ccc(-c4ccccc4)cc3)c3cc4ccccc4c4ccccc34)ccc3cccc1c23 ; CC1(C)c2ccc(-c3cccc4ccccc34)cc2-c2c(N(c3ccc(-c4ccccc4)cc3)c3cccc4c3oc3ccccc34)ccc3cccc1c23]

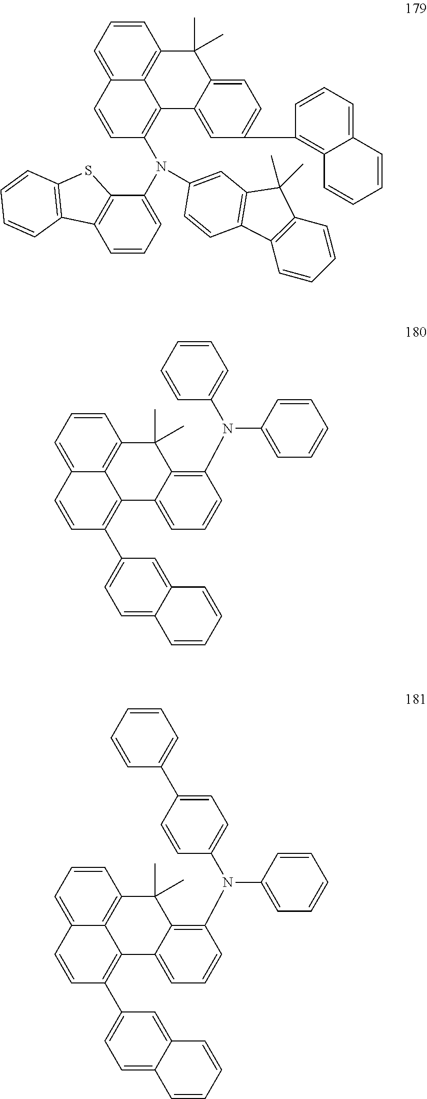 [SMILES: CC1(C)c2ccccc2-c2ccc(N(c3ccc4cccc5c4c3-c3cc(-c4cccc6ccccc46)ccc3C5(C)C)c3cccc4c3sc3ccccc34)cc21 ; CC1(C)c2c(cccc2N(c2ccccc2)c2ccccc2)-c2c(-c3ccc4ccccc4c3)ccc3cccc1c23 ; CC1(C)c2c(cccc2N(c2ccccc2)c2ccc(-c3ccccc3)cc2)-c2c(-c3ccc4ccccc4c3)ccc3cccc1c23]

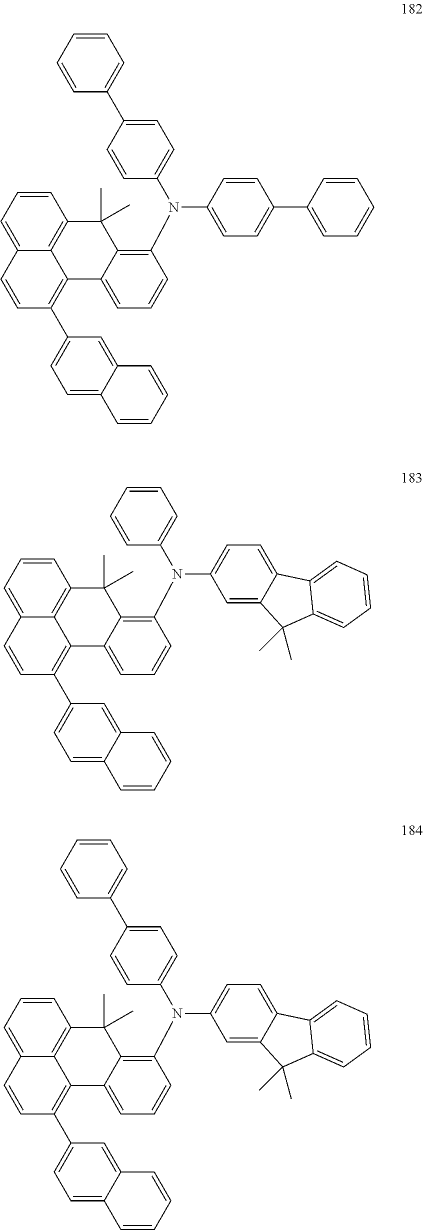 [SMILES: CC1(C)c2c(cccc2N(c2ccc(-c3ccccc3)cc2)c2ccc(-c3ccccc3)cc2)-c2c(-c3ccc4ccccc4c3)ccc3cccc1c23 ; CC1(C)c2ccccc2-c2ccc(N(c3ccccc3)c3cccc4c3C(C)(C)c3cccc5ccc(-c6ccc7ccccc7c6)c-4c35)cc21 ; CC1(C)c2ccccc2-c2ccc(N(c3ccc(-c4ccccc4)cc3)c3cccc4c3C(C)(C)c3cccc5ccc(-c6ccc7ccccc7c6)c-4c35)cc21]

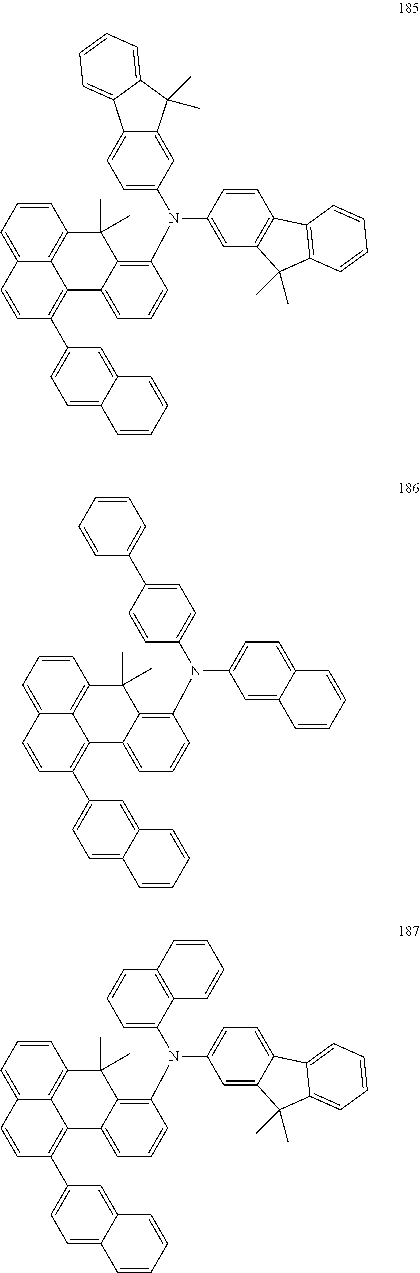 [SMILES: CC1(C)c2ccccc2-c2ccc(N(c3ccc4c(c3)C(C)(C)c3ccccc3-4)c3cccc4c3C(C)(C)c3cccc5ccc(-c6ccc7ccccc7c6)c-4c35)cc21 ; CC1(C)c2c(cccc2N(c2ccc(-c3ccccc3)cc2)c2ccc3ccccc3c2)-c2c(-c3ccc4ccccc4c3)ccc3cccc1c23 ; CC1(C)c2ccccc2-c2ccc(N(c3cccc4c3C(C)(C)c3cccc5ccc(-c6ccc7ccccc7c6)c-4c35)c3cccc4ccccc34)cc21]

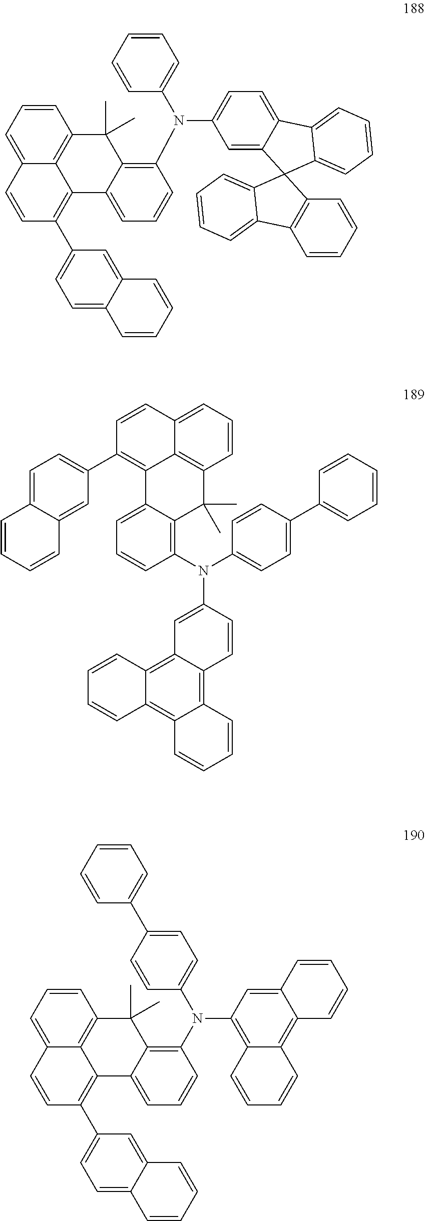 [SMILES: CC1(C)c2c(cccc2N(c2ccccc2)c2ccc3c(c2)C2(c4ccccc4-c4ccccc42)c2ccccc2-3)-c2c(-c3ccc4ccccc4c3)ccc3cccc1c23 ; CC1(C)c2c(cccc2N(c2ccc(-c3ccccc3)cc2)c2ccc3c4ccccc4c4ccccc4c3c2)-c2c(-c3ccc4ccccc4c3)ccc3cccc1c23 ; CC1(C)c2c(cccc2N(c2ccc(-c3ccccc3)cc2)c2cc3ccccc3c3ccccc23)-c2c(-c3ccc4ccccc4c3)ccc3cccc1c23]

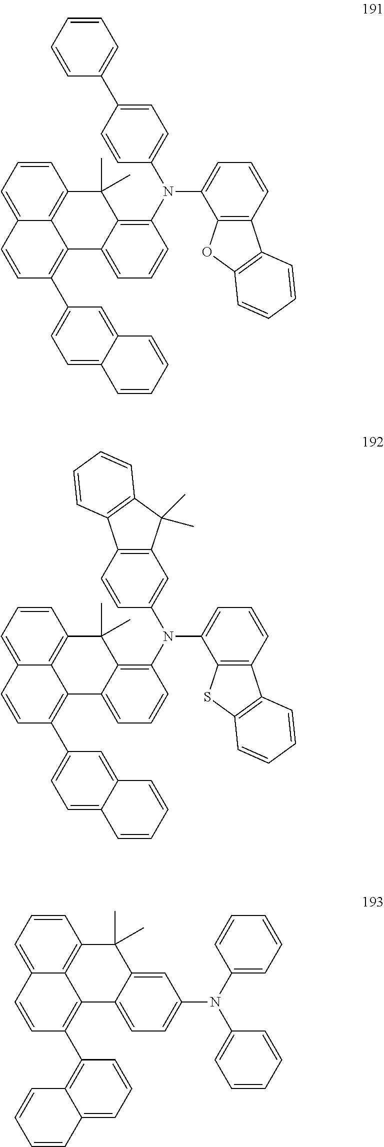 [SMILES: CC1(C)c2c(cccc2N(c2ccc(-c3ccccc3)cc2)c2cccc3c2oc2ccccc23)-c2c(-c3ccc4ccccc4c3)ccc3cccc1c23 ; CC1(C)c2ccccc2-c2ccc(N(c3cccc4c3C(C)(C)c3cccc5ccc(-c6ccc7ccccc7c6)c-4c35)c3cccc4c3sc3ccccc34)cc21 ; CC1(C)c2cc(N(c3ccccc3)c3ccccc3)ccc2-c2c(-c3cccc4ccccc34)ccc3cccc1c23]

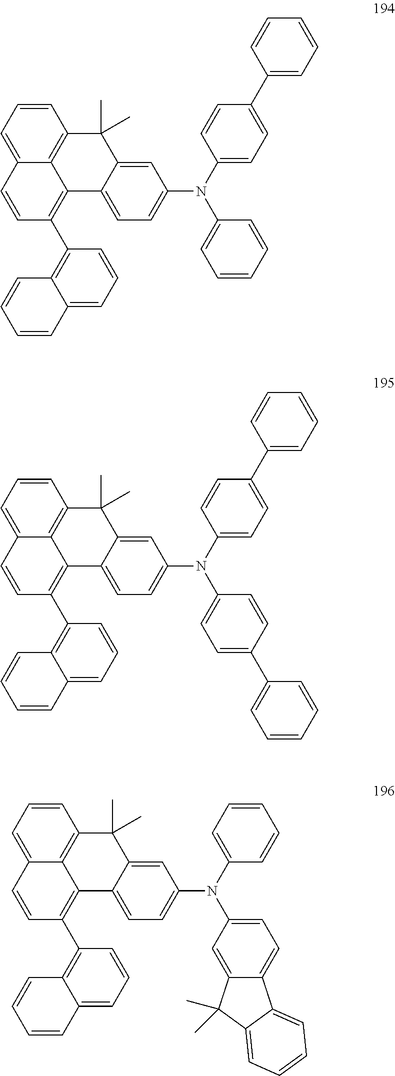 [SMILES: CC1(C)c2cc(N(c3ccccc3)c3ccc(-c4ccccc4)cc3)ccc2-c2c(-c3cccc4ccccc34)ccc3cccc1c23 ; CC1(C)c2cc(N(c3ccc(-c4ccccc4)cc3)c3ccc(-c4ccccc4)cc3)ccc2-c2c(-c3cccc4ccccc34)ccc3cccc1c23 ; CC1(C)c2ccccc2-c2ccc(N(c3ccccc3)c3ccc4c(c3)C(C)(C)c3cccc5ccc(-c6cccc7ccccc67)c-4c35)cc21]

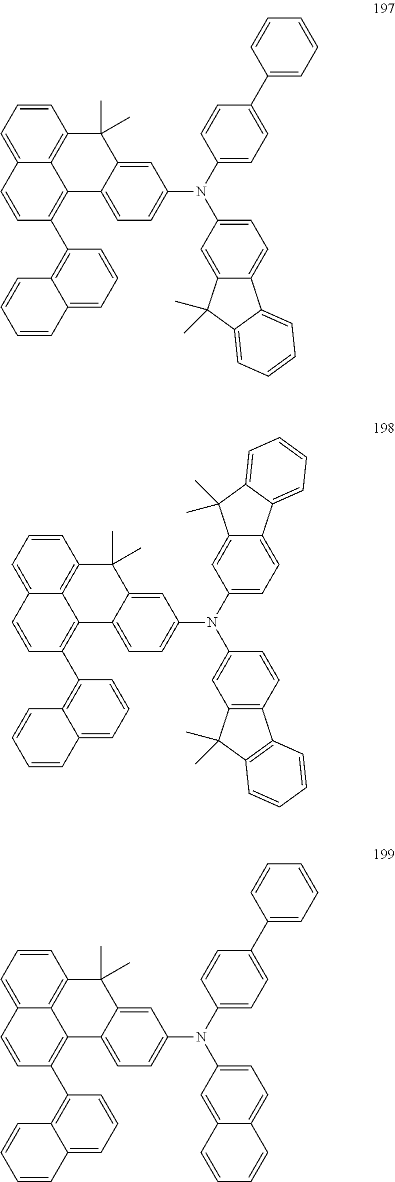 [SMILES: CC1(C)c2ccccc2-c2ccc(N(c3ccc(-c4ccccc4)cc3)c3ccc4c(c3)C(C)(C)c3cccc5ccc(-c6cccc7ccccc67)c-4c35)cc21 ; CC1(C)c2ccccc2-c2ccc(N(c3ccc4c(c3)C(C)(C)c3ccccc3-4)c3ccc4c(c3)C(C)(C)c3cccc5ccc(-c6cccc7ccccc67)c-4c35)cc21 ; CC1(C)c2cc(N(c3ccc(-c4ccccc4)cc3)c3ccc4ccccc4c3)ccc2-c2c(-c3cccc4ccccc34)ccc3cccc1c23]

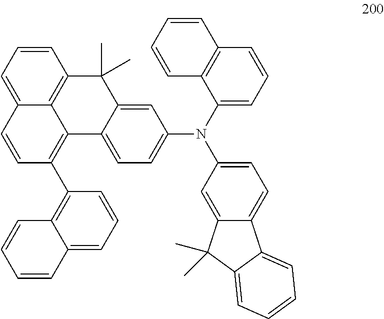 [SMILES: CC1(C)c2ccccc2-c2ccc(N(c3ccc4c(c3)C(C)(C)c3cccc5ccc(-c6cccc7ccccc67)c-4c35)c3cccc4ccccc34)cc21]

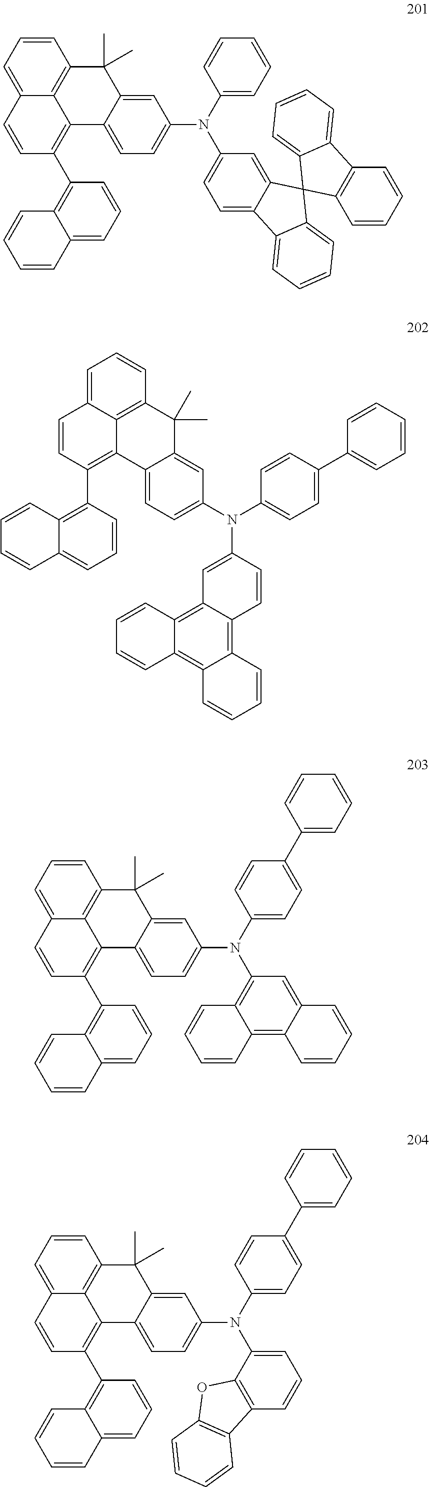 [SMILES: CC1(C)c2cc(N(c3ccccc3)c3ccc4c(c3)C3(c5ccccc5-c5ccccc53)c3ccccc3-4)ccc2-c2c(-c3cccc4ccccc34)ccc3cccc1c23 ; CC1(C)c2cc(N(c3ccc(-c4ccccc4)cc3)c3ccc4c5ccccc5c5ccccc5c4c3)ccc2-c2c(-c3cccc4ccccc34)ccc3cccc1c23 ; CC1(C)c2cc(N(c3ccc(-c4ccccc4)cc3)c3cc4ccccc4c4ccccc34)ccc2-c2c(-c3cccc4ccccc34)ccc3cccc1c23 ; CC1(C)c2cc(N(c3ccc(-c4ccccc4)cc3)c3cccc4c3oc3ccccc34)ccc2-c2c(-c3cccc4ccccc34)ccc3cccc1c23]

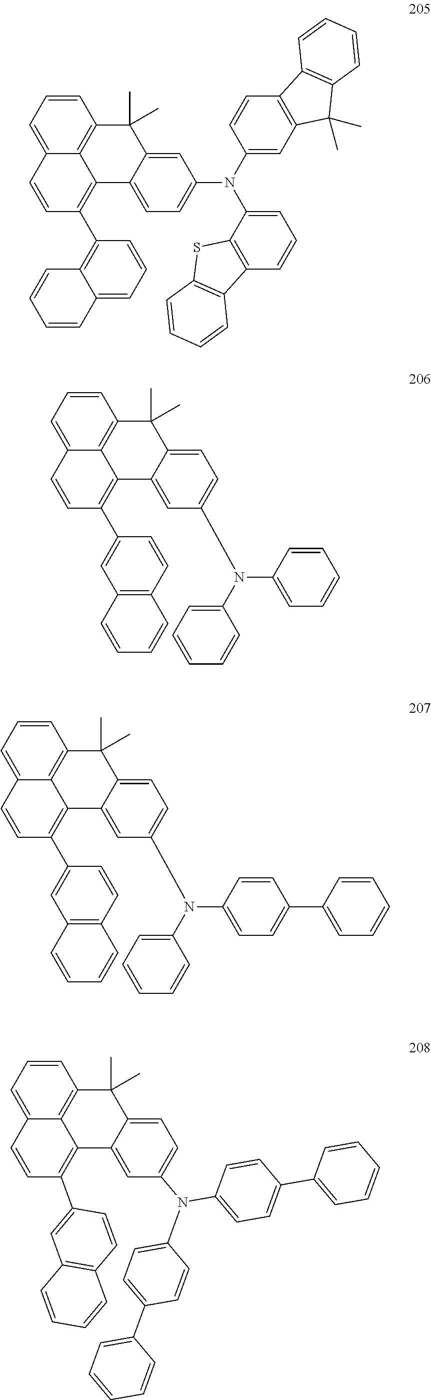 [SMILES: CC1(C)c2ccccc2-c2ccc(N(c3ccc4c(c3)C(C)(C)c3cccc5ccc(-c6cccc7ccccc67)c-4c35)c3cccc4c3sc3ccccc34)cc21 ; CC1(C)c2ccc(N(c3ccccc3)c3ccccc3)cc2-c2c(-c3ccc4ccccc4c3)ccc3cccc1c23 ; CC1(C)c2ccc(N(c3ccccc3)c3ccc(-c4ccccc4)cc3)cc2-c2c(-c3ccc4ccccc4c3)ccc3cccc1c23 ; CC1(C)c2ccc(N(c3ccc(-c4ccccc4)cc3)c3ccc(-c4ccccc4)cc3)cc2-c2c(-c3ccc4ccccc4c3)ccc3cccc1c23]

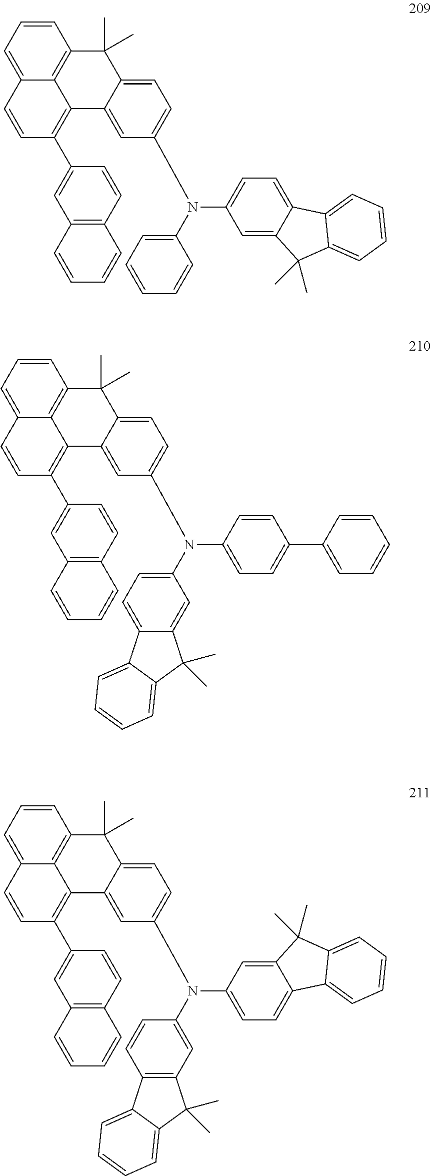 [SMILES: CC1(C)c2ccccc2-c2ccc(N(c3ccccc3)c3ccc4c(c3)-c3c(-c5ccc6ccccc6c5)ccc5cccc(c35)C4(C)C)cc21 ; CC1(C)c2ccccc2-c2ccc(N(c3ccc(-c4ccccc4)cc3)c3ccc4c(c3)-c3c(-c5ccc6ccccc6c5)ccc5cccc(c35)C4(C)C)cc21 ; CC1(C)c2ccccc2-c2ccc(N(c3ccc4c(c3)-c3c(-c5ccc6ccccc6c5)ccc5cccc(c35)C4(C)C)c3ccc4c(c3)C(C)(C)c3ccccc3-4)cc21]

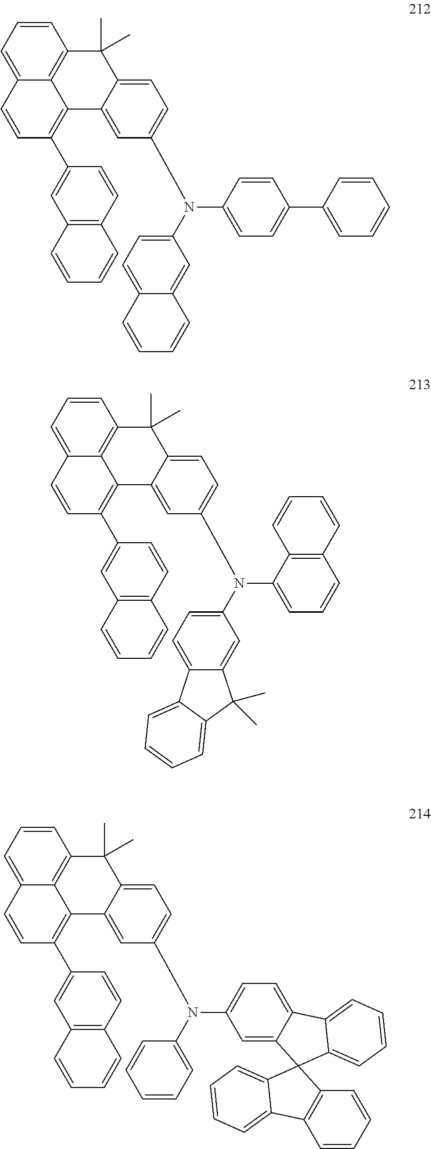

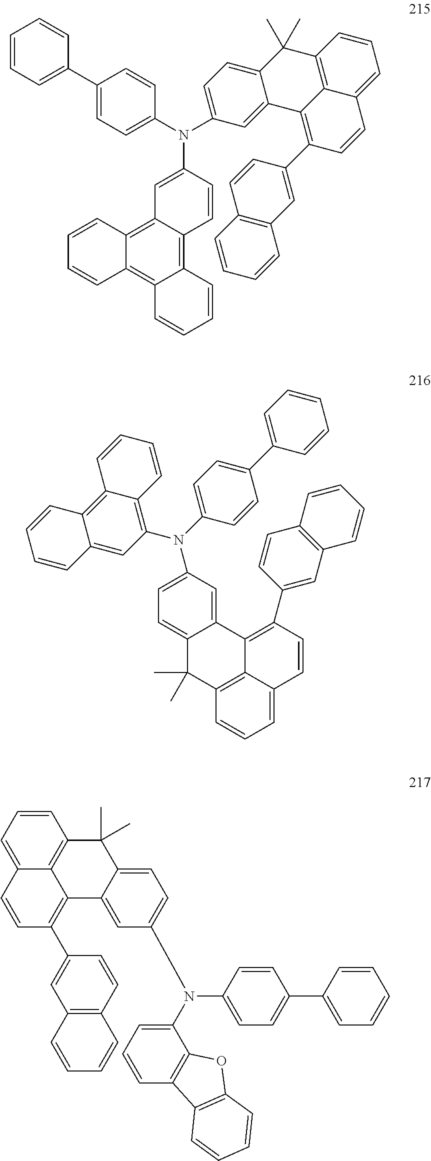 [SMILES: CC1(C)c2ccc(N(c3ccc(-c4ccccc4)cc3)c3ccc4c5ccccc5c5ccccc5c4c3)cc2-c2c(-c3ccc4ccccc4c3)ccc3cccc1c23 ; CC1(C)c2ccc(N(c3ccc(-c4ccccc4)cc3)c3cc4ccccc4c4ccccc34)cc2-c2c(-c3ccc4ccccc4c3)ccc3cccc1c23 ; CC1(C)c2ccc(N(c3ccc(-c4ccccc4)cc3)c3cccc4c3oc3ccccc34)cc2-c2c(-c3ccc4ccccc4c3)ccc3cccc1c23]

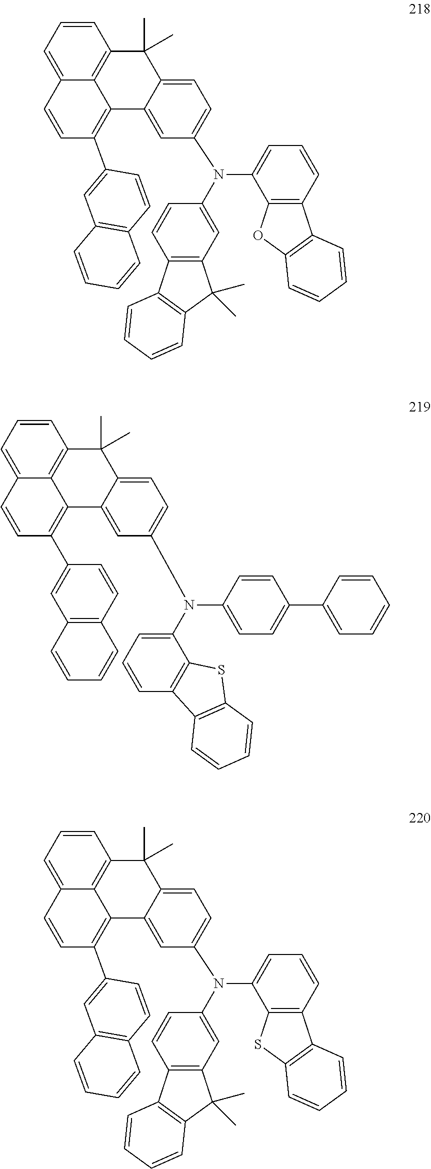 [SMILES: CC1(C)c2ccccc2-c2ccc(N(c3ccc4c(c3)-c3c(-c5ccc6ccccc6c5)ccc5cccc(c35)C4(C)C)c3cccc4c3oc3ccccc34)cc21 ; CC1(C)c2ccc(N(c3ccc(-c4ccccc4)cc3)c3cccc4c3sc3ccccc34)cc2-c2c(-c3ccc4ccccc4c3)ccc3cccc1c23 ; CC1(C)c2ccccc2-c2ccc(N(c3ccc4c(c3)-c3c(-c5ccc6ccccc6c5)ccc5cccc(c35)C4(C)C)c3cccc4c3sc3ccccc34)cc21]

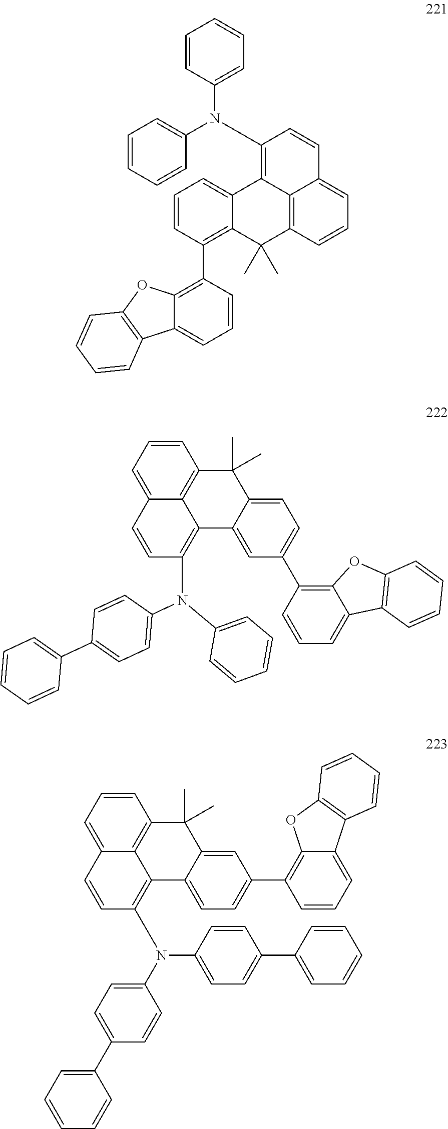 [SMILES: CC1(C)c2c(-c3cccc4c3oc3ccccc34)cccc2-c2c(N(c3ccccc3)c3ccccc3)ccc3cccc1c23 ; CC1(C)c2ccc(-c3cccc4c3oc3ccccc34)cc2-c2c(N(c3ccccc3)c3ccc(-c4ccccc4)cc3)ccc3cccc1c23 ; CC1(C)c2cc(-c3cccc4c3oc3ccccc34)ccc2-c2c(N(c3ccc(-c4ccccc4)cc3)c3ccc(-c4ccccc4)cc3)ccc3cccc1c23]

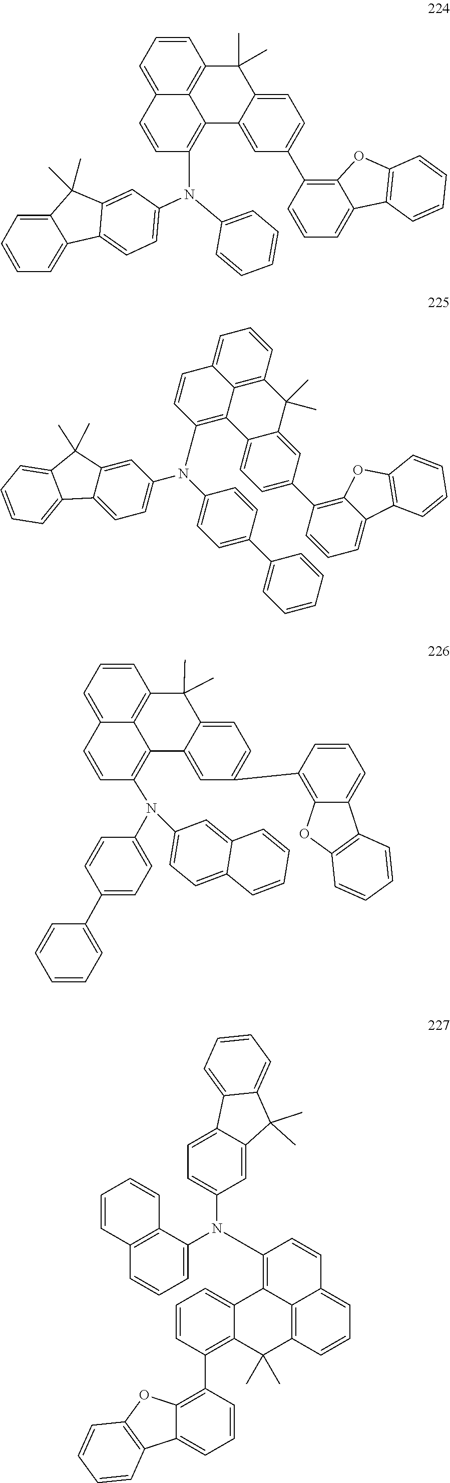 [SMILES: CC1(C)c2ccccc2-c2ccc(N(c3ccccc3)c3ccc4cccc5c4c3-c3cc(-c4cccc6c4oc4ccccc46)ccc3C5(C)C)cc21 ; CC1(C)c2ccccc2-c2ccc(N(c3ccc(-c4ccccc4)cc3)c3ccc4cccc5c4c3-c3ccc(-c4cccc6c4oc4ccccc46)cc3C5(C)C)cc21 ; CC1(C)c2ccc(-c3cccc4c3oc3ccccc34)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4cccc6c4oc4ccccc46)c3C5(C)C)c3cccc4ccccc34)cc21]

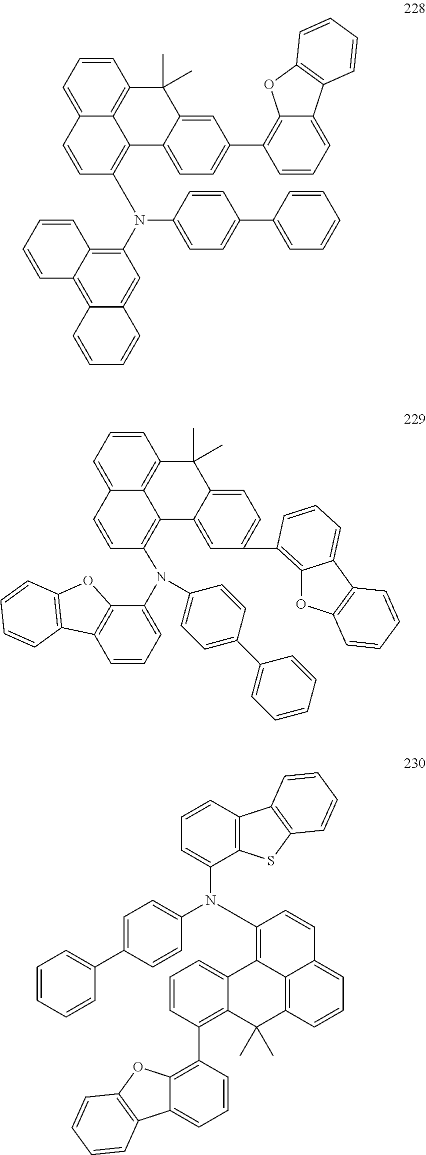 [SMILES: CC1(C)c2cc(-c3cccc4c3oc3ccccc34)ccc2-c2c(N(c3ccc(-c4ccccc4)cc3)c3cc4ccccc4c4ccccc34)ccc3cccc1c23 ; CC1(C)c2ccc(-c3cccc4c3oc3ccccc34)cc2-c2c(N(c3ccc(-c4ccccc4)cc3)c3cccc4c3oc3ccccc34)ccc3cccc1c23 ; CC1(C)c2c(-c3cccc4c3oc3ccccc34)cccc2-c2c(N(c3ccc(-c4ccccc4)cc3)c3cccc4c3sc3ccccc34)ccc3cccc1c23]

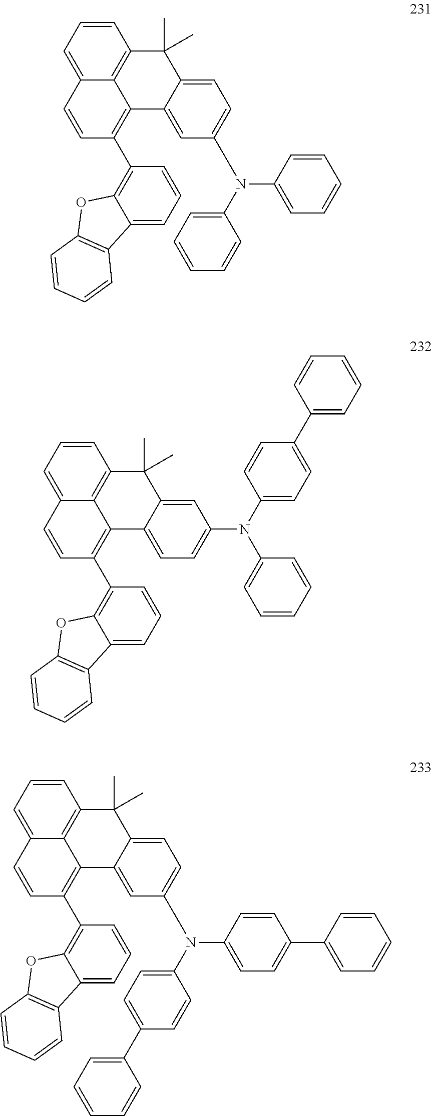 [SMILES: CC1(C)c2ccc(N(c3ccccc3)c3ccccc3)cc2-c2c(-c3cccc4c3oc3ccccc34)ccc3cccc1c23 ; CC1(C)c2cc(N(c3ccccc3)c3ccc(-c4ccccc4)cc3)ccc2-c2c(-c3cccc4c3oc3ccccc34)ccc3cccc1c23 ; CC1(C)c2ccc(N(c3ccc(-c4ccccc4)cc3)c3ccc(-c4ccccc4)cc3)cc2-c2c(-c3cccc4c3oc3ccccc34)ccc3cccc1c23]

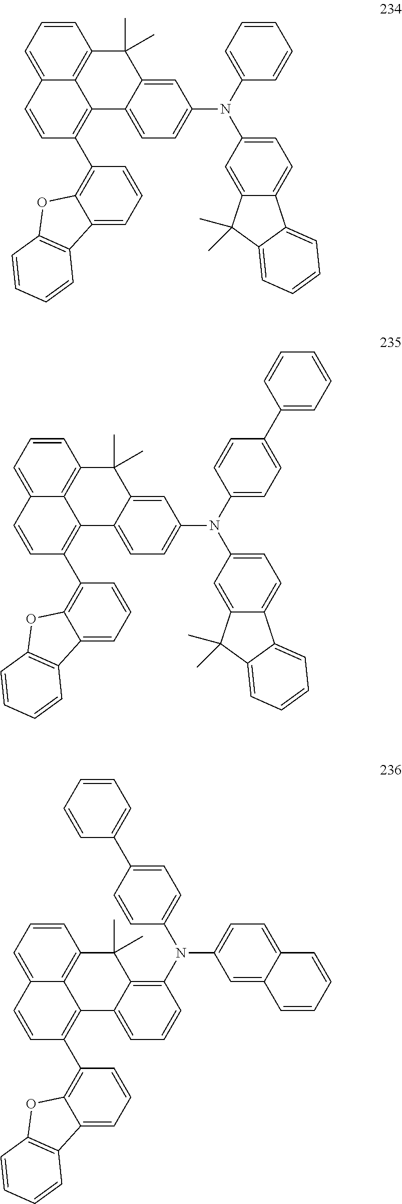 [SMILES: CC1(C)c2ccccc2-c2ccc(N(c3ccccc3)c3ccc4c(c3)C(C)(C)c3cccc5ccc(-c6cccc7c6oc6ccccc67)c-4c35)cc21 ; CC1(C)c2ccccc2-c2ccc(N(c3ccc(-c4ccccc4)cc3)c3ccc4c(c3)C(C)(C)c3cccc5ccc(-c6cccc7c6oc6ccccc67)c-4c35)cc21 ; CC1(C)c2c(cccc2N(c2ccc(-c3ccccc3)cc2)c2ccc3ccccc3c2)-c2c(-c3cccc4c3oc3ccccc34)ccc3cccc1c23]

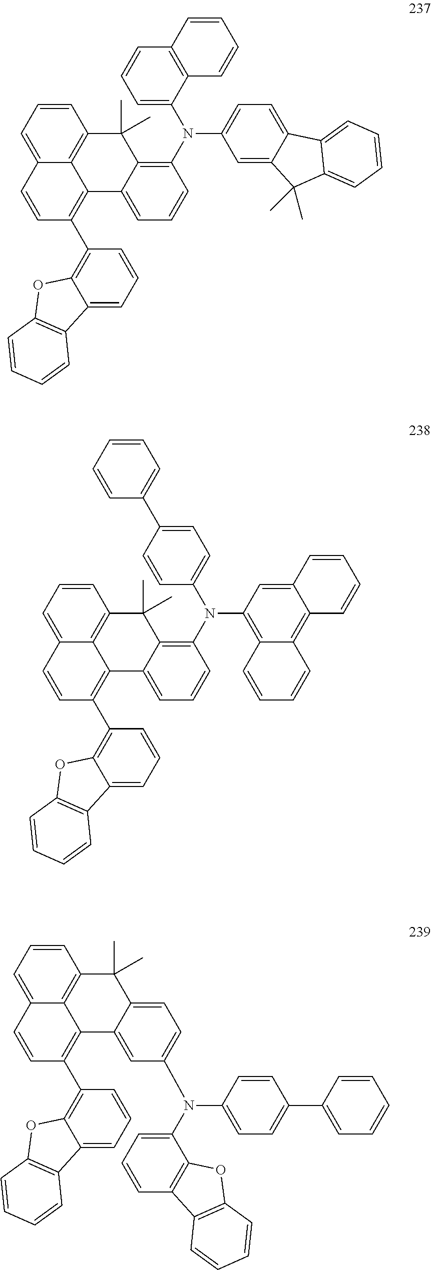 [SMILES: CC1(C)c2ccccc2-c2ccc(N(c3cccc4c3C(C)(C)c3cccc5ccc(-c6cccc7c6oc6ccccc67)c-4c35)c3cccc4ccccc34)cc21 ; CC1(C)c2c(cccc2N(c2ccc(-c3ccccc3)cc2)c2cc3ccccc3c3ccccc23)-c2c(-c3cccc4c3oc3ccccc34)ccc3cccc1c23 ; CC1(C)c2ccc(N(c3ccc(-c4ccccc4)cc3)c3cccc4c3oc3ccccc34)cc2-c2c(-c3cccc4c3oc3ccccc34)ccc3cccc1c23]

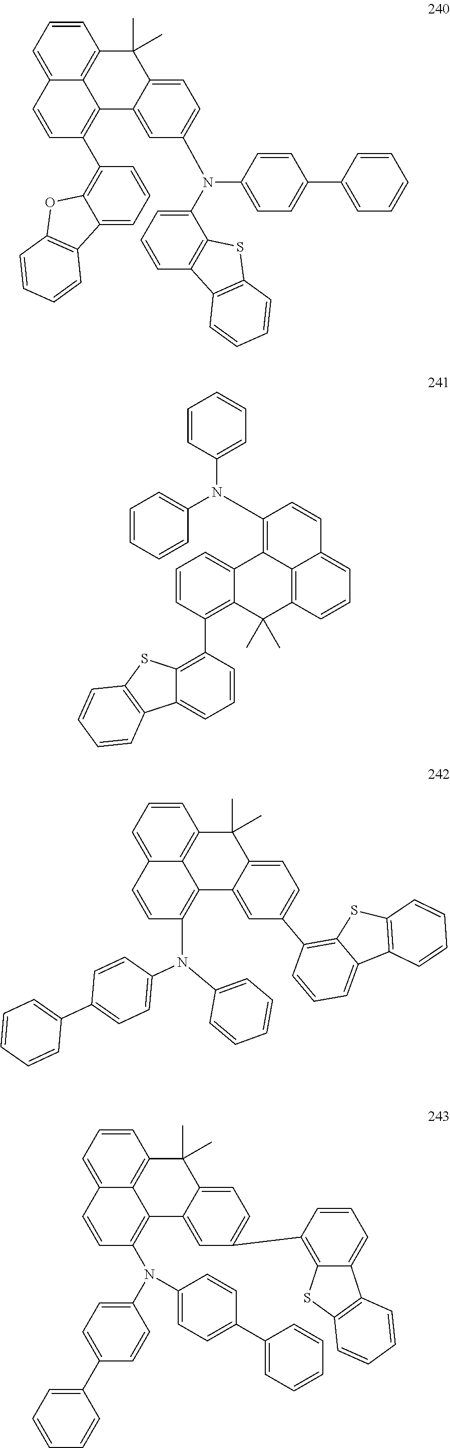 [SMILES: CC1(C)c2ccc(N(c3ccc(-c4ccccc4)cc3)c3cccc4c3sc3ccccc34)cc2-c2c(-c3cccc4c3oc3ccccc34)ccc3cccc1c23 ; CC1(C)c2c(-c3cccc4c3sc3ccccc34)cccc2-c2c(N(c3ccccc3)c3ccccc3)ccc3cccc1c23 ; CC1(C)c2ccc(-c3cccc4c3sc3ccccc34)cc2-c2c(N(c3ccccc3)c3ccc(-c4ccccc4)cc3)ccc3cccc1c23 ; CC1(C)c2ccc(-c3cccc4c3sc3ccccc34)cc2-c2c(N(c3ccc(-c4ccccc4)cc3)c3ccc(-c4ccccc4)cc3)ccc3cccc1c23]

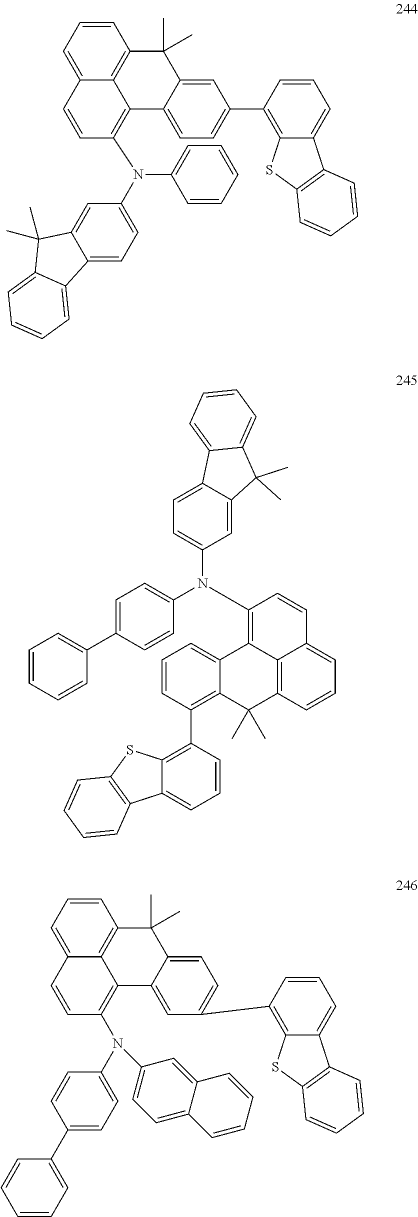 [SMILES: CC1(C)c2ccccc2-c2ccc(N(c3ccccc3)c3ccc4cccc5c4c3-c3ccc(-c4cccc6c4sc4ccccc46)cc3C5(C)C)cc21 ; CC1(C)c2ccccc2-c2ccc(N(c3ccc(-c4ccccc4)cc3)c3ccc4cccc5c4c3-c3cccc(-c4cccc6c4sc4ccccc46)c3C5(C)C)cc21 ; CC1(C)c2ccc(-c3cccc4c3sc3ccccc34)cc2-c2c(N(c3ccc(-c4ccccc4)cc3)c3ccc4ccccc4c3)ccc3cccc1c23]

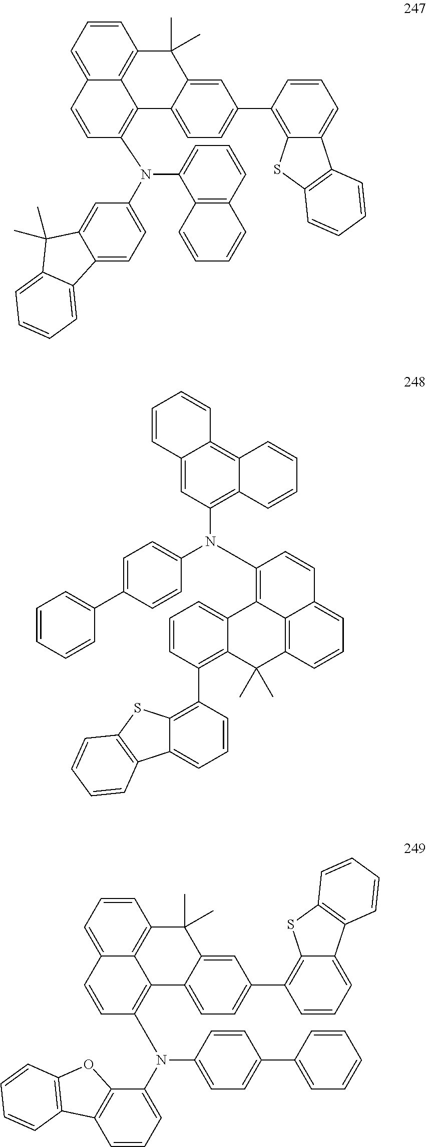 [SMILES: CC1(C)c2ccccc2-c2ccc(N(c3ccc4cccc5c4c3-c3ccc(-c4cccc6c4sc4ccccc46)cc3C5(C)C)c3cccc4ccccc34)cc21 ; CC1(C)c2c(-c3cccc4c3sc3ccccc34)cccc2-c2c(N(c3ccc(-c4ccccc4)cc3)c3cc4ccccc4c4ccccc34)ccc3cccc1c23 ; CC1(C)c2cc(-c3cccc4c3sc3ccccc34)ccc2-c2c(N(c3ccc(-c4ccccc4)cc3)c3cccc4c3oc3ccccc34)ccc3cccc1c23]

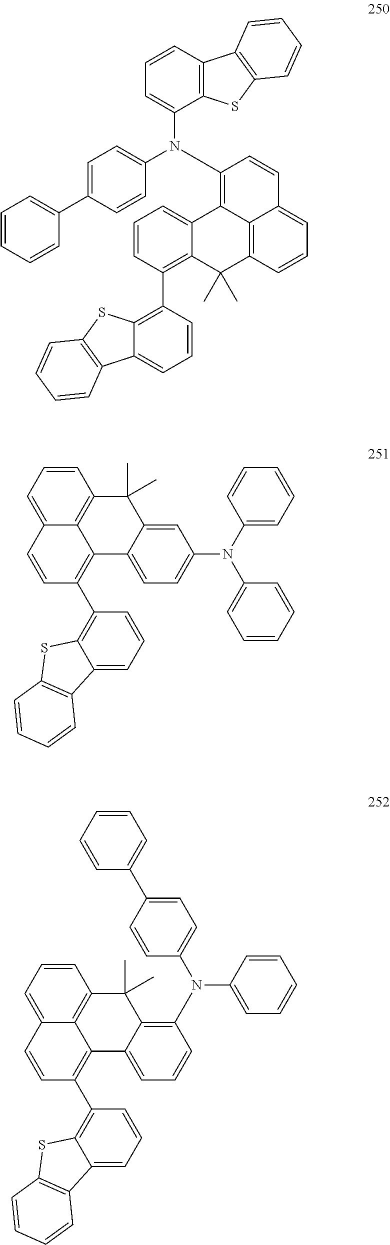 [SMILES: CC1(C)c2c(-c3cccc4c3sc3ccccc34)cccc2-c2c(N(c3ccc(-c4ccccc4)cc3)c3cccc4c3sc3ccccc34)ccc3cccc1c23 ; CC1(C)c2cc(N(c3ccccc3)c3ccccc3)ccc2-c2c(-c3cccc4c3sc3ccccc34)ccc3cccc1c23 ; CC1(C)c2c(cccc2N(c2ccccc2)c2ccc(-c3ccccc3)cc2)-c2c(-c3cccc4c3sc3ccccc34)ccc3cccc1c23]

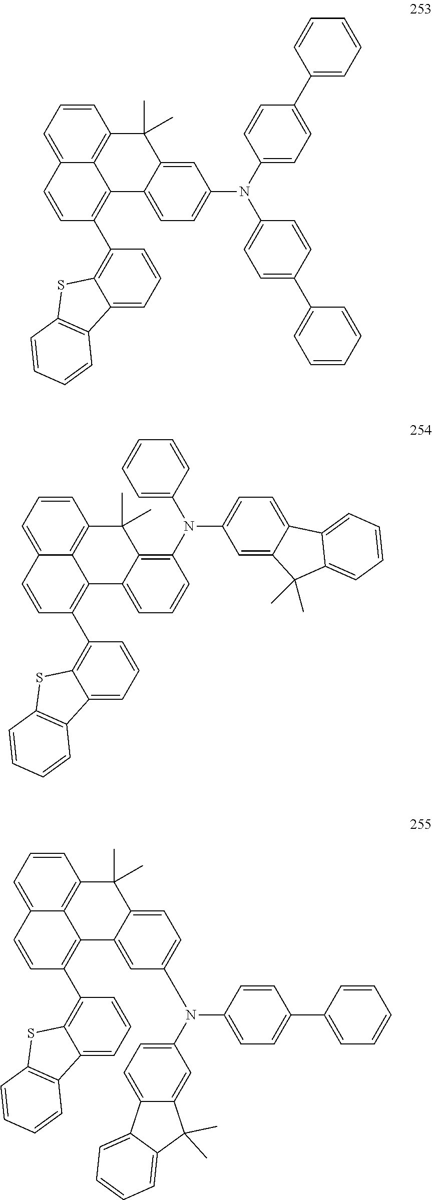 [SMILES: CC1(C)c2cc(N(c3ccc(-c4ccccc4)cc3)c3ccc(-c4ccccc4)cc3)ccc2-c2c(-c3cccc4c3sc3ccccc34)ccc3cccc1c23 ; CC1(C)c2ccccc2-c2ccc(N(c3ccccc3)c3cccc4c3C(C)(C)c3cccc5ccc(-c6cccc7c6sc6ccccc67)c-4c35)cc21 ; CC1(C)c2ccccc2-c2ccc(N(c3ccc(-c4ccccc4)cc3)c3ccc4c(c3)-c3c(-c5cccc6c5sc5ccccc56)ccc5cccc(c35)C4(C)C)cc21]

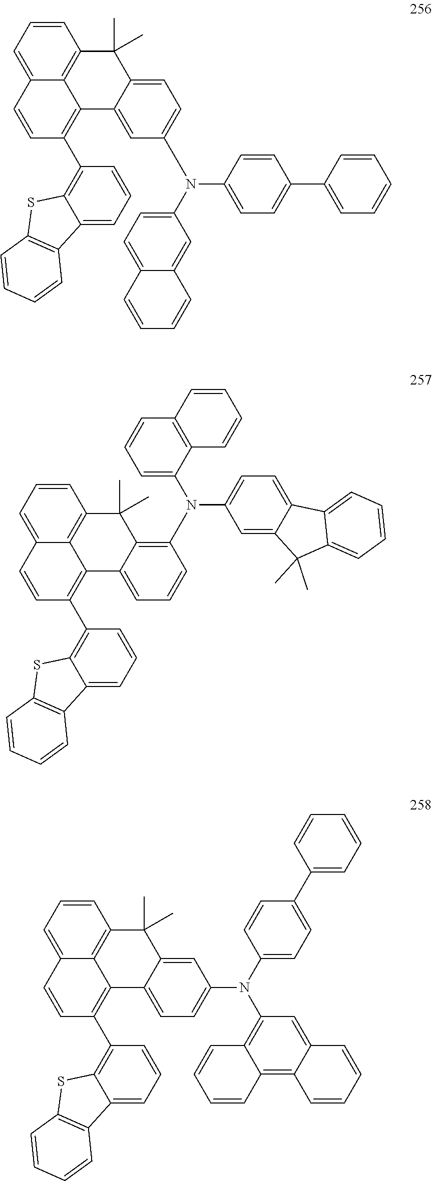 [SMILES: CC1(C)c2ccc(N(c3ccc(-c4ccccc4)cc3)c3ccc4ccccc4c3)cc2-c2c(-c3cccc4c3sc3ccccc34)ccc3cccc1c23 ; CC1(C)c2ccccc2-c2ccc(N(c3cccc4c3C(C)(C)c3cccc5ccc(-c6cccc7c6sc6ccccc67)c-4c35)c3cccc4ccccc34)cc21 ; CC1(C)c2cc(N(c3ccc(-c4ccccc4)cc3)c3cc4ccccc4c4ccccc34)ccc2-c2c(-c3cccc4c3sc3ccccc34)ccc3cccc1c23]

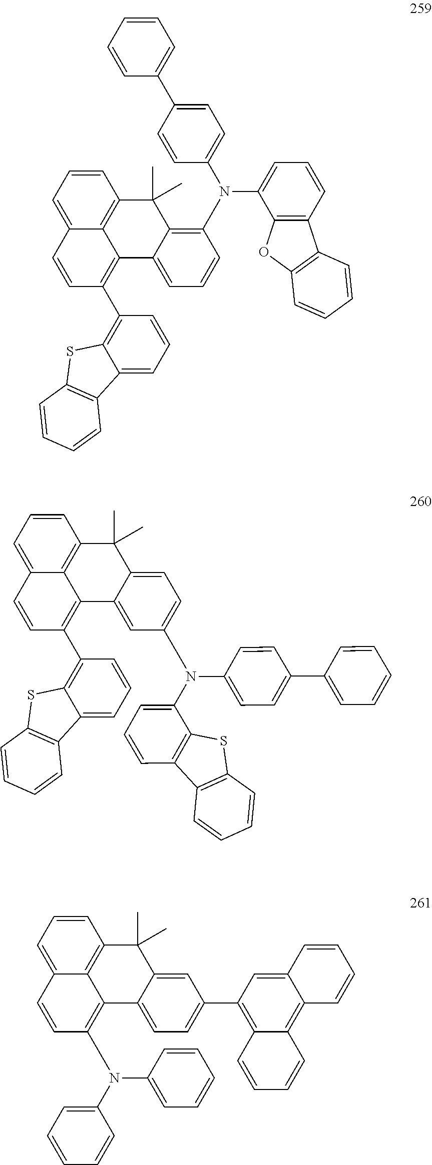 [SMILES: CC1(C)c2c(cccc2N(c2ccc(-c3ccccc3)cc2)c2cccc3c2oc2ccccc23)-c2c(-c3cccc4c3sc3ccccc34)ccc3cccc1c23 ; CC1(C)c2ccc(N(c3ccc(-c4ccccc4)cc3)c3cccc4c3sc3ccccc34)cc2-c2c(-c3cccc4c3sc3ccccc34)ccc3cccc1c23 ; CC1(C)c2cc(-c3cc4ccccc4c4ccccc34)ccc2-c2c(N(c3ccccc3)c3ccccc3)ccc3cccc1c23]

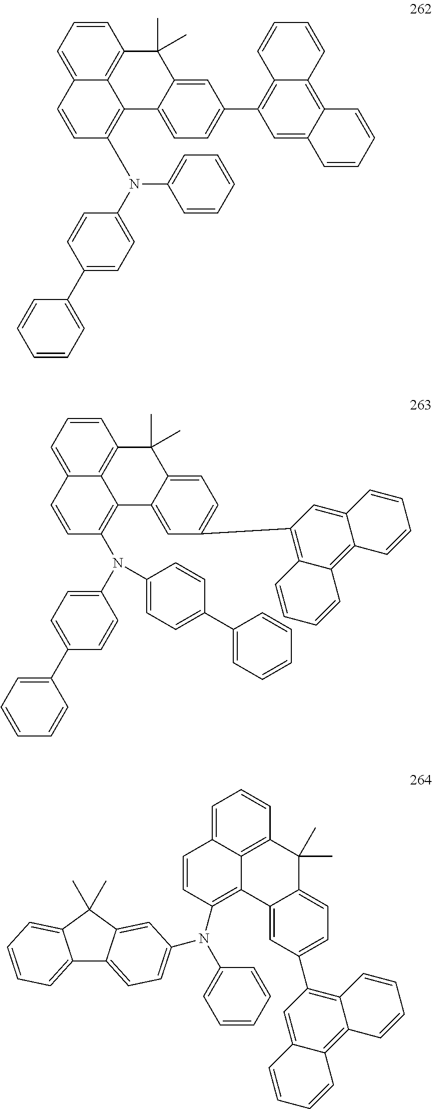 [SMILES: CC1(C)c2cc(-c3cc4ccccc4c4ccccc34)ccc2-c2c(N(c3ccccc3)c3ccc(-c4ccccc4)cc3)ccc3cccc1c23 ; CC1(C)c2ccc(-c3cc4ccccc4c4ccccc34)cc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3cc(-c4cc6ccccc6c6ccccc46)ccc3C5(C)C)cc21]

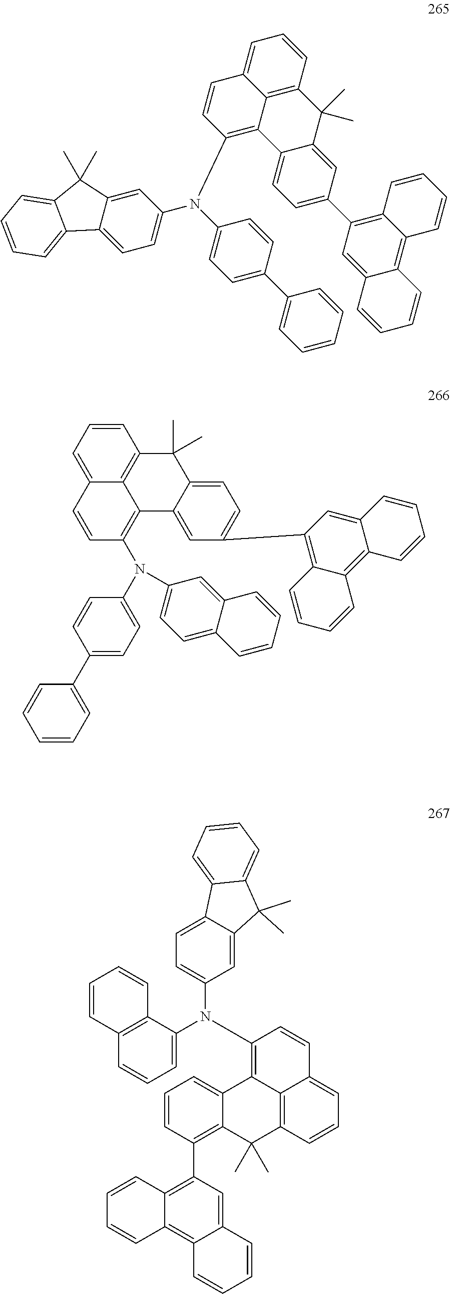 [SMILES: CC1(C)c2ccccc2-c2ccc(N(c3ccc(-c4ccccc4)cc3)c3ccc4cccc5c4c3-c3ccc(-c4cc6ccccc6c6ccccc46)cc3C5(C)C)cc21 ; CC1(C)c2ccc(-c3cc4ccccc4c4ccccc34)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4cc6ccccc6c6ccccc46)c3C5(C)C)c3cccc4ccccc34)cc21]

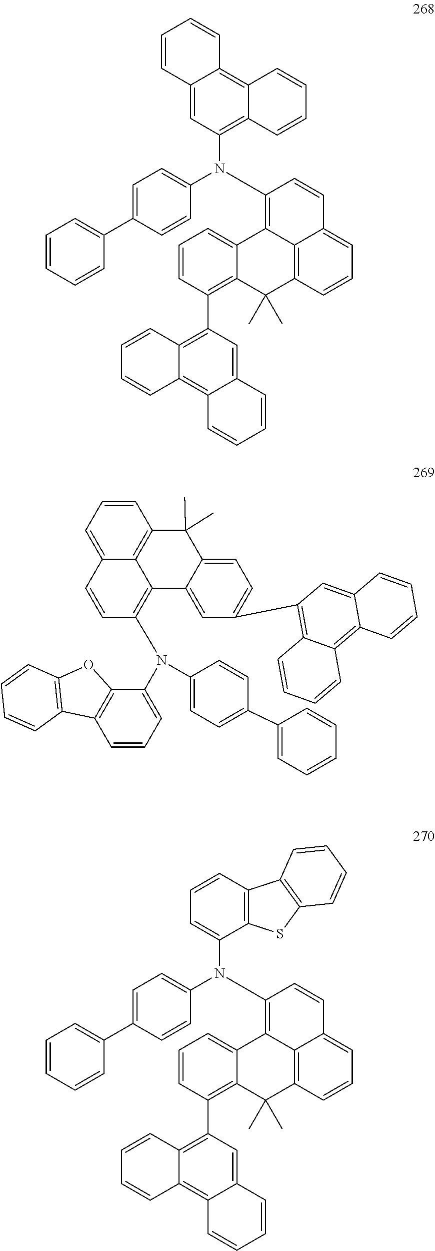 [SMILES: CC1(C)c2c(-c3cc4ccccc4c4ccccc34)cccc2-c2c(N(c3ccc(-c4ccccc4)cc3)c3cc4ccccc4c4ccccc34)ccc3cccc1c23 ; CC1(C)c2ccc(-c3cc4ccccc4c4ccccc34)cc2-c2c(N(c3ccc(-c4ccccc4)cc3)c3cccc4c3oc3ccccc34)ccc3cccc1c23 ; CC1(C)c2c(-c3cc4ccccc4c4ccccc34)cccc2-c2c(N(c3ccc(-c4ccccc4)cc3)c3cccc4c3sc3ccccc34)ccc3cccc1c23]

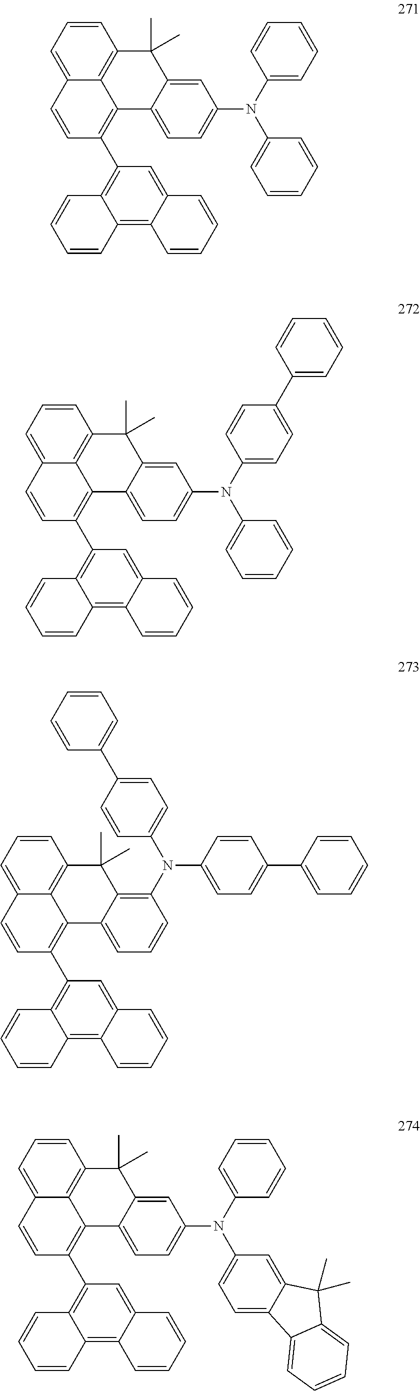 [SMILES: CC1(C)c2cc(N(c3ccccc3)c3ccccc3)ccc2-c2c(-c3cc4ccccc4c4ccccc34)ccc3cccc1c23 ; CC1(C)c2cc(N(c3ccccc3)c3ccc(-c4ccccc4)cc3)ccc2-c2c(-c3cc4ccccc4c4ccccc34)ccc3cccc1c23 ; CC1(C)c2c(cccc2N(c2ccc(-c3ccccc3)cc2)c2ccc(-c3ccccc3)cc2)-c2c(-c3cc4ccccc4c4ccccc34)ccc3cccc1c23 ; CC1(C)c2ccccc2-c2ccc(N(c3ccccc3)c3ccc4c(c3)C(C)(C)c3cccc5ccc(-c6cc7ccccc7c7ccccc67)c-4c35)cc21]

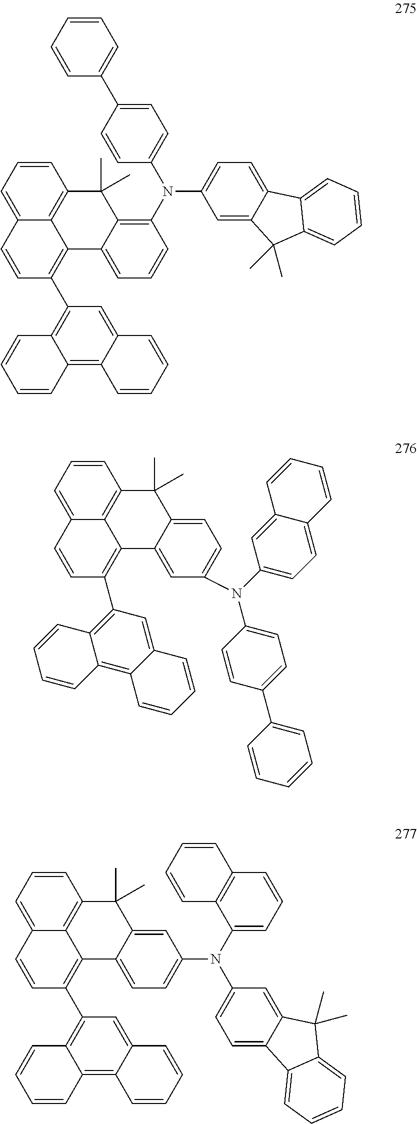 [SMILES: CC1(C)c2ccccc2-c2ccc(N(c3ccc(-c4ccccc4)cc3)c3cccc4c3C(C)(C)c3cccc5ccc(-c6cc7ccccc7c7ccccc67)c-4c35)cc21 ; CC1(C)c2ccc(N(c3ccc(-c4ccccc4)cc3)c3ccc4ccccc4c3)cc2-c2c(-c3cc4ccccc4c4ccccc34)ccc3cccc1c23 ; CC1(C)c2ccccc2-c2ccc(N(c3ccc4c(c3)C(C)(C)c3cccc5ccc(-c6cc7ccccc7c7ccccc67)c-4c35)c3cccc4ccccc34)cc21]

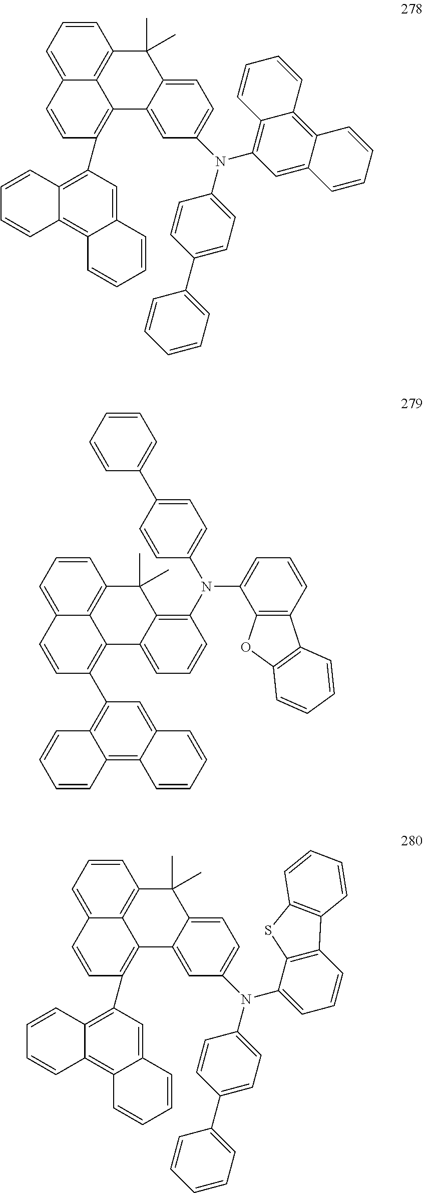 [SMILES: CC1(C)c2ccc(N(c3ccc(-c4ccccc4)cc3)c3cc4ccccc4c4ccccc34)cc2-c2c(-c3cc4ccccc4c4ccccc34)ccc3cccc1c23 ; CC1(C)c2c(cccc2N(c2ccc(-c3ccccc3)cc2)c2cccc3c2oc2ccccc23)-c2c(-c3cc4ccccc4c4ccccc34)ccc3cccc1c23 ; CC1(C)c2ccc(N(c3ccc(-c4ccccc4)cc3)c3cccc4c3sc3ccccc34)cc2-c2c(-c3cc4ccccc4c4ccccc34)ccc3cccc1c23]

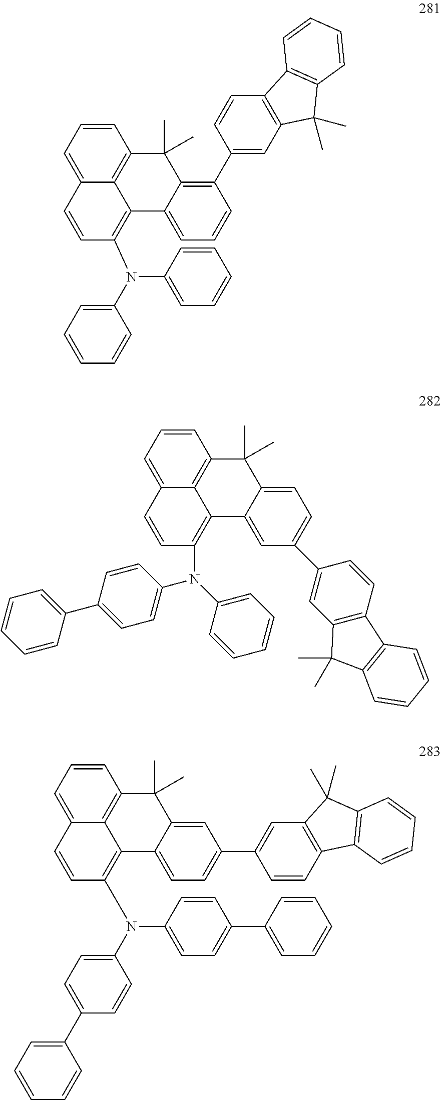 [SMILES: CC1(C)c2ccccc2-c2ccc(-c3cccc4c3C(C)(C)c3cccc5ccc(N(c6ccccc6)c6ccccc6)c-4c35)cc21 ; CC1(C)c2ccccc2-c2ccc(-c3ccc4c(c3)-c3c(N(c5ccccc5)c5ccc(-c6ccccc6)cc5)ccc5cccc(c35)C4(C)C)cc21 ; CC1(C)c2ccccc2-c2ccc(-c3ccc4c(c3)C(C)(C)c3cccc5ccc(N(c6ccc(-c7ccccc7)cc6)c6ccc(-c7ccccc7)cc6)c-4c35)cc21]

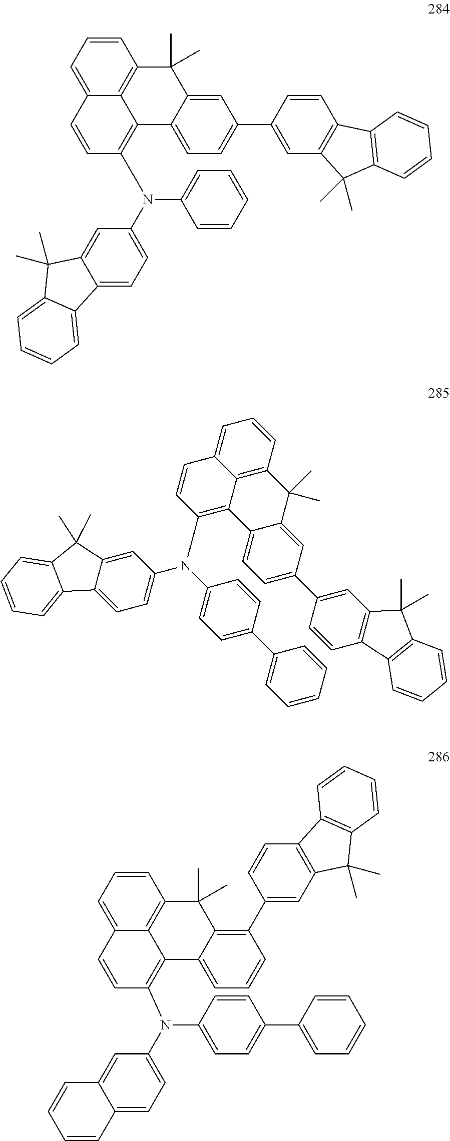 [SMILES: CC1(C)c2ccccc2-c2ccc(-c3ccc4c(c3)C(C)(C)c3cccc5ccc(N(c6ccccc6)c6ccc7c(c6)C(C)(C)c6ccccc6-7)c-4c35)cc21 ; CC1(C)c2ccccc2-c2ccc(-c3ccc4c(c3)C(C)(C)c3cccc5ccc(N(c6ccc(-c7ccccc7)cc6)c6ccc7c(c6)C(C)(C)c6ccccc6-7)c-4c35)cc21 ; CC1(C)c2ccccc2-c2ccc(-c3cccc4c3C(C)(C)c3cccc5ccc(N(c6ccc(-c7ccccc7)cc6)c6ccc7ccccc7c6)c-4c35)cc21]

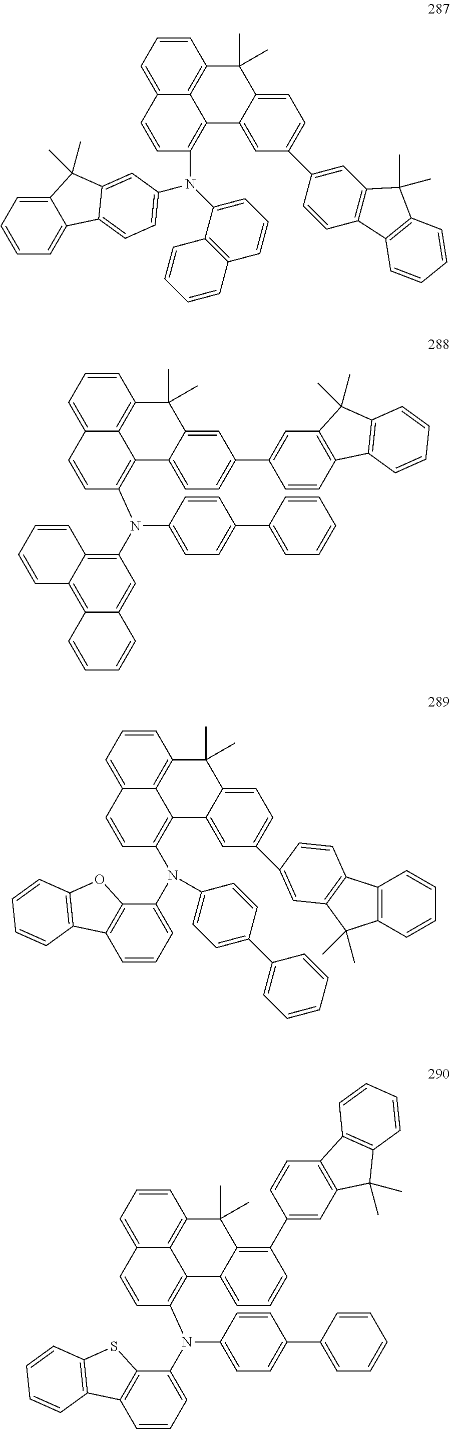 [SMILES: CC1(C)c2ccccc2-c2ccc(-c3ccc4c(c3)-c3c(N(c5ccc6c(c5)C(C)(C)c5ccccc5-6)c5cccc6ccccc56)ccc5cccc(c35)C4(C)C)cc21 ; CC1(C)c2ccccc2-c2ccc(-c3ccc4c(c3)C(C)(C)c3cccc5ccc(N(c6ccc(-c7ccccc7)cc6)c6cc7ccccc7c7ccccc67)c-4c35)cc21 ; CC1(C)c2ccccc2-c2ccc(-c3ccc4c(c3)-c3c(N(c5ccc(-c6ccccc6)cc5)c5cccc6c5oc5ccccc56)ccc5cccc(c35)C4(C)C)cc21 ; CC1(C)c2ccccc2-c2ccc(-c3cccc4c3C(C)(C)c3cccc5ccc(N(c6ccc(-c7ccccc7)cc6)c6cccc7c6sc6ccccc67)c-4c35)cc21]

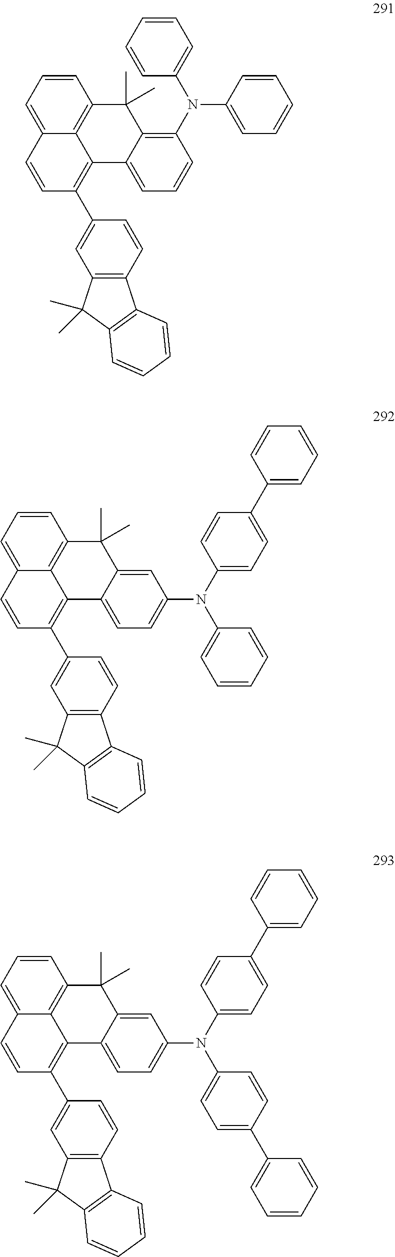 [SMILES: CC1(C)c2ccccc2-c2ccc(-c3ccc4cccc5c4c3-c3cccc(N(c4ccccc4)c4ccccc4)c3C5(C)C)cc21 ; CC1(C)c2ccccc2-c2ccc(-c3ccc4cccc5c4c3-c3ccc(N(c4ccccc4)c4ccc(-c6ccccc6)cc4)cc3C5(C)C)cc21 ; CC1(C)c2ccccc2-c2ccc(-c3ccc4cccc5c4c3-c3ccc(N(c4ccc(-c6ccccc6)cc4)c4ccc(-c6ccccc6)cc4)cc3C5(C)C)cc21]

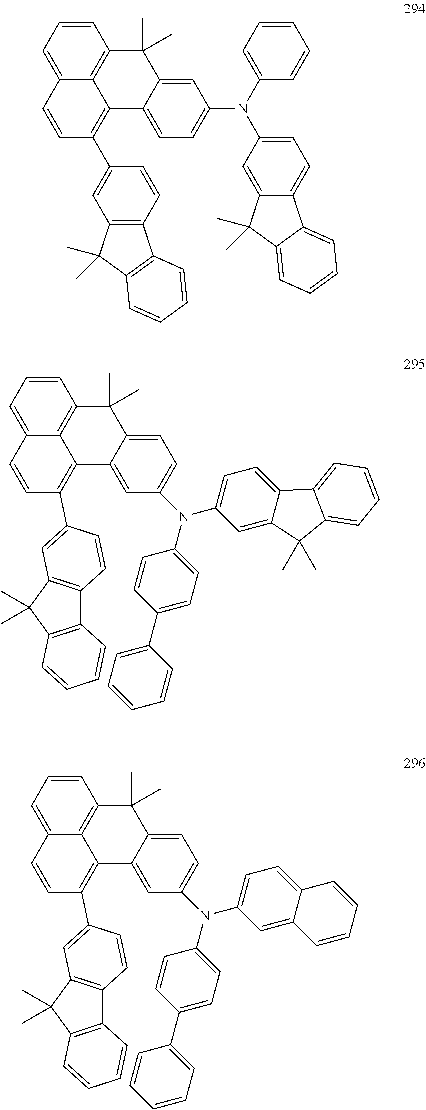 [SMILES: CC1(C)c2ccccc2-c2ccc(-c3ccc4cccc5c4c3-c3ccc(N(c4ccccc4)c4ccc6c(c4)C(C)(C)c4ccccc4-6)cc3C5(C)C)cc21 ; CC1(C)c2ccccc2-c2ccc(-c3ccc4cccc5c4c3-c3cc(N(c4ccc(-c6ccccc6)cc4)c4ccc6c(c4)C(C)(C)c4ccccc4-6)ccc3C5(C)C)cc21 ; CC1(C)c2ccccc2-c2ccc(-c3ccc4cccc5c4c3-c3cc(N(c4ccc(-c6ccccc6)cc4)c4ccc6ccccc6c4)ccc3C5(C)C)cc21]

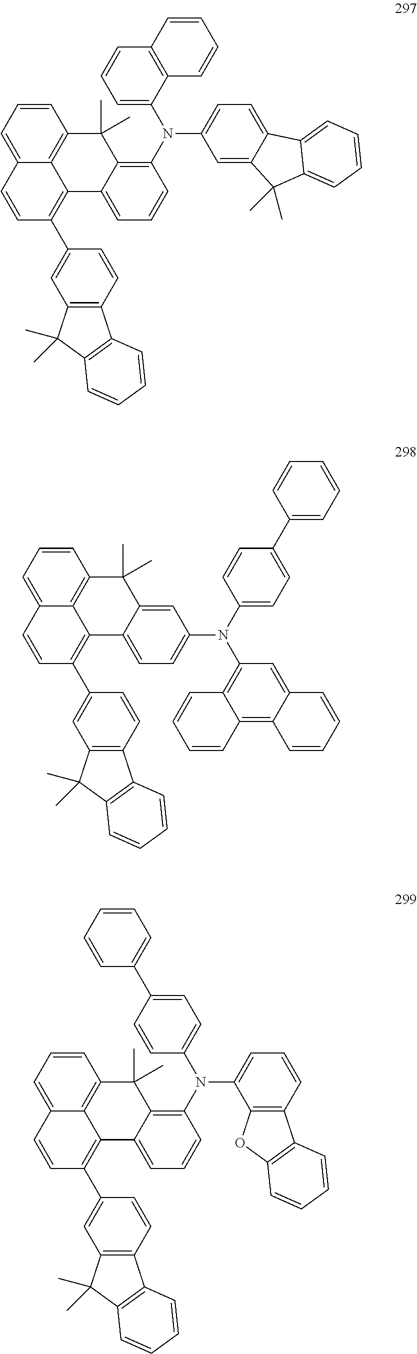 [SMILES: CC1(C)c2ccccc2-c2ccc(-c3ccc4cccc5c4c3-c3cccc(N(c4ccc6c(c4)C(C)(C)c4ccccc4-6)c4cccc6ccccc46)c3C5(C)C)cc21 ; CC1(C)c2ccccc2-c2ccc(-c3ccc4cccc5c4c3-c3ccc(N(c4ccc(-c6ccccc6)cc4)c4cc6ccccc6c6ccccc46)cc3C5(C)C)cc21 ; CC1(C)c2ccccc2-c2ccc(-c3ccc4cccc5c4c3-c3cccc(N(c4ccc(-c6ccccc6)cc4)c4cccc6c4oc4ccccc46)c3C5(C)C)cc21]

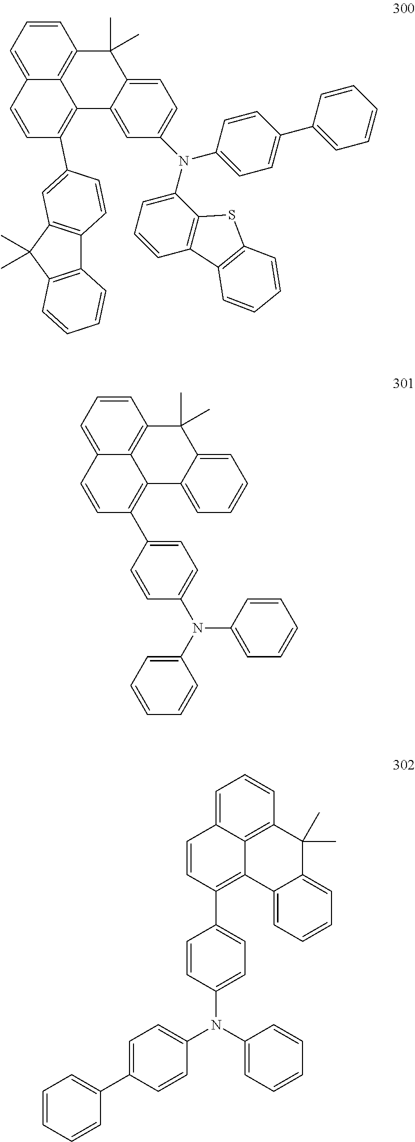 [SMILES: CC1(C)c2ccccc2-c2ccc(-c3ccc4cccc5c4c3-c3cc(N(c4ccc(-c6ccccc6)cc4)c4cccc6c4sc4ccccc46)ccc3C5(C)C)cc21 ; CC1(C)c2ccccc2-c2c(-c3ccc(N(c4ccccc4)c4ccccc4)cc3)ccc3cccc1c23 ; CC1(C)c2ccccc2-c2c(-c3ccc(N(c4ccccc4)c4ccc(-c5ccccc5)cc4)cc3)ccc3cccc1c23]

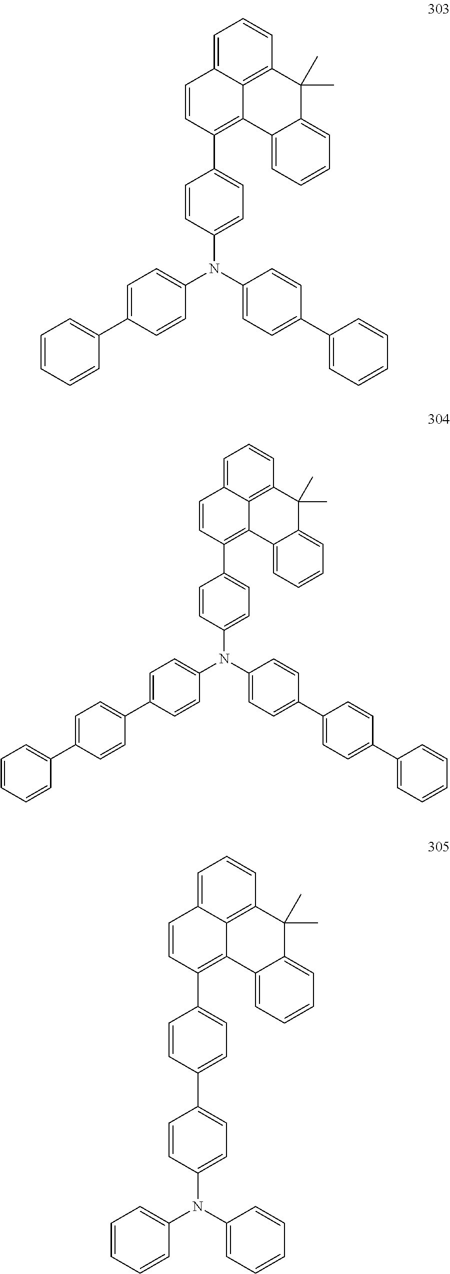 [SMILES: CC1(C)c2ccccc2-c2c(-c3ccc(N(c4ccc(-c5ccccc5)cc4)c4ccc(-c5ccccc5)cc4)cc3)ccc3cccc1c23 ; CC1(C)c2ccccc2-c2c(-c3ccc(N(c4ccc(-c5ccc(-c6ccccc6)cc5)cc4)c4ccc(-c5ccc(-c6ccccc6)cc5)cc4)cc3)ccc3cccc1c23 ; CC1(C)c2ccccc2-c2c(-c3ccc(-c4ccc(N(c5ccccc5)c5ccccc5)cc4)cc3)ccc3cccc1c23]

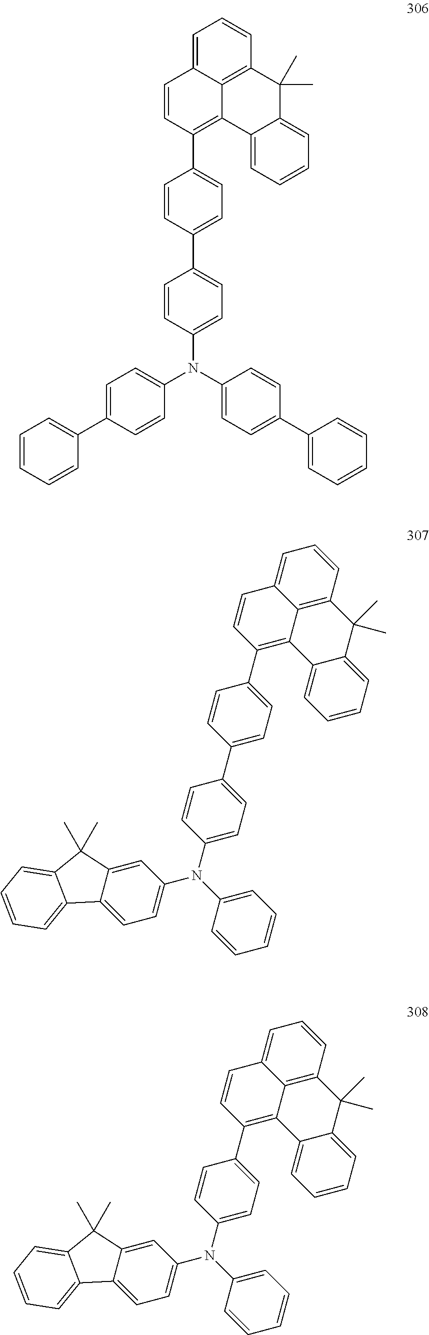 [SMILES: CC1(C)c2ccccc2-c2c(-c3ccc(-c4ccc(N(c5ccc(-c6ccccc6)cc5)c5ccc(-c6ccccc6)cc5)cc4)cc3)ccc3cccc1c23 ; CC1(C)c2ccccc2-c2ccc(N(c3ccccc3)c3ccc(-c4ccc(-c5ccc6cccc7c6c5-c5ccccc5C7(C)C)cc4)cc3)cc21 ; CC1(C)c2ccccc2-c2ccc(N(c3ccccc3)c3ccc(-c4ccc5cccc6c5c4-c4ccccc4C6(C)C)cc3)cc21]

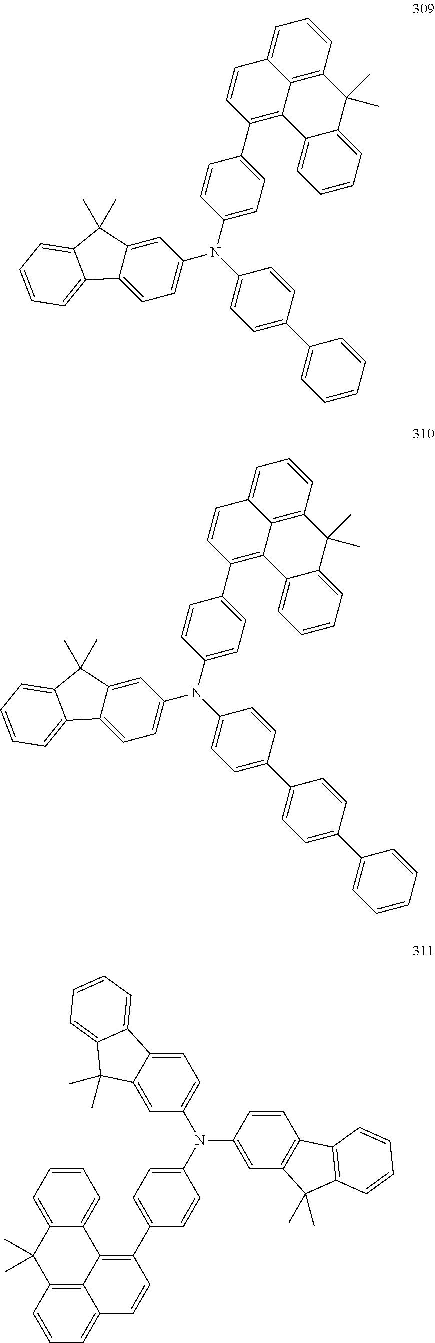

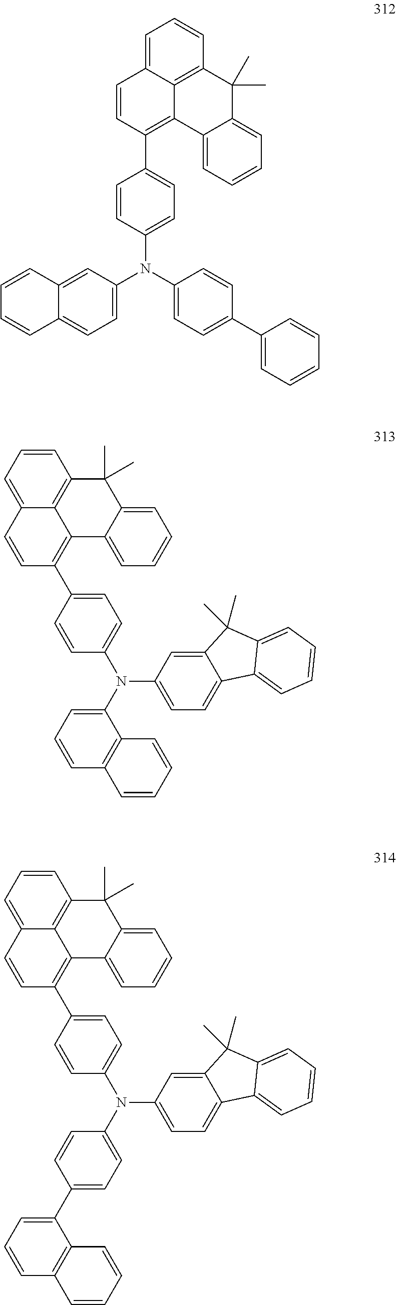 [SMILES: CC1(C)c2ccccc2-c2c(-c3ccc(N(c4ccc(-c5ccccc5)cc4)c4ccc5ccccc5c4)cc3)ccc3cccc1c23 ; CC1(C)c2ccccc2-c2ccc(N(c3ccc(-c4ccc5cccc6c5c4-c4ccccc4C6(C)C)cc3)c3cccc4ccccc34)cc21 ; CC1(C)c2ccccc2-c2ccc(N(c3ccc(-c4ccc5cccc6c5c4-c4ccccc4C6(C)C)cc3)c3ccc(-c4cccc5ccccc45)cc3)cc21]

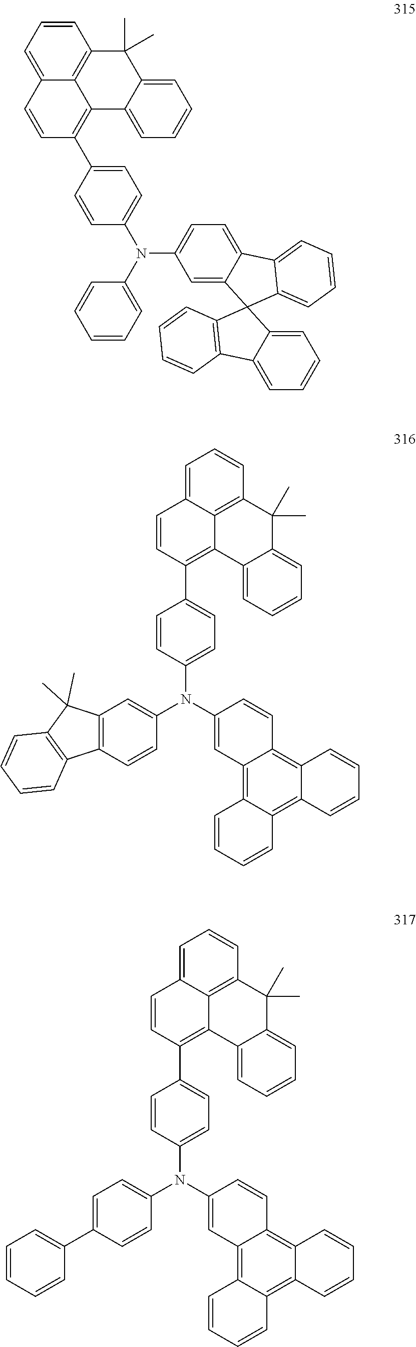 [SMILES: CC1(C)c2ccccc2-c2c(-c3ccc(N(c4ccccc4)c4ccc5c(c4)C4(c6ccccc6-c6ccccc64)c4ccccc4-5)cc3)ccc3cccc1c23 ; CC1(C)c2ccccc2-c2ccc(N(c3ccc(-c4ccc5cccc6c5c4-c4ccccc4C6(C)C)cc3)c3ccc4c5ccccc5c5ccccc5c4c3)cc21 ; CC1(C)c2ccccc2-c2c(-c3ccc(N(c4ccc(-c5ccccc5)cc4)c4ccc5c6ccccc6c6ccccc6c5c4)cc3)ccc3cccc1c23]

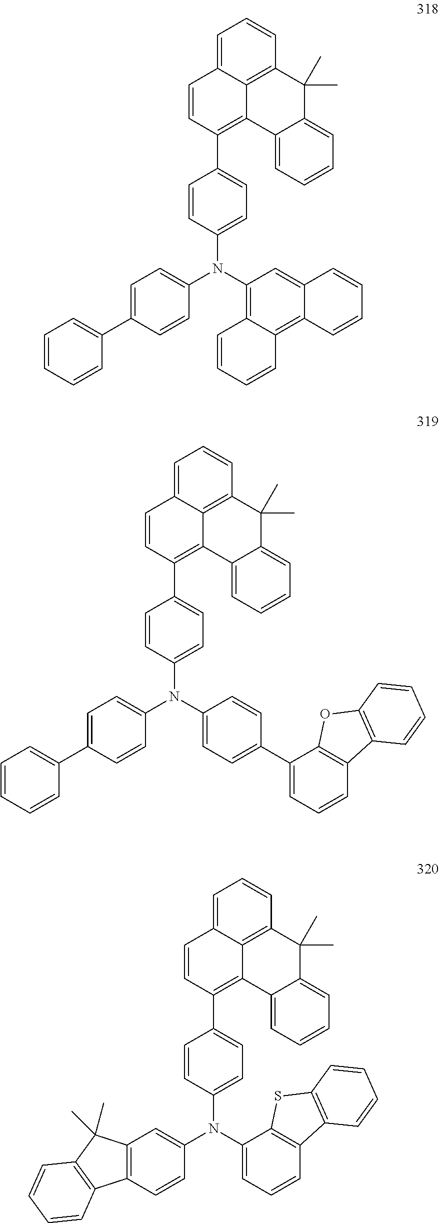 [SMILES: CC1(C)c2ccccc2-c2c(-c3ccc(N(c4ccc(-c5ccccc5)cc4)c4cc5ccccc5c5ccccc45)cc3)ccc3cccc1c23 ; CC1(C)c2ccccc2-c2c(-c3ccc(N(c4ccc(-c5ccccc5)cc4)c4ccc(-c5cccc6c5oc5ccccc56)cc4)cc3)ccc3cccc1c23 ; CC1(C)c2ccccc2-c2ccc(N(c3ccc(-c4ccc5cccc6c5c4-c4ccccc4C6(C)C)cc3)c3cccc4c3sc3ccccc34)cc21]

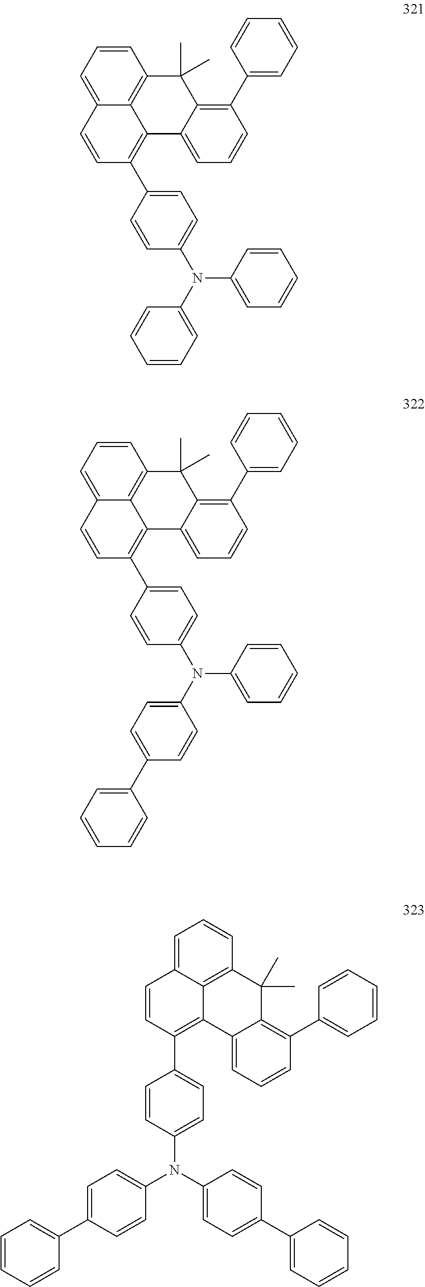 [SMILES: CC1(C)c2c(-c3ccccc3)cccc2-c2c(-c3ccc(N(c4ccccc4)c4ccccc4)cc3)ccc3cccc1c23 ; CC1(C)c2c(-c3ccccc3)cccc2-c2c(-c3ccc(N(c4ccccc4)c4ccc(-c5ccccc5)cc4)cc3)ccc3cccc1c23 ; CC1(C)c2c(-c3ccccc3)cccc2-c2c(-c3ccc(N(c4ccc(-c5ccccc5)cc4)c4ccc(-c5ccccc5)cc4)cc3)ccc3cccc1c23]

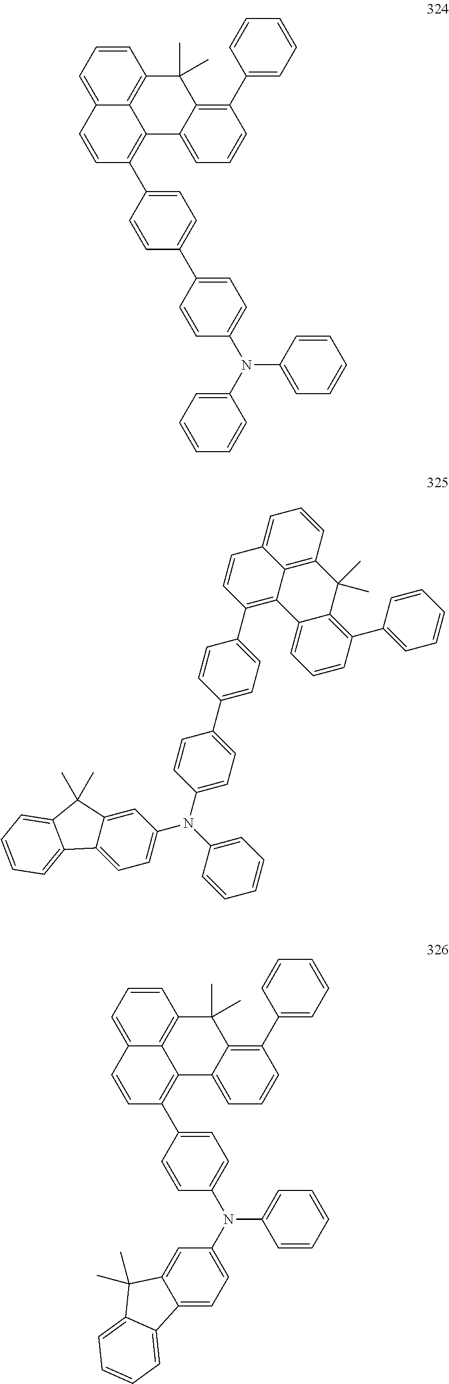 [SMILES: CC1(C)c2c(-c3ccccc3)cccc2-c2c(-c3ccc(-c4ccc(N(c5ccccc5)c5ccccc5)cc4)cc3)ccc3cccc1c23 ; CC1(C)c2ccccc2-c2ccc(N(c3ccccc3)c3ccc(-c4ccc(-c5ccc6cccc7c6c5-c5cccc(-c6ccccc6)c5C7(C)C)cc4)cc3)cc21 ; CC1(C)c2ccccc2-c2ccc(N(c3ccccc3)c3ccc(-c4ccc5cccc6c5c4-c4cccc(-c5ccccc5)c4C6(C)C)cc3)cc21]

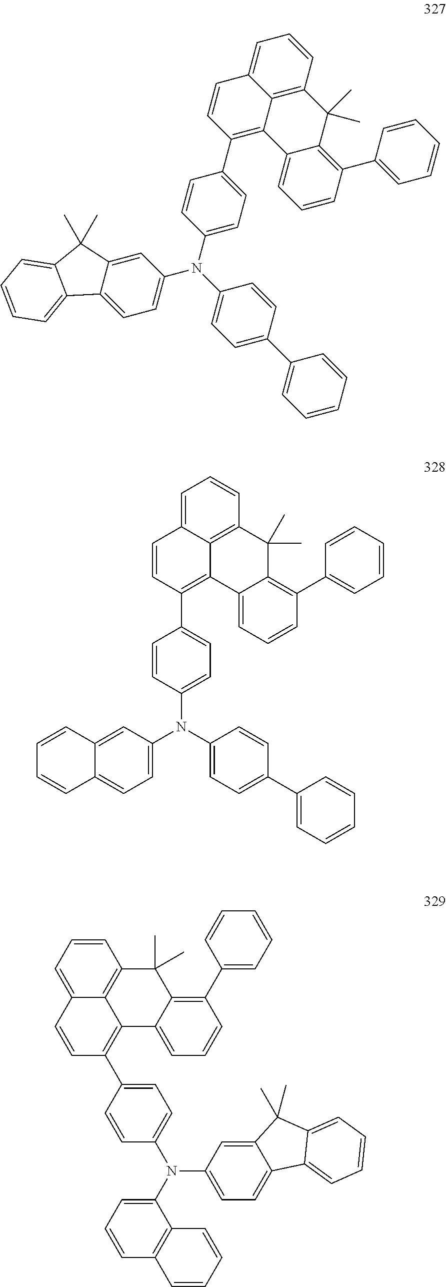 [SMILES: CC1(C)c2ccccc2-c2ccc(N(c3ccc(-c4ccccc4)cc3)c3ccc(-c4ccc5cccc6c5c4-c4cccc(-c5ccccc5)c4C6(C)C)cc3)cc21 ; CC1(C)c2c(-c3ccccc3)cccc2-c2c(-c3ccc(N(c4ccc(-c5ccccc5)cc4)c4ccc5ccccc5c4)cc3)ccc3cccc1c23 ; CC1(C)c2ccccc2-c2ccc(N(c3ccc(-c4ccc5cccc6c5c4-c4cccc(-c5ccccc5)c4C6(C)C)cc3)c3cccc4ccccc34)cc21]

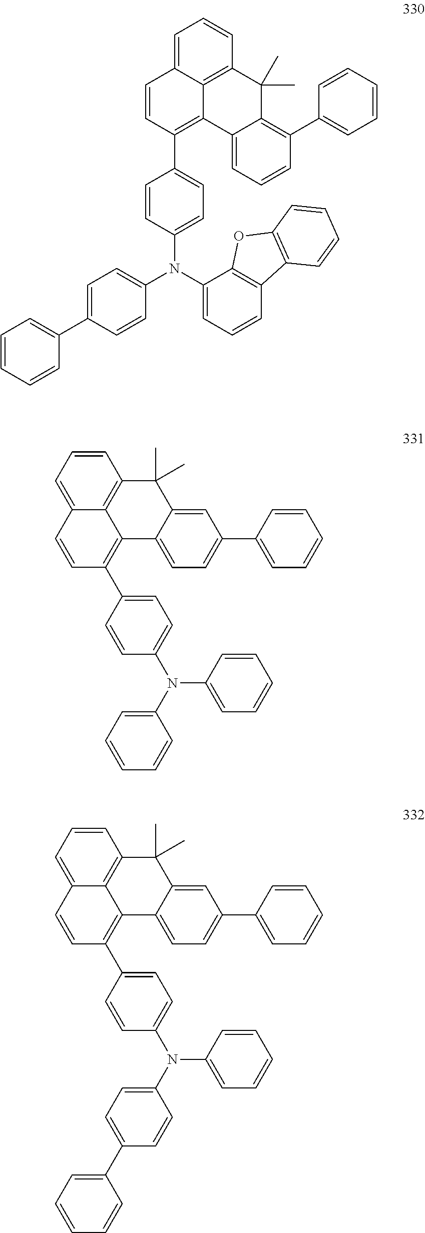 [SMILES: CC1(C)c2c(-c3ccccc3)cccc2-c2c(-c3ccc(N(c4ccc(-c5ccccc5)cc4)c4cccc5c4oc4ccccc45)cc3)ccc3cccc1c23 ; CC1(C)c2cc(-c3ccccc3)ccc2-c2c(-c3ccc(N(c4ccccc4)c4ccccc4)cc3)ccc3cccc1c23 ; CC1(C)c2cc(-c3ccccc3)ccc2-c2c(-c3ccc(N(c4ccccc4)c4ccc(-c5ccccc5)cc4)cc3)ccc3cccc1c23]

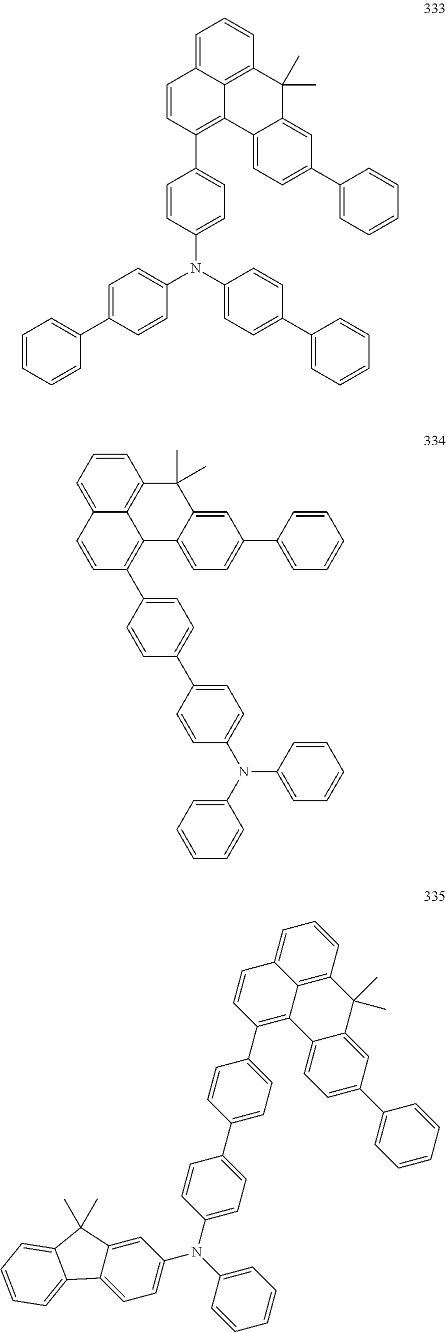 [SMILES: CC1(C)c2cc(-c3ccccc3)ccc2-c2c(-c3ccc(N(c4ccc(-c5ccccc5)cc4)c4ccc(-c5ccccc5)cc4)cc3)ccc3cccc1c23 ; CC1(C)c2cc(-c3ccccc3)ccc2-c2c(-c3ccc(-c4ccc(N(c5ccccc5)c5ccccc5)cc4)cc3)ccc3cccc1c23 ; CC1(C)c2ccccc2-c2ccc(N(c3ccccc3)c3ccc(-c4ccc(-c5ccc6cccc7c6c5-c5ccc(-c6ccccc6)cc5C7(C)C)cc4)cc3)cc21]

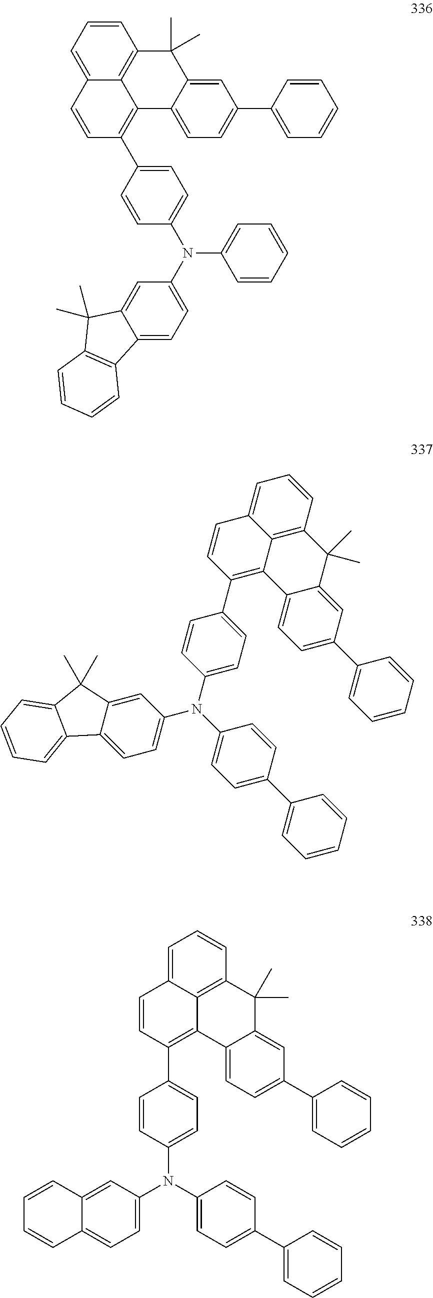 [SMILES: CC1(C)c2ccccc2-c2ccc(N(c3ccccc3)c3ccc(-c4ccc5cccc6c5c4-c4ccc(-c5ccccc5)cc4C6(C)C)cc3)cc21 ; CC1(C)c2ccccc2-c2ccc(N(c3ccc(-c4ccccc4)cc3)c3ccc(-c4ccc5cccc6c5c4-c4ccc(-c5ccccc5)cc4C6(C)C)cc3)cc21 ; CC1(C)c2cc(-c3ccccc3)ccc2-c2c(-c3ccc(N(c4ccc(-c5ccccc5)cc4)c4ccc5ccccc5c4)cc3)ccc3cccc1c23]

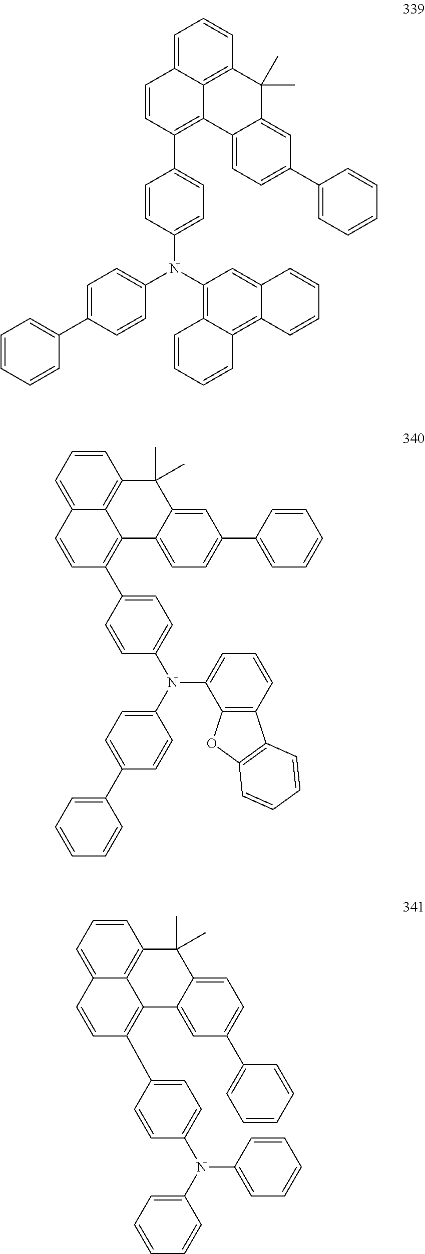 [SMILES: CC1(C)c2cc(-c3ccccc3)ccc2-c2c(-c3ccc(N(c4ccc(-c5ccccc5)cc4)c4cc5ccccc5c5ccccc45)cc3)ccc3cccc1c23 ; CC1(C)c2cc(-c3ccccc3)ccc2-c2c(-c3ccc(N(c4ccc(-c5ccccc5)cc4)c4cccc5c4oc4ccccc45)cc3)ccc3cccc1c23 ; CC1(C)c2ccc(-c3ccccc3)cc2-c2c(-c3ccc(N(c4ccccc4)c4ccccc4)cc3)ccc3cccc1c23]

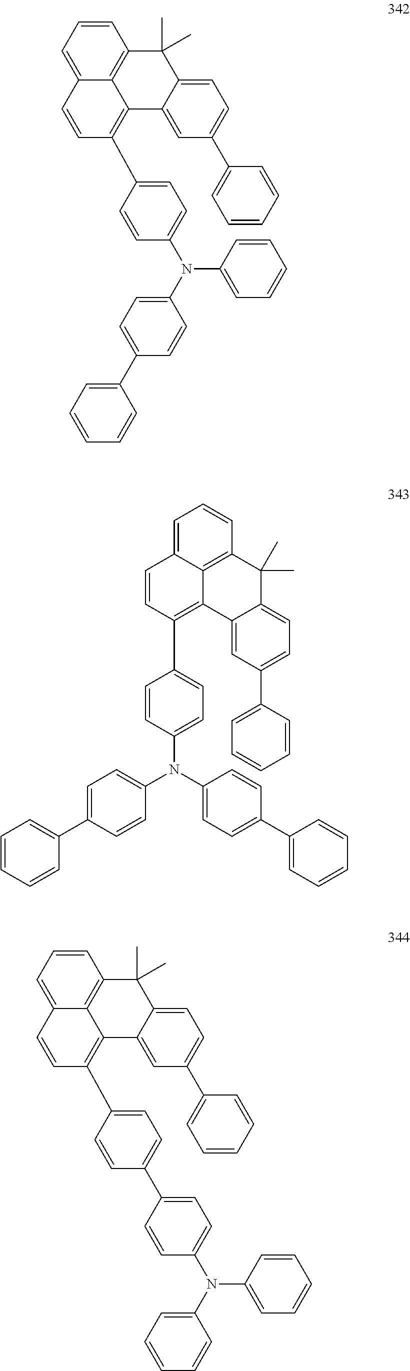 [SMILES: CC1(C)c2ccc(-c3ccccc3)cc2-c2c(-c3ccc(N(c4ccccc4)c4ccc(-c5ccccc5)cc4)cc3)ccc3cccc1c23 ; CC1(C)c2ccc(-c3ccccc3)cc2-c2c(-c3ccc(N(c4ccc(-c5ccccc5)cc4)c4ccc(-c5ccccc5)cc4)cc3)ccc3cccc1c23 ; CC1(C)c2ccc(-c3ccccc3)cc2-c2c(-c3ccc(-c4ccc(N(c5ccccc5)c5ccccc5)cc4)cc3)ccc3cccc1c23]

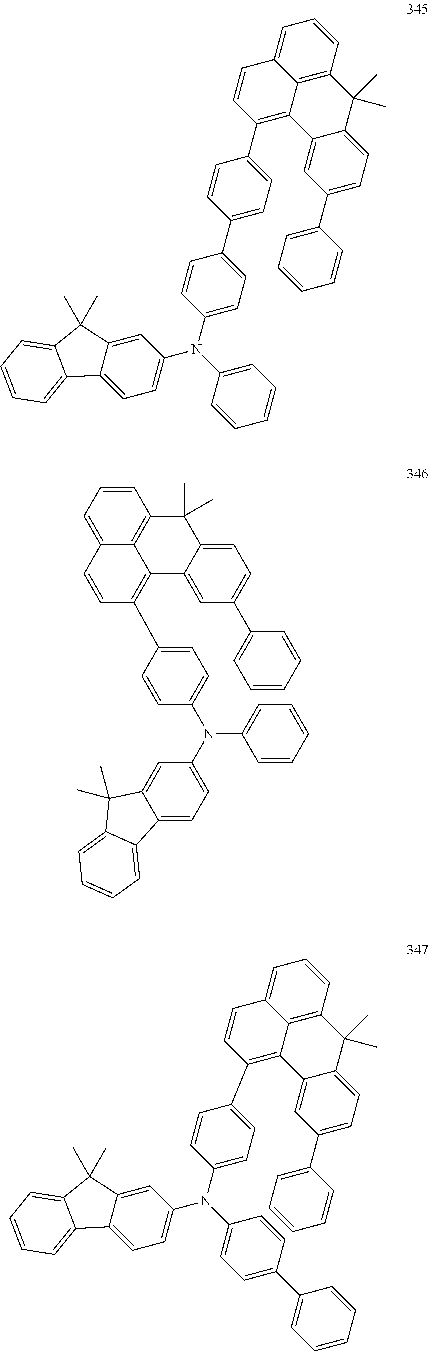

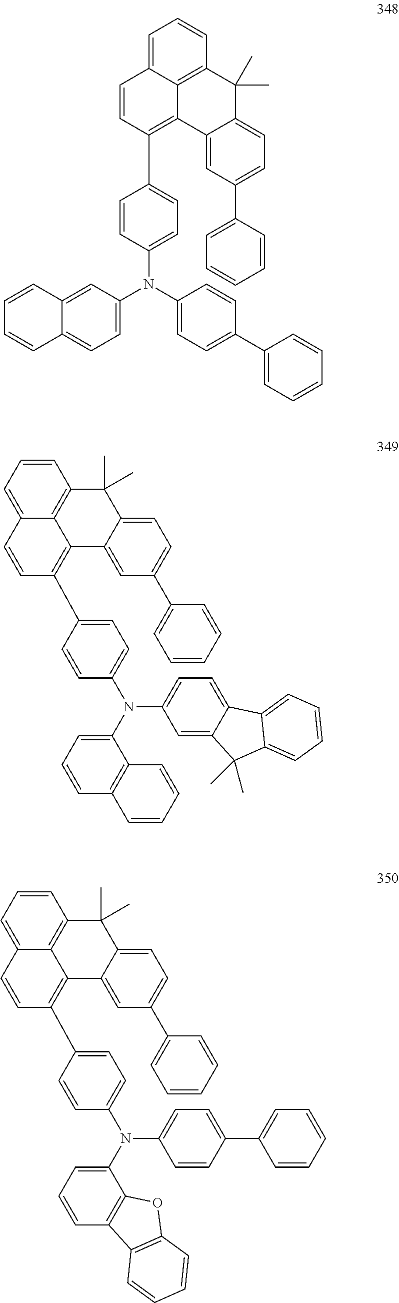 [SMILES: CC1(C)c2ccc(-c3ccccc3)cc2-c2c(-c3ccc(N(c4ccc(-c5ccccc5)cc4)c4ccc5ccccc5c4)cc3)ccc3cccc1c23 ; CC1(C)c2ccccc2-c2ccc(N(c3ccc(-c4ccc5cccc6c5c4-c4cc(-c5ccccc5)ccc4C6(C)C)cc3)c3cccc4ccccc34)cc21 ; CC1(C)c2ccc(-c3ccccc3)cc2-c2c(-c3ccc(N(c4ccc(-c5ccccc5)cc4)c4cccc5c4oc4ccccc45)cc3)ccc3cccc1c23]

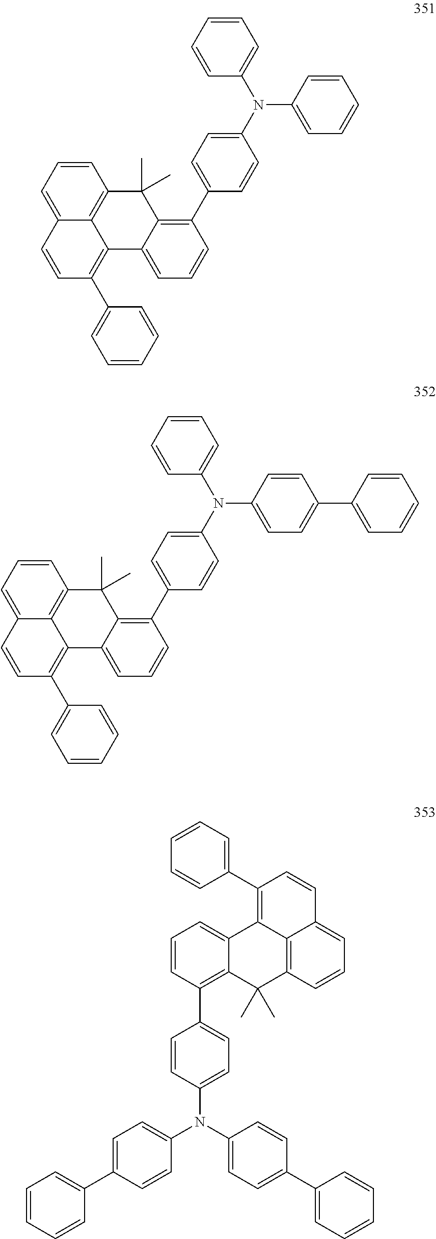 [SMILES: CC1(C)c2c(-c3ccc(N(c4ccccc4)c4ccccc4)cc3)cccc2-c2c(-c3ccccc3)ccc3cccc1c23 ; CC1(C)c2c(-c3ccc(N(c4ccccc4)c4ccc(-c5ccccc5)cc4)cc3)cccc2-c2c(-c3ccccc3)ccc3cccc1c23 ; CC1(C)c2c(-c3ccc(N(c4ccc(-c5ccccc5)cc4)c4ccc(-c5ccccc5)cc4)cc3)cccc2-c2c(-c3ccccc3)ccc3cccc1c23]

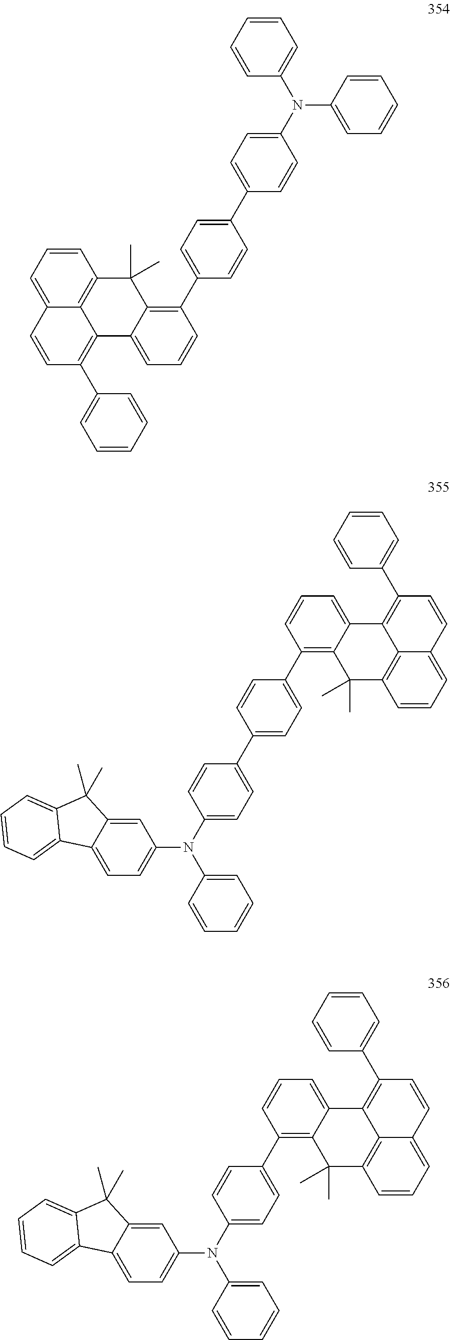 [SMILES: CC1(C)c2c(-c3ccc(-c4ccc(N(c5ccccc5)c5ccccc5)cc4)cc3)cccc2-c2c(-c3ccccc3)ccc3cccc1c23 ; CC1(C)c2ccccc2-c2ccc(N(c3ccccc3)c3ccc(-c4ccc(-c5cccc6c5C(C)(C)c5cccc7ccc(-c8ccccc8)c-6c57)cc4)cc3)cc21 ; CC1(C)c2ccccc2-c2ccc(N(c3ccccc3)c3ccc(-c4cccc5c4C(C)(C)c4cccc6ccc(-c7ccccc7)c-5c46)cc3)cc21]

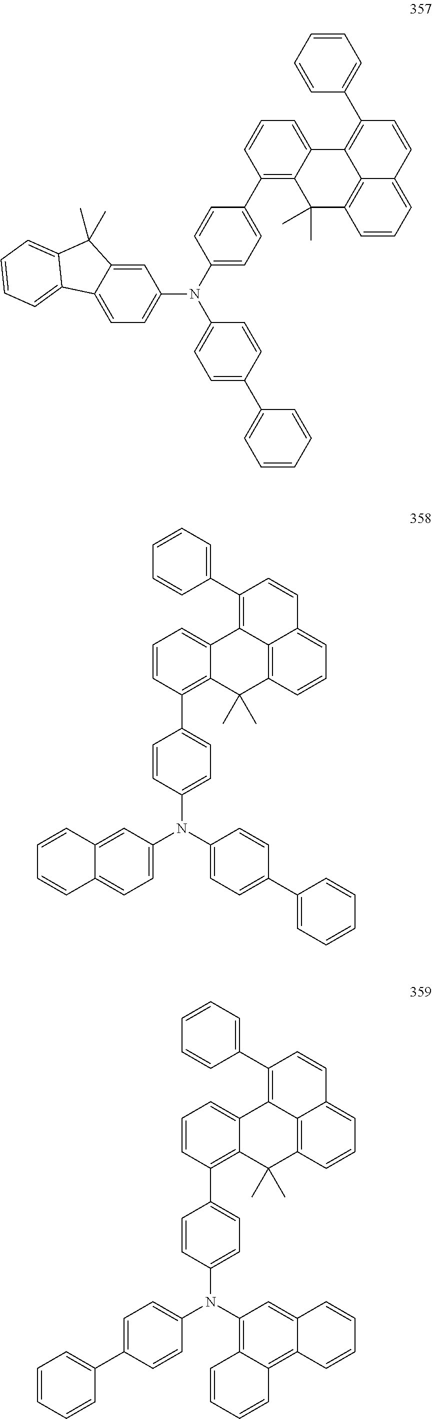 [SMILES: CC1(C)c2ccccc2-c2ccc(N(c3ccc(-c4ccccc4)cc3)c3ccc(-c4cccc5c4C(C)(C)c4cccc6ccc(-c7ccccc7)c-5c46)cc3)cc21 ; CC1(C)c2c(-c3ccc(N(c4ccc(-c5ccccc5)cc4)c4ccc5ccccc5c4)cc3)cccc2-c2c(-c3ccccc3)ccc3cccc1c23 ; CC1(C)c2c(-c3ccc(N(c4ccc(-c5ccccc5)cc4)c4cc5ccccc5c5ccccc45)cc3)cccc2-c2c(-c3ccccc3)ccc3cccc1c23]

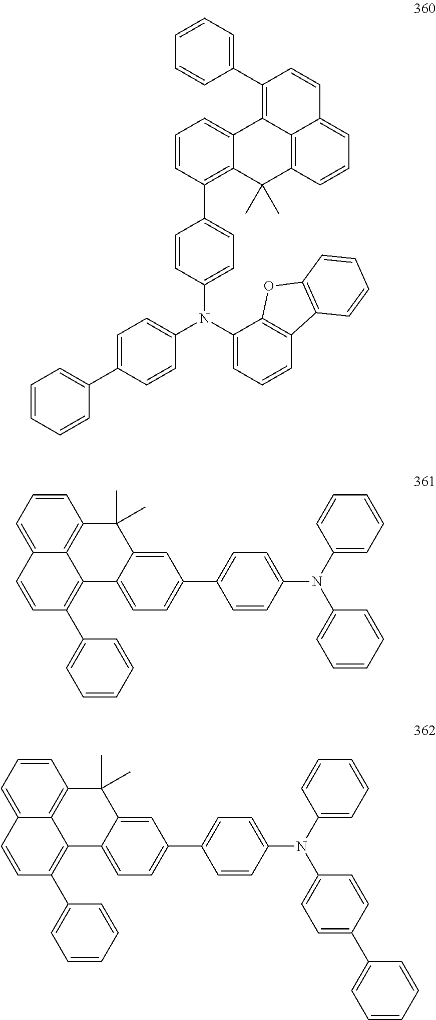 [SMILES: CC1(C)c2c(-c3ccc(N(c4ccc(-c5ccccc5)cc4)c4cccc5c4oc4ccccc45)cc3)cccc2-c2c(-c3ccccc3)ccc3cccc1c23 ; CC1(C)c2cc(-c3ccc(N(c4ccccc4)c4ccccc4)cc3)ccc2-c2c(-c3ccccc3)ccc3cccc1c23 ; CC1(C)c2cc(-c3ccc(N(c4ccccc4)c4ccc(-c5ccccc5)cc4)cc3)ccc2-c2c(-c3ccccc3)ccc3cccc1c23]

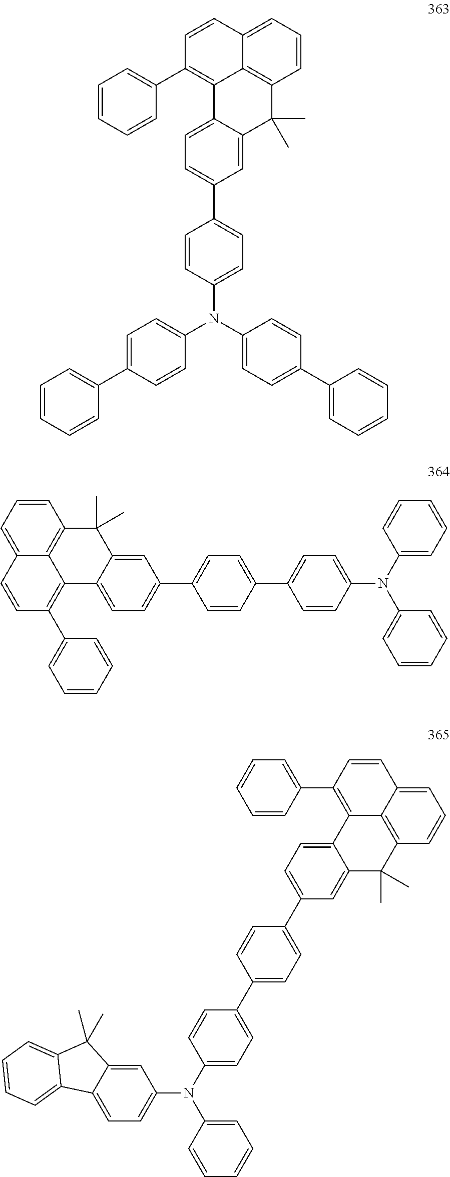 [SMILES: CC1(C)c2cc(-c3ccc(N(c4ccc(-c5ccccc5)cc4)c4ccc(-c5ccccc5)cc4)cc3)ccc2-c2c(-c3ccccc3)ccc3cccc1c23 ; CC1(C)c2cc(-c3ccc(-c4ccc(N(c5ccccc5)c5ccccc5)cc4)cc3)ccc2-c2c(-c3ccccc3)ccc3cccc1c23 ; CC1(C)c2ccccc2-c2ccc(N(c3ccccc3)c3ccc(-c4ccc(-c5ccc6c(c5)C(C)(C)c5cccc7ccc(-c8ccccc8)c-6c57)cc4)cc3)cc21]

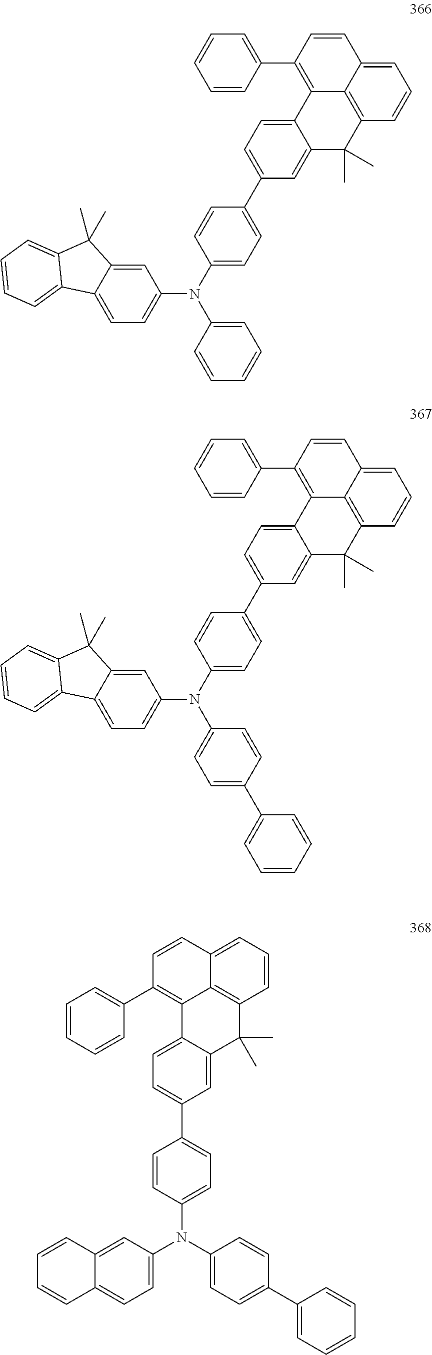 [SMILES: CC1(C)c2ccccc2-c2ccc(N(c3ccccc3)c3ccc(-c4ccc5c(c4)C(C)(C)c4cccc6ccc(-c7ccccc7)c-5c46)cc3)cc21 ; CC1(C)c2ccccc2-c2ccc(N(c3ccc(-c4ccccc4)cc3)c3ccc(-c4ccc5c(c4)C(C)(C)c4cccc6ccc(-c7ccccc7)c-5c46)cc3)cc21 ; CC1(C)c2cc(-c3ccc(N(c4ccc(-c5ccccc5)cc4)c4ccc5ccccc5c4)cc3)ccc2-c2c(-c3ccccc3)ccc3cccc1c23]

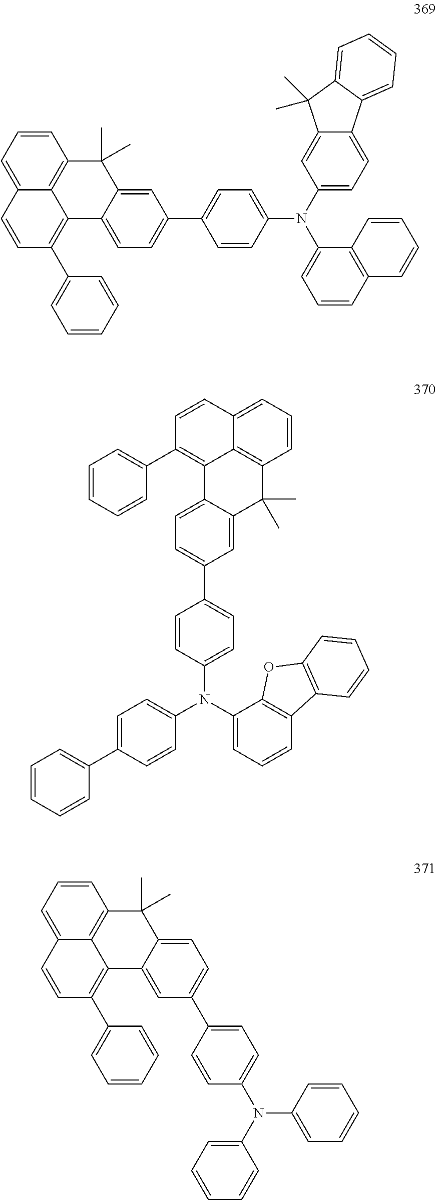 [SMILES: CC1(C)c2ccccc2-c2ccc(N(c3ccc(-c4ccc5c(c4)C(C)(C)c4cccc6ccc(-c7ccccc7)c-5c46)cc3)c3cccc4ccccc34)cc21 ; CC1(C)c2cc(-c3ccc(N(c4ccc(-c5ccccc5)cc4)c4cccc5c4oc4ccccc45)cc3)ccc2-c2c(-c3ccccc3)ccc3cccc1c23 ; CC1(C)c2ccc(-c3ccc(N(c4ccccc4)c4ccccc4)cc3)cc2-c2c(-c3ccccc3)ccc3cccc1c23]

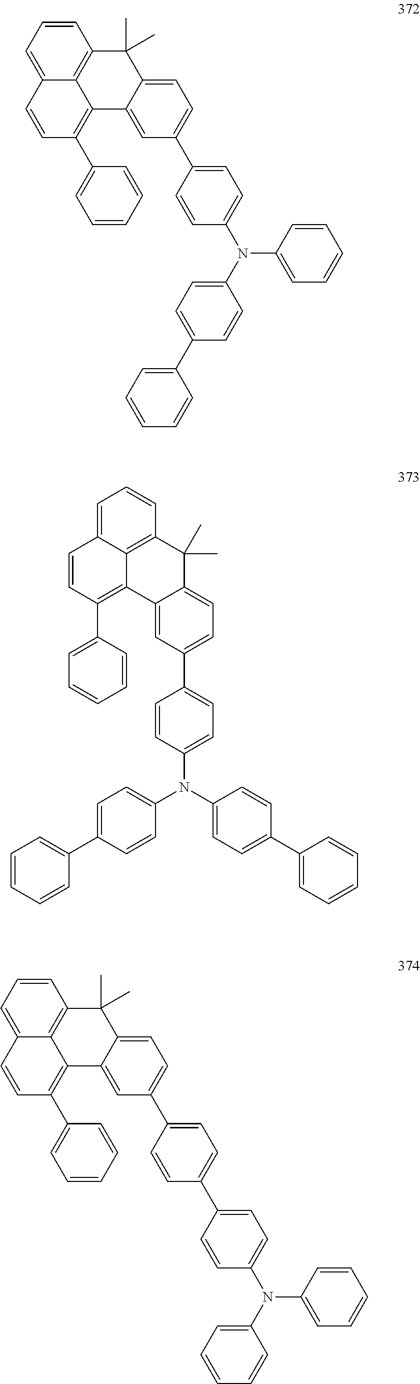 [SMILES: CC1(C)c2ccc(-c3ccc(N(c4ccccc4)c4ccc(-c5ccccc5)cc4)cc3)cc2-c2c(-c3ccccc3)ccc3cccc1c23 ; CC1(C)c2ccc(-c3ccc(N(c4ccc(-c5ccccc5)cc4)c4ccc(-c5ccccc5)cc4)cc3)cc2-c2c(-c3ccccc3)ccc3cccc1c23 ; CC1(C)c2ccc(-c3ccc(-c4ccc(N(c5ccccc5)c5ccccc5)cc4)cc3)cc2-c2c(-c3ccccc3)ccc3cccc1c23]

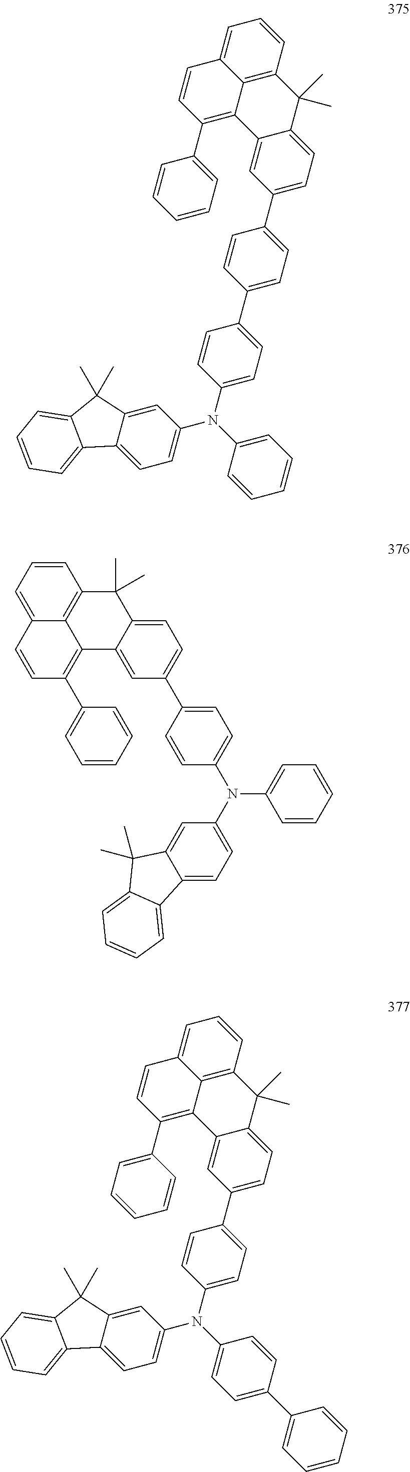 [SMILES: CC1(C)c2ccccc2-c2ccc(N(c3ccccc3)c3ccc(-c4ccc(-c5ccc6c(c5)-c5c(-c7ccccc7)ccc7cccc(c57)C6(C)C)cc4)cc3)cc21 ; CC1(C)c2ccccc2-c2ccc(N(c3ccccc3)c3ccc(-c4ccc5c(c4)-c4c(-c6ccccc6)ccc6cccc(c46)C5(C)C)cc3)cc21 ; CC1(C)c2ccccc2-c2ccc(N(c3ccc(-c4ccccc4)cc3)c3ccc(-c4ccc5c(c4)-c4c(-c6ccccc6)ccc6cccc(c46)C5(C)C)cc3)cc21]

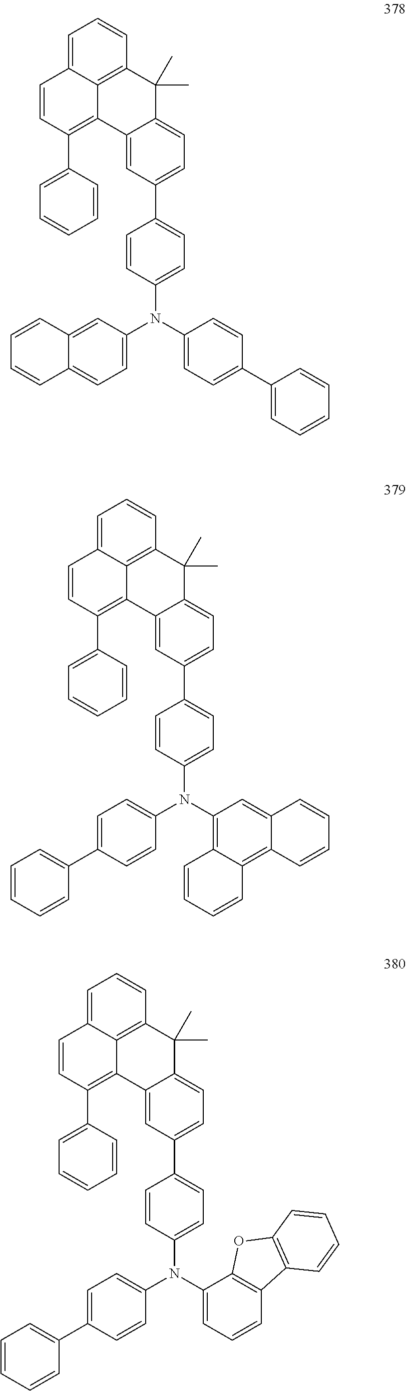 [SMILES: CC1(C)c2ccc(-c3ccc(N(c4ccc(-c5ccccc5)cc4)c4ccc5ccccc5c4)cc3)cc2-c2c(-c3ccccc3)ccc3cccc1c23 ; CC1(C)c2ccc(-c3ccc(N(c4ccc(-c5ccccc5)cc4)c4cc5ccccc5c5ccccc45)cc3)cc2-c2c(-c3ccccc3)ccc3cccc1c23 ; CC1(C)c2ccc(-c3ccc(N(c4ccc(-c5ccccc5)cc4)c4cccc5c4oc4ccccc45)cc3)cc2-c2c(-c3ccccc3)ccc3cccc1c23]

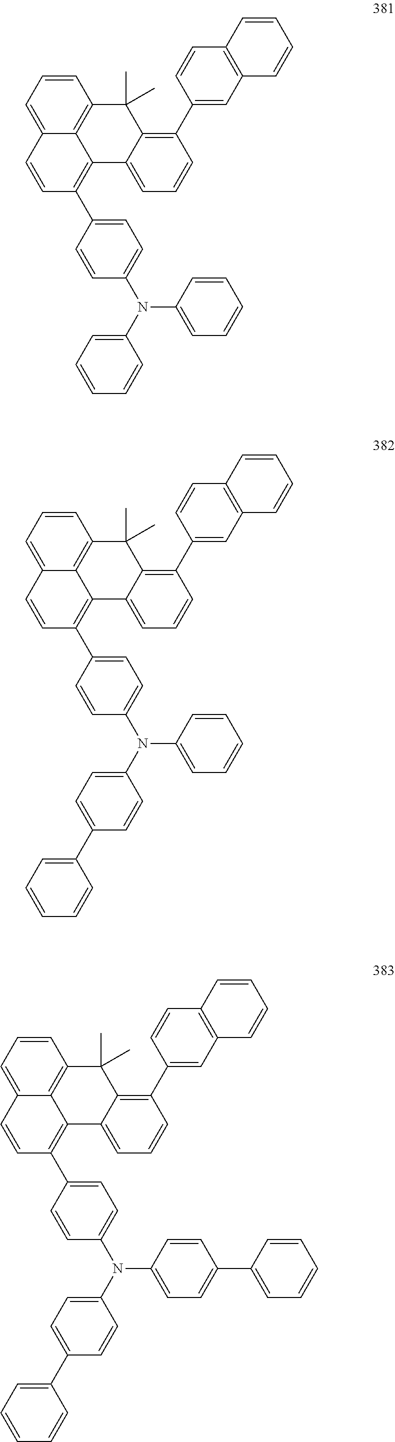 [SMILES: CC1(C)c2c(-c3ccc4ccccc4c3)cccc2-c2c(-c3ccc(N(c4ccccc4)c4ccccc4)cc3)ccc3cccc1c23 ; CC1(C)c2c(-c3ccc4ccccc4c3)cccc2-c2c(-c3ccc(N(c4ccccc4)c4ccc(-c5ccccc5)cc4)cc3)ccc3cccc1c23 ; CC1(C)c2c(-c3ccc4ccccc4c3)cccc2-c2c(-c3ccc(N(c4ccc(-c5ccccc5)cc4)c4ccc(-c5ccccc5)cc4)cc3)ccc3cccc1c23]

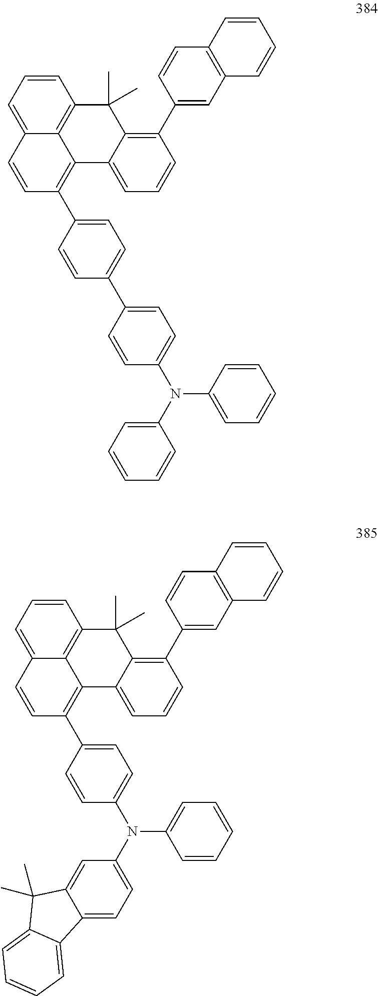 [SMILES: CC1(C)c2c(-c3ccc4ccccc4c3)cccc2-c2c(-c3ccc(-c4ccc(N(c5ccccc5)c5ccccc5)cc4)cc3)ccc3cccc1c23 ; CC1(C)c2ccccc2-c2ccc(N(c3ccccc3)c3ccc(-c4ccc5cccc6c5c4-c4cccc(-c5ccc7ccccc7c5)c4C6(C)C)cc3)cc21]

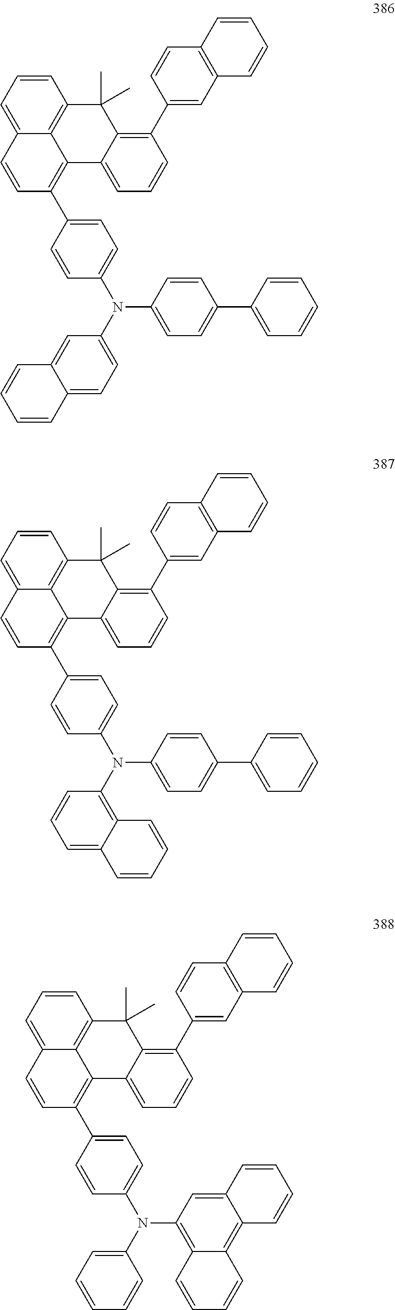 [SMILES: CC1(C)c2c(-c3ccc4ccccc4c3)cccc2-c2c(-c3ccc(N(c4ccc(-c5ccccc5)cc4)c4ccc5ccccc5c4)cc3)ccc3cccc1c23 ; CC1(C)c2c(-c3ccc4ccccc4c3)cccc2-c2c(-c3ccc(N(c4ccc(-c5ccccc5)cc4)c4cccc5ccccc45)cc3)ccc3cccc1c23 ; CC1(C)c2c(-c3ccc4ccccc4c3)cccc2-c2c(-c3ccc(N(c4ccccc4)c4cc5ccccc5c5ccccc45)cc3)ccc3cccc1c23]

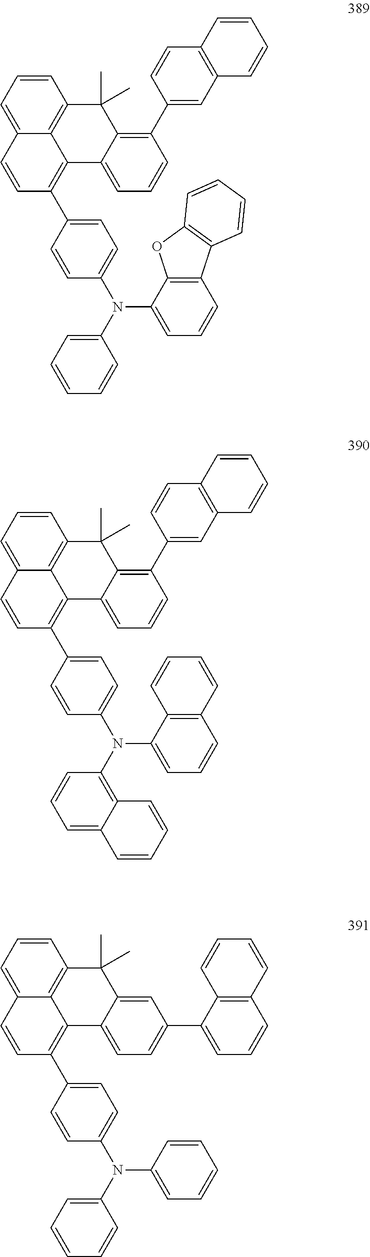 [SMILES: CC1(C)c2c(-c3ccc4ccccc4c3)cccc2-c2c(-c3ccc(N(c4ccccc4)c4cccc5c4oc4ccccc45)cc3)ccc3cccc1c23 ; CC1(C)c2c(-c3ccc4ccccc4c3)cccc2-c2c(-c3ccc(N(c4cccc5ccccc45)c4cccc5ccccc45)cc3)ccc3cccc1c23 ; CC1(C)c2cc(-c3cccc4ccccc34)ccc2-c2c(-c3ccc(N(c4ccccc4)c4ccccc4)cc3)ccc3cccc1c23]

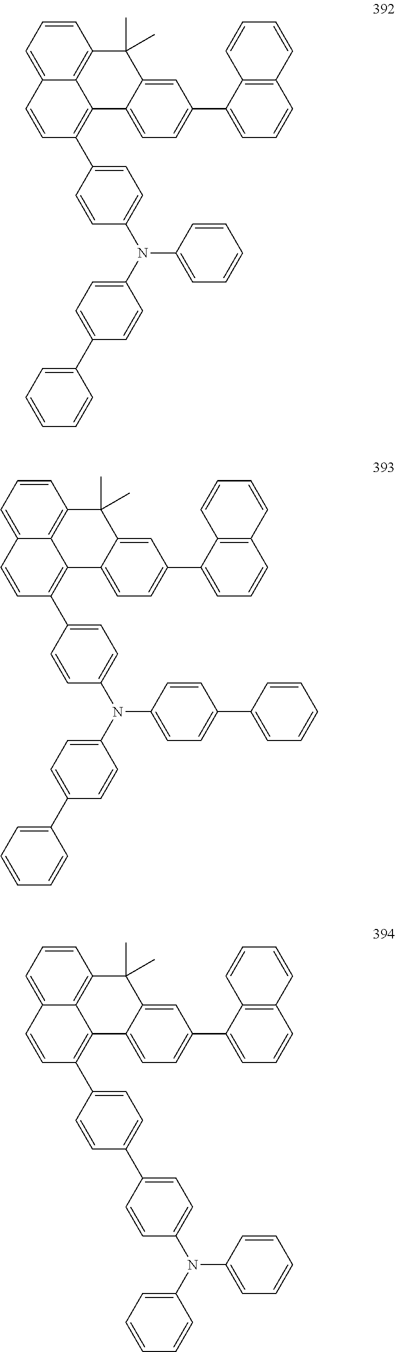 [SMILES: CC1(C)c2cc(-c3cccc4ccccc34)ccc2-c2c(-c3ccc(N(c4ccccc4)c4ccc(-c5ccccc5)cc4)cc3)ccc3cccc1c23 ; CC1(C)c2cc(-c3cccc4ccccc34)ccc2-c2c(-c3ccc(N(c4ccc(-c5ccccc5)cc4)c4ccc(-c5ccccc5)cc4)cc3)ccc3cccc1c23 ; CC1(C)c2cc(-c3cccc4ccccc34)ccc2-c2c(-c3ccc(-c4ccc(N(c5ccccc5)c5ccccc5)cc4)cc3)ccc3cccc1c23]

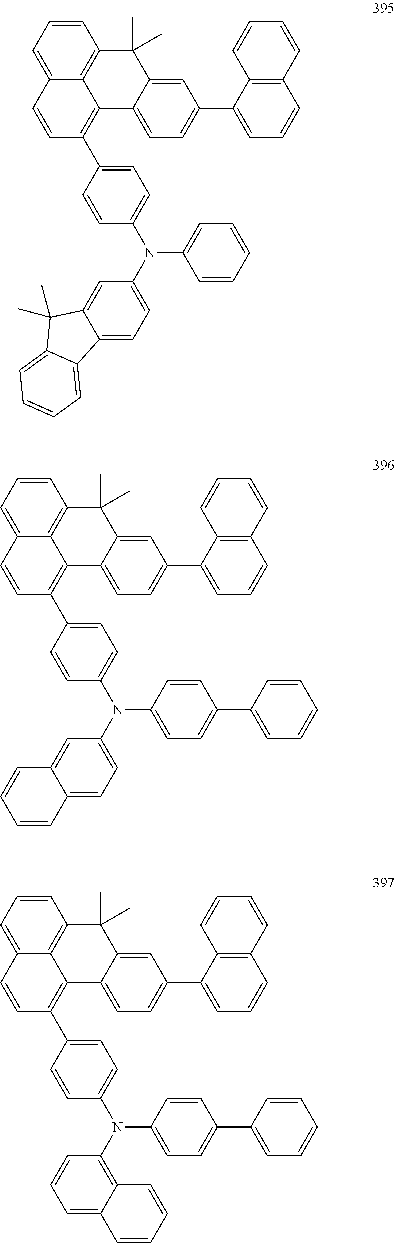 [SMILES: CC1(C)c2ccccc2-c2ccc(N(c3ccccc3)c3ccc(-c4ccc5cccc6c5c4-c4ccc(-c5cccc7ccccc57)cc4C6(C)C)cc3)cc21 ; CC1(C)c2cc(-c3cccc4ccccc34)ccc2-c2c(-c3ccc(N(c4ccc(-c5ccccc5)cc4)c4ccc5ccccc5c4)cc3)ccc3cccc1c23 ; CC1(C)c2cc(-c3cccc4ccccc34)ccc2-c2c(-c3ccc(N(c4ccc(-c5ccccc5)cc4)c4cccc5ccccc45)cc3)ccc3cccc1c23]

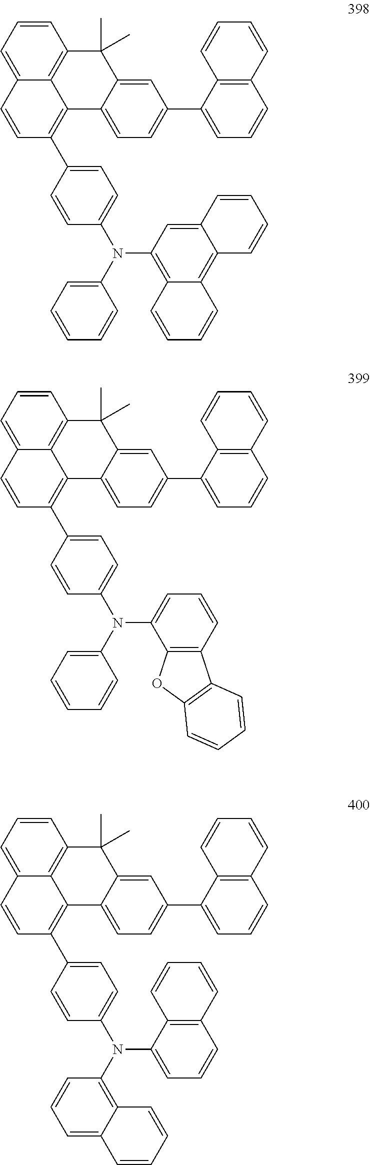 [SMILES: CC1(C)c2cc(-c3cccc4ccccc34)ccc2-c2c(-c3ccc(N(c4ccccc4)c4cc5ccccc5c5ccccc45)cc3)ccc3cccc1c23 ; CC1(C)c2cc(-c3cccc4ccccc34)ccc2-c2c(-c3ccc(N(c4ccccc4)c4cccc5c4oc4ccccc45)cc3)ccc3cccc1c23 ; CC1(C)c2cc(-c3cccc4ccccc34)ccc2-c2c(-c3ccc(N(c4cccc5ccccc45)c4cccc5ccccc45)cc3)ccc3cccc1c23]

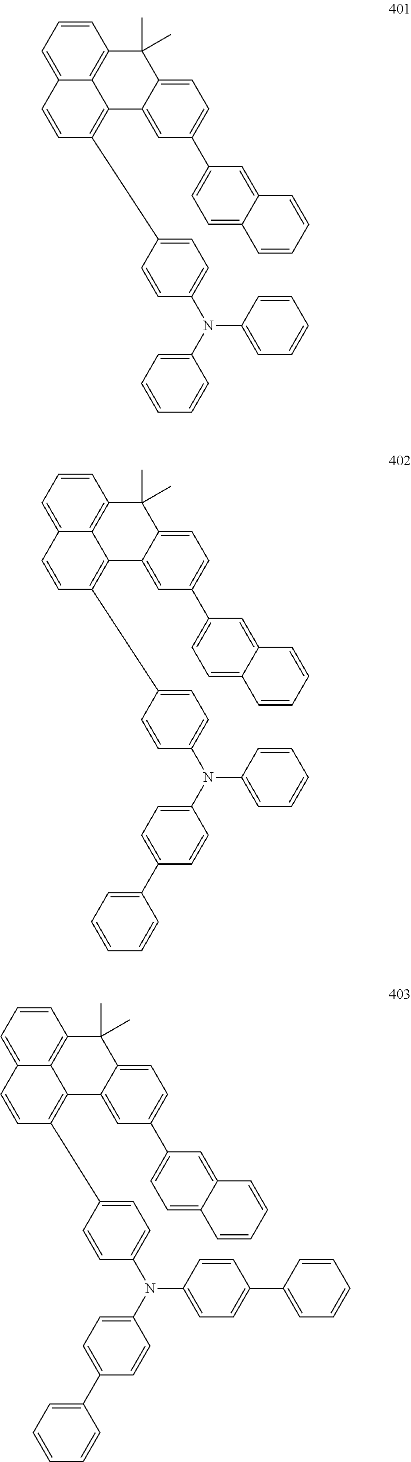 [SMILES: CC1(C)c2ccc(-c3ccc4ccccc4c3)cc2-c2c(-c3ccc(N(c4ccccc4)c4ccccc4)cc3)ccc3cccc1c23 ; CC1(C)c2ccc(-c3ccc4ccccc4c3)cc2-c2c(-c3ccc(N(c4ccccc4)c4ccc(-c5ccccc5)cc4)cc3)ccc3cccc1c23 ; CC1(C)c2ccc(-c3ccc4ccccc4c3)cc2-c2c(-c3ccc(N(c4ccc(-c5ccccc5)cc4)c4ccc(-c5ccccc5)cc4)cc3)ccc3cccc1c23]

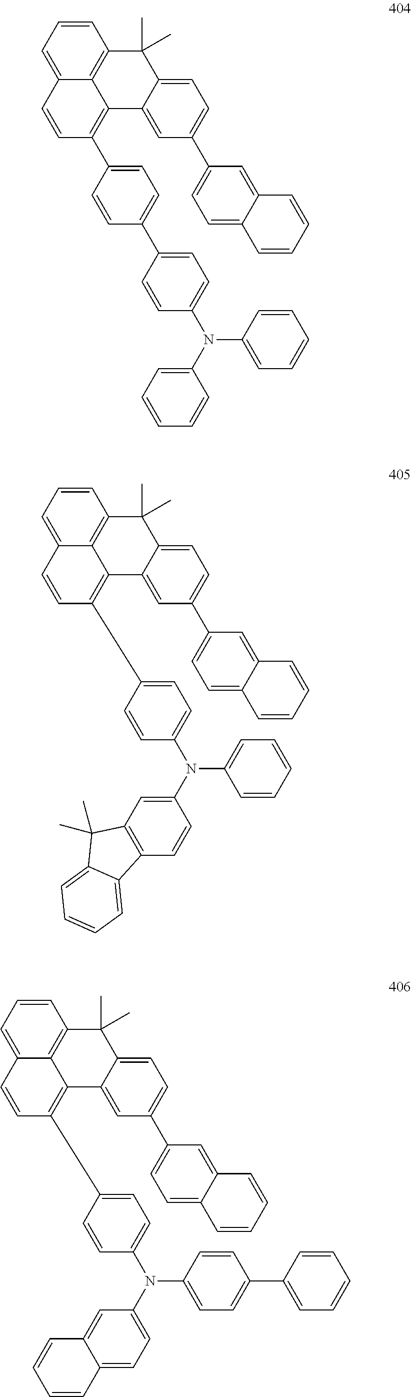 [SMILES: CC1(C)c2ccc(-c3ccc4ccccc4c3)cc2-c2c(-c3ccc(-c4ccc(N(c5ccccc5)c5ccccc5)cc4)cc3)ccc3cccc1c23 ; CC1(C)c2ccccc2-c2ccc(N(c3ccccc3)c3ccc(-c4ccc5cccc6c5c4-c4cc(-c5ccc7ccccc7c5)ccc4C6(C)C)cc3)cc21 ; CC1(C)c2ccc(-c3ccc4ccccc4c3)cc2-c2c(-c3ccc(N(c4ccc(-c5ccccc5)cc4)c4ccc5ccccc5c4)cc3)ccc3cccc1c23]

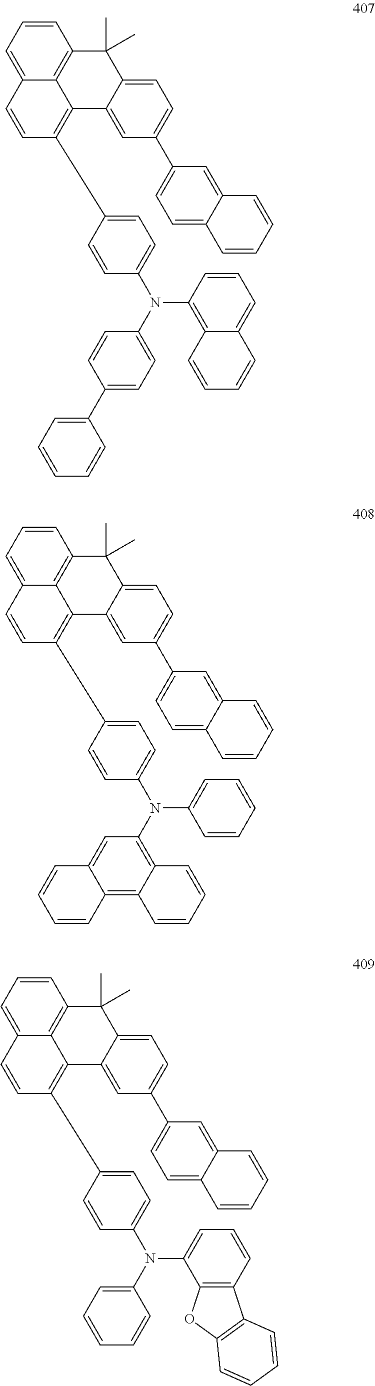 [SMILES: CC1(C)c2ccc(-c3ccc4ccccc4c3)cc2-c2c(-c3ccc(N(c4ccc(-c5ccccc5)cc4)c4cccc5ccccc45)cc3)ccc3cccc1c23 ; CC1(C)c2ccc(-c3ccc4ccccc4c3)cc2-c2c(-c3ccc(N(c4ccccc4)c4cc5ccccc5c5ccccc45)cc3)ccc3cccc1c23 ; CC1(C)c2ccc(-c3ccc4ccccc4c3)cc2-c2c(-c3ccc(N(c4ccccc4)c4cccc5c4oc4ccccc45)cc3)ccc3cccc1c23]

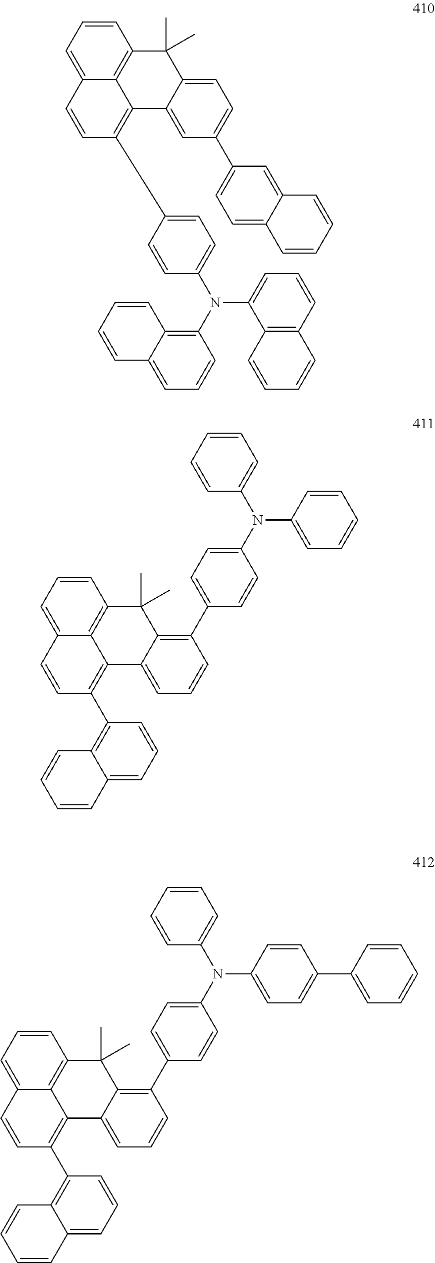

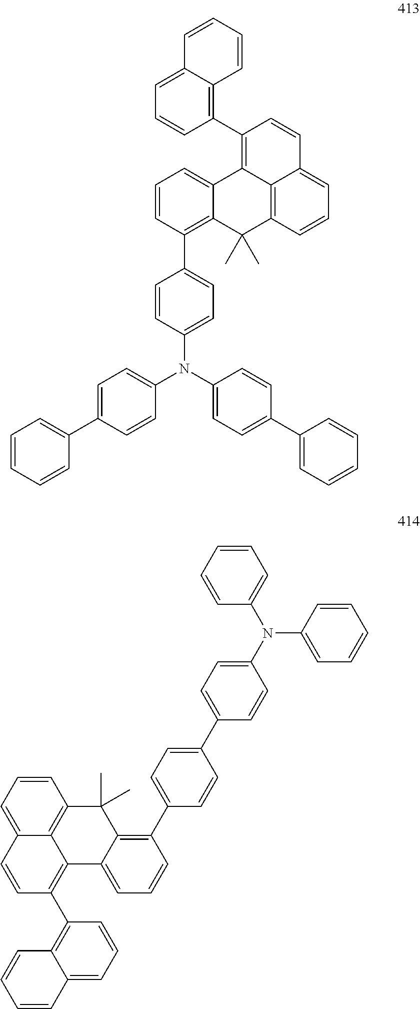 [SMILES: CC1(C)c2c(-c3ccc(N(c4ccc(-c5ccccc5)cc4)c4ccc(-c5ccccc5)cc4)cc3)cccc2-c2c(-c3cccc4ccccc34)ccc3cccc1c23 ; CC1(C)c2c(-c3ccc(-c4ccc(N(c5ccccc5)c5ccccc5)cc4)cc3)cccc2-c2c(-c3cccc4ccccc34)ccc3cccc1c23]

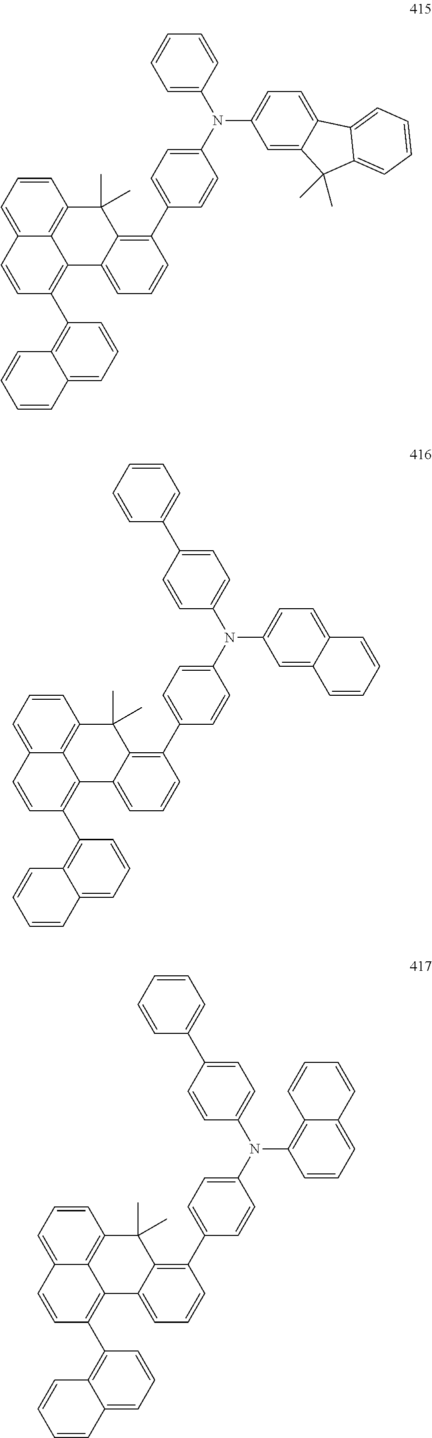 [SMILES: CC1(C)c2ccccc2-c2ccc(N(c3ccccc3)c3ccc(-c4cccc5c4C(C)(C)c4cccc6ccc(-c7cccc8ccccc78)c-5c46)cc3)cc21 ; CC1(C)c2c(-c3ccc(N(c4ccc(-c5ccccc5)cc4)c4ccc5ccccc5c4)cc3)cccc2-c2c(-c3cccc4ccccc34)ccc3cccc1c23 ; CC1(C)c2c(-c3ccc(N(c4ccc(-c5ccccc5)cc4)c4cccc5ccccc45)cc3)cccc2-c2c(-c3cccc4ccccc34)ccc3cccc1c23]

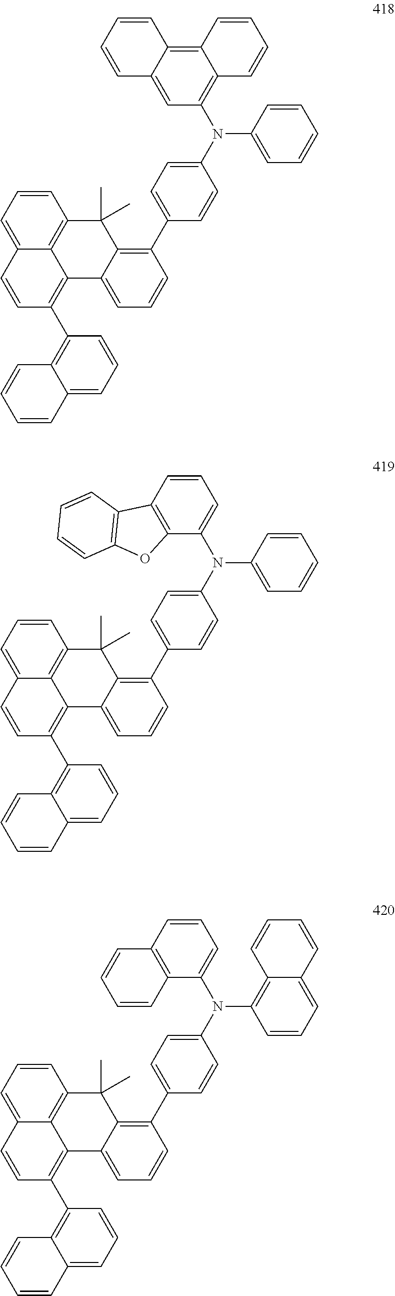 [SMILES: CC1(C)c2c(-c3ccc(N(c4ccccc4)c4cc5ccccc5c5ccccc45)cc3)cccc2-c2c(-c3cccc4ccccc34)ccc3cccc1c23 ; CC1(C)c2c(-c3ccc(N(c4ccccc4)c4cccc5c4oc4ccccc45)cc3)cccc2-c2c(-c3cccc4ccccc34)ccc3cccc1c23 ; CC1(C)c2c(-c3ccc(N(c4cccc5ccccc45)c4cccc5ccccc45)cc3)cccc2-c2c(-c3cccc4ccccc34)ccc3cccc1c23]

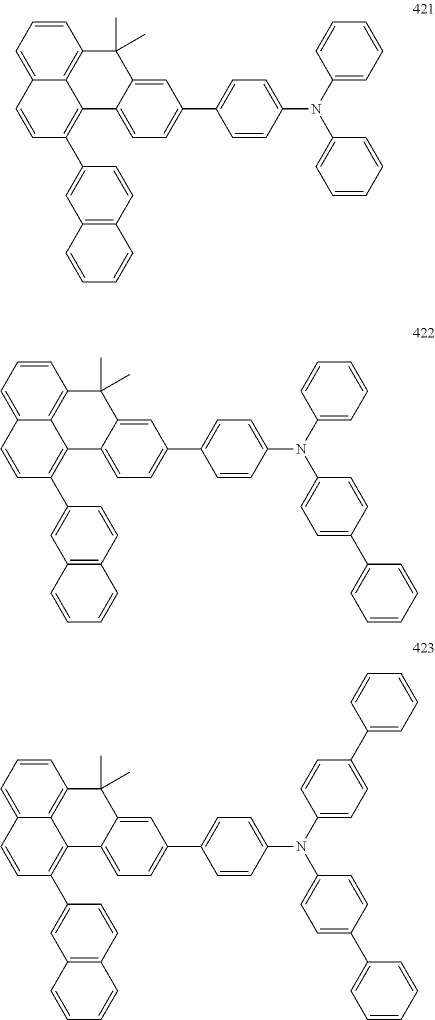 [SMILES: CC1(C)c2cc(-c3ccc(N(c4ccccc4)c4ccccc4)cc3)ccc2-c2c(-c3ccc4ccccc4c3)ccc3cccc1c23 ; CC1(C)c2cc(-c3ccc(N(c4ccccc4)c4ccc(-c5ccccc5)cc4)cc3)ccc2-c2c(-c3ccc4ccccc4c3)ccc3cccc1c23 ; CC1(C)c2cc(-c3ccc(N(c4ccc(-c5ccccc5)cc4)c4ccc(-c5ccccc5)cc4)cc3)ccc2-c2c(-c3ccc4ccccc4c3)ccc3cccc1c23]

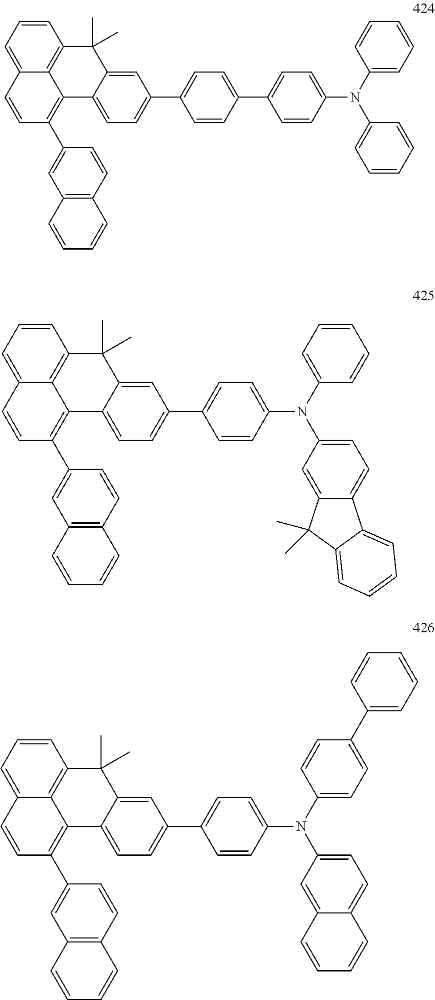 [SMILES: CC1(C)c2cc(-c3ccc(-c4ccc(N(c5ccccc5)c5ccccc5)cc4)cc3)ccc2-c2c(-c3ccc4ccccc4c3)ccc3cccc1c23 ; CC1(C)c2ccccc2-c2ccc(N(c3ccccc3)c3ccc(-c4ccc5c(c4)C(C)(C)c4cccc6ccc(-c7ccc8ccccc8c7)c-5c46)cc3)cc21 ; CC1(C)c2cc(-c3ccc(N(c4ccc(-c5ccccc5)cc4)c4ccc5ccccc5c4)cc3)ccc2-c2c(-c3ccc4ccccc4c3)ccc3cccc1c23]

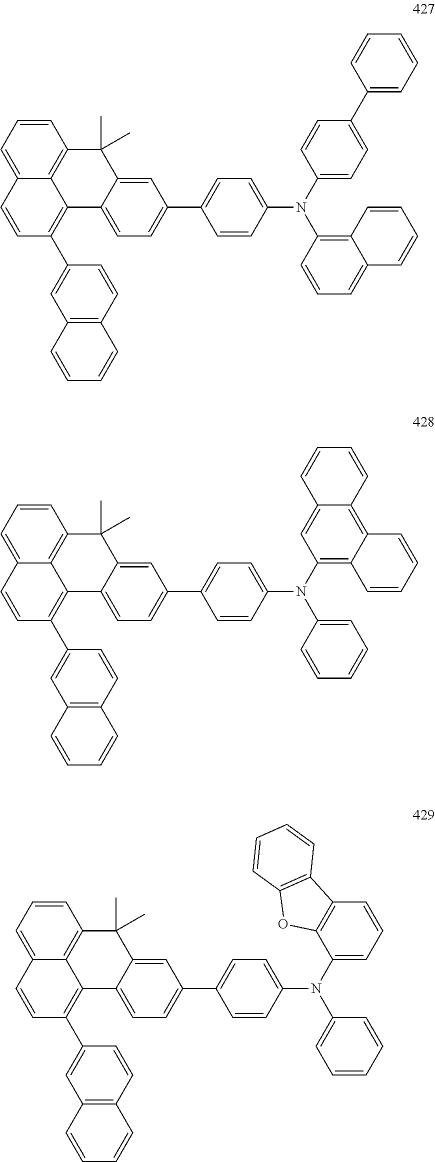 [SMILES: CC1(C)c2cc(-c3ccc(N(c4ccc(-c5ccccc5)cc4)c4cccc5ccccc45)cc3)ccc2-c2c(-c3ccc4ccccc4c3)ccc3cccc1c23 ; CC1(C)c2cc(-c3ccc(N(c4ccccc4)c4cc5ccccc5c5ccccc45)cc3)ccc2-c2c(-c3ccc4ccccc4c3)ccc3cccc1c23 ; CC1(C)c2cc(-c3ccc(N(c4ccccc4)c4cccc5c4oc4ccccc45)cc3)ccc2-c2c(-c3ccc4ccccc4c3)ccc3cccc1c23]

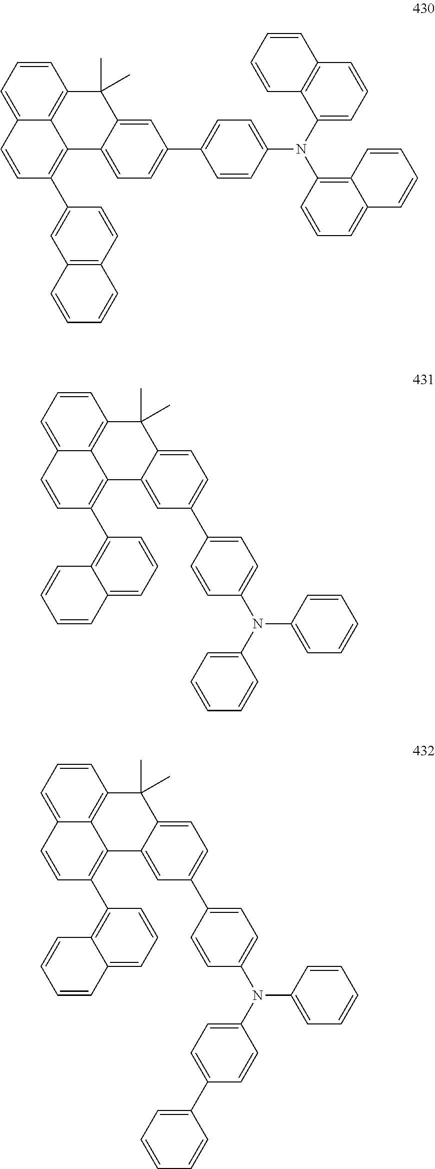 [SMILES: CC1(C)c2cc(-c3ccc(N(c4cccc5ccccc45)c4cccc5ccccc45)cc3)ccc2-c2c(-c3ccc4ccccc4c3)ccc3cccc1c23 ; CC1(C)c2ccc(-c3ccc(N(c4ccccc4)c4ccccc4)cc3)cc2-c2c(-c3cccc4ccccc34)ccc3cccc1c23 ; CC1(C)c2ccc(-c3ccc(N(c4ccccc4)c4ccc(-c5ccccc5)cc4)cc3)cc2-c2c(-c3cccc4ccccc34)ccc3cccc1c23]

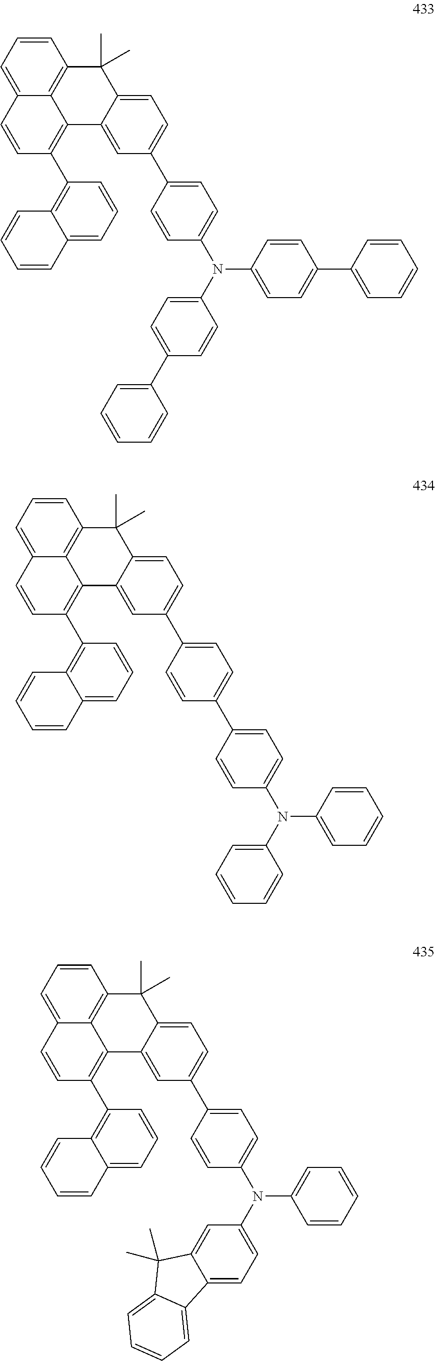 [SMILES: CC1(C)c2ccc(-c3ccc(N(c4ccc(-c5ccccc5)cc4)c4ccc(-c5ccccc5)cc4)cc3)cc2-c2c(-c3cccc4ccccc34)ccc3cccc1c23 ; CC1(C)c2ccc(-c3ccc(-c4ccc(N(c5ccccc5)c5ccccc5)cc4)cc3)cc2-c2c(-c3cccc4ccccc34)ccc3cccc1c23 ; CC1(C)c2ccccc2-c2ccc(N(c3ccccc3)c3ccc(-c4ccc5c(c4)-c4c(-c6cccc7ccccc67)ccc6cccc(c46)C5(C)C)cc3)cc21]

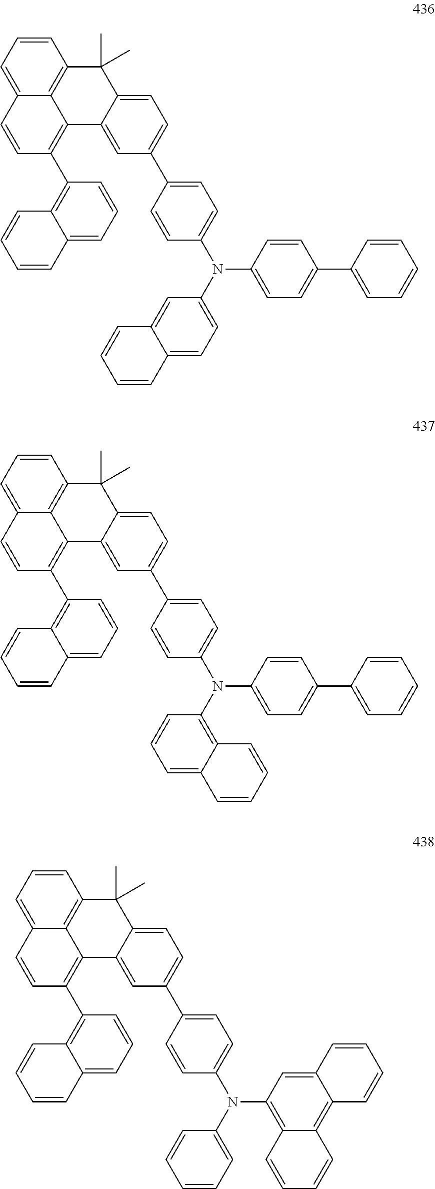 [SMILES: CC1(C)c2ccc(-c3ccc(N(c4ccc(-c5ccccc5)cc4)c4ccc5ccccc5c4)cc3)cc2-c2c(-c3cccc4ccccc34)ccc3cccc1c23 ; CC1(C)c2ccc(-c3ccc(N(c4ccc(-c5ccccc5)cc4)c4cccc5ccccc45)cc3)cc2-c2c(-c3cccc4ccccc34)ccc3cccc1c23 ; CC1(C)c2ccc(-c3ccc(N(c4ccccc4)c4cc5ccccc5c5ccccc45)cc3)cc2-c2c(-c3cccc4ccccc34)ccc3cccc1c23]

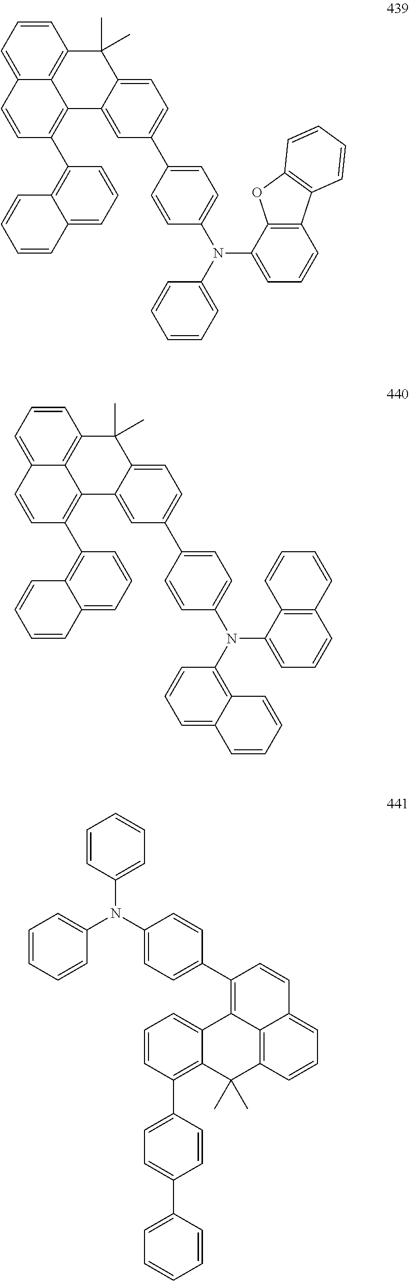 [SMILES: CC1(C)c2ccc(-c3ccc(N(c4ccccc4)c4cccc5c4oc4ccccc45)cc3)cc2-c2c(-c3cccc4ccccc34)ccc3cccc1c23 ; CC1(C)c2ccc(-c3ccc(N(c4cccc5ccccc45)c4cccc5ccccc45)cc3)cc2-c2c(-c3cccc4ccccc34)ccc3cccc1c23 ; CC1(C)c2c(-c3ccc(-c4ccccc4)cc3)cccc2-c2c(-c3ccc(N(c4ccccc4)c4ccccc4)cc3)ccc3cccc1c23]

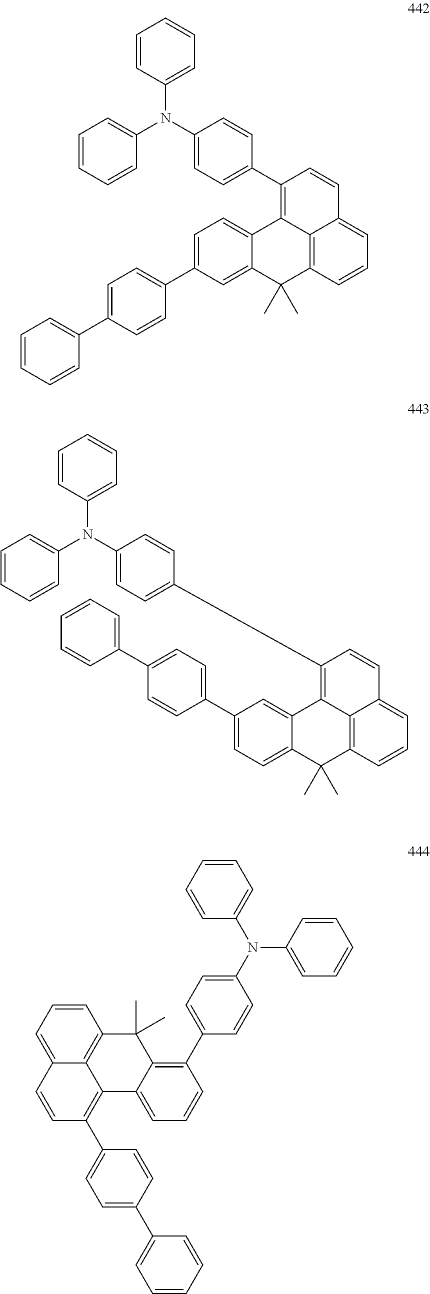 [SMILES: CC1(C)c2cc(-c3ccc(-c4ccccc4)cc3)ccc2-c2c(-c3ccc(N(c4ccccc4)c4ccccc4)cc3)ccc3cccc1c23 ; CC1(C)c2ccc(-c3ccc(-c4ccccc4)cc3)cc2-c2c(-c3ccc(N(c4ccccc4)c4ccccc4)cc3)ccc3cccc1c23 ; CC1(C)c2c(-c3ccc(N(c4ccccc4)c4ccccc4)cc3)cccc2-c2c(-c3ccc(-c4ccccc4)cc3)ccc3cccc1c23]

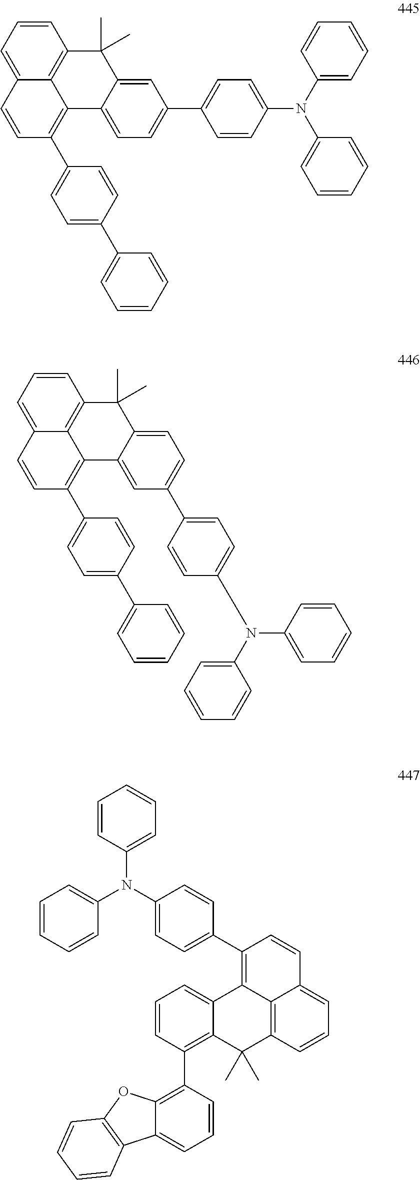 [SMILES: CC1(C)c2cc(-c3ccc(N(c4ccccc4)c4ccccc4)cc3)ccc2-c2c(-c3ccc(-c4ccccc4)cc3)ccc3cccc1c23 ; CC1(C)c2ccc(-c3ccc(N(c4ccccc4)c4ccccc4)cc3)cc2-c2c(-c3ccc(-c4ccccc4)cc3)ccc3cccc1c23 ; CC1(C)c2c(-c3cccc4c3oc3ccccc34)cccc2-c2c(-c3ccc(N(c4ccccc4)c4ccccc4)cc3)ccc3cccc1c23]

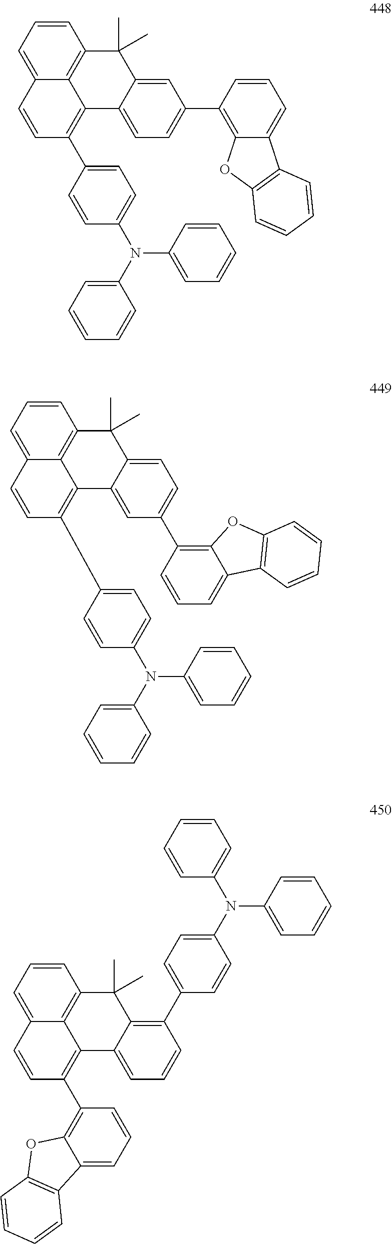 [SMILES: CC1(C)c2cc(-c3cccc4c3oc3ccccc34)ccc2-c2c(-c3ccc(N(c4ccccc4)c4ccccc4)cc3)ccc3cccc1c23 ; CC1(C)c2ccc(-c3cccc4c3oc3ccccc34)cc2-c2c(-c3ccc(N(c4ccccc4)c4ccccc4)cc3)ccc3cccc1c23 ; CC1(C)c2c(-c3ccc(N(c4ccccc4)c4ccccc4)cc3)cccc2-c2c(-c3cccc4c3oc3ccccc34)ccc3cccc1c23]

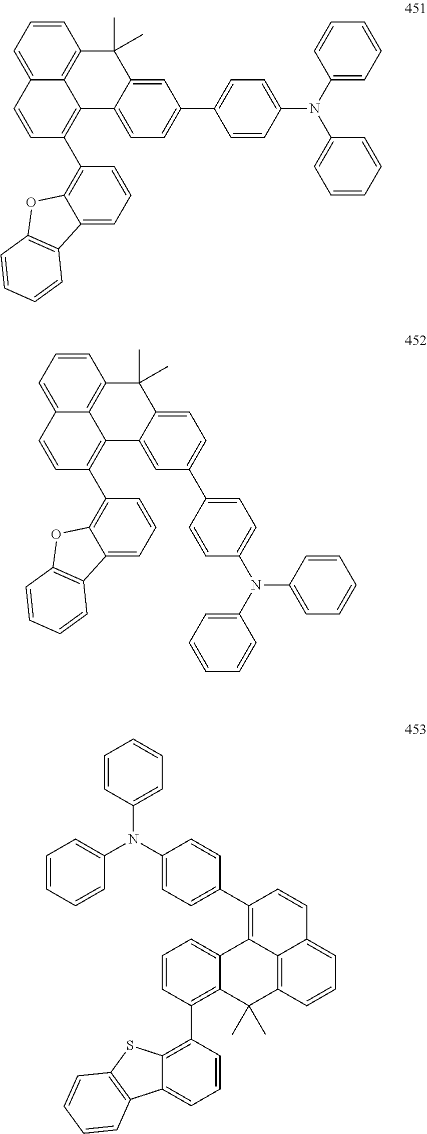 [SMILES: CC1(C)c2cc(-c3ccc(N(c4ccccc4)c4ccccc4)cc3)ccc2-c2c(-c3cccc4c3oc3ccccc34)ccc3cccc1c23 ; CC1(C)c2ccc(-c3ccc(N(c4ccccc4)c4ccccc4)cc3)cc2-c2c(-c3cccc4c3oc3ccccc34)ccc3cccc1c23 ; CC1(C)c2c(-c3cccc4c3sc3ccccc34)cccc2-c2c(-c3ccc(N(c4ccccc4)c4ccccc4)cc3)ccc3cccc1c23]

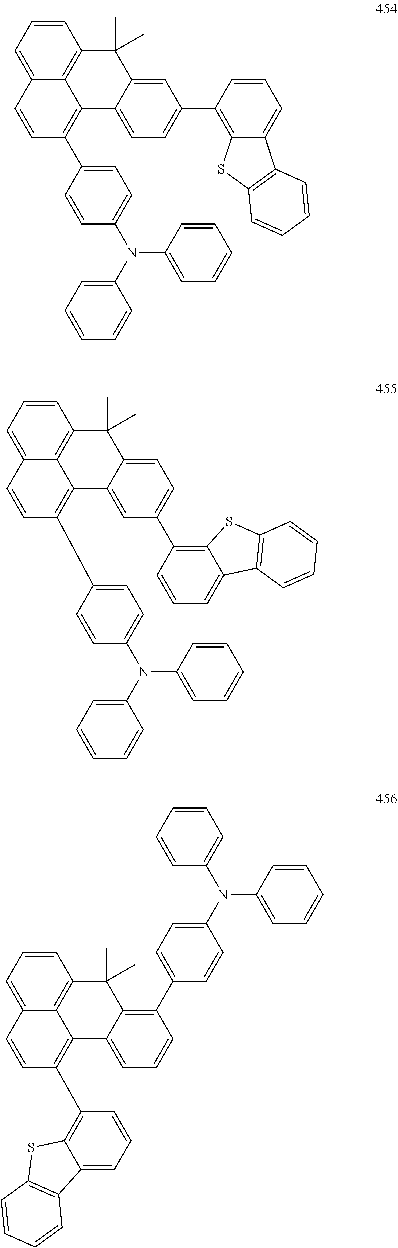 [SMILES: CC1(C)c2cc(-c3cccc4c3sc3ccccc34)ccc2-c2c(-c3ccc(N(c4ccccc4)c4ccccc4)cc3)ccc3cccc1c23 ; CC1(C)c2ccc(-c3cccc4c3sc3ccccc34)cc2-c2c(-c3ccc(N(c4ccccc4)c4ccccc4)cc3)ccc3cccc1c23 ; CC1(C)c2c(-c3ccc(N(c4ccccc4)c4ccccc4)cc3)cccc2-c2c(-c3cccc4c3sc3ccccc34)ccc3cccc1c23]

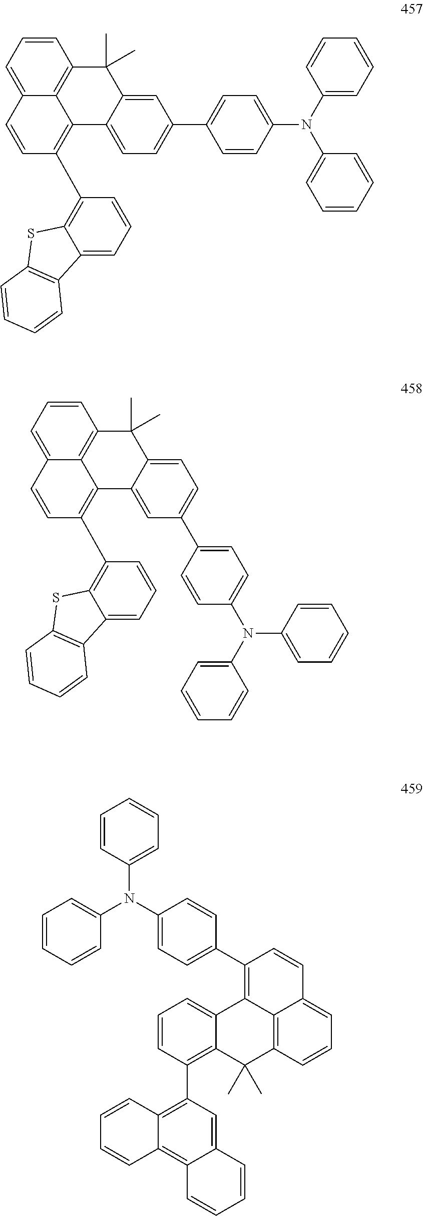 [SMILES: CC1(C)c2cc(-c3ccc(N(c4ccccc4)c4ccccc4)cc3)ccc2-c2c(-c3cccc4c3sc3ccccc34)ccc3cccc1c23 ; CC1(C)c2ccc(-c3ccc(N(c4ccccc4)c4ccccc4)cc3)cc2-c2c(-c3cccc4c3sc3ccccc34)ccc3cccc1c23 ; CC1(C)c2c(-c3cc4ccccc4c4ccccc34)cccc2-c2c(-c3ccc(N(c4ccccc4)c4ccccc4)cc3)ccc3cccc1c23]

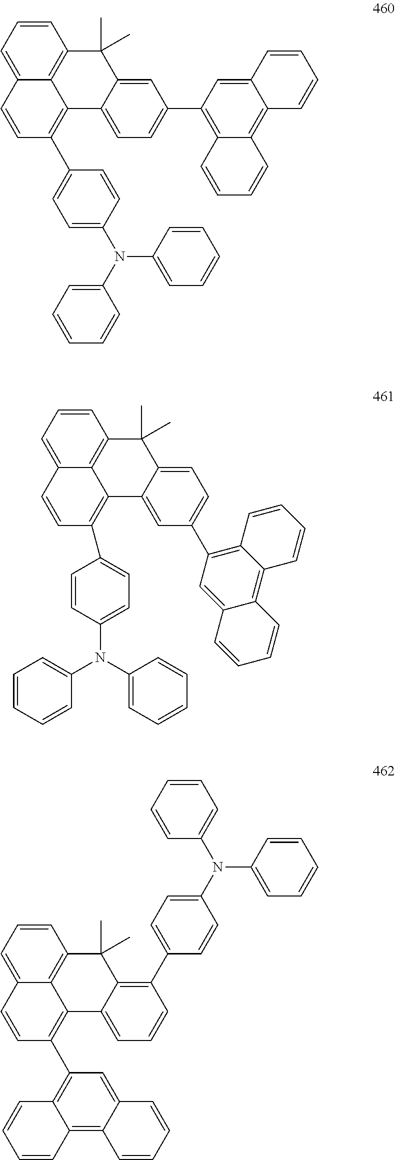 [SMILES: CC1(C)c2cc(-c3cc4ccccc4c4ccccc34)ccc2-c2c(-c3ccc(N(c4ccccc4)c4ccccc4)cc3)ccc3cccc1c23 ; CC1(C)c2ccc(-c3cc4ccccc4c4ccccc34)cc2-c2c(-c3ccc(N(c4ccccc4)c4ccccc4)cc3)ccc3cccc1c23 ; CC1(C)c2c(-c3ccc(N(c4ccccc4)c4ccccc4)cc3)cccc2-c2c(-c3cc4ccccc4c4ccccc34)ccc3cccc1c23]

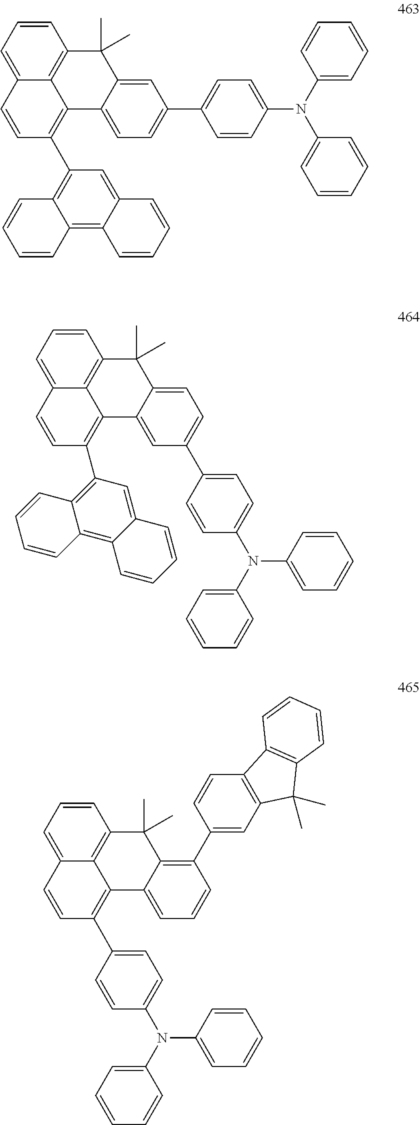 [SMILES: CC1(C)c2cc(-c3ccc(N(c4ccccc4)c4ccccc4)cc3)ccc2-c2c(-c3cc4ccccc4c4ccccc34)ccc3cccc1c23 ; CC1(C)c2ccc(-c3ccc(N(c4ccccc4)c4ccccc4)cc3)cc2-c2c(-c3cc4ccccc4c4ccccc34)ccc3cccc1c23 ; CC1(C)c2ccccc2-c2ccc(-c3cccc4c3C(C)(C)c3cccc5ccc(-c6ccc(N(c7ccccc7)c7ccccc7)cc6)c-4c35)cc21]

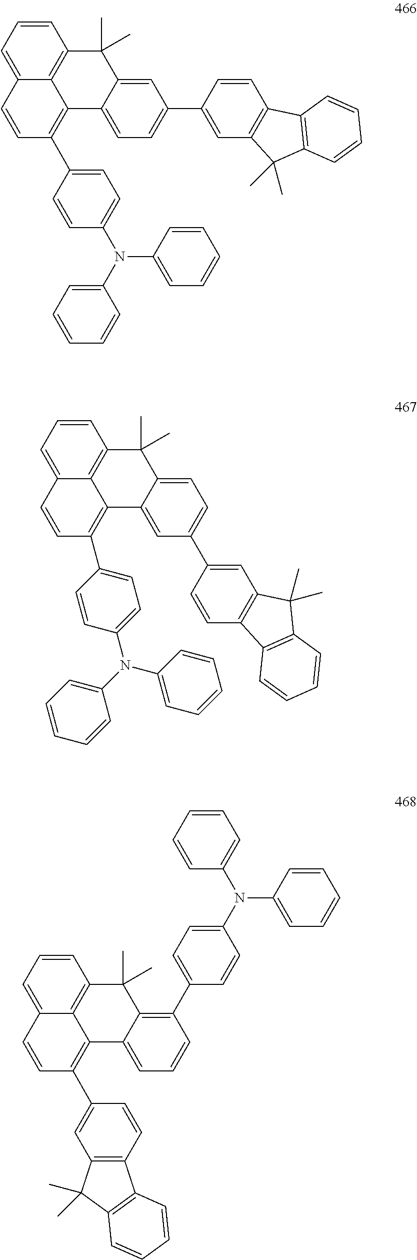 [SMILES: CC1(C)c2ccccc2-c2ccc(-c3ccc4c(c3)C(C)(C)c3cccc5ccc(-c6ccc(N(c7ccccc7)c7ccccc7)cc6)c-4c35)cc21 ; CC1(C)c2ccccc2-c2ccc(-c3ccc4c(c3)-c3c(-c5ccc(N(c6ccccc6)c6ccccc6)cc5)ccc5cccc(c35)C4(C)C)cc21 ; CC1(C)c2ccccc2-c2ccc(-c3ccc4cccc5c4c3-c3cccc(-c4ccc(N(c6ccccc6)c6ccccc6)cc4)c3C5(C)C)cc21]

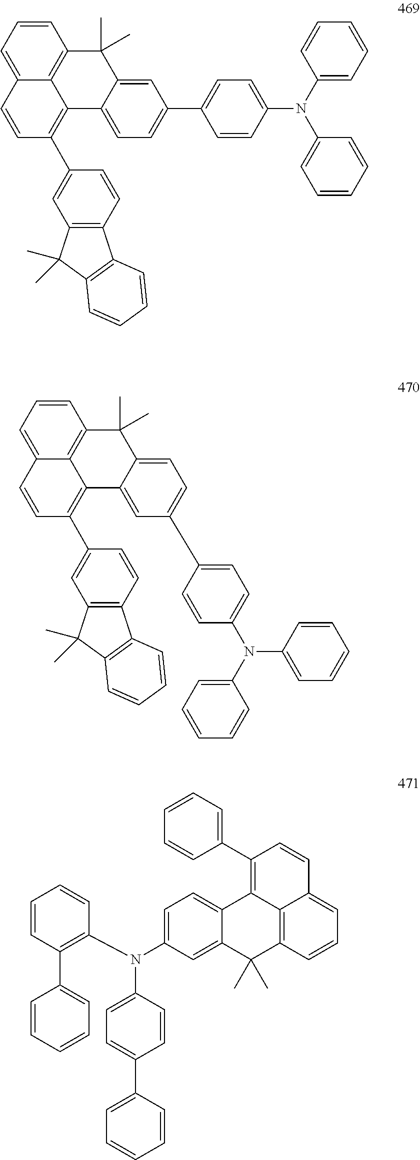 [SMILES: CC1(C)c2ccccc2-c2ccc(-c3ccc4cccc5c4c3-c3ccc(-c4ccc(N(c6ccccc6)c6ccccc6)cc4)cc3C5(C)C)cc21 ; CC1(C)c2ccccc2-c2ccc(-c3ccc4cccc5c4c3-c3cc(-c4ccc(N(c6ccccc6)c6ccccc6)cc4)ccc3C5(C)C)cc21 ; CC1(C)c2cc(N(c3ccc(-c4ccccc4)cc3)c3ccccc3-c3ccccc3)ccc2-c2c(-c3ccccc3)ccc3cccc1c23]

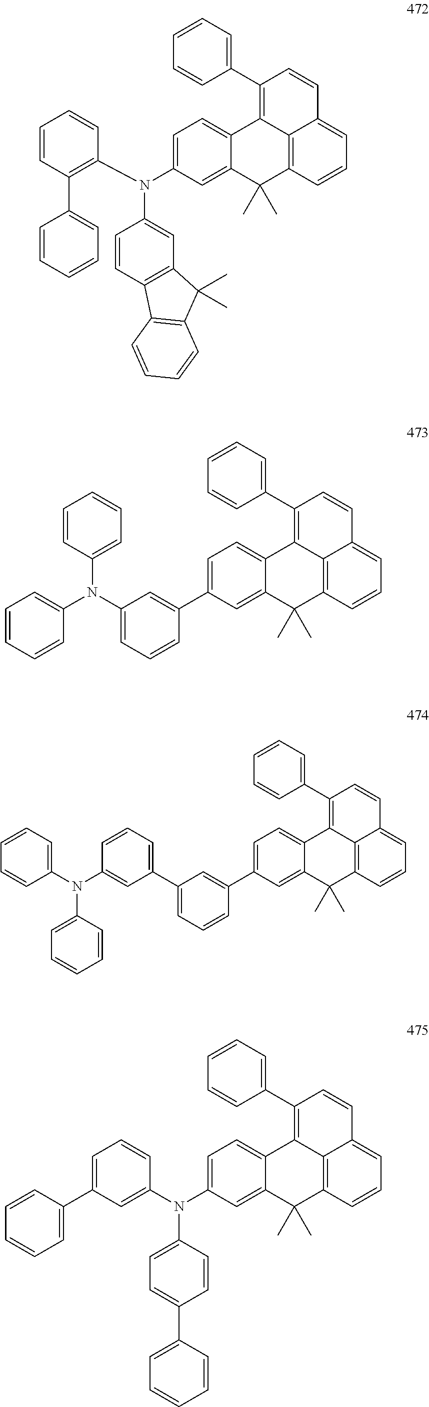 [SMILES: CC1(C)c2ccccc2-c2ccc(N(c3ccc4c(c3)C(C)(C)c3cccc5ccc(-c6ccccc6)c-4c35)c3ccccc3-c3ccccc3)cc21 ; CC1(C)c2cc(-c3cccc(N(c4ccccc4)c4ccccc4)c3)ccc2-c2c(-c3ccccc3)ccc3cccc1c23 ; CC1(C)c2cc(-c3cccc(-c4cccc(N(c5ccccc5)c5ccccc5)c4)c3)ccc2-c2c(-c3ccccc3)ccc3cccc1c23 ; CC1(C)c2cc(N(c3ccc(-c4ccccc4)cc3)c3cccc(-c4ccccc4)c3)ccc2-c2c(-c3ccccc3)ccc3cccc1c23]

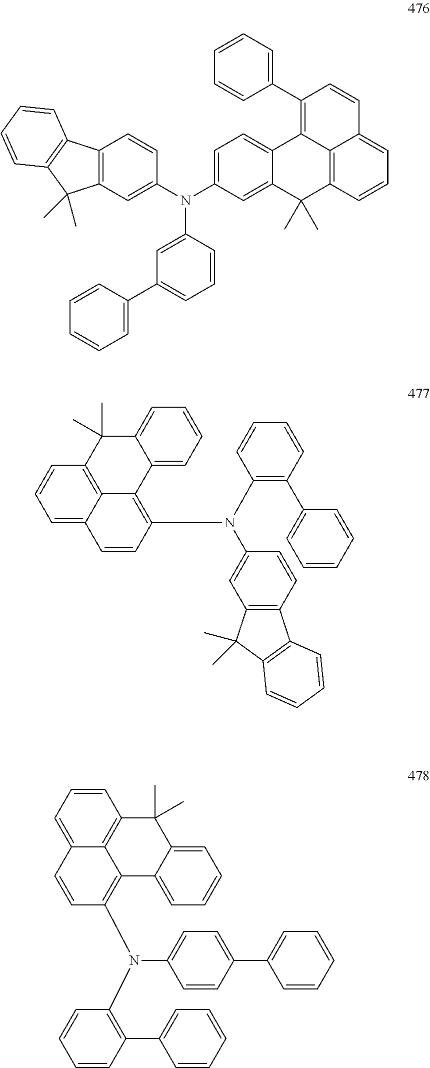 [SMILES: CC1(C)c2ccccc2-c2ccc(N(c3cccc(-c4ccccc4)c3)c3ccc4c(c3)C(C)(C)c3cccc5ccc(-c6ccccc6)c-4c35)cc21 ; CC1(C)c2ccccc2-c2ccc(N(c3ccccc3-c3ccccc3)c3ccc4cccc5c4c3-c3ccccc3C5(C)C)cc21 ; CC1(C)c2ccccc2-c2c(N(c3ccc(-c4ccccc4)cc3)c3ccccc3-c3ccccc3)ccc3cccc1c23]

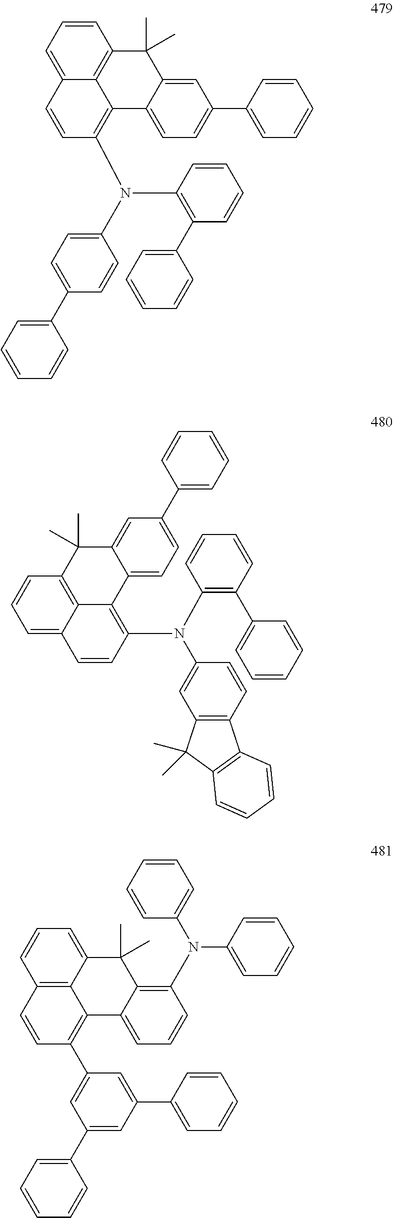 [SMILES: CC1(C)c2cc(-c3ccccc3)ccc2-c2c(N(c3ccc(-c4ccccc4)cc3)c3ccccc3-c3ccccc3)ccc3cccc1c23 ; CC1(C)c2ccccc2-c2ccc(N(c3ccccc3-c3ccccc3)c3ccc4cccc5c4c3-c3ccc(-c4ccccc4)cc3C5(C)C)cc21 ; CC1(C)c2c(cccc2N(c2ccccc2)c2ccccc2)-c2c(-c3cc(-c4ccccc4)cc(-c4ccccc4)c3)ccc3cccc1c23]

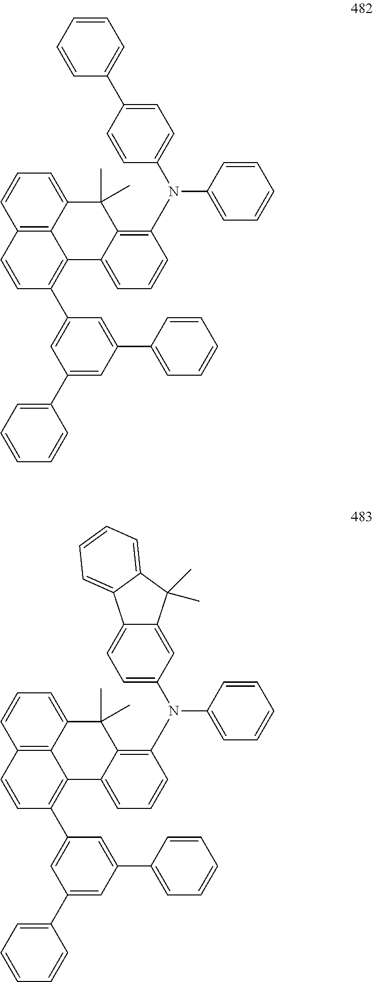 [SMILES: CC1(C)c2c(cccc2N(c2ccccc2)c2ccc(-c3ccccc3)cc2)-c2c(-c3cc(-c4ccccc4)cc(-c4ccccc4)c3)ccc3cccc1c23 ; CC1(C)c2ccccc2-c2ccc(N(c3ccccc3)c3cccc4c3C(C)(C)c3cccc5ccc(-c6cc(-c7ccccc7)cc(-c7ccccc7)c6)c-4c35)cc21]

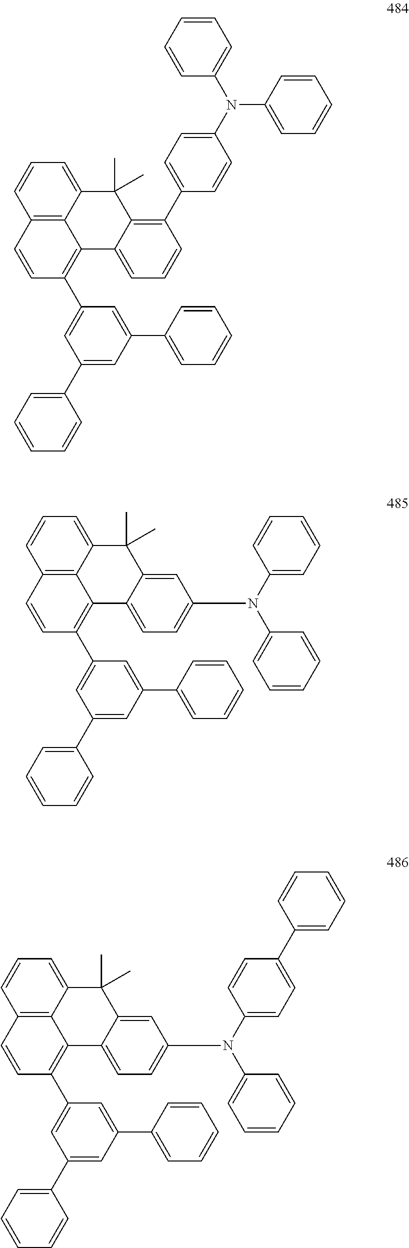 [SMILES: CC1(C)c2c(-c3ccc(N(c4ccccc4)c4ccccc4)cc3)cccc2-c2c(-c3cc(-c4ccccc4)cc(-c4ccccc4)c3)ccc3cccc1c23 ; CC1(C)c2cc(N(c3ccccc3)c3ccccc3)ccc2-c2c(-c3cc(-c4ccccc4)cc(-c4ccccc4)c3)ccc3cccc1c23 ; CC1(C)c2cc(N(c3ccccc3)c3ccc(-c4ccccc4)cc3)ccc2-c2c(-c3cc(-c4ccccc4)cc(-c4ccccc4)c3)ccc3cccc1c23]

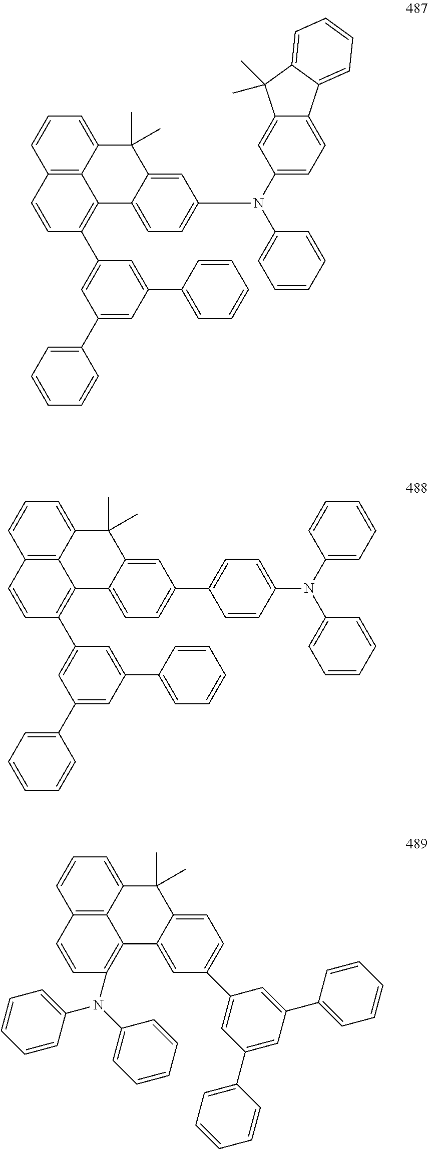 [SMILES: CC1(C)c2ccccc2-c2ccc(N(c3ccccc3)c3ccc4c(c3)C(C)(C)c3cccc5ccc(-c6cc(-c7ccccc7)cc(-c7ccccc7)c6)c-4c35)cc21 ; CC1(C)c2cc(-c3ccc(N(c4ccccc4)c4ccccc4)cc3)ccc2-c2c(-c3cc(-c4ccccc4)cc(-c4ccccc4)c3)ccc3cccc1c23 ; CC1(C)c2ccc(-c3cc(-c4ccccc4)cc(-c4ccccc4)c3)cc2-c2c(N(c3ccccc3)c3ccccc3)ccc3cccc1c23]

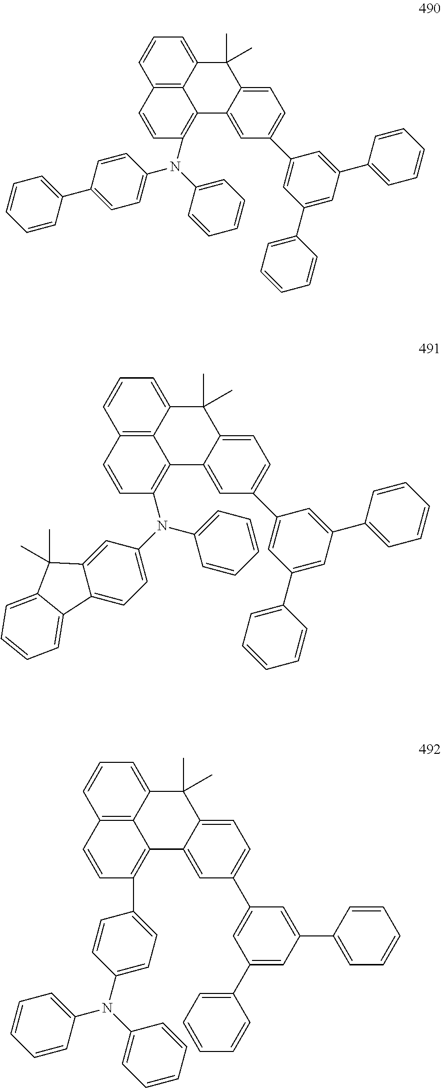 [SMILES: CC1(C)c2ccc(-c3cc(-c4ccccc4)cc(-c4ccccc4)c3)cc2-c2c(N(c3ccccc3)c3ccc(-c4ccccc4)cc3)ccc3cccc1c23 ; CC1(C)c2ccccc2-c2ccc(N(c3ccccc3)c3ccc4cccc5c4c3-c3cc(-c4cc(-c6ccccc6)cc(-c6ccccc6)c4)ccc3C5(C)C)cc21 ; CC1(C)c2ccc(-c3cc(-c4ccccc4)cc(-c4ccccc4)c3)cc2-c2c(-c3ccc(N(c4ccccc4)c4ccccc4)cc3)ccc3cccc1c23]

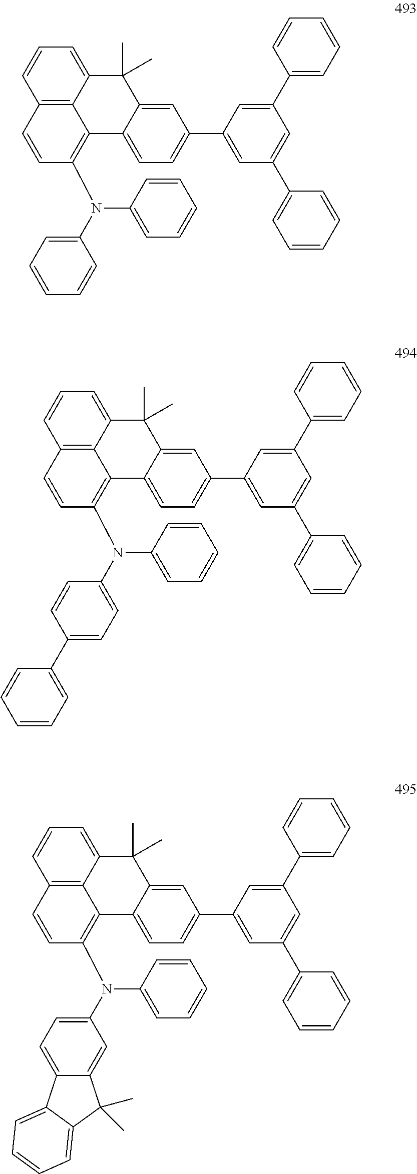 [SMILES: CC1(C)c2cc(-c3cc(-c4ccccc4)cc(-c4ccccc4)c3)ccc2-c2c(N(c3ccccc3)c3ccccc3)ccc3cccc1c23 ; CC1(C)c2cc(-c3cc(-c4ccccc4)cc(-c4ccccc4)c3)ccc2-c2c(N(c3ccccc3)c3ccc(-c4ccccc4)cc3)ccc3cccc1c23 ; CC1(C)c2ccccc2-c2ccc(N(c3ccccc3)c3ccc4cccc5c4c3-c3ccc(-c4cc(-c6ccccc6)cc(-c6ccccc6)c4)cc3C5(C)C)cc21]

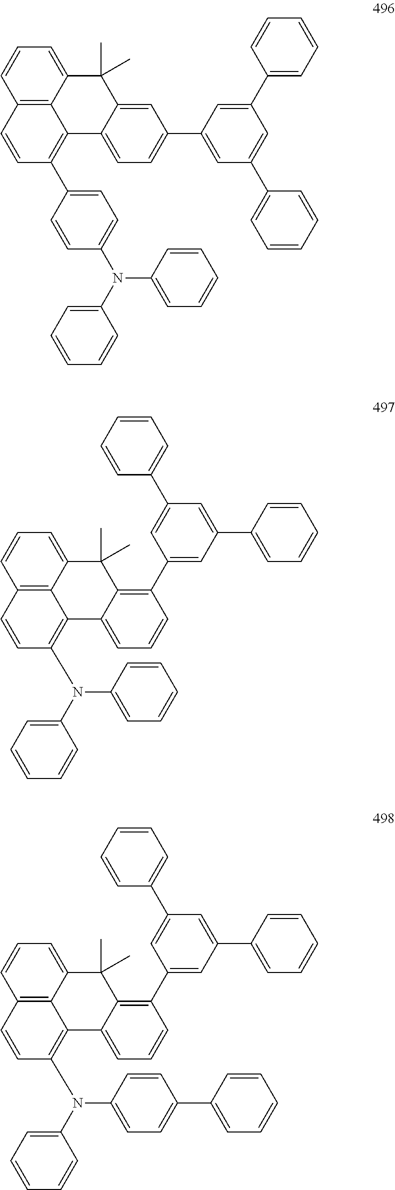 [SMILES: CC1(C)c2cc(-c3cc(-c4ccccc4)cc(-c4ccccc4)c3)ccc2-c2c(-c3ccc(N(c4ccccc4)c4ccccc4)cc3)ccc3cccc1c23 ; CC1(C)c2c(-c3cc(-c4ccccc4)cc(-c4ccccc4)c3)cccc2-c2c(N(c3ccccc3)c3ccccc3)ccc3cccc1c23 ; CC1(C)c2c(-c3cc(-c4ccccc4)cc(-c4ccccc4)c3)cccc2-c2c(N(c3ccccc3)c3ccc(-c4ccccc4)cc3)ccc3cccc1c23]

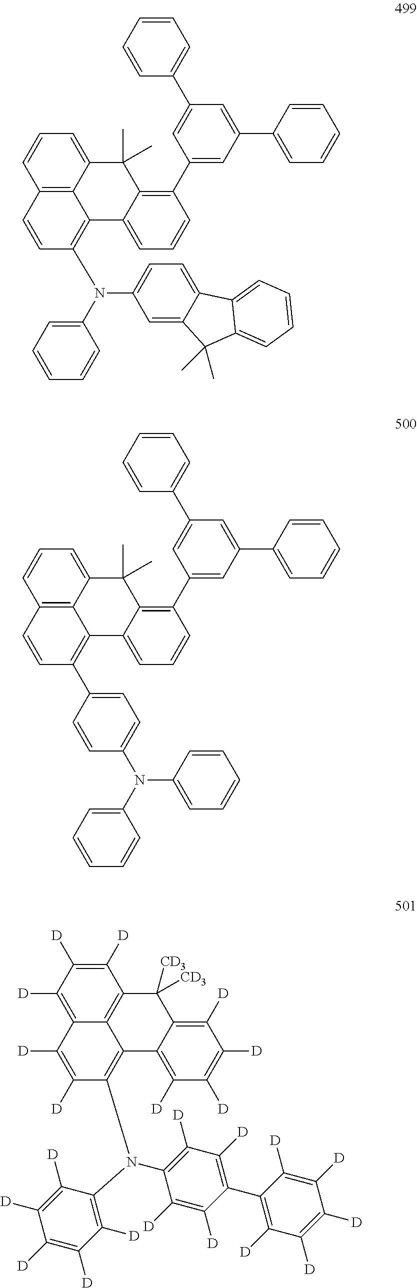 [SMILES: CC1(C)c2ccccc2-c2ccc(N(c3ccccc3)c3ccc4cccc5c4c3-c3cccc(-c4cc(-c6ccccc6)cc(-c6ccccc6)c4)c3C5(C)C)cc21 ; CC1(C)c2c(-c3cc(-c4ccccc4)cc(-c4ccccc4)c3)cccc2-c2c(-c3ccc(N(c4ccccc4)c4ccccc4)cc3)ccc3cccc1c23 ; [2H]c1c([2H])c([2H])c(-c2c([2H])c([2H])c(N(c3c([2H])c([2H])c([2H])c([2H])c3[2H])c3c([2H])c([2H])c4c([2H])c([2H])c([2H])c5c4c3-c3c([2H])c([2H])c([2H])c([2H])c3C5(C([2H])([2H])[2H])C([2H])([2H])[2H])c([2H])c2[2H])c([2H])c1[2H]]

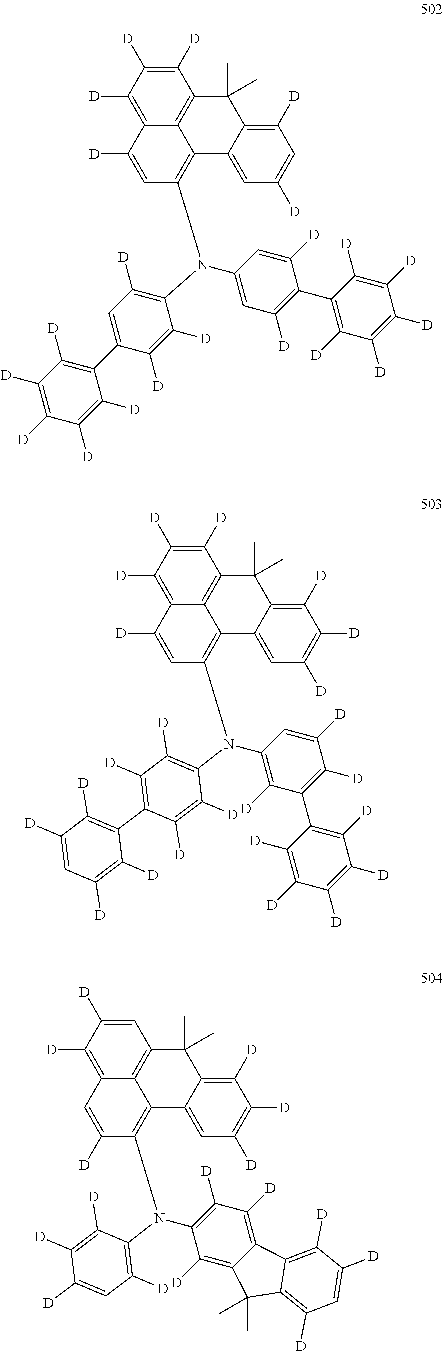

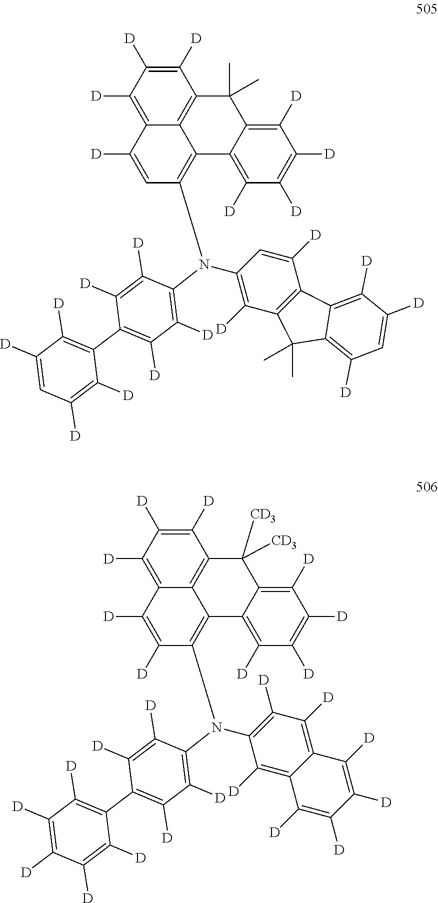 [SMILES: [2H]c1cc([2H])c([2H])c(-c2c([2H])c([2H])c(N(c3cc([2H])c4c(c3[2H])C(C)(C)c3c([2H])cc([2H])c([2H])c3-4)c3cc([2H])c4c([2H])c([2H])c([2H])c5c4c3-c3c([2H])c([2H])c([2H])c([2H])c3C5(C)C)c([2H])c2[2H])c1[2H] ; [2H]c1c([2H])c([2H])c(-c2c([2H])c([2H])c(N(c3c([2H])c([2H])c4c([2H])c([2H])c([2H])c([2H])c4c3[2H])c3c([2H])c([2H])c4c([2H])c([2H])c([2H])c5c4c3-c3c([2H])c([2H])c([2H])c([2H])c3C5(C([2H])([2H])[2H])C([2H])([2H])[2H])c([2H])c2[2H])c([2H])c1[2H]]

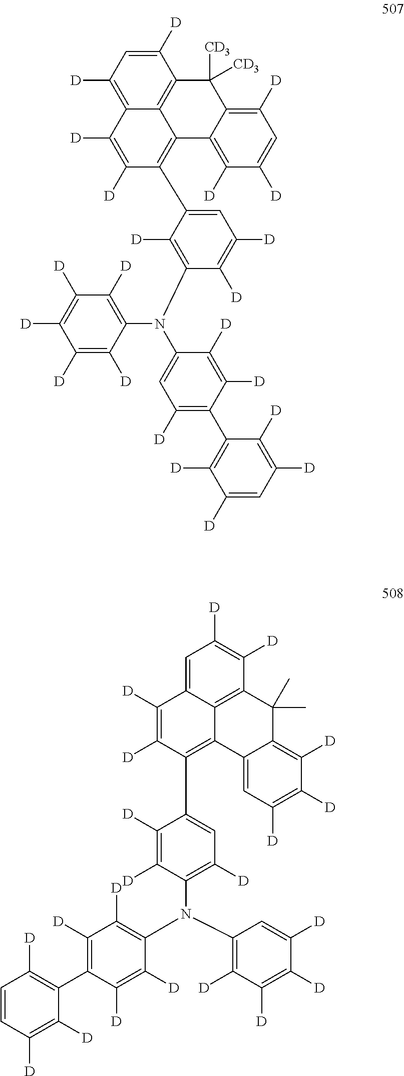 [SMILES: [2H]c1cc([2H])c([2H])c(-c2c([2H])cc(N(c3c([2H])c([2H])cc(-c4c([2H])c([2H])c5c([2H])cc([2H])c6c5c4-c4c([2H])c([2H])cc([2H])c4C6(C([2H])([2H])[2H])C([2H])([2H])[2H])c3[2H])c3c([2H])c([2H])c([2H])c([2H])c3[2H])c([2H])c2[2H])c1[2H] ; [2H]c1cc(N(c2c([2H])cc(-c3c([2H])c([2H])c4cc([2H])c([2H])c5c4c3-c3cc([2H])c([2H])c([2H])c3C5(C)C)c([2H])c2[2H])c2c([2H])c([2H])c(-c3c([2H])ccc([2H])c3[2H])c([2H])c2[2H])c([2H])c([2H])c1[2H]]

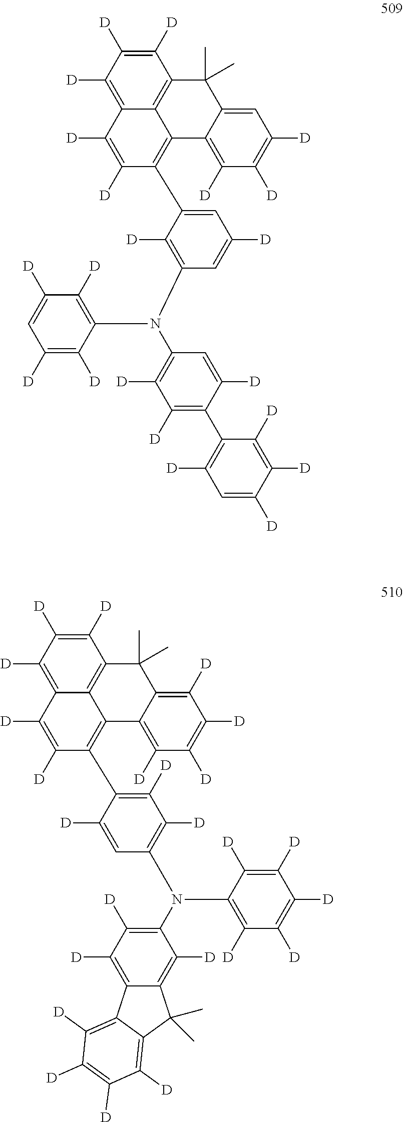 [SMILES: [2H]c1cc(-c2c([2H])c([2H])c3c([2H])c([2H])c([2H])c4c3c2-c2c(cc([2H])c([2H])c2[2H])C4(C)C)c([2H])c(N(c2cc([2H])c(-c3c([2H])cc([2H])c([2H])c3[2H])c([2H])c2[2H])c2c([2H])c([2H])cc([2H])c2[2H])c1 ; [2H]c1cc(N(c2c([2H])c([2H])c([2H])c([2H])c2[2H])c2c([2H])c([2H])c3c(c2[2H])C(C)(C)c2c([2H])c([2H])c([2H])c([2H])c2-3)c([2H])c([2H])c1-c1c([2H])c([2H])c2c([2H])c([2H])c([2H])c3c2c1-c1c([2H])c([2H])c([2H])c([2H])c1C3(C)C]

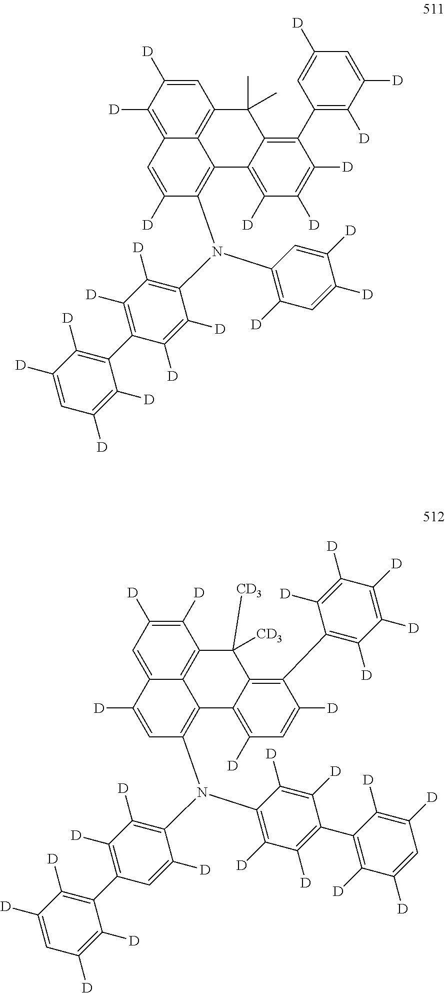 [SMILES: [2H]c1cc([2H])c([2H])c(-c2c([2H])c([2H])c([2H])c3c2C(C)(C)c2cc([2H])c([2H])c4cc([2H])c(N(c5cc([2H])c([2H])cc5[2H])c5c([2H])c([2H])c(-c6c([2H])c([2H])cc([2H])c6[2H])c([2H])c5[2H])c-3c24)c1 ; [2H]c1cc([2H])c([2H])c(-c2cc([2H])c(N(c3cc([2H])c4cc([2H])c([2H])c5c4c3-c3c([2H])cc([2H])c(-c4c([2H])c([2H])c([2H])c([2H])c4[2H])c3C5(C([2H])([2H])[2H])C([2H])([2H])[2H])c3c([2H])c([2H])c(-c4c([2H])c([2H])cc([2H])c4[2H])c([2H])c3[2H])c([2H])c2[2H])c1[2H]]

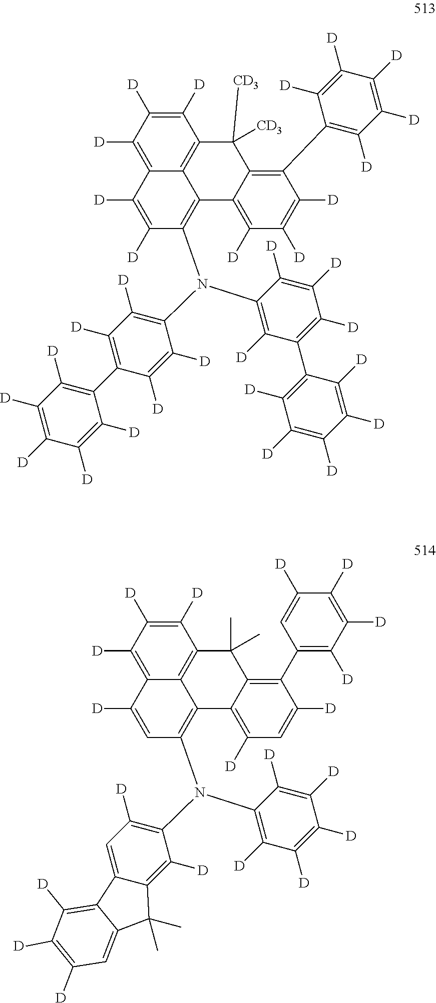 [SMILES: [2H]c1c([2H])c([2H])c(-c2c([2H])c([2H])c(N(c3c([2H])c([2H])c([2H])c(-c4c([2H])c([2H])c([2H])c([2H])c4[2H])c3[2H])c3c([2H])c([2H])c4c([2H])c([2H])c([2H])c5c4c3-c3c([2H])c([2H])c([2H])c(-c4c([2H])c([2H])c([2H])c([2H])c4[2H])c3C5(C([2H])([2H])[2H])C([2H])([2H])[2H])c([2H])c2[2H])c([2H])c1[2H] ; [2H]c1cc(-c2c([2H])cc([2H])c3c2C(C)(C)c2c([2H])c([2H])c([2H])c4c([2H])cc(N(c5c([2H])cc6c(c5[2H])C(C)(C)c5cc([2H])c([2H])c([2H])c5-6)c5c([2H])c([2H])c([2H])c([2H])c5[2H])c-3c24)c([2H])c([2H])c1[2H]]

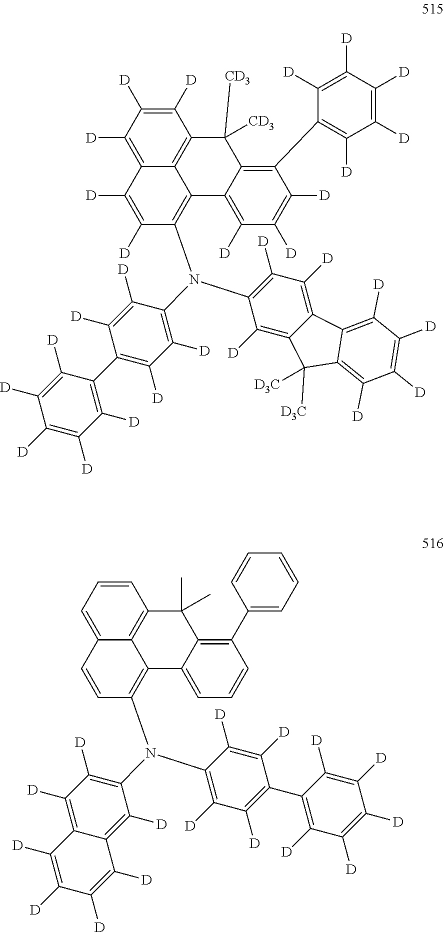 [SMILES: [2H]c1c([2H])c([2H])c(-c2c([2H])c([2H])c(N(c3c([2H])c([2H])c4c(c3[2H])C(C([2H])([2H])[2H])(C([2H])([2H])[2H])c3c([2H])c([2H])c([2H])c([2H])c3-4)c3c([2H])c([2H])c4c([2H])c([2H])c([2H])c5c4c3-c3c([2H])c([2H])c([2H])c(-c4c([2H])c([2H])c([2H])c([2H])c4[2H])c3C5(C([2H])([2H])[2H])C([2H])([2H])[2H])c([2H])c2[2H])c([2H])c1[2H] ; [2H]c1c([2H])c([2H])c(-c2c([2H])c([2H])c(N(c3ccc4cccc5c4c3-c3cccc(-c4ccccc4)c3C5(C)C)c3c([2H])c([2H])c4c([2H])c([2H])c([2H])c([2H])c4c3[2H])c([2H])c2[2H])c([2H])c1[2H]]

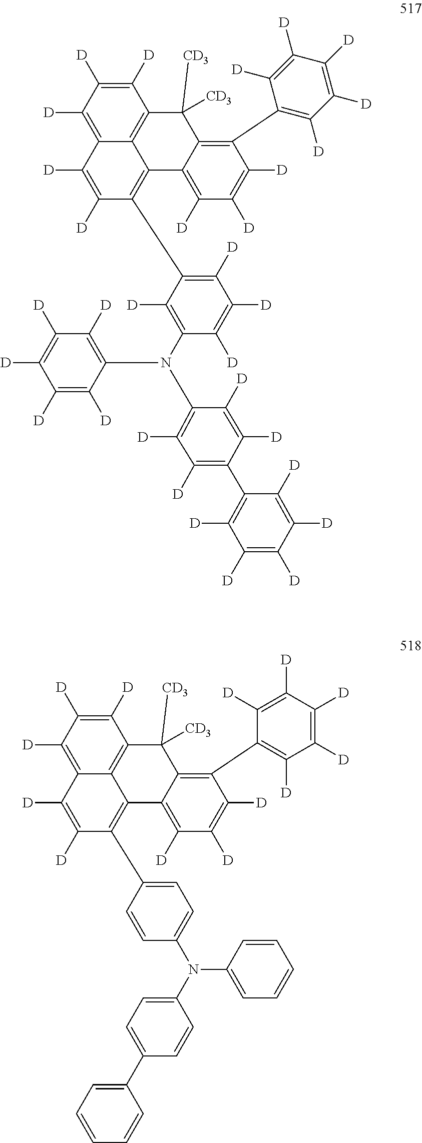 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4c([2H])c([2H])c5c([2H])c([2H])c([2H])c6c5c4-c4c([2H])c([2H])c([2H])c(-c5c([2H])c([2H])c([2H])c([2H])c5[2H])c4C6(C([2H])([2H])[2H])C([2H])([2H])[2H])c3[2H])c([2H])c2[2H])c([2H])c1[2H] ; [2H]c1c([2H])c([2H])c(-c2c([2H])c([2H])c([2H])c3c2C(C([2H])([2H])[2H])(C([2H])([2H])[2H])c2c([2H])c([2H])c([2H])c4c([2H])c([2H])c(-c5ccc(N(c6ccccc6)c6ccc(-c7ccccc7)cc6)cc5)c-3c24)c([2H])c1[2H]]

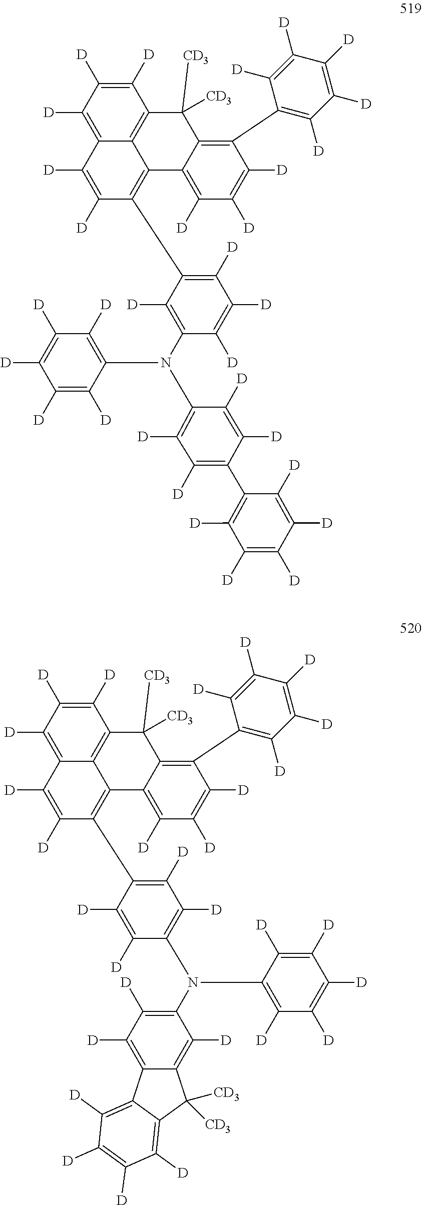

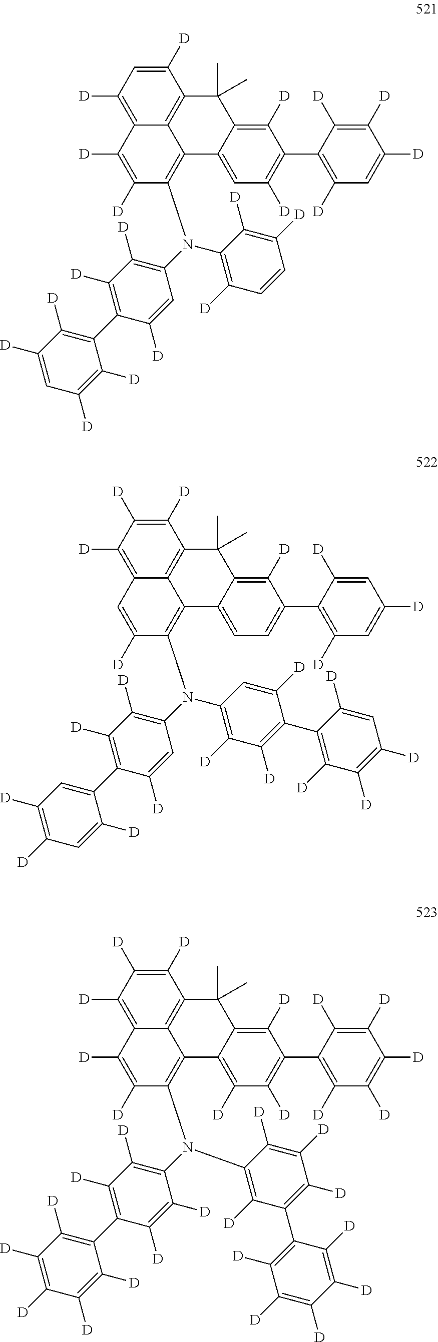

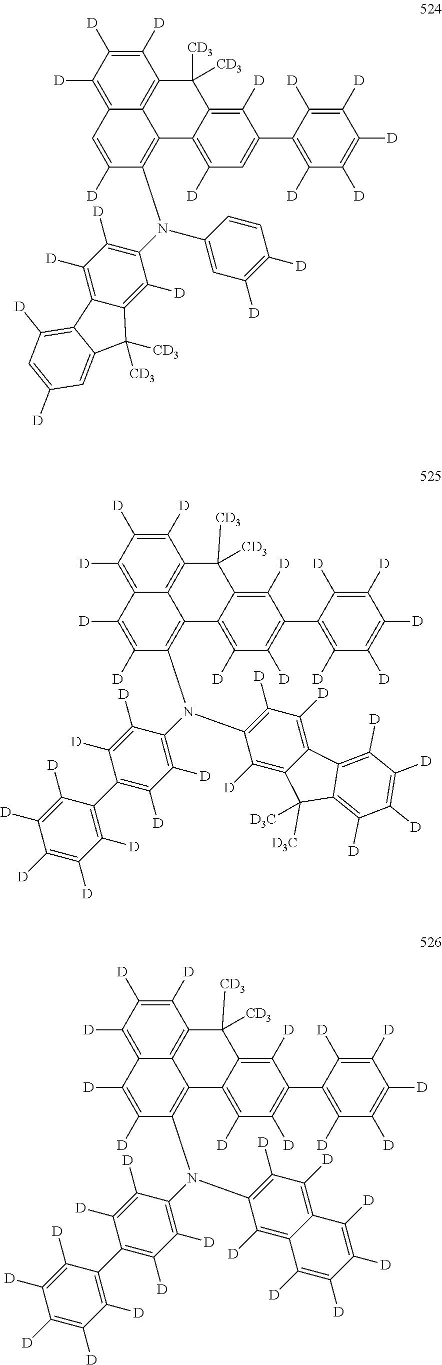

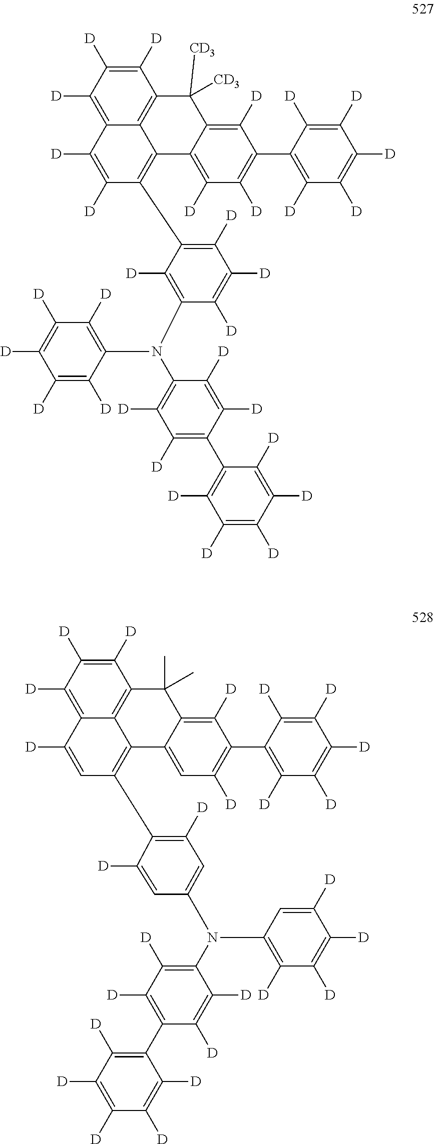

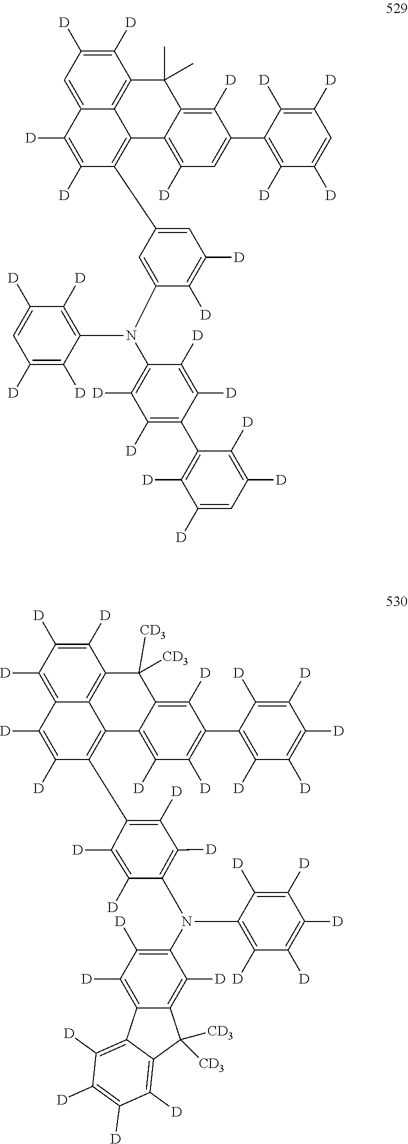

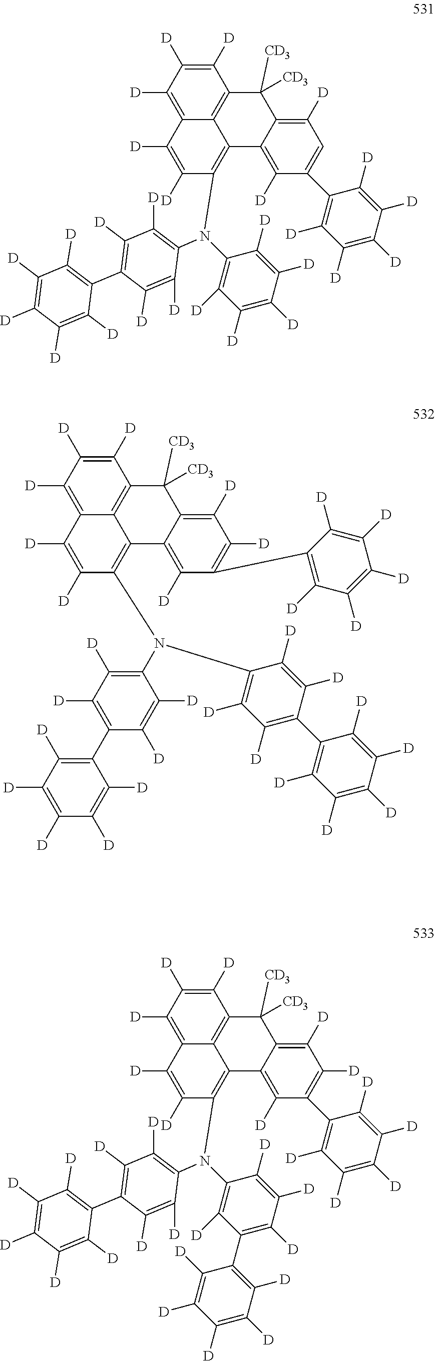

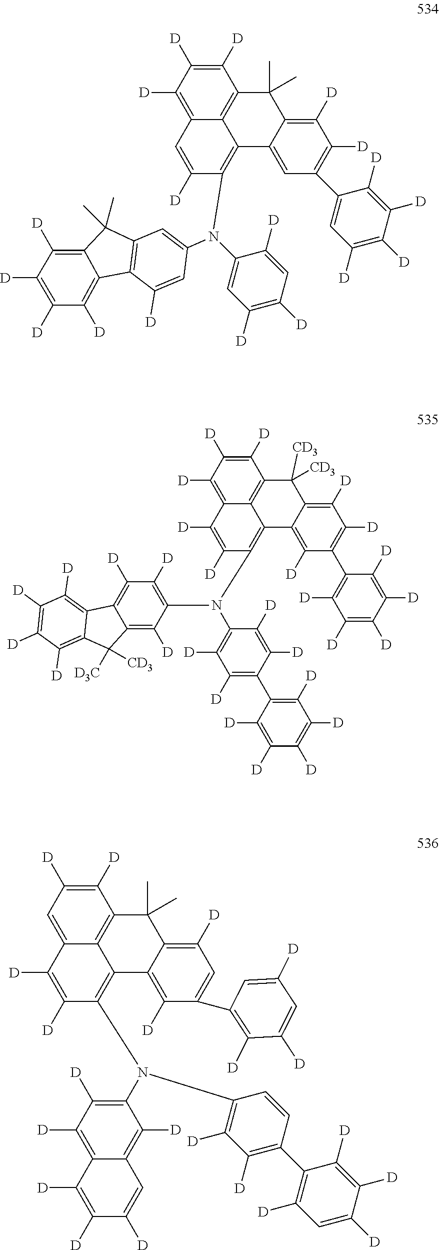

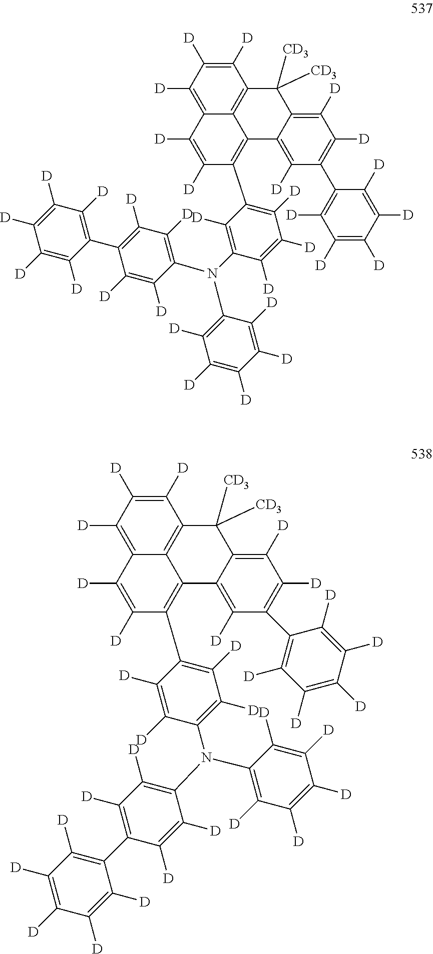

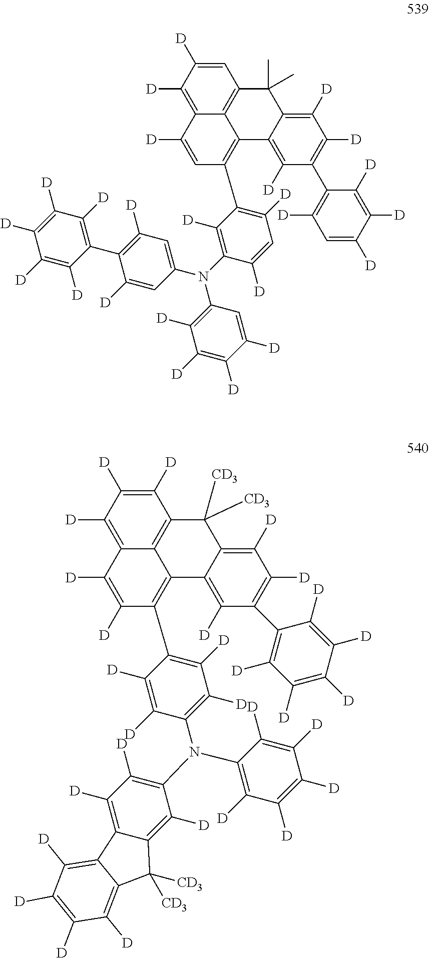

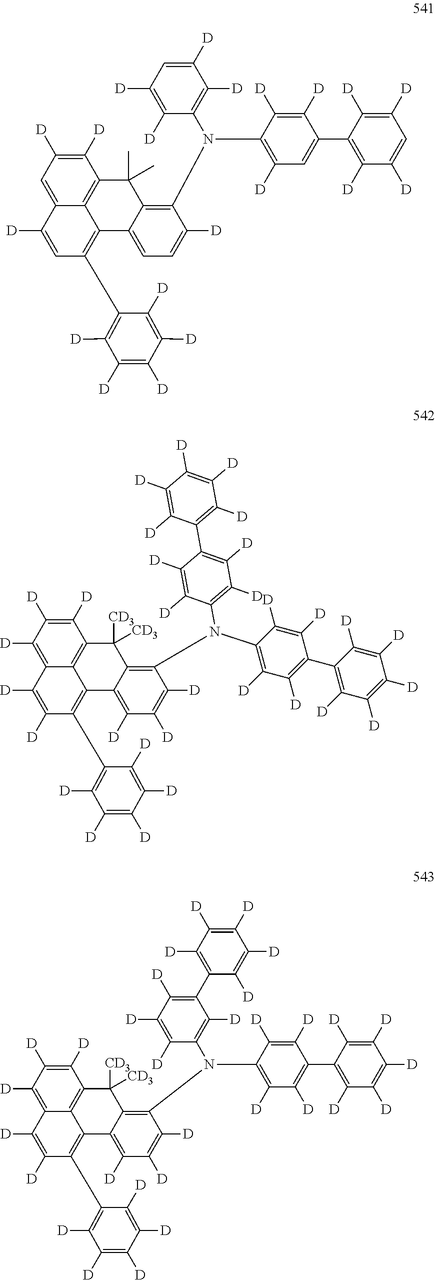

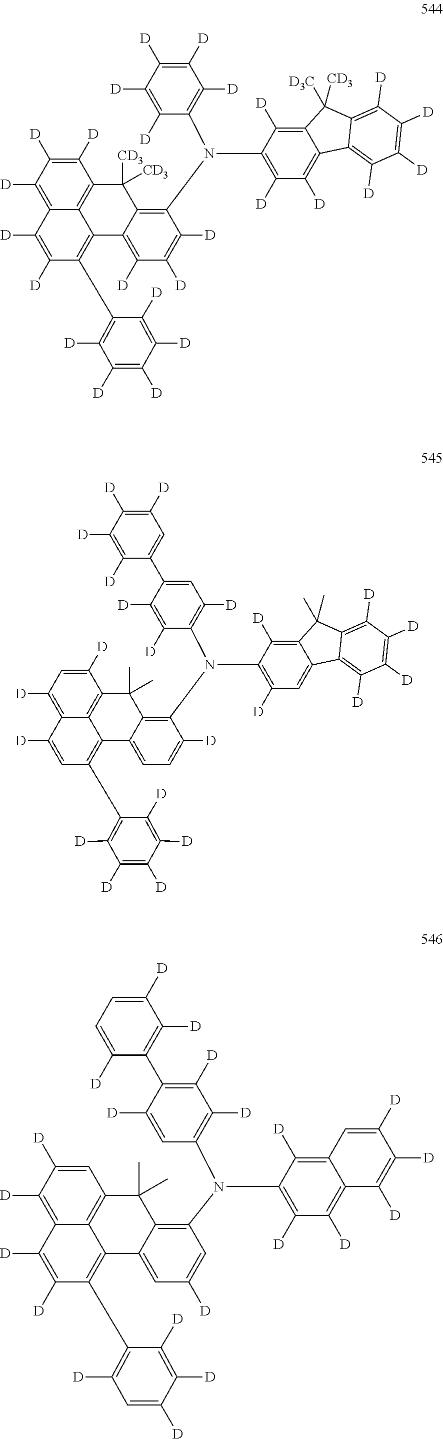

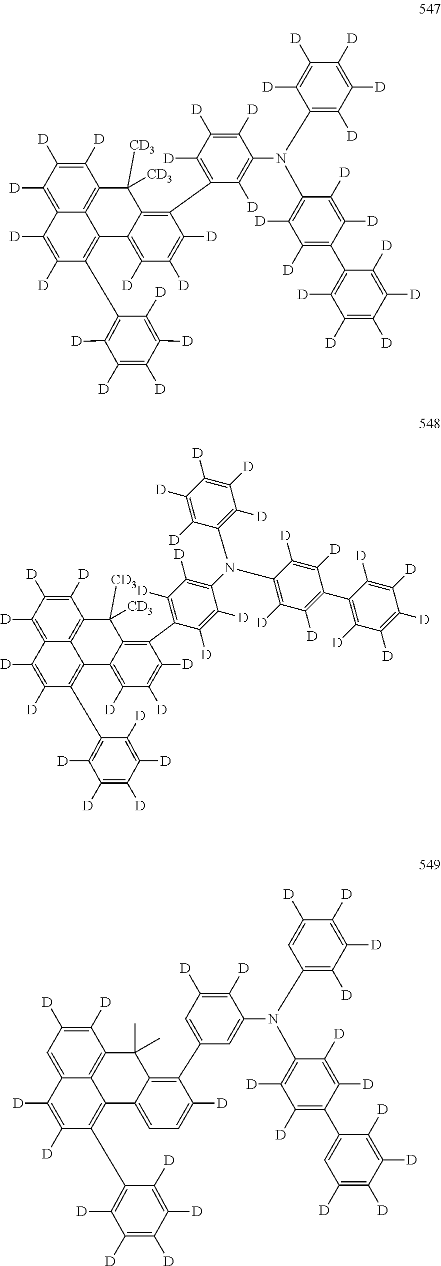

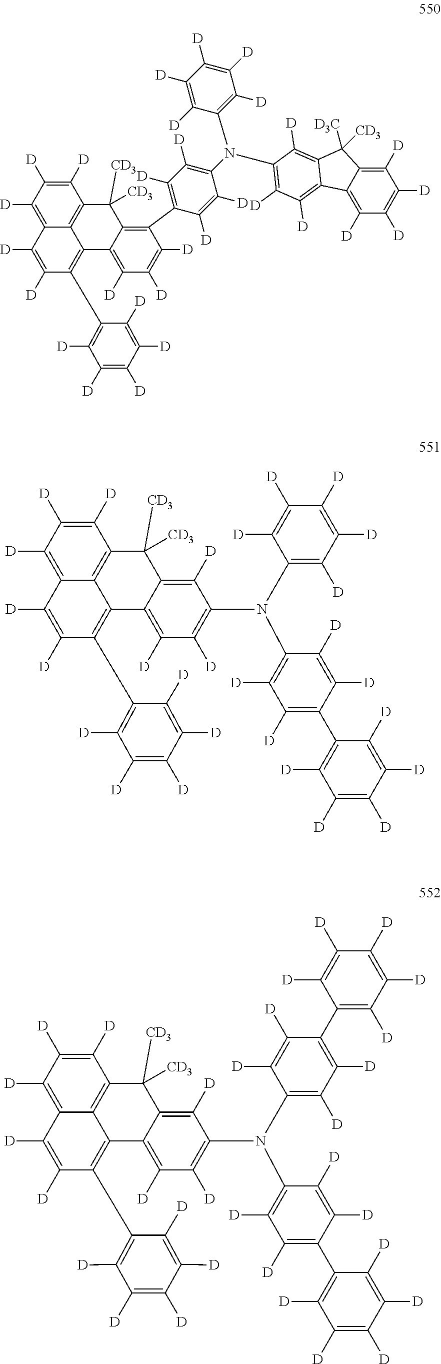

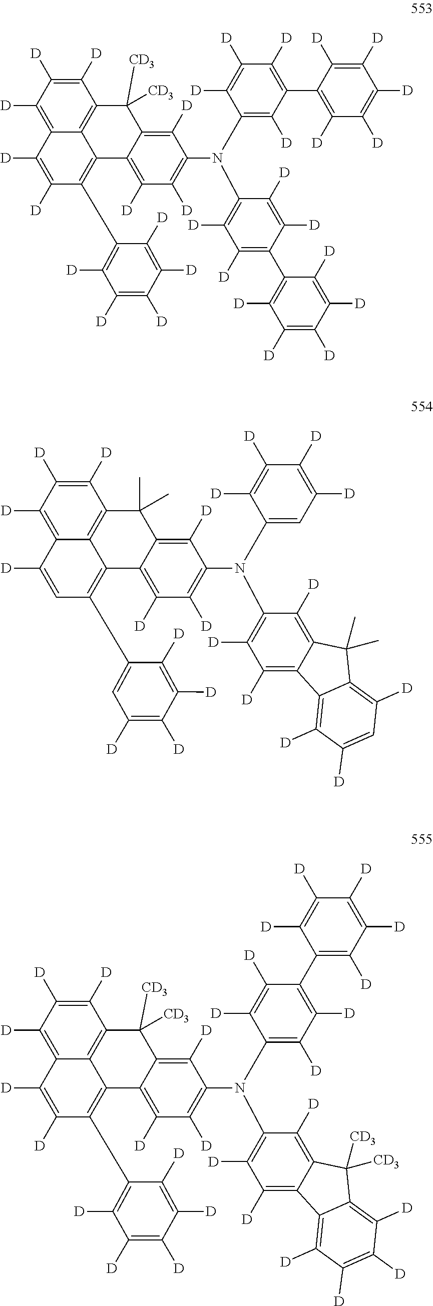

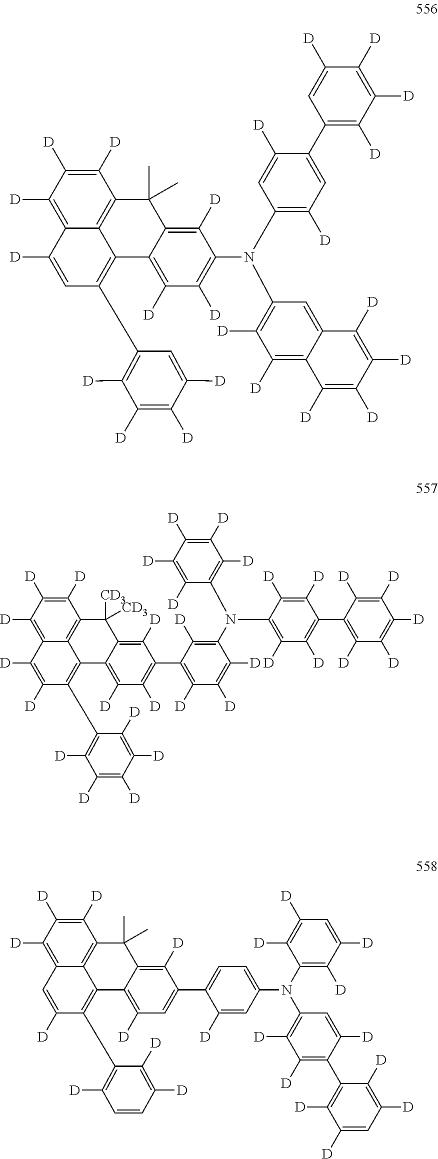

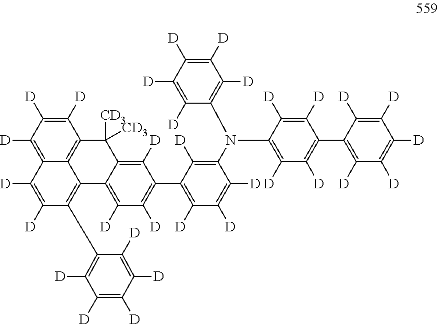 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4c([2H])c([2H])c5c(c4[2H])C(C([2H])([2H])[2H])(C([2H])([2H])[2H])c4c([2H])c([2H])c([2H])c6c([2H])c([2H])c(-c7c([2H])c([2H])c([2H])c([2H])c7[2H])c-5c46)c3[2H])c([2H])c2[2H])c([2H])c1[2H]]

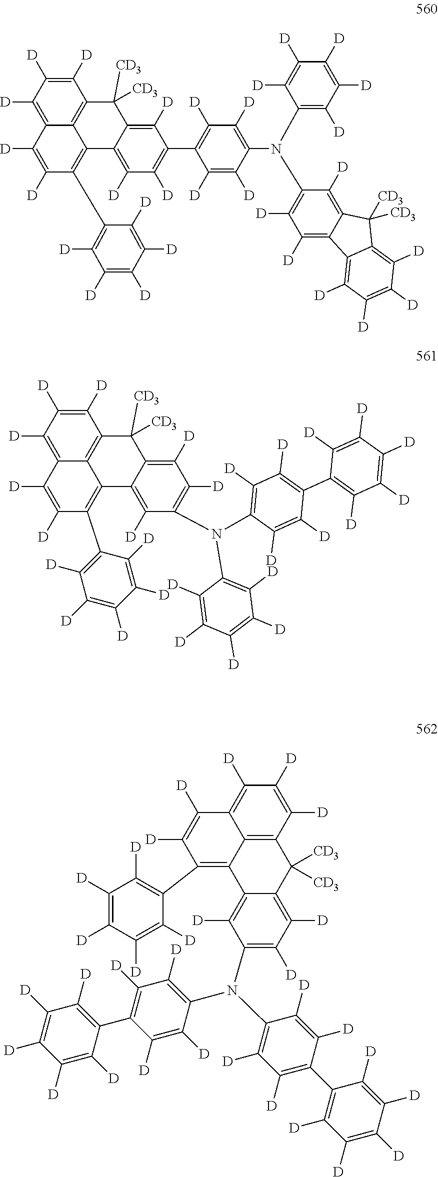

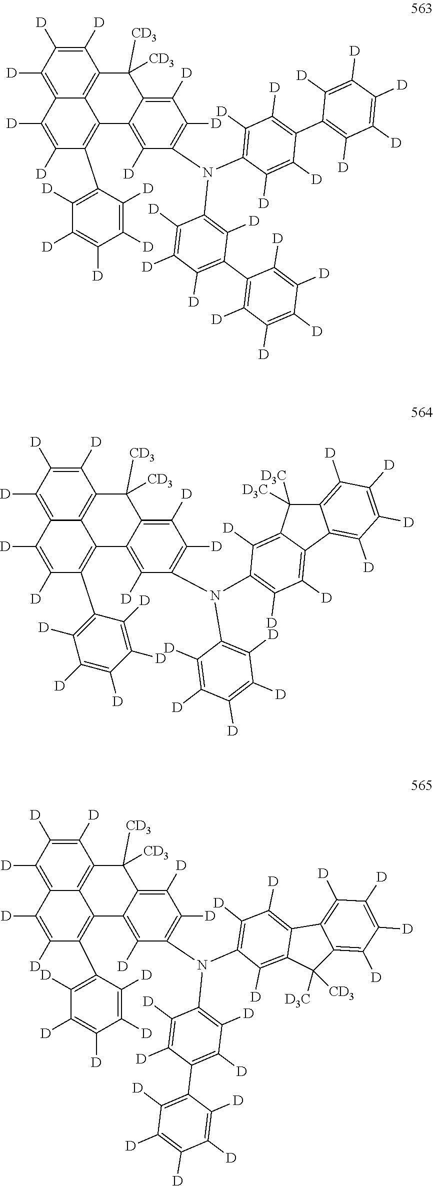

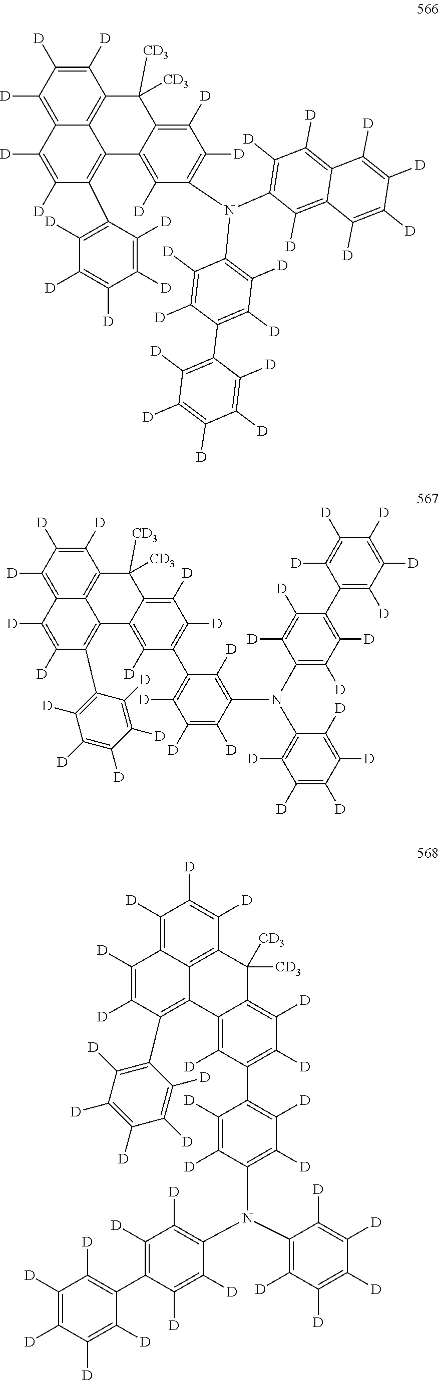

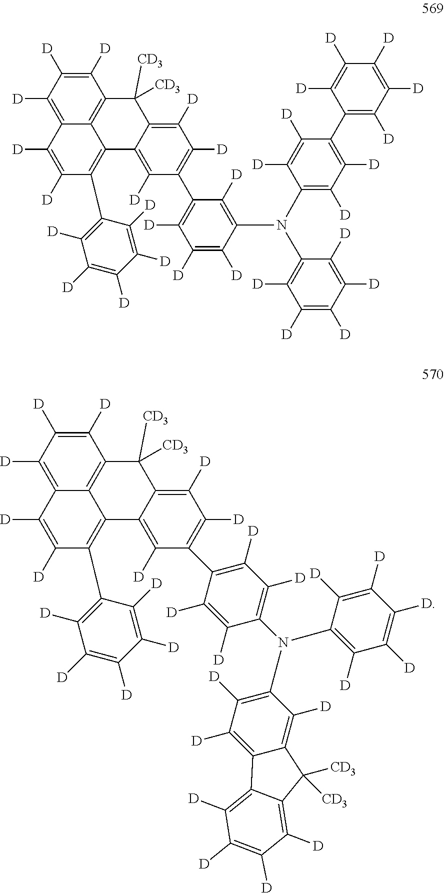

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band gap by introducing various substituents into the structure of Chemical Formula 1, and meanwhile, it is possible to improve characteristics at the interface between organic materials and diversify the use of the material.

Furthermore, in an exemplary embodiment of the present application, provided is an organic light emitting device comprising a first electrode; a second electrode provided to face the first electrode; and an organic material layer having one or more layers provided between the first electrode and the second electrode, in which one or more layers of the organic material layer comprise one or more of the heterocyclic compound according to Chemical Formula 1.

In another exemplary embodiment, provided is an organic light emitting device comprising: a first electrode; a second electrode provided to face the first electrode; and an organic material layer having one or more layers provided between the first electrode and the second electrode, in which one or more layers of the organic material layer comprise one heterocyclic compound according to Chemical Formula 1.

In still another exemplary embodiment, provided is an organic light emitting device comprising: a first electrode; a second electrode provided to face the first electrode; and an organic material layer having one or more layers provided between the first electrode and the second electrode, in which one or more layers of the organic material layer comprise two heterocyclic compounds according to Chemical Formula 1.

In the organic light emitting device, when two or more heterocyclic compounds are included, the types of heterocyclic compounds may be the same as or different from each other.

The specific content on the heterocyclic compound represented by Chemical Formula 1 is the same as that described above.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a blue organic light emitting device, and the heterocyclic compound according to Chemical Formula 1 may be used as a material for the blue organic light emitting device. For example, the heterocyclic compound according to Chemical Formula 1 may be included in a host material of a blue light emitting layer of a blue organic light emitting device.

In an exemplary embodiment of the present application, the organic light emitting device may be a green organic light emitting device, and the heterocyclic compound according to Chemical Formula 1 may be used as a material for the green organic light emitting device. For example, the heterocyclic compound according to Chemical Formula 1 may be included in a host material of a green light emitting layer of a green organic light emitting device.

In an exemplary embodiment of the present application, the organic light emitting device may be a red organic light emitting device, and the heterocyclic compound according to Chemical Formula 1 may be used as a material for the red organic light emitting device. For example, the heterocyclic compound according to Chemical Formula 1 may be included in a host material of a red light emitting layer of a red organic light emitting device.

The organic light emitting device of the present invention may be manufactured using typical manufacturing methods and materials of an organic light emitting device, except that the above-described heterocyclic compound is used to form an organic material layer having one or more layers.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic material layer of the organic light emitting device of the present invention may be composed of a single-layered structure, but may be composed of a multi-layered structure in which two or more organic material layers are stacked. For example, the organic light emitting device of the present invention may have a structure comprising a hole injection layer, a hole transport layer, a light emitting layer, an electron transport layer, an electron injection layer, and the like as organic material layers. However, the structure of the organic light emitting device is not limited thereto, and may comprise a fewer number of organic material layers.

In the organic light emitting device of the present invention, the organic material layer may comprise a light emitting layer, and the light emitting layer may comprise the heterocyclic compound.

In another organic light emitting device, the organic material layer comprises a light emitting layer, the light emitting layer comprises a host material, and the host material may comprise the heterocyclic compound.

As another example, the organic material layer comprising the heterocyclic compound comprises the heterocyclic compound represented by Chemical Formula 1 as a host, and the heterocyclic compound may be used with an iridium-based dopant.

In the organic light emitting device of the present invention, the organic material layer comprises a hole transport layer, and the hole transport layer may comprise the heterocyclic compound.

In the organic light emitting device of the present invention, the organic material layer comprises an electron injection layer or an electron transport layer, and the electron injection layer or electron transport layer may comprise the heterocyclic compound.

In another organic light emitting device, the organic material layer comprises an electron blocking layer or a hole blocking layer, and the electron blocking layer or the hole blocking layer may comprise the heterocyclic compound.

In still another organic light emitting device, the organic material layer comprises an electron blocking layer, and the electron blocking layer may comprise the heterocyclic compound.

The organic light emitting device of the present invention may further comprise one or two or more layers selected from the group consisting of a light emitting layer, a hole injection layer, a hole transport layer, an electron injection layer, an electron transport layer, an electron blocking layer, and a hole blocking layer.

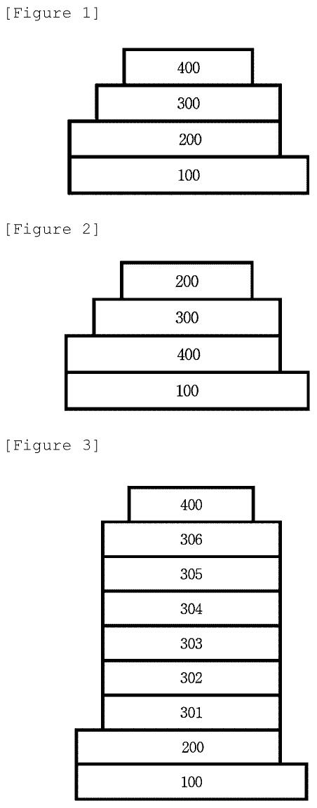

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

FIGS. 3 and 4 exemplify a case where an organic material layer is a multilayer. The organic light emitting device according to FIG. 3 comprises a hole injection layer 301, a hole transport layer 302, a light emitting layer 303, a hole blocking layer 304, an electron transport layer 305 and an electron injection layer 306, and the organic light emitting device according to FIG. 4 comprises a hole injection layer 301, a hole transport layer 302, an electron blocking layer 307, a light emitting layer 303, an electron transport layer 305 and an electron injection layer 306. However, the scope of the present application is not limited by the stacking structure as described above, and if necessary, the other layers except for the light emitting layer may be omitted, and another necessary functional layer may be further added.

An organic material layer comprising the compound of Chemical Formula 1 may additionally comprise other materials, if necessary.

Further, an exemplary embodiment of the present application provides an organic light emitting device in which an organic material layer comprising the heterocyclic compound of Chemical Formula 1 further comprises: a compound represented by the following Chemical Formula A; or a compound represented by the following Chemical Formula B.

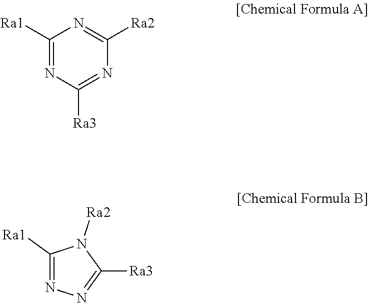

In Formulae A and B,

Ra1 to Ra3 are the same as or different from each other, and are each independently selected from the group consisting of hydrogen; deuterium; a halogen group; —CN; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; and a substituted or unsubstituted C2 to C60 heteroaryl group, or two or more adjacent groups are bonded to each other to form a substituted or unsubstituted C6 to C60 aromatic hydrocarbon ring or a substituted or unsubstituted C2 to C60 hetero ring.

In the organic light emitting device according to an exemplary embodiment of the present application, the compound represented by Chemical Formula A or Chemical Formula B may be included in the light emitting layer among the organic material layers.

In the organic light emitting device according to an exemplary embodiment of the present application, the compound represented by Chemical Formula A or Chemical Formula B may be included in the light emitting layer among the organic material layers, and specifically, may be used as a host material for the light emitting layer.

In an exemplary embodiment of the present application, the host material for the light emitting layer of the organic light emitting device may simultaneously comprise: the heterocyclic compound of Chemical Formula 1; and the compound of Chemical Formula A or the compound of Chemical Formula B.

In an exemplary embodiment of the present application, provided is a composition for an organic material layer of an organic light emitting device, comprising: the heterocyclic compound represented by Chemical Formula 1; and the compound of Chemical Formula A or the compound of Chemical Formula B.

The weight ratio of the heterocyclic compound represented by Chemical Formula 1: the compound of Chemical Formula A or the compound of Chemical Formula B in the composition may be 1:10 to 10:1, 1:8 to 8:1, 1:5 to 5:1, and 1:2 to 2:1, but is not limited thereto.

In an exemplary embodiment of the present application, provided is a method for manufacturing an organic light emitting device, the method comprising: preparing a substrate; forming a first electrode on the substrate; forming an organic material layer having one or more layers on the first electrode; and forming a second electrode on the organic material layer, in which the forming of the organic material layer comprises forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by pre-mixing the heterocyclic compound represented by Chemical Formula 1 and the compound represented by Chemical Formula A or B, and using a thermal vacuum deposition method.

The pre-mixing means that before the heterocyclic compound represented by Chemical Formula 1 and the compound represented by Chemical Formula A are deposited onto an organic material layer, the materials are first mixed and the mixture is contained in one common container and mixed. The pre-mixing means that before the heterocyclic compound represented by Chemical Formula 1 and the compound represented by Chemical Formula B are deposited onto an organic material layer, the materials are first mixed and the mixture is contained in one common container and mixed.

The pre-mixed material may be referred to as a composition for an organic material layer according to an exemplary embodiment of the present application.

In the organic light emitting device according to an exemplary embodiment of the present application, materials other than the compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material comprise: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as Zno:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material comprise: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transporting material, a pyrazoline derivative, an arylamine-based derivative, a stilbene derivative, a triphenyldiamine derivative, and the like may be used, and a low-molecular weight or polymer material may also be used.

As an electron transporting material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited or used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more materials selected from n-type host materials or p-type host materials may be used as a host material for a light emitting layer.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices comprising organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### PREPARATION EXAMPLES

### <Preparation Example 1> Preparation of Compound 002-P

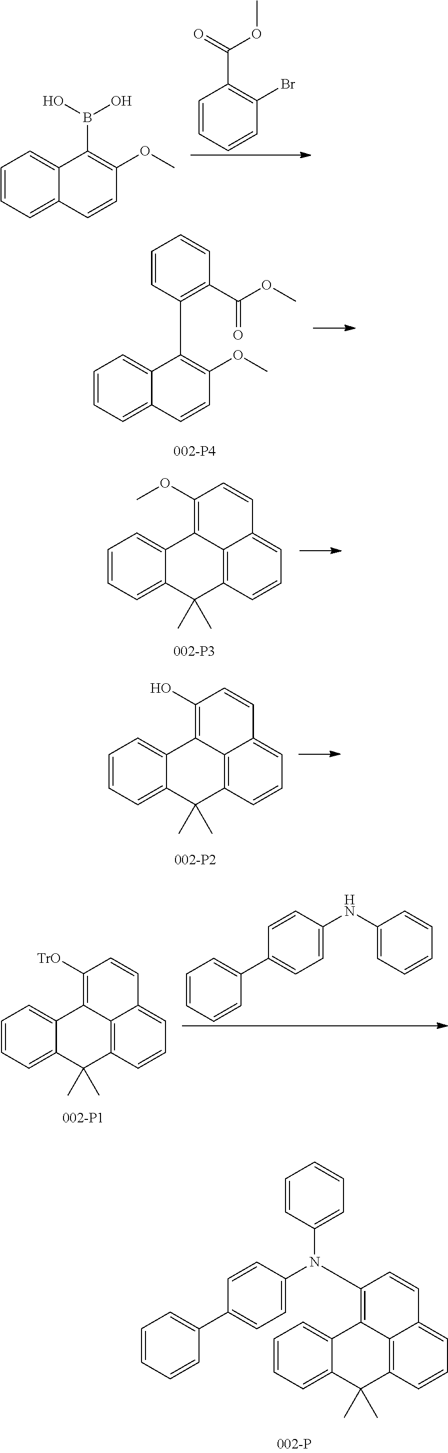

### 1) Preparation of Compound 002-P4

After Compounds (2-methoxynaphthalen-1-yl) boronic acid (100 g, 495.02 mmol) and methyl-2-bromobenzoate (106.45 g, 495.02 mmol) were dissolved in 1,000 ml of toluene, 200 ml of ethanol and 200 ml of distilled water, Pd(PPh₃)₄ (11.44 g, 9.90 mmol) and K₂CO₃ (171.04 g, 1,237.56 mmol) were added thereto and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 002-P4 (130 g, 90%).

### 2) Preparation of Compound 002-P3

After Compound 002-P4 (130 g, 444.70 mmol) was dissolved in tetrahydrofuran (1,500 ml), methylmagnesium bromide (3M solution in ether, 445 ml, 1,334 mmol) was slowly added thereto at 0° C., and then the resulting mixture was stirred at 60° C. for 6 hours. After the reaction was completed, and then terminated by adding water to the reaction solution, extraction was performed using dichloromethane and distilled water, the organic layer was dried over anhydrous MgSO₄, and then the solvent was removed by a rotary evaporator, and then, the residue was dissolved in dichloromethane, and then boron trifluoride diethyl etherate was added to the reactant, and then the resulting mixture was stirred at room temperature for 4 hours. After the reaction was completed, the resulting product was purified by column chromatography using dichloromethane and hexane as an eluting solvent, thereby obtaining Compound 002-P3 (91 g, 75%).

### 3) Preparation of Compound 002-P2

After Compound 002-P3 (91 g, 331.68 mmol) was dissolved in 1,000 ml of dichloromethane, boron tribromide (124.64 g, 497.52 mmol) was slowly added thereto at 0° C., and then the resulting mixture was stirred for 3 hours. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 002-P2 (82 g, 95%).

### 4) Preparation of Compound 002-P1

After Compound 002-P2 (82 g, 314.98 mmol) was dissolved in 1,000 ml of dichloromethane, triethylamine (38.25 g, 377.98 mmol) was added thereto, and then triflic anhydride (106.64 g, 377.98 mmol) was slowly added thereto at 0° C., and then the resulting mixture was stirred for 1 hour. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 002-P1 (110 g, 89%).

### 5) Preparation of Compound 002-P

After Compound 002-P1 (10 g, 25.48 mmol) and N-phenyl-[1,1′-biphenyl]-4-amine (6.25 g, 25.48 mmol) were dissolved in 100 ml of toluene, Pd₂(dba)₃ (0.47 g, 0.51 mmol), xantphos (0.74 g, 1.27 mmol), and t-BuONa (6.12 g, 63.71 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 002-P (9 g, 72%).

In the following Table 1, a target compound was synthesized by performing preparation in the same manner as in Preparation Example 1, except that Compound A in the following Table 1 was used instead of N-phenyl-[1,1′-biphenyl]-4-amine.

TABLE 1

| Compound | | | |
|---|---|---|---|
| No. | Compound A | Target compound | Yield |
| 003 | 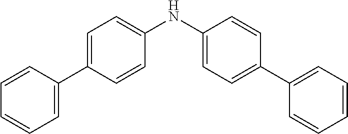 [SMILES: c1ccc(-c2ccc(Nc3ccc(-c4ccccc4)cc3)cc2)cc1] | 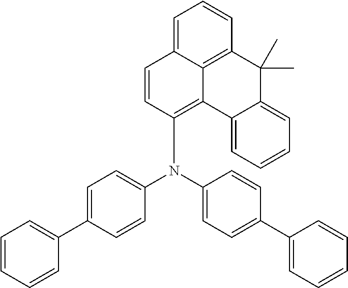 [SMILES: CC1(C)c2ccccc2-c2c(N(c3ccc(-c4ccccc4)cc3)c3ccc(-c4ccccc4)cc3)ccc3cccc1c23] | 81% |
| 007 | 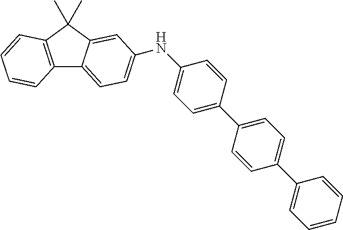 [SMILES: CC1(C)c2ccccc2-c2ccc(Nc3ccc(-c4ccc(-c5ccccc5)cc4)cc3)cc21] | 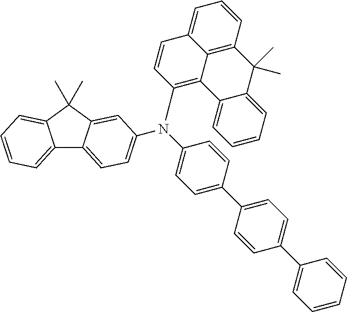 [SMILES: CC1(C)c2ccccc2-c2ccc(N(c3ccc(-c4ccc(-c5ccccc5)cc4)cc3)c3ccc4cccc5c4c3-c3ccccc3C5(C)C)cc21] | 75% |
| 008 | 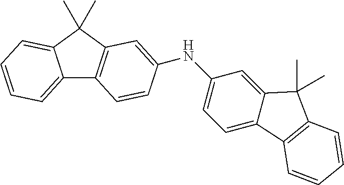 [SMILES: CC1(C)c2ccccc2-c2ccc(Nc3ccc4c(c3)C(C)(C)c3ccccc3-4)cc21] | 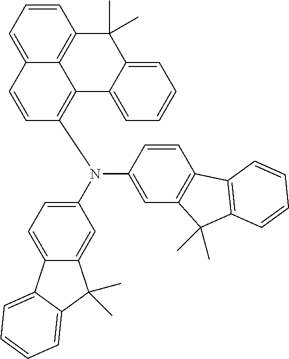 [SMILES: CC1(C)c2ccccc2-c2ccc(N(c3ccc4c(c3)C(C)(C)c3ccccc3-4)c3ccc4cccc5c4c3-c3ccccc3C5(C)C)cc21] | 69% |
| 016 | 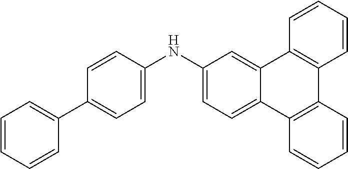 [SMILES: c1ccc(-c2ccc(Nc3ccc4c5ccccc5c5ccccc5c4c3)cc2)cc1] | 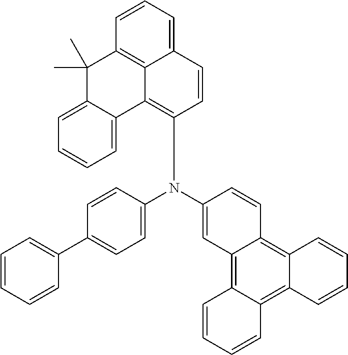 [SMILES: CC1(C)c2ccccc2-c2c(N(c3ccc(-c4ccccc4)cc3)c3ccc4c5ccccc5c5ccccc5c4c3)ccc3cccc1c23] | 77% |
| 017 | 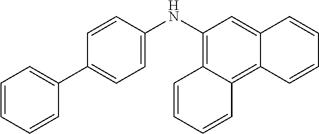 [SMILES: c1ccc(-c2ccc(Nc3cc4ccccc4c4ccccc34)cc2)cc1] | 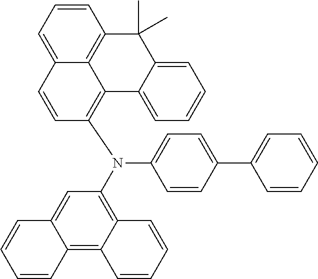 [SMILES: CC1(C)c2ccccc2-c2c(N(c3ccc(-c4ccccc4)cc3)c3cc4ccccc4c4ccccc34)ccc3cccc1c23] | 76% |
| 019 | 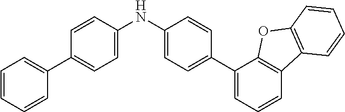 [SMILES: c1ccc(-c2ccc(Nc3ccc(-c4cccc5c4oc4ccccc45)cc3)cc2)cc1] | 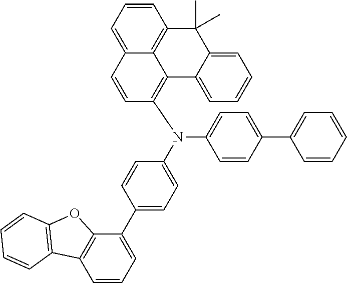 [SMILES: CC1(C)c2ccccc2-c2c(N(c3ccc(-c4ccccc4)cc3)c3ccc(-c4cccc5c4oc4ccccc45)cc3)ccc3cccc1c23] | 75% |
| 020 | 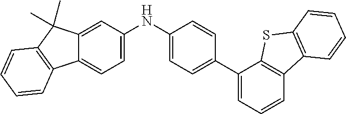 [SMILES: CC1(C)c2ccccc2-c2ccc(Nc3ccc(-c4cccc5c4sc4ccccc45)cc3)cc21] | 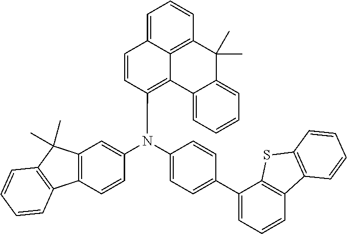 [SMILES: CC1(C)c2ccccc2-c2ccc(N(c3ccc(-c4cccc5c4sc4ccccc45)cc3)c3ccc4cccc5c4c3-c3ccccc3C5(C)C)cc21] | 80% |

### <Preparation Example 2> Preparation of Compound 026-P

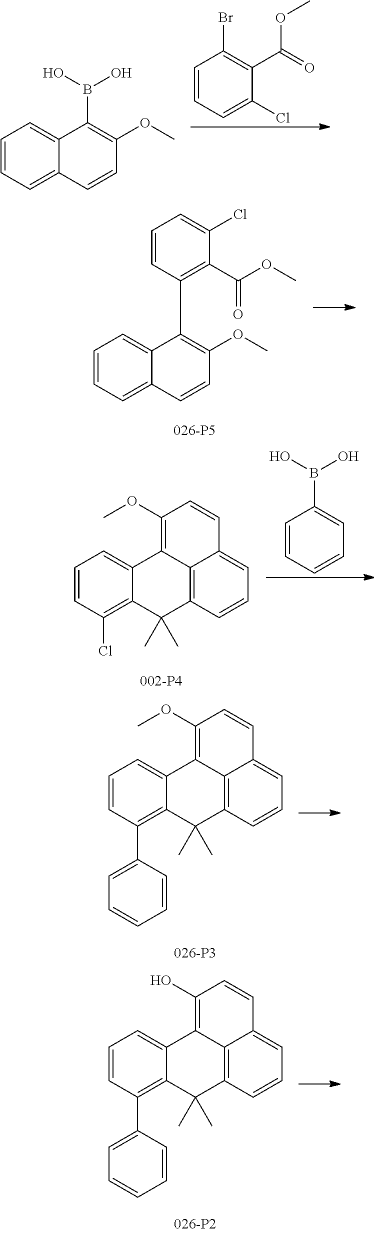

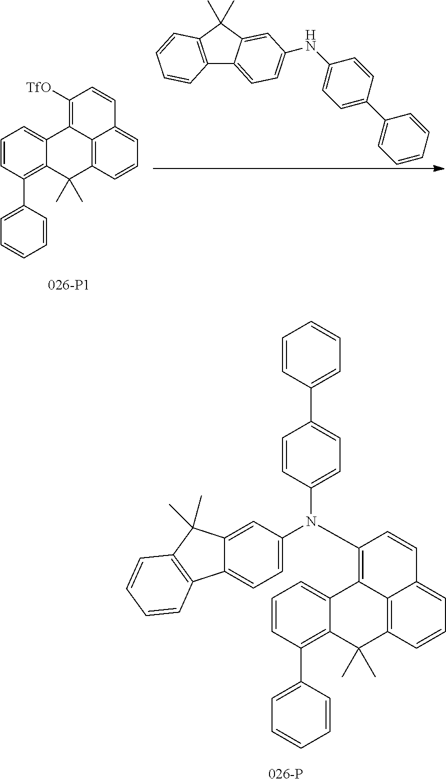 [SMILES: CC1(C)c2c(-c3ccccc3)cccc2-c2c(OS(=O)(=O)C(F)(F)F)ccc3cccc1c23 ; CC1(C)c2ccccc2-c2ccc(Nc3ccc(-c4ccccc4)cc3)cc21 ; CC1(C)c2ccccc2-c2ccc(N(c3ccc(-c4ccccc4)cc3)c3ccc4cccc5c4c3-c3cccc(-c4ccccc4)c3C5(C)C)cc21]

### 1) Preparation of Compound 026-P5

After Compounds (2-methoxynaphthalen-1-yl) boronic acid (50 g, 247.51 mmol) and methyl-2-bromo-6-chlorobenzoate (61.75 g, 247.51 mmol) were dissolved in 500 ml of toluene, 100 ml of ethanol and 100 ml of distilled water, Pd(PPh₃)₄ (5.72 g, 4.95 mmol) and K₂CO₃ (85.52 g, 618.78 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 026-P5 (69 g, 85%).

### 2) Preparation of Compound 026-P4

After Compound 026-P5 (69 g, 211.16 mmol) was dissolved in tetrahydrofuran (700 ml), methylmagnesium bromide (3M solution in ether, 211 ml, 633.47 mmol) was slowly added thereto at 0° C., and then the resulting mixture was stirred at 60° C. for 6 hours. After the reaction was completed, and then terminated by adding water to the reaction solution, extraction was performed using dichloromethane and distilled water, the organic layer was dried over anhydrous MgSO₄, and then the solvent was removed by a rotary evaporator, and then, the residue was dissolved in dichloromethane, and then boron trifluoride diethyl etherate was added to the reactant, and then the resulting mixture was stirred at room temperature for 4 hours. After the reaction was completed, the resulting product was purified by column chromatography using hexane as an eluting solvent, thereby obtaining Compound 026-P4 (50 g, 77%).

### 3) Preparation of Compound 026-P3

After Compound 026-P4 (50 g, 161.92 mmol) and phenylboronic acid (20.73 g, 170.01 mmol) were dissolved in 500 ml of 1,4-dioxane and 100 ml of distilled water, Pd(dba₂) (1.86 g, 3.24 mmol), xphos (3.86 g, 8.10 mmol) and K₂CO₃ (55.95 g, 404.79 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 026-P3 (47 g, 83%).

### 4) Preparation of Compound 026-P2

After Compound 026-P3 (47 g, 350.45 mmol) was dissolved in 500 ml of dichloromethane, boron tribromide (50.4 g, 201.17 mmol) was slowly added thereto at 0° C., and then the resulting mixture was stirred for 3 hours. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 026-P2 (43 g, 95%).

### 5) Preparation of Compound 026-P1

After Compound 026-P2 (43 g, 127.81 mmol) was dissolved in 500 ml of dichloromethane, triethylamine (15.52 g, 153.38 mmol) was added thereto, and then triflic anhydride (43.27 g, 153.38 mmol) was slowly added thereto at 0° C., and then the resulting mixture was stirred for 1 hour. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 026-P1 (52 g, 87%).

### 6) Preparation of Compound 026-P

After Compound 026-P1 (10 g, 21.35 mmol) and N-([1,1′-biphenyl]-4-yl)-9,9-dimethyl-9H-fluoren-2-amine (7.72 g, 21.35 mmol) were dissolved in 100 ml of toluene, Pd₂(dba)₃ (0.39 g, 0.43 mmol), xantphos (0.62 g, 1.07 mmol), and t-BuONa (5.13 g, 53.36 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 026-P (10 g, 69%).

In the following Table 2, a target compound was synthesized by performing preparation in the same manner as in Preparation Example 2, except that Compound B in the following Table 2 was used instead of methyl 2-bromo-6-chlorobenzoate, Compound C in the following Table 2 was used instead of phenylboronic acid, and Compound D in the following Table 2 was used instead of N-([1,1′-biphenyl]-4-yl)-9,9-dimethyl-9H-fluoren-2-amine.

TABLE 2

| Compound | Compound | Compound | | | |
|---|---|---|---|---|---|
| No. | B | C | Compound D | Target compound | Yield |
| 029 | 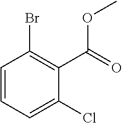 | 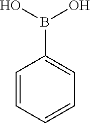 | 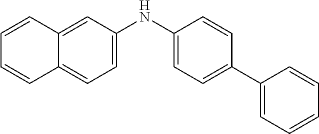 [SMILES: c1ccc(-c2ccc(Nc3ccc4ccccc4c3)cc2)cc1] | 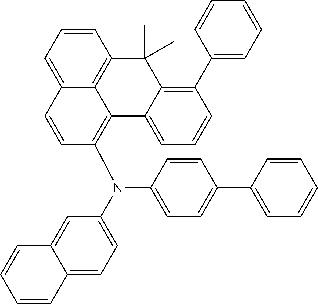 [SMILES: CC1(C)c2c(-c3ccccc3)cccc2-c2c(N(c3ccc(-c4ccccc4)cc3)c3ccc4ccccc4c3)ccc3cccc1c23] | 66% |
| 045 | 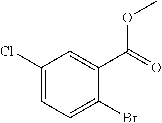 | 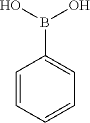 | 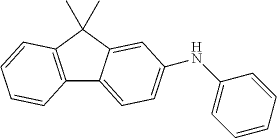 [SMILES: CC1(C)c2ccccc2-c2ccc(Nc3ccccc3)cc21] | 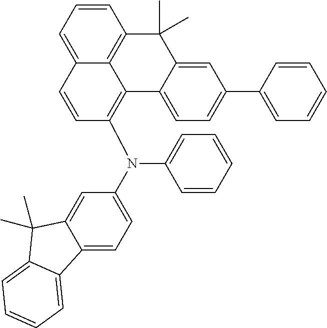 [SMILES: CC1(C)c2ccccc2-c2ccc(N(c3ccccc3)c3ccc4cccc5c4c3-c3ccc(-c4ccccc4)cc3C5(C)C)cc21] | 71% |
| 054 | 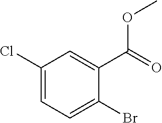 | 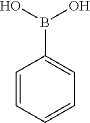 | 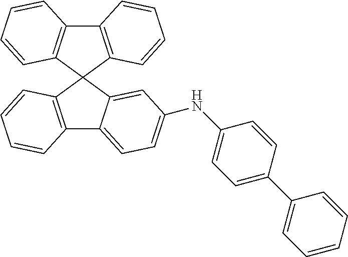 [SMILES: c1ccc(-c2ccc(Nc3ccc4c(c3)C3(c5ccccc5-c5ccccc53)c3ccccc3-4)cc2)cc1] | 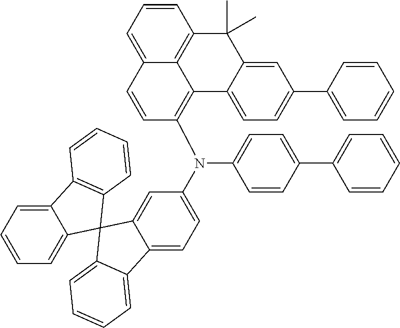 [SMILES: CC1(C)c2cc(-c3ccccc3)ccc2-c2c(N(c3ccc(-c4ccccc4)cc3)c3ccc4c(c3)C3(c5ccccc5-c5ccccc53)c3ccccc3-4)ccc3cccc1c23] | 78% |
| 059 | 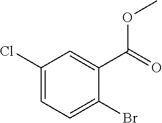 | 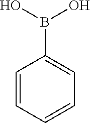 | 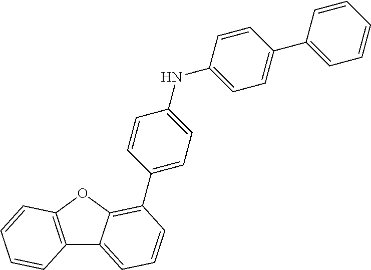 [SMILES: c1ccc(-c2ccc(Nc3ccc(-c4cccc5c4oc4ccccc45)cc3)cc2)cc1] | 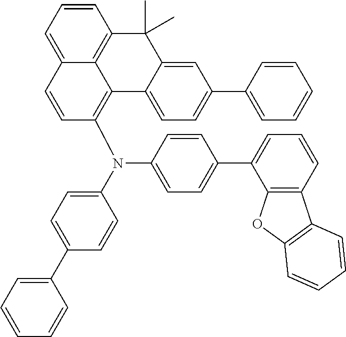 [SMILES: CC1(C)c2cc(-c3ccccc3)ccc2-c2c(N(c3ccc(-c4ccccc4)cc3)c3ccc(-c4cccc5c4oc4ccccc45)cc3)ccc3cccc1c23] | 65% |
| 077 | 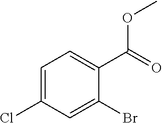 | 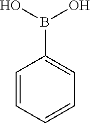 | 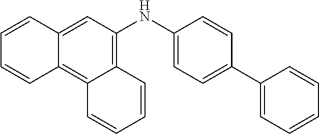 [SMILES: c1ccc(-c2ccc(Nc3cc4ccccc4c4ccccc34)cc2)cc1] | 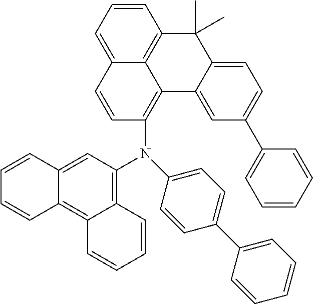 [SMILES: CC1(C)c2ccc(-c3ccccc3)cc2-c2c(N(c3ccc(-c4ccccc4)cc3)c3cc4ccccc4c4ccccc34)ccc3cccc1c23] | 75% |
| 145 | 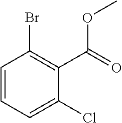 | 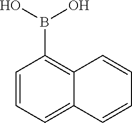 | 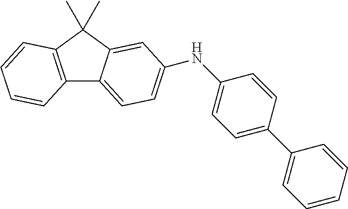 [SMILES: CC1(C)c2ccccc2-c2ccc(Nc3ccc(-c4ccccc4)cc3)cc21] | 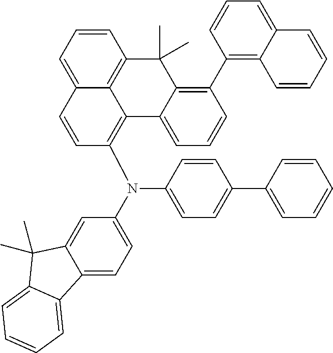 [SMILES: CC1(C)c2ccccc2-c2ccc(N(c3ccc(-c4ccccc4)cc3)c3ccc4cccc5c4c3-c3cccc(-c4cccc6ccccc46)c3C5(C)C)cc21] | 74% |
| 158 | 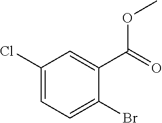 | 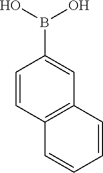 | 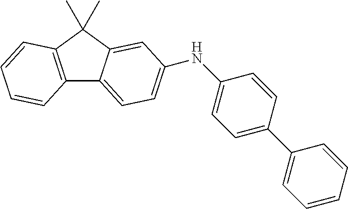 [SMILES: CC1(C)c2ccccc2-c2ccc(Nc3ccc(-c4ccccc4)cc3)cc21] | 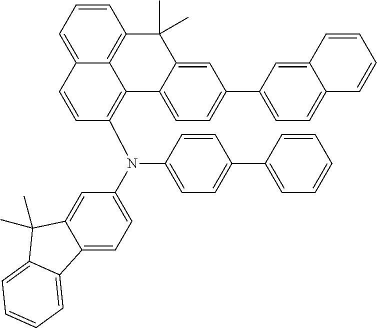 [SMILES: CC1(C)c2ccccc2-c2ccc(N(c3ccc(-c4ccccc4)cc3)c3ccc4cccc5c4c3-c3ccc(-c4ccc6ccccc6c4)cc3C5(C)C)cc21] | 79% |
| 160 | 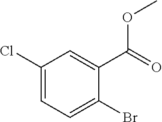 | 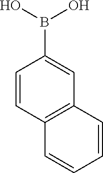 | 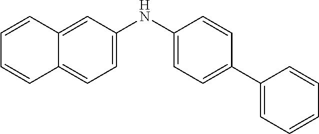 [SMILES: c1ccc(-c2ccc(Nc3ccc4ccccc4c3)cc2)cc1] | 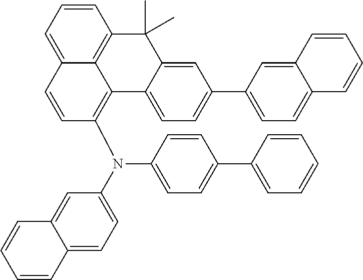 [SMILES: CC1(C)c2cc(-c3ccc4ccccc4c3)ccc2-c2c(N(c3ccc(-c4ccccc4)cc3)c3ccc4ccccc4c3)ccc3cccc1c23] | 80% |
| 170 | 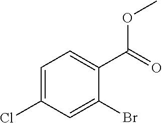 | 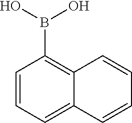 | 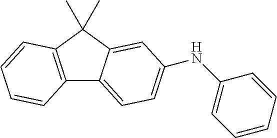 [SMILES: CC1(C)c2ccccc2-c2ccc(Nc3ccccc3)cc21] | 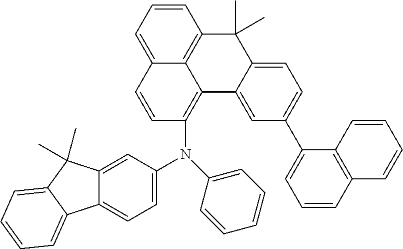 [SMILES: CC1(C)c2ccccc2-c2ccc(N(c3ccccc3)c3ccc4cccc5c4c3-c3cc(-c4cccc6ccccc46)ccc3C5(C)C)cc21] | 73% |
| 223 | 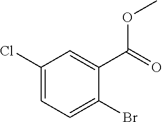 | 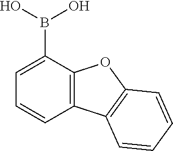 | 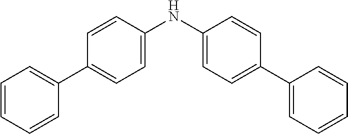 [SMILES: c1ccc(-c2ccc(Nc3ccc(-c4ccccc4)cc3)cc2)cc1] | 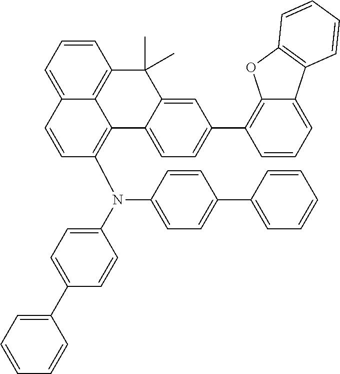 [SMILES: CC1(C)c2cc(-c3cccc4c3oc3ccccc34)ccc2-c2c(N(c3ccc(-c4ccccc4)cc3)c3ccc(-c4ccccc4)cc3)ccc3cccc1c23] | 73% |
| 479 | 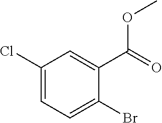 | 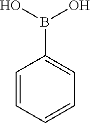 | 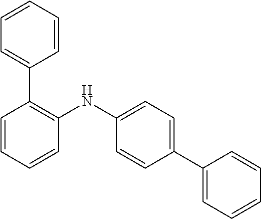 [SMILES: c1ccc(-c2ccc(Nc3ccccc3-c3ccccc3)cc2)cc1] | 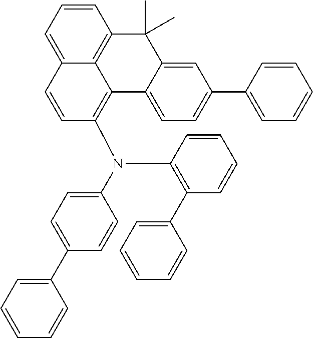 [SMILES: CC1(C)c2cc(-c3ccccc3)ccc2-c2c(N(c3ccc(-c4ccccc4)cc3)c3ccccc3-c3ccccc3)ccc3cccc1c23] | 73% |
| 480 | 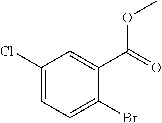 | 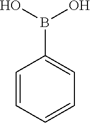 | 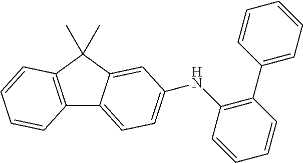 [SMILES: CC1(C)c2ccccc2-c2ccc(Nc3ccccc3-c3ccccc3)cc21] | 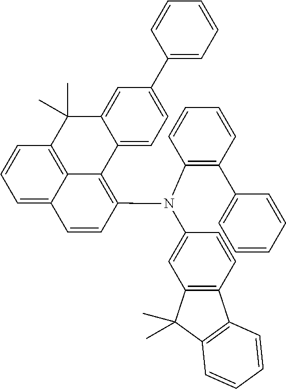 [SMILES: CC1(C)c2ccccc2-c2ccc(N(c3ccccc3-c3ccccc3)c3ccc4cccc5c4c3-c3ccc(-c4ccccc4)cc3C5(C)C)cc21] | 77% |

### <Preparation Example 3> Preparation of Compound 091-P

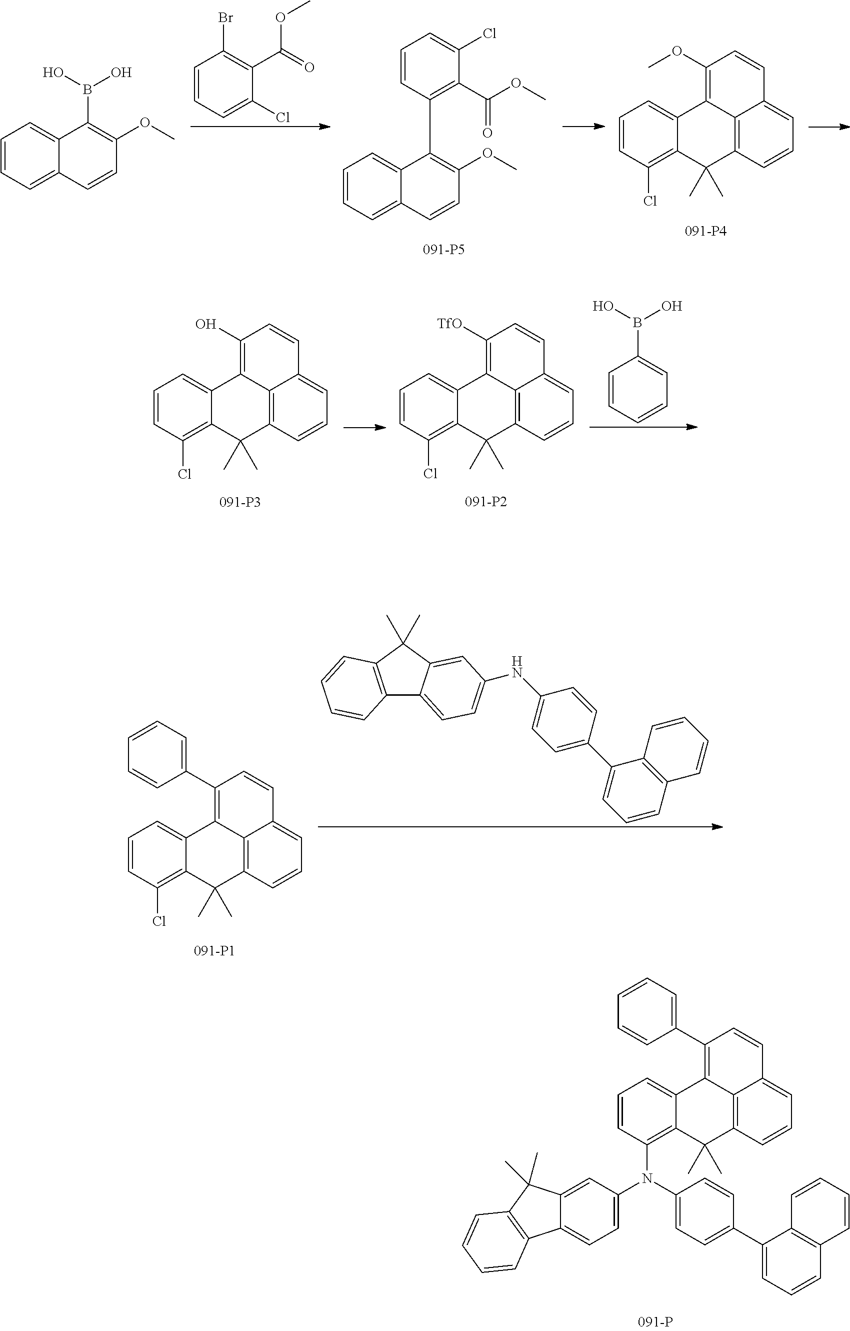

### 1) Preparation of Compound 091-P5

After Compounds (2-methoxynaphthalen-1-yl) boronic acid (50 g, 247.51 mmol) and methyl-2-bromo-6-chlorobenzoate (61.75 g, 247.51 mmol) were dissolved in 500 ml of toluene, 100 ml of ethanol and 100 ml of distilled water, Pd(PPh₃)₄ (5.72 g, 4.95 mmol) and K₂CO₃ (85.52 g, 618.78 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 091-P5 (69 g, 85%).

### 2) Preparation of Compound 091-P4

After Compound 091-P5 (69 g, 211.16 mmol) was dissolved in tetrahydrofuran (700 ml), methylmagnesium bromide (3M solution in ether, 211 ml, 633.47 mmol) was slowly added thereto at 0° C., and then the resulting mixture was stirred at 60° C. for 6 hours. After the reaction was completed, and then terminated by adding water to the reaction solution, extraction was performed using dichloromethane and distilled water, the organic layer was dried over anhydrous MgSO₄, and then the solvent was removed by a rotary evaporator, and then, the residue was dissolved in dichloromethane, and then boron trifluoride diethyl etherate was added to the reactant, and then the resulting mixture was stirred at room temperature for 4 hours. After the reaction was completed, the resulting product was purified by column chromatography using dichloromethane and hexane as an eluting solvent, thereby obtaining Compound 091-P4 (50 g, 77%).

### 3) Preparation of Compound 091-P3

After Compound 091-P4 (47 g, 350.45 mmol) was dissolved in 500 ml of dichloromethane, boron tribromide (60.85 g, 242.88 mmol) was slowly added thereto at 0° C., and then the resulting mixture was stirred for 3 hours. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 091-P3 (43 g, 90%).

### 4) Preparation of Compound 091-P2

After Compound 091-P3 (43 g, 145.88 mmol) was dissolved in 500 ml of dichloromethane, triethylamine (17.71 g, 175.05 mmol) was added thereto, and then triflic anhydride (49.39 g, 175.05 mmol) was slowly added thereto at 0° C., and then the resulting mixture was stirred for 1 hour. After the reaction was completed, and then terminated by slowly adding distilled water to the reaction solution, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 091-P2 (55 g, 88%).

### 5) Preparation of Compound 091-P1

After Compound 091-P2 (55 g, 128.85 mmol) and phenylboronic acid (16.50 g, 135.30 mmol) were dissolved in 500 ml of toluene, 100 ml of ethanol and 100 ml of distilled water, Pd(PPh₃)₄ (2.98 g, 2.58 mmol) and K₂CO₃ (44.52 g, 322.13 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 091-P1 (38 g, 83%).

### 6) Preparation of Compound 091-P

After Compound 091-P1 (10 g, 28.18 mmol) and 9,9-dimethyl-N-(4-(naphthalen-1-yl)phenyl)-9H-fluoren-2-amine (11.6 g, 28.18 mmol) were dissolved in 100 ml of toluene, Pd₂(dba)₃ (0.52 g, 0.56 mmol), xphos (0.67 g, 1.41 mmol), and t-BuONa (5.42 g, 56.36 mmol) were added thereto, and the resulting mixture was stirred under reflux for 2 hours. After the reaction was completed, dichloromethane was added to the reaction solution for dissolution, and then the resulting solution was extracted with distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 091-P (16 g, 78%).

In the following Table 3, a target compound was synthesized by performing preparation in the same manner as in Preparation Example 3, except that Compound E in the following Table 3 was used instead of methyl 2-bromo-6-chlorobenzoate, Compound F in the following Table 3 was used instead of phenylboronic acid, and Compound G in the following Table 3 was used instead of 9,9-dimethyl-N-(4-(naphthalen-1-yl)phenyl)-9H-fluoren-2-amine.

TABLE 3

| Com- | | | | | |
|---|---|---|---|---|---|
| pound | Compound | Compound | | | |
| No. | E | F | Compound G | Target compound | Yield |
| 103 | 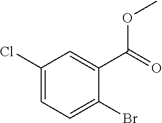 | 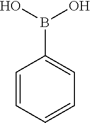 | 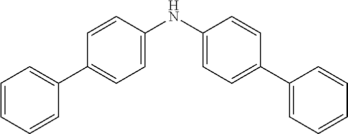 [SMILES: c1ccc(-c2ccc(Nc3ccc(-c4ccccc4)cc3)cc2)cc1] | 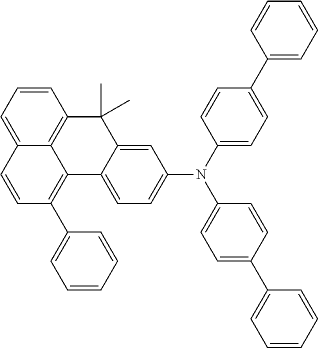 [SMILES: CC1(C)c2cc(N(c3ccc(-c4ccccc4)cc3)c3ccc(-c4ccccc4)cc3)ccc2-c2c(-c3ccccc3)ccc3cccc1c23] | 82% |
| 105 | 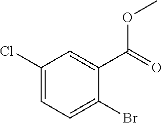 | 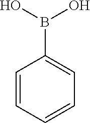 | 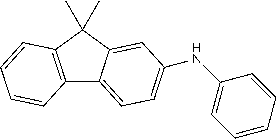 [SMILES: CC1(C)c2ccccc2-c2ccc(Nc3ccccc3)cc21] | 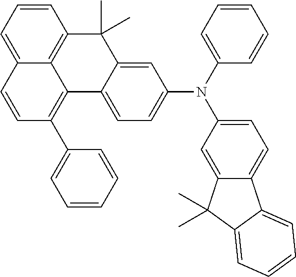 [SMILES: CC1(C)c2ccccc2-c2ccc(N(c3ccccc3)c3ccc4c(c3)C(C)(C)c3cccc5ccc(-c6ccccc6)c-4c35)cc21] | 77% |
| 106 | 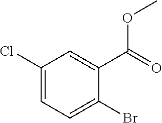 | 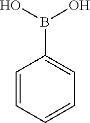 | 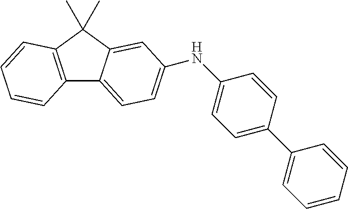 [SMILES: CC1(C)c2ccccc2-c2ccc(Nc3ccc(-c4ccccc4)cc3)cc21] | 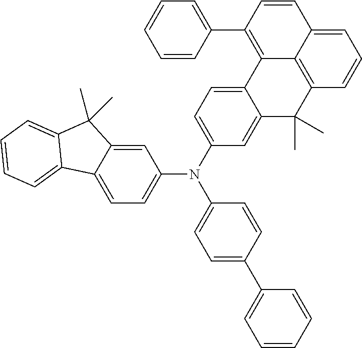 [SMILES: CC1(C)c2ccccc2-c2ccc(N(c3ccc(-c4ccccc4)cc3)c3ccc4c(c3)C(C)(C)c3cccc5ccc(-c6ccccc6)c-4c35)cc21] | 68% |
| 116 | 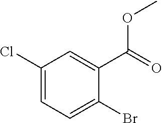 | 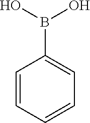 | 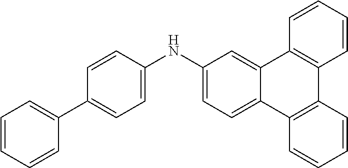 [SMILES: c1ccc(-c2ccc(Nc3ccc4c5ccccc5c5ccccc5c4c3)cc2)cc1] | 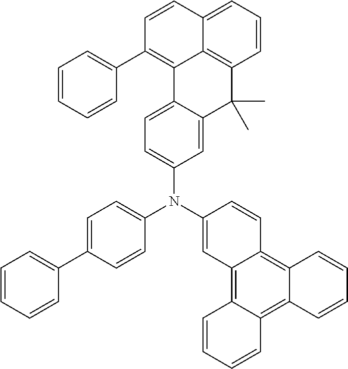 [SMILES: CC1(C)c2cc(N(c3ccc(-c4ccccc4)cc3)c3ccc4c5ccccc5c5ccccc5c4c3)ccc2-c2c(-c3ccccc3)ccc3cccc1c23] | 69% |
| 119 | 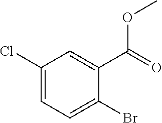 | 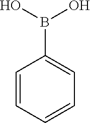 | 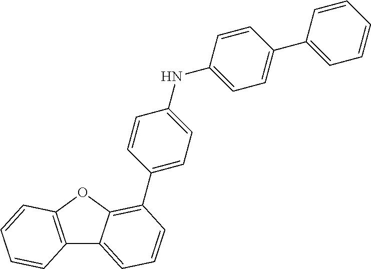 [SMILES: c1ccc(-c2ccc(Nc3ccc(-c4cccc5c4oc4ccccc45)cc3)cc2)cc1] | 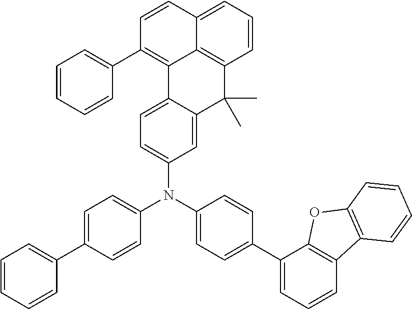 [SMILES: CC1(C)c2cc(N(c3ccc(-c4ccccc4)cc3)c3ccc(-c4cccc5c4oc4ccccc45)cc3)ccc2-c2c(-c3ccccc3)ccc3cccc1c23] | 79% |
| 122 | 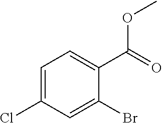 | 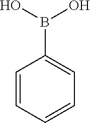 | 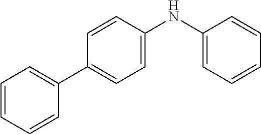 [SMILES: c1ccc(Nc2ccc(-c3ccccc3)cc2)cc1] | 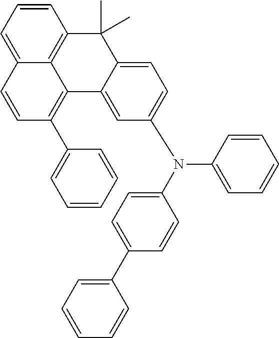 [SMILES: CC1(C)c2ccc(N(c3ccccc3)c3ccc(-c4ccccc4)cc3)cc2-c2c(-c3ccccc3)ccc3cccc1c23] | 74% |
| 131 | 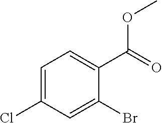 | 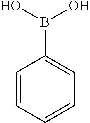 | 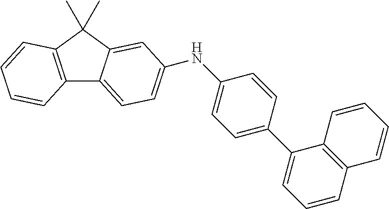 [SMILES: CC1(C)c2ccccc2-c2ccc(Nc3ccc(-c4cccc5ccccc45)cc3)cc21] | 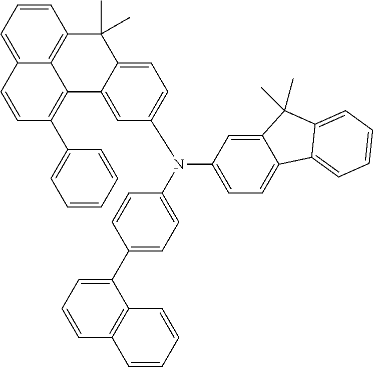 [SMILES: CC1(C)c2ccccc2-c2ccc(N(c3ccc(-c4cccc5ccccc45)cc3)c3ccc4c(c3)-c3c(-c5ccccc5)ccc5cccc(c35)C4(C)C)cc21] | 70% |
| 132 | 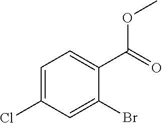 | 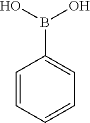 | 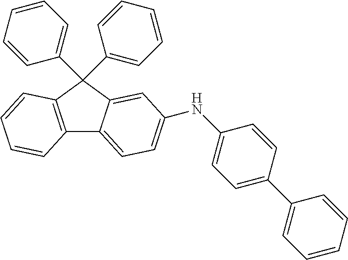 [SMILES: c1ccc(-c2ccc(Nc3ccc4c(c3)C(c3ccccc3)(c3ccccc3)c3ccccc3-4)cc2)cc1] | 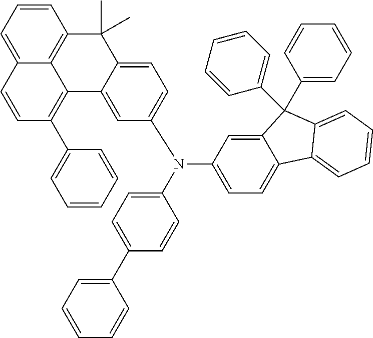 [SMILES: CC1(C)c2ccc(N(c3ccc(-c4ccccc4)cc3)c3ccc4c(c3)C(c3ccccc3)(c3ccccc3)c3ccccc3-4)cc2-c2c(-c3ccccc3)ccc3cccc1c23] | 80% |
| 133 | 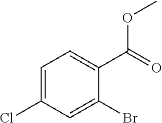 | 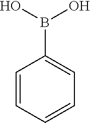 | 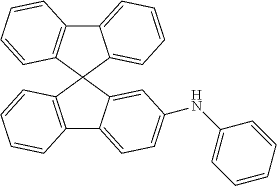 [SMILES: c1ccc(Nc2ccc3c(c2)C2(c4ccccc4-c4ccccc42)c2ccccc2-3)cc1] | 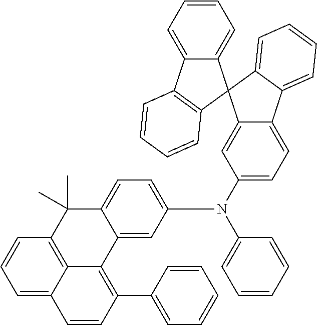 [SMILES: CC1(C)c2ccc(N(c3ccccc3)c3ccc4c(c3)C3(c5ccccc5-c5ccccc53)c3ccccc3-4)cc2-c2c(-c3ccccc3)ccc3cccc1c23] | 72% |
| 182 | 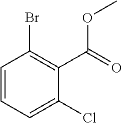 | 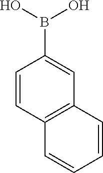 [SMILES: OB(O)c1ccc2ccccc2c1] | 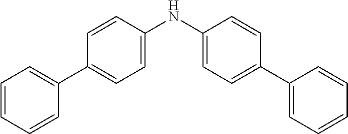 [SMILES: c1ccc(-c2ccc(Nc3ccc(-c4ccccc4)cc3)cc2)cc1] | 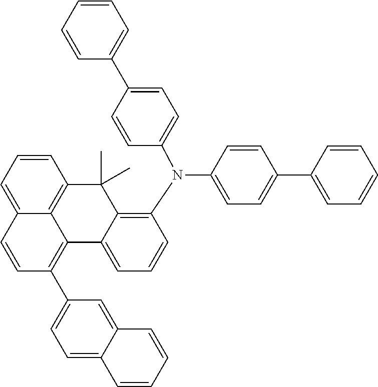 [SMILES: CC1(C)c2c(cccc2N(c2ccc(-c3ccccc3)cc2)c2ccc(-c3ccccc3)cc2)-c2c(-c3ccc4ccccc4c3)ccc3cccc1c23] | 73% |
| 183 | 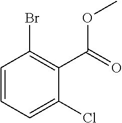 | 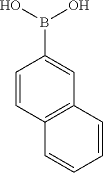 [SMILES: OB(O)c1ccc2ccccc2c1] | 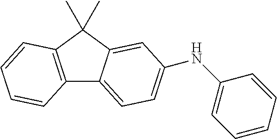 [SMILES: CC1(C)c2ccccc2-c2ccc(Nc3ccccc3)cc21] | 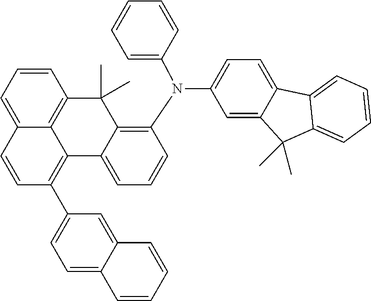 [SMILES: CC1(C)c2ccccc2-c2ccc(N(c3ccccc3)c3cccc4c3C(C)(C)c3cccc5ccc(-c6ccc7ccccc7c6)c-4c35)cc21] | 75% |
| 197 | 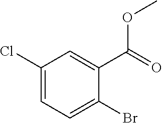 | 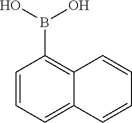 [SMILES: OB(O)c1cccc2ccccc12] | 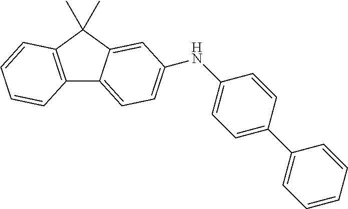 [SMILES: CC1(C)c2ccccc2-c2ccc(Nc3ccc(-c4ccccc4)cc3)cc21] | 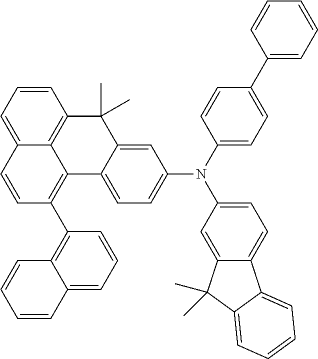 [SMILES: CC1(C)c2ccccc2-c2ccc(N(c3ccc(-c4ccccc4)cc3)c3ccc4c(c3)C(C)(C)c3cccc5ccc(-c6cccc7ccccc67)c-4c35)cc21] | 74% |
| 199 | 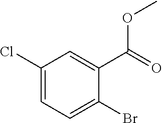 | 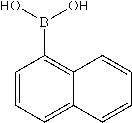 [SMILES: OB(O)c1cccc2ccccc12] | 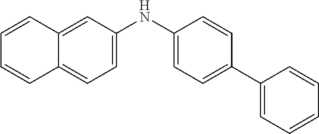 [SMILES: c1ccc(-c2ccc(Nc3ccc4ccccc4c3)cc2)cc1] | 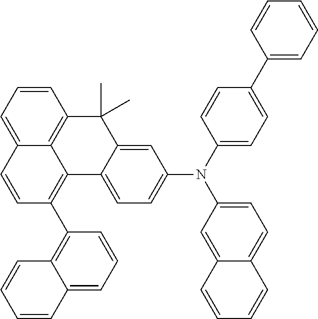 [SMILES: CC1(C)c2cc(N(c3ccc(-c4ccccc4)cc3)c3ccc4ccccc4c3)ccc2-c2c(-c3cccc4ccccc34)ccc3cccc1c23] | 66% |
| 207 | 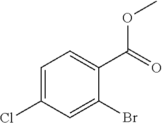 | 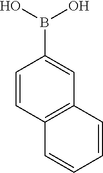 [SMILES: OB(O)c1ccc2ccccc2c1] | 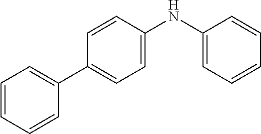 [SMILES: c1ccc(Nc2ccc(-c3ccccc3)cc2)cc1] | 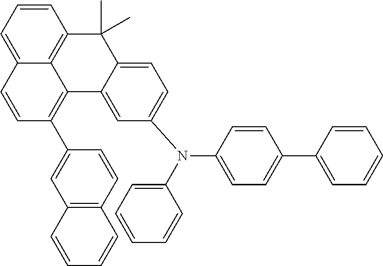 [SMILES: CC1(C)c2ccc(N(c3ccccc3)c3ccc(-c4ccccc4)cc3)cc2-c2c(-c3ccc4ccccc4c3)ccc3cccc1c23] | 75% |
| 253 | 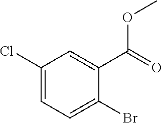 | 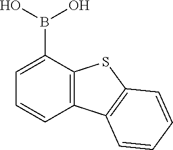 [SMILES: OB(O)c1cccc2c1sc1ccccc12] | 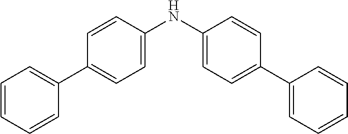 [SMILES: c1ccc(-c2ccc(Nc3ccc(-c4ccccc4)cc3)cc2)cc1] | 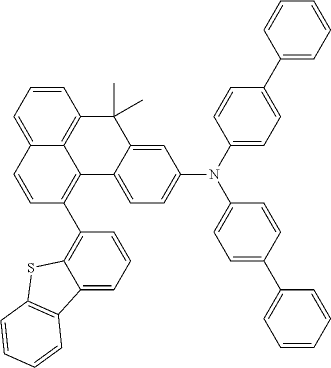 [SMILES: CC1(C)c2cc(N(c3ccc(-c4ccccc4)cc3)c3ccc(-c4ccccc4)cc3)ccc2-c2c(-c3cccc4c3sc3ccccc34)ccc3cccc1c23] | 70% |
| 486 | 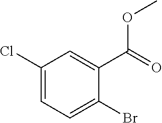 | 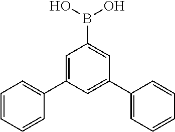 [SMILES: OB(O)c1cc(-c2ccccc2)cc(-c2ccccc2)c1] | 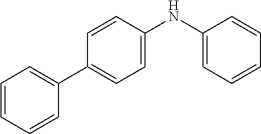 [SMILES: c1ccc(Nc2ccc(-c3ccccc3)cc2)cc1] | 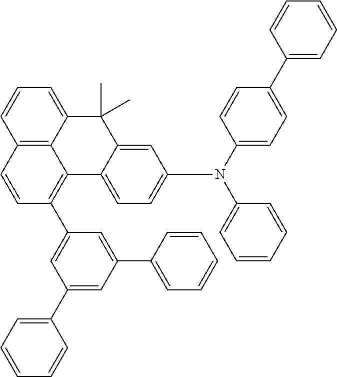 [SMILES: CC1(C)c2cc(N(c3ccccc3)c3ccc(-c4ccccc4)cc3)ccc2-c2c(-c3cc(-c4ccccc4)cc(-c4ccccc4)c3)ccc3cccc1c23] | 71% |

### Preparation Example 4> Preparation of Compound 302-P

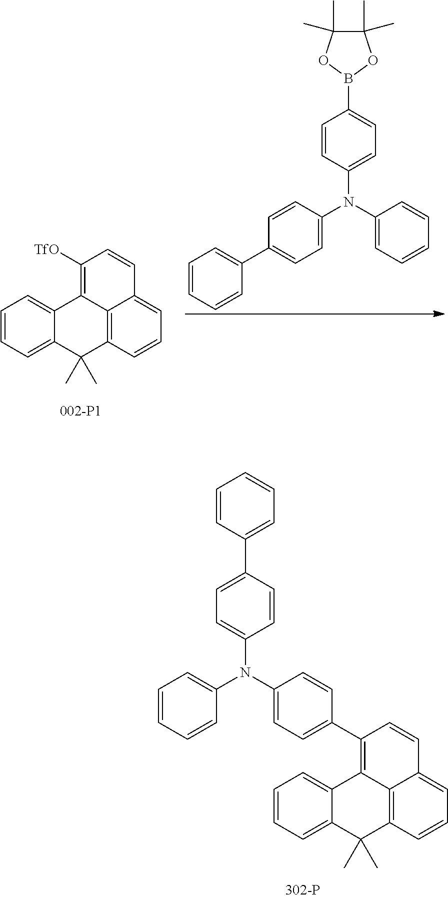

After Compound 002-P1 (10 g, 25.48 mmol) prepared in Preparation Example 1 and N-phenyl-N-(4-(4,4,5,5-tetramethyl-1,3,2-dioxaborolan-2-yl)phenyl)-[1,1′-biphenyl]-4-amine (11.97 g, 26.76 mmol) were dissolved in 100 ml of toluene, 20 ml of ethanol and 20 ml of distilled water, Pd(PPh₃)₄ (0.59 g, 0.51 mmol) and K₂CO₃ (8.81 g, 63.71 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 302-P (11 g, 77%).

In the following Table 4, a target compound was synthesized by performing preparation in the same manner as in Preparation Example 4, except that Compound H in the following Table 4 was used instead of Compound 002-P1, and Compound I in the following Table 4 was used instead of N-phenyl-N-(4-(4,4,5,5-tetramethyl-1,3,2-dioxaborolan-2-yl)phenyl)-[1,1′-biphenyl]-4-amine.

TABLE 4

| Compound | | | | |
|---|---|---|---|---|
| No. | Compound H | Compound I | Target compound | Yield |
| 319 | 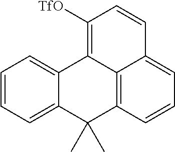 [SMILES: CC1(C)c2ccccc2-c2c(OS(=O)(=O)C(F)(F)F)ccc3cccc1c23] | 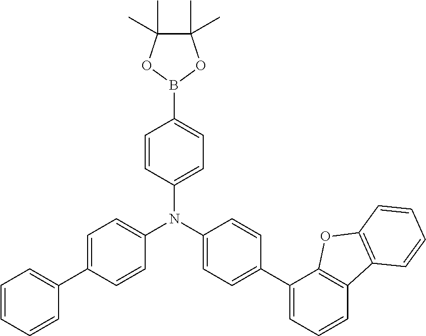 [SMILES: CC1(C)OB(c2ccc(N(c3ccc(-c4ccccc4)cc3)c3ccc(-c4cccc5c4oc4ccccc45)cc3)cc2)OC1(C)C] | 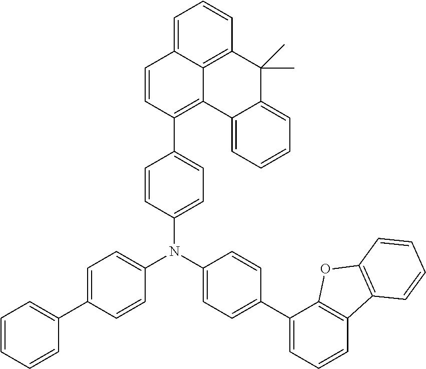 [SMILES: CC1(C)c2ccccc2-c2c(-c3ccc(N(c4ccc(-c5ccccc5)cc4)c4ccc(-c5cccc6c5oc5ccccc56)cc4)cc3)ccc3cccc1c23] | 80% |
| 332 | 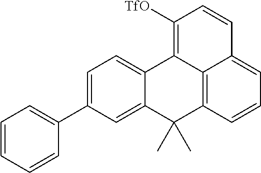 [SMILES: CC1(C)c2cc(-c3ccccc3)ccc2-c2c(OS(=O)(=O)C(F)(F)F)ccc3cccc1c23] | 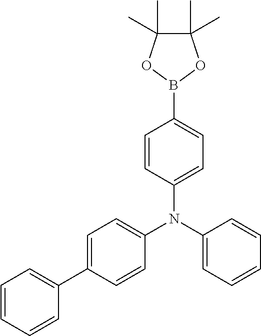 [SMILES: CC1(C)OB(c2ccc(N(c3ccccc3)c3ccc(-c4ccccc4)cc3)cc2)OC1(C)C] | 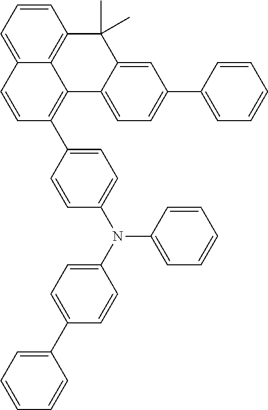 [SMILES: CC1(C)c2cc(-c3ccccc3)ccc2-c2c(-c3ccc(N(c4ccccc4)c4ccc(-c5ccccc5)cc4)cc3)ccc3cccc1c23] | 71% |
| 333 | 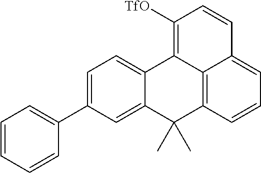 [SMILES: CC1(C)c2cc(-c3ccccc3)ccc2-c2c(OS(=O)(=O)C(F)(F)F)ccc3cccc1c23] | 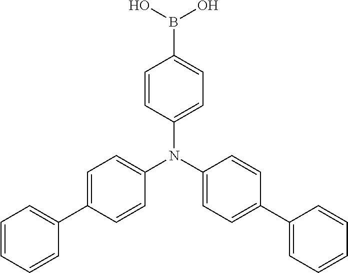 [SMILES: OB(O)c1ccc(N(c2ccc(-c3ccccc3)cc2)c2ccc(-c3ccccc3)cc2)cc1] | 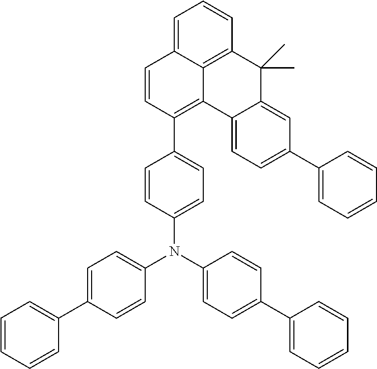 [SMILES: CC1(C)c2cc(-c3ccccc3)ccc2-c2c(-c3ccc(N(c4ccc(-c5ccccc5)cc4)c4ccc(-c5ccccc5)cc4)cc3)ccc3cccc1c23] | 65% |
| 346 | 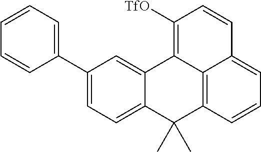 [SMILES: CC1(C)c2ccc(-c3ccccc3)cc2-c2c(OS(=O)(=O)C(F)(F)F)ccc3cccc1c23] | 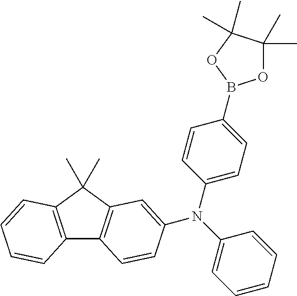 [SMILES: CC1(C)c2ccccc2-c2ccc(N(c3ccccc3)c3ccc(B4OC(C)(C)C(C)(C)O4)cc3)cc21] | 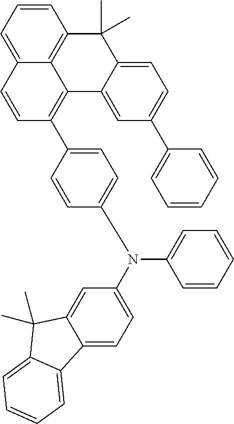 [SMILES: CC1(C)c2ccccc2-c2ccc(N(c3ccccc3)c3ccc(-c4ccc5cccc6c5c4-c4cc(-c5ccccc5)ccc4C6(C)C)cc3)cc21] | 77% |
| 381 | 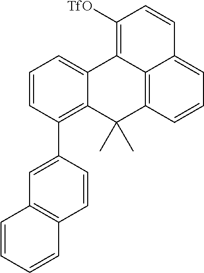 [SMILES: CC1(C)c2c(-c3ccc4ccccc4c3)cccc2-c2c(OS(=O)(=O)C(F)(F)F)ccc3cccc1c23] | 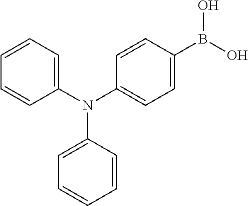 [SMILES: OB(O)c1ccc(N(c2ccccc2)c2ccccc2)cc1] | 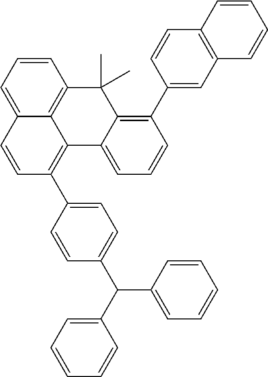 [SMILES: CC1(C)c2c(-c3ccc4ccccc4c3)cccc2-c2c(-c3ccc(C(c4ccccc4)c4ccccc4)cc3)ccc3cccc1c23] | 72% |
| 395 | 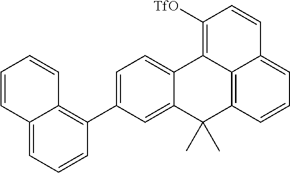 [SMILES: CC1(C)c2cc(-c3cccc4ccccc34)ccc2-c2c(OS(=O)(=O)C(F)(F)F)ccc3cccc1c23] | 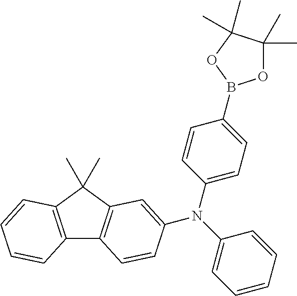 [SMILES: CC1(C)c2ccccc2-c2ccc(N(c3ccccc3)c3ccc(B4OC(C)(C)C(C)(C)O4)cc3)cc21] | 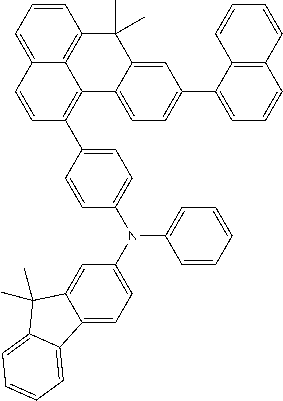 [SMILES: CC1(C)c2ccccc2-c2ccc(N(c3ccccc3)c3ccc(-c4ccc5cccc6c5c4-c4ccc(-c5cccc7ccccc57)cc4C6(C)C)cc3)cc21] | 74% |

### <Preparation Example 5> Preparation of Compound 353-P

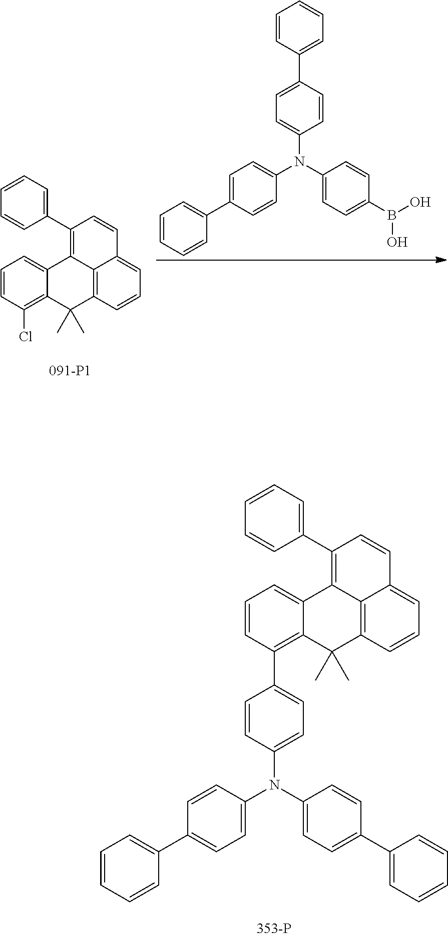

After Compound 091-P1 (10 g, 28.18 mmol) prepared in Preparation Example 3 and (4-(di([1,1′-biphenyl]-4-yl)amino)phenyl) boronic acid (13.06 g, 29.59 mmol) were dissolved in 100 ml of 1,4-dioxane and 20 ml of distilled water, Pd(dba)₂ (0.32 g, 0.56 mmol), xphos (0.67 g, 1.41 mmol) and K₂CO₃ (9.74 g, 70.45 mmol) were added thereto, and the resulting mixture was stirred under reflux for 12 hours. After the reaction was completed, the reaction solution was extracted with dichloromethane and distilled water, the organic layer was dried over anhydrous MgSO₄, and then the solvent was removed by a rotary evaporator, and then the residue was purified by column chromatography using dichloromethane and hexane as an eluting solvent, thereby obtaining Compound 353-P (17 g, 84%).

In the following Table 5, a target compound was synthesized by performing preparation in the same manner as in Preparation Example 5, except that Compound J in the following Table 5 was used instead of Compound 091-P1, and Compound K in the following Table 5 was used instead of (4-(di([1,1′-biphenyl]-4-yl)amino)phenyl) boronic acid.

TABLE 5

| Compound | | | | |
|---|---|---|---|---|
| No. | Compound J | Compound K | Target compound | Yield |
| 367 | 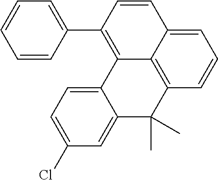 [SMILES: CC1(C)c2cc(Cl)ccc2-c2c(-c3ccccc3)ccc3cccc1c23] | 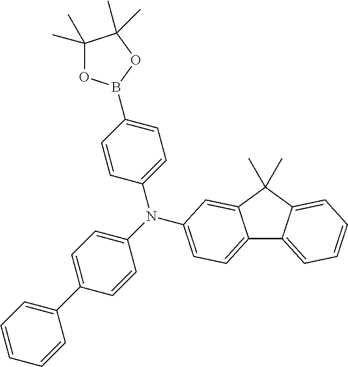 [SMILES: CC1(C)c2ccccc2-c2ccc(N(c3ccc(B4OC(C)(C)C(C)(C)O4)cc3)c3ccc(-c4ccccc4)cc3)cc21] | 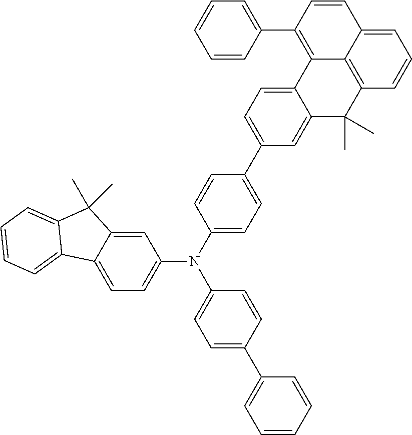 [SMILES: CC1(C)c2ccccc2-c2ccc(N(c3ccc(-c4ccccc4)cc3)c3ccc(-c4ccc5c(c4)C(C)(C)c4cccc6ccc(-c7ccccc7)c-5c46)cc3)cc21] | 70% |
| 368 | 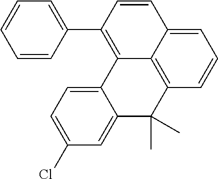 [SMILES: CC1(C)c2cc(Cl)ccc2-c2c(-c3ccccc3)ccc3cccc1c23] | 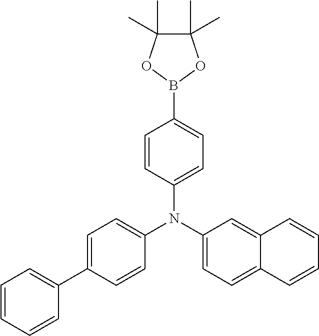 [SMILES: CC1(C)OB(c2ccc(N(c3ccc(-c4ccccc4)cc3)c3ccc4ccccc4c3)cc2)OC1(C)C] | 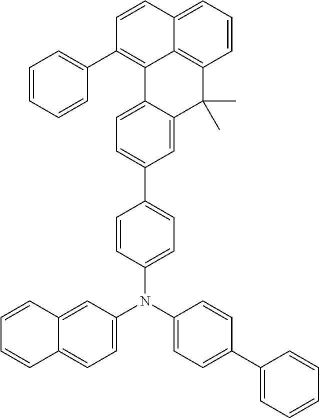 [SMILES: CC1(C)c2cc(-c3ccc(N(c4ccc(-c5ccccc5)cc4)c4ccc5ccccc5c4)cc3)ccc2-c2c(-c3ccccc3)ccc3cccc1c23] | 81% |
| 422 | 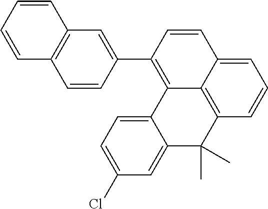 [SMILES: CC1(C)c2cc(Cl)ccc2-c2c(-c3ccc4ccccc4c3)ccc3cccc1c23] | 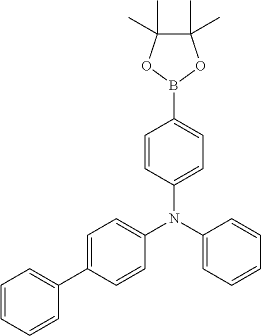 [SMILES: CC1(C)OB(c2ccc(N(c3ccccc3)c3ccc(-c4ccccc4)cc3)cc2)OC1(C)C] | 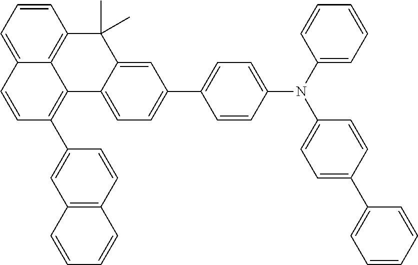 [SMILES: CC1(C)c2cc(-c3ccc(N(c4ccccc4)c4ccc(-c5ccccc5)cc4)cc3)ccc2-c2c(-c3ccc4ccccc4c3)ccc3cccc1c23] | 68% |
| 429 | 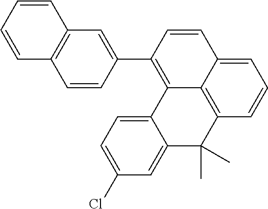 [SMILES: CC1(C)c2cc(Cl)ccc2-c2c(-c3ccc4ccccc4c3)ccc3cccc1c23] | 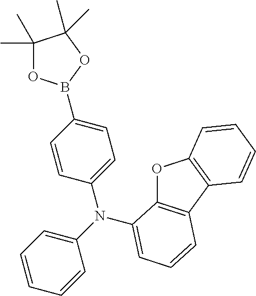 [SMILES: CC1(C)OB(c2ccc(N(c3ccccc3)c3cccc4c3oc3ccccc34)cc2)OC1(C)C] | 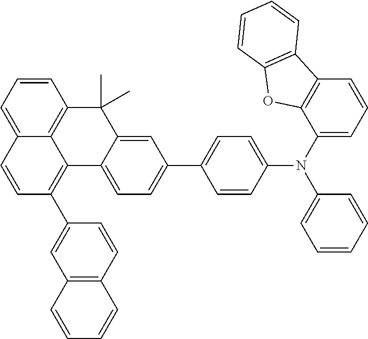 [SMILES: CC1(C)c2cc(-c3ccc(N(c4ccccc4)c4cccc5c4oc4ccccc45)cc3)ccc2-c2c(-c3ccc4ccccc4c3)ccc3cccc1c23] | 75% |
| 451 | 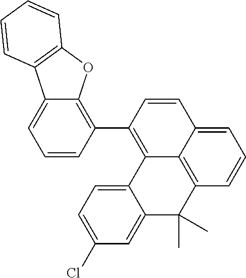 [SMILES: CC1(C)c2cc(Cl)ccc2-c2c(-c3cccc4c3oc3ccccc34)ccc3cccc1c23] | 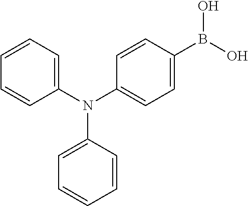 [SMILES: OB(O)c1ccc(N(c2ccccc2)c2ccccc2)cc1] | 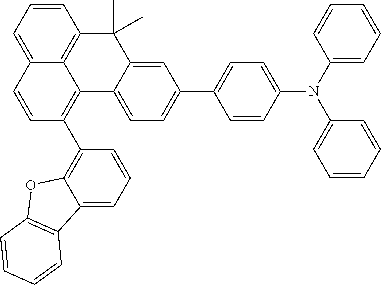 [SMILES: CC1(C)c2cc(-c3ccc(N(c4ccccc4)c4ccccc4)cc3)ccc2-c2c(-c3cccc4c3oc3ccccc34)ccc3cccc1c23] | 79% |

### <Preparation Example 6> Preparation of Compound 502-P

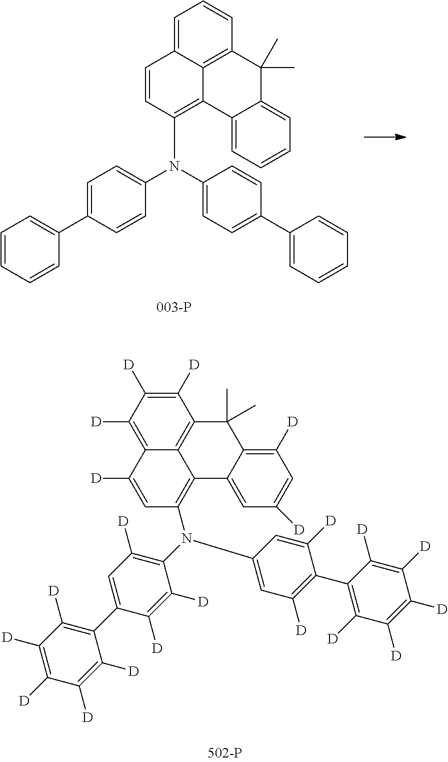 [SMILES: CC1(C)c2ccccc2-c2c(N(c3ccc(-c4ccccc4)cc3)c3ccc(-c4ccccc4)cc3)ccc3cccc1c23 ; [2H]c1cc([2H])c2c(c1)-c1c(N(c3cc([2H])c(-c4c([2H])c([2H])c([2H])c([2H])c4[2H])c([2H])c3)c3c([2H])cc(-c4c([2H])c([2H])c([2H])c([2H])c4[2H])c([2H])c3[2H])cc([2H])c3c([2H])c([2H])c([2H])c(c13)C2(C)C]

After Compound 003-P (10 g, 17.74 mmol) prepared in Preparation Example 1, trifluoromethanesulfonic acid (3.99 g, 26.61 mmol) and 100 ml De-benzene were put into a reaction flask, the resulting mixture was stirred under reflux for 5 hours. After the reaction was completed, the reaction was terminated by adding water thereto, extraction was performed using dichloromethane and distilled water, the organic layer was dried over anhydrous MgSO₄, and then the solvent was removed by a rotary evaporator, and then, the residue was purified by column chromatography using dichloromethane and hexane as an eluting solvent, thereby obtaining Compound 502-P (9 g, 87%). It was confirmed by LC/MS analysis that 21 deuteriums were substituted on average.

The compounds other than the compounds described in Preparation Examples 1 to 6 and Tables 1 to 5 were also prepared in the same manner as in the above-described Preparation Examples.

The synthetic confirmation data of the compounds prepared above are as shown in the following Tables 6 and 7.

TABLE 6

| Compound | ¹H NMR (CDCl₃, 300 MHz) |
|---|---|
| 002 | δ = 7.80 (1H, d), 7.71-7.70 (2H, m), 7.54-7.51 (6H, |
| | m), 7.41-7.33 (4H, m), 7.22-7.19 (4H, m), 6.91 (1H, |
| | d), 6.81 (1H, t), 6.69 (2H, d), 6.63 (2H, d), 1.85 |
| | (6H, s) |
| 003 | δ = 7.80 (1H, d), 7.70-7.69 (2H, m), 7.54-7.33 (17H, |
| | m), 7.22-7.19 (2H, m), 6.91 (1H, d), 6.69 (4H, d), |
| | 1.85 (6H, s) |
| 007 | δ = 7.87 (1H, d), 7.80 (1H, d), 7.70-7.69 (2H, m), |
| | 7.62-7.51 (8H, m), 7.41-7.19 (12H, m), 6.91 (1H, d), |
| | 6.75 (1H, s), 6.69 (2H, d), 6.58 (1H, d), 1.85 (6H, |
| | s), 1.72 (6H, s) |
| 008 | δ = 7.87 (2H, d), 7.80 (1H, d), 7.71-7.70 (2H, m), |
| | 7.62 (2H, d), 7.55 (1H, d), 7.38-7.19 (9H, m), 6.91 |
| | (1H, d), 6.75 (2H, d), 6.58 (2H, d), 1.85 (6H, s), |
| | 1.72 (12H, s) |
| 016 | δ = 8.93 (1H, d), 8.13-8.12 (3H, m), 7.88-7.80 (6H, |
| | m), 7.71-7.70 (2H, m), 7.54-7.33 (10H, m), 7.22-7.19 |
| | (2H, m), 7.02 (1H, d), 6.91 (1H, d), 6.69 (2H, d), |
| | 1.85 (6H, s) |
| 017 | δ = 8.93 (2H, d), 8.12 (2H, d), 7.88-7.80 (5H, m), |
| | 7.71-7.70 (2H, m), 7.54-7.33 (10H, m), 7.22-7.19 (2H, |
| | m), 6.91 (2H, d), 6.69 (2H, d), 1.85 (6H, s) |
| 019 | δ = 7.89 (1H, d), 7.85 (1H, d), 7.81-7.80 (2H, m), |
| | 7.70-7.66 (3H, m), 7.54-7.51 (8H, m), 7.41-7.32 (7H, |
| | m), 7.22-7.19 (2H, m), 6.91 (1H, d), 6.69 (4H, d), |
| | 1.85 (6H, s) |
| 020 | δ = 8.45 (1H, d), 8.41 (1H, d), 8.20 (1H, d), 7.98 |
| | (1H, d), 7.87 (1H, d), 7.80 (1H, d), 7.70-7.69 (2H, |
| | m), 7.62-7.50 (7H, m), 7.38-7.19 (7H, m), 6.91 (1H, |
| | d), 6.75 (1H, s), 6.69 (2H, d), 6.58 (1H, d), 1.85 |
| | (6H, s), 1.72 (6H, s) |
| 026 | δ = 7.87 (1H, d), 7.80 1 (1H, d), 7.70-7.62 (4H, m), |
| | 7.55-7.38 (15H, m), 7.28 (1H, t), 7.22 (1H, d), 7.19 |
| | (1H, t), 6.91 (1H, d), 6.75 (1H, s), 6.69 (2H, d), |
| | 6.58 (1H, d), 1.85 (6H, s), 1.72 (6H, s) |
| 029 | δ = 7.88-7.67 (8H, m), 7.54-7.39 (16H, m), 7.22 (1H, |
| | d), 7.19 (1H, t), 6.91 (1H, d), 6.69 (2H, d), 1.85 |
| | (6H, s) |
| 045 | δ = 7.92 (1H, s), 7.87 (1H, d), 7.80 (1H, d), 7.77 |
| | (1H, d), 7.70 (1H, d), 7.62 (1H, d), 7.55-7.51 (5H, |
| | m), 7.41-7.38 (2H, m), 7.28-7.19 (5H, m), 7.07 (1H, |
| | d), 6.91 (1H, d), 6.81 (1H, t), 6.75 (1H, s), 6.63- |
| | 6.58 (3H, m), 1.85 (6H, s), 1.72 (6H, s) |
| 054 | δ = 7.92-7.70 (7H, m), 7.55-7.16 (24H, m), 7.07 (1H, |
| | d), 6.91 (1H, d), 6.69 (2H, d), 6.55 (1H, s), 6.39 |
| | (1H, d), 1.85 (6H, s) |
| 059 | δ = 7.92-7.77 (6H, m), 7.70-7.66 (2H, m), 7.54-7.32 |
| | (17H, m), 7.22-7.19 (2H, m), 7.07 (1H, d), 6.91 (1H, |
| | d), 6.69 (4H, d), 1.85 (6H, s) |
| 077 | δ = 8.93 (2H, d), 8.12 (2H, d), 7.88-7.80 (5H, m), |
| | 7.70 (1H, d), 7.62 (1H, s), 7.54-7.41 (14H, m), 7.22- |
| | 7.19 (2H, m), 6.91 (2H, m), 6.69 (2H, d), 1.85 (6H, |
| | s) |
| 091 | δ = 8.55 (1H, d), 8.42 (1H, d), 8.08-7.87 (6H, m), |
| | 7.79 (2H, d), 7.62-7.38 (12H, m), 7.28-7.26 (2H, m), |
| | 7.07 (1H, d), 7.00 (1H, d), 6.75 (1H, s), 6.69 (2H, |
| | d), 6.58 (1H, d), 6.51 (1H, d), 1.85 (6H, s), 1.72 |
| | (6H, s) |
| 103 | δ = 8.07 (1H, d), 7.98 (1H, d), 7.92 (1H, d), 7.79 |
| | (2H, d), 7.54-7.41 (19H, m), 7.00 (1H, d), 6.69-6.68 |
| | (5H, m), 6.51 (1H, d), 1.85 (6H, s) |
| 105 | δ = 8.07 (1H, d), 7.98-7.87 (3H, m), 7.79 (2H, d), |
| | 7.62 (1H, d), 7.55-7.38 (7H, m), 7.28-7.20 (3H, m), |
| | 7.00 (1H, d), 6.81 (1H, t), 6.75 (1H, s), 6.68-6.58 |
| | (4H, m), 6.51 (1H, d), 1.85 (6H, s), 1.72 (6H, s) |
| 106 | δ = 8.07 (1H, d), 7.98-7.87 (3H, m), 7.79 (2H, d), |
| | 7.62-7.38 (15H, m), 7.28 (1H, t), 7.00 (1H, d), 6.75 |
| | (1H, s), 6.69-6.68 (3H, m), 6.58 (1H, d), 6.51 (1H, |
| | d), 1.85 (6H, s), 1.72 (6H, s) |
| 116 | δ = 8.93 (2H, d), 8.13-8.07 (4H, m), 7.98-7.79 (9H, |
| | m), 7.54-7.41 (12H, m), 7.02-7.00 (3H, m), 6.69-6.68 |
| | (3H, m), 6.51 (1H, d), 1.85 (6H, s) |
| 119 | δ = 8.07 (1H, d), 7.98-7.79 (7H, m), 7.66 (1H, d), |
| | 7.54-7.32 (17H, m), 7.00 (1H, d), 6.69-6.68 (5H, m), |
| | 6.51 (1H, d), 1.85 (6H, s) |
| 122 | δ = 8.07 (1H, d), 7.98 (1H, d), 7.92 (1H, d), 7.79 |
| | (2H, d), 7.54-7.41 (11H, m), 7.20 (2H, t), 7.11 (1H, |
| | d), 7.00 (1H, d), 6.81 (2H, m), 6.69-6.63 (4H, m), |
| | 6.51 (1H, d), 1.85 (6H, s) |
| 131 | δ = 8.55 (1H, d), 8.42 (1H, d), 8.08-7.87 (6H, m), |
| | 7.79 (2H, d), 7.62-7.38 (12H, m), 7.28 (1H, t), 7.11 |
| | (1H, d), 7.00 (1H, d), 6.81 (1H, s), 6.75 (1H, s), |
| | 6.69 (2H, d), 6.58 (1H, d), 6.51 (1H, d), 1.85 (6H, |
| | s), 1.72 (6H, s) |
| 132 | δ = 8.07 (1H, d), 7.98-7.87 (3H, m), 7.79 (2H, d), |
| | 7.62-7.26 (21H, m), 7.11 (5H, d), 7.00 (1H, d), 6.81 |
| | (1H, s), 6.75 (1H, s), 6.69 (2H, d), 6.58 (1H, d), |
| | 6.51 (1H, d), 1.85 (6H, s) |
| 133 | δ = 8.07 (1H, d), 7.98-7.87 (4H, m), 7.79-7.75 (3H, |
| | m), 7.55-7.20 (17H, m), 7.00 (1H, d), 6.81 (2H, m), |
| | 6.63 (2H, d), 6.55 (1H, s), 6.51 (1H, d), 6.39 (1H, |
| | d), 1.85 (6H, s) |
| 145 | δ = 8.55 (1H, d), 8.42 (1H, d), 8.08 (1H, d), 8.04 |
| | (1H, d), 7.87 (1H, d), 7.80 (1H, d), 7.67-7.38 (17H, |
| | m), 7.28-7.19 (3H, m), 6.91 (1H, d), 6.75 (1H, s), |
| | 6.69 (2H, d), 6.58 (1H, d), 1.85 (6H, s), 1.72 (6H, |
| | s) |
| 158 | δ = 8.00-7.70 (9H, m), 7.62-7.51 (11H, m), 7.41-7.38 |
| | (2H, m), 7.28 (3H, m), 7.07 (1H, d), 6.91 (1H, d), |
| | 6.75 (1H, s), 6.69 (2H, d), 6.58 (1H, d), 1.85 (6H, |
| | s), 1.72 (6H, s) |
| 160 | δ = 8.00-7.70 (12H, m), 7.59-7.36 (13H, m), 7.22 (1H, |
| | d), 7.19 (1H, t), 7.07 (1H, d), 6.91 (1H, d), 6.69 |
| | (2H, d), 1.85 (6H, s) |
| 170 | δ = 8.55 (1H, d), 8.42 (1H, d), 8.08 (1H, d), 8.04 |
| | (1H, d), 7.87 (1H, d), 7.80 (1H, d), 7.70 (1H, d), |
| | 7.62-7.55 (6H, m), 7.42-7.19 (8H, m), 6.91 (1H, d), |
| | 6.81-6.75 (2H, m), 6.63-6.58 (3H, m), 1.85 (6H, s), |
| | 1.72 (6H, s) |
| 182 | δ = 8.07-7.92 (6H, m), 7.73 (1H, d), 7.59-7.41 (18H, |
| | m), 7.26 (1H, t), 7.07 (1H, d), 7.00 (1H, d), 6.69 |
| | (4H, d), 6.51 (1H, d), 1.85 (6H, s) |
| 183 | δ = 8.07-7.87 (7H, m), 7.73 (1H, d), 7.62-7.55 (5H, |
| | m), 7.42 (1H, t), 7.38 (1H, t), 7.28-7.20 (4H, m), |
| | 7.07 (1H, d), 7.00 (1H, d), 6.81 (1H, t), 6.75 (1H, |
| | s), 6.63-6.58 (3H, m), 6.51 (1H, d), 1.85 (6H, s), |
| | 1.72 (6H, s) |
| 197 | δ = 8.55 (1H, d), 8.42 (1H, d), 8.07-7.87 (6H, m), |
| | 7.62-7.38 (15H, m), 7.28 (1H, t), 7.00 (1H, d), 6.75 |
| | (1H, s), 6.69 (2H, d), 6.68 (1H, s), 6.58 (1H, d), |
| | 6.51 (1H, d), 1.85 (6H, s), 1.72 (6H, s) |
| 199 | δ = 8.55 (1H, d), 8.42 (1H, d), 8.08-7.74 (9H, m), |
| | 7.54-7.36 (15H, m), 7.00 (1H, d), 6.69 (2H, d), 6.68 |
| | (1H, s), 6.51 (1H, d), 1.85 (6H, s) |
| 207 | δ = 8.07-7.92 (6H, m), 6.81 (1H, d), 7.59-7.41 (10H, |
| | m), 7.20 (2H, t), 7.11 (1H, d), 7.00 (1H, d), 6.81 |
| | (2H, m), 6.69-6.63 (4H, m), 6.51 (1H, d), 1.85 (6H, |
| | s) |
| 223 | δ = 7.92-7.77 (6H, m), 7.70 (1H, d), 7.66 (1H, d), |
| | 7.54-7.32 (17H, m), 7.22 (1H, d), 7.19 (1H, t), 7.07 |
| | (1H, d), 6.91 (1H, d), 6.69 (4H, d), 1.85 (6H, s) |
| 232 | δ = 8.07 (1H, d), 7.98-7.81 (5H, m), 7.66 (1H, d), |
| | 7.54-7.32 (12H, m), 7.20 (2H, t), 7.00 (1H, d), 6.81 |
| | (1H, t), 6.69-6.68 (3H, m), 6.63 (2H, d), 6.51 (1H, |
| | t), 1.85 (6H, s) |
| 253 | δ = 8.45 (1H, d), 8.41 (1H, d), 8.20 (1H, d), 8.07 |
| | (1H, d), 7.98 (2H, d), 7.92 (1H, d), 7.58-7.41 (19H, |
| | m), 7.00 (1H, d), 6.69-6.68 (5H, m), 6.51 (1H, t), |
| | 1.85 (6H, s) |
| 302 | δ = 8.07 (1H, d), 7.98 (1H, d), 7.92 (1H, d), 7.71 |
| | (1H, d), 7.54-7.33 (13H, m), 7.20 (2H, t), 7.00 (1H, |
| | d), 6.81 (1H, t), 6.69 (4H, d), 6.63 (2H, d), 1.85 |
| | (6H, S) |
| 319 | δ = 8.07 (1H, d), 7.98 (1H, d), 7.92 (1H, d), 7.89- |
| | 7.81 (3H, m), 7.71 (1H, d), 7.66 (1H, d), 7.54-7.32 |
| | (18H, m), 7.00 (1H, d), 6.69 (6H, d), 1.85 (6H, s) |
| 332 | δ = 8.07 (1H, d), 7.98-7.92 (3H, m), 7.77 (1H, d), |
| | 7.54-7.41 (15H, m), 7.20 (2H, t), 7.07 (1H, d), 7.00 |
| | (1H, d), 6.81 (1H, t), 6.69 (4H, d), 6.63 (2H, d), |
| | 1.85 (6H, s) |
| 333 | δ = 8.07 (1H, d), 7.98-7.92 (3H, m), 7.71 (1H, d), |
| | 7.54-7.41 (22H, m), 7.07 (1H, d), 7.00 (1H, d), 6.69 |
| | (6H, d), 1.85 (6H, s) |
| 346 | δ = 8.07 (1H, d), 7.98-7.87 (3H, m), 7.62-7.28 (17H, |
| | m), 7.00 (1H, d), 6.81-6.58 (7H, m), 1.85 (6H, s), |
| | 1.72 (6H, s) |
| 353 | δ = 8.07 (1H, d), 7.98 (1H, d), 7.92 (1H, d), 7.79 |
| | (2H, d), 7.67 (2H, d), 7.54-7.39 (21H, m), 7.00 (1H, |
| | d), 6.69 (6H, d), 1.85 (6H, s) |
| 367 | δ = 8.07 (1H, d), 7.98-7.87 (4H, m), 7.79 (2H, d), |
| | 7.62-7.38 (16H, m), 7.28 (1H, t), 7.07 (1H, d), 7.00 |
| | (1H, d), 6.75 (1H, s), 6.69 (4H, d), 6.58 (1H, d), |
| | 1.85 (6H, s), 1.72 (6H, s) |
| 368 | δ = 8.07 (1H, d), 7.98-7.74 (10H, m), 7.54-7.36 (16H, |
| | m), 7.07 (1H, d), 7.00 (1H, d), 6.69 (4H, d), 1.85 |
| | (6H, s) |
| 381 | δ = 8.07-7.92 (6H, m), 7.73-7.54 (8H, m), 7.42-7.39 |
| | (2H, m), 7.20 (4H, t), 7.00 (1H, d), 6.81 (2H, t), |
| | 6.69 (2H, d), 6.63 (4H, d), 1.85 (6H, s) |
| 395 | δ = 8.55 (1H, d), 8.42 (1H, d), 8.08-7.87 (7H, m), |
| | 7.77 (1H, d), 7.62-7.54 (7H, m), 7.42 (1H, t), 7.38 |
| | (1H, t), 7.28-7.20 (3H, m), 7.07-7.00 (2H, m), 6.81- |
| | 6.58 (7H, m), 1.85 (6H, s), 1.72 (6H, s) |
| 422 | δ = 8.07-7.92 (7H, m), 7.77-7.73 (2H, m), 7.59-7.41 |
| | (13H, m), 7.20 (2H, t), 7.07 (1H, d), 7.00 (1H, d), |
| | 6.81 (1H, t), 6.69 (4H, d), 6.63 (2H, d), 1.85 (6H, |
| | s) |
| 429 | δ = 8.07-7.89 (8H, m), 7.77-7.54 (8H, m), 7.42-7.20 |
| | (6H, m), 7.07-7.00 (3H, m), 6.81 (1H, t), 6.69 (2H, |
| | d), 6.63 (2H, d), 6.39 (1H, d), 1.85 (6H, s) |
| 451 | δ = 8.07 (1H, d), 7.98-7.77 (7H, m), 7.66 (1H, d), |
| | 7.54 (2H, d), 7.42-7.32 (4H, m), 7.20 (4H, t), 7.07 |
| | (1H, d), 7.00 (1H, d), 6.81 (2H, t), 6.69 (2H, d), |
| | 6.63 (4H, d), 1.85 (6H, s) |
| 479 | δ = 7.92 (1H, s), 7.80-7.77 (2H, m), 7.70 (1H, d), |
| | 7.54-7.41 (16H, m), 7.19-7.07 (6H, m), 6.91 (1H, d), |
| | 6.87 (1H, t), 6.69 (3H, d), 1.85 (6H, s) |
| 480 | δ = 7.92-7.77 (4H, m), 7.70 (1H, d), 7.62 (1H, d), |
| | 7.55-7.38 (11H, m), 7.28-7.07 (7H, m), 6.91 (1H, d), |
| | 6.87 (1H, t), 6.75 (1H, s), 6.69 (1H, d), 6.58 (1H, |
| | d), 1.85 (6H, s), 1.72 (6H, s) |
| 486 | δ = 8.07 (1H, d), 7.98 (1H, d), 7.92 (1H, d), 7.66 |
| | (3H, s), 7.54-7.41 (19H, m), 7.20 (2H, t), 7.00 (1H, |
| | d), 6.81 (1H, t), 6.69-6.63 (5H, m), 6.51 (1H, d), |
| | 1.85 (6H, s) |

TABLE 7

| Com- | | Com- | |
|---|---|---|---|
| pound | FD-MS | pound | FD-MS |
| 002 | m/z = 487.63 | 003 | m/z = 563.73 |
| | (C₃₇H₂₉N = 487.23) | | (C₄₃H₃₃N = 563.26) |
| 007 | m/z = 679.89 | 008 | m/z = 643.86 |
| | (C₅₂H₄₁N = 689.32) | | (C₄₉H₄₁N = 643.32) |
| 016 | m/z = 637.81 | 017 | m/z = 587.75 |
| | (C₄₉H₃₅N = 637.28) | | (C₄₅H₃₃N = 587.26) |
| 019 | m/z = 653.81 | 020 | m/z = 709.94 |
| | (C₄₉H₃₅NO = 635.27) | | (C₅₂H₃₉NS = 709.28) |
| 026 | m/z = 679.89 | 029 | m/z = 613.79 |
| | (C₅₂H₄₁N = 689.32) | | (C₄₇H₃₅N = 613.28) |
| 045 | m/z = 603.79 | 054 | m/z = 802.01 |
| | (C₄₆H₃₇N = 603.29) | | (C₆₂H₄₃N = 801.34) |
| 059 | m/z = 729.90 | 077 | m/z = 663.85 |
| | (C₅₅H₃₉NO = 729.30) | | (C₅₁H₃₇N = 663.29) |
| 091 | m/z = 729.95 | 103 | m/z = 639.82 |
| | (C₅₆H₄₃N = 729.34) | | (C₄₉H₃₇N = 639.29) |
| 105 | m/z = 603.79 | 106 | m/z = 679.89 |
| | (C₄₆H₃₇N = 603.29) | | (C₅₂H₄₁N = 689.32) |
| 116 | m/z = 713.90 | 119 | m/z = 729.90 |
| | (C₅₅H₃₉N = 713.31) | | (C₅₅H₃₉NO = 729.30) |
| 122 | m/z = 563.73 | 131 | m/z = 729.95 |
| | (C₄₃H₃₃N = 563.26) | | (C₅₆H₄₃N = 729.34) |
| 132 | m/z = 804.03 | 133 | m/z = 725.92 |
| | (C₆₂H₄₅N = 803.36) | | (C₅₆H₃₉N = 725.31) |
| 145 | m/z = 729.95 | 158 | m/z = 729.95 |
| | (C₅₆H₄₃N = 729.34) | | (C₅₆H₄₃N = 729.34) |
| 160 | m/z = 663.85 | 170 | m/z = 653.85 |
| | (C₅₁H₃₇N = 663.29) | | (C₅₀H₃₉N = 653.31) |
| 182 | m/z = 689.88 | 183 | m/z = 653.85 |
| | (C₅₃H₃₉N = 689.31) | | (C₅₀H₃₉N = 653.31) |
| 197 | m/z = 729.95 | 199 | m/z = 663.85 |
| | (C₅₆H₄₃N = 729.34) | | (C₅₁H₃₇N = 663.29) |
| 207 | m/z = 613.79 | 223 | m/z = 729.90 |
| | (C₄₇H₃₅N = 613.28) | | (C₅₅H₃₉NO = 729.30) |
| 232 | m/z = 653.81 | 253 | m/z = 745.97 |
| | (C₄₉H₃₅NO = 635.27) | | (C₅₅H₃₉NS = 745.28) |
| 302 | m/z = 563.73 | 319 | m/z = 729.90 |
| | (C₄₃H₃₃N = 563.26) | | (C₅₅H₃₉NO = 729.30) |
| 332 | m/z = 639.82 | 333 | m/z = 715.92 |
| | (C₄₉H₃₇N = 639.29) | | (C₅₅H₄₁N = 715.32) |
| 346 | m/z = 679.89 | 353 | m/z = 715.92 |
| | (C₅₂H₄₁N = 689.32) | | (C₅₅H₄₁N = 715.32) |
| 367 | m/z = 755.98 | 368 | m/z = 689.88 |
| | (C₅₈H₄₅N = 755.36) | | (C₅₃H₃₉N = 689.31) |
| 381 | m/z = 613.79 | 395 | m/z = 729.95 |
| | (C₄₇H₃₅N = 613.28) | | (C₅₆H₄₃N = 729.34) |
| 422 | m/z = 689.88 | 429 | m/z = 703.87 |
| | (C₅₃H₃₉N = 689.31) | | (C₅₃H₃₇NO = 703.29) |
| 451 | m/z = 653.81 | 479 | m/z = 639.82 |
| | (C₄₉H₃₅NO = 635.27) | | (C₄₉H₃₇N = 639.29) |
| 480 | m/z = 679.89 | 486 | m/z = 715.92 |
| | (C₅₂H₄₁N = 689.32) | | (C₅₅H₄₁N = 715.32) |

### EXPERIMENTAL EXAMPLES

### Experimental Example 1

### (1) Manufacture of Organic Light Emitting Device

### Comparative Example 1

A glass substrate, in which ITO was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

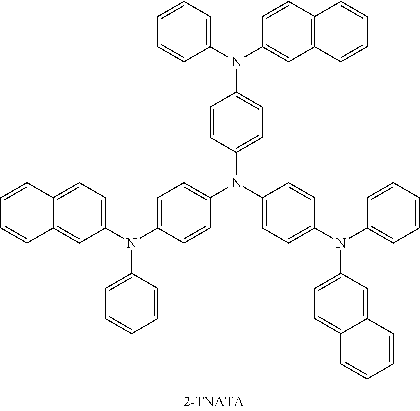 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10-6 torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N, N′-bis (α-naphthyl)-N, N′-diphenyl-4,4′-diamine (NPB) in another cell in the vacuum deposition apparatus and applying current to the cell to evaporate NPB.

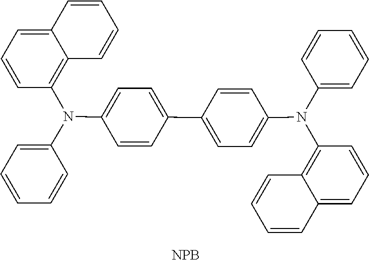 [SMILES: c1ccc(N(c2ccc(-c3ccc(N(c4ccccc4)c4cccc5ccccc45)cc3)cc2)c2cccc3ccccc23)cc1 ; "NPB"]

A light emitting layer was thermally vacuum deposited thereon as follows. The light emitting layer was deposited by depositing a compound of 9-[4-(4,6-diphenyl-1,3,5-triazin-2-yl)phenyl]-9′-phenyl-3,3′-Bi-9H-carbazole as a host to have a thickness of 400 Å and doping the deposited layer with a green phosphorescent dopant Ir(ppy)₃ at 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10-8 to 10-6 torr for each material, and used for the manufacture of OLED.

### Examples 1 to 53 and Comparative Examples 2 to 5

Organic light emitting devices were manufactured in the same manner as in Experimental Example 1, except that Compounds shown in the following Table 8 were used instead of the compound NPB used when the hole transport layer was formed in Experimental Example 1, and the driving voltage and light emitting efficiency of the organic electroluminescence device according to Experimental Example 1 are shown in the following Table 8.

In this case, the hole transport compounds of the Comparative Examples except for NPB are shown as follows.

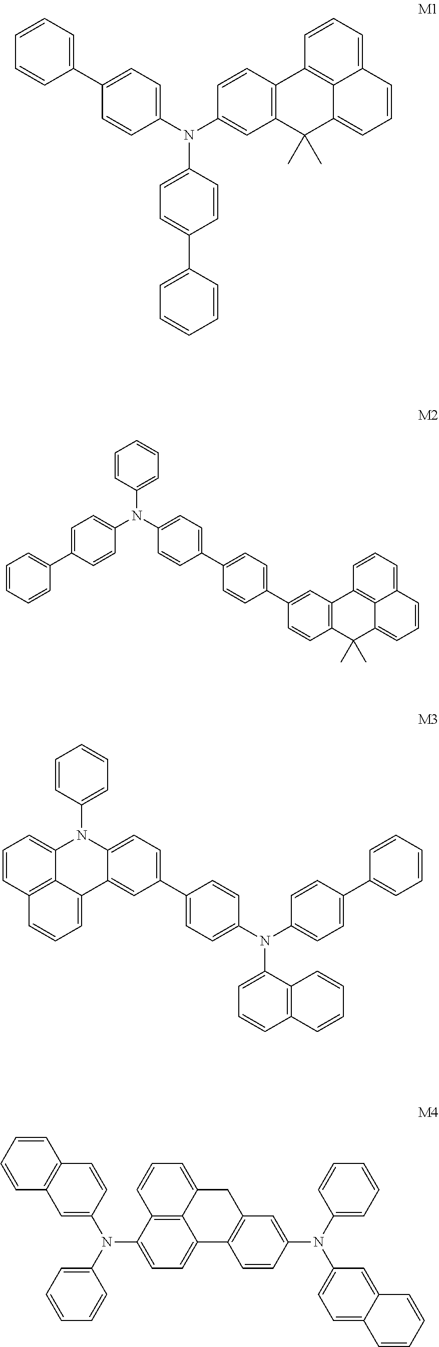 [SMILES: CC1(C)c2cc(N(c3ccc(-c4ccccc4)cc3)c3ccc(-c4ccccc4)cc3)ccc2-c2cccc3cccc1c23 ; CC1(C)c2ccc(-c3ccc(-c4ccc(N(c5ccccc5)c5ccc(-c6ccccc6)cc5)cc4)cc3)cc2-c2cccc3cccc1c23 ; c1ccc(-c2ccc(N(c3ccc(-c4ccc5c(c4)-c4cccc6cccc(c46)N5c4ccccc4)cc3)c3cccc4ccccc34)cc2)cc1 ; c1ccc(N(c2ccc3c(c2)Cc2cccc4c(N(c5ccccc5)c5ccc6ccccc6c5)ccc-3c24)c2ccc3ccccc3c2)cc1]

### (2) Driving Voltage and Light Emitting Efficiency of Organic Light Emitting Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

Characteristics of the organic electroluminescence device of the present invention are as shown in the following Table 8

TABLE 8

| | | | Light | |
|---|---|---|---|---|
| | | Driving | emitting | Service |
| | | voltage | efficiency | life |
| | Compound | (V) | (cd/A) | (T₉₀) |
| Example 1 | 002 | 4.15 | 120.46 | 153 |
| Example 2 | 003 | 4.11 | 118.53 | 168 |
| Example 3 | 007 | 3.97 | 123.99 | 165 |
| Example 4 | 008 | 4.09 | 125.37 | 156 |
| Example 5 | 016 | 3.87 | 125.11 | 144 |
| Example 6 | 017 | 4.09 | 120.54 | 171 |
| Example 7 | 019 | 4.01 | 118.32 | 172 |
| Example 8 | 020 | 4.14 | 119.76 | 156 |
| Example 9 | 026 | 3.98 | 120.12 | 169 |
| Example 10 | 029 | 4.09 | 121.23 | 145 |
| Example 11 | 045 | 4.01 | 122.95 | 172 |
| Example 12 | 054 | 4.14 | 124.34 | 152 |
| Example 13 | 059 | 3.99 | 123.52 | 161 |
| Example 14 | 077 | 3.91 | 120.55 | 155 |
| Example 15 | 091 | 3.98 | 124.17 | 178 |
| Example 16 | 103 | 4.17 | 124.81 | 180 |
| Example 17 | 105 | 4.09 | 125.53 | 163 |
| Example 18 | 106 | 4.01 | 123.10 | 167 |
| Example 19 | 116 | 3.97 | 122.34 | 154 |
| Example 20 | 119 | 4.13 | 119.98 | 142 |
| Example 21 | 122 | 4.15 | 116.79 | 155 |
| Example 22 | 131 | 4.04 | 119.86 | 149 |
| Example 23 | 132 | 3.88 | 121.44 | 166 |
| Example 24 | 133 | 4.02 | 124.79 | 161 |
| Example 25 | 145 | 4.13 | 119.64 | 165 |
| Example 26 | 158 | 4.13 | 123.63 | 157 |
| Example 27 | 160 | 4.05 | 123.71 | 163 |
| Example 28 | 170 | 3.95 | 120.79 | 172 |
| Example 29 | 182 | 3.93 | 119.97 | 171 |
| Example 30 | 183 | 4.11 | 125.65 | 160 |
| Example 31 | 197 | 3.87 | 120.46 | 172 |
| Example 32 | 199 | 4.17 | 123.31 | 168 |
| Example 33 | 207 | 4.04 | 121.54 | 158 |
| Example 34 | 223 | 4.10 | 127.81 | 167 |
| Example 35 | 232 | 3.87 | 119.82 | 152 |
| Example 36 | 253 | 4.11 | 123.76 | 171 |
| Example 37 | 302 | 3.90 | 121.04 | 152 |
| Example 38 | 319 | 4.01 | 122.76 | 176 |
| Example 39 | 332 | 4.06 | 123.30 | 166 |
| Example 40 | 333 | 3.83 | 123.35 | 157 |
| Example 41 | 346 | 4.11 | 124.41 | 142 |
| Example 42 | 353 | 4.12 | 123.76 | 155 |
| Example 43 | 367 | 3.97 | 120.78 | 173 |
| Example 44 | 368 | 3.93 | 122.77 | 170 |
| Example 45 | 381 | 4.14 | 120.09 | 154 |
| Example 46 | 391 | 3.87 | 124.30 | 167 |
| Example 47 | 422 | 3.84 | 120.74 | 152 |
| Example 48 | 429 | 4.17 | 123.30 | 160 |
| Example 49 | 451 | 4.04 | 120.36 | 157 |
| Example 50 | 479 | 4.10 | 123.34 | 166 |
| Example 51 | 480 | 3.87 | 120.35 | 140 |
| Example 52 | 486 | 4.11 | 122.41 | 149 |
| Example 53 | 502 | 4.12 | 118.35 | 202 |
| Comparative | NPB | 4.55 | 101.01 | 113 |
| Example 1 | | | | |
| Comparative | M1 | 4.41 | 110.55 | 134 |
| Example 2 | | | | |
| Comparative | M2 | 4.36 | 108.76 | 130 |
| Example 3 | | | | |
| Comparative | M3 | 4.87 | 107.54 | 125 |
| Example 4 | | | | |
| Comparative | M4 | 4.34 | 96.42 | 114 |
| Example 5 | | | | |

It could be seen that the devices of Examples 1 to 53 according to an exemplary embodiment of the present invention had a lower driving voltage and better efficiency and service life than the devices of Comparative Examples 1 to 5.

### Experimental Example 2

### (1) Manufacture of Organic Light Emitting Device

Trichloroethylene, acetone, ethanol, and distilled water were each sequentially used to ultrasonically wash a transparent electrode ITO thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris (N, N-(2-naphthyl)-phenylamino)triphenyl amine (2-TNATA) was placed in a cell in the vacuum deposition apparatus.

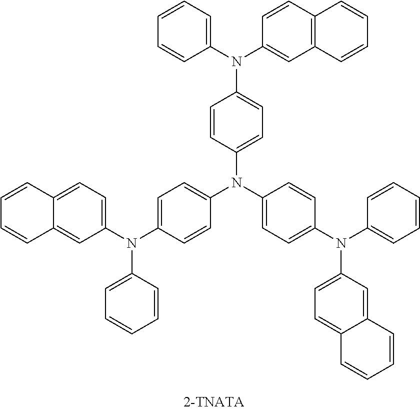 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10-6 torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N, N′-bis (α-naphthyl)-N, N′-diphenyl-4,4′-diamine (NPB) in another cell in the vacuum deposition apparatus and applying current to the cell to evaporate NPB.

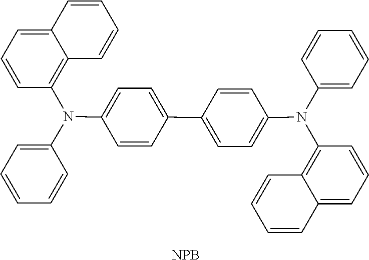 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as a light emitting layer was deposited thereon. Specifically, a blue light emitting host material H1 was vacuum deposited to have a thickness of 200 Å on one cell in the vacuum deposition apparatus, and a blue light emitting dopant material D1 was vacuum deposited thereon in an amount of 5% based on the host material.

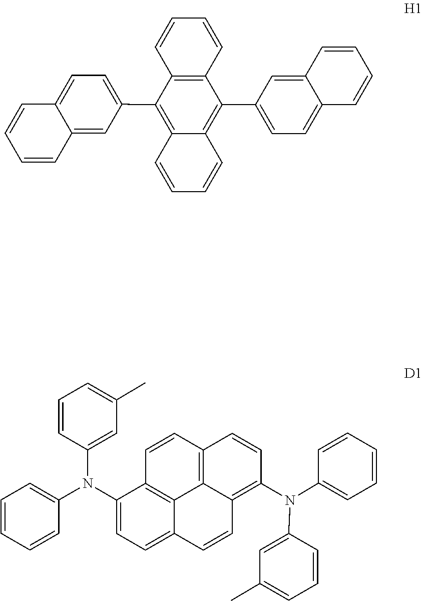

Subsequently, a compound having the following structural formula E1 as an electron transport layer was deposited to have a thickness of 300 Å.

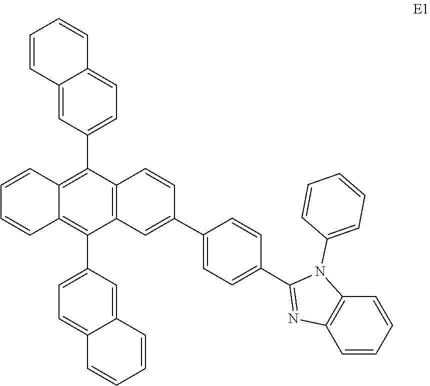 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10-8 to 10-6 torr for each material, and used for the manufacture of OLED.

An organic electroluminescence device was manufactured in the same manner as in Experimental Example 2, except that a hole transport layer NPB was formed to have a thickness of 150 Å, and then an electron blocking layer was formed to have a thickness of 50 Å on the upper portion of the hole transport layer using the compound shown in the following Table 9 in Experimental Example 2. The results of measuring the driving voltage, light emitting efficiency, and service life (T₉₅) of the blue organic light emitting device manufactured according to the present invention are shown as in the following Table 9. In this case, electron blocking layer compounds of the Comparative Examples are as follows.

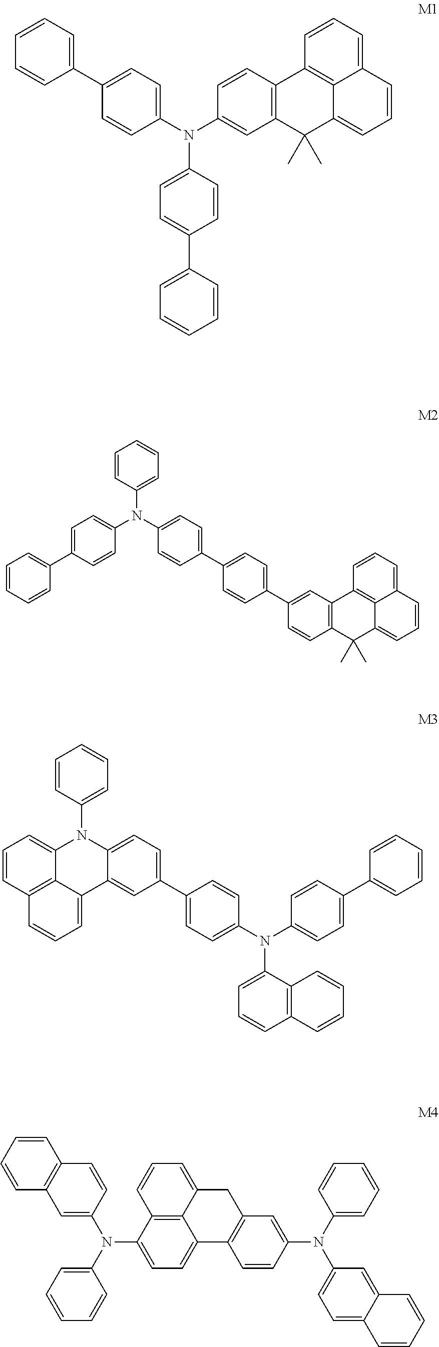 [SMILES: CC1(C)c2cc(N(c3ccc(-c4ccccc4)cc3)c3ccc(-c4ccccc4)cc3)ccc2-c2cccc3cccc1c23 ; CC1(C)c2ccc(-c3ccc(-c4ccc(N(c5ccccc5)c5ccc(-c6ccccc6)cc5)cc4)cc3)cc2-c2cccc3cccc1c23 ; c1ccc(-c2ccc(N(c3ccc(-c4ccc5c(c4)-c4cccc6cccc(c46)N5c4ccccc4)cc3)c3cccc4ccccc34)cc2)cc1 ; c1ccc(N(c2ccc3c(c2)Cc2cccc4c(N(c5ccccc5)c5ccc6ccccc6c5)ccc-3c24)c2ccc3ccccc3c2)cc1]

TABLE 9

| | | | Light | |
|---|---|---|---|---|
| | | Driving | emitting | Service |
| | | voltage | efficiency | life |
| | Compound | (V) | (cd/A) | (T₉₅) |
| Example 54 | 016 | 5.31 | 7.28 | 57 |
| Example 55 | 019 | 5.38 | 6.99 | 62 |
| Example 56 | 029 | 5.25 | 6.86 | 63 |
| Example 57 | 059 | 5.31 | 7.13 | 57 |
| Example 58 | 091 | 5.19 | 6.92 | 59 |
| Example 59 | 103 | 5.26 | 6.90 | 60 |
| Example 60 | 116 | 5.24 | 7.31 | 59 |
| Example 61 | 119 | 5.36 | 6.96 | 63 |
| Example 62 | 122 | 5.41 | 6.92 | 59 |
| Example 63 | 133 | 5.43 | 6.91 | 61 |
| Example 64 | 197 | 5.38 | 6.88 | 57 |
| Example 65 | 199 | 5.40 | 6.93 | 58 |
| Example 66 | 207 | 5.31 | 7.22 | 60 |
| Example 67 | 223 | 5.26 | 6.94 | 53 |
| Example 68 | 232 | 5.43 | 7.03 | 56 |
| Example 69 | 253 | 5.23 | 7.25 | 59 |
| Example 70 | 302 | 5.32 | 6.88 | 60 |
| Example 71 | 319 | 5.27 | 6.93 | 53 |
| Example 72 | 332 | 5.13 | 7.27 | 58 |
| Example 73 | 333 | 5.18 | 6.91 | 56 |
| Example 74 | 346 | 5.32 | 6.89 | 54 |
| Example 75 | 353 | 5.35 | 6.83 | 57 |
| Example 76 | 367 | 5.26 | 7.12 | 61 |
| Example 77 | 442 | 5.18 | 7.23 | 61 |
| Example 78 | 451 | 5.32 | 6.97 | 52 |
| Example 79 | 479 | 5.20 | 7.12 | 55 |
| Example 80 | 480 | 5.28 | 7.28 | 54 |
| Example 81 | 486 | 5.35 | 7.09 | 58 |
| Comparative | M1 | 5.62 | 6.41 | 47 |
| Example 6 | | | | |
| Comparative | M2 | 5.63 | 6.65 | 45 |
| Example 7 | | | | |
| Comparative | M3 | 5.96 | 6.58 | 43 |
| Example 8 | | | | |
| Comparative | M4 | 5.60 | 5.77 | 48 |
| Example 9 | | | | |

### Experimental Example 3

### 1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

The hole injection layer 4,4′,4″-tris[2-naphthyl (phenyl)amino] triphenylamine (2-TNATA) and the hole transport layer N, N′-diphenyl-(1,1′-biphenyl)-4,4′-diamine (NPB), which are common layers, were formed on the ITO transparent electrode (positive electrode).

A light emitting layer was thermally vacuum deposited thereon as follows. The light emitting layer was deposited to have a thickness of 500 Å by using a method for depositing two host compounds from one supply source by using an n-Host (n-type host) having a good electron transport capability among the compounds described in the following Table 10 as a single host or a first host and using a p-Host (p-type host) having a good hole transport capability as a second host, and doping the host with (piq)₂(Ir) (acac) at 3% relative to the weight of the host material using (piq)₂(Ir) (acac) as a red phosphorescent dopant, or doping the host with Ir (ppy)₃ at 7% relative to the weight of the host material using Ir (ppy)₃ as a green phosphorescent dopant.

Thereafter, BCP was deposited as a hole blocking layer to have a thickness of 60 Å, and Alq₃ was deposited as an electron transport layer to have a thickness of 200 Å thereon.

In this case, when two hosts are used, the compounds used as n-Host are as follows.

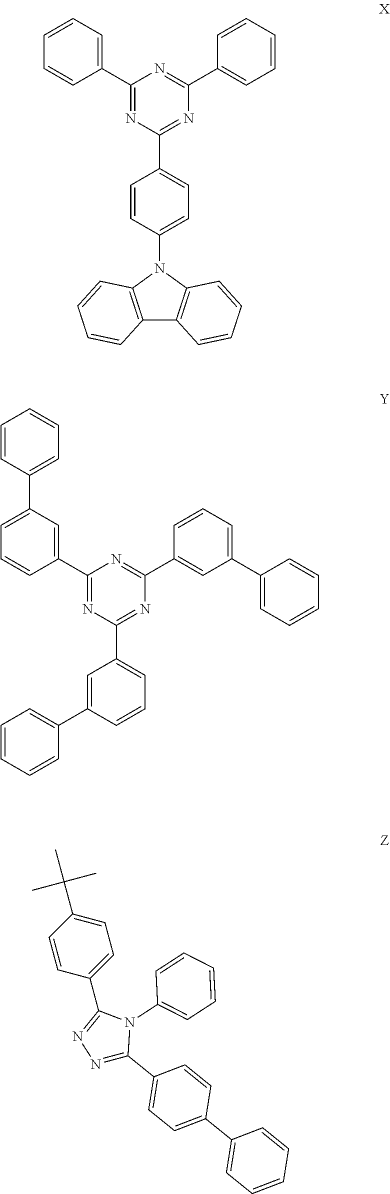 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

Finally, an organic light emitting device was manufactured by depositing lithium fluoride (LiF) to have a thickness of 10 Å on the electron transport layer to form an electron injection layer, and then depositing an aluminum (Al) negative electrode to have a thickness of 1,200 Å on the electron injection layer to form a negative electrode.

Specifically, the compounds used as hosts in Examples 82 to 106 and Comparative Examples 10 to 14 are shown as in the following Table 10.

In this case, Compounds M1 and M2 used as hosts in Comparative Examples 10 to 14 of the following Table 10 are as follows.

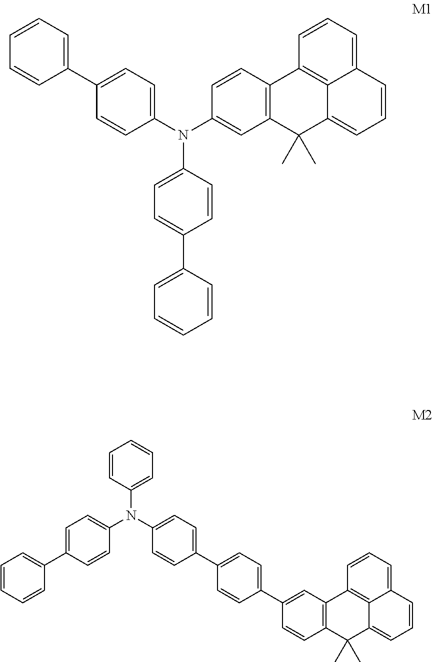 [SMILES: CC1(C)c2cc(N(c3ccc(-c4ccccc4)cc3)c3ccc(-c4ccccc4)cc3)ccc2-c2cccc3cccc1c23 ; CC1(C)c2ccc(-c3ccc(-c4ccc(N(c5ccccc5)c5ccc(-c6ccccc6)cc5)cc4)cc3)cc2-c2cccc3cccc1c23]

Meanwhile, all the organic compounds required for manufacturing an organic light emitting device were subjected to vacuum sublimed purification under 10-8 to 10-6 torr for each material, and used for the manufacture of the organic light emitting device.

### 2) Driving Voltage and Light Emitting Efficiency of Organic Light Emitting Device

For the organic electroluminescence device manufactured as described above, electroluminescence (EL) characteristics were measured by M7000 manufactured by McScience Inc., and based on the measurement result thereof, T95 was measured by a service life measurement device (M6000) manufactured by McScience Inc., when the reference luminance was 6,000 cd/m². The results of measuring the driving voltage, light emitting efficiency, light emission color and service life of the organic light emitting device manufactured according to the present invention are shown in the following Table 10.

TABLE 10

| | | | Driving | | Light | Service |
|---|---|---|---|---|---|---|
| | First | Second | voltage | Efficiency | emission | life |
| | host | host | (V) | (cd/A) | color | (T₉₅) |
| Example 82 | 103 | | 4.05 | 27.7 | Red | 76 |
| Example 83 | | | 4.08 | 67.6 | Green | 78 |
| Example 84 | 116 | | 4.09 | 26.4 | Red | 69 |
| Example 85 | | | 4.12 | 74.2 | Green | 85 |
| Example 86 | 119 | | 3.96 | 28.7 | Red | 84 |
| Example 87 | | | 4.19 | 69.4 | Green | 92 |
| Example 88 | 319 | | 3.87 | 29.3 | Red | 92 |
| Example 89 | | | 3.94 | 71.6 | Green | 101 |
| Example 90 | 333 | | 3.85 | 26.5 | Red | 87 |
| Example 91 | | | 4.06 | 73.0 | Green | 83 |
| Example 92 | X | 103 | 3.67 | 45.1 | Red | 155 |
| Example 93 | | 116 | 3.71 | 43.4 | Red | 172 |
| Example 94 | | 119 | 3.82 | 41.8 | Red | 144 |
| Example 95 | | 319 | 3.87 | 39.5 | Red | 157 |
| Example 96 | | 333 | 3.78 | 42.6 | Red | 148 |
| Example 97 | Y | 103 | 3.73 | 110.3 | Green | 158 |
| Example 98 | | 116 | 3.91 | 104.5 | Green | 162 |
| Example 99 | | 119 | 3.84 | 113.8 | Green | 166 |
| Example 100 | | 319 | 3.88 | 110.2 | Green | 175 |
| Example 101 | | 333 | 4.01 | 106.1 | Green | 165 |
| Example 102 | Z | 103 | 3.95 | 42.6 | Red | 159 |
| Example 103 | | 116 | 3.87 | 40.2 | Red | 146 |
| Example 104 | | 119 | 3.80 | 41.3 | Red | 147 |
| Example 105 | | 319 | 3.92 | 44.9 | Red | 149 |
| Example 106 | | 333 | 3.89 | 41.3 | Red | 153 |
| Comparative | M1 | | 4.43 | 21.6 | Red | 53 |
| Example 10 | | | | | | |
| Comparative | X | M1 | 4.22 | 34.5 | Red | 110 |
| Example 11 | | | | | | |
| Comparative | M2 | | 4.54 | 63.7 | Green | 65 |
| Example 12 | | | | | | |
| Comparative | Y | M2 | 4.35 | 89.5 | Green | 135 |
| Example 13 | | | | | | |
| Comparative | Z | M1 | 4.29 | 33.3 | Red | 109 |
| Example 14 | | | | | | |

From Experimental Example 3, it could be confirmed that in the case of the organic light emitting devices of Examples 82 to 91 in which a light emitting layer was formed using the compound according to the present application as a single host material, the light emitting efficiency and service life were better than the organic light emitting devices of Comparative Examples 10 and 12 in which the compound according to the present application was not used as a single host material when a light emitting layer was formed using the compound according to the present application as a single host material.

Further, from Experimental Example 3, it could be confirmed that in the case of the organic light emitting devices of Examples 92 to 106 in which a light emitting layer was formed simultaneously using a first host material corresponding to n-Host and the compound according to the present application as a second host material corresponding to p-Host, the light emitting efficiency and service life were better than the organic light emitting devices of Comparative Examples 11, 13 and 14 in which a light emitting layer was formed simultaneously using the first host material corresponding to n-Host and a compound other than the compound according to the present application as a second host material corresponding to p-Host.

In addition, it could be confirmed that the light emitting efficiency and service life of the organic light emitting devices of Examples 82 to 91 in which a light emitting layer was formed using the compound according to the present application as a single host material are similar to or better than the organic light emitting devices of Comparative Examples 11, 13 and 14 in which a light emitting layer was formed simultaneously using a first host material corresponding to n-Host and a compound other than the compound according to the present application as a second host material corresponding to p-Host.

This generally means that the light emitting efficiency and service life of the organic light emitting device could be remarkably improved when the compound according to the present application was used as a host material considering that the case where n-Host (n-type host) having a good electron transport capability was used as a first host and p-Host (p-type host) having a good hole transport capability was used as a second host has better light emitting efficiency and service life than an organic light emitting device manufactured using a single host material.

It is judged that this is because holes and electrons can be efficiently injected into the light emitting layer from each charge transfer layer when the compound according to the present application is used as a host material. It is judged that this is because such a point is also affected by the orientation and the size of the space formed by the interaction of materials during deposition as described above.

It is judged that this is because the efficient injection of holes and electrons into the light emitting layer is also affected by the orientation and the size of the space formed by the interaction of materials during deposition, and the efficient injection of holes and electrons into the light emitting layer is an effect caused by the difference in orientation characteristics and size of the space of the compound of the present application and M1 and M2 as described above.

The present invention is not limited to the Examples, but may be prepared in various forms, and a person with ordinary skill in the art to which the present invention belongs will understand that the present invention can be implemented in another specific form without changing the technical spirit or essential feature of the present invention. Therefore, it should be understood that the above-described Examples are illustrative only in all aspects and are not restrictive.

1. A heterocyclic compound represented by the following Chemical Formula 1: 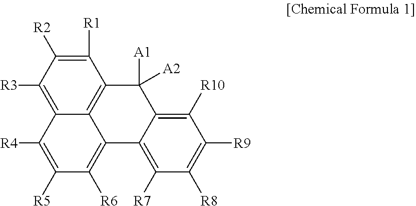
in Chemical Formula 1,
A1 and A2 are the same as or different from each other, and are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
R1 to R5 and R7 to R10 are the same as or different from each other, and are each independently selected from the group consisting of hydrogen; deuterium; a halogen group; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; SiRR′R″; —P(═O)RR′; the following Chemical Formula 1-1 and the following Chemical Formula 1-2,
R6 is represented by the following Chemical Formula 1-1; or the following Chemical Formula 1-2, 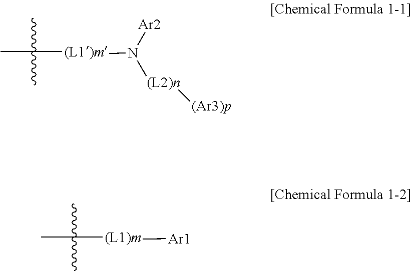
in Chemical Formulae 1-1 and 1-2,
L1, L1′ and L2 are the same as or different from each other, and are each independently a direct bond; a substituted or unsubstituted C6 to C60 arylene group; or a substituted or unsubstituted C2 to C60 heteroarylene group,
Ar1 to Ar3 are the same as or different from each other, and are each independently a substituted or unsubstituted C1 to C60 alkyl group; a substituted or unsubstituted C6 to C60 aryl group; or a substituted or unsubstituted C2 to C60 heteroaryl group,
m, m′ and n are each an integer from 0 to 3,
p is an integer from 1 to 5,
when m, m′, n and p are each 2 or higher, substituents in the parenthesis are the same as or different from each other, and
R, R′ and R″ are the same as or different from each other, and are each independently a substituted or unsubstituted C1 to C60 alkyl group; a substituted or unsubstituted C6 to C60 aryl group; or a substituted or unsubstituted C2 to C60 heteroaryl group.

2. The heterocyclic compound of claim 1, wherein Chemical Formula 1 is represented by any one of the following Chemical Formulae 2 to 4: 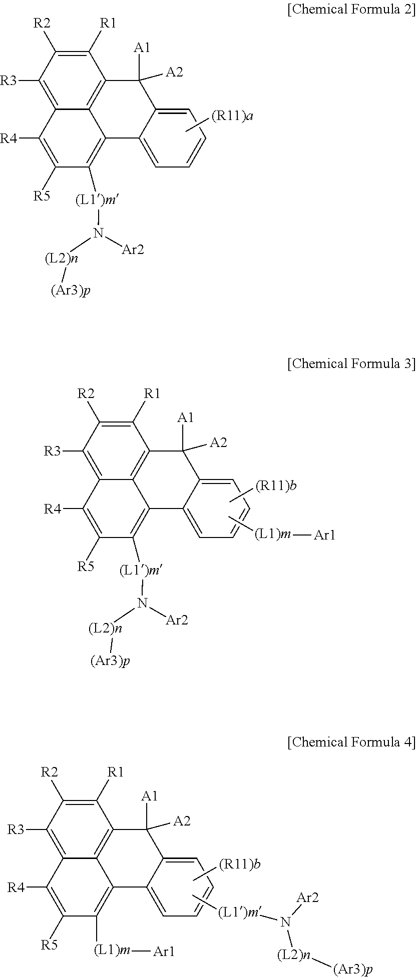
in Chemical Formulae 2 to 4,
the definitions of A1, A2, R1 to R5, L1, L1′, L2, Ar1 to Ar3, m, m′, n and p are the same as those in Chemical Formula 1,
R11 is hydrogen; or deuterium, and
a is an integer from 0 to 4, b is an integer from 0 to 3, and when a and b are each an integer of 2 or higher, substituents in the parenthesis are the same as or different from each other.

3. The heterocyclic compound of claim 1, wherein Ar1 is represented by any one of the following structural formulae: 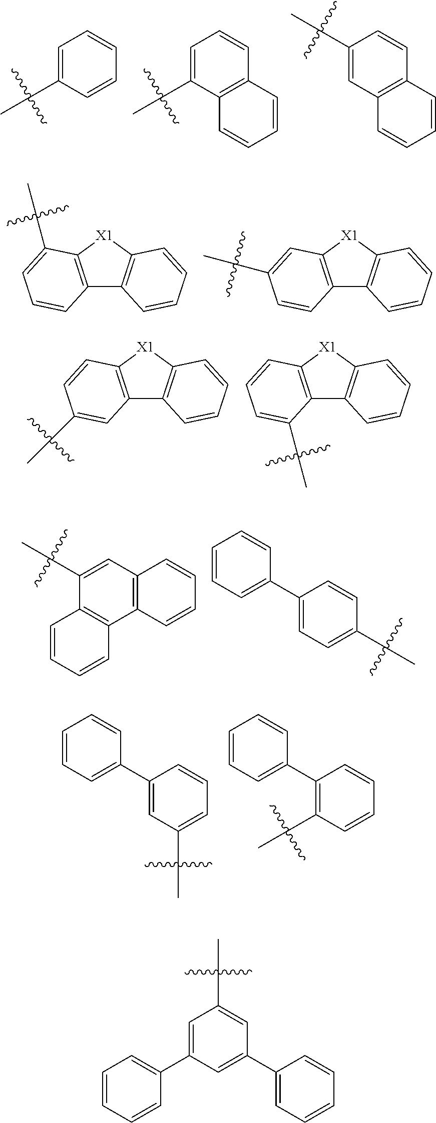
in the structural formulae,
X1 is O; S; or CRaRb,
Ra and Rb are the same as or different from each other, and are each independently a substituted or unsubstituted C1 to C60 alkyl group; or a substituted or unsubstituted C6 to C60 aryl group, and
the deuterium content of the structural formula is 0% or 100%.

4. The heterocyclic compound of claim 1, wherein the deuterium content of the heterocyclic compound of Chemical Formula 1 is 0% to 100%.

5. The heterocyclic compound of claim 1, wherein Chemical Formula 1 is represented by any one of the following compounds:

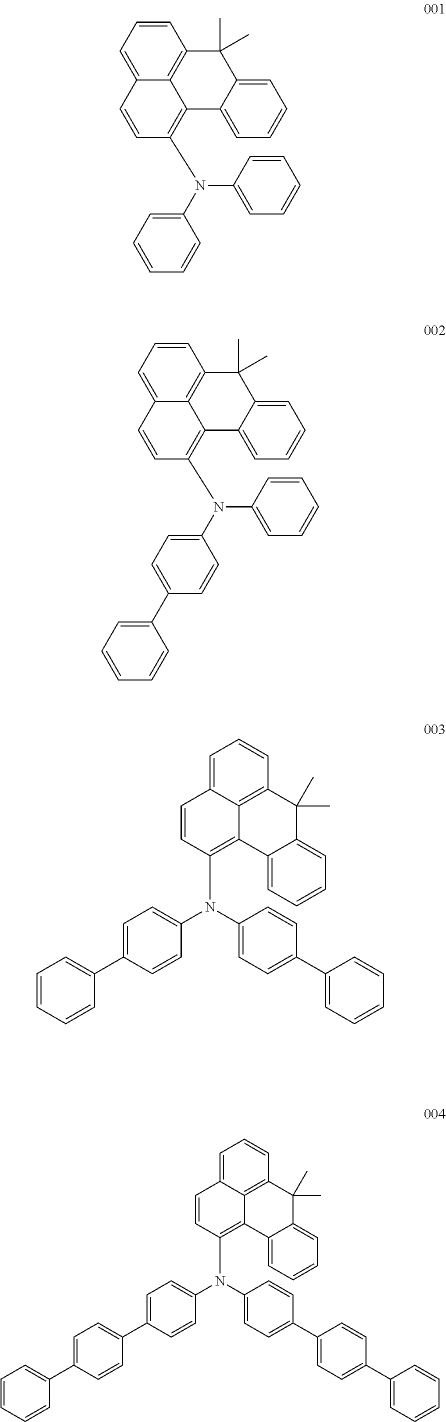 [SMILES: CC1(C)c2ccccc2-c2c(N(c3ccccc3)c3ccccc3)ccc3cccc1c23 ; CC1(C)c2ccccc2-c2c(N(c3ccccc3)c3ccc(-c4ccccc4)cc3)ccc3cccc1c23 ; CC1(C)c2ccccc2-c2c(N(c3ccc(-c4ccccc4)cc3)c3ccc(-c4ccccc4)cc3)ccc3cccc1c23 ; CC1(C)c2ccccc2-c2c(N(c3ccc(-c4ccc(-c5ccccc5)cc4)cc3)c3ccc(-c4ccc(-c5ccccc5)cc4)cc3)ccc3cccc1c23]

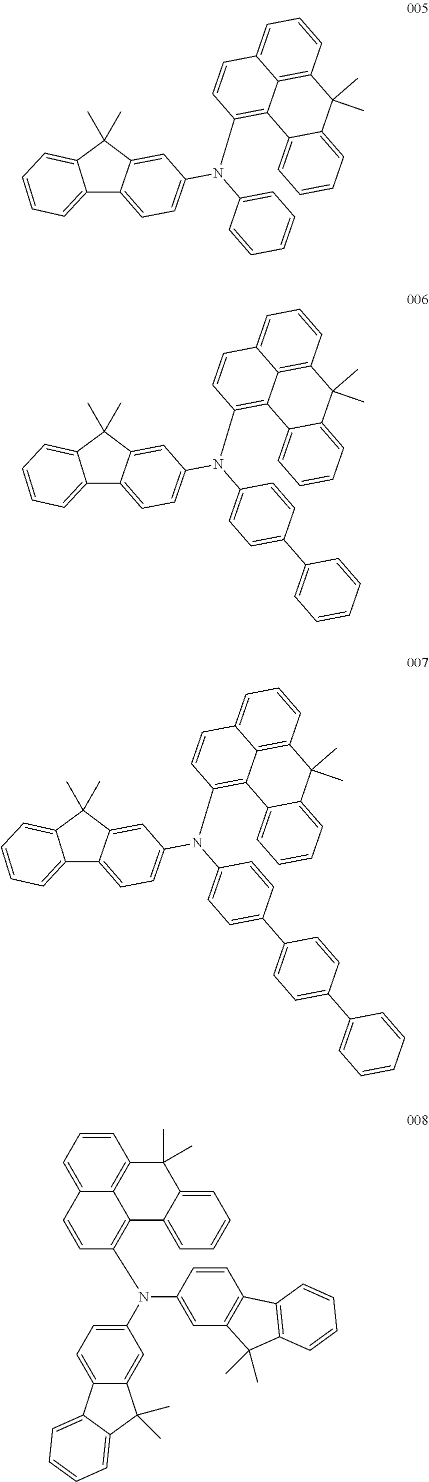 [SMILES: CC1(C)c2ccccc2-c2ccc(N(c3ccccc3)c3ccc4cccc5c4c3-c3ccccc3C5(C)C)cc21 ; CC1(C)c2ccccc2-c2ccc(N(c3ccc(-c4ccccc4)cc3)c3ccc4cccc5c4c3-c3ccccc3C5(C)C)cc21 ; CC1(C)c2ccccc2-c2ccc(N(c3ccc(-c4ccc(-c5ccccc5)cc4)cc3)c3ccc4cccc5c4c3-c3ccccc3C5(C)C)cc21 ; CC1(C)c2ccccc2-c2ccc(N(c3ccc4c(c3)C(C)(C)c3ccccc3-4)c3ccc4cccc5c4c3-c3ccccc3C5(C)C)cc21]

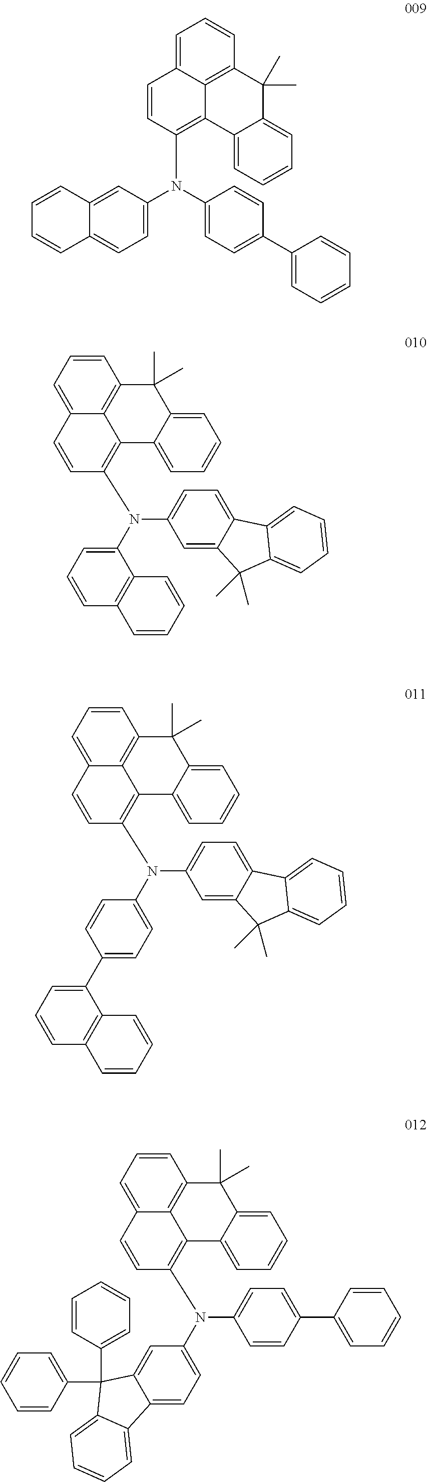 [SMILES: CC1(C)c2ccccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccccc3C5(C)C)c3cccc4ccccc34)cc21 ; CC1(C)c2ccccc2-c2ccc(N(c3ccc(-c4cccc5ccccc45)cc3)c3ccc4cccc5c4c3-c3ccccc3C5(C)C)cc21 ; CC1(C)c2ccccc2-c2c(N(c3ccc(-c4ccccc4)cc3)c3ccc4c(c3)C(c3ccccc3)(c3ccccc3)c3ccccc3-4)ccc3cccc1c23]

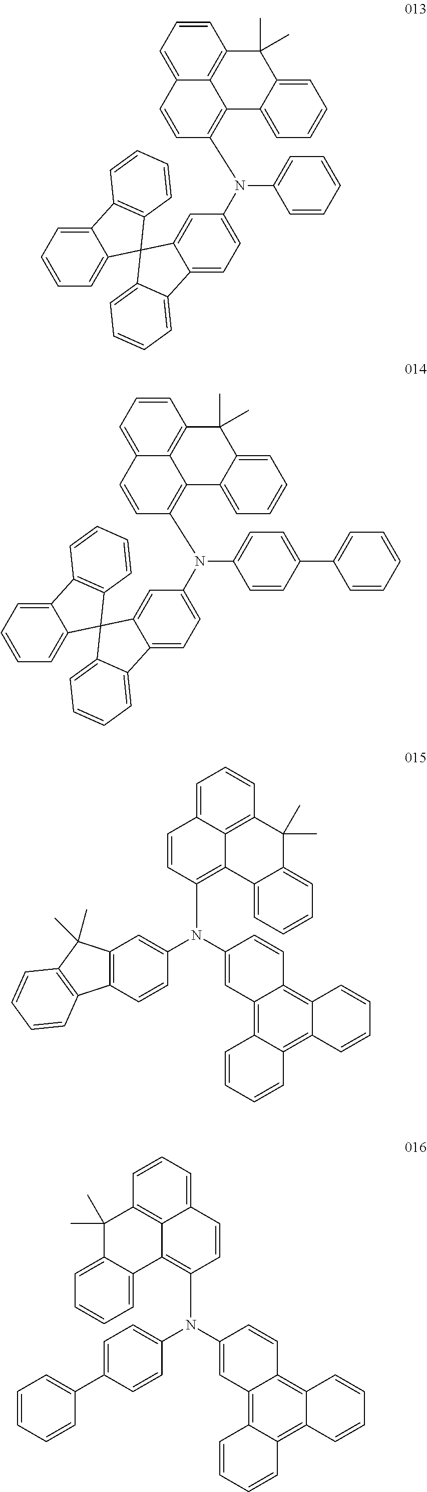 [SMILES: CC1(C)c2ccccc2-c2c(N(c3ccccc3)c3ccc4c(c3)C3(c5ccccc5-c5ccccc53)c3ccccc3-4)ccc3cccc1c23 ; CC1(C)c2ccccc2-c2c(N(c3ccc(-c4ccccc4)cc3)c3ccc4c(c3)C3(c5ccccc5-c5ccccc53)c3ccccc3-4)ccc3cccc1c23 ; CC1(C)c2ccccc2-c2ccc(N(c3ccc4c5ccccc5c5ccccc5c4c3)c3ccc4cccc5c4c3-c3ccccc3C5(C)C)cc21 ; CC1(C)c2ccccc2-c2c(N(c3ccc(-c4ccccc4)cc3)c3ccc4c5ccccc5c5ccccc5c4c3)ccc3cccc1c23]

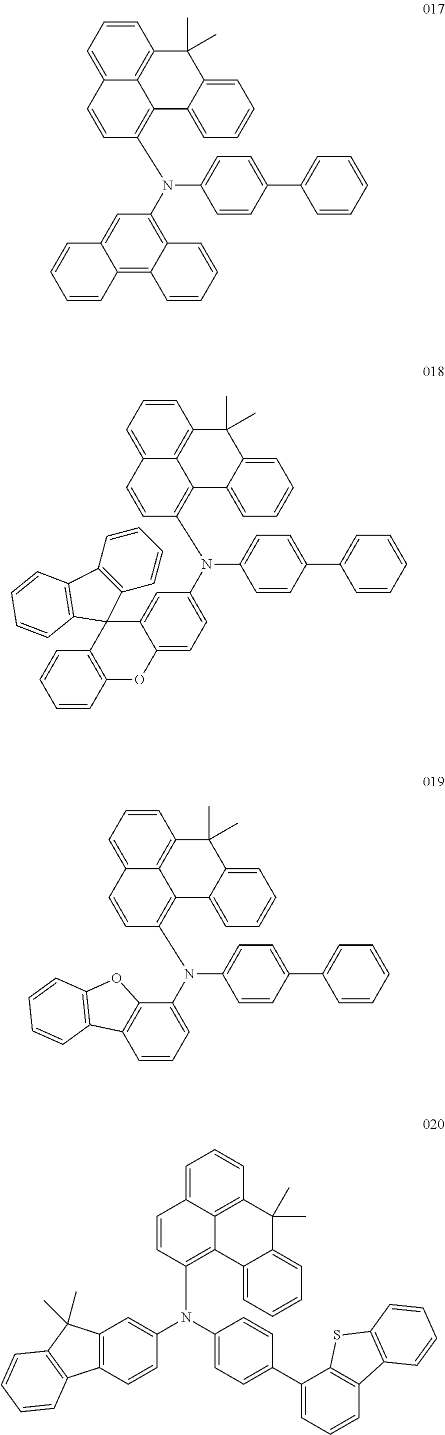 [SMILES: CC1(C)c2ccccc2-c2c(N(c3ccc(-c4ccccc4)cc3)c3cc4ccccc4c4ccccc34)ccc3cccc1c23 ; CC1(C)c2ccccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2ccccc2-c2c(N(c3ccc(-c4ccccc4)cc3)c3cccc4c3oc3ccccc34)ccc3cccc1c23 ; CC1(C)c2ccccc2-c2ccc(N(c3ccc(-c4cccc5c4sc4ccccc45)cc3)c3ccc4cccc5c4c3-c3ccccc3C5(C)C)cc21]

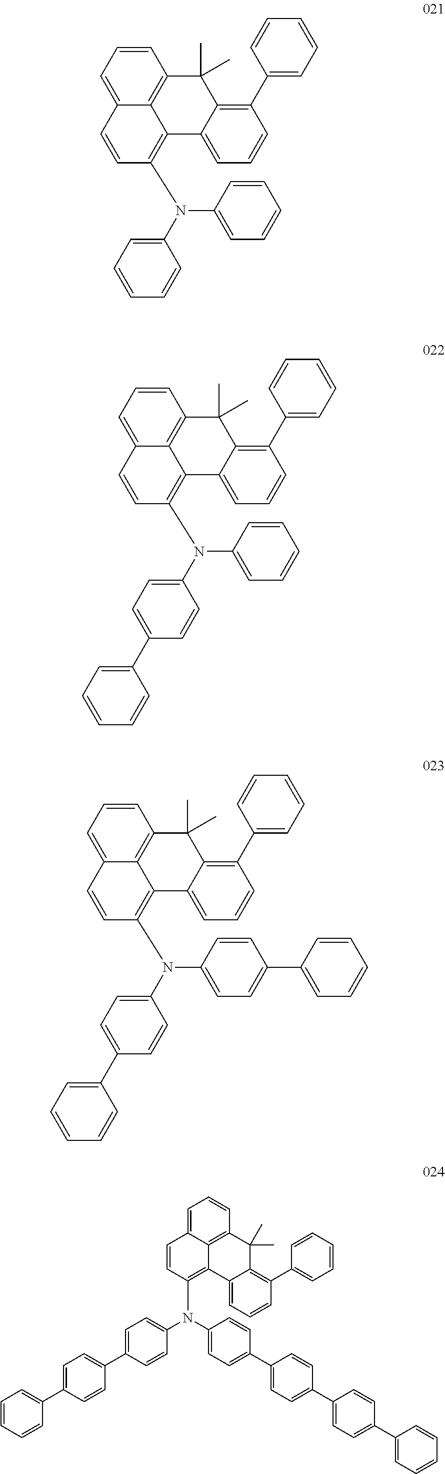 [SMILES: CC1(C)c2c(-c3ccccc3)cccc2-c2c(N(c3ccccc3)c3ccccc3)ccc3cccc1c23 ; CC1(C)c2c(-c3ccccc3)cccc2-c2c(N(c3ccccc3)c3ccc(-c4ccccc4)cc3)ccc3cccc1c23 ; CC1(C)c2c(-c3ccccc3)cccc2-c2c(N(c3ccc(-c4ccccc4)cc3)c3ccc(-c4ccccc4)cc3)ccc3cccc1c23 ; CC1(C)c2c(-c3ccccc3)cccc2-c2c(N(c3ccc(-c4ccc(-c5ccccc5)cc4)cc3)c3ccc(-c4ccc(-c5ccc(-c6ccccc6)cc5)cc4)cc3)ccc3cccc1c23]

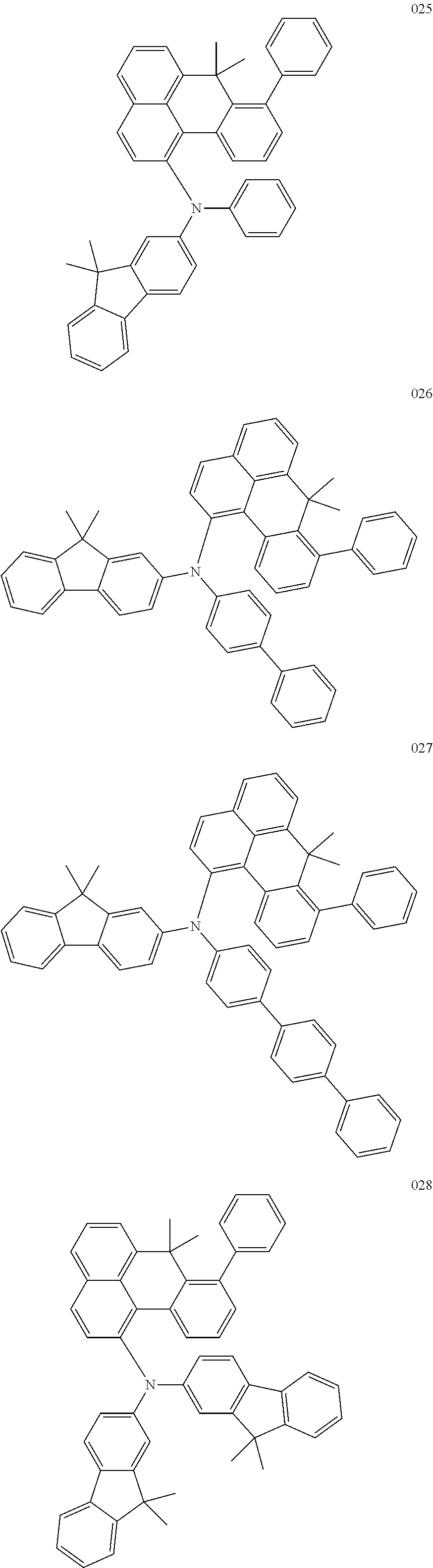 [SMILES: CC1(C)c2ccccc2-c2ccc(N(c3ccccc3)c3ccc4cccc5c4c3-c3cccc(-c4ccccc4)c3C5(C)C)cc21 ; CC1(C)c2ccccc2-c2ccc(N(c3ccc(-c4ccccc4)cc3)c3ccc4cccc5c4c3-c3cccc(-c4ccccc4)c3C5(C)C)cc21 ; CC1(C)c2ccccc2-c2ccc(N(c3ccc(-c4ccc(-c5ccccc5)cc4)cc3)c3ccc4cccc5c4c3-c3cccc(-c4ccccc4)c3C5(C)C)cc21 ; CC1(C)c2ccccc2-c2ccc(N(c3ccc4c(c3)C(C)(C)c3ccccc3-4)c3ccc4cccc5c4c3-c3cccc(-c4ccccc4)c3C5(C)C)cc21]

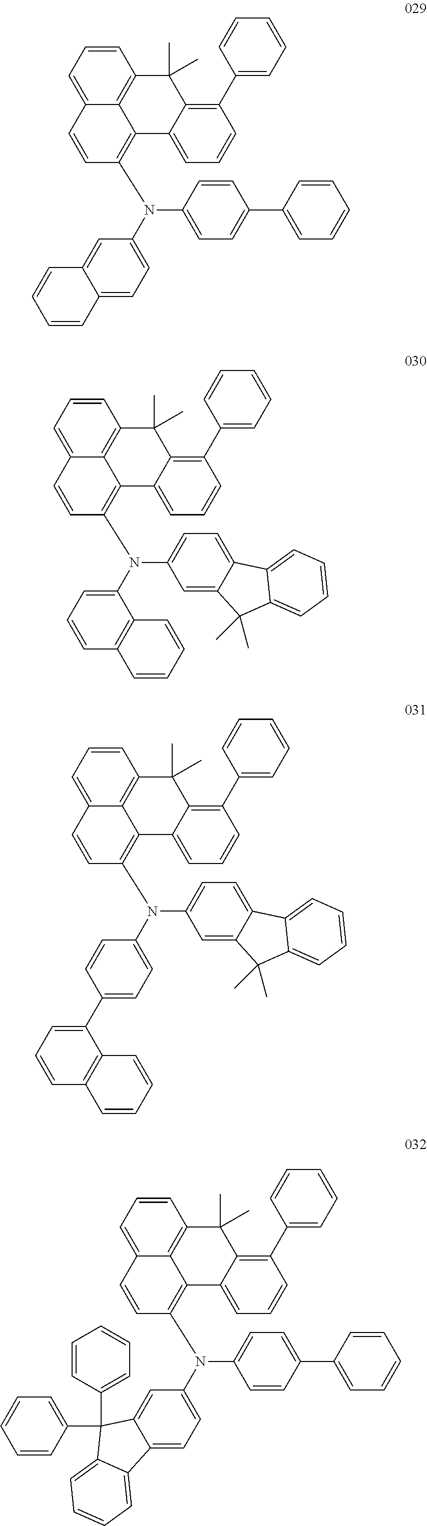 [SMILES: CC1(C)c2c(-c3ccccc3)cccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4ccccc4)c3C5(C)C)c3cccc4ccccc34)cc21 ; CC1(C)c2ccccc2-c2ccc(N(c3ccc(-c4cccc5ccccc45)cc3)c3ccc4cccc5c4c3-c3cccc(-c4ccccc4)c3C5(C)C)cc21 ; CC1(C)c2c(-c3ccccc3)cccc2-c2c(N(c3ccc(-c4ccccc4)cc3)c3ccc4c(c3)C(c3ccccc3)(c3ccccc3)c3ccccc3-4)ccc3cccc1c23]

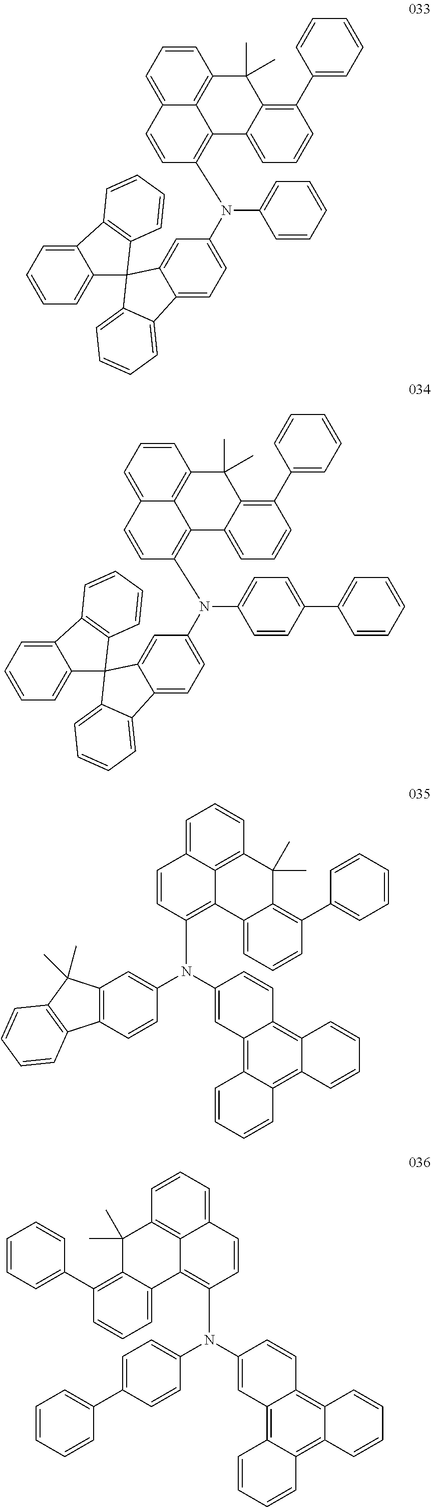 [SMILES: CC1(C)c2c(-c3ccccc3)cccc2-c2c(N(c3ccccc3)c3ccc4c(c3)C3(c5ccccc5-c5ccccc53)c3ccccc3-4)ccc3cccc1c23 ; CC1(C)c2c(-c3ccccc3)cccc2-c2c(N(c3ccc(-c4ccccc4)cc3)c3ccc4c(c3)C3(c5ccccc5-c5ccccc53)c3ccccc3-4)ccc3cccc1c23 ; CC1(C)c2ccccc2-c2ccc(N(c3ccc4c5ccccc5c5ccccc5c4c3)c3ccc4cccc5c4c3-c3cccc(-c4ccccc4)c3C5(C)C)cc21 ; CC1(C)c2c(-c3ccccc3)cccc2-c2c(N(c3ccc(-c4ccccc4)cc3)c3ccc4c5ccccc5c5ccccc5c4c3)ccc3cccc1c23]

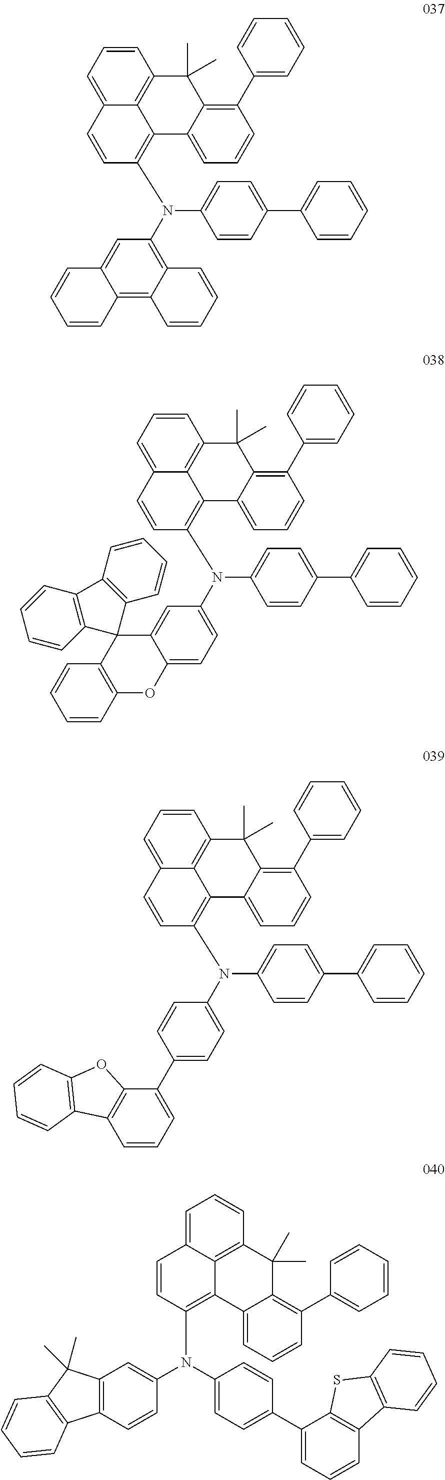 [SMILES: CC1(C)c2c(-c3ccccc3)cccc2-c2c(N(c3ccc(-c4ccccc4)cc3)c3cc4ccccc4c4ccccc34)ccc3cccc1c23 ; CC1(C)c2c(-c3ccccc3)cccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2c(-c3ccccc3)cccc2-c2c(N(c3ccc(-c4ccccc4)cc3)c3ccc(-c4cccc5c4oc4ccccc45)cc3)ccc3cccc1c23 ; CC1(C)c2ccccc2-c2ccc(N(c3ccc(-c4cccc5c4sc4ccccc45)cc3)c3ccc4cccc5c4c3-c3cccc(-c4ccccc4)c3C5(C)C)cc21]

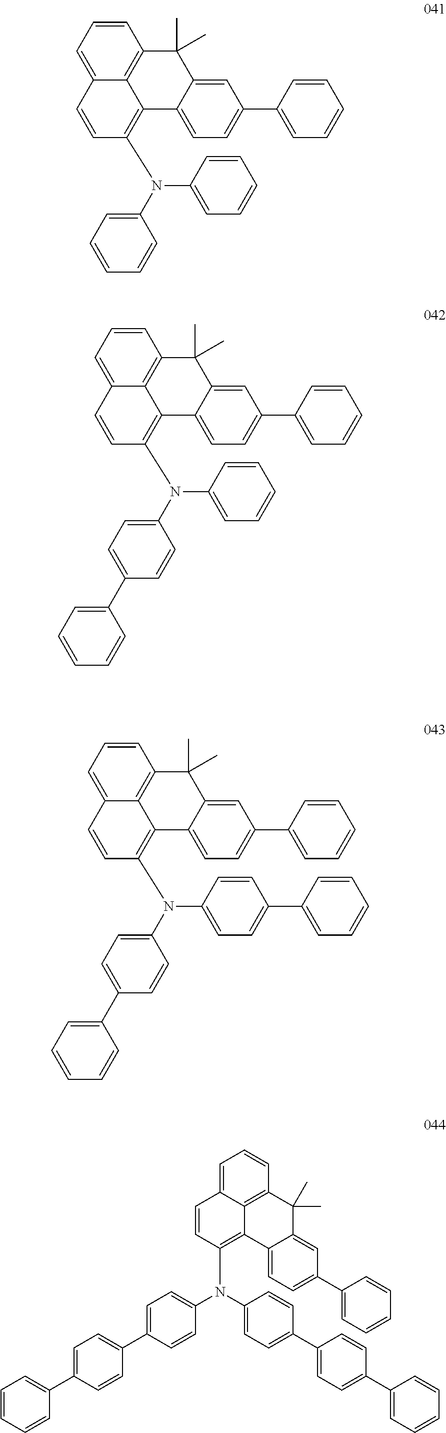 [SMILES: CC1(C)c2cc(-c3ccccc3)ccc2-c2c(N(c3ccccc3)c3ccccc3)ccc3cccc1c23 ; CC1(C)c2cc(-c3ccccc3)ccc2-c2c(N(c3ccccc3)c3ccc(-c4ccccc4)cc3)ccc3cccc1c23 ; CC1(C)c2cc(-c3ccccc3)ccc2-c2c(N(c3ccc(-c4ccccc4)cc3)c3ccc(-c4ccccc4)cc3)ccc3cccc1c23 ; CC1(C)c2cc(-c3ccccc3)ccc2-c2c(N(c3ccc(-c4ccc(-c5ccccc5)cc4)cc3)c3ccc(-c4ccc(-c5ccccc5)cc4)cc3)ccc3cccc1c23]

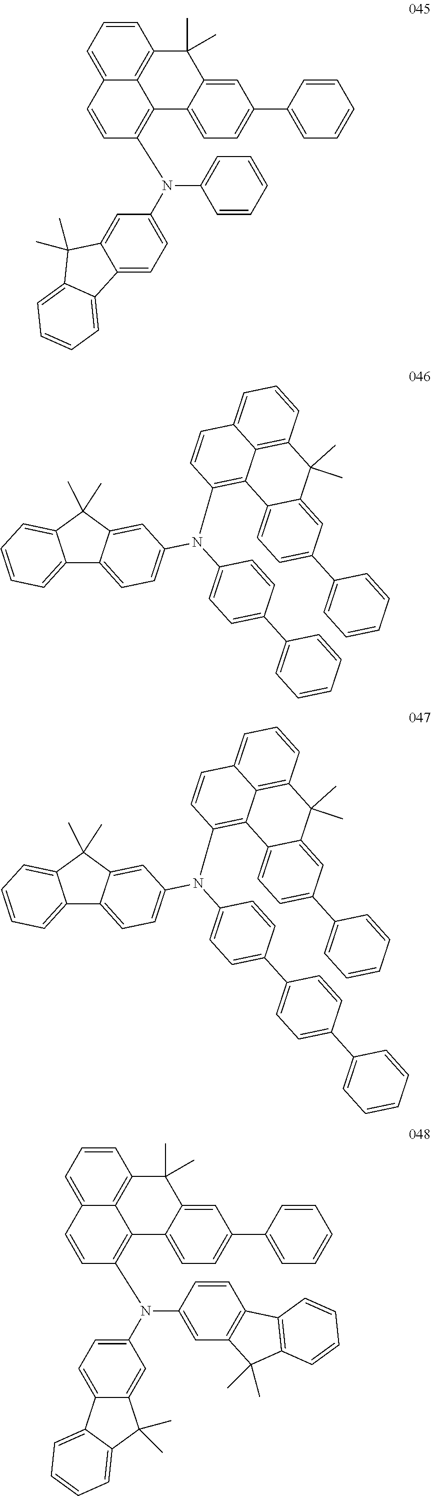

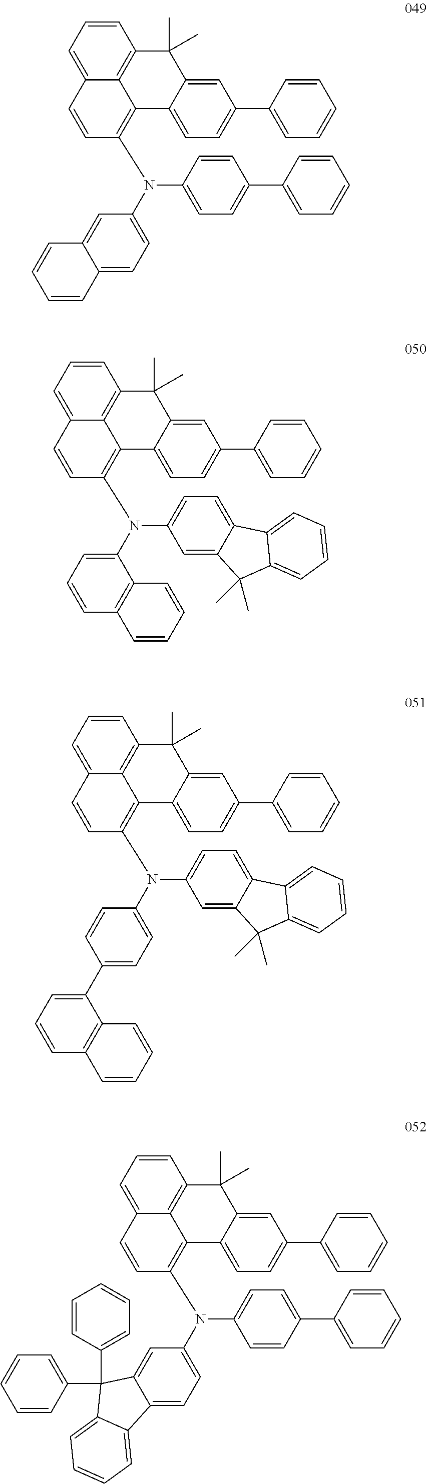 [SMILES: CC1(C)c2cc(-c3ccccc3)ccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccc(-c4ccccc4)cc3C5(C)C)c3cccc4ccccc34)cc21 ; CC1(C)c2ccccc2-c2ccc(N(c3ccc(-c4cccc5ccccc45)cc3)c3ccc4cccc5c4c3-c3ccc(-c4ccccc4)cc3C5(C)C)cc21 ; CC1(C)c2cc(-c3ccccc3)ccc2-c2c(N(c3ccc(-c4ccccc4)cc3)c3ccc4c(c3)C(c3ccccc3)(c3ccccc3)c3ccccc3-4)ccc3cccc1c23]

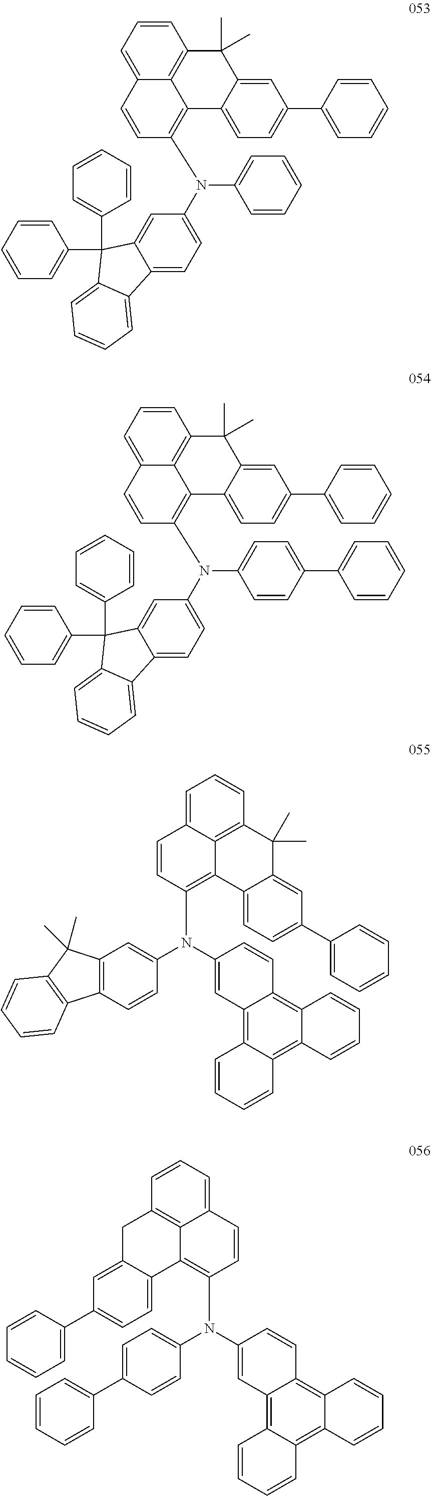 [SMILES: CC1(C)c2cc(-c3ccccc3)ccc2-c2c(N(c3ccccc3)c3ccc4c(c3)C(c3ccccc3)(c3ccccc3)c3ccccc3-4)ccc3cccc1c23 ; CC1(C)c2cc(-c3ccccc3)ccc2-c2c(N(c3ccc(-c4ccccc4)cc3)c3ccc4c(c3)C(c3ccccc3)(c3ccccc3)c3ccccc3-4)ccc3cccc1c23 ; CC1(C)c2ccccc2-c2ccc(N(c3ccc4c5ccccc5c5ccccc5c4c3)c3ccc4cccc5c4c3-c3ccc(-c4ccccc4)cc3C5(C)C)cc21 ; c1ccc(-c2ccc(N(c3ccc4c5ccccc5c5ccccc5c4c3)c3ccc4cccc5c4c3-c3ccc(-c4ccccc4)cc3C5)cc2)cc1]

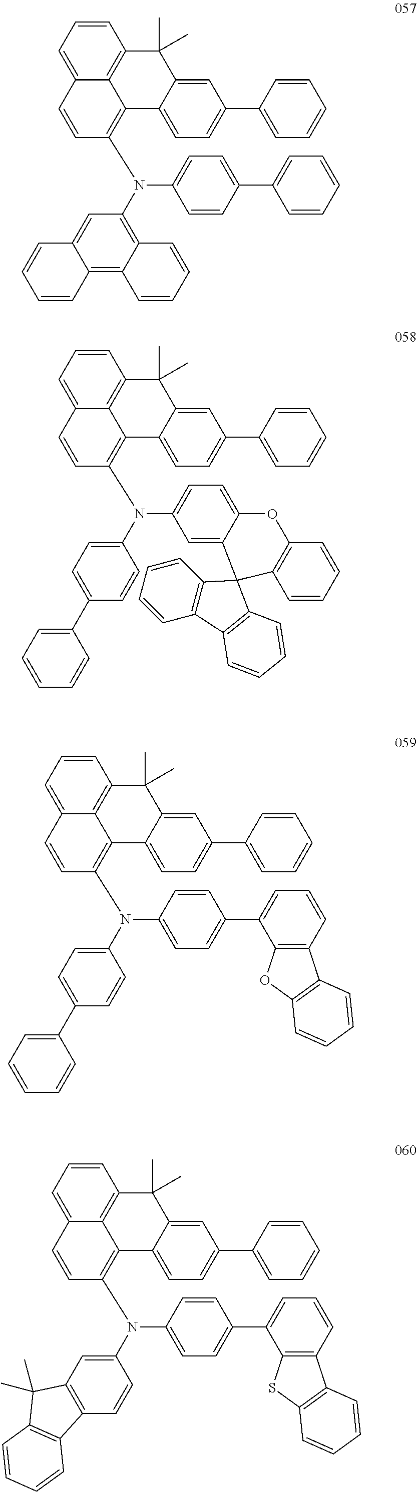 [SMILES: CC1(C)c2cc(-c3ccccc3)ccc2-c2c(N(c3ccc(-c4ccccc4)cc3)c3cc4ccccc4c4ccccc34)ccc3cccc1c23 ; CC1(C)c2cc(-c3ccccc3)ccc2-c2c(N(c3ccc(-c4ccccc4)cc3)c3ccc4c(c3)C3(c5ccccc5O4)c4ccccc4-c4ccccc43)ccc3cccc1c23 ; CC1(C)c2cc(-c3ccccc3)ccc2-c2c(N(c3ccc(-c4ccccc4)cc3)c3ccc(-c4cccc5c4oc4ccccc45)cc3)ccc3cccc1c23 ; CC1(C)c2ccccc2-c2ccc(N(c3ccc(-c4cccc5c4sc4ccccc45)cc3)c3ccc4cccc5c4c3-c3ccc(-c4ccccc4)cc3C5(C)C)cc21]

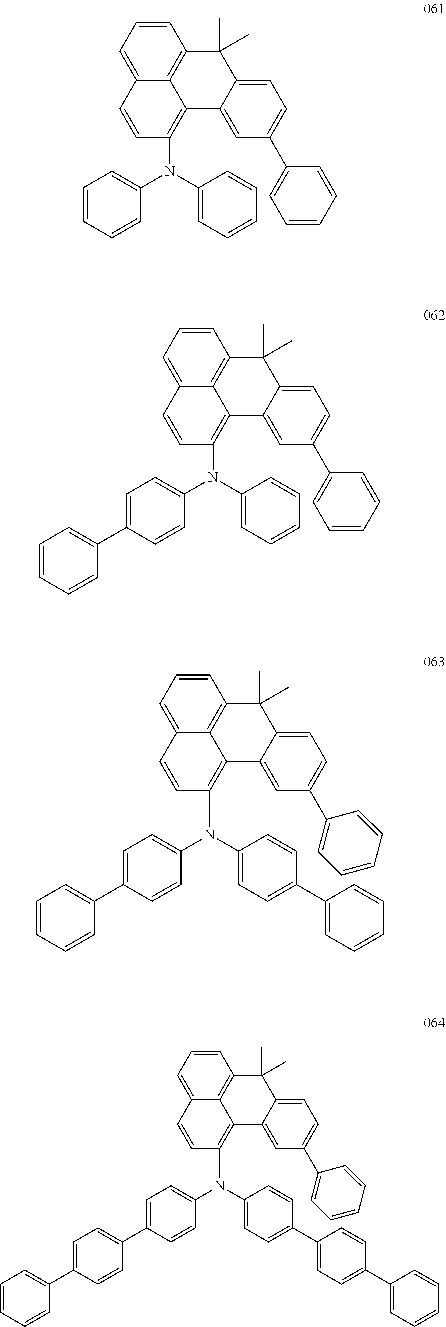 [SMILES: CC1(C)c2ccc(-c3ccccc3)cc2-c2c(N(c3ccccc3)c3ccccc3)ccc3cccc1c23 ; CC1(C)c2ccc(-c3ccccc3)cc2-c2c(N(c3ccccc3)c3ccc(-c4ccccc4)cc3)ccc3cccc1c23 ; CC1(C)c2ccc(-c3ccccc3)cc2-c2c(N(c3ccc(-c4ccccc4)cc3)c3ccc(-c4ccccc4)cc3)ccc3cccc1c23 ; CC1(C)c2ccc(-c3ccccc3)cc2-c2c(N(c3ccc(-c4ccc(-c5ccccc5)cc4)cc3)c3ccc(-c4ccc(-c5ccccc5)cc4)cc3)ccc3cccc1c23]

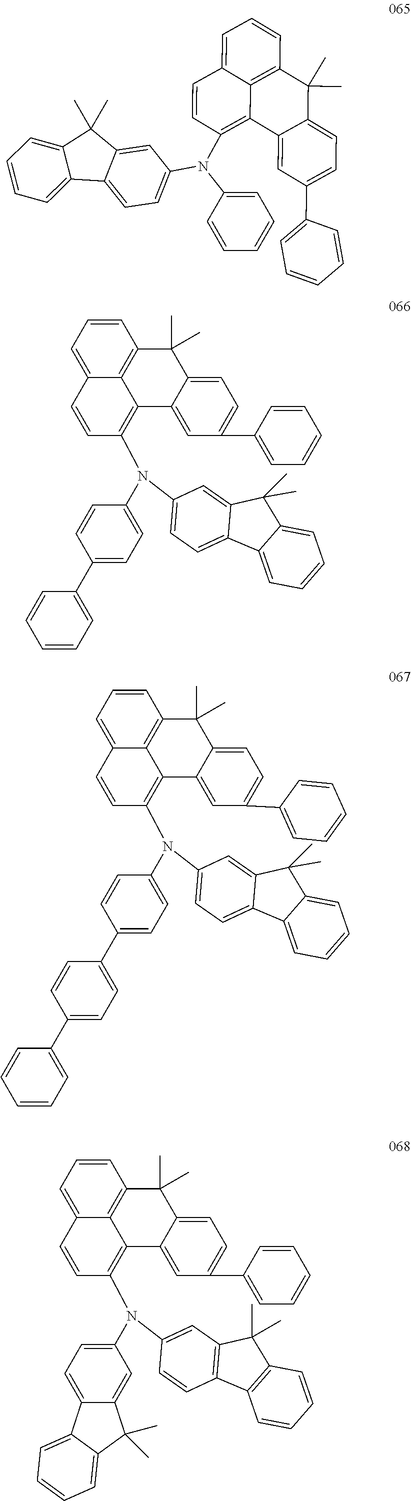 [SMILES: CC1(C)c2ccccc2-c2ccc(N(c3ccccc3)c3ccc4cccc5c4c3-c3cc(-c4ccccc4)ccc3C5(C)C)cc21 ; CC1(C)c2ccccc2-c2ccc(N(c3ccc(-c4ccccc4)cc3)c3ccc4cccc5c4c3-c3cc(-c4ccccc4)ccc3C5(C)C)cc21 ; CC1(C)c2ccccc2-c2ccc(N(c3ccc(-c4ccc(-c5ccccc5)cc4)cc3)c3ccc4cccc5c4c3-c3cc(-c4ccccc4)ccc3C5(C)C)cc21 ; CC1(C)c2ccccc2-c2ccc(N(c3ccc4c(c3)C(C)(C)c3ccccc3-4)c3ccc4cccc5c4c3-c3cc(-c4ccccc4)ccc3C5(C)C)cc21]

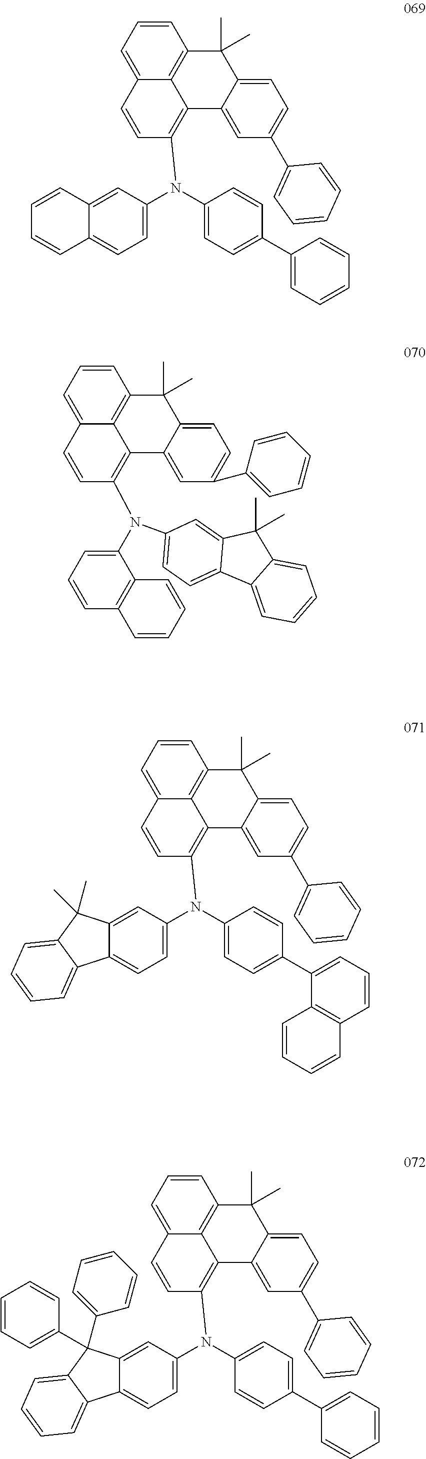 [SMILES: CC1(C)c2ccc(-c3ccccc3)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cc(-c4ccccc4)ccc3C5(C)C)c3cccc4ccccc34)cc21 ; CC1(C)c2ccccc2-c2ccc(N(c3ccc(-c4cccc5ccccc45)cc3)c3ccc4cccc5c4c3-c3cc(-c4ccccc4)ccc3C5(C)C)cc21 ; CC1(C)c2ccc(-c3ccccc3)cc2-c2c(N(c3ccc(-c4ccccc4)cc3)c3ccc4c(c3)C(c3ccccc3)(c3ccccc3)c3ccccc3-4)ccc3cccc1c23]

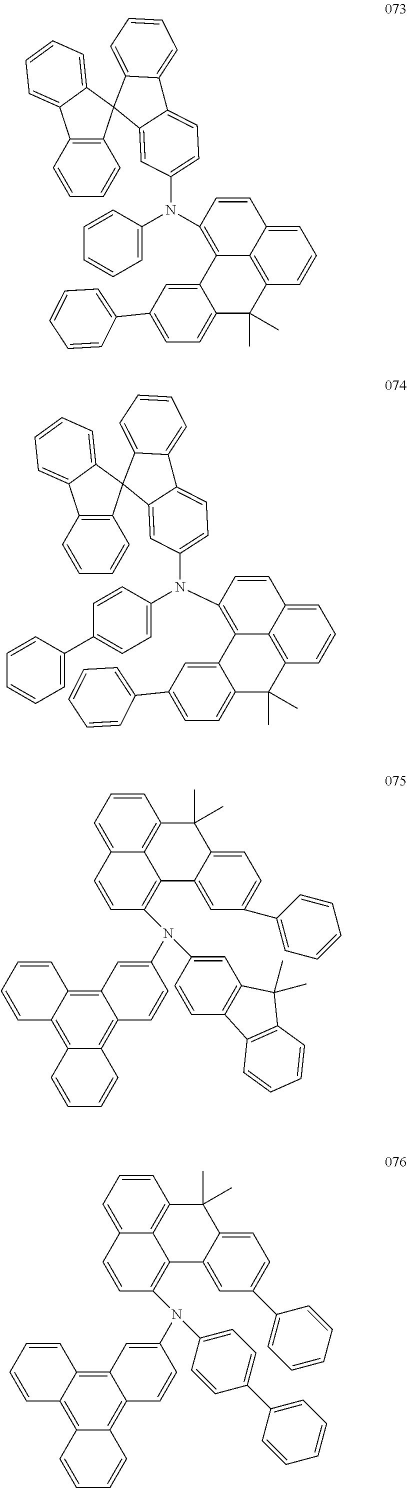 [SMILES: CC1(C)c2ccc(-c3ccccc3)cc2-c2c(N(c3ccccc3)c3ccc4c(c3)C3(c5ccccc5-c5ccccc53)c3ccccc3-4)ccc3cccc1c23 ; CC1(C)c2ccc(-c3ccccc3)cc2-c2c(N(c3ccc(-c4ccccc4)cc3)c3ccc4c(c3)C3(c5ccccc5-c5ccccc53)c3ccccc3-4)ccc3cccc1c23 ; CC1(C)c2ccccc2-c2ccc(N(c3ccc4c5ccccc5c5ccccc5c4c3)c3ccc4cccc5c4c3-c3cc(-c4ccccc4)ccc3C5(C)C)cc21 ; CC1(C)c2ccc(-c3ccccc3)cc2-c2c(N(c3ccc(-c4ccccc4)cc3)c3ccc4c5ccccc5c5ccccc5c4c3)ccc3cccc1c23]

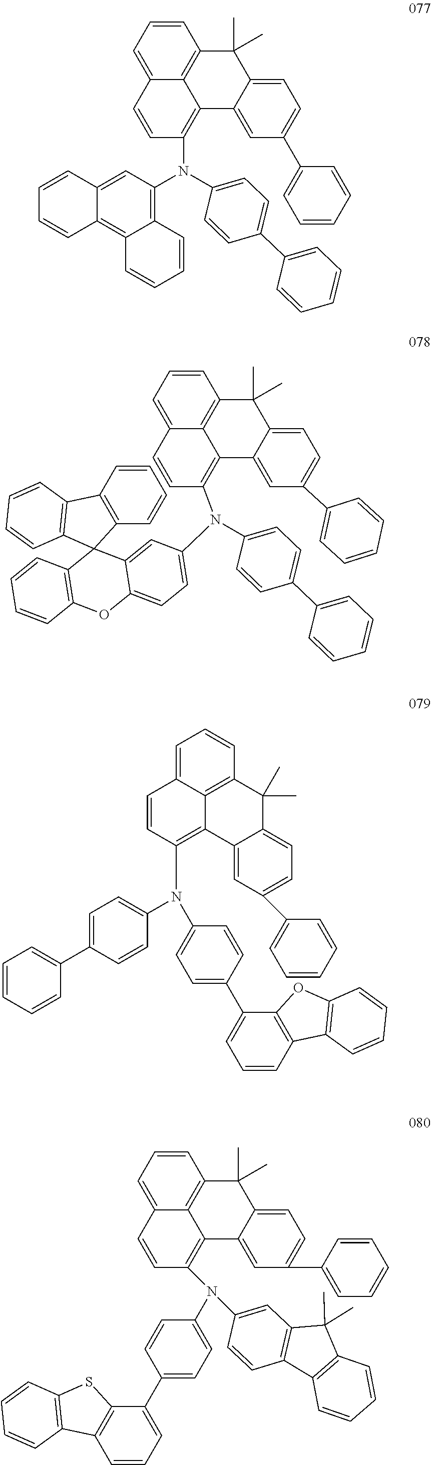 [SMILES: CC1(C)c2ccc(-c3ccccc3)cc2-c2c(N(c3ccc(-c4ccccc4)cc3)c3cc4ccccc4c4ccccc34)ccc3cccc1c23 ; CC1(C)c2ccc(-c3ccccc3)cc2-c2c(N(c3ccc(-c4ccccc4)cc3)c3ccc4c(c3)C3(c5ccccc5O4)c4ccccc4-c4ccccc43)ccc3cccc1c23 ; CC1(C)c2ccc(-c3ccccc3)cc2-c2c(N(c3ccc(-c4ccccc4)cc3)c3ccc(-c4cccc5c4oc4ccccc45)cc3)ccc3cccc1c23 ; CC1(C)c2ccccc2-c2ccc(N(c3ccc(-c4cccc5c4sc4ccccc45)cc3)c3ccc4cccc5c4c3-c3cc(-c4ccccc4)ccc3C5(C)C)cc21]

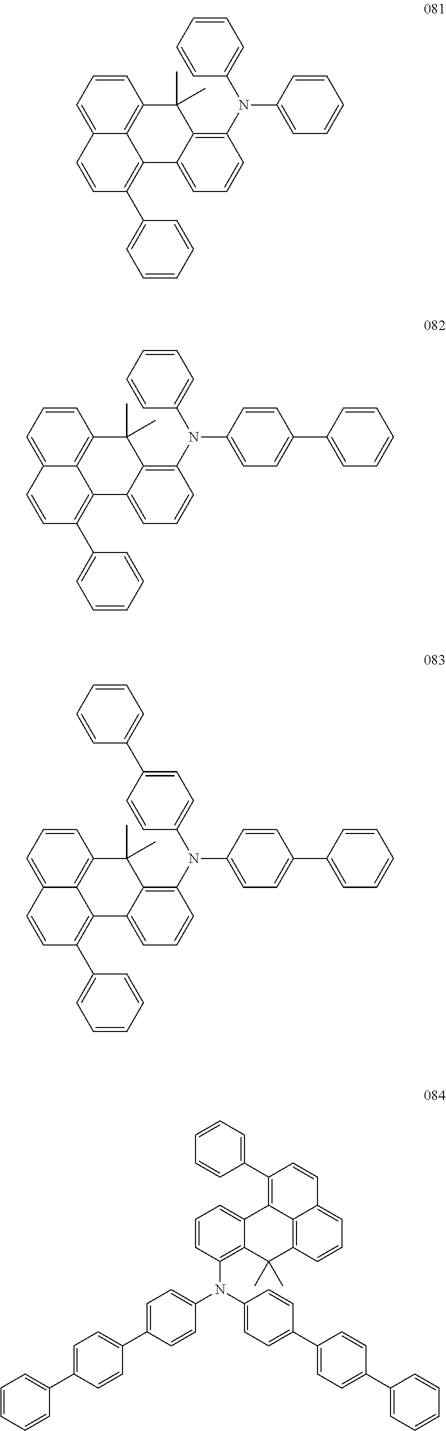 [SMILES: CC1(C)c2c(cccc2N(c2ccccc2)c2ccccc2)-c2c(-c3ccccc3)ccc3cccc1c23 ; CC1(C)c2c(cccc2N(c2ccccc2)c2ccc(-c3ccccc3)cc2)-c2c(-c3ccccc3)ccc3cccc1c23 ; CC1(C)c2c(cccc2N(c2ccc(-c3ccccc3)cc2)c2ccc(-c3ccccc3)cc2)-c2c(-c3ccccc3)ccc3cccc1c23 ; CC1(C)c2c(cccc2N(c2ccc(-c3ccc(-c4ccccc4)cc3)cc2)c2ccc(-c3ccc(-c4ccccc4)cc3)cc2)-c2c(-c3ccccc3)ccc3cccc1c23]

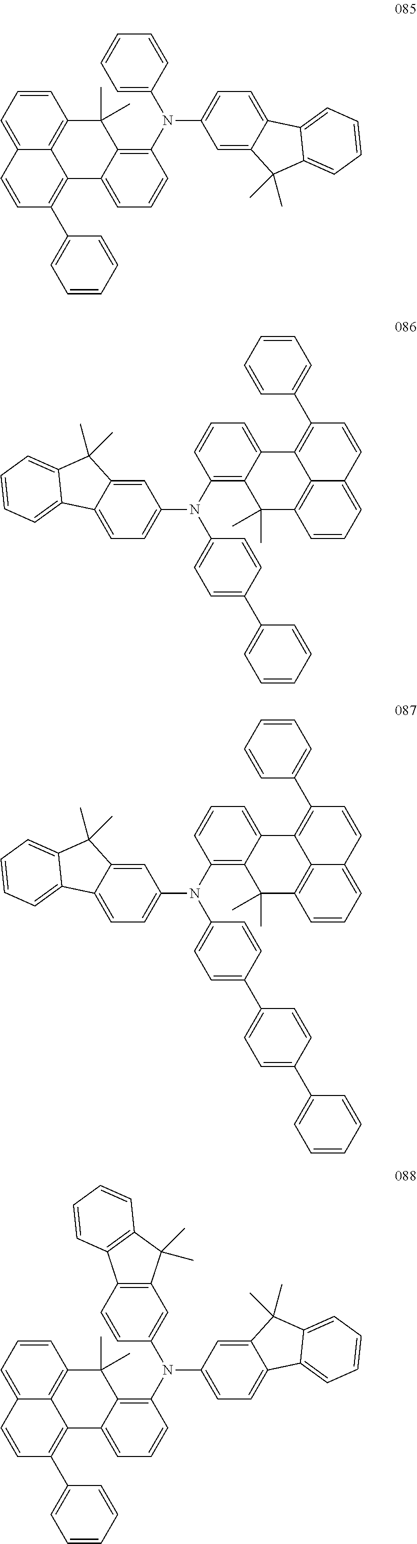

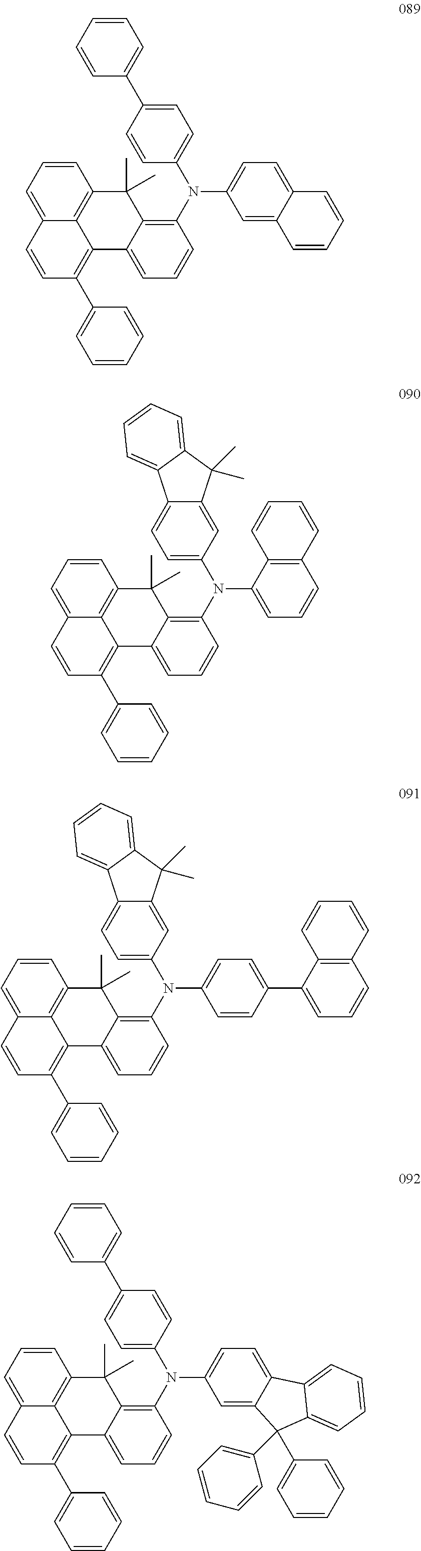

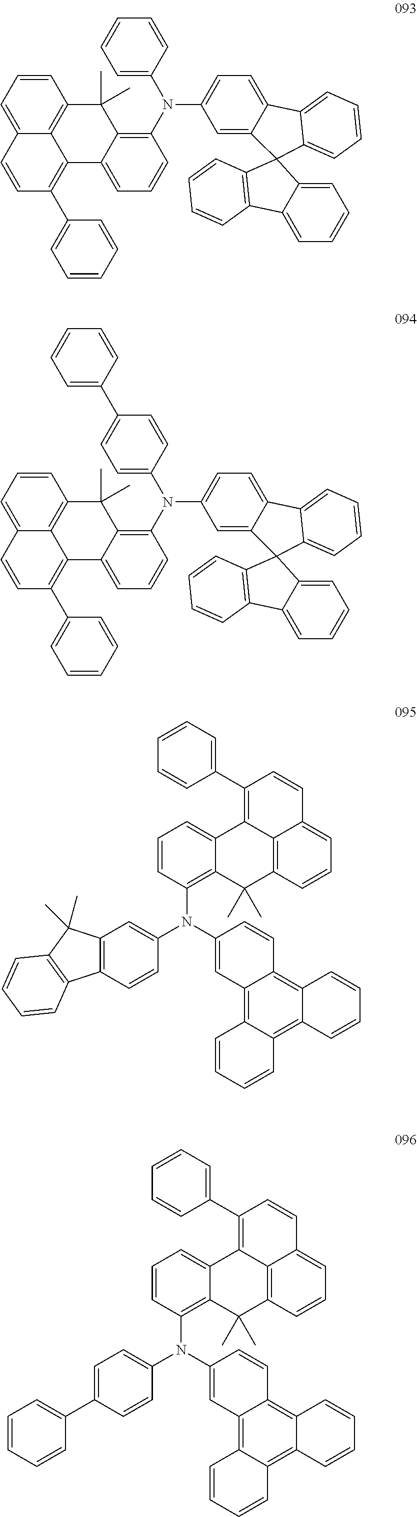 [SMILES: CC1(C)c2c(cccc2N(c2ccccc2)c2ccc3c(c2)C2(c4ccccc4-c4ccccc42)c2ccccc2-3)-c2c(-c3ccccc3)ccc3cccc1c23 ; CC1(C)c2c(cccc2N(c2ccc(-c3ccccc3)cc2)c2ccc3c(c2)C2(c4ccccc4-c4ccccc42)c2ccccc2-3)-c2c(-c3ccccc3)ccc3cccc1c23 ; CC1(C)c2ccccc2-c2ccc(N(c3ccc4c5ccccc5c5ccccc5c4c3)c3cccc4c3C(C)(C)c3cccc5ccc(-c6ccccc6)c-4c35)cc21 ; CC1(C)c2c(cccc2N(c2ccc(-c3ccccc3)cc2)c2ccc3c4ccccc4c4ccccc4c3c2)-c2c(-c3ccccc3)ccc3cccc1c23]

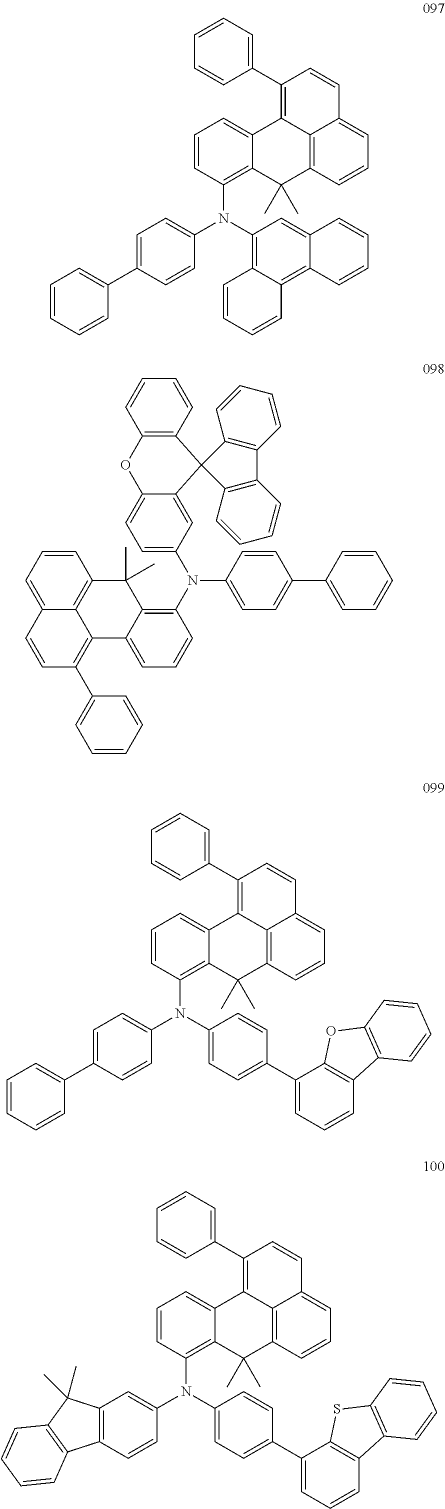 [SMILES: CC1(C)c2c(cccc2N(c2ccc(-c3ccccc3)cc2)c2cc3ccccc3c3ccccc23)-c2c(-c3ccccc3)ccc3cccc1c23 ; CC1(C)c2c(cccc2N(c2ccc(-c3ccccc3)cc2)c2ccc3c(c2)C2(c4ccccc4O3)c3ccccc3-c3ccccc32)-c2c(-c3ccccc3)ccc3cccc1c23 ; CC1(C)c2c(cccc2N(c2ccc(-c3ccccc3)cc2)c2ccc(-c3cccc4c3oc3ccccc34)cc2)-c2c(-c3ccccc3)ccc3cccc1c23 ; CC1(C)c2ccccc2-c2ccc(N(c3ccc(-c4cccc5c4sc4ccccc45)cc3)c3cccc4c3C(C)(C)c3cccc5ccc(-c6ccccc6)c-4c35)cc21]

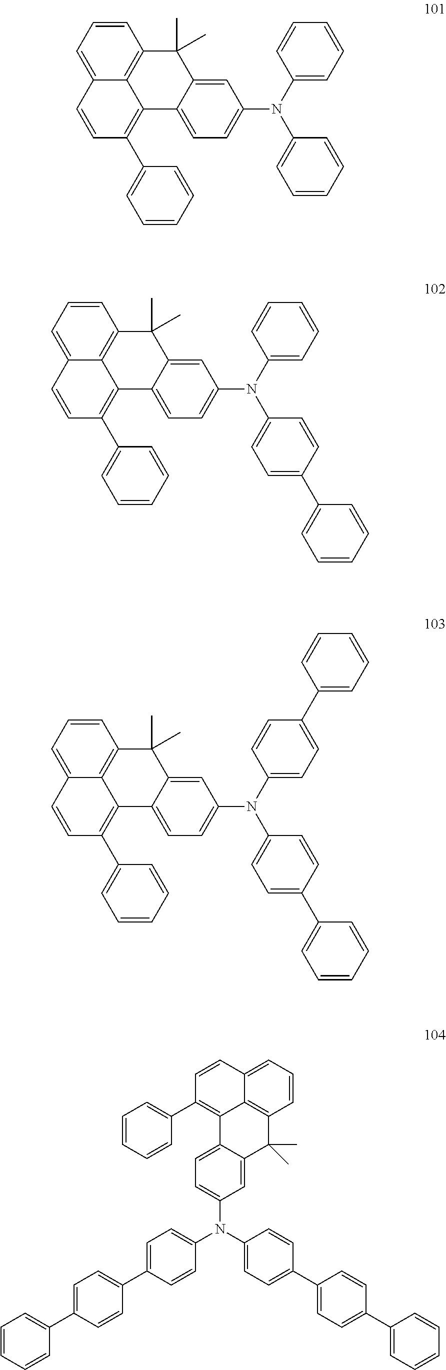 [SMILES: CC1(C)c2cc(N(c3ccccc3)c3ccccc3)ccc2-c2c(-c3ccccc3)ccc3cccc1c23 ; CC1(C)c2cc(N(c3ccccc3)c3ccc(-c4ccccc4)cc3)ccc2-c2c(-c3ccccc3)ccc3cccc1c23 ; CC1(C)c2cc(N(c3ccc(-c4ccccc4)cc3)c3ccc(-c4ccccc4)cc3)ccc2-c2c(-c3ccccc3)ccc3cccc1c23 ; CC1(C)c2cc(N(c3ccc(-c4ccc(-c5ccccc5)cc4)cc3)c3ccc(-c4ccc(-c5ccccc5)cc4)cc3)ccc2-c2c(-c3ccccc3)ccc3cccc1c23]

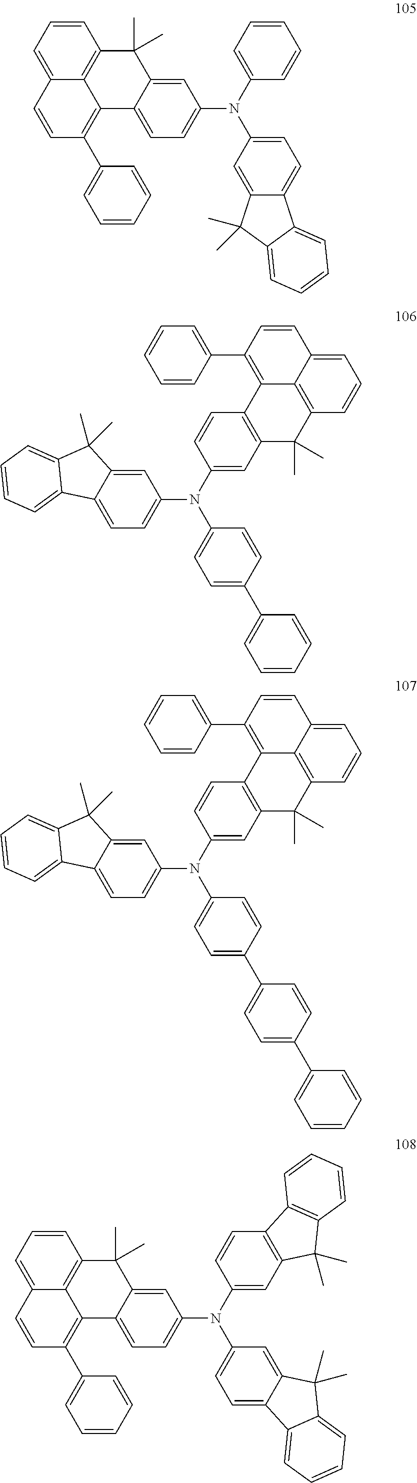

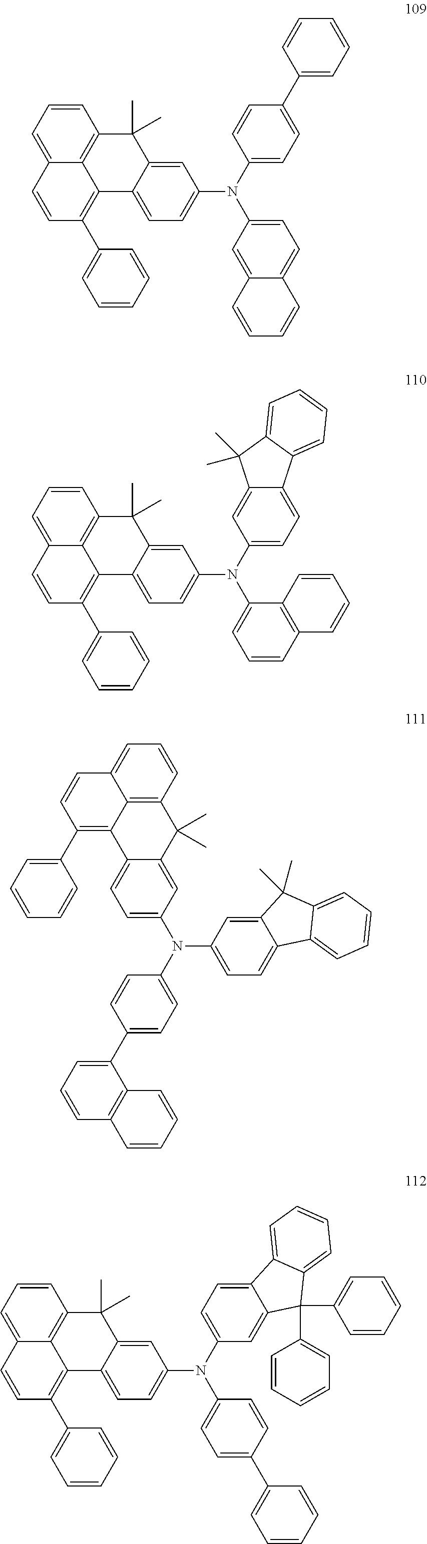 [SMILES: CC1(C)c2cc(N(c3ccc(-c4ccccc4)cc3)c3ccc4ccccc4c3)ccc2-c2c(-c3ccccc3)ccc3cccc1c23 ; CC1(C)c2ccccc2-c2ccc(N(c3ccc4c(c3)C(C)(C)c3cccc5ccc(-c6ccccc6)c-4c35)c3cccc4ccccc34)cc21 ; CC1(C)c2ccccc2-c2ccc(N(c3ccc(-c4cccc5ccccc45)cc3)c3ccc4c(c3)C(C)(C)c3cccc5ccc(-c6ccccc6)c-4c35)cc21 ; CC1(C)c2cc(N(c3ccc(-c4ccccc4)cc3)c3ccc4c(c3)C(c3ccccc3)(c3ccccc3)c3ccccc3-4)ccc2-c2c(-c3ccccc3)ccc3cccc1c23]

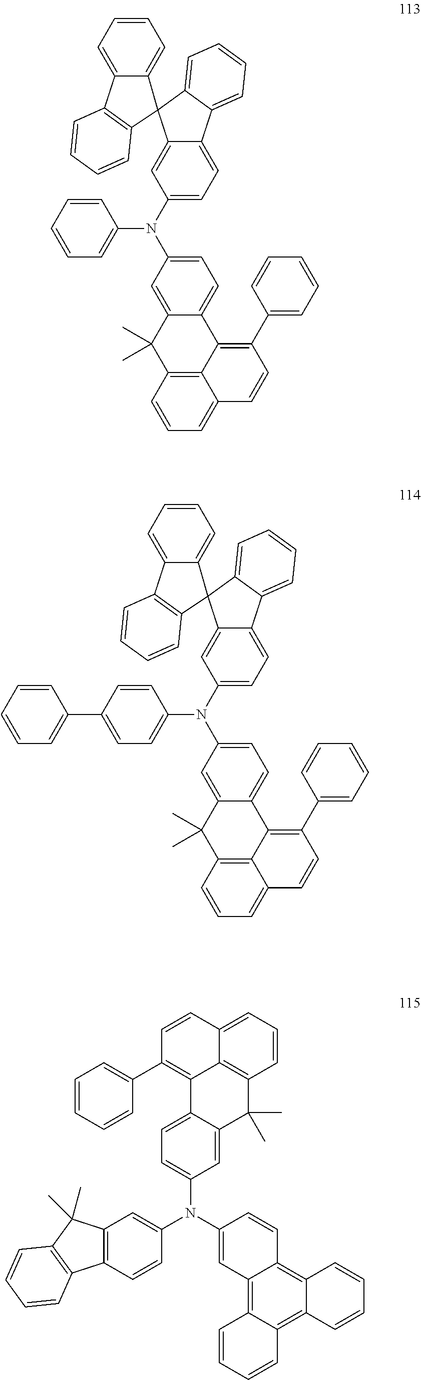 [SMILES: CC1(C)c2cc(N(c3ccccc3)c3ccc4c(c3)C3(c5ccccc5-c5ccccc53)c3ccccc3-4)ccc2-c2c(-c3ccccc3)ccc3cccc1c23 ; CC1(C)c2cc(N(c3ccc(-c4ccccc4)cc3)c3ccc4c(c3)C3(c5ccccc5-c5ccccc53)c3ccccc3-4)ccc2-c2c(-c3ccccc3)ccc3cccc1c23 ; CC1(C)c2ccccc2-c2ccc(N(c3ccc4c(c3)C(C)(C)c3cccc5ccc(-c6ccccc6)c-4c35)c3ccc4c5ccccc5c5ccccc5c4c3)cc21]

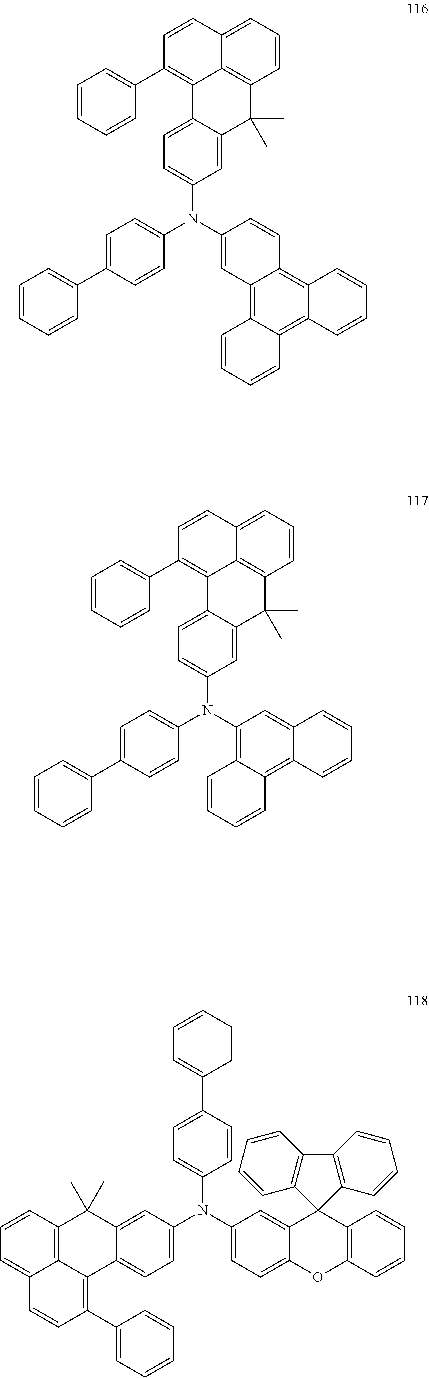 [SMILES: CC1(C)c2cc(N(c3ccc(-c4ccccc4)cc3)c3ccc4c5ccccc5c5ccccc5c4c3)ccc2-c2c(-c3ccccc3)ccc3cccc1c23 ; CC1(C)c2cc(N(c3ccc(-c4ccccc4)cc3)c3cc4ccccc4c4ccccc34)ccc2-c2c(-c3ccccc3)ccc3cccc1c23 ; CC1(C)c2cc(N(c3ccc(C4=CC=CCC4)cc3)c3ccc4c(c3)C3(c5ccccc5O4)c4ccccc4-c4ccccc43)ccc2-c2c(-c3ccccc3)ccc3cccc1c23]

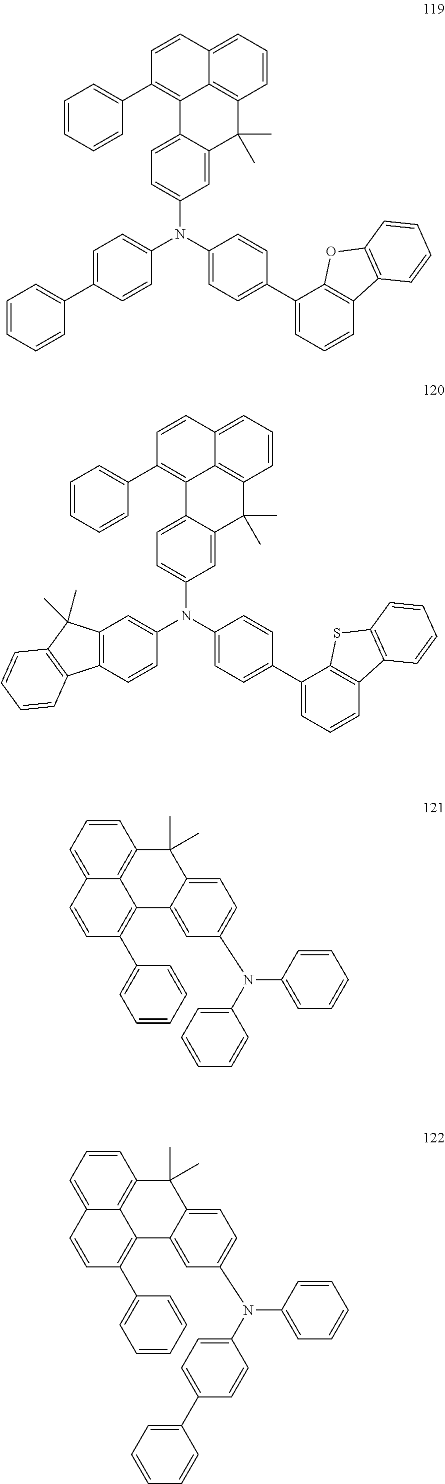 [SMILES: CC1(C)c2cc(N(c3ccc(-c4ccccc4)cc3)c3ccc(-c4cccc5c4oc4ccccc45)cc3)ccc2-c2c(-c3ccccc3)ccc3cccc1c23 ; CC1(C)c2ccccc2-c2ccc(N(c3ccc(-c4cccc5c4sc4ccccc45)cc3)c3ccc4c(c3)C(C)(C)c3cccc5ccc(-c6ccccc6)c-4c35)cc21 ; CC1(C)c2ccc(N(c3ccccc3)c3ccccc3)cc2-c2c(-c3ccccc3)ccc3cccc1c23 ; CC1(C)c2ccc(N(c3ccccc3)c3ccc(-c4ccccc4)cc3)cc2-c2c(-c3ccccc3)ccc3cccc1c23]

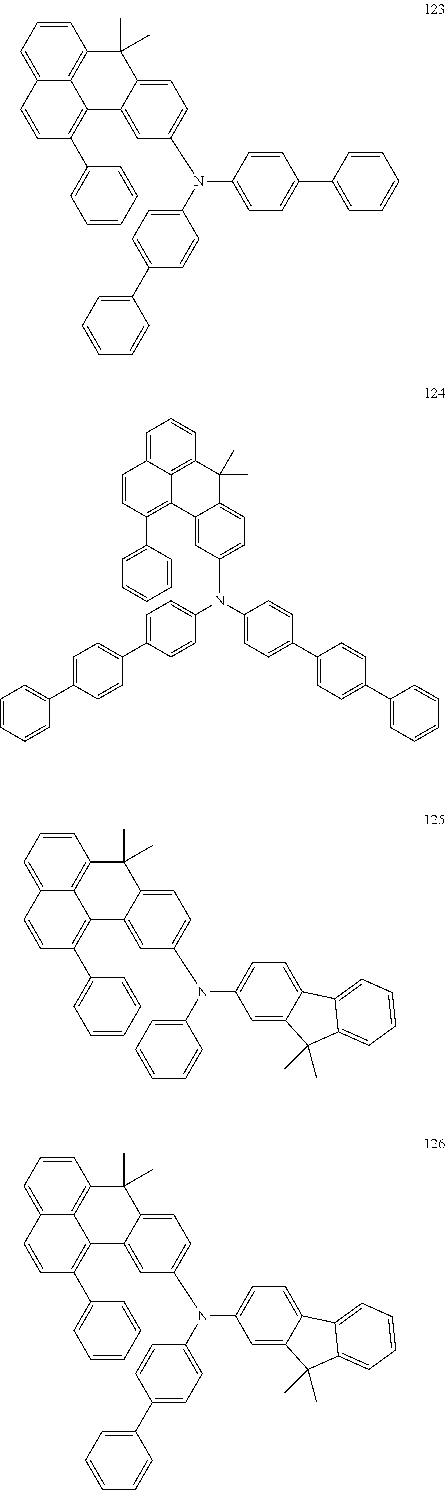 [SMILES: CC1(C)c2ccc(N(c3ccc(-c4ccccc4)cc3)c3ccc(-c4ccccc4)cc3)cc2-c2c(-c3ccccc3)ccc3cccc1c23 ; CC1(C)c2ccc(N(c3ccc(-c4ccc(-c5ccccc5)cc4)cc3)c3ccc(-c4ccc(-c5ccccc5)cc4)cc3)cc2-c2c(-c3ccccc3)ccc3cccc1c23 ; CC1(C)c2ccccc2-c2ccc(N(c3ccccc3)c3ccc4c(c3)-c3c(-c5ccccc5)ccc5cccc(c35)C4(C)C)cc21 ; CC1(C)c2ccccc2-c2ccc(N(c3ccc(-c4ccccc4)cc3)c3ccc4c(c3)-c3c(-c5ccccc5)ccc5cccc(c35)C4(C)C)cc21]

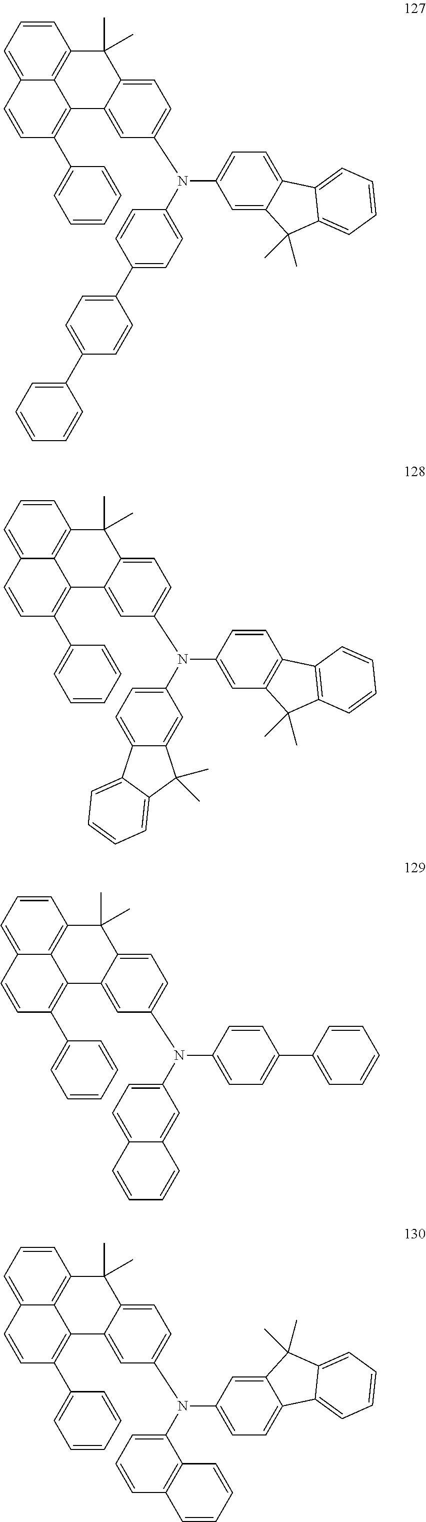 [SMILES: CC1(C)c2ccccc2-c2ccc(N(c3ccc(-c4ccc(-c5ccccc5)cc4)cc3)c3ccc4c(c3)-c3c(-c5ccccc5)ccc5cccc(c35)C4(C)C)cc21 ; CC1(C)c2ccccc2-c2ccc(N(c3ccc4c(c3)-c3c(-c5ccccc5)ccc5cccc(c35)C4(C)C)c3ccc4c(c3)C(C)(C)c3ccccc3-4)cc21 ; CC1(C)c2ccc(N(c3ccc(-c4ccccc4)cc3)c3ccc4ccccc4c3)cc2-c2c(-c3ccccc3)ccc3cccc1c23 ; CC1(C)c2ccccc2-c2ccc(N(c3ccc4c(c3)-c3c(-c5ccccc5)ccc5cccc(c35)C4(C)C)c3cccc4ccccc34)cc21]

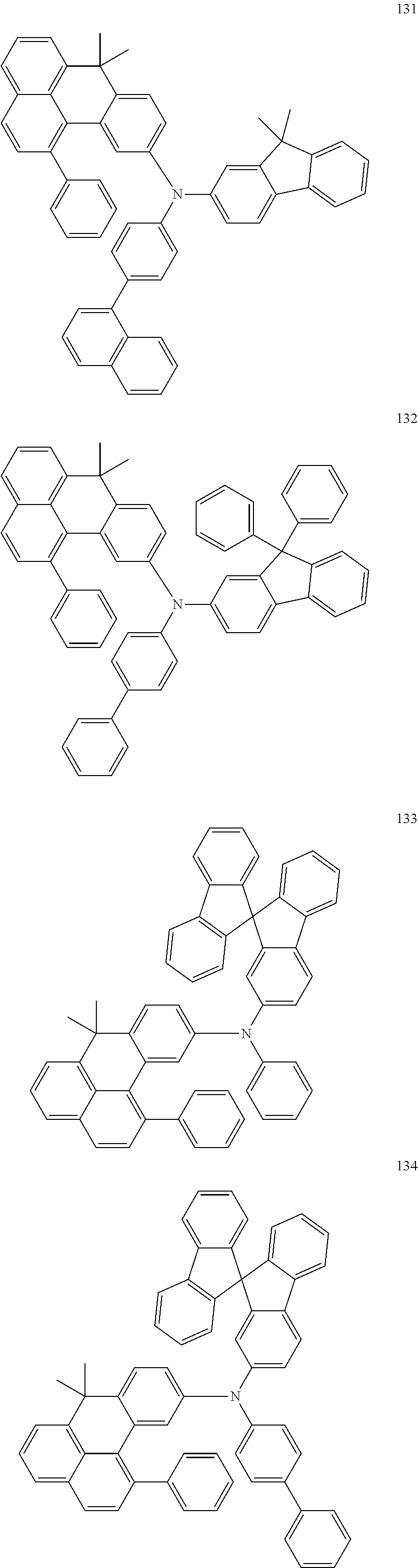

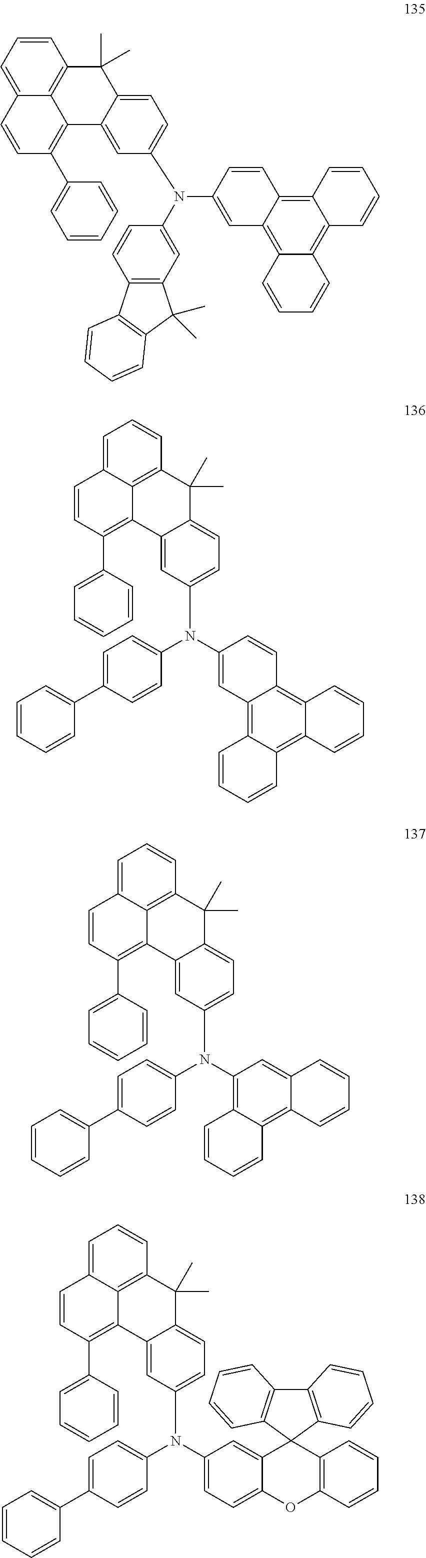

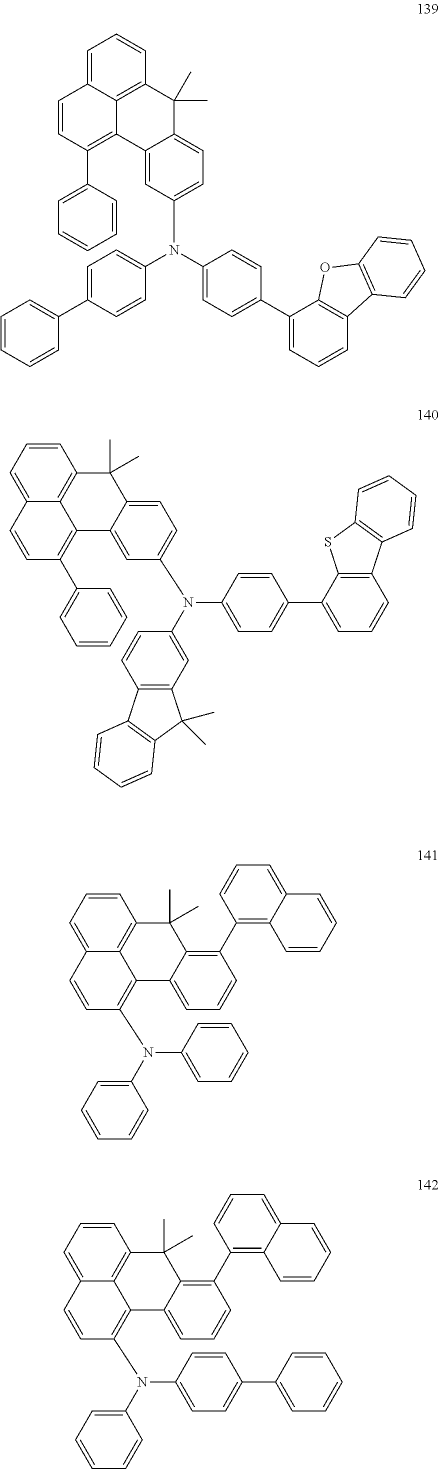 [SMILES: CC1(C)c2ccc(N(c3ccc(-c4ccccc4)cc3)c3ccc(-c4cccc5c4oc4ccccc45)cc3)cc2-c2c(-c3ccccc3)ccc3cccc1c23 ; CC1(C)c2ccccc2-c2ccc(N(c3ccc(-c4cccc5c4sc4ccccc45)cc3)c3ccc4c(c3)-c3c(-c5ccccc5)ccc5cccc(c35)C4(C)C)cc21 ; CC1(C)c2c(-c3cccc4ccccc34)cccc2-c2c(N(c3ccccc3)c3ccccc3)ccc3cccc1c23 ; CC1(C)c2c(-c3cccc4ccccc34)cccc2-c2c(N(c3ccccc3)c3ccc(-c4ccccc4)cc3)ccc3cccc1c23]

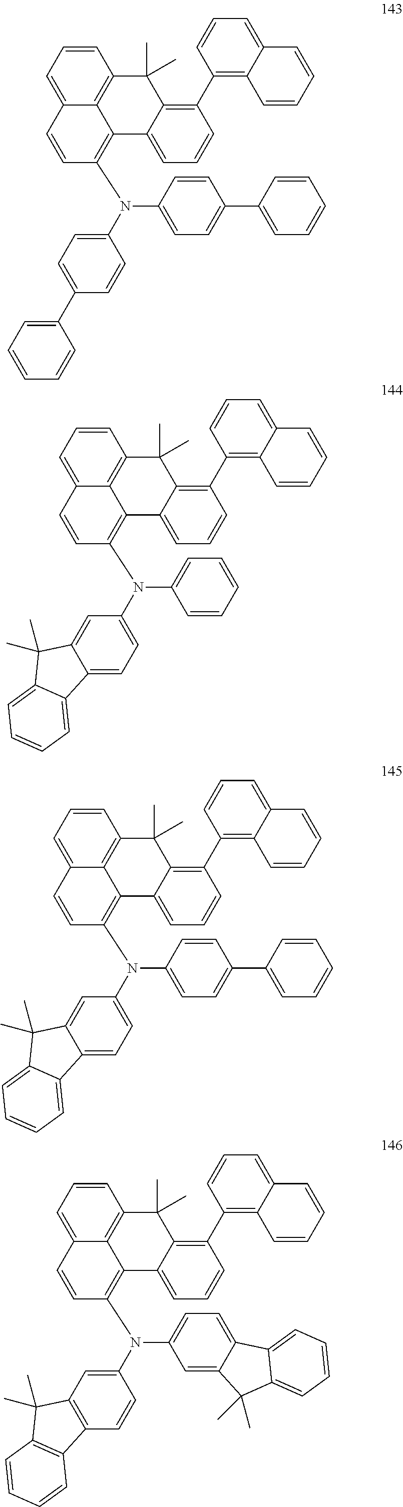 [SMILES: CC1(C)c2c(-c3cccc4ccccc34)cccc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3cccc(-c4cccc6ccccc46)c3C5(C)C)cc21 ; CC1(C)c2ccccc2-c2ccc(N(c3ccc(-c4ccccc4)cc3)c3ccc4cccc5c4c3-c3cccc(-c4cccc6ccccc46)c3C5(C)C)cc21 ; CC1(C)c2ccccc2-c2ccc(N(c3ccc4c(c3)C(C)(C)c3ccccc3-4)c3ccc4cccc5c4c3-c3cccc(-c4cccc6ccccc46)c3C5(C)C)cc21]

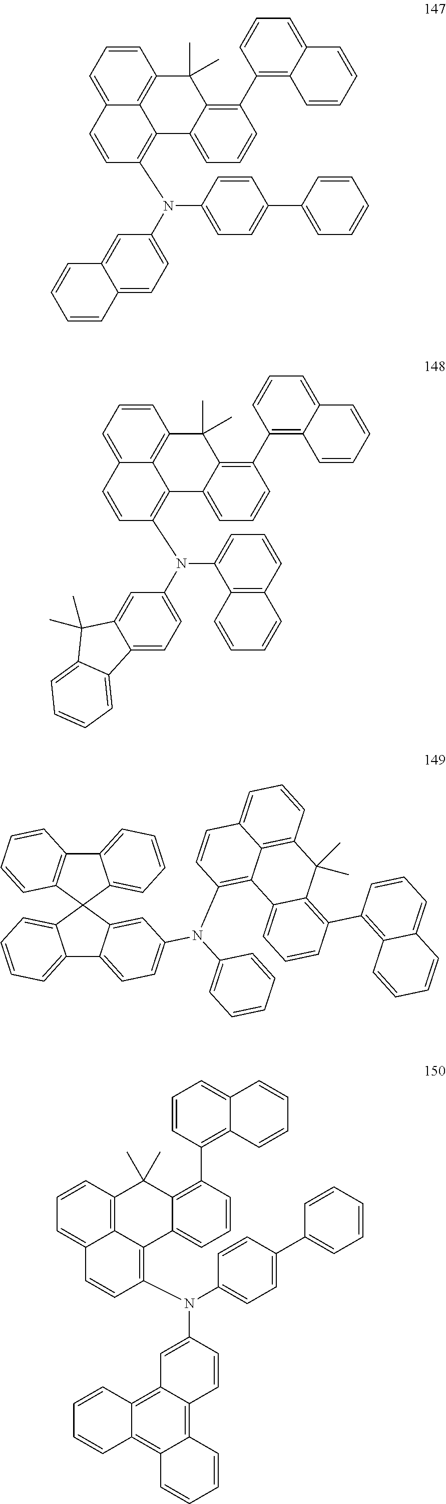 [SMILES: CC1(C)c2c(-c3cccc4ccccc34)cccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cccc(-c4cccc6ccccc46)c3C5(C)C)c3cccc4ccccc34)cc21 ; CC1(C)c2c(-c3cccc4ccccc34)cccc2-c2c(N(c3ccccc3)c3ccc4c(c3)C3(c5ccccc5-c5ccccc53)c3ccccc3-4)ccc3cccc1c23 ; CC1(C)c2c(-c3cccc4ccccc34)cccc2-c2c(N(c3ccc(-c4ccccc4)cc3)c3ccc4c5ccccc5c5ccccc5c4c3)ccc3cccc1c23]

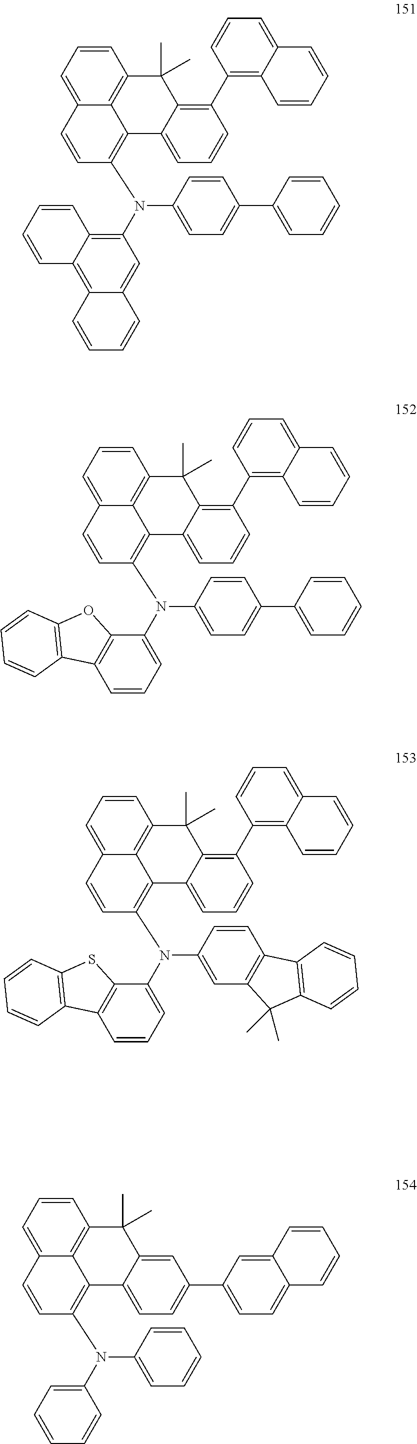 [SMILES: CC1(C)c2c(-c3cccc4ccccc34)cccc2-c2c(N(c3ccc(-c4ccccc4)cc3)c3cc4ccccc4c4ccccc34)ccc3cccc1c23 ; CC1(C)c2c(-c3cccc4ccccc34)cccc2-c2c(N(c3ccc(-c4ccccc4)cc3)c3cccc4c3oc3ccccc34)ccc3cccc1c23 ; CC1(C)c2ccccc2-c2ccc(N(c3ccc4cccc5c4c3-c3cccc(-c4cccc6ccccc46)c3C5(C)C)c3cccc4c3sc3ccccc34)cc21 ; CC1(C)c2cc(-c3ccc4ccccc4c3)ccc2-c2c(N(c3ccccc3)c3ccccc3)ccc3cccc1c23]

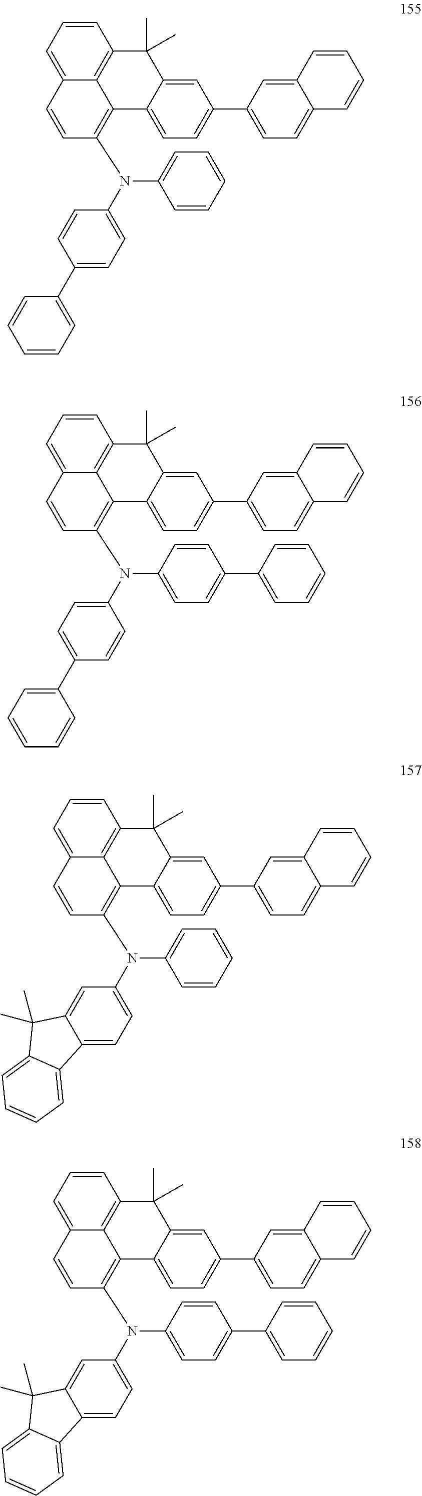 [SMILES: CC1(C)c2cc(-c3ccc4ccccc4c3)ccc2-c2c(N(c3ccccc3)c3ccc(-c4ccccc4)cc3)ccc3cccc1c23 ; CC1(C)c2cc(-c3ccc4ccccc4c3)ccc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3ccc(-c4ccc6ccccc6c4)cc3C5(C)C)cc21 ; CC1(C)c2ccccc2-c2ccc(N(c3ccc(-c4ccccc4)cc3)c3ccc4cccc5c4c3-c3ccc(-c4ccc6ccccc6c4)cc3C5(C)C)cc21]

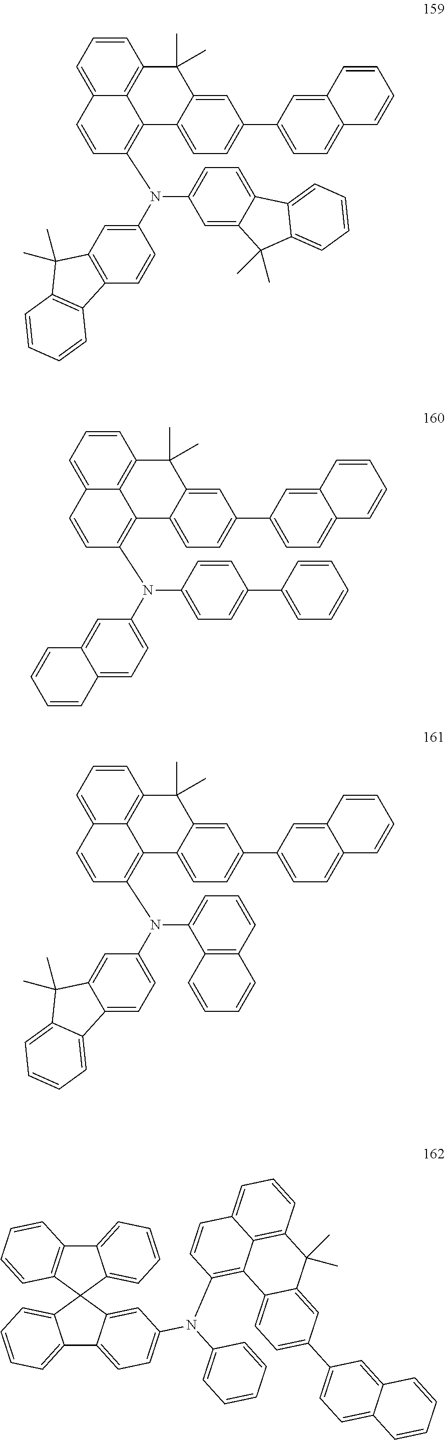 [SMILES: CC1(C)c2ccccc2-c2ccc(N(c3ccc4c(c3)C(C)(C)c3ccccc3-4)c3ccc4cccc5c4c3-c3ccc(-c4ccc6ccccc6c4)cc3C5(C)C)cc21 ; CC1(C)c2cc(-c3ccc4ccccc4c3)ccc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3ccc(-c4ccc6ccccc6c4)cc3C5(C)C)c3cccc4ccccc34)cc21 ; CC1(C)c2cc(-c3ccc4ccccc4c3)ccc2-c2c(N(c3ccccc3)c3ccc4c(c3)C3(c5ccccc5-c5ccccc53)c3ccccc3-4)ccc3cccc1c23]

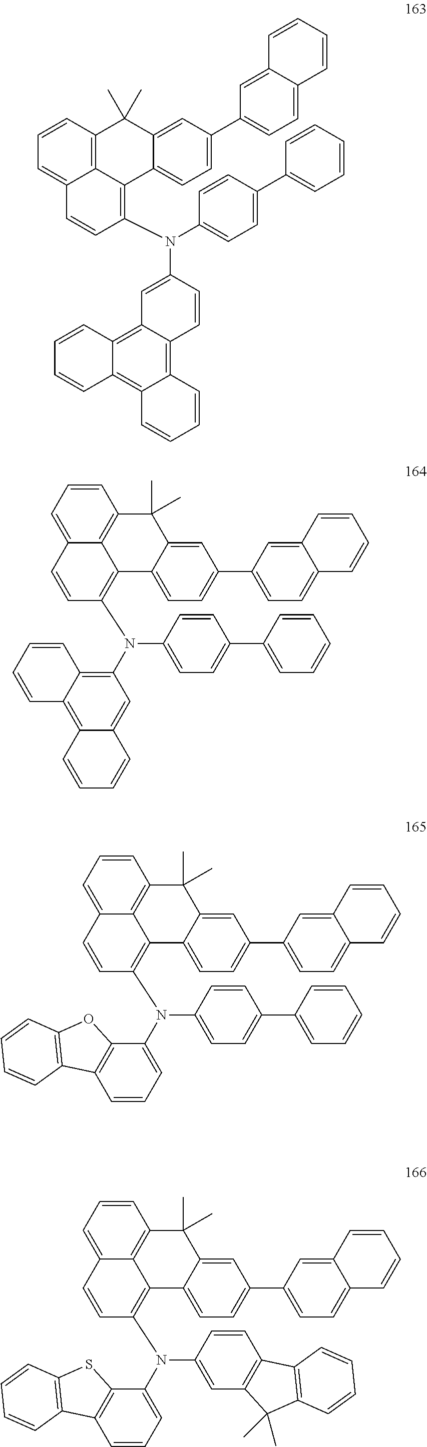

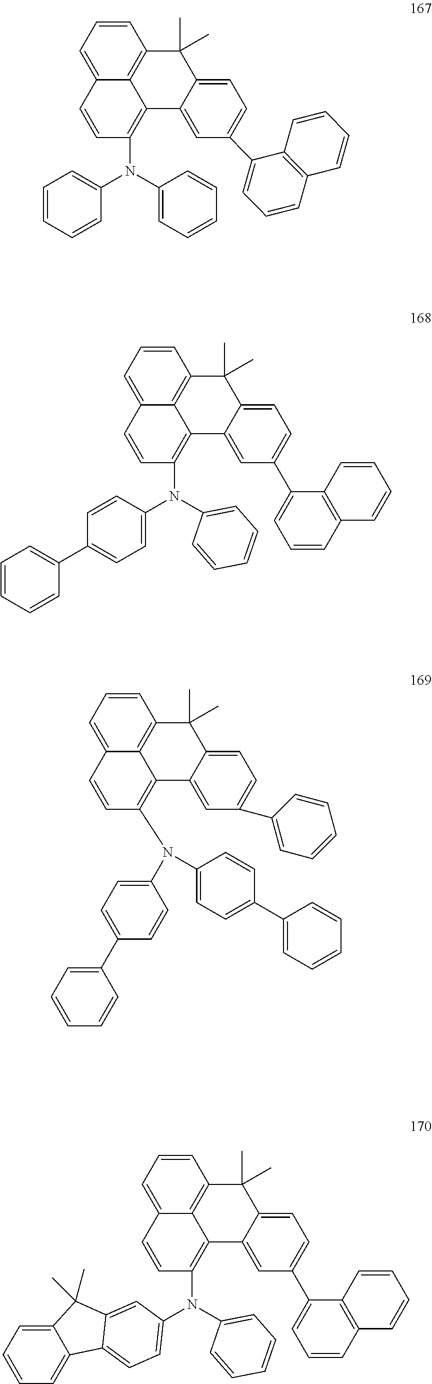 [SMILES: CC1(C)c2ccc(-c3cccc4ccccc34)cc2-c2c(N(c3ccccc3)c3ccccc3)ccc3cccc1c23 ; CC1(C)c2ccc(-c3cccc4ccccc34)cc2-c2c(N(c3ccccc3)c3ccc(-c4ccccc4)cc3)ccc3cccc1c23 ; CC1(C)c2ccc(-c3ccccc3)cc2-c2c(N(c3ccc(-c4ccccc4)cc3)c3ccc(-c4ccccc4)cc3)ccc3cccc1c23 ; CC1(C)c2ccccc2-c2ccc(N(c3ccccc3)c3ccc4cccc5c4c3-c3cc(-c4cccc6ccccc46)ccc3C5(C)C)cc21]

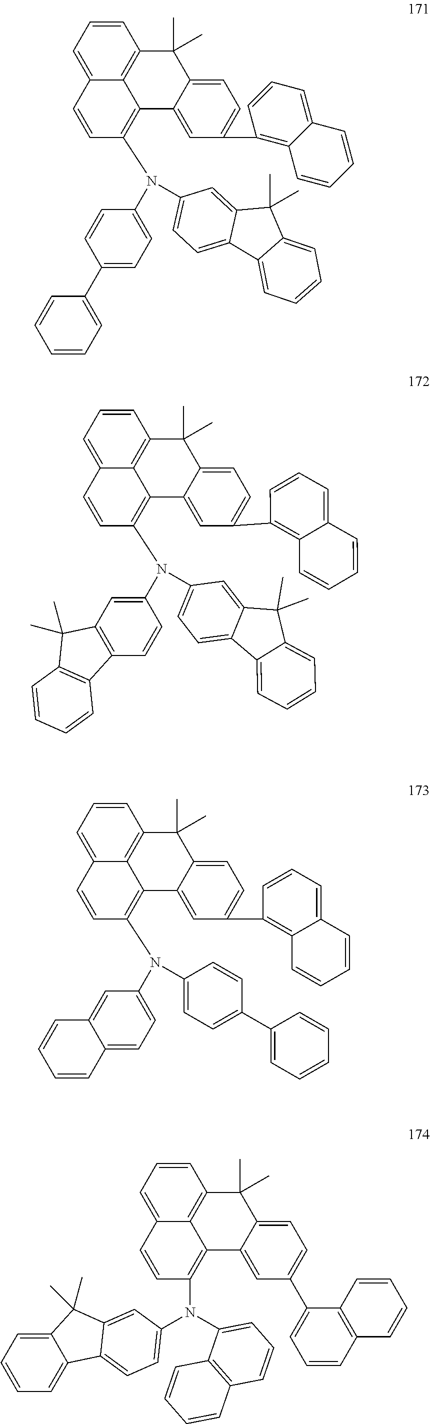 [SMILES: CC1(C)c2ccccc2-c2ccc(N(c3ccc(-c4ccccc4)cc3)c3ccc4cccc5c4c3-c3cc(-c4cccc6ccccc46)ccc3C5(C)C)cc21 ; CC1(C)c2ccccc2-c2ccc(N(c3ccc4c(c3)C(C)(C)c3ccccc3-4)c3ccc4cccc5c4c3-c3cc(-c4cccc6ccccc46)ccc3C5(C)C)cc21 ; CC1(C)c2ccc(-c3cccc4ccccc34)cc2-c2c(N(c3ccc(-c4ccccc4)cc3)c3ccc4ccccc4c3)ccc3cccc1c23 ; CC1(C)c2ccccc2-c2ccc(N(c3ccc4cccc5c4c3-c3cc(-c4cccc6ccccc46)ccc3C5(C)C)c3cccc4ccccc34)cc21]

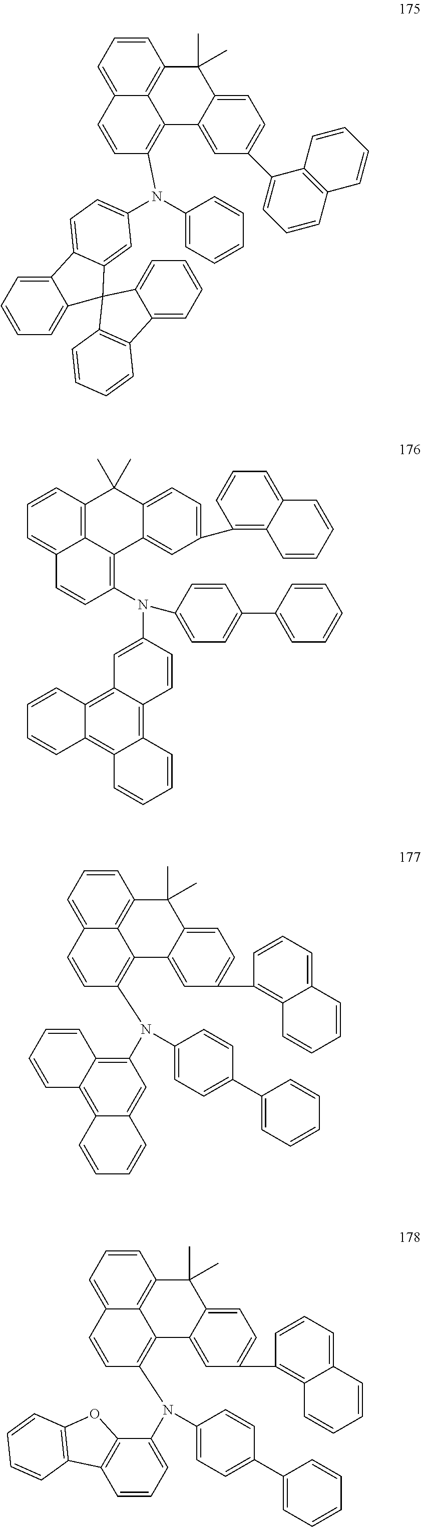 [SMILES: CC1(C)c2ccc(-c3cccc4ccccc34)cc2-c2c(N(c3ccccc3)c3ccc4c(c3)C3(c5ccccc5-c5ccccc53)c3ccccc3-4)ccc3cccc1c23 ; CC1(C)c2ccc(-c3cccc4ccccc34)cc2-c2c(N(c3ccc(-c4ccccc4)cc3)c3ccc4c5ccccc5c5ccccc5c4c3)ccc3cccc1c23 ; CC1(C)c2ccc(-c3cccc4ccccc34)cc2-c2c(N(c3ccc(-c4ccccc4)cc3)c3cc4ccccc4c4ccccc34)ccc3cccc1c23 ; CC1(C)c2ccc(-c3cccc4ccccc34)cc2-c2c(N(c3ccc(-c4ccccc4)cc3)c3cccc4c3oc3ccccc34)ccc3cccc1c23]

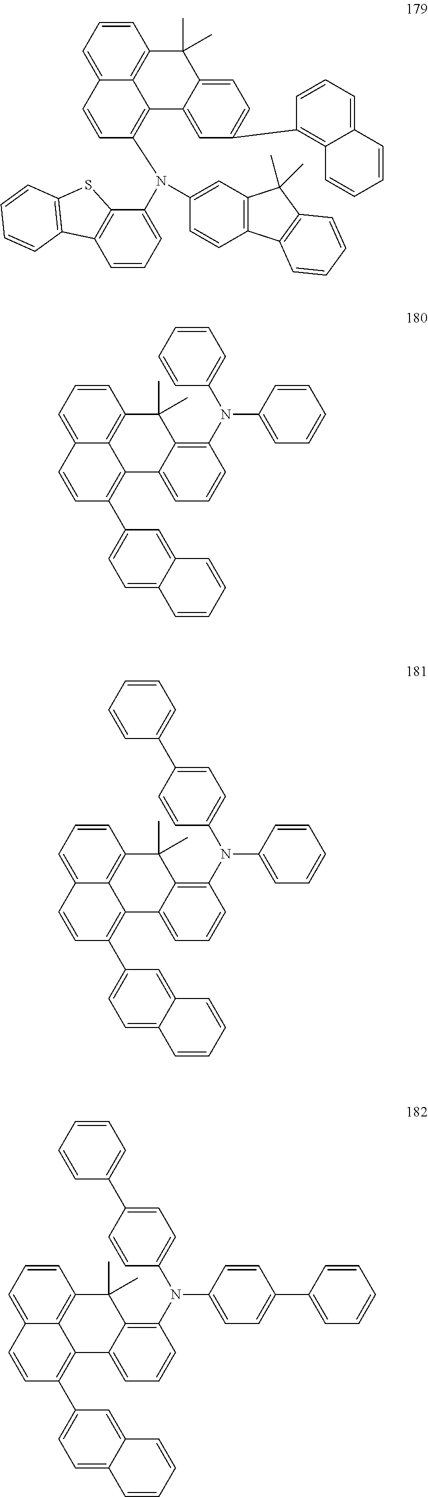 [SMILES: CC1(C)c2ccccc2-c2ccc(N(c3ccc4cccc5c4c3-c3cc(-c4cccc6ccccc46)ccc3C5(C)C)c3cccc4c3sc3ccccc34)cc21 ; CC1(C)c2c(cccc2N(c2ccccc2)c2ccccc2)-c2c(-c3ccc4ccccc4c3)ccc3cccc1c23 ; CC1(C)c2c(cccc2N(c2ccccc2)c2ccc(-c3ccccc3)cc2)-c2c(-c3ccc4ccccc4c3)ccc3cccc1c23 ; CC1(C)c2c(cccc2N(c2ccc(-c3ccccc3)cc2)c2ccc(-c3ccccc3)cc2)-c2c(-c3ccc4ccccc4c3)ccc3cccc1c23]

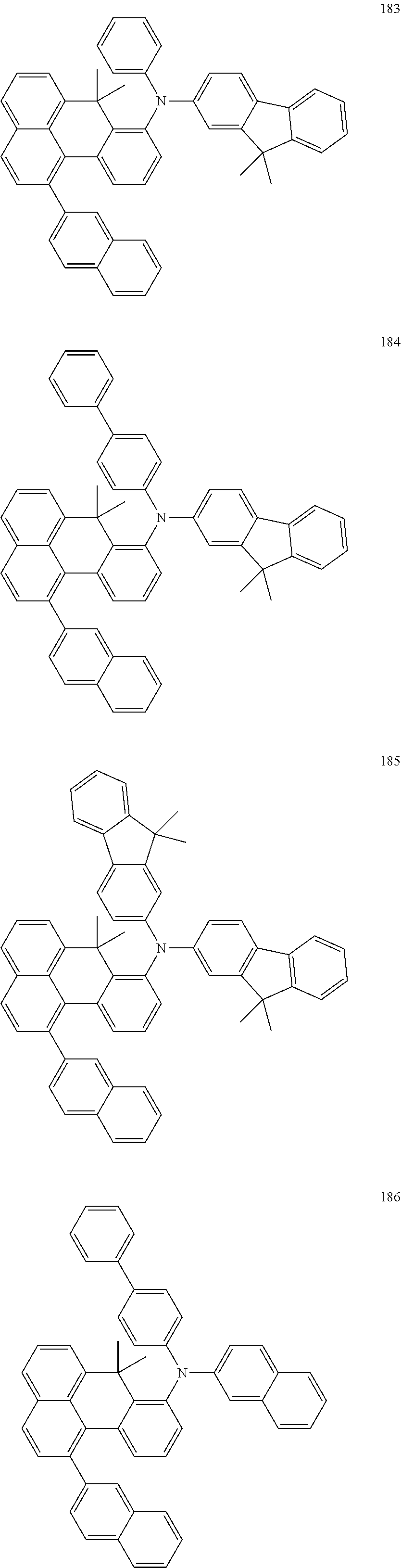 [SMILES: CC1(C)c2ccccc2-c2ccc(N(c3ccccc3)c3cccc4c3C(C)(C)c3cccc5ccc(-c6ccc7ccccc7c6)c-4c35)cc21 ; CC1(C)c2ccccc2-c2ccc(N(c3ccc(-c4ccccc4)cc3)c3cccc4c3C(C)(C)c3cccc5ccc(-c6ccc7ccccc7c6)c-4c35)cc21 ; CC1(C)c2ccccc2-c2ccc(N(c3ccc4c(c3)C(C)(C)c3ccccc3-4)c3cccc4c3C(C)(C)c3cccc5ccc(-c6ccc7ccccc7c6)c-4c35)cc21 ; CC1(C)c2c(cccc2N(c2ccc(-c3ccccc3)cc2)c2ccc3ccccc3c2)-c2c(-c3ccc4ccccc4c3)ccc3cccc1c23]

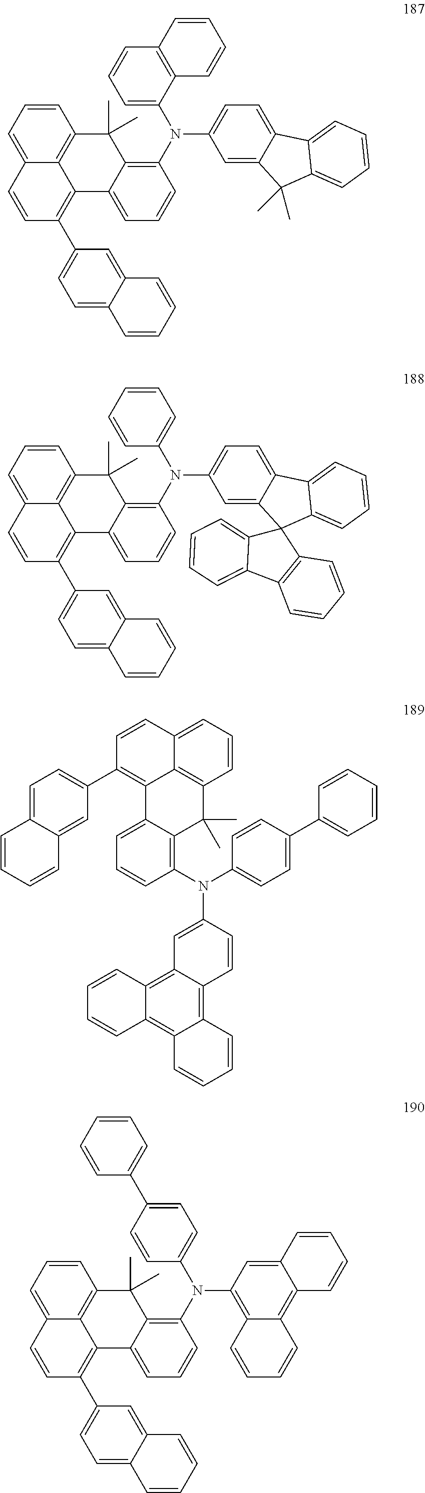

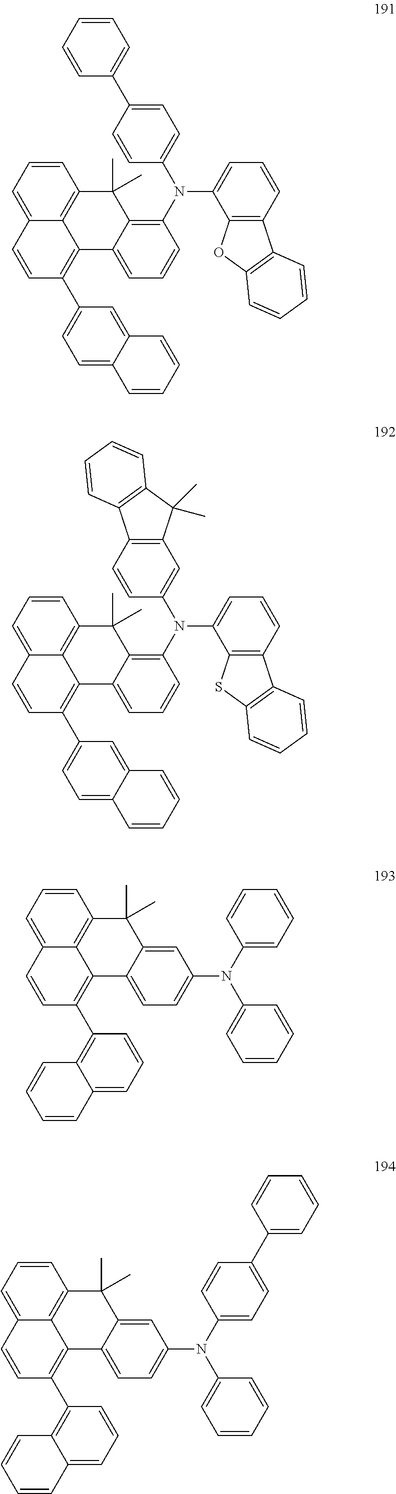 [SMILES: CC1(C)c2c(cccc2N(c2ccc(-c3ccccc3)cc2)c2cccc3c2oc2ccccc23)-c2c(-c3ccc4ccccc4c3)ccc3cccc1c23 ; CC1(C)c2ccccc2-c2ccc(N(c3cccc4c3C(C)(C)c3cccc5ccc(-c6ccc7ccccc7c6)c-4c35)c3cccc4c3sc3ccccc34)cc21 ; CC1(C)c2cc(N(c3ccccc3)c3ccccc3)ccc2-c2c(-c3cccc4ccccc34)ccc3cccc1c23 ; CC1(C)c2cc(N(c3ccccc3)c3ccc(-c4ccccc4)cc3)ccc2-c2c(-c3cccc4ccccc34)ccc3cccc1c23]

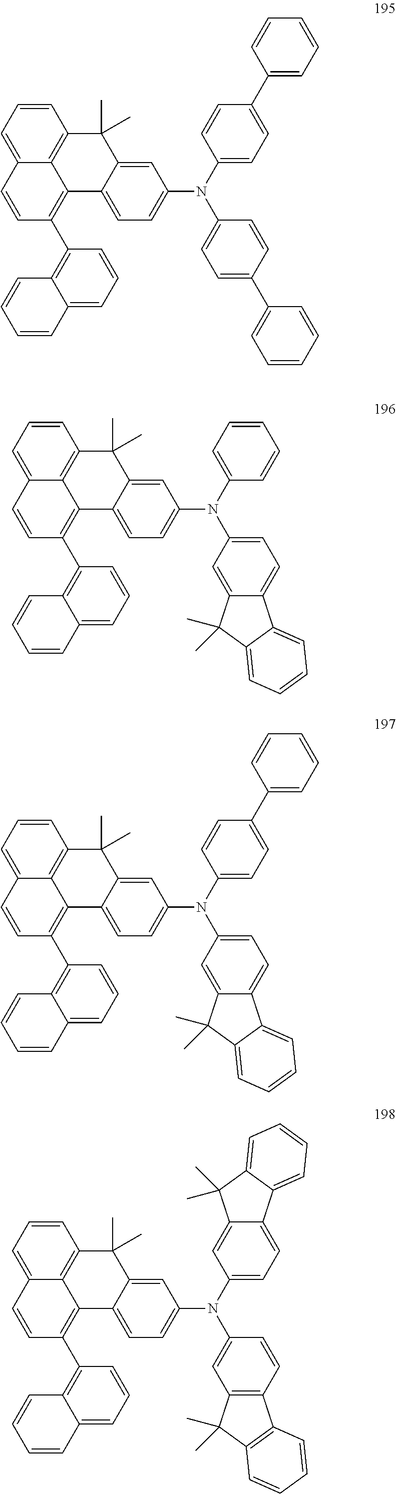

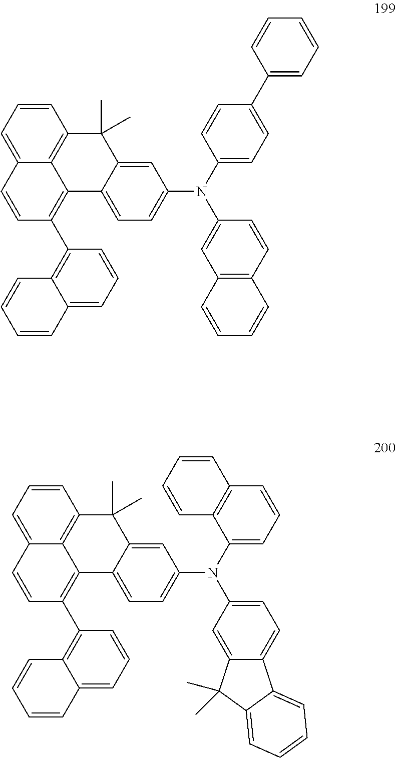 [SMILES: CC1(C)c2cc(N(c3ccc(-c4ccccc4)cc3)c3ccc4ccccc4c3)ccc2-c2c(-c3cccc4ccccc34)ccc3cccc1c23 ; CC1(C)c2ccccc2-c2ccc(N(c3ccc4c(c3)C(C)(C)c3cccc5ccc(-c6cccc7ccccc67)c-4c35)c3cccc4ccccc34)cc21]

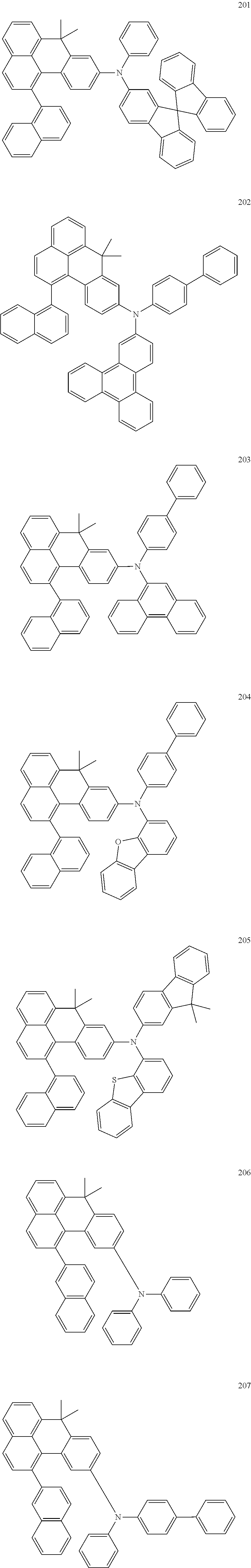

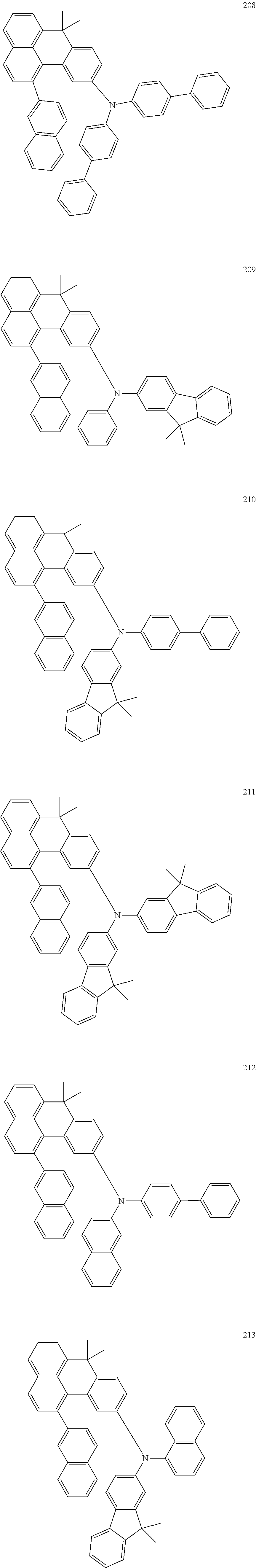

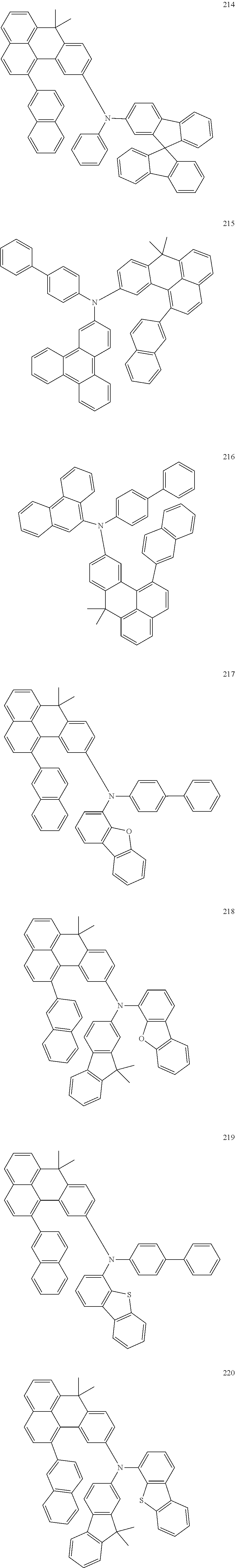

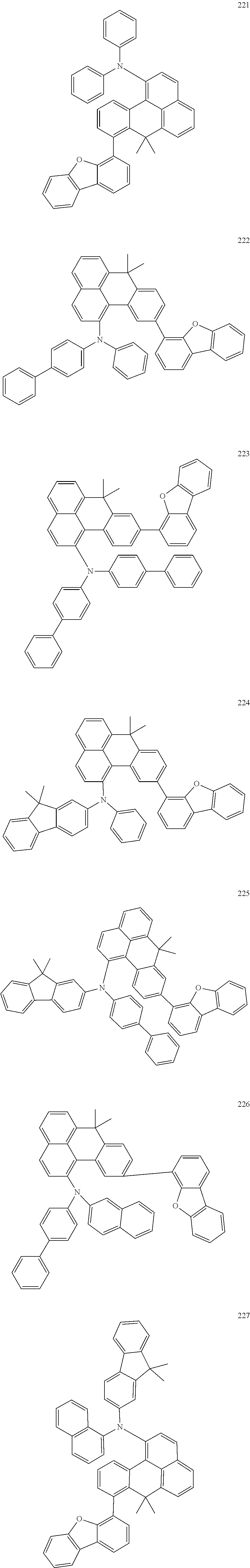

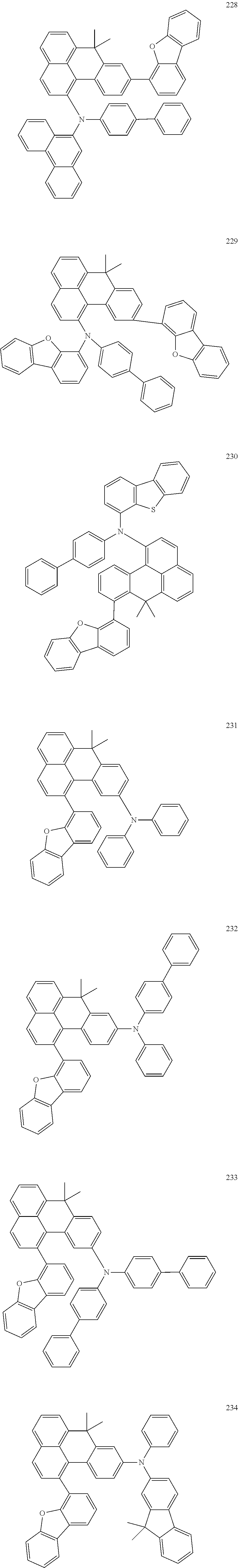

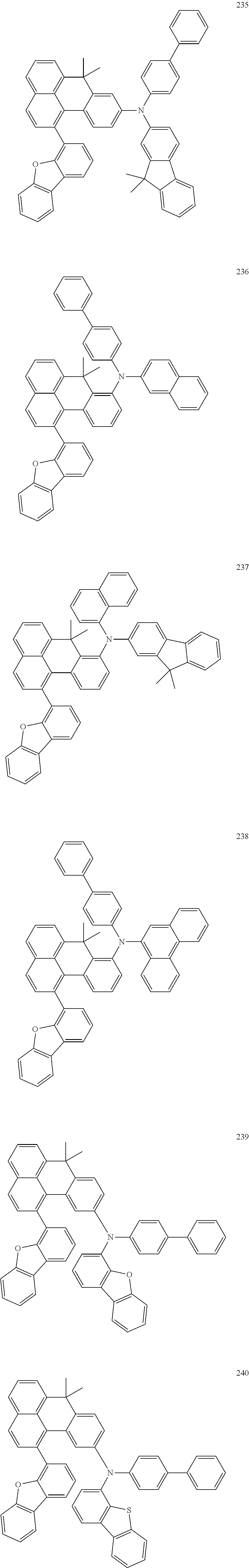

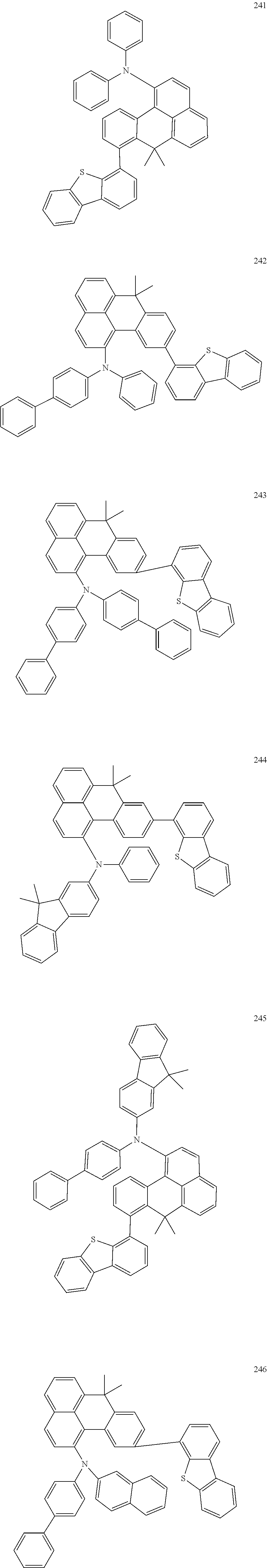

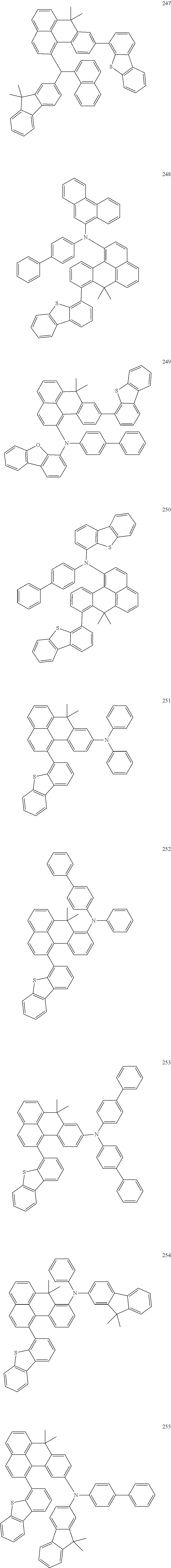

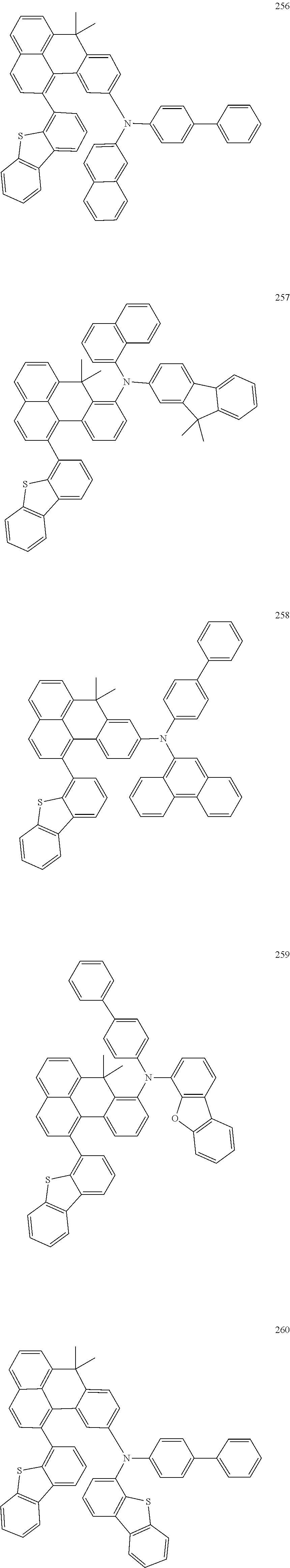 [SMILES: CC1(C)c2ccc(N(c3ccc(-c4ccccc4)cc3)c3ccc4ccccc4c3)cc2-c2c(-c3cccc4c3sc3ccccc34)ccc3cccc1c23 ; CC1(C)c2ccccc2-c2ccc(N(c3cccc4c3C(C)(C)c3cccc5ccc(-c6cccc7c6sc6ccccc67)c-4c35)c3cccc4ccccc34)cc21 ; CC1(C)c2cc(N(c3ccc(-c4ccccc4)cc3)c3cc4ccccc4c4ccccc34)ccc2-c2c(-c3cccc4c3sc3ccccc34)ccc3cccc1c23 ; CC1(C)c2c(cccc2N(c2ccc(-c3ccccc3)cc2)c2cccc3c2oc2ccccc23)-c2c(-c3cccc4c3sc3ccccc34)ccc3cccc1c23 ; CC1(C)c2ccc(N(c3ccc(-c4ccccc4)cc3)c3cccc4c3sc3ccccc34)cc2-c2c(-c3cccc4c3sc3ccccc34)ccc3cccc1c23]

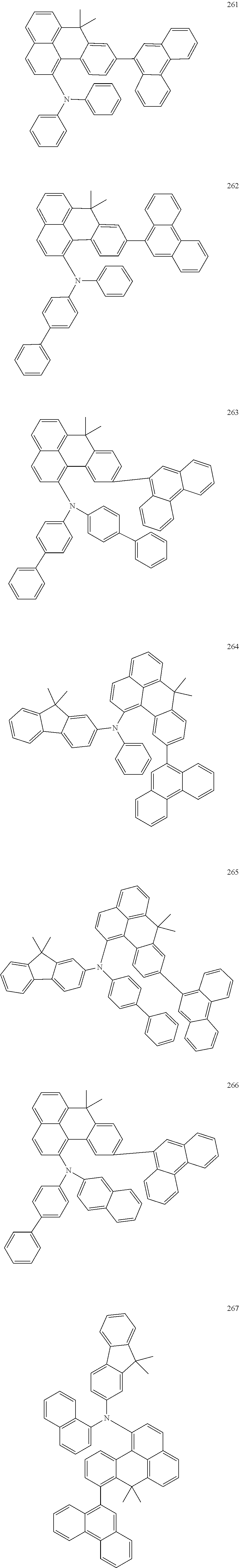

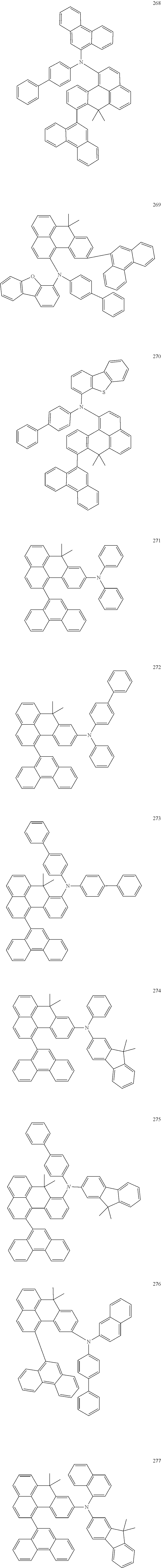

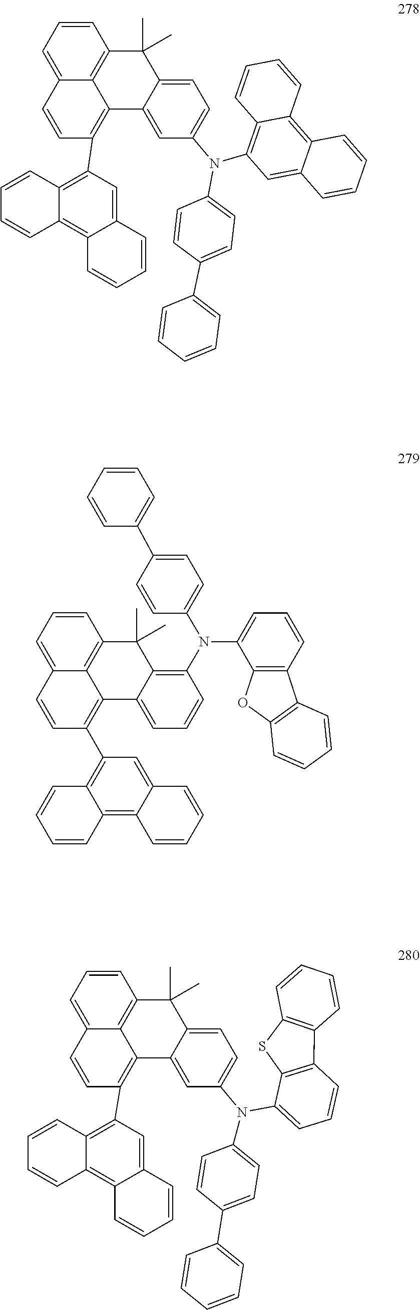 [SMILES: CC1(C)c2ccc(N(c3ccc(-c4ccccc4)cc3)c3cc4ccccc4c4ccccc34)cc2-c2c(-c3cc4ccccc4c4ccccc34)ccc3cccc1c23 ; CC1(C)c2c(cccc2N(c2ccc(-c3ccccc3)cc2)c2cccc3c2oc2ccccc23)-c2c(-c3cc4ccccc4c4ccccc34)ccc3cccc1c23 ; CC1(C)c2ccc(N(c3ccc(-c4ccccc4)cc3)c3cccc4c3sc3ccccc34)cc2-c2c(-c3cc4ccccc4c4ccccc34)ccc3cccc1c23]

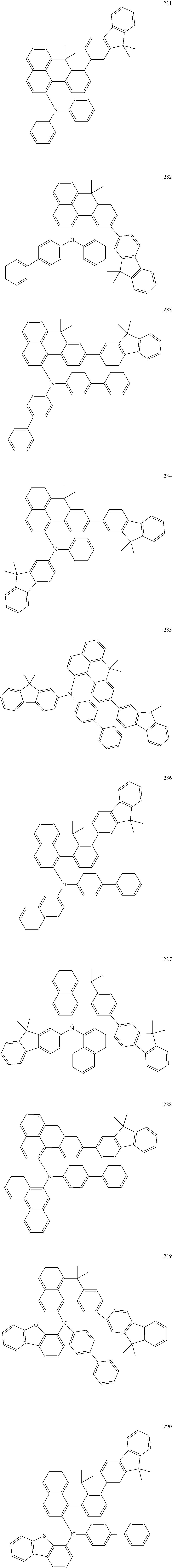

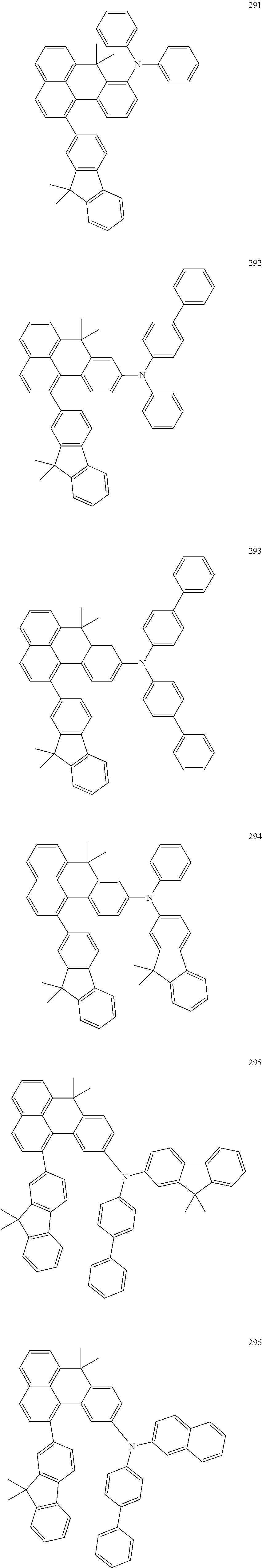

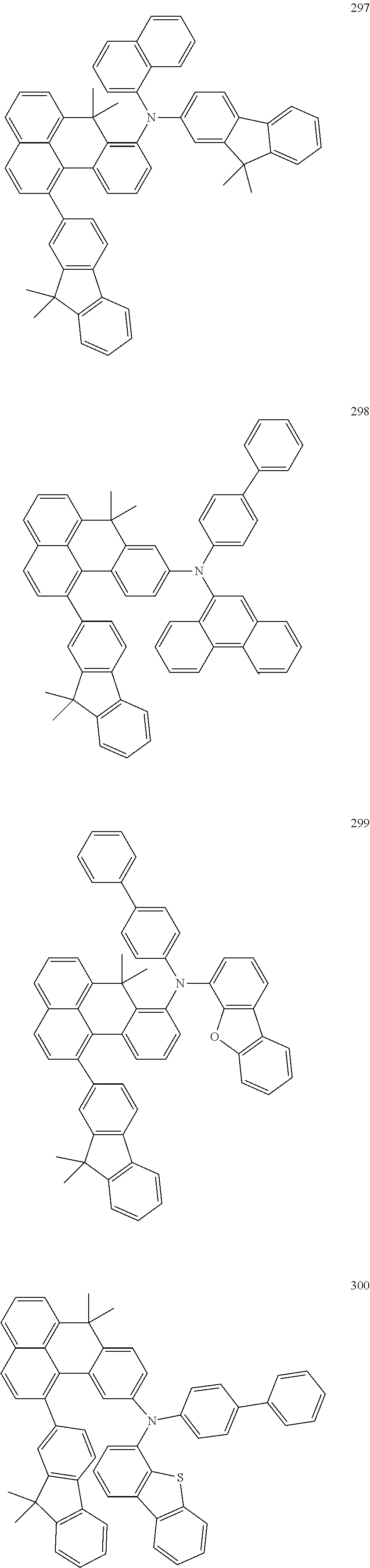 [SMILES: CC1(C)c2ccccc2-c2ccc(-c3ccc4cccc5c4c3-c3cccc(N(c4ccc6c(c4)C(C)(C)c4ccccc4-6)c4cccc6ccccc46)c3C5(C)C)cc21 ; Cc1cccc2cc(N(c3ccc(-c4ccccc4)cc3)c3ccc4c(c3)C(C)(C)c3cccc5ccc(-c6ccc7c(c6)C(C)(C)c6ccccc6-7)c-4c35)c3ccccc3c12 ; CC1(C)c2ccccc2-c2ccc(-c3ccc4cccc5c4c3-c3cccc(N(c4ccc(-c6ccccc6)cc4)c4cccc6c4oc4ccccc46)c3C5(C)C)cc21 ; CC1(C)c2ccccc2-c2ccc(-c3ccc4cccc5c4c3-c3cc(N(c4ccc(-c6ccccc6)cc4)c4cccc6c4sc4ccccc46)ccc3C5(C)C)cc21]

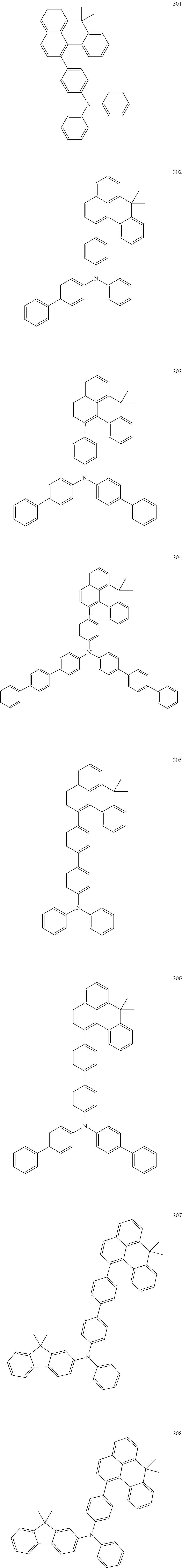

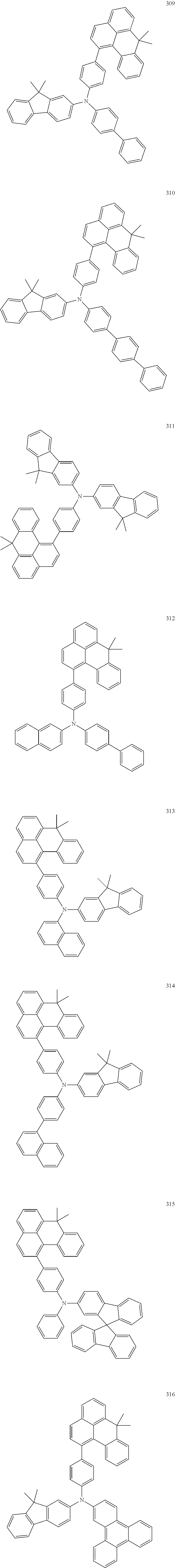

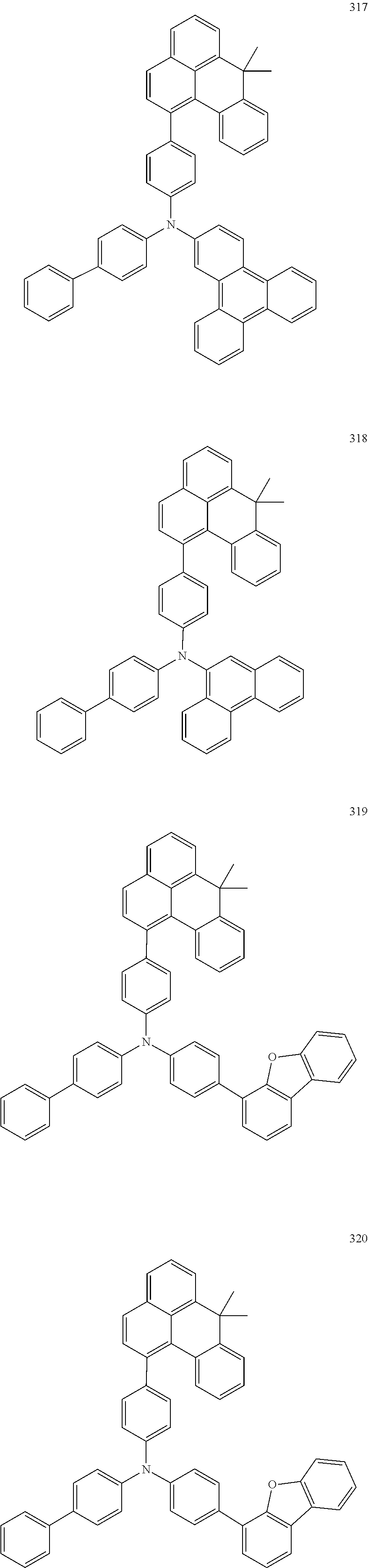 [SMILES: CC1(C)c2ccccc2-c2c(-c3ccc(N(c4ccc(-c5ccccc5)cc4)c4ccc5c6ccccc6c6ccccc6c5c4)cc3)ccc3cccc1c23 ; CC1(C)c2ccccc2-c2c(-c3ccc(N(c4ccc(-c5ccccc5)cc4)c4cc5ccccc5c5ccccc45)cc3)ccc3cccc1c23 ; CC1(C)c2ccccc2-c2c(-c3ccc(N(c4ccc(-c5ccccc5)cc4)c4ccc(-c5cccc6c5oc5ccccc56)cc4)cc3)ccc3cccc1c23 ; CC1(C)c2ccccc2-c2c(-c3ccc(N(c4ccc(-c5ccccc5)cc4)c4ccc(-c5cccc6c5oc5ccccc56)cc4)cc3)ccc3cccc1c23]

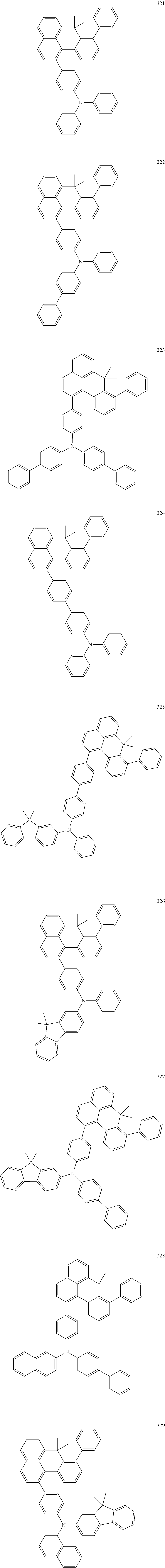

[chemical structure]

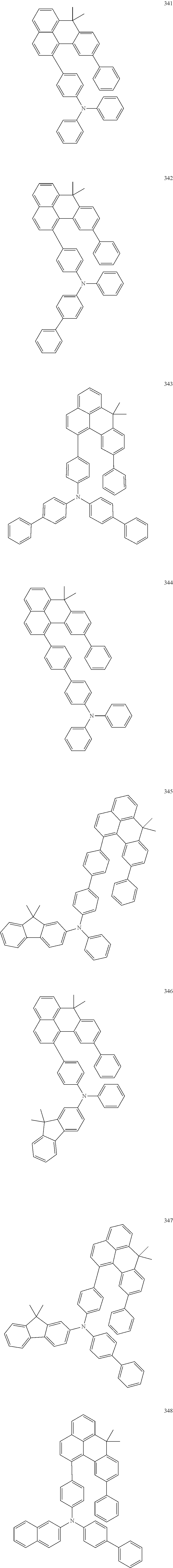

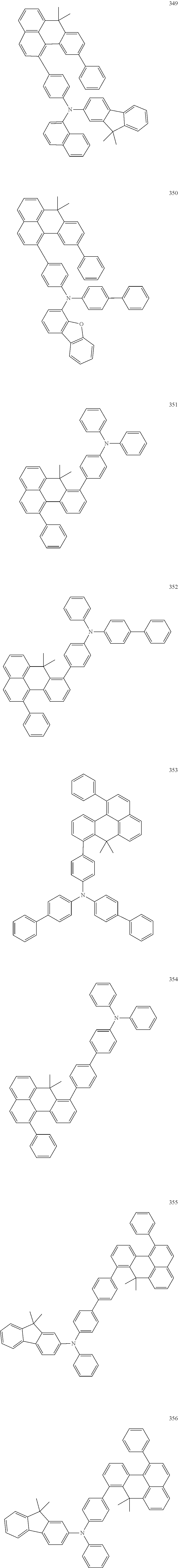

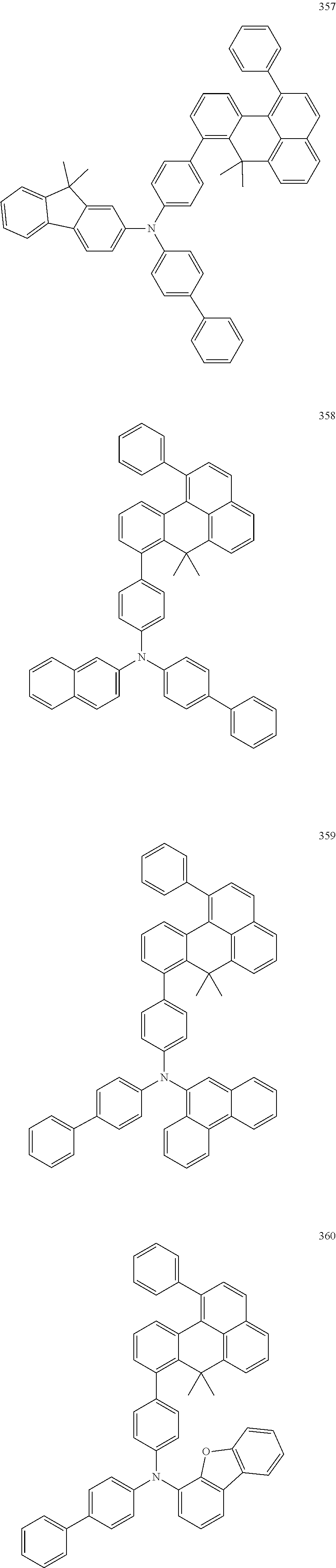 [SMILES: CC1(C)c2ccccc2-c2ccc(N(c3ccc(-c4ccccc4)cc3)c3ccc(-c4cccc5c4C(C)(C)c4cccc6ccc(-c7ccccc7)c-5c46)cc3)cc21 ; CC1(C)c2c(-c3ccc(N(c4ccc(-c5ccccc5)cc4)c4ccc5ccccc5c4)cc3)cccc2-c2c(-c3ccccc3)ccc3cccc1c23 ; CC1(C)c2c(-c3ccc(N(c4ccc(-c5ccccc5)cc4)c4cc5ccccc5c5ccccc45)cc3)cccc2-c2c(-c3ccccc3)ccc3cccc1c23 ; CC1(C)c2c(-c3ccc(N(c4ccc(-c5ccccc5)cc4)c4cccc5c4oc4ccccc45)cc3)cccc2-c2c(-c3ccccc3)ccc3cccc1c23]

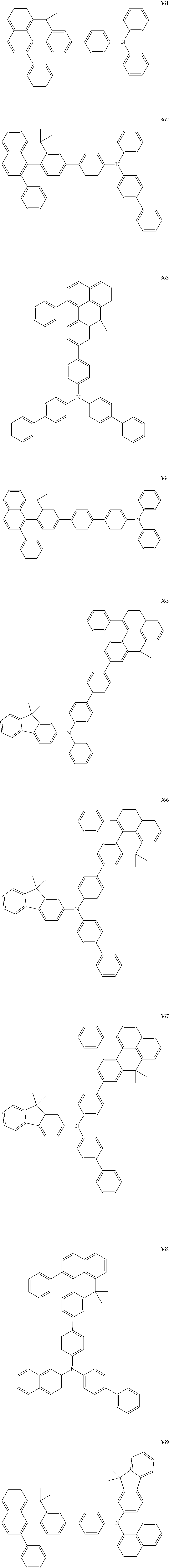

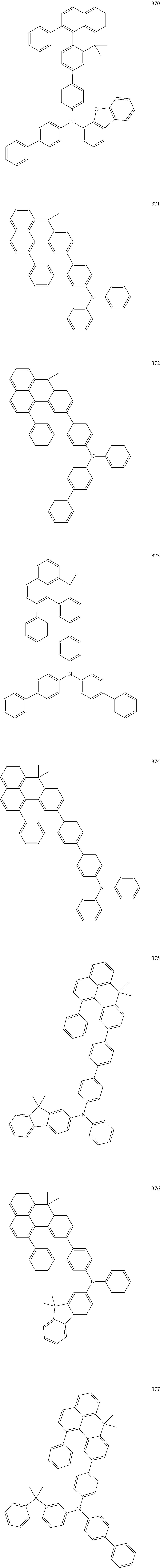

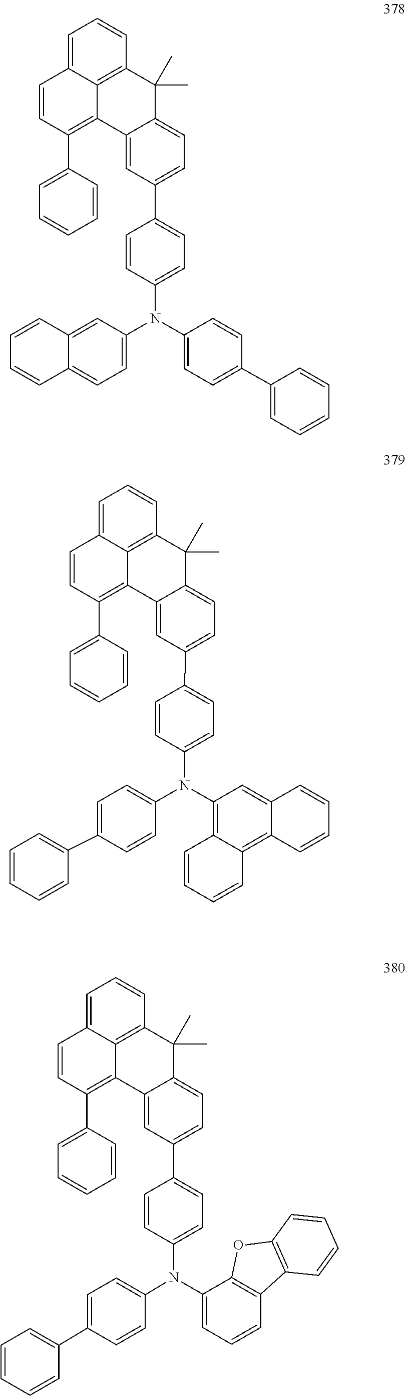 [SMILES: CC1(C)c2ccc(-c3ccc(N(c4ccc(-c5ccccc5)cc4)c4ccc5ccccc5c4)cc3)cc2-c2c(-c3ccccc3)ccc3cccc1c23 ; CC1(C)c2ccc(-c3ccc(N(c4ccc(-c5ccccc5)cc4)c4cc5ccccc5c5ccccc45)cc3)cc2-c2c(-c3ccccc3)ccc3cccc1c23 ; CC1(C)c2ccc(-c3ccc(N(c4ccc(-c5ccccc5)cc4)c4cccc5c4oc4ccccc45)cc3)cc2-c2c(-c3ccccc3)ccc3cccc1c23]

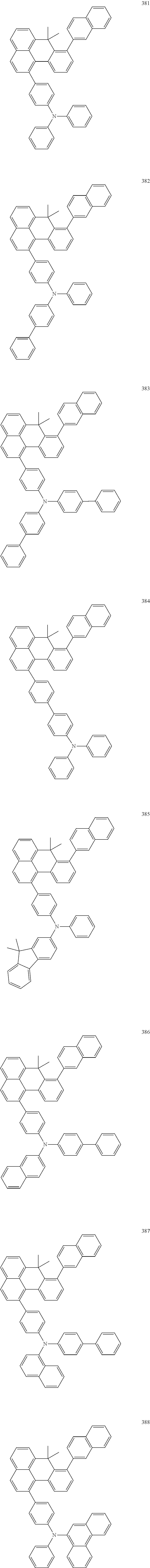

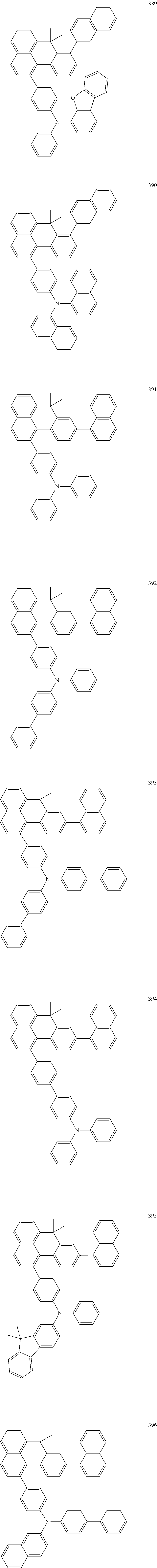

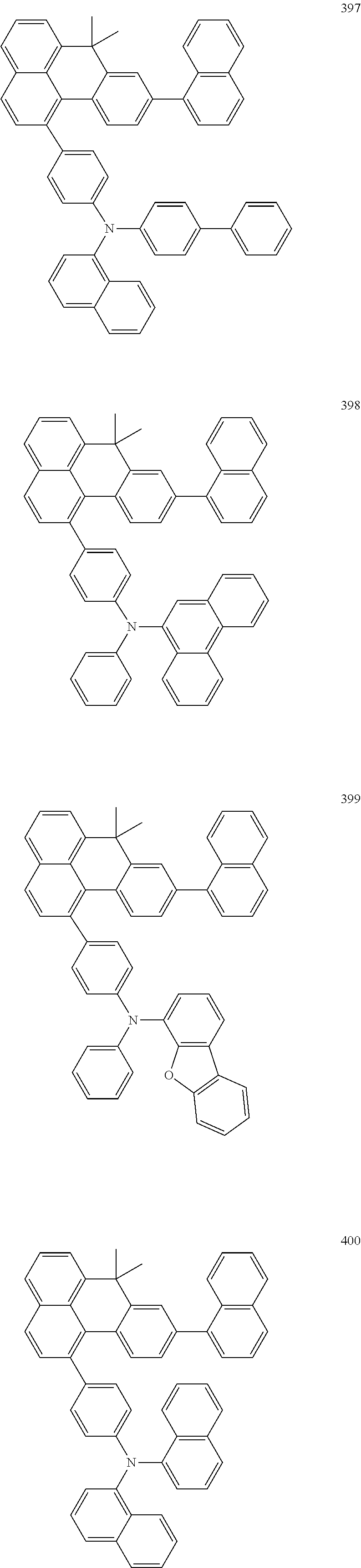 [SMILES: CC1(C)c2cc(-c3cccc4ccccc34)ccc2-c2c(-c3ccc(N(c4ccc(-c5ccccc5)cc4)c4cccc5ccccc45)cc3)ccc3cccc1c23 ; CC1(C)c2cc(-c3cccc4ccccc34)ccc2-c2c(-c3ccc(N(c4ccccc4)c4cc5ccccc5c5ccccc45)cc3)ccc3cccc1c23 ; CC1(C)c2cc(-c3cccc4ccccc34)ccc2-c2c(-c3ccc(N(c4ccccc4)c4cccc5c4oc4ccccc45)cc3)ccc3cccc1c23 ; CC1(C)c2cc(-c3cccc4ccccc34)ccc2-c2c(-c3ccc(N(c4cccc5ccccc45)c4cccc5ccccc45)cc3)ccc3cccc1c23]

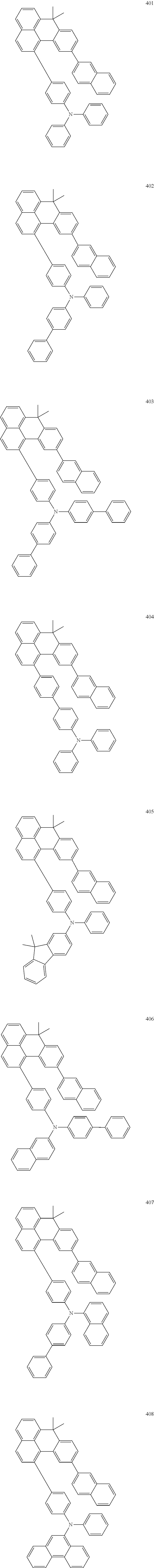

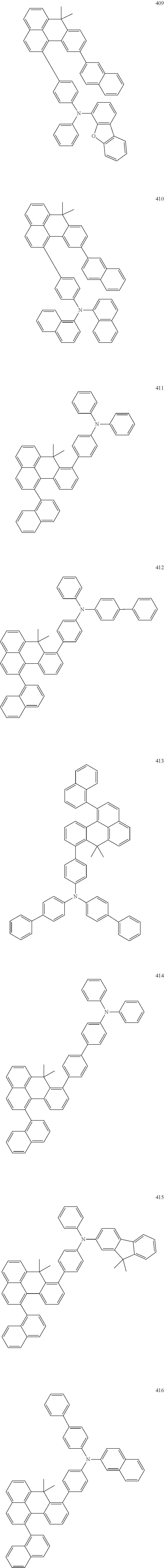

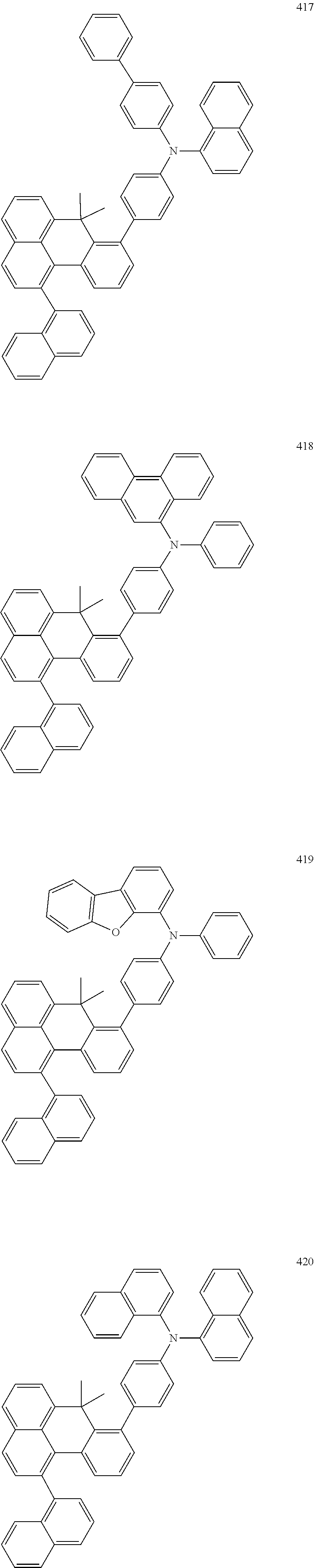 [SMILES: CC1(C)c2c(-c3ccc(N(c4ccc(-c5ccccc5)cc4)c4cccc5ccccc45)cc3)cccc2-c2c(-c3cccc4ccccc34)ccc3cccc1c23 ; CC1(C)c2c(-c3ccc(N(c4ccccc4)c4cc5ccccc5c5ccccc45)cc3)cccc2-c2c(-c3cccc4ccccc34)ccc3cccc1c23 ; CC1(C)c2c(-c3ccc(N(c4ccccc4)c4cccc5c4oc4ccccc45)cc3)cccc2-c2c(-c3cccc4ccccc34)ccc3cccc1c23 ; CC1(C)c2c(-c3ccc(N(c4cccc5ccccc45)c4cccc5ccccc45)cc3)cccc2-c2c(-c3cccc4ccccc34)ccc3cccc1c23]

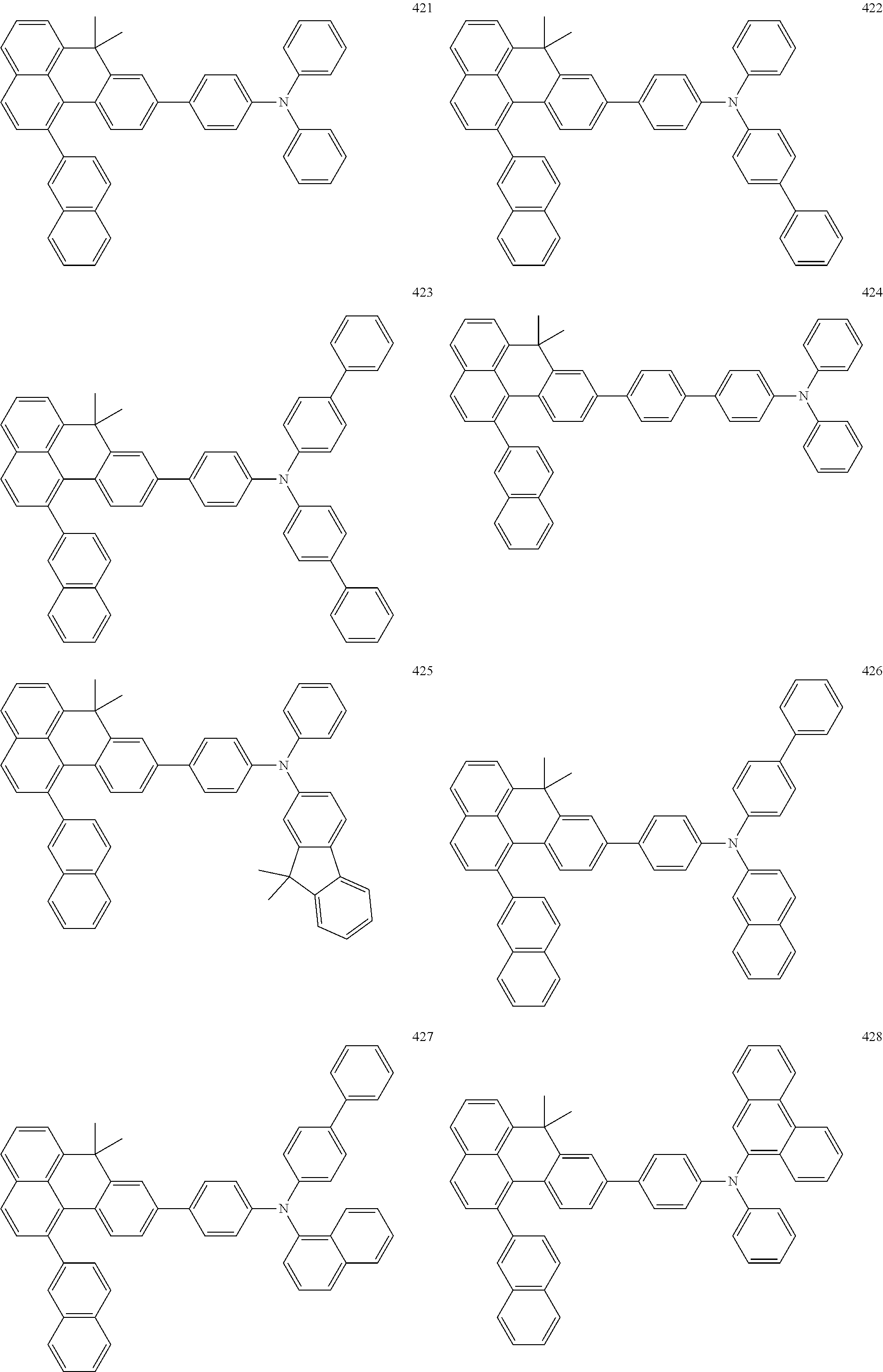

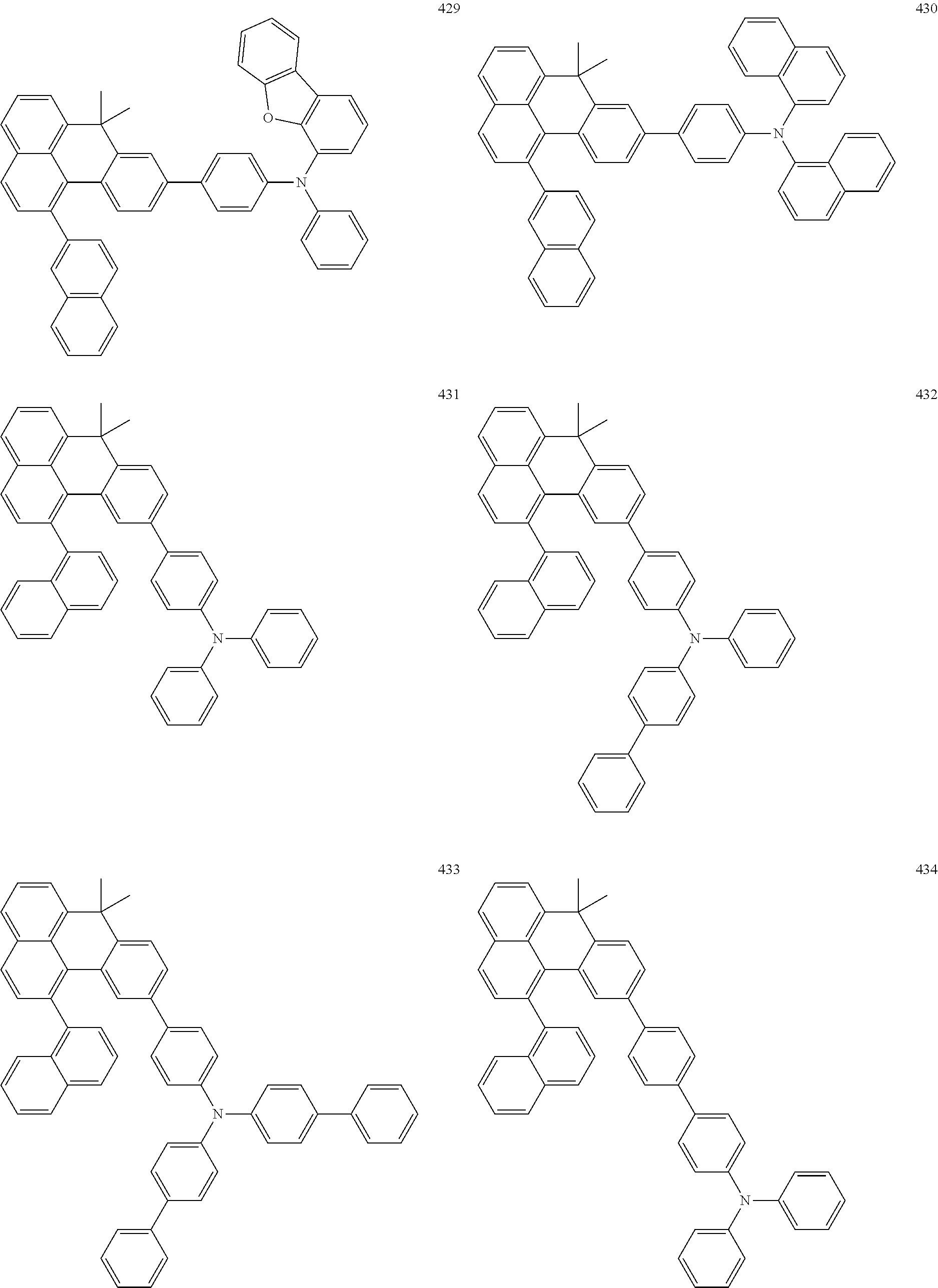

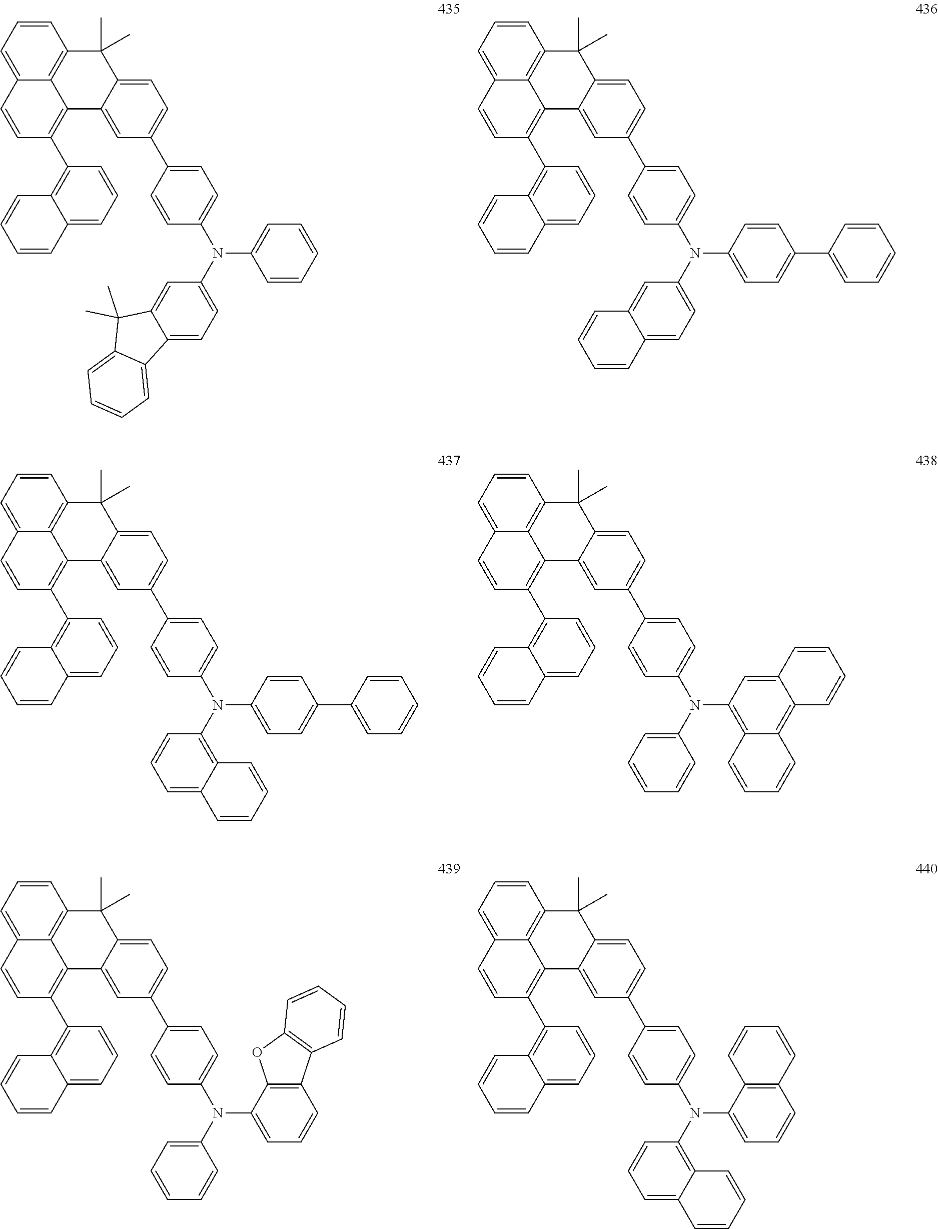

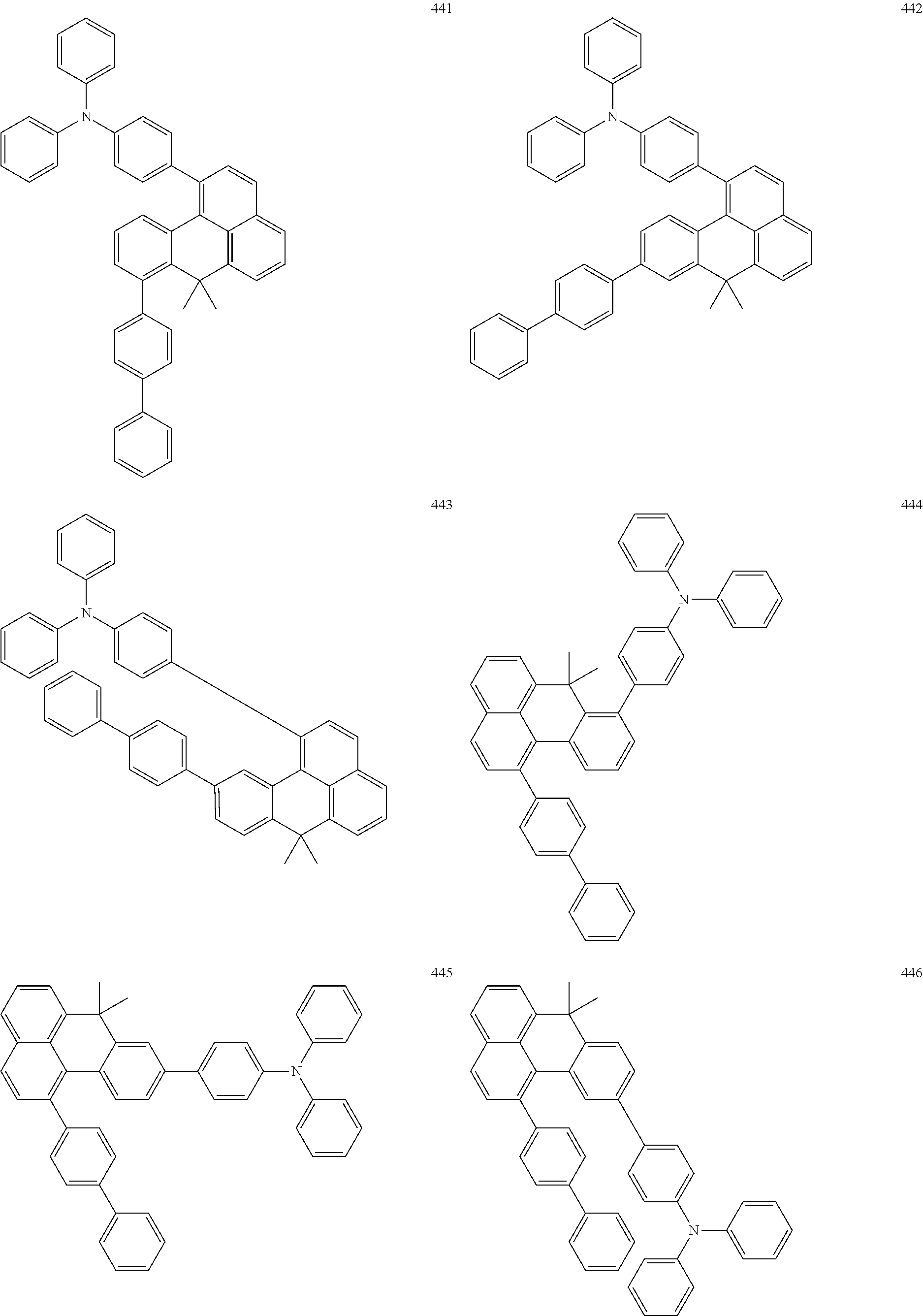

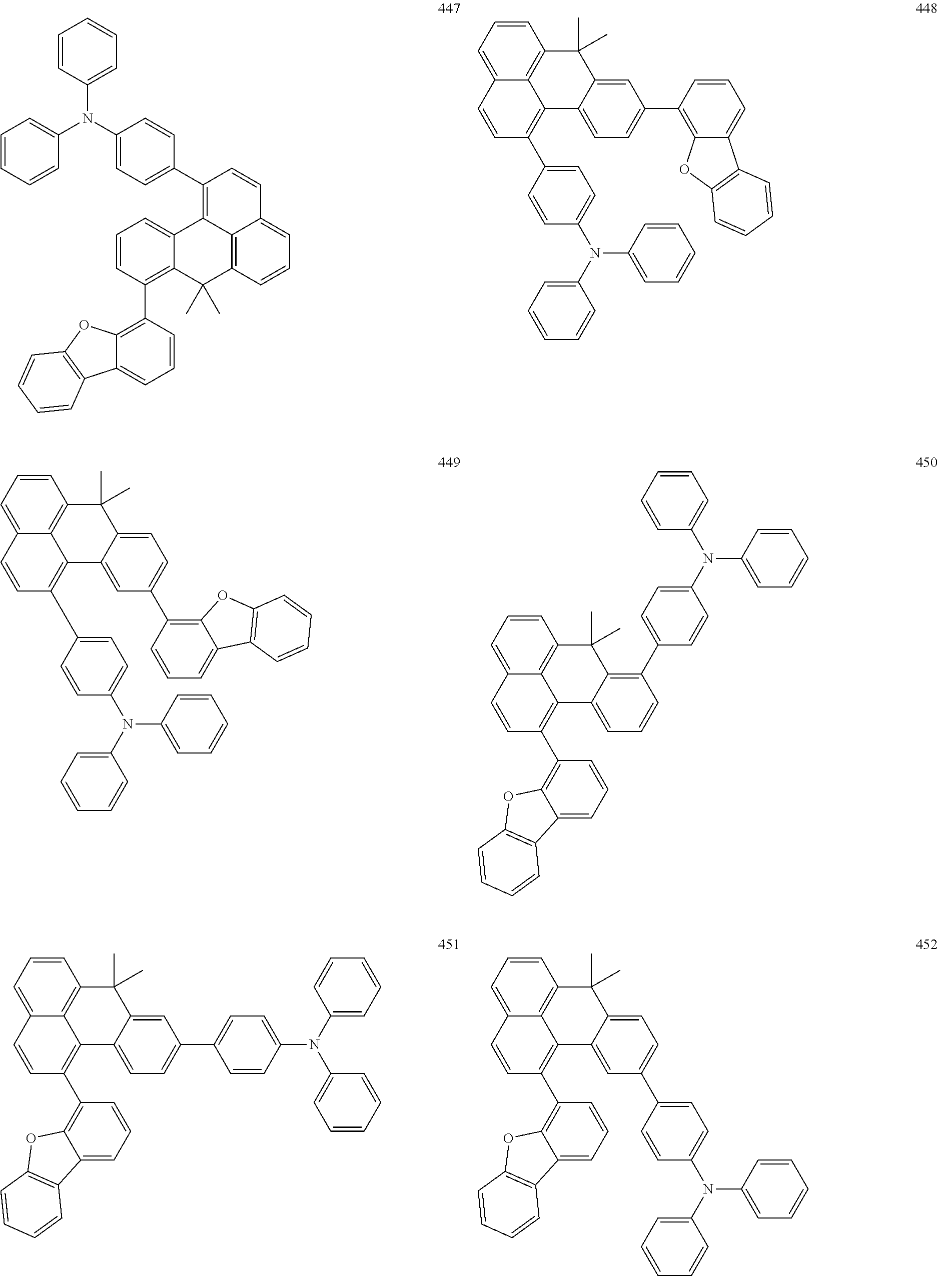

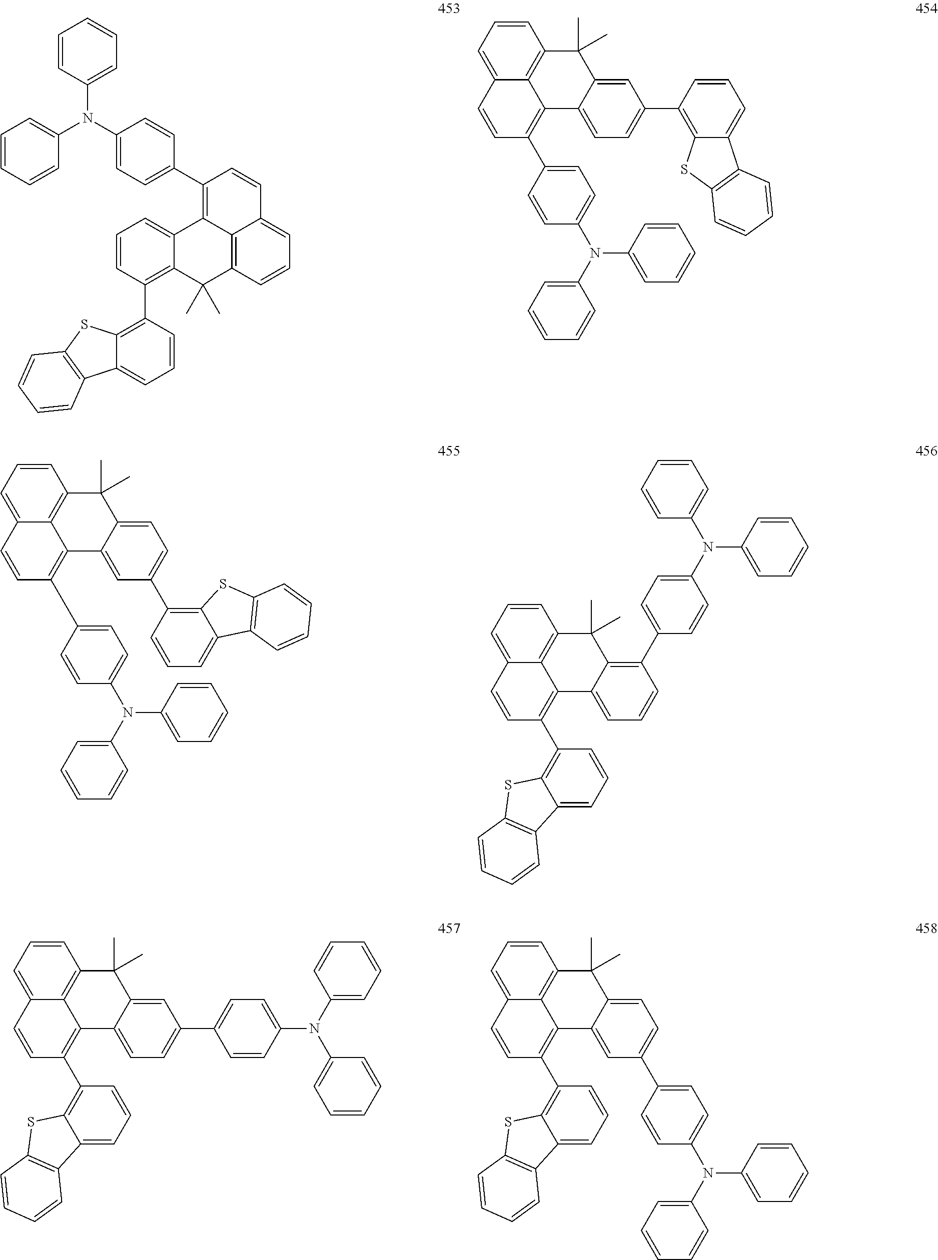

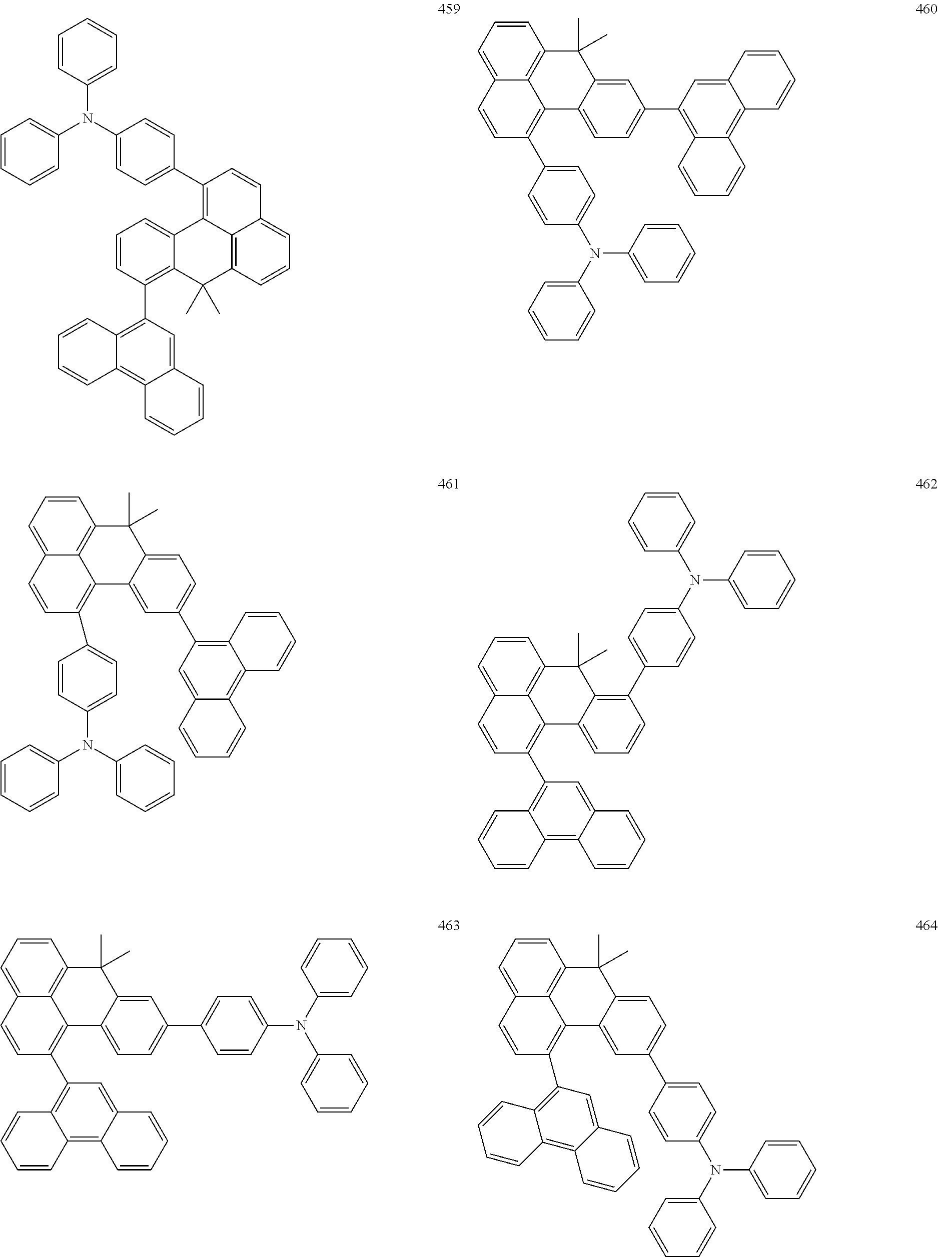

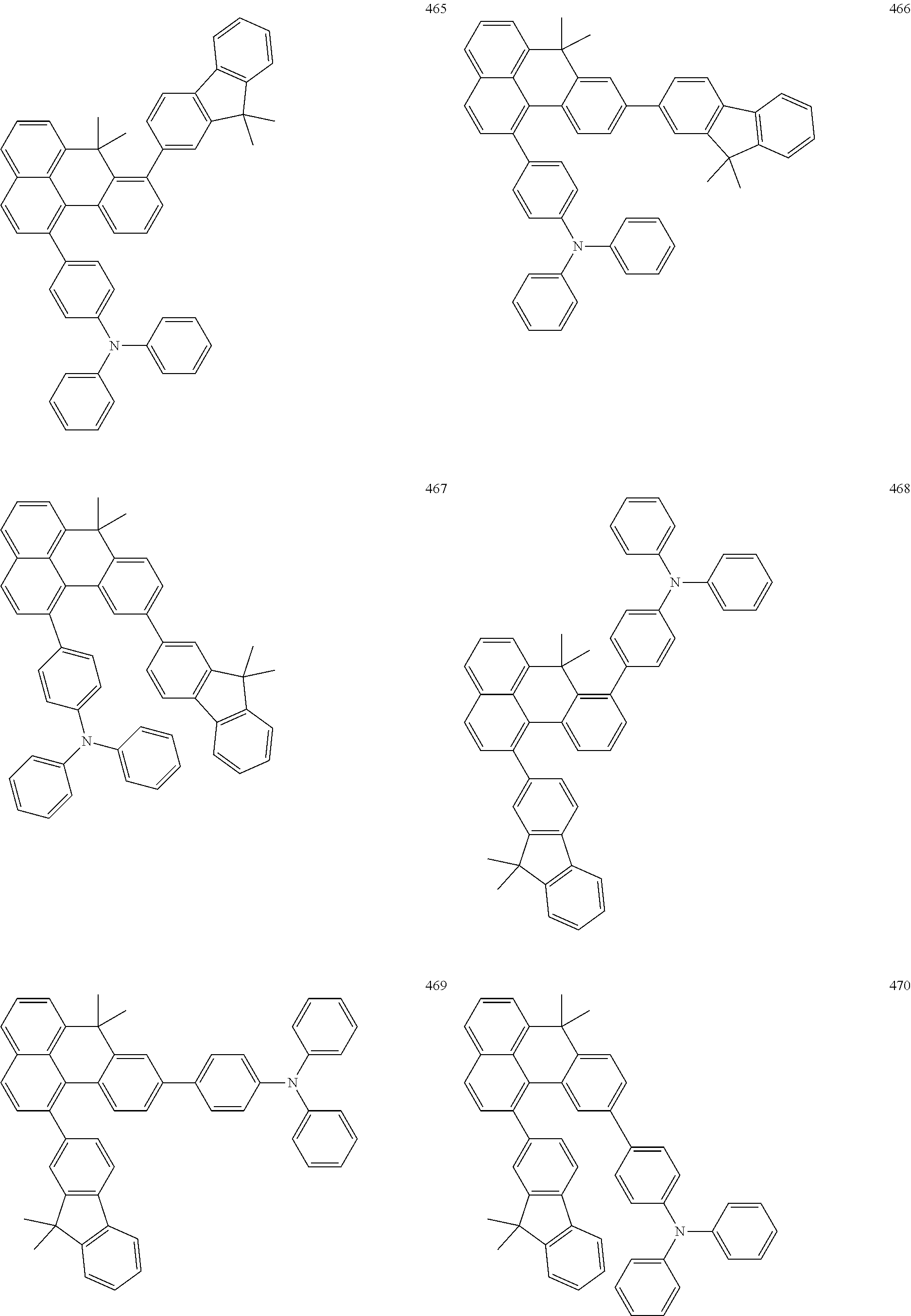

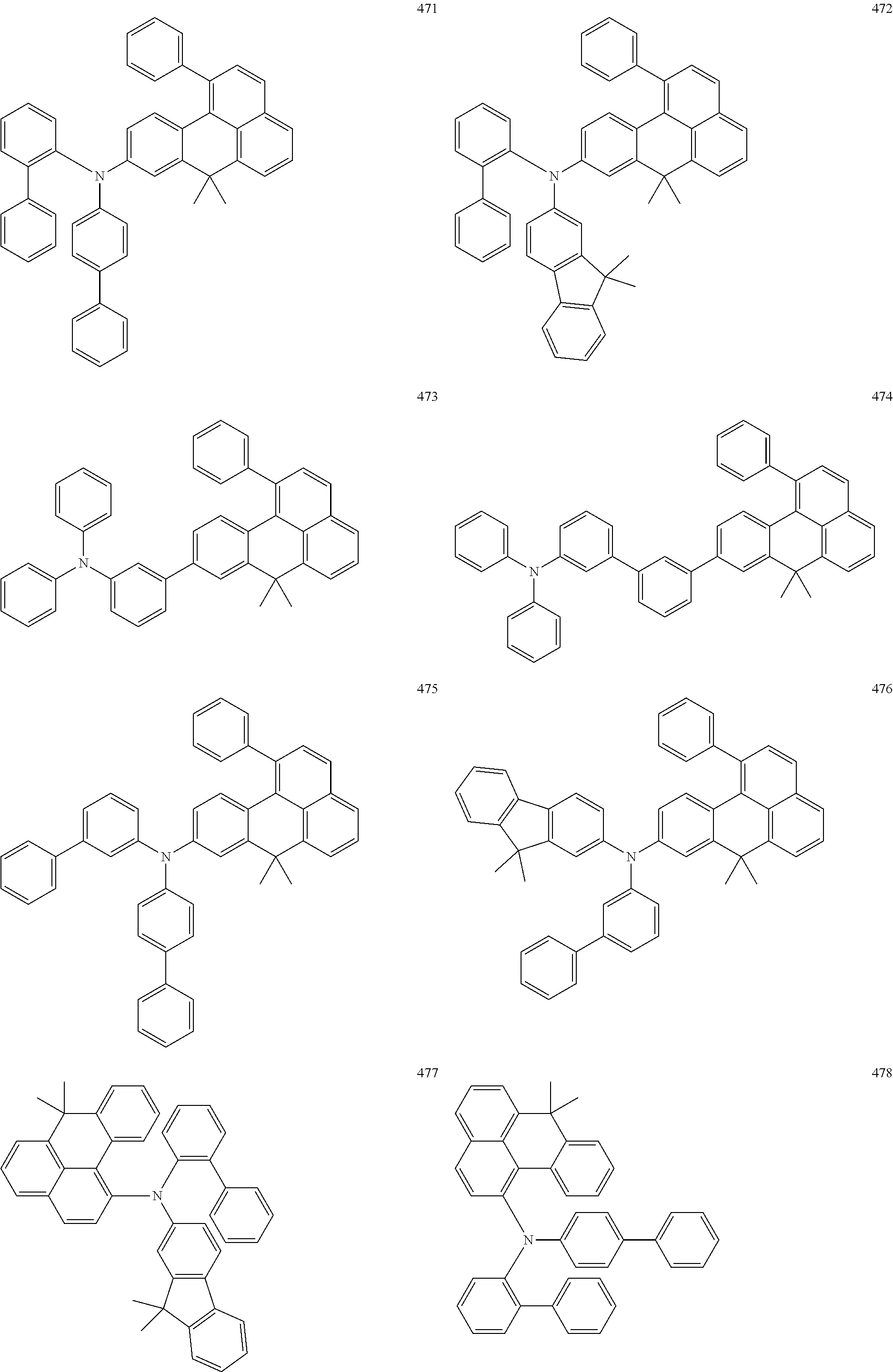

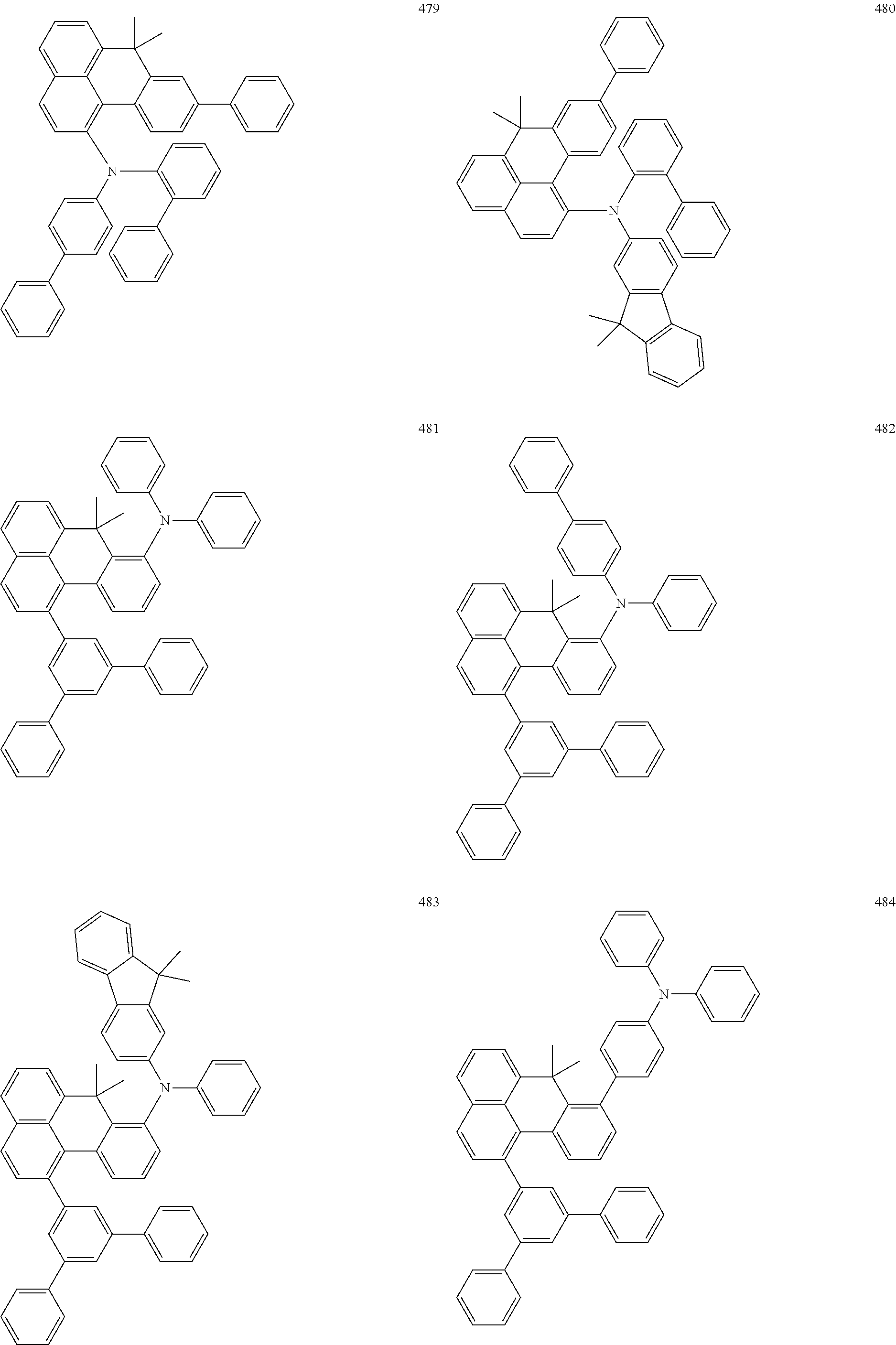

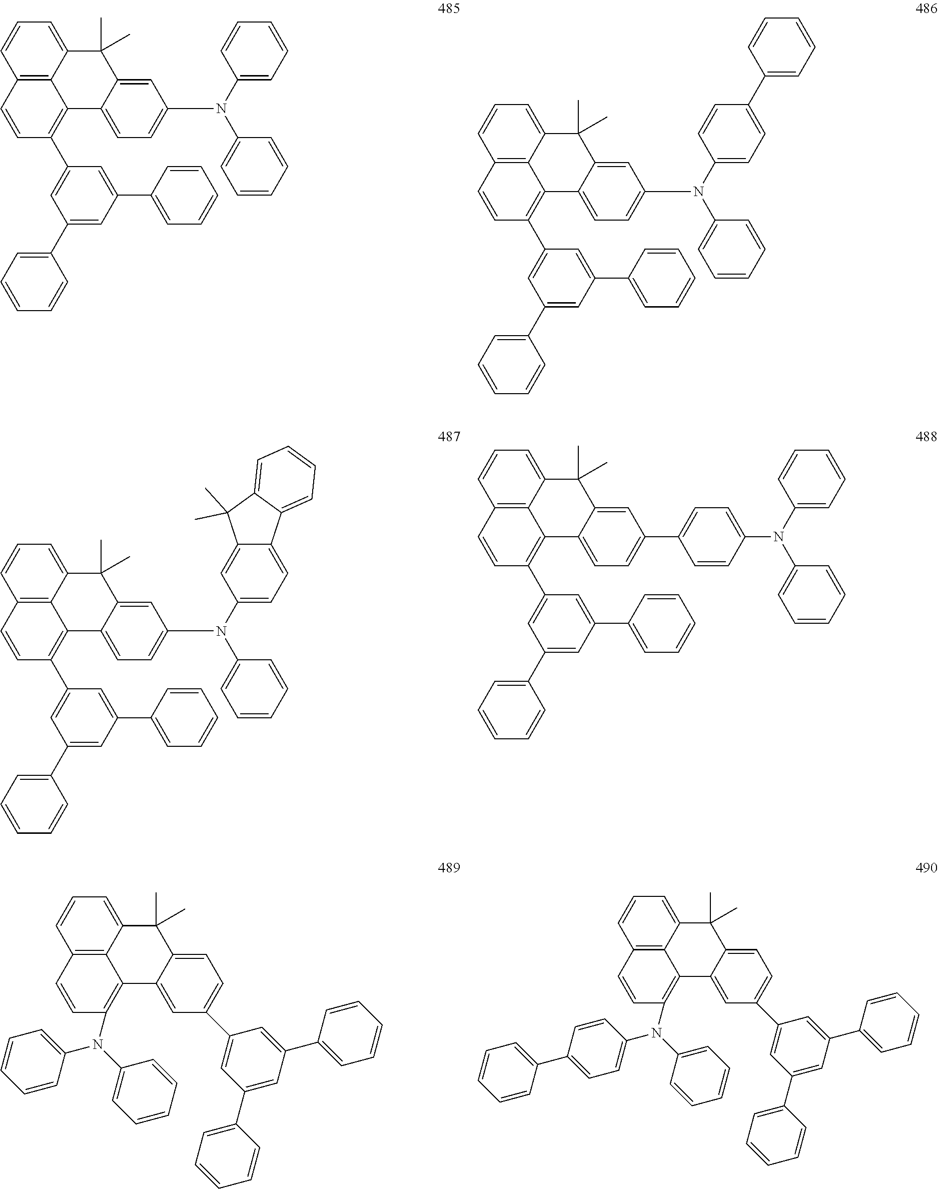

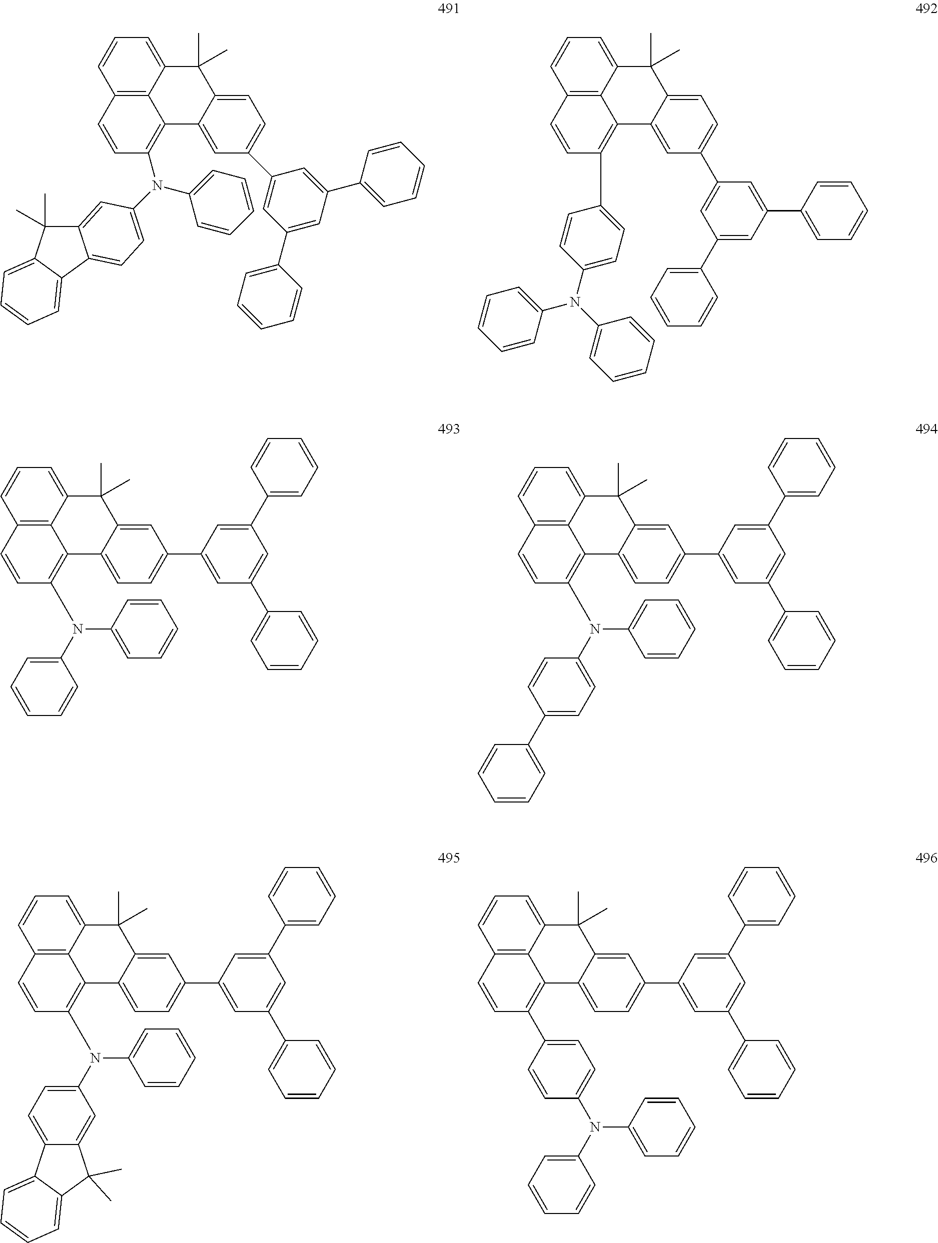

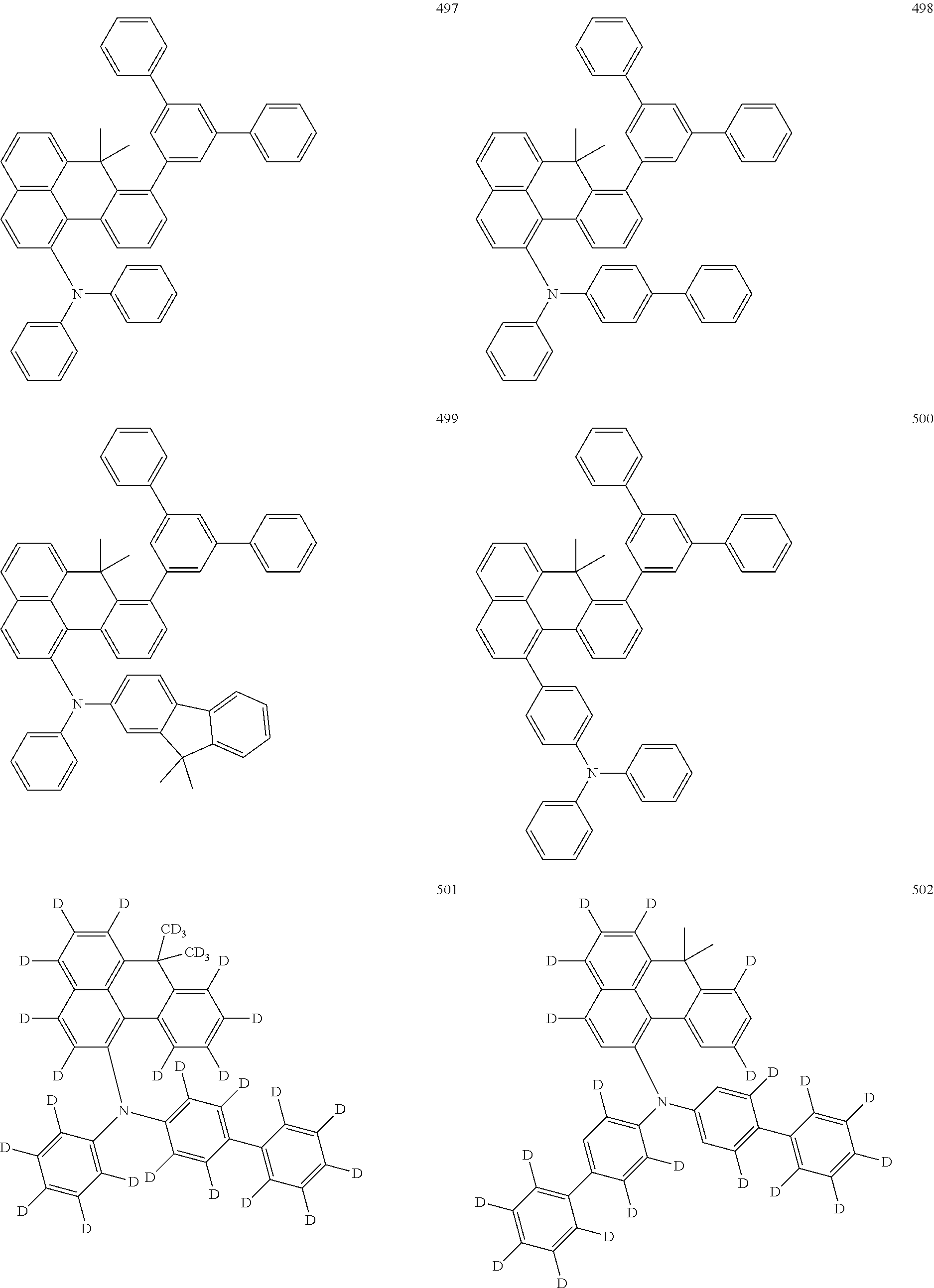

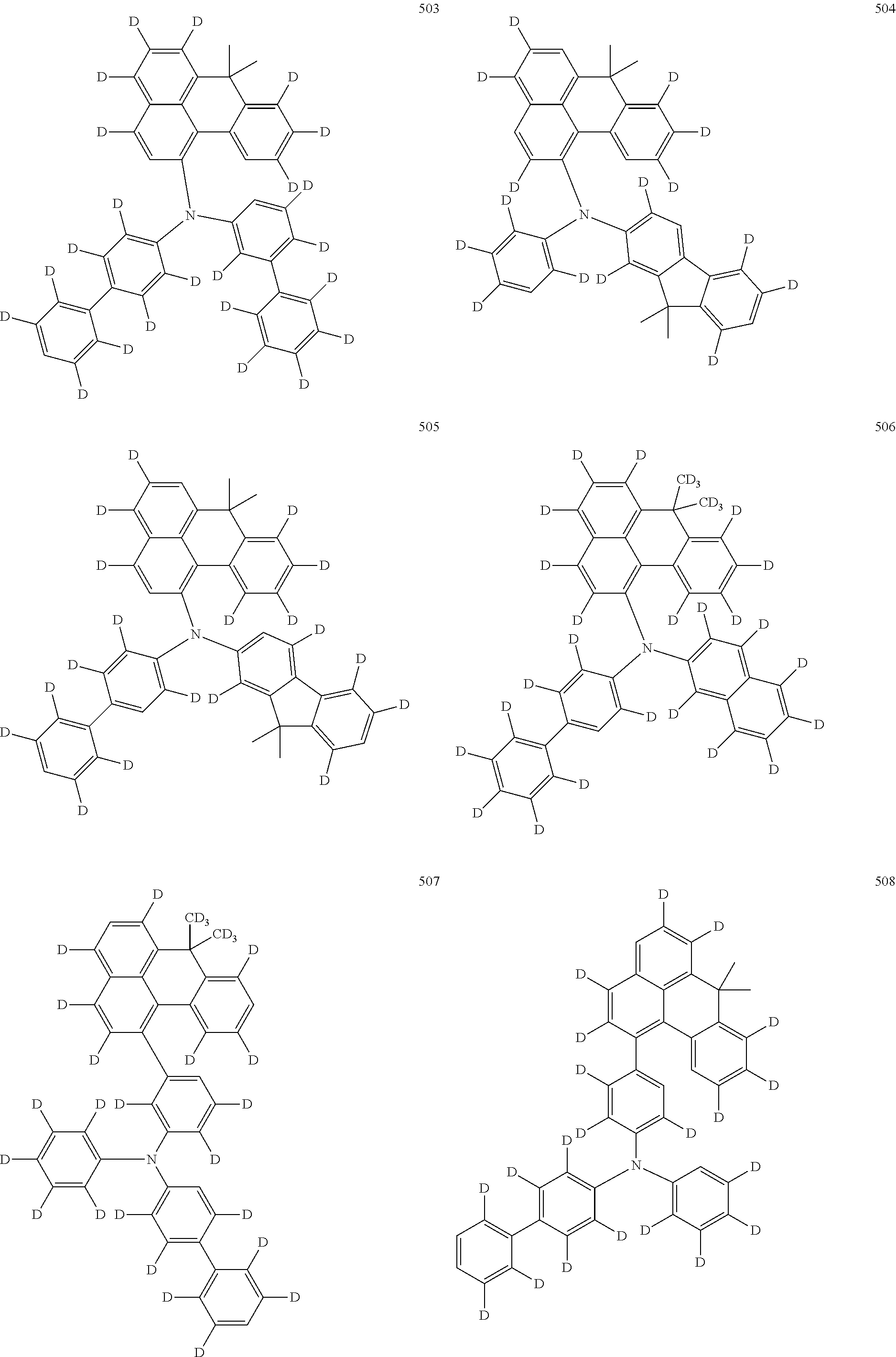

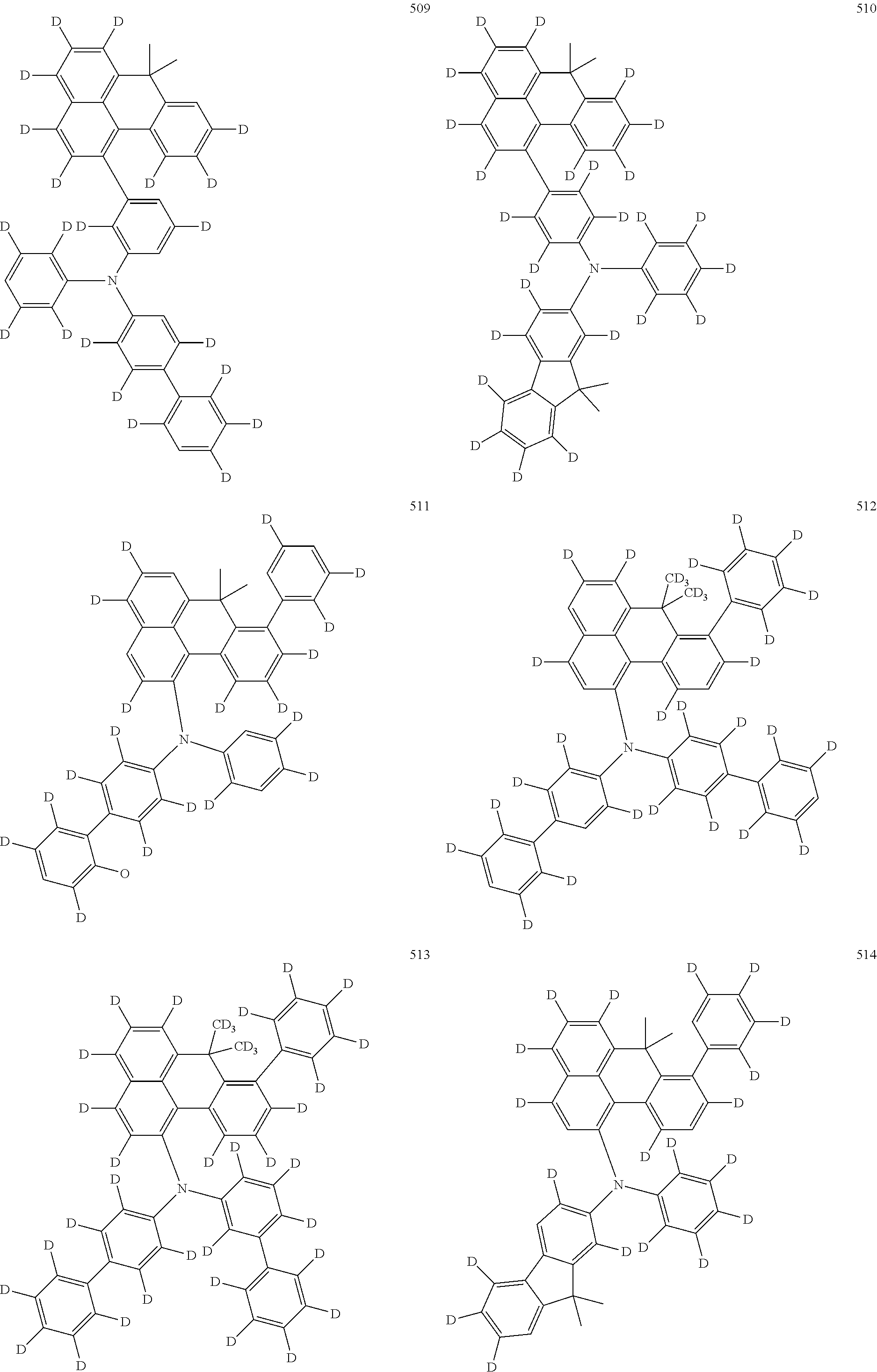

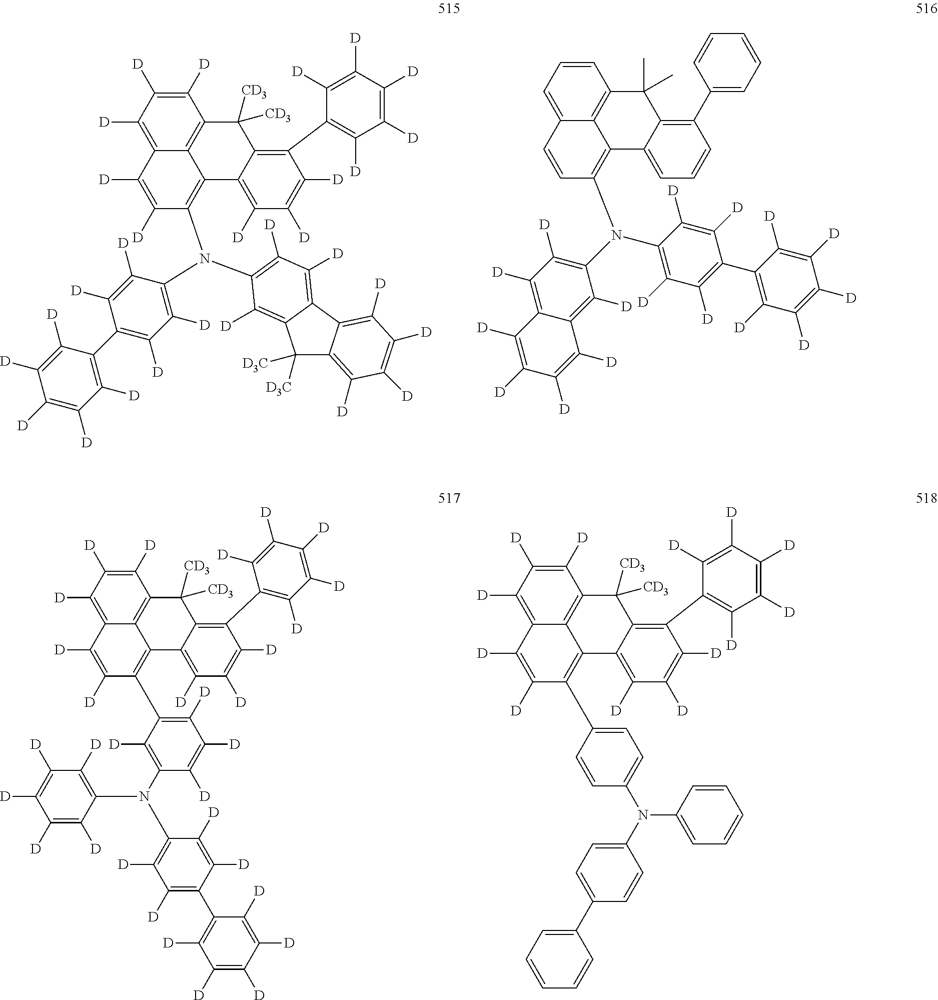

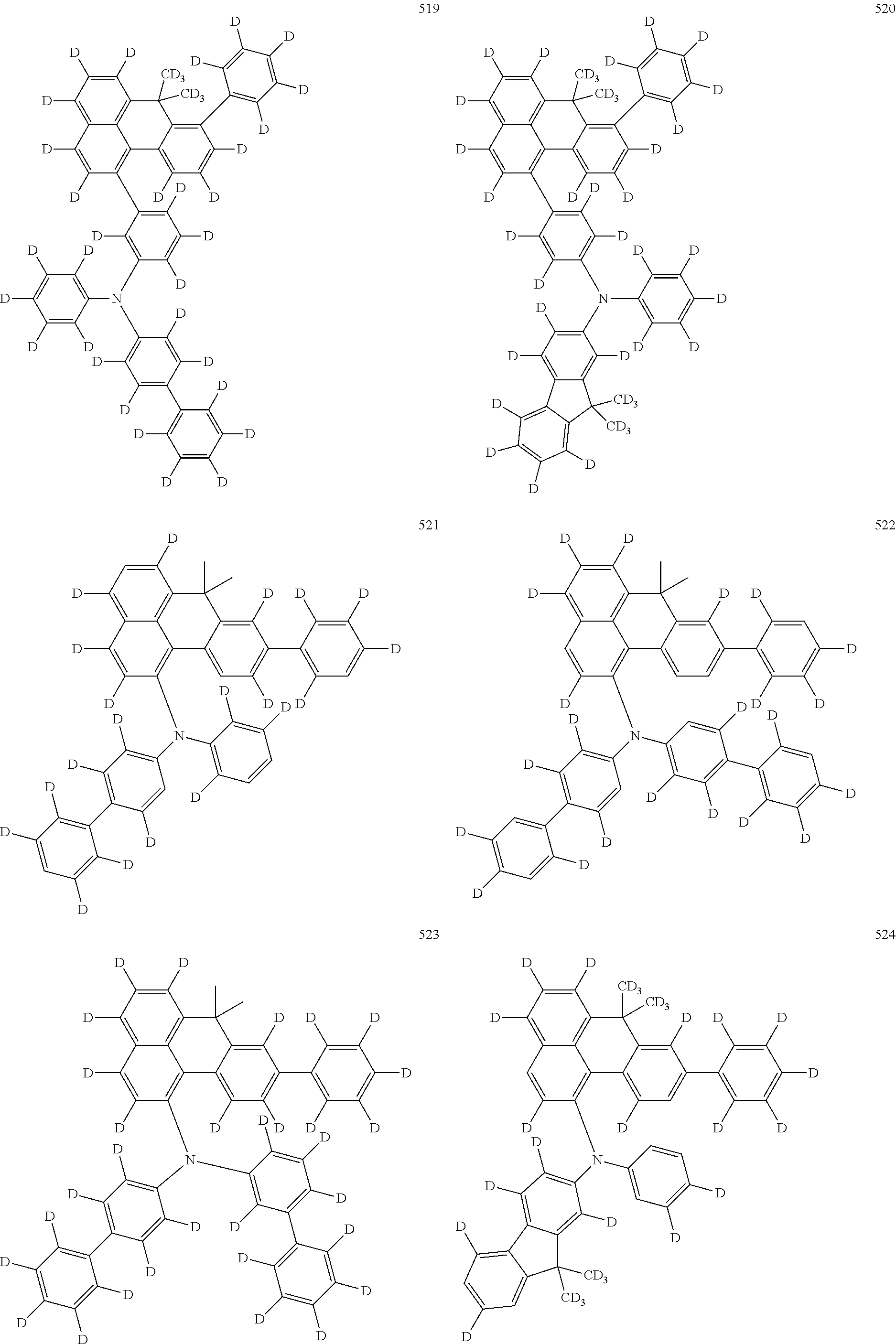

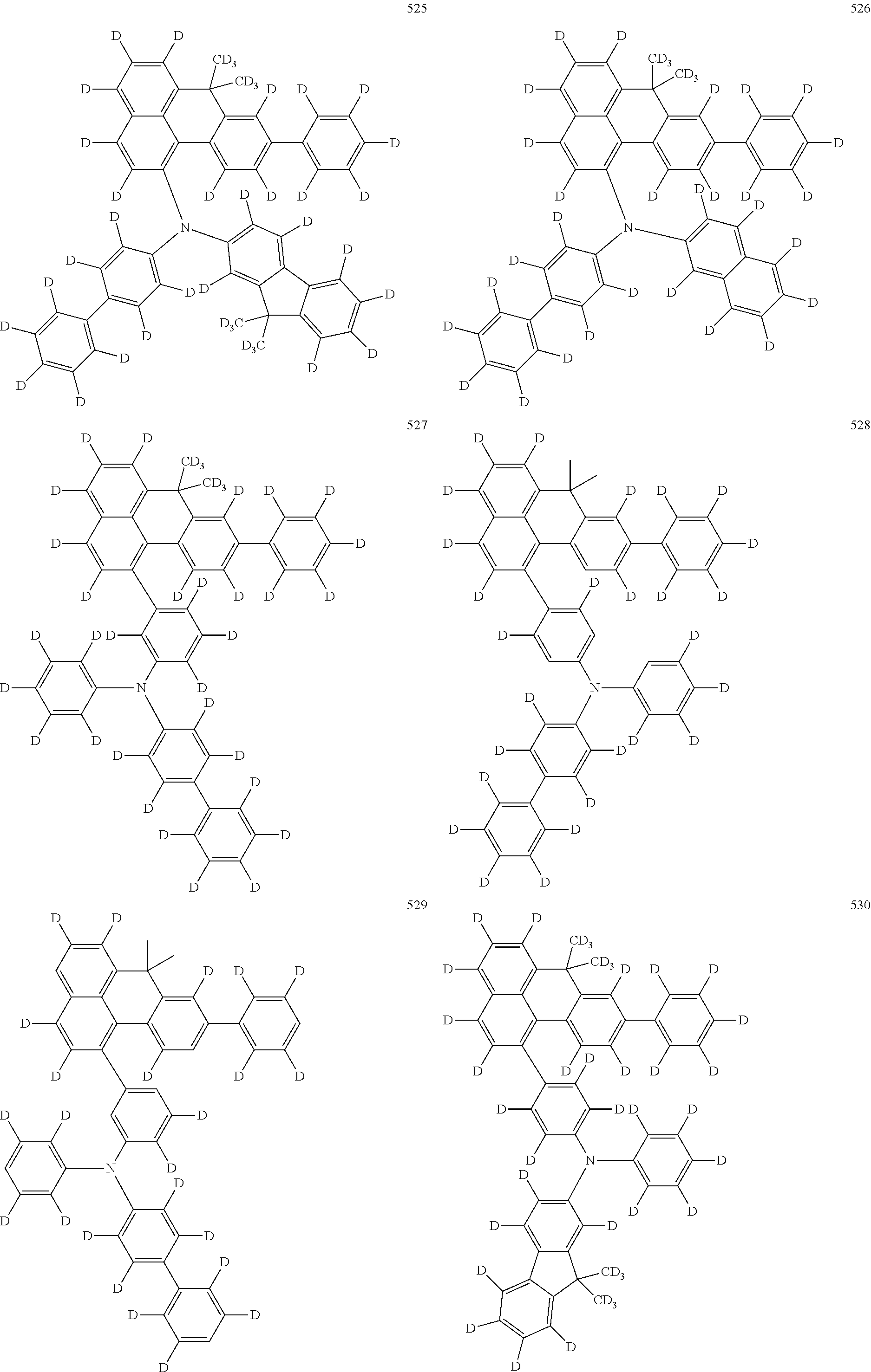

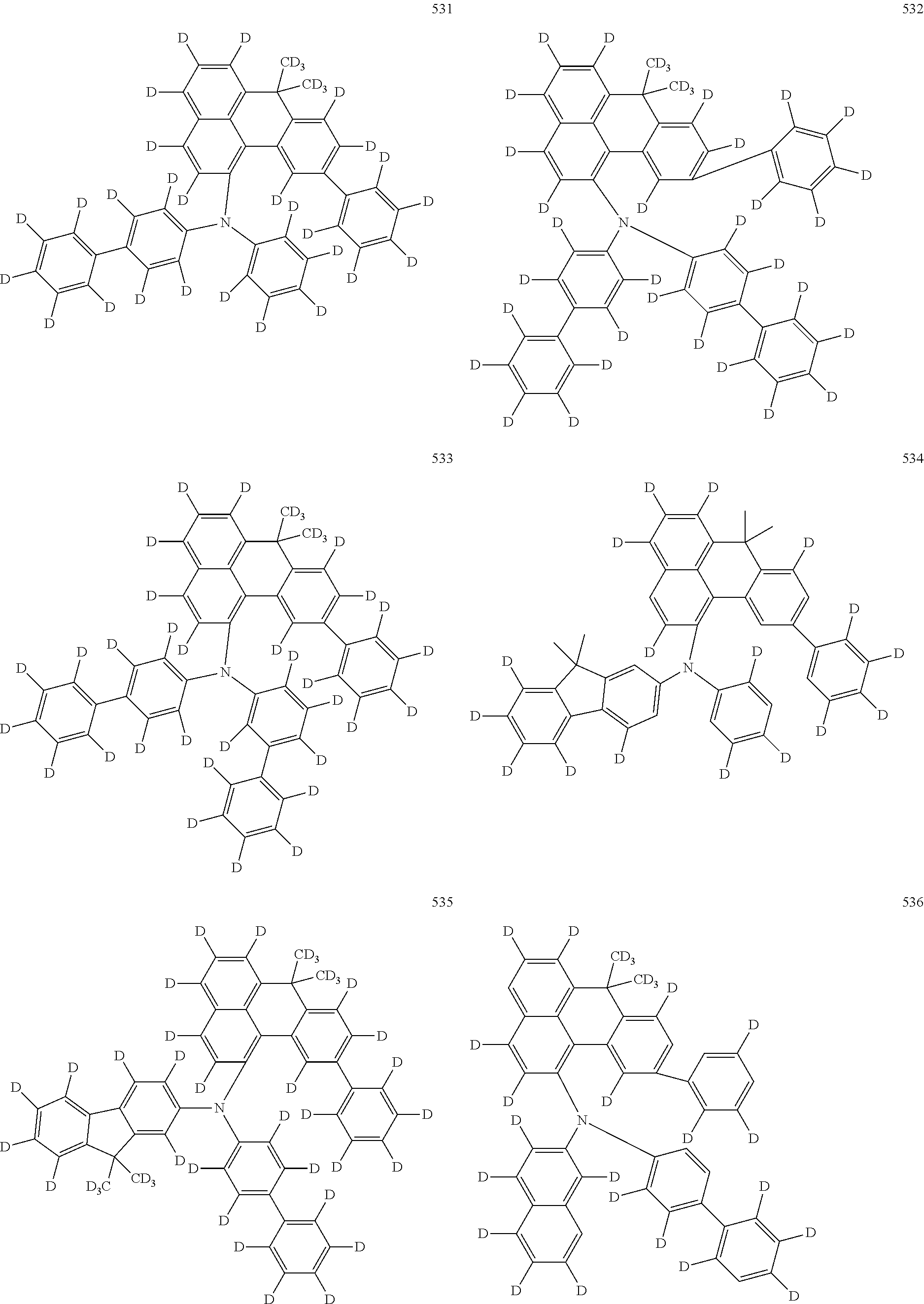

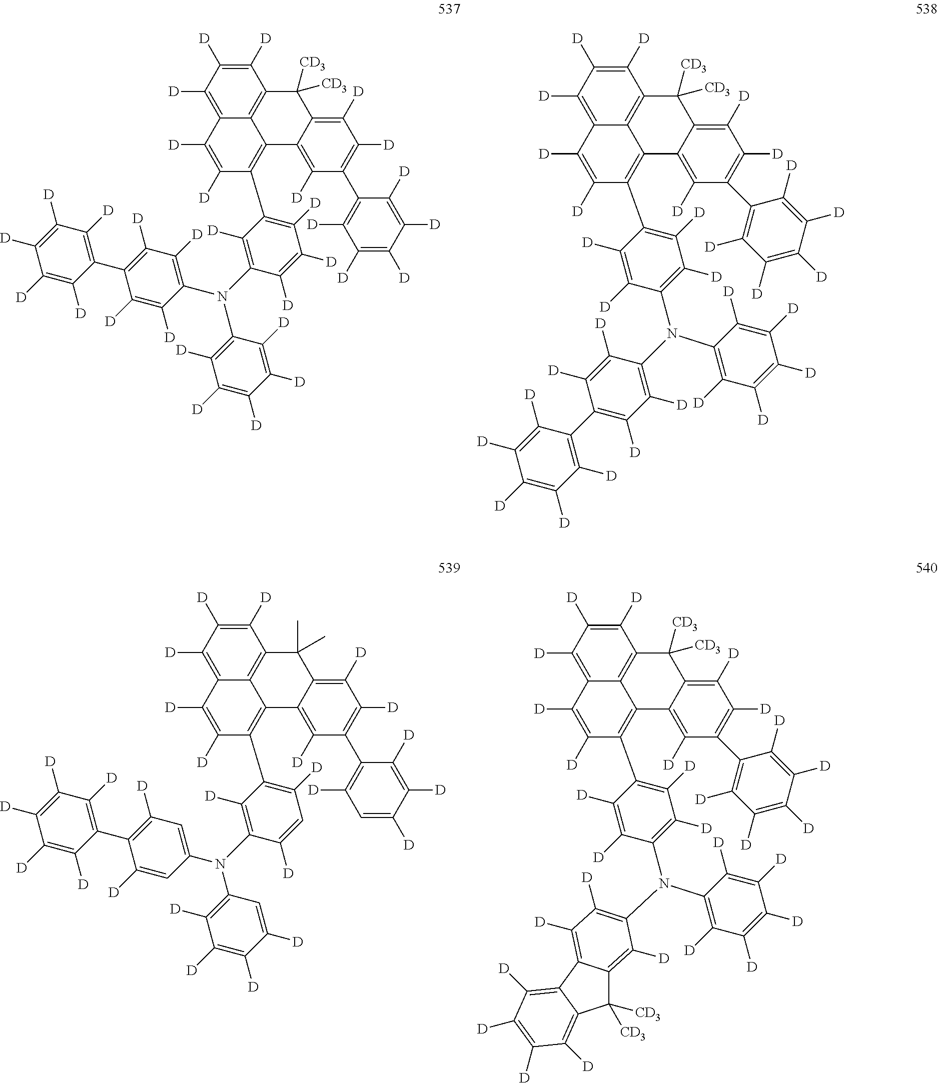

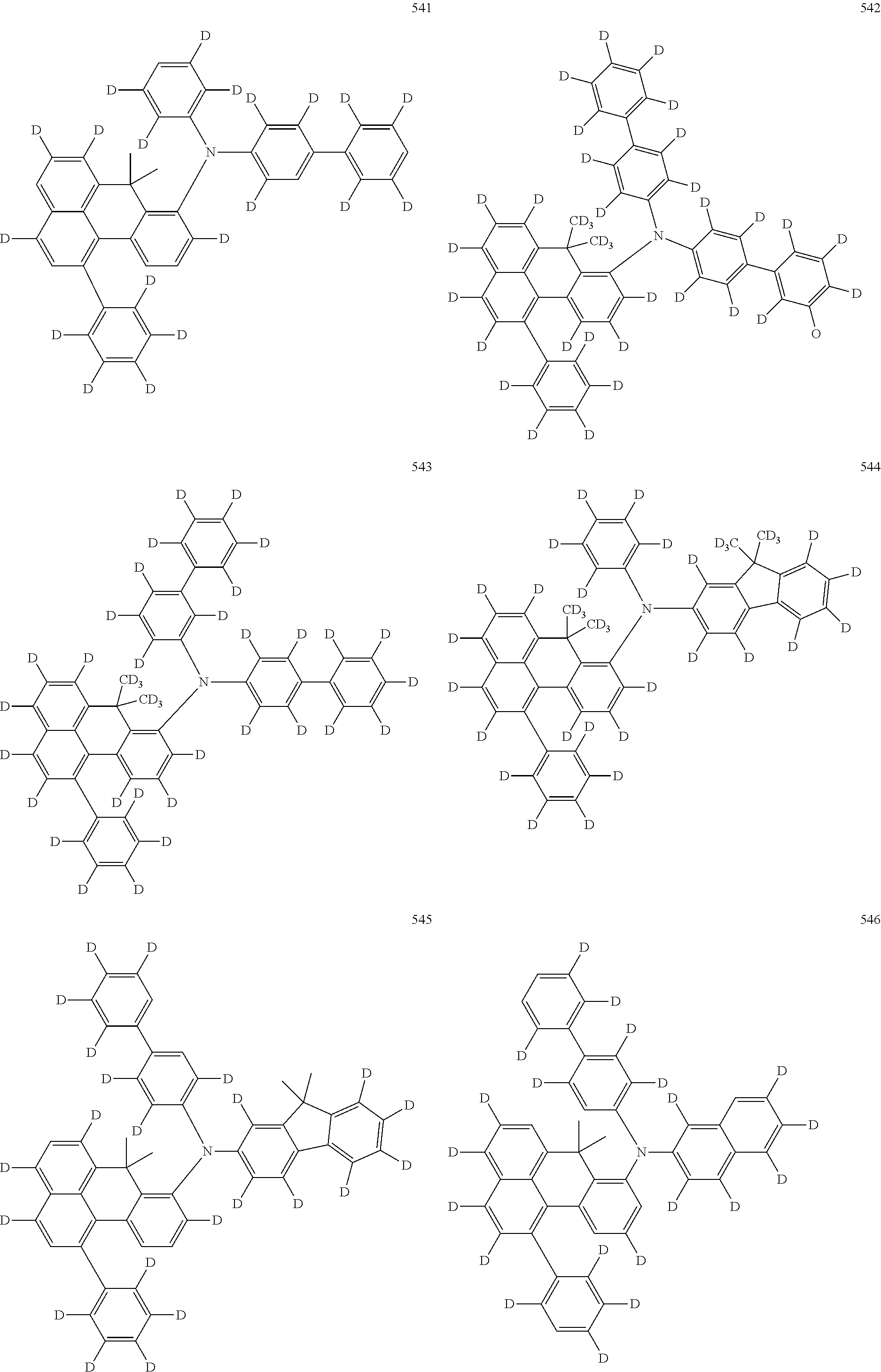

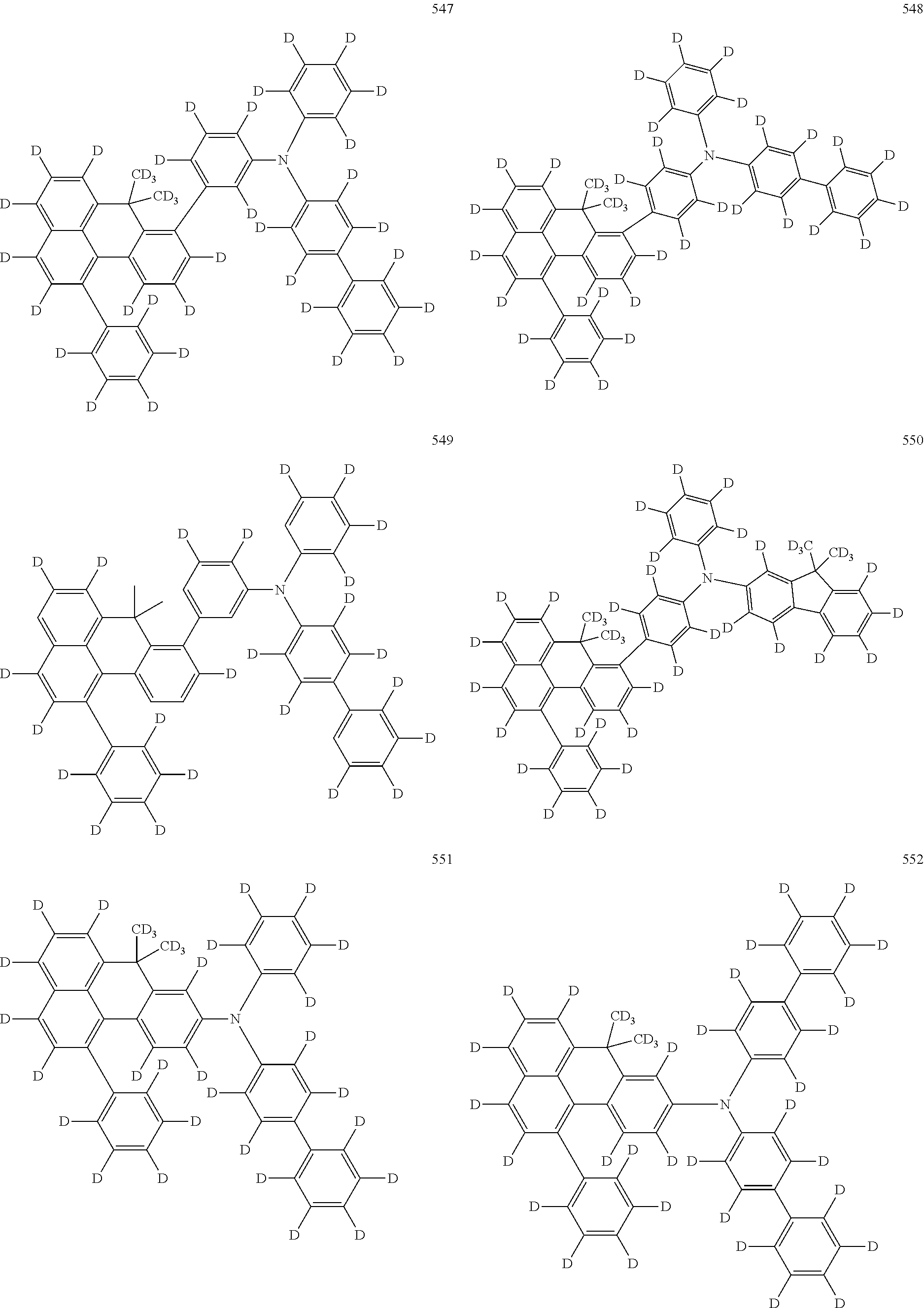

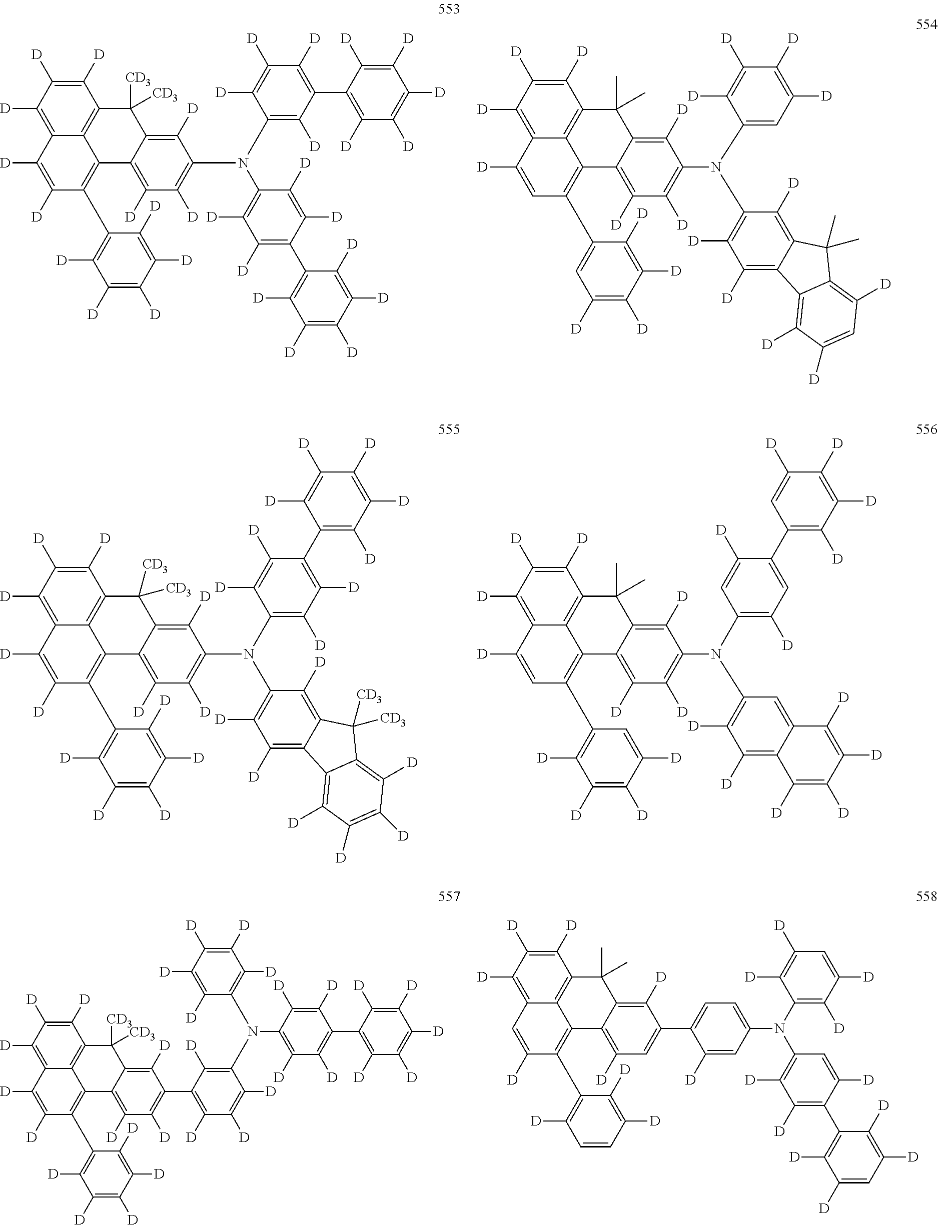

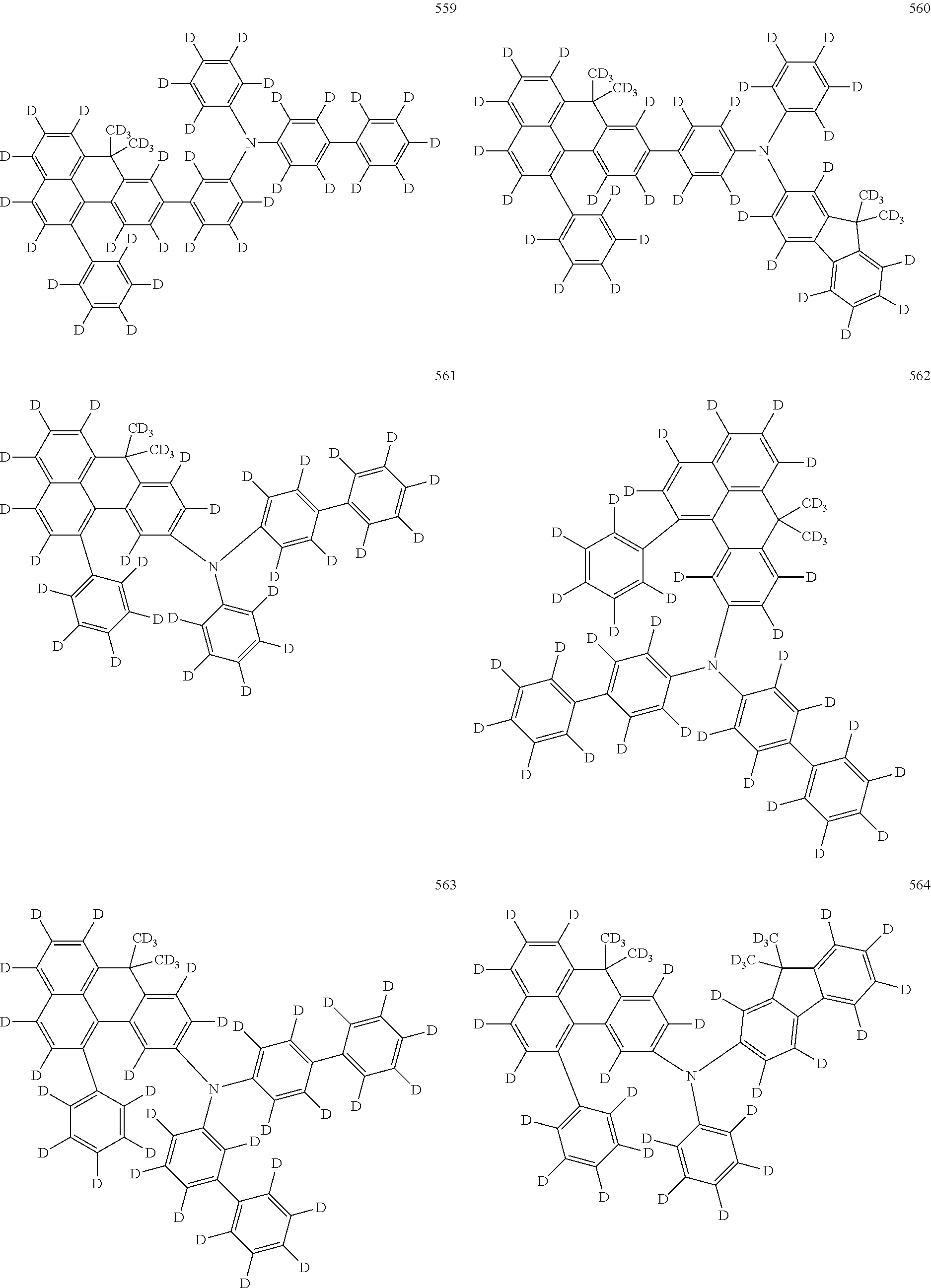

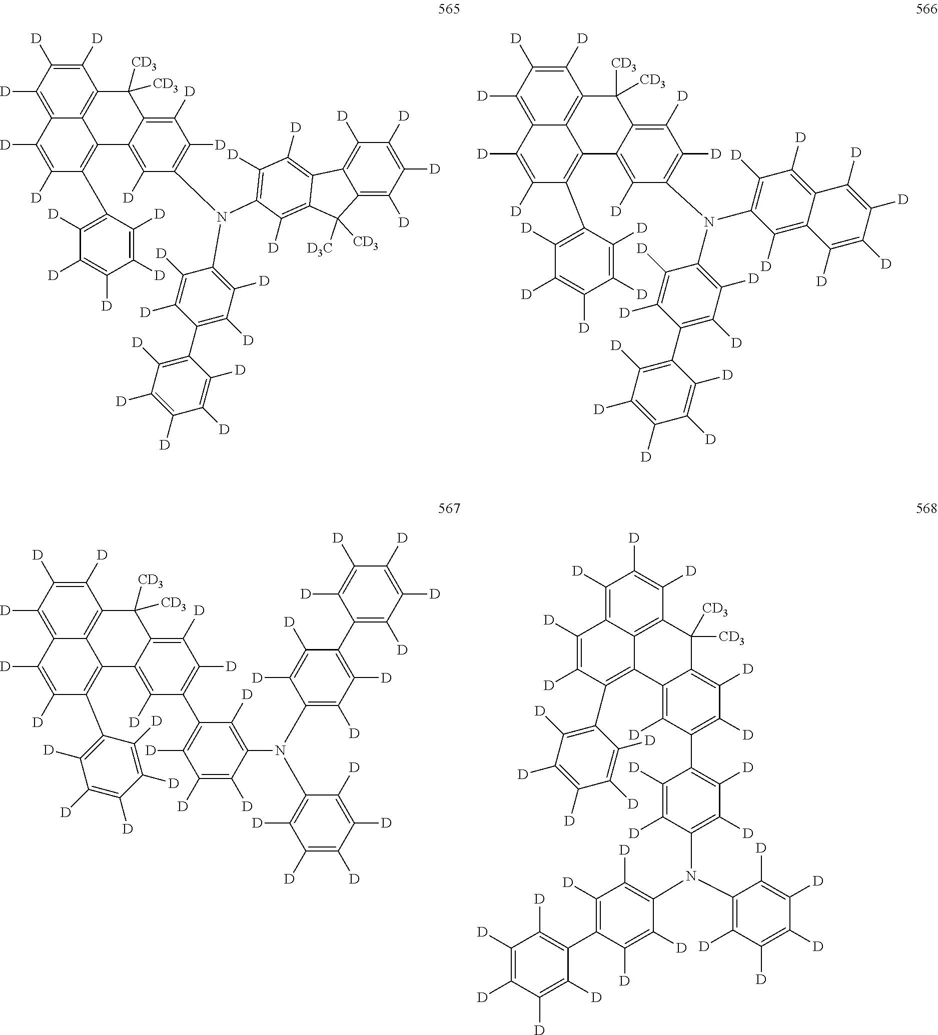

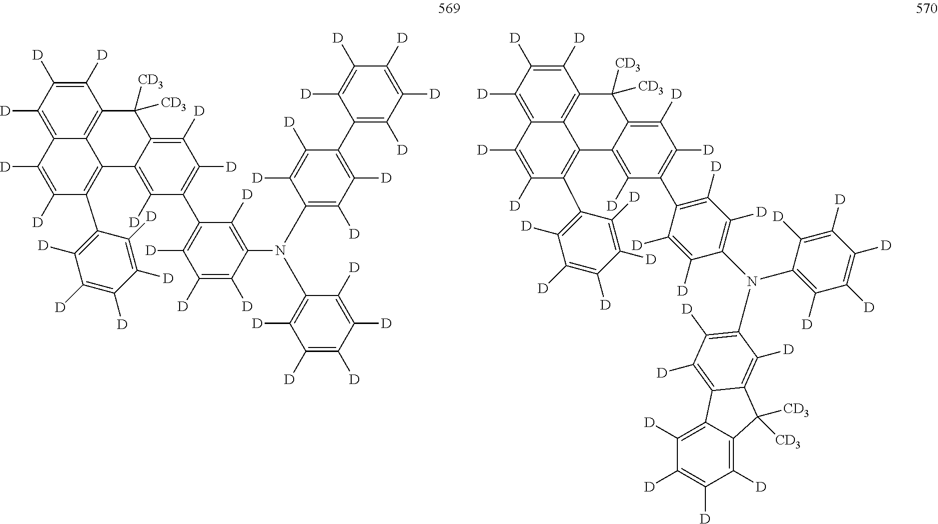

6. An organic light emitting device comprising:
a first electrode;
a second electrode provided to face the first electrode; and
an organic material layer having one or more layers provided between the first electrode and the second electrode,
wherein the one or more layers of the organic material layer comprise one or more of the heterocyclic compound according to claim 1.

7. The organic light emitting device of claim 6, wherein the organic material layer comprises a light emitting layer, and the light emitting layer comprises the heterocyclic compound.

8. The organic light emitting device of claim 6, wherein the organic material layer comprises a light emitting layer, the light emitting layer comprises a host material, and the host material comprises the heterocyclic compound.

9. The organic light emitting device of claim 6, wherein the organic material layer comprises a hole transport layer, and the hole transport layer comprises the heterocyclic compound.

10. The organic light emitting device of claim 6, wherein the organic material layer comprises an electron injection layer or electron transport layer, and the electron injection layer or electron transport layer comprises the heterocyclic compound.

11. The organic light emitting device of claim 6, wherein the organic material layer comprises an electron blocking layer or a hole blocking layer, and the electron blocking layer or the hole blocking layer comprises the heterocyclic compound.

12. The organic light emitting device of claim 6, further comprising one or two or more layers selected from the group consisting of a light emitting layer, a hole injection layer, a hole transport layer, an electron injection layer, an electron transport layer, an electron blocking layer and a hole blocking layer.
